SEQUENCE LISTING (622 sequences)

SEQ ID NO: 1 - protein, 1082 aa, synthetic construct
MAAFKSNPINYILGLDIGIASVGWAMVEIDEEGNPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIAKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPSSKKQRILLQKFDEDGFKECNLNDTRYVNRFLCQFVADHILLTGKGKRRVVASNGQITNLLRGFWRLRKVRAENDRHHALDAVVVACSTVAMQQKITRFVRYKEMNAFDGKTVDKETGKVLYQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVR

SEQ ID NO: 2 - protein, 1082 aa, synthetic construct
MAAFKPNPINYILGLDIGIASVGWAMVEIDEEENPIRLIDLGVRVFKRAEVPKTGDSLAMARRLARSMRRLTRRRAHRLLRARRLLKREGVLQAADFDENGLIKSLPNTPWQLRAAALDRKLAPLEWSAVLLHLIKHRGYLSQRKNEGETAGKKLGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDIAGRLKDRVQPEILEALLKNISFDKFVQISLKSLRRIVPLMEQGKRYDEACAEIYGDRYGKKNTEAKIYLPPIPADEIRNPVVLRALSQTRKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNPREWQEFKARVETSRFPRSKKQRILLQKFDEDGFKECNLNDTRYVSRFLCQFVADHILLTGKGKRRVFASNGQITNLLRGFWGLRKVRAENARHHALDAVVVACSTVAMQQKITRFVRYKEMNAFDGKTIDKETGKALYQKTRFPQPWEFFAQEVMIRVFGKPDGKPEFEEADTPEKLRTLLAEKLSSRPEAAHEYVTPLFVSRAPNRKMSGAHKATLRSAKRFVKHNEKVSVKRVLLTEIKLADLENMVNYKNGREIELYEALKARLEAYGGNAKQAFDPKDNPFYKKGGQLVKAVRVEKTQESGVLLNKKNAYTIADNGDRVRVDVFCKVDKKGKNQYFIVPIYAWQVAENILPDIDCKGYRIDDSYTFCFSLHRYDLIAFQKDEKSKVEFAYYINCNASNGYFYLAWHDKGSKEQQFSISTQNLVLIQKYQVSELGKEIRPCRLKKRPPVR

SEQ ID NO: 3 - protein, 1082 aa, synthetic construct
MAAFKPNPINYILGLDIGIASVGWAMVEIDEEENPIRLIDLGVRVFKRAEVPKTGDSLAMARKLARSMRRLTRRRAHRLLRARRLLKREGVLQAADFDENGLIKSLPNTPWQLRATALDRKLAPLEWSAVLLHLIKHRGYLSQRKNEGETANKKLGALLKGVANNAHALQTGDFRTPAELALNKFEKESGHIRNQRGDYSHTFSRKDLQAELILLFEKQKDFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDIAGRLKDRVQPEILEALLKHISFDKFVQISLKALRRIVPLMEQGKRYDEACAEIYGDRYGKKNTEKKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVSRFLCQFVADHILLTGKGKRRVFASNGQITNLLRGFWGLRKVRAENARHHSLDAVVVACSTVAMQQKITRFVRYKEMNAFDGKTIDKETGKVLHQRTHFPQPWEFFAQEVMIRVFGKPDGKPEFEEADTPEKLRTLLAEKLSIRPEAVHEYVTPLFVSRAPNRKMSGAHKATLRSAKRFVKHNEKISVKRVWLTEIKLADLENMVNYKNGREIELYEALKARLEAYGGNAKQAFDPKDNPFYKKGGQLVKAVRVEKTQKSGVLLNKRNAYTIADNGDRVRVDVFCKVDKKGKNQYFIVPIYAWQVAENILPDIDCKGYRIDDSYTFCFSLHRYDLIAFQKDEKSKVEFAYYINCNASNGNFYLAWHDKGSKEQQFCISTQNLVLIQKYQVNELGKEIRPCRMKKRPPVR

SEQ ID NO: 4 - protein, 1082 aa, synthetic construct
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVR

SEQ ID NO: 5 - protein, 1082 aa, Neisseria meningitidis
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 6 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDAALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 7 - DNA, 7381 bp, synthetic construct
  source 1..7381: mol_type = other DNA
tgagatcctttttttctgcgcgtaatctgctgcttgcaaacaaaaaaaccaccgctaccagcggtggtttgtttgccggatcaagagctaccacctctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacaagaggacatccggtaagaggacatccggtcaaataaaacgaaaggctcagtcgaaagactgggcctttcgttttgctgaggagacttagggactctcacagctaacaccacgtcgtccctatctgctgccctaggtctatgagtggttgctggataactttacgggcatgcataaggctcgtataatatattcagggagaccacaacggtttccctctacaaataattttgtttaacttttactagagtgggaccctacctgcaggtgcagtaaaggaaaaaaaaaatgaaaaaattattattcgcaattcctttatgtctcagctacgaaactgaaatcttgaccgtcgaatatggtctgctgccaatcggcaagattgttgaaaaacgtattgaatgtacggtctactcagtggataacaacggcaatatctacacccagccggtggcccagtggcatgaccgtggtgaacaggaagtgttcgaatattgtctggaagacggatctttaatccgtgccacaaaggatcacaaatttatgactgtagatggtcagatgctcccaatcgacgaaatttttgaacgcgaattagacctgatgcgcgtggataatctcccgaattccggaggatctagcggggnrncctctggctctgagtagtgaggaacaagcgagagtgcaacacctgaaagcagcgggggcagcagcggggggtcatctggaggaagttcgggaggaagttctggcagcgagacaccaggaacaagcgagtcagcaacaccagagagcagtggcggctcgagcggtggtagctctggtgggtcaaaaagaaccgccgacggcagcgaattcgagcccaagaagaagaggaaagtcggatcctacccatacgatgttccagattacgcttatccctacgacgtgcctgattatgcatacccatatgatgtccccgactatgccggggggtcaagcggggggtcaatcaaaattgccacgcgtaaatatttaggcaaacagaatgtttatgatatcggtgtcgagcgcgatcataatttcgcgctgaaaaacggatttatagctagcaattgtttcaacgttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaatgaaatttggaaactttttgcttacataccaacctccccaattttcccaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattatggcggtggcggtagcggcggtggcggtagcggcggtggcggtagcggcggtggcggtagcaaatttggattgttcttccttaacttcatcaattcaacaactgttcaagaacagagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtccgcatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacacggaatatacctaaacggagccaatgtacgcaaaaaaccccgcttcagcggggttttttcgccaaaaaaaaccccgccctgtcaggggcggggtttttttttagggccctactgactgtaaaaaaataaacgatgccccttaaagcagaagctttaaggggcagagcgttgcggcacatcttttcagacggccttattgtagcaacggttctcatttcgtttccgaaatgagaaagggagctacaacctggctctgagtagtgaggaacaccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcacttaattaacggcactgcgaaaaaaccccgccgaagcggggttttttgcgaaatataatgaccctcttgataacccaagagggcattttttaatgcccatggcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttaggccaaataggccgt

SEQ ID NO: 8 - DNA, 7114 bp, synthetic construct
  source 1..7114: mol_type = other DNA
tgagatcctttttttctgcgcgtaatctgctgcttgcaaacaaaaaaaccaccgctaccagcggtggtttgtttgccggatcaagagctaccacctctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacaagaggacatccggtaagaggacatccggtcaaataaaacgaaaggctcagtcgaaagactgggcctttcgttttgctgaggagacttagggactctcacagctaacaccacgtcgtccctatctgctgccctaggtctatgagtggttgctggataactttacgggcatgcataaggctcgtataatatattcagggagaccacaacggtttccctctacaaataattttgtttaacttttactagagtgggaccctacctgcaggtgcagtaaaggaaaaaaaaaatgaaaaaattattatttgcgataccgctgtgcttaagctacgaaactgaaatcttgaccgtcgaatatggtctgctgccaatcggcaagattgttgaaaaacgtattgaatgtacggtctactcagtggataacaacggcaatatctacacccagccggtggcccagtggcatgaccgtggtgaacaggaagtgttcgaatattgtctggaagacggatctttaatccgtgccacaaaggatcacaaatttatgactgtagatggtcagatgctcccaatcgacgaaatttttgaacgcgaattagacctgatgcgcgtggataatctcccgaattccggaggatctagcggggcaacctctggctctgagtagtgaggaacaagcgagagtgcaacacctgaaagcagcgggggcagcagcggggggtcaatcaaaattgccacgcgtaaatatttaggcaaacagaatgtttatgatatcggtgtcgagcgcgatcataatttcgcgctgaaaaacggatttatagctagcaattgtttcaacgttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaatgaaatttggaaactttttgcttacataccaacctccccaattttcccaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattatggcggtggcggtagcggcggtggcggtagcggcggtggcggtagcggcggtggcggtagcaaatttggattgttcttccttaacttcatcaattcaacaactgttcaagaacagagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtccgcatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacacggaatatacctaaacggagccaatgtacgcaaaaaaccccgcttcagcggggttttttcgccaaaaaaaaccccgccctgtcaggggcggggtttttttttagggccctactgactgtaaaaaaataaacgatgccccttaaagcagaagctttaaggggcagagcgttgcggcacatcttttcagacggccttattgtagcaacggttctcatttcgtttccgaaatgagaaagggagctacaacctggctctgagtagtgaggaacaccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcacttaattaacggcactgcgaaaaaaccccgccgaagcggggttttttgcgaaatataatgaccctcttgataacccaagagggcattttttaatgcccatggcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttaggccaaataggccgt

SEQ ID NO: 9 - DNA, 3821 bp, synthetic construct
  source 1..3821: mol_type = other DNA
tcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaagaggacatccggtcaaataaaacgaaaggctcagtcgaaagactgggcctttcgttttgctgaggagacttagggaccctgatgaaattcgccacttgttagtgtaattcgctaactcatcctggcatgttgctgttgattcttcaatcagatctttataaatcaaaaagataaaaaattggcacgcaaattgtattaacagttcagcaggacaatcctgaacgcagaaatcaagaggacaacattacctgcaggtgcagtaaaggaaaaaaaaaatgtctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgagaaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaacaaggaatctggctctgtatcttctgaacagttggcgcaattccgctctctggacagccagctgaaccgctgcctgagcctggataccatggtggtgaccaacggcaaagcgattgaaattcgcgatgtgaaagtgggcgattggctggaaagcgaatgcggcccggtgcaggtgaccgaagtgctgccgattattaaacagccggtgtttgaaattgtgctgaaaagcggcaaaaaaattcgcgtgagcgcgaaccataaatttccgaccaaagatggcctgaaaaccattaacagcggcctgaaagtgggcgattttctgcgcagccgcgcgaaataacttaattaacggcactcctcagcaaatataatgaccctcttgataacccaagagggcattttttaatgcccatggcgtttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgaaggccaaataggccgt

SEQ ID NO: 10 - DNA, 7187 bp, synthetic construct
  source 1..7187: mol_type = other DNA
tgagatcctttttttctgcgcgtaatctgctgcttgcaaacaaaaaaaccaccgctaccagcggtggtttgtttgccggatcaagagctaccacctctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacaagaggacatccggtaagaggacatccggtcaaataaaacgaaaggctcagtcgaaagactgggcctttcgttttatggaaagaagagaaaccgggactggttcgttttgctgaggagacttagggactctcacagctaacaccacgtcgtccctatctgctgccctaggtctatgagtggttgctggataactttacgggcatgcataaggctcgtataatatattcagggagaccacaacggtttccctctacaaataattttgtttaacttttactagagtgggaccctacctgcaggtgcagtaaaggaaaaaaaaaatgaaaaaattattatttgcgataccgctgtgcttaagctacgaaactgaaatcttgaccgtcgaatatggtctgctgccaatcggcaagattgttgaaaaacgtattgaatgtacggtctactcagtggataacaacggcaatatctacacccagccggtggcccagtggcatgaccgtggtgaacaggaagtgttcgaatattgtctggaagacggatctttaatccgtgccacaaaggatcacaaatttatgactgtagatggtcagatgctcccaatcgacgaaatttttgaacgcgaattagacctgatgcgcgtggataatctcccgaattccggaggatctagcccaacctctggctctgagtagggcggaacaggatctagcgggggttcaagcgagagtgcaacacctgaaagcagcgggacaaaagctggctctgagtagggcggaacaagcggtggctcaatcaaaattgccacgcgtaaatatttaggcaaacagaatgtttatgatatcggtgtcgagcgcgatcataatttcgcgctgaaaaacggatttatagctagcaattgtttcaacgttgttcctttctattctcactccgccgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaatgaaatttggaaactttttgcttacataccaacctccccaattttcccaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattatggcggtggcggtagcggcggtggcggtagcggcggtggcggtagcggcggtggcggtagcaaatttggattgttcttccttaacttcatcaattcaacaactgttcaagaacagagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtccgcatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacacggaatatacctaaacggagccaatgtacgcaaaaaaccccgcttcagcggggttttttcgccaaaaaaaaccccgccctgtcaggggcggggtttttttttagggccctactgactgtaaaaaaataaacgatgccccttaaagcagaagctttaaggggcagagcgttgcggcacatcttttcagacggccttattgtagcaacggttctcatttcgtttccgaaatgagaaagggagctacaacctggctctgagtagggcggaacaccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcacttaattaacggcactgcgaaaaaaccccgccgaagcggggttttttgcgaaatataatgaccctcttgataacccaagagggcattttttaatgcccatggcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttaggccaaataggccgt

SEQ ID NO: 11 - protein, 1082 aa, synthetic construct
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRCDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNERGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRILLAEKLSSRPEAVHEYVTPLFVSRAPNRKMSGAHKGTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYSNCDSSNGKFYLAWHDKGRKEQQFRISTQNLVLIQKYQVNELGKEIRPCRMKKRPPVR

SEQ ID NO: 12 - protein, 1082 aa, synthetic construct
MAAFKPNPINYILGLDIGIASVGWAMVEIDEEENPIRLIDLGVRVFKRAEVPKTGDSLAMARRLARSMRRLTRRRAHRLLRARRLLKREGVLQAADFDENGLIKSLPNTPWQLRAAALDRKLAPLEWSAVLLHLIKHRGYLSQRKNEGETADKKLGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALAKEGLKDKKSPLNLSSELQDEIGTAFSLFKTDEDIAGRLKDRVQPEILEALLKHISFDKFVQISLKALRRIVPLMEQGKRYDEACAEIYGDRYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENARHHALDAVVVACSTVAMQQKITRFVRYKEMNAFDGKTIDKETGKVLYRKTLFPQPWEFFAQEVMIRVFGKPDGKPEFEEADTPEKLRTLLAERLSSRPEAVHEYVTPLFVSRAPNRKMSGAHKATLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDRVRVDVFCKVDKKGKNQYFIVPIYAWQVAENILPDIDCKGYRIDDSYTFCFSLHKYDLIAFQKDEKSKVEFAYYINCDSSNGKFYLAWHDKGSKEQQFRISTQNLVLIQKYQVNELGKEIRPCRLKKRPPVR

SEQ ID NO: 13 - protein, 1082 aa, synthetic construct
MAAFKPNPINYILGLDIGIASVGWAMVEIDEEENPIRLIDLGVRVFKRAEVPKTGDSLAMARRLARSMRRLTRRRAHRLLRARRLLKREGVLQAADFDENGLIKSLPNTPWQLRAAALDRKLAPLEWSAVLLHLIKHRGYLSQRKNEGETADKKLGALLKGVANNAHALQTGDFRTPAELALNKFEKESGHIRNQRGDYSHTFSRKDLQSELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDIAGRLKNRVQPEILEALLKHISFDKFVQISLKALRRIVPLMEQGKRYDEACAEIYGDCYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDYSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENARHHALDAVVVACSTVAMQQKITRFVRYKETNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKATLRSAKRFVKHNEKISVKRVWLTEIKLADLENMVNYKNGREIELYEALKVRLEAYGGNAKQAFDPKDNPFYKKGGQLVKAVRVEKTQESGVLLNKKNAYTIADNGDRVRVDVFCKVDKKGKNQYFIVPIYAWQVAENILPDIDCKGYRIDDSYTFCFSLHKYDLIAFQKDEKSKVEFAYYINCDSSNGKFYLAWHDKGSKEQQFRISTQNLVLIQKYQVNELGKEIRPCRLKKRPPVR

SEQ ID NO: 14 - protein, 1082 aa, synthetic construct
MAAFKPNPINYILGLDIGIASVGWAMVEIDEEENPIRLIDLGVRVFQRAEVPKTGDSLAMARRLARSVRRLTRRRAHRLLRARRLLKREGVLQAADFDENGLIKSLPHTPWQLRTAALDRKLTPLEWSAVLLHLIKHRGYLSQRKNEGETADKKLGALLKGVANNAHALQTGDFRTPAELALNKFEKESGHIRNQRGDYSHTFSRKNLQSGLILLFEKQKEFGNPHVSGGLKEGIETLLMTQRPALSGDAVQKMLGHCTFESAEPKAAKNTYTAERFIWLTKLNNLRILEQGSERPLTDTERATLMDEPYRKSKLTYAQARKLLGLEDTAFFKGLRYVKDNAEVSTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVRYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEGGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKEAGKALYQKARFPQPWEFFAQEVMIRVFGKPDGKPEFEEADTPEKLRTLLAERLSSRPEAVHEYVTPLFVSRAPNRKMSGAHKVILRSAKRFVKHNEKISVKRVWLTEIKLADLENMVNYRNGREIELYEALKARLEAYGGNAKQAFDPKDNPFYKKGGQLVKAVRVERTQERGVLLNKKNAYTIADNGDMVRVDVFCKVGKKGKNQYFIVPIYAWQVAENILPDIDCKGYRIDDSYTFCFSLHKYDLIAFQKDEKSKVEFAYYSYCHSSSGDFYLAWHDKGSKEQQFRISTQNLVLIQKYQVNELGKEIRPCRLKKRPLVR

SEQ ID NO: 15 - protein, 5 aa, synthetic construct
DRYSK

SEQ ID NO: 16 - protein, 5 aa, Neisseria meningitidis
DHYGK

SEQ ID NO: 17 - protein, 21 aa, synthetic construct
GGSGGSGGSGGSGGSGGSGGS

SEQ ID NO: 18 - DNA, 327 bp, synthetic construct
  source 1..327: mol_type = other DNA
aaacaaagcactattgcactgtgtctcagctacgaaaccgaaatcttgaccgtcgaatatggtctgctgccaatcggcaagattgttgaaaaacgtattgaatgtacggtctactcagtggataacaacggcaatatctacacccagccggtggcccagtggcatgaccgtggtgaacaggaagtgttcgaatattgtctggaagacggatctttaatccgtgccacaaaggatcacaaatttatgactgtagatggtcagatgctcccaatcgacgaaatttttgaacgcgaattagacctgatgcgcgtggataatctcccgaat

SEQ ID NO: 19 - DNA, 156 bp, synthetic construct
  source 1..156: mol_type = other DNA
atgatcaaaattgccacgcgtaaatatttaggcaaacagaatgtttatgatatcggtgtcgagcgcgatcataatttcgcgctgaaaaacggctttatcgccagcaattgttttaatgcactcttaccgttactgtttacccctgtgactaaagcc

SEQ ID NO: 20 - RNA, 166 nt, synthetic construct
  source 1..166: mol_type = other RNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttcaagaaagtgaatgaaggaatgcaac

SEQ ID NO: 21 - protein, 160 aa, synthetic construct
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 22 - protein, 161 aa, synthetic construct
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 23 - protein, 183 aa, synthetic construct
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 24 - protein, 164 aa, synthetic construct
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 25 - protein, 173 aa, synthetic construct
  source 1..173: note = strain F3031
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 26 - protein, 161 aa, synthetic construct
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 27 - protein, 19 aa, synthetic construct
NLSKRPAAIKKAGQAKKKK

SEQ ID NO: 28 - protein, 84 aa, unidentified
  source 1..84: note = Bacillus phage PBS2
MTNLSDIIEKETGKQLVIQESILMLPEEVEEVIGNKPESDILVHTAYDESTDENVMLLTSDAPEYKPWALVIQDSNGENKIKML

SEQ ID NO: 29 - protein, 11 aa, synthetic construct
RQRRNELKRSF

SEQ ID NO: 30 - DNA, 21 bp, synthetic construct
  source 1..21: mol_type = other DNA
caccgttcatctgtcctcttt

SEQ ID NO: 31 - DNA, 22 bp, synthetic construct
  source 1..22: mol_type = other DNA
cgacctgctccatgttacttag

SEQ ID NO: 32 - DNA, 23 bp, synthetic construct
  source 1..23: mol_type = other DNA
gcagatcccacaggcgccctggc

SEQ ID NO: 33 - protein, 198 aa, synthetic construct [sequence omitted]

SEQ ID NO: 34 - protein, 198 aa, synthetic construct
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 35 - protein, 198 aa, synthetic construct
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 36 - protein, 199 aa, synthetic construct
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 37 - protein, 429 aa, synthetic construct
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 38 - protein, 429 aa, synthetic construct
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 39 - protein, 370 aa, synthetic construct
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 40 - protein, 384 aa, synthetic construct
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 41 - protein, 377 aa, synthetic construct
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 42 - protein, 384 aa, synthetic construct
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 43 - protein, 373 aa, synthetic construct
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 44 - protein, 382 aa, synthetic construct
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 45 - protein, 190 aa, synthetic construct
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 46 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 47 - protein, 200 aa, synthetic construct
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 48 - protein, 386 aa, synthetic construct
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 49 - protein, 236 aa, synthetic construct
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 50 - protein, 229 aa, synthetic construct
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 51 - protein, 229 aa, synthetic construct [sequence omitted]

SEQ ID NO: 52 - protein, 208 aa, synthetic construct
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 53 - protein, 208 aa, synthetic construct
MTDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 54 - protein, 384 aa, synthetic construct
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 55 - protein, 184 aa, synthetic construct
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 56 - protein, 184 aa, synthetic construct
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 57 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 58 - DNA, 20 bp, synthetic construct
  source 1..20: mol_type = other DNA
gatcccacaggcgccctggc

SEQ ID NO: 59 - DNA, 23 bp, synthetic construct
  source 1..23: mol_type = other DNA
gcatttcccacaggcgccctggc

SEQ ID NO: 60 - DNA, 20 bp, synthetic construct
  source 1..20: mol_type = other DNA
tttcccacaggcgccctggc

SEQ ID NO: 61-62 - skipped (2 entries)

SEQ ID NO: 63 - DNA, 23 bp, synthetic construct
  source 1..23: mol_type = other DNA
tgaggtcccacaggccccctggc

SEQ ID NO: 64 - DNA, 20 bp, synthetic construct
  source 1..20: mol_type = other DNA
ggtcccacaggccccctggc

SEQ ID NO: 65 - DNA, 23 bp, synthetic construct
  source 1..23: mol_type = other DNA
tgggctcccacaggccccctggc

SEQ ID NO: 66 - DNA, 20 bp, synthetic construct
  source 1..20: mol_type = other DNA
gctcccacaggccccctggc

SEQ ID NO: 67 - DNA, 23 bp, synthetic construct
  source 1..23: mol_type = other DNA
agagataccacaggcaccctggc

SEQ ID NO: 68 - DNA, 20 bp, synthetic construct
  source 1..20: mol_type = other DNA
gataccacaggcaccctggc

SEQ ID NO: 69 - DNA, 23 bp, synthetic construct
  source 1..23: mol_type = other DNA
gctgctcccagaggcgccctggc

SEQ ID NO: 70 - DNA, 20 bp, synthetic construct
  source 1..20: mol_type = other DNA
gctcccagaggcgccctggc

SEQ ID NO: 71 - DNA, 23 bp, synthetic construct
  source 1..23: mol_type = other DNA
agagatcacccaggcgccctggc

SEQ ID NO: 72 - DNA, 20 bp, synthetic construct
  source 1..20: mol_type = other DNA
gatcacccaggcgccctggc

SEQ ID NO: 73 - DNA, 23 bp, synthetic construct
  source 1..23: mol_type = other DNA
tgggaccccacatgcgccctggc

SEQ ID NO: 74 - DNA, 20 bp, synthetic construct
  source 1..20: mol_type = other DNA
gaccccacatgcgccctggc

SEQ ID NO: 75 - DNA, 23 bp, synthetic construct
  source 1..23: mol_type = other DNA
gctgcagaagggattccatgagg

SEQ ID NO: 76 - DNA, 20 bp, synthetic construct
  source 1..20: mol_type = other DNA
gcagaagggattccatgagg

SEQ ID NO: 77 - DNA, 23 bp, synthetic construct
  source 1..23: mol_type = other DNA
tgtgaagaagggtttccatgagg

SEQ ID NO: 78 - RNA, 91 nt, synthetic construct
  source 1..91: mol_type = other RNA
gttttagtactctgtaatgaaaattacagaatctactaaaacaaggcaaaatgccgtgtttatctcgtcaacttgttggcgagattttttt

SEQ ID NO: 79 - DNA, 20 bp, synthetic construct
  source 1..20: mol_type = other DNA
gaagaagggtttccatgagg

SEQ ID NO: 80 - DNA, 23 bp, synthetic construct
  source 1..23: mol_type = other DNA
gattctgaagggattccatgagg

SEQ ID NO: 81 - DNA, 20 bp, synthetic construct
  source 1..20: mol_type = other DNA
tctgaagggattccatgagg

SEQ ID NO: 82 - DNA, 23 bp, synthetic construct
  source 1..23: mol_type = other DNA; sub_species = familiaris
catggagaagggatcccatgagg

SEQ ID NO: 83 - DNA, 20 bp, synthetic construct
  source 1..20: mol_type = other DNA
ggagaagggatcccatgagg

SEQ ID NO: 84 - DNA, 23 bp, synthetic construct
  source 1..23: mol_type = other DNA
ggtacagaagggcttccatgagg

SEQ ID NO: 85 - DNA, 20 bp, synthetic construct
  source 1..20: mol_type = other DNA
acagaagggcttccatgagg

SEQ ID NO: 86 - DNA, 23 bp, synthetic construct
  source 1..23: mol_type = other DNA
ggagcagaaggcatttcatgagg

SEQ ID NO: 87 - DNA, 20 bp, synthetic construct
  source 1..20: mol_type = other DNA
gcagaaggcatttcatgagg

SEQ ID NO: 88 - DNA, 23 bp, synthetic construct
  source 1..23: mol_type = other DNA
caggcagaaaggaatccatgagg

SEQ ID NO: 89 - DNA, 20 bp, synthetic construct
  source 1..20: mol_type = other DNA
gcagaaaggaatccatgagg

SEQ ID NO: 90 - DNA, 23 bp, synthetic construct
  source 1..23: mol_type = other DNA
aaggcagatgggattccttgagg

SEQ ID NO: 91 - DNA, 20 bp, synthetic construct
  source 1..20: mol_type = other DNA
gcagatgggattccttgagg

SEQ ID NO: 92 - DNA, 23 bp, synthetic construct
  source 1..23: mol_type = other DNA
aaggcagaaggaattccatgagt

SEQ ID NO: 93 - DNA, 20 bp, synthetic construct
  source 1..20: mol_type = other DNA
gcagaaggaattccatgagt

SEQ ID NO: 94 - DNA, 23 bp, synthetic construct
  source 1..23: mol_type = other DNA
cttgctgaagggattcaatgagg

SEQ ID NO: 95 - DNA, 20 bp, synthetic construct
  source 1..20: mol_type = other DNA
gctgaagggattcaatgagg

SEQ ID NO: 96 - DNA, 23 bp, synthetic construct
  source 1..23: mol_type = other DNA
gaggcaggagggattccctgagg

SEQ ID NO: 97 - DNA, 20 bp, synthetic construct
  source 1..20: mol_type = other DNA
gcaggagggattccctgagg

SEQ ID NO: 98 - DNA, 23 bp, synthetic construct
  source 1..23: mol_type = other DNA
agagcggaagggatgccatgagg

SEQ ID NO: 99 - DNA, 20 bp, synthetic construct
  source 1..20: mol_type = other DNA
gcggaagggatgccatgagg

SEQ ID NO: 100 - DNA, 121 bp, synthetic construct
  source 1..121: mol_type = other DNA
gttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 101 - DNA, 23 bp, synthetic construct
  source 1..23: mol_type = other DNA
gttcctcactactcagagccaga

SEQ ID NO: 102 - DNA, 23 bp, synthetic construct
  source 1..23: mol_type = other DNA
tgttcctcactactcagagccag

SEQ ID NO: 103 - DNA, 23 bp, synthetic construct
  source 1..23: mol_type = other DNA
ttcctcactactcagagccagag

SEQ ID NO: 104 - DNA, 23 bp, synthetic construct
  source 1..23: mol_type = other DNA
tgttccgccctactcagagccag

SEQ ID NO: 105 - DNA, 20 bp, synthetic construct
  source 1..20: mol_type = other DNA
ggcccagactgagcacgtga

SEQ ID NO: 106 - DNA, 22 bp, synthetic construct
  source 1..22: mol_type = other DNA
atggcccagactgagcacgtga

SEQ ID NO: 107 - DNA, 23 bp, synthetic construct
  source 1..23: mol_type = other DNA
gattgcgtacttcgaaaaggtag

SEQ ID NO: 108 - DNA, 22 bp, synthetic construct
  source 1..22: mol_type = other DNA
gttagcagacccagatagacag

SEQ ID NO: 109 - DNA, 22 bp, synthetic construct
  source 1..22: mol_type = other DNA
gccctgcccacacccccactcc

SEQ ID NO: 110 - DNA, 23 bp, synthetic construct
  source 1..23: mol_type = other DNA
gaccctagcctaatgccccctgc

SEQ ID NO: 111 - protein, 179 aa, synthetic construct
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 112 - protein, 171 aa, synthetic construct
MPYSLEEQTYFMQEALKEAEKSLQKAEIPIGCVIVKDGEIIGRGHNAREESNQAIMHAEIMAINEANAHEGNWRLLDTTLFVTIEPCVMCSGAIGLARIPHVIYGASNQKFGGADSLYQILTDERLNHRVQVERGLLAADCANIMQTFFRQGRERKKIAKHLIKEQSDPFD

SEQ ID NO: 113 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 114 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNAKTGAAGSLMDVLHYPGMNHRVEITEGILADECAALLCYFFRMPRQVFNAQKKAQSSTD

SEQ ID NO: 115 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 116 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 117 - protein, 151 aa, synthetic construct
MGKEYFLKVALREAKRAFEKGEVPVGAIIVKEGEIISKAHNSVEELKDPTAHAEMLAIKEACRRLNTKYLEGCELYVTLEPCIMCSYALVLSRIEKVIFSALDKKHGGVVSVFNILDEPTLNHRVKWEYYPLEEASELLSEFFKKLRNNII

SEQ ID NO: 118 - protein, 166 aa, synthetic construct
SEVEFSHEYWMRHALTLAKRARDEGEVPVGAVLVLNNRVIGEGWNRAIGLYDPTAHAEIMALRQGGLVMQNYRLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 119 - DNA, 23 bp, synthetic construct
  source 1..23: mol_type = other DNA
aaaggagaaggaattccatgagg

SEQ ID NO: 120 - DNA, 20 bp, synthetic construct
  source 1..20: mol_type = other DNA
ggagaaggaattccatgagg

SEQ ID NO: 121 - DNA, 23 bp, synthetic construct
  source 1..23: mol_type = other DNA
agacttctccacaggagtcaggt

SEQ ID NO: 122 - protein, 166 aa, synthetic construct
SEVEFSHEYWMRHALTLAKRARDEGEVPVGAVLVLNNRVIGEGWNRAIGLYDPTAHAEIMALRQGGLVMQNYGLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 123 - protein, 166 aa, synthetic construct
SEVEFSHEYWMRHALTLAKRARDEGEVPVGAVLVLNNRVIGEGWNRAIGLYDPTAHAEIMALRQGGLVMQNYGLIDATLYSTFEPCVMCAGAMIHSRIGRVVFGVRNSKRGAAGSLMNVLNYPGMDHRVEITEGILADECAALLCDFYRMPRRVFNAQKKAQSSIN

SEQ ID NO: 124 - DNA, 23 bp, synthetic construct
  source 1..23: mol_type = other DNA
agacttctcctcaggagtcagat

SEQ ID NO: 125 - DNA, 23 bp, synthetic construct
  source 1..23: mol_type = other DNA
ttacttctccacagcagtcaggt

SEQ ID NO: 126 - DNA, 23 bp, synthetic construct
  source 1..23: mol_type = other DNA
aaacttctccactggagtcaggg

SEQ ID NO: 127 - DNA, 23 bp, synthetic construct
  source 1..23: mol_type = other DNA
gagcttcttcacaagagtcaggt

SEQ ID NO: 128 - DNA, 23 bp, synthetic construct
  source 1..23: mol_type = other DNA
ccctttctccacaggagtcagga

SEQ ID NO: 129 - DNA, 23 bp, synthetic construct
  source 1..23: mol_type = other DNA
tcacttctccagaggtgtcaggt

SEQ ID NO: 130 - DNA, 23 bp, synthetic construct
  source 1..23: mol_type = other DNA
ctgcttctccacaagagtcaggt

SEQ ID NO: 131 - DNA, 23 bp, synthetic construct
  source 1..23: mol_type = other DNA
aagtttctccagaggagtcaggt

SEQ ID NO: 132 - DNA, 23 bp, synthetic construct
  source 1..23: mol_type = other DNA
cttcttctccataggagtcagat

SEQ ID NO: 133 - DNA, 20 bp, synthetic construct
  source 1..20: mol_type = other DNA
ttctccacaggagtcaggtg

SEQ ID NO: 134-135 - skipped (2 entries)

SEQ ID NO: 136 - DNA, 20 bp, synthetic construct
  source 1..20: mol_type = other DNA
taatccacaggagtcaggtg

SEQ ID NO: 137 - DNA, 20 bp, synthetic construct
  source 1..20: mol_type = other DNA
ttctcctcaggagtcagatg

SEQ ID NO: 138 - DNA, 20 bp, synthetic construct
  source 1..20: mol_type = other DNA
ttctccacaggagtcaggag

SEQ ID NO: 139 - DNA, 20 bp, synthetic construct
  source 1..20: mol_type = other DNA
ttctccagaggagtcagggg

SEQ ID NO: 140 - DNA, 20 bp, synthetic construct
  source 1..20: mol_type = other DNA
ctccccacaggagtcaggtg

SEQ ID NO: 141 - DNA, 20 bp, synthetic construct
  source 1..20: mol_type = other DNA
ttctcctcaggagtcaggag

SEQ ID NO: 142 - protein, 7 aa, unidentified
  source 1..7: note = simian vacuolating virus 40
PKKKRKV

SEQ ID NO: 143 - protein, 19 aa, synthetic construct
MKRTADGSEFESPKKKRKV

SEQ ID NO: 144 - protein, 30 aa, synthetic construct
MDSLLMNRRKFLYQFKNVRWAKGRRETYLC

SEQ ID NO: 145 - protein, 20 aa, synthetic construct
AVKRPAATKKAGQAKKKKLD

SEQ ID NO: 146 - protein, 25 aa, synthetic construct
MSRRRKANPTKLSENAKKLAKEVEN

SEQ ID NO: 147 - protein, 9 aa, synthetic construct
PAAKRVKLD

SEQ ID NO: 148 - protein, 9 aa, synthetic construct
KLKIKRPVK

SEQ ID NO: 149 - protein, 8 aa, synthetic construct
VSRKRPRP

SEQ ID NO: 150 - protein, 10 aa, synthetic construct
EGAPPAKRAR

SEQ ID NO: 151 - protein, 12 aa, synthetic construct
PPQPKKKPLDGE

SEQ ID NO: 152 - protein, 21 aa, synthetic construct
SGGSKRTADGSEFEPKKKRKV

SEQ ID NO: 153 - protein, 18 aa, synthetic construct
KRTADGSEFESPKKKRKV

SEQ ID NO: 154 - protein, 16 aa, Xenopus laevis
KRXXXXXXXXXXKKKL

SEQ ID NO: 155 - protein, 17 aa, synthetic construct
KRTADGSEFEPKKKRKV

SEQ ID NO: 156 - protein, 150 aa, synthetic construct
  REGION 6..150: note = amino acids may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 157 - protein, 30 aa, synthetic construct
  REGION 2..30: note = amino acids may be absent
GGGGGGGGGGGGGGGGGGGGGGGGGGGGGG

SEQ ID NO: 158 - protein, 150 aa, synthetic construct
  REGION 6..150: note = amino acids may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 159 - protein, 90 aa, synthetic construct
  REGION 4..90: note = amino acids may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 160 - protein, 120 aa, synthetic construct
  REGION 5..120: note = amino acids may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 161 - protein, 60 aa, synthetic construct
  REGION 3..60: note = amino acids may be absent
XPXPXPXPXPXPXPXPXPXPXPXPXPXPXPXPXPXPXPXPXPXPXPXPXPXPXPXPXPXP

SEQ ID NO: 162 - protein, 16 aa, synthetic construct
SGSETPGTSESATPES

SEQ ID NO: 163 - protein, 12 aa, synthetic construct
SGSETPGTSESA

SEQ ID NO: 164 - protein, 21 aa, synthetic construct
SGSETPGTSESATPEGGSGGS

SEQ ID NO: 165 - protein, 32 aa, synthetic construct
SGGSSGGSSGSETPGTSESATPESSGGSSGGS

SEQ ID NO: 166 - protein, 10 aa, synthetic construct
SGGSGGSGGS

SEQ ID NO: 167 - protein, 60 aa, synthetic construct
SGGSSGGSSGSETPGTSESATPESAGSYPYDVPDYAGSAAPAAKKKKLDGSGSGGSSGGS

SEQ ID NO: 168 - protein, 6 aa, synthetic construct
GGSGGS

SEQ ID NO: 169 - protein, 9 aa, synthetic construct
GGSGGSGGS

SEQ ID NO: 170 - protein, 33 aa, synthetic construct
SGGSSGGSSGSETPGTSESATPESSGGSSGGSS

SEQ ID NO: 171 - protein, 60 aa, synthetic construct; identical to SEQ ID NO: 167

SEQ ID NO: 172 - DNA, 20 bp, synthetic construct
  source 1..20: mol_type = other DNA
ttctccccaggagccaggtg

SEQ ID NO: 173 - DNA, 20 bp, synthetic construct
  source 1..20: mol_type = other DNA
ttctgcacaggagtcatgtg

SEQ ID NO: 174 - DNA, 20 bp, synthetic construct
  source 1..20: mol_type = other DNA
ttctccccgggagtcaggtg

SEQ ID NO: 175 - DNA, 20 bp, synthetic construct
  source 1..20: mol_type = other DNA
ttctccagaggagtcaggtt

SEQ ID NO: 176 - DNA, 20 bp, synthetic construct
  source 1..20: mol_type = other DNA
ttctccataggagtcagatg

SEQ ID NO: 177 - protein, 229 aa, synthetic construct
MSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 178 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 179 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPRNRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 180 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 181 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 182 - protein, 229 aa, synthetic construct
MSSETGPVAVDPTLRRRIEPHEFEVFFDPRELAKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 183 - protein, 229 aa, synthetic construct
MSSETGPVAVDPTLRRRIEPHEFEVFFDPRELAAETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 184 - protein, 229 aa, synthetic construct
MSSETGPVAVDPTLRRRIEPHEFEVFFDPRELAAETCLLYEINWGGRHSIWRHTSQNTNKHVEVNFIEKFTTERYFCPNTRCSITWFLSWSPCGECSRAITEFLSRYPHVTLFIYIARLYHLANPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 185 - protein, 162 aa, synthetic construct
MFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYHEDERNRQGLRDLVNSGVTIRIMDLPDYNYCWKTFVSDQGGDEDYWPGHFAPWIKQYSLKL

SEQ ID NO: 186 - protein, 162 aa, synthetic construct
MFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 187 - DNA, 24 bp, synthetic construct
  source 1..24: mol_type = other DNA
gcgcaaagctgcatccaccccccg

SEQ ID NO: 188 - DNA, 24 bp, synthetic construct
  source 1..24: mol_type = other DNA
gaccagcccctcgaaggcaaggcc

SEQ ID NO: 189 - DNA, 23 bp, synthetic construct
  source 1..23: mol_type = other DNA
gcaagagcacaagaggaagagag

SEQ ID NO: 190 - DNA, 24 bp, synthetic construct
  source 1..24: mol_type = other DNA
gccctcatctccaatatggtatgg

SEQ ID NO: 191 - DNA, 24 bp, synthetic construct
  source 1..24: mol_type = other DNA
tatgttccagcttcctgggtctgc

SEQ ID NO: 192 - DNA, 24 bp, synthetic construct
  source 1..24: mol_type = other DNA
gaggcaagagggcggctttgggcg

SEQ ID NO: 193 - DNA, 23 bp, synthetic construct
  source 1..23: mol_type = other DNA
ggtccaaagcaggatgacaggca

SEQ ID NO: 194 - DNA, 24 bp, synthetic construct
  source 1..24: mol_type = other DNA
gttcgaggaccatgagatagaagg

SEQ ID NO: 195 - DNA, 23 bp, synthetic construct
  source 1..23: mol_type = other DNA
gccagggctggagaagcagaaaa

SEQ ID NO: 196 - DNA, 22 bp, synthetic construct
  source 1..22: mol_type = other DNA
ggctacagcaacagggtggtgg

SEQ ID NO: 197 - DNA, 22 bp, synthetic construct
  source 1..22: mol_type = other DNA
gagcacaagaggaagagagaga

SEQ ID NO: 198 - DNA, 24 bp, synthetic construct
  source 1..24: mol_type = other DNA
gtccccagcccaaaccgccgcggc

SEQ ID NO: 199 - DNA, 23 bp, synthetic construct
  source 1..23: mol_type = other DNA
gatatggtccagtagatattgaa

SEQ ID NO: 200 - DNA, 144 bp, synthetic construct
  source 1..144: mol_type = other DNA
gttcctcactactcagagccagagttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 201 - DNA, 144 bp, synthetic construct
  source 1..144: mol_type = other DNA
tgttcctcactactcagagccaggttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 202 - DNA, 144 bp, synthetic construct
  source 1..144: mol_type = other DNA
ttcctcactactcagagccagaggttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 203 - DNA, 144 bp, synthetic construct
  source 1..144: mol_type = other DNA
tgttccgccctactcagagccaggttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 204 - DNA, 141 bp, synthetic construct
  source 1..141: mol_type = other DNA
ggcccagactgagcacgtgagttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 205 - DNA, 144 bp, synthetic construct
  source 1..144: mol_type = other DNA
atggcccagactgagcacgtgaggttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 206 - protein, 38 aa, synthetic construct
NQSSNFGPMKGGNFGGRSSGPYGGGGQYFAKPRNQGGY

SEQ ID NO: 207 - DNA, 23 bp, synthetic construct
  source 1..23: mol_type = other DNA
gtctccgctttaacccccacctc

SEQ ID NO: 208 - DNA, 24 bp, synthetic construct
  source 1..24: mol_type = other DNA
gacccaggggtagaaatggagagg

SEQ ID NO: 209 - DNA, 22 bp, synthetic construct
  source 1..22: mol_type = other DNA
gaatggggccatgccgaccaaa

SEQ ID NO: 210 - DNA, 24 bp, synthetic construct
  source 1..24: mol_type = other DNA
gctaccagcccccactggccctca

SEQ ID NO: 211 - DNA, 23 bp, synthetic construct
  source 1..23: mol_type = other DNA
ggtgtgcagacggcagtcactag

SEQ ID NO: 212 - DNA, 22 bp, synthetic construct
  source 1..22: mol_type = other DNA
gccgaccaaagcgccgatggat

SEQ ID NO: 213 - DNA, 23 bp, synthetic construct
  source 1..23: mol_type = other DNA
gcggccggatatcagctgccacg

SEQ ID NO: 214 - protein, 5 aa, synthetic construct
ERYGE

SEQ ID NO: 215 - DNA, 23 bp, synthetic construct
  source 1..23: mol_type = other DNA
gccaagaagtcgcccatcgcagc

SEQ ID NO: 216 - DNA, 23 bp, synthetic construct
  source 1..23: mol_type = other DNA
gggtggggtcagacgtccaaaac

SEQ ID NO: 217 - DNA, 23 bp, synthetic construct
  source 1..23: mol_type = other DNA
gctcatctccaatatggtatggc

SEQ ID NO: 218 - DNA, 23 bp, synthetic construct
  source 1..23: mol_type = other DNA
gcacaaccagtggaggcaagagg

SEQ ID NO: 219 - DNA, 23 bp, synthetic construct
  source 1..23: mol_type = other DNA
gaaatggcacacacatccctcgt

SEQ ID NO: 220 - DNA, 23 bp, synthetic construct
  source 1..23: mol_type = other DNA
gctgcggaaccgcttcttcggcc

SEQ ID NO: 221 - DNA, 23 bp, synthetic construct
  source 1..23: mol_type = other DNA
ggcgcaaagcaggaggagtcagg

SEQ ID NO: 222 - DNA, 22 bp, synthetic construct
  source 1..22: mol_type = other DNA
ggaggatgaaacaatgtctttg

SEQ ID NO: 223 - DNA, 24 bp, synthetic construct
  source 1..24: mol_type = other DNA
gaggggagaagaaagagagatgta

SEQ ID NO: 224 - DNA, 23 bp, synthetic construct
  source 1..23: mol_type = other DNA
gcagagcaaataccagagataag

SEQ ID NO: 225 - DNA, 23 bp, synthetic construct
  source 1..23: mol_type = other DNA
gtgtggttccagaaccggaggac

SEQ ID NO: 226 - DNA, 24 bp, synthetic construct
  source 1..24: mol_type = other DNA
gttggctacagcaacagggtggtg

SEQ ID NO: 227 - DNA, 23 bp, synthetic construct
  source 1..23: mol_type = other DNA
gtggtggacctcatggcccacat

SEQ ID NO: 228 - DNA, 23 bp, synthetic construct
  source 1..23: mol_type = other DNA
ggaactggaccccgcccaaagcc

SEQ ID NO: 229 - DNA, 23 bp, synthetic construct
  source 1..23: mol_type = other DNA
gatatgggctatttaaaaatacg

SEQ ID NO: 230 - DNA, 23 bp, synthetic construct
  source 1..23: mol_type = other DNA
ggggtccagttccgggattagcg

SEQ ID NO: 231 - DNA, 24 bp, synthetic construct
  source 1..24: mol_type = other DNA
gtcagaaaaagggccctgacaact

SEQ ID NO: 232 - DNA, 24 bp, synthetic construct
  source 1..24: mol_type = other DNA
gcctgcaccccatgcctatgttcc

SEQ ID NO: 233 - DNA, 24 bp, synthetic construct
  source 1..24: mol_type = other DNA
gtccagcagaggggagaagaaaga

SEQ ID NO: 234 - DNA, 23 bp, synthetic construct
  source 1..23: mol_type = other DNA
atattggagatgagggtggcaag

SEQ ID NO: 235 - DNA, 24 bp, synthetic construct
  source 1..24: mol_type = other DNA
gcccagatagacaggagacagcgc

SEQ ID NO: 236 - DNA, 23 bp, synthetic construct
  source 1..23: mol_type = other DNA
gatcatggaagggccgccatacc

SEQ ID NO: 237 - DNA, 23 bp, synthetic construct
  source 1..23: mol_type = other DNA
gtgggctgtaacaggagggccag

SEQ ID NO: 238 - DNA, 23 bp, synthetic construct
  source 1..23: mol_type = other DNA
ggaaaaaggccccagaaggagtc

SEQ ID NO: 239 - DNA, 26 bp, synthetic construct
  source 1..26: mol_type = other DNA
gcagcaagagcacaagaggaagagag

SEQ ID NO: 240 - DNA, 25 bp, synthetic construct
  source 1..25: mol_type = other DNA
gagcaagagcacaagaggaagagag

SEQ ID NO: 241 - DNA, 24 bp, synthetic construct
  source 1..24: mol_type = other DNA
ggcaagagcacaagaggaagagag

SEQ ID NO: 242 - DNA, 22 bp, synthetic construct
  source 1..22: mol_type = other DNA
gaagagcacaagaggaagagag

SEQ ID NO: 243 - DNA, 21 bp, synthetic construct
  source 1..21: mol_type = other DNA
gagagcacaagaggaagagag

SEQ ID NO: 244 - DNA, 20 bp, synthetic construct
  source 1..20: mol_type = other DNA
ggagcacaagaggaagagag

SEQ ID NO: 245 - DNA, 26 bp, synthetic construct
  source 1..26: mol_type = other DNA
gtggaaatggcacacacatccctcgt

SEQ ID NO: 246 - DNA, 25 bp, synthetic construct
  source 1..25: mol_type = other DNA
gggaaatggcacacacatccctcgt

SEQ ID NO: 247 - DNA, 24 bp, synthetic construct
  source 1..24: mol_type = other DNA
ggaaatggcacacacatccctcgt

SEQ ID NO: 248 - DNA, 22 bp, synthetic construct
  source 1..22: mol_type = other DNA
gaatggcacacacatccctcgt

SEQ ID NO: 249 - DNA, 21 bp, synthetic construct
  source 1..21: mol_type = other DNA
gatggcacacacatccctcgt

SEQ ID NO: 250 - DNA, 20 bp, synthetic construct
  source 1..20: mol_type = other DNA
gtggcacacacatccctcgt

SEQ ID NO: 251 - DNA, 26 bp, synthetic construct
  source 1..26: mol_type = other DNA
gagggtccaaagcaggatgacaggca

SEQ ID NO: 252 - DNA, 25 bp, synthetic construct
  source 1..25: mol_type = other DNA
ggggtccaaagcaggatgacaggca

SEQ ID NO: 253 - DNA, 24 bp, synthetic construct
  source 1..24: mol_type = other DNA
gggtccaaagcaggatgacaggca

SEQ ID NO: 254 - DNA, 22 bp, synthetic construct
  source 1..22: mol_type = other DNA
gtccaaagcaggatgacaggca

SEQ ID NO: 255 - DNA, 21 bp, synthetic construct
  source 1..21: mol_type = other DNA
gccaaagcaggatgacaggca

SEQ ID NO: 256 - DNA, 20 bp, synthetic construct
  source 1..20: mol_type = other DNA
gcaaagcaggatgacaggca

SEQ ID NO: 257 - DNA, 26 bp, synthetic construct
  source 1..26: mol_type = other DNA
gatttggctacagcaacagggtggtg

SEQ ID NO: 258 - DNA, 25 bp, synthetic construct
  source 1..25: mol_type = other DNA
gtttggctacagcaacagggtggtg

SEQ ID NO: 259 - DNA, 23 bp, synthetic construct
  source 1..23: mol_type = other DNA
gtggctacagcaacagggtggtg

SEQ ID NO: 260 - DNA, 22 bp, synthetic construct
  source 1..22: mol_type = other DNA
gggctacagcaacagggtggtg

SEQ ID NO: 261 - DNA, 21 bp, synthetic construct
  source 1..21: mol_type = other DNA
ggctacagcaacagggtggtg

SEQ ID NO: 262 - DNA, 20 bp, synthetic construct
  source 1..20: mol_type = other DNA
gctacagcaacagggtggtg

SEQ ID NO: 263 - DNA, 26 bp, synthetic construct
  source 1..26: mol_type = other DNA
gccatattggagatgagggtggcaag

SEQ ID NO: 264 - DNA, 25 bp, synthetic construct
  source 1..25: mol_type = other DNA
gcatattggagatgagggtggcaag

SEQ ID NO: 265 - DNA, 24 bp, synthetic construct
  source 1..24: mol_type = other DNA
gatattggagatgagggtggcaag

SEQ ID NO: 266 - DNA, 22 bp, synthetic construct
  source 1..22: mol_type = other DNA
gattggagatgagggtggcaag

SEQ ID NO: 267 - DNA, 21 bp, synthetic construct
  source 1..21: mol_type = other DNA
gttggagatgagggtggcaag

SEQ ID NO: 268 - DNA, 20 bp, synthetic construct
  source 1..20: mol_type = other DNA
gtggagatgagggtggcaag

SEQ ID NO: 269 - DNA, 26 bp, synthetic construct
  source 1..26: mol_type = other DNA
ggggtgggctgtaacaggagggccag

SEQ ID NO: 270 - DNA, 25 bp, synthetic construct
  source 1..25: mol_type = other DNA
gggtgggctgtaacaggagggccag

SEQ ID NO: 271 - DNA, 24 bp, synthetic construct
  source 1..24: mol_type = other DNA
ggtgggctgtaacaggagggccag

SEQ ID NO: 272 - DNA, 22 bp, synthetic construct
  source 1..22: mol_type = other DNA
ggggctgtaacaggagggccag

SEQ ID NO: 273 - DNA, 21 bp, synthetic construct
  source 1..21: mol_type = other DNA
gggctgtaacaggagggccag

SEQ ID NO: 274 - DNA, 20 bp, synthetic construct
  source 1..20: mol_type = other DNA
ggctgtaacaggagggccag

SEQ ID NO: 275 - DNA, 20 bp, synthetic construct
  source 1..20: mol_type = other DNA
gctgcatccaccccccgagg

SEQ ID NO: 276 - DNA, 20 bp, synthetic construct
  source 1..20: mol_type = other DNA
gcacaagaggaagagagaga

SEQ ID NO: 277 - DNA, 20 bp, synthetic construct
  source 1..20: mol_type = other DNA
gtacttcgaaaaggtaggcg

SEQ ID NO: 278 - DNA, 21 bp, synthetic construct
  source 1..21: mol_type = other DNA
gaagcaggatgacaggcaggg

SEQ ID NO: 279 - DNA, 20 bp, synthetic construct
  source 1..20: mol_type = other DNA
gaccatgagatagaaggcgc

SEQ ID NO: 280 - DNA, 20 bp, synthetic construct
  source 1..20: mol_type = other DNA
gctggagaagcagaaaaaaa

SEQ ID NO: 281 - DNA, 21 bp, synthetic construct
  source 1..21: mol_type = other DNA
gcagcaacagggtggtggacc

SEQ ID NO: 282 - DNA, 21 bp, synthetic construct
  source 1..21: mol_type = other DNA
gcaagaggaagagagagaccc

SEQ ID NO: 283 - DNA, 20 bp, synthetic construct
  source 1..20: mol_type = other DNA
gctttaacccccacctccag

SEQ ID NO: 284 - DNA, 21 bp, synthetic construct
  source 1..21: mol_type = other DNA
gcagacggcagtcactagggg

SEQ ID NO: 285 - DNA, 21 bp, synthetic construct
  source 1..21: mol_type = other DNA
gctccaatatggtatggcggc

SEQ ID NO: 286 - DNA, 21 bp, synthetic construct
  source 1..21: mol_type = other DNA
gccagtggaggcaagagggcg

SEQ ID NO: 287 - DNA, 20 bp, synthetic construct
  source 1..20: mol_type = other DNA
gcacacacatccctcgtgca

SEQ ID NO: 288 - DNA, 21 bp, synthetic construct
  source 1..21: mol_type = other DNA
gaagcaggaggagtcagggct

SEQ ID NO: 289 - DNA, 20 bp, synthetic construct
  source 1..20: mol_type = other DNA
gaagaaagagagatgtaggg

SEQ ID NO: 290 - DNA, 21 bp, synthetic construct
  source 1..21: mol_type = other DNA
gcaaataccagagataagaga

SEQ ID NO: 291 - DNA, 21 bp, synthetic construct
  source 1..21: mol_type = other DNA
gacagcaacagggtggtggac

SEQ ID NO: 292 - DNA, 21 bp, synthetic construct
  source 1..21: mol_type = other DNA
gagaggggagaagaaagagag

SEQ ID NO: 293 - DNA, 20 bp, synthetic construct
  source 1..20: mol_type = other DNA
gagatgagggtggcaaggcg

SEQ ID NO: 294 - DNA, 21 bp, synthetic construct
  source 1..21: mol_type = other DNA
gtagacaggagacagcgctgg

SEQ ID NO: 295 - DNA, 21 bp, synthetic construct
  source 1..21: mol_type = other DNA
gtgtaacaggagggccaggag

SEQ ID NO: 296 - DNA, 21 bp, synthetic construct
  source 1..21: mol_type = other DNA
gaggccccagaaggagtctgg

SEQ ID NO: 297 - protein, 5 aa, synthetic construct
ERYGK

SEQ ID NO: 298 - protein, 5 aa, synthetic construct
VRYGK

SEQ ID NO: 299 - protein, 5 aa, synthetic construct
DRHGK

SEQ ID NO: 300 - protein, 5 aa, synthetic construct
DRYGK

SEQ ID NO: 301 - DNA, 20 bp, synthetic construct
  source 1..20: mol_type = other DNA
gcaaagcaggaggagtcagg

SEQ ID NO: 302 - DNA, 21 bp, synthetic construct
  source 1..21: mol_type = other DNA
gagggctggagaagcagaaaa

SEQ ID NO: 303 - DNA, 21 bp, synthetic construct
  source 1..21: mol_type = other DNA
gtccgctttaacccccacctc

SEQ ID NO: 304 - DNA, 21 bp, synthetic construct
  source 1..21: mol_type = other DNA
gtcatctccaatatggtatgg

SEQ ID NO: 305 - DNA, 23 bp, synthetic construct
  source 1..23: mol_type = other DNA
gcaatggccgggactcctgggag

SEQ ID NO: 306 - DNA, 24 bp, synthetic construct
  source 1..24: mol_type = other DNA
gaatggccgggactcctgggagca

SEQ ID NO: 307 - DNA, 24 bp, synthetic construct
  source 1..24: mol_type = other DNA
gatggccgggactcctgggagcac

SEQ ID NO: 308 - DNA, 20 bp, synthetic construct
  source 1..20: mol_type = other DNA
gccgggactcctgggagcac

SEQ ID NO: 309 - DNA, 21 bp, synthetic construct
  source 1..21: mol_type = other DNA
gccgggactcctgggagcact

SEQ ID NO: 310 - DNA, 21 bp, synthetic construct
  source 1..21: mol_type = other DNA
gcgggactcctgggagcactc

SEQ ID NO: 311 - DNA, 20 bp, synthetic construct
  source 1..20: mol_type = other DNA
gggactcctgggagcactct

SEQ ID NO: 312 - protein, 1567 aa, synthetic construct
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSAAFKSNPINYILGLAIGIASVGWAMVEIDEEGNPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIAKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPSSKKQRILLQKFDEDGFKECNLNDTRYVNRFLCQFVADHILLTGKGKRRVVASNGQITNLLRGFWRLRKVRAENDRHHALDAVVVACSTVAMQQKITRFVRYKEMNAFDGKTVDKETGKVLYQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVRSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 313 - protein, 1567 aa, synthetic construct
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSAAFKPNPINYILGLAIGIASVGWAMVEIDEEENPIRLIDLGVRVFKRAEVPKTGDSLAMARRLARSMRRLTRRRAHRLLRARRLLKREGVLQAADFDENGLIKSLPNTPWQLRAAALDRKLAPLEWSAVLLHLIKHRGYLSQRKNEGETAGKKLGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDIAGRLKDRVQPEILEALLKNISFDKFVQISLKSLRRIVPLMEQGKRYDEACAEIYGDRYGKKNTEAKIYLPPIPADEIRNPVVLRALSQTRKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNPREWQEFKARVETSRFPRSKKQRILLQKFDEDGFKECNLNDTRYVSRFLCQFVADHILLTGKGKRRVFASNGQITNLLRGFWGLRKVRAENARHHALDAVVVACSTVAMQQKITRFVRYKEMNAFDGKTIDKETGKALYQKTRFPQPWEFFAQEVMIRVFGKPDGKPEFEEADTPEKLRTLLAEKLSSRPEAAHEYVTPLFVSRAPNRKMSGAHKATLRSAKRFVKHNEKVSVKRVLLTEIKLADLENMVNYKNGREIELYEALKARLEAYGGNAKQAFDPKDNPFYKKGGQLVKAVRVEKTQESGVLLNKKNAYTIADNGDRVRVDVFCKVDKKGKNQYFIVPIYAWQVAENILPDIDCKGYRIDDSYTFCFSLHRYDLIAFQKDEKSKVEFAYYINCNASNGYFYLAWHDKGSKEQQFSISTQNLVLIQKYQVSELGKEIRPCRLKKRPPVRSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 314 - protein, 1567 aa, synthetic construct
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSAAFKPNPINYILGLAIGIASVGWAMVEIDEEENPIRLIDLGVRVFKRAEVPKTGDSLAMARKLARSMRRLTRRRAHRLLRARRLLKREGVLQAADFDENGLIKSLPNTPWQLRATALDRKLAPLEWSAVLLHLIKHRGYLSQRKNEGETANKKLGALLKGVANNAHALQTGDFRTPAELALNKFEKESGHIRNQRGDYSHTFSRKDLQAELILLFEKQKDFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDIAGRLKDRVQPEILEALLKHISFDKFVQISLKALRRIVPLMEQGKRYDEACAEIYGDRYGKKNTEKKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVSRFLCQFVADHILLTGKGKRRVFASNGQITNLLRGFWGLRKVRAENARHHSLDAVVVACSTVAMQQKITRFVRYKEMNAFDGKTIDKETGKVLHQRTHFPQPWEFFAQEVMIRVFGKPDGKPEFEEADTPEKLRTLLAEKLSIRPEAVHEYVTPLFVSRAPNRKMSGAHKATLRSAKRFVKHNEKISVKRVWLTEIKLADLENMVNYKNGREIELYEALKARLEAYGGNAKQAFDPKDNPFYKKGGQLVKAVRVEKTQKSGVLLNKRNAYTIADNGDRVRVDVFCKVDKKGKNQYFIVPIYAWQVAENILPDIDCKGYRIDDSYTFCFSLHRYDLIAFQKDEKSKVEFAYYINCNASNGNFYLAWHDKGSKEQQFCISTQNLVLIQKYQVNELGKEIRPCRMKKRPPVRSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 315 - protein, 1567 aa, synthetic construct
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVRSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 316 - protein, 1319 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSAAFKSNPINYILGLAIGIASVGWAMVEIDEEGNPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIAKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPSSKKQRILLQKFDEDGFKECNLNDTRYVNRFLCQFVADHILLTGKGKRRVVASNGQITNLLRGFWRLRKVRAENDRHHALDAVVVACSTVAMQQKITRFVRYKEMNAFDGKTVDKETGKVLYQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVRSGGSKRTADGSEFEPKKKRKV

SEQ ID NO: 317 - protein, 1319 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSAAFKPNPINYILGLDIGIASVGWAMVEIDEEENPIRLIDLGVRVFQRAEVPKTGDSLAMARRLARSVRRLTRRRAHRLLRARRLLKREGVLQAADFDENGLIKSLPHTPWQLRTAALDRKLTPLEWSAVLLHLIKHRGYLSQRKNEGETADKKLGALLKGVANNAHALQTGDFRTPAELALNKFEKESGHIRNQRGDYSHTFSRKNLQSGLILLFEKQKEFGNPHVSGGLKEGIETLLMTQRPALSGDAVQKMLGHCTFESAEPKAAKNTYTAERFIWLTKLNNLRILEQGSERPLTDTERATLMDEPYRKSKLTYAQARKLLGLEDTAFFKGLRYVKDNAEVSTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVRYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEGGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKEAGKALYQKARFPQPWEFFAQEVMIRVFGKPDGKPEFEEADTPEKLRTLLAERLSSRPEAVHEYVTPLFVSRAPNRKMSGAHKVILRSAKRFVKHNEKISVKRVWLTEIKLADLENMVNYRNGREIELYEALKARLEAYGGNAKQAFDPKDNPFYKKGGQLVKAVRVERTQERGVLLNKKNAYTIADNGDMVRVDVFCKVGKKGKNQYFIVPIYAWQVAENILPDIDCKGYRIDDSYTFCFSLHKYDLIAFQKDEKSKVEFAYYSYCHSSSGDFYLAWHDKGSKEQQFRISTQNLVLIQKYQVNELGKEIRPCRLKKRPLVRSGGSKRTADGSEFEPKKKRKV

SEQ ID NO: 318 - protein, 1319 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSAAFKPNPINYILGLAIGIASVGWAMVEIDEEENPIRLIDLGVRVFKRAEVPKTGDSLAMARRLARSMRRLTRRRAHRLLRARRLLKREGVLQAADFDENGLIKSLPNTPWQLRAAALDRKLAPLEWSAVLLHLIKHRGYLSQRKNEGETAGKKLGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDIAGRLKDRVQPEILEALLKNISFDKFVQISLKSLRRIVPLMEQGKRYDEACAEIYGDRYGKKNTEAKIYLPPIPADEIRNPVVLRALSQTRKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNPREWQEFKARVETSRFPRSKKQRILLQKFDEDGFKECNLNDTRYVSRFLCQFVADHILLTGKGKRRVFASNGQITNLLRGFWGLRKVRAENARHHALDAVVVACSTVAMQQKITRFVRYKEMNAFDGKTIDKETGKALYQKTRFPQPWEFFAQEVMIRVFGKPDGKPEFEEADTPEKLRTLLAEKLSSRPEAAHEYVTPLFVSRAPNRKMSGAHKATLRSAKRFVKHNEKVSVKRVLLTEIKLADLENMVNYKNGREIELYEALKARLEAYGGNAKQAFDPKDNPFYKKGGQLVKAVRVEKTQESGVLLNKKNAYTIADNGDRVRVDVFCKVDKKGKNQYFIVPIYAWQVAENILPDIDCKGYRIDDSYTFCFSLHRYDLIAFQKDEKSKVEFAYYINCNASNGYFYLAWHDKGSKEQQFSISTQNLVLIQKYQVSELGKEIRPCRLKKRPPVRSGGSKRTADGSEFEPKKKRKV

SEQ ID NO: 319 - protein, 1319 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSAAFKPNPINYILGLAIGIASVGWAMVEIDEEENPIRLIDLGVRVFKRAEVPKTGDSLAMARKLARSMRRLTRRRAHRLLRARRLLKREGVLQAADFDENGLIKSLPNTPWQLRATALDRKLAPLEWSAVLLHLIKHRGYLSQRKNEGETANKKLGALLKGVANNAHALQTGDFRTPAELALNKFEKESGHIRNQRGDYSHTFSRKDLQAELILLFEKQKDFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDIAGRLKDRVQPEILEALLKHISFDKFVQISLKALRRIVPLMEQGKRYDEACAEIYGDRYGKKNTEKKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVSRFLCQFVADHILLTGKGKRRVFASNGQITNLLRGFWGLRKVRAENARHHSLDAVVVACSTVAMQQKITRFVRYKEMNAFDGKTIDKETGKVLHQRTHFPQPWEFFAQEVMIRVFGKPDGKPEFEEADTPEKLRTLLAEKLSIRPEAVHEYVTPLFVSRAPNRKMSGAHKATLRSAKRFVKHNEKISVKRVWLTEIKLADLENMVNYKNGREIELYEALKARLEAYGGNAKQAFDPKDNPFYKKGGQLVKAVRVEKTQKSGVLLNKRNAYTIADNGDRVRVDVFCKVDKKGKNQYFIVPIYAWQVAENILPDIDCKGYRIDDSYTFCFSLHRYDLIAFQKDEKSKVEFAYYINCNASNGNFYLAWHDKGSKEQQFCISTQNLVLIQKYQVNELGKEIRPCRMKKRPPVRSGGSKRTADGSEFEPKKKRKV

SEQ ID NO: 320 - protein, 1319 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLITSLPNTPWQLRAAALDRKLTPLEWSAVLLHLIKHRGYLSQRKNEGETAAKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTLEPTEPKAAKNTYTAERFIWLTKLNNLRILEQGSERPLTDTERSTLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGVHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQKSGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSSGGFYLAWHDKGSREQRFRISTQNLALIQKYQVNELGKEIRPCRLKKRPPVRSGGSKRTADGSEFEPKKKRKV

SEQ ID NO: 321 - DNA, 29 bp, synthetic construct
  source 1..29: mol_type = other DNA
ggcgcaaagcaggaggagtcaggnnnnnn

SEQ ID NO: 322 - DNA, 29 bp, synthetic construct
  source 1..29: mol_type = other DNA
ggcgcaaagcaggaggagtcagggcttct

SEQ ID NO: 323 - DNA, 29 bp, synthetic construct
  source 1..29: mol_type = other DNA
ggcacaaagcaaaaggagtcaggcaggct

SEQ ID NO: 324 - DNA, 29 bp, synthetic construct
  source 1..29: mol_type = other DNA
aggacagttgaggatgtgacatggaatac

SEQ ID NO: 325 - DNA, 29 bp, synthetic construct
  source 1..29: mol_type = other DNA
ggagggaggaaggaagggagggaggaagg

SEQ ID NO: 326 - DNA, 29 bp, synthetic construct
  source 1..29: mol_type = other DNA
ggcataatgccagggggctgagttgggaa

SEQ ID NO: 327 - DNA, 29 bp, synthetic construct
  source 1..29: mol_type = other DNA
tgggggtactaggaggagtggggggcact

SEQ ID NO: 328 - DNA, 29 bp, synthetic construct
  source 1..29: mol_type = other DNA
agaagaaggtacaaggagtccctgcagca

SEQ ID NO: 329 - DNA, 29 bp, synthetic construct
  source 1..29: mol_type = other DNA
cacccgaggctgggggaagcaagggagct

SEQ ID NO: 330 - DNA, 29 bp, synthetic construct
  source 1..29: mol_type = other DNA
ggtgtcgtggtgggggaggaagttgtggt

SEQ ID NO: 331 - DNA, 29 bp, synthetic construct
  source 1..29: mol_type = other DNA
ataggagagcaggaggagtcagggaggcc

SEQ ID NO: 332 - DNA, 29 bp, synthetic construct
  source 1..29: mol_type = other DNA
tgagacagaaatgaggactcactgtacac

SEQ ID NO: 333 - DNA, 125 bp, synthetic construct
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 334 - DNA, 121 bp, synthetic construct
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 335 - DNA, 109 bp, synthetic construct
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 336 - DNA, 102 bp, synthetic construct
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 337 - DNA, 87 bp, synthetic construct
  source 1..87: mol_type = other DNA
nnnnnnnnnnnnnnnnnnngttttagagctagaaatagcaagttaaaataaggctagtccgttatcaacttgaaaaagtgttttttt

SEQ ID NO: 338 - DNA, 76 bp, synthetic construct
  source 1..76: mol_type = other DNA
nnnnnnnnnnnnnnnnnnnngttttagagctagaaatagcaagttaaaataaggctagtccgttatcatttttttt

SEQ ID NO: 339 - RNA, 81 nt, synthetic construct
  source 1..81: mol_type = other RNA
gttttagagctagaaatagcaagttaaaataaggctagtccgttatcaacttgaaaaagtggcaccgagtcggtgcttttt

SEQ ID NO: 340 - DNA, 29 bp, synthetic construct
  source 1..29: mol_type = other DNA
ttccccaagccacaggcgccactattcta

SEQ ID NO: 341 - DNA, 29 bp, synthetic construct
  source 1..29: mol_type = other DNA
ggtgaaaagctggggagggaatgatccag

SEQ ID NO: 342 - DNA, 29 bp, synthetic construct
  source 1..29: mol_type = other DNA
gctactcgggaggctgaggcaggagaatc

SEQ ID NO: 343 - DNA, 29 bp, synthetic construct
  source 1..29: mol_type = other DNA
tgcaaaagacaggaggagtcagggaagat

SEQ ID NO: 344 - DNA, 23 bp, synthetic construct
  source 1..23: mol_type = other DNA
ggagagcaggaggagtcaggnnn

SEQ ID NO: 345 - DNA, 23 bp, synthetic construct
  source 1..23: mol_type = other DNA
ggagagcaggaggagtcagggag

SEQ ID NO: 346 - DNA, 23 bp, synthetic construct
  source 1..23: mol_type = other DNA
gcaaagcaggaggagtcagggct

SEQ ID NO: 347 - DNA, 23 bp, synthetic construct
  source 1..23: mol_type = other DNA
gtaaagcaggaggagtcaggaag

SEQ ID NO: 348 - DNA, 23 bp, synthetic construct
  source 1..23: mol_type = other DNA
ccagagcaggaggagtcaggaca

SEQ ID NO: 349 - DNA, 23 bp, synthetic construct
  source 1..23: mol_type = other DNA
ctgaagcaggaagagtcagggaa

SEQ ID NO: 350 - DNA, 23 bp, synthetic construct
  source 1..23: mol_type = other DNA
ttaaagcaggaggagtcagtgca

SEQ ID NO: 351 - DNA, 23 bp, synthetic construct
  source 1..23: mol_type = other DNA
aaggagcaggaggagtcagggac

SEQ ID NO: 352 - DNA, 23 bp, synthetic construct
  source 1..23: mol_type = other DNA
acaaagcaaaaggagtcaggcag

SEQ ID NO: 353 - DNA, 23 bp, synthetic construct
  source 1..23: mol_type = other DNA
ccaaaacaggaggagtcagacag

SEQ ID NO: 354 - DNA, 23 bp, synthetic construct
  source 1..23: mol_type = other DNA
atgaagcaggaggagtcagagaa

SEQ ID NO: 355 - DNA, 23 bp, synthetic construct
  source 1..23: mol_type = other DNA
gaccagcaggaggagtcaggaga

SEQ ID NO: 356 - DNA, 23 bp, synthetic construct
  source 1..23: mol_type = other DNA
caaagtcaggaggaatcagggag

SEQ ID NO: 357 - DNA, 23 bp, synthetic construct
  source 1..23: mol_type = other DNA
aaaagacaggaggagtcagggaa

SEQ ID NO: 358 - DNA, 23 bp, synthetic construct
  source 1..23: mol_type = other DNA
gcaaagctggaggaatcaggaag

SEQ ID NO: 359 - DNA, 23 bp, synthetic construct
  source 1..23: mol_type = other DNA
gcagagcagaaggaggcagggaa

SEQ ID NO: 360 - DNA, 23 bp, synthetic construct
  source 1..23: mol_type = other DNA
gggaggcaggaggagtcagggaa

SEQ ID NO: 361 - DNA, 23 bp, synthetic construct
  source 1..23: mol_type = other DNA
acaaggcaagaagagtcagggac

SEQ ID NO: 362 - DNA, 23 bp, synthetic construct
  source 1..23: mol_type = other DNA
gcagaggaggaggagtcaaggga

SEQ ID NO: 363 - DNA, 23 bp, synthetic construct
  source 1..23: mol_type = other DNA
acaaagcaggaggaggcaggaac

SEQ ID NO: 364 - DNA, 23 bp, synthetic construct
  source 1..23: mol_type = other DNA
ggaaagcagtaggagtcagagag

SEQ ID NO: 365 - DNA, 23 bp, synthetic construct
  source 1..23: mol_type = other DNA
gaaaagcaggaggaggcagggaa

SEQ ID NO: 366 - DNA, 23 bp, synthetic construct
  source 1..23: mol_type = other DNA
gccaagcatgaggagtcaggggt

SEQ ID NO: 367 - DNA, 23 bp, synthetic construct
  source 1..23: mol_type = other DNA
ccaaggcaggaggaggcaggagg

SEQ ID NO: 368 - DNA, 23 bp, synthetic construct
  source 1..23: mol_type = other DNA
cacacgcaggaggagtcagtggt

SEQ ID NO: 369 - DNA, 23 bp, synthetic construct
  source 1..23: mol_type = other DNA
aaaaagcaaaaggagtcaggaga

SEQ ID NO: 370 - DNA, 23 bp, synthetic construct
  source 1..23: mol_type = other DNA
agacagcaggaggagtcaggggt

SEQ ID NO: 371 - DNA, 23 bp, synthetic construct
  source 1..23: mol_type = other DNA
gcatagctggaggaatcagggaa

SEQ ID NO: 372 - DNA, 23 bp, synthetic construct
  source 1..23: mol_type = other DNA
gcaatgcaggaggaatcaggcaa

SEQ ID NO: 373 - DNA, 23 bp, synthetic construct
  source 1..23: mol_type = other DNA
gcaaagctggaggagtcaggaca

SEQ ID NO: 374 - DNA, 23 bp, synthetic construct
  source 1..23: mol_type = other DNA
aggaagcagggggagtcaggaaa

SEQ ID NO: 375 - DNA, 23 bp, synthetic construct
  source 1..23: mol_type = other DNA
taaaaggaggaggagtcagagat

SEQ ID NO: 376 - DNA, 23 bp, synthetic construct
  source 1..23: mol_type = other DNA
tcaaagcaggaggagccaggagt

SEQ ID NO: 377 - DNA, 23 bp, synthetic construct
  source 1..23: mol_type = other DNA
ggtacaaagcaggaggcagggaa

SEQ ID NO: 378 - DNA, 23 bp, synthetic construct
  source 1..23: mol_type = other DNA
aaaaagcaagaggagtcaagggg

SEQ ID NO: 379 - DNA, 23 bp, synthetic construct
  source 1..23: mol_type = other DNA
ggcaaaggggaggagtccagggg

SEQ ID NO: 380 - DNA, 23 bp, synthetic construct
  source 1..23: mol_type = other DNA
gcggagccggaggagtcagggac

SEQ ID NO: 381 - DNA, 23 bp, synthetic construct
  source 1..23: mol_type = other DNA
tccaagcagcaggagtcaggtac

SEQ ID NO: 382 - DNA, 23 bp, synthetic construct
  source 1..23: mol_type = other DNA
gctgagcagtaggagtcaggcag

SEQ ID NO: 383 - DNA, 23 bp, synthetic construct
  source 1..23: mol_type = other DNA
tgagcgcaggaggagtccagggc

SEQ ID NO: 384 - DNA, 23 bp, synthetic construct
  source 1..23: mol_type = other DNA
gcagggcaggaggagacaggaag

SEQ ID NO: 385 - DNA, 23 bp, synthetic construct
  source 1..23: mol_type = other DNA
gcataccaggaggaggcaggaag

SEQ ID NO: 386 - DNA, 23 bp, synthetic construct
  source 1..23: mol_type = other DNA
gctaggcaggaggaatcaggagt

SEQ ID NO: 387 - DNA, 23 bp, synthetic construct
  source 1..23: mol_type = other DNA
acaaagctggaagagtcagaaag

SEQ ID NO: 388 - DNA, 23 bp, synthetic construct
  source 1..23: mol_type = other DNA
aataaacaggaagagtcaggtgg

SEQ ID NO: 389 - DNA, 23 bp, synthetic construct
  source 1..23: mol_type = other DNA
gcagagcaggaggcagggaggct

SEQ ID NO: 390 - DNA, 23 bp, synthetic construct
  source 1..23: mol_type = other DNA
gcagggcaggaggaggcaggtgg

SEQ ID NO: 391 - DNA, 23 bp, synthetic construct
  source 1..23: mol_type = other DNA
gcacagcaggagaagtcagtaaa

SEQ ID NO: 392 - DNA, 23 bp, synthetic construct
  source 1..23: mol_type = other DNA
aggatgcaggaggagtcagggaa

SEQ ID NO: 393 - DNA, 23 bp, synthetic construct
  source 1..23: mol_type = other DNA
acagaacaggaggagacagacag

SEQ ID NO: 394 - DNA, 23 bp, synthetic construct
  source 1..23: mol_type = other DNA
gcaaagcgggaggcagggaggct

SEQ ID NO: 395 - DNA, 23 bp, synthetic construct
  source 1..23: mol_type = other DNA
gaagagcaggaggagacggggta

SEQ ID NO: 396 - DNA, 29 bp, synthetic construct
  source 1..29: mol_type = other DNA
gccagggctggagaagcagaaaannnnnn

SEQ ID NO: 397 - DNA, 29 bp, synthetic construct
  source 1..29: mol_type = other DNA
gccagggctggagaagcagaaaaaaagca

SEQ ID NO: 398 - DNA, 29 bp, synthetic construct
  source 1..29: mol_type = other DNA
gacagagacagagagagagagaggaacag

SEQ ID NO: 399 - DNA, 29 bp, synthetic construct
  source 1..29: mol_type = other DNA
acacggcctggaggtactggggatcgaac

SEQ ID NO: 400 - DNA, 29 bp, synthetic construct
  source 1..29: mol_type = other DNA
ccttgttgtctagaaggagggaattttac

SEQ ID NO: 401 - DNA, 29 bp, synthetic construct
  source 1..29: mol_type = other DNA
actcgggaggctgaggcaggacaattgct

SEQ ID NO: 402 - DNA, 29 bp, synthetic construct
  source 1..29: mol_type = other DNA
gtcaatggtagaaaaggaaatatcttcgt

SEQ ID NO: 403 - DNA, 29 bp, synthetic construct
  source 1..29: mol_type = other DNA
tagaagtatagagaaagagacagaagcaa

SEQ ID NO: 404 - DNA, 29 bp, synthetic construct
  source 1..29: mol_type = other DNA
tccagacatggaaactgggaggaaacaaa

SEQ ID NO: 405 - DNA, 29 bp, synthetic construct
  source 1..29: mol_type = other DNA
cccgcccccggacttgcaggcaaggtcct

SEQ ID NO: 406 - DNA, 29 bp, synthetic construct
  source 1..29: mol_type = other DNA
gaaagttacggagcccccgaaagaccggc

SEQ ID NO: 407 - DNA, 29 bp, synthetic construct
  source 1..29: mol_type = other DNA
gacagggagggagggacggagacaggcag

SEQ ID NO: 408 - DNA, 29 bp, synthetic construct
  source 1..29: mol_type = other DNA
ccgaggctgggggaagcaagggagctaca

SEQ ID NO: 409 - DNA, 29 bp, synthetic construct
  source 1..29: mol_type = other DNA
gagagagagagagagacagagagagacag

SEQ ID NO: 410 - DNA, 29 bp, synthetic construct
  source 1..29: mol_type = other DNA
gcatggatgagagaagcctggagacaggg

SEQ ID NO: 411 - DNA, 29 bp, synthetic construct
  source 1..29: mol_type = other DNA
gctagggctagggttagggttagggctag

SEQ ID NO: 412 - protein, 32 aa, synthetic construct; identical to SEQ ID NO: 165

SEQ ID NO: 413 - protein, 4 aa, synthetic construct
SGGS

SEQ ID NO: 414 - DNA, 29 bp, synthetic construct
  source 1..29: mol_type = other DNA
gggagcccctcaggagaagacacatttaa

SEQ ID NO: 415 - DNA, 29 bp, synthetic construct
  source 1..29: mol_type = other DNA
tccatgtctgtaaaatggggctagcaata

SEQ ID NO: 416 - DNA, 29 bp, synthetic construct
  source 1..29: mol_type = other DNA
gagatggctggaagtacaaatctaaactg

SEQ ID NO: 417 - DNA, 29 bp, synthetic construct
  source 1..29: mol_type = other DNA
ccaaattaccgagaaaaaaaaaatgttat

SEQ ID NO: 418 - DNA, 23 bp, synthetic construct
  source 1..23: mol_type = other DNA
agggctggagaagcagaaaannn

SEQ ID NO: 419 - DNA, 23 bp, synthetic construct
  source 1..23: mol_type = other DNA
agggctggagaagcagaaaaaaa

SEQ ID NO: 420 - DNA, 23 bp, synthetic construct
  source 1..23: mol_type = other DNA
agggctggagaagcagaaaatgg

SEQ ID NO: 421 - DNA, 23 bp, synthetic construct
  source 1..23: mol_type = other DNA
agggctggagaagcagaaaaggg

SEQ ID NO: 422 - protein, 16 aa, synthetic construct
SGSETPGTSESATPES

SEQ ID NO: 423 - DNA, 23 bp, synthetic construct
  source 1..23: mol_type = other DNA
agggctgaagaagcagaaacaag

SEQ ID NO: 424 - DNA, 23 bp, synthetic construct
  source 1..23: mol_type = other DNA
gcagctggagaagcagaaaactc

SEQ ID NO: 425 - DNA, 23 bp, synthetic construct
  source 1..23: mol_type = other DNA
ggtgctggagaagcagaaatgaa

SEQ ID NO: 426 - DNA, 23 bp, synthetic construct
  source 1..23: mol_type = other DNA
ggggctggagaggcagaaaagga

SEQ ID NO: 427 - DNA, 23 bp, synthetic construct
  source 1..23: mol_type = other DNA
gaggctgaagaagcagaaagagg

SEQ ID NO: 428 - DNA, 23 bp, synthetic construct
  source 1..23: mol_type = other DNA
taggctggagaaacagaaaatac

SEQ ID NO: 429 - DNA, 23 bp, synthetic construct
  source 1..23: mol_type = other DNA
caagctggagaagcagaaaaaga

SEQ ID NO: 430 - protein, 4 aa, synthetic construct
GGGS

SEQ ID NO: 431 - protein, 5 aa, synthetic construct
SGGGS

SEQ ID NO: 432 - DNA, 23 bp, synthetic construct
  source 1..23: mol_type = other DNA
aagggcagagaagcagaaaaggg

SEQ ID NO: 433 - DNA, 23 bp, synthetic construct
  source 1..23: mol_type = other DNA
agagggggagaagcagagaataa

SEQ ID NO: 434 - DNA, 23 bp, synthetic construct
  source 1..23: mol_type = other DNA
ggggatggagaagaagaaaataa

SEQ ID NO: 435 - DNA, 23 bp, synthetic construct
  source 1..23: mol_type = other DNA
agggcaggagaagaaaaggggta

SEQ ID NO: 436 - DNA, 23 bp, synthetic construct
  source 1..23: mol_type = other DNA
gagactggagaagcagaaaactg

SEQ ID NO: 437 - DNA, 23 bp, synthetic construct
  source 1..23: mol_type = other DNA
aggaagggagaagcagaaaacac

SEQ ID NO: 438 - DNA, 23 bp, synthetic construct
  source 1..23: mol_type = other DNA
cgaggaggagaagcagaaaagag

SEQ ID NO: 439 - DNA, 23 bp, synthetic construct
  source 1..23: mol_type = other DNA
agagggggagaagcagaaagcat

SEQ ID NO: 440 - DNA, 23 bp, synthetic construct
  source 1..23: mol_type = other DNA
taggctggagaagcagaatagag

SEQ ID NO: 441 - DNA, 23 bp, synthetic construct
  source 1..23: mol_type = other DNA
cagggctgggaagcagaaataat

SEQ ID NO: 442 - DNA, 23 bp, synthetic construct
  source 1..23: mol_type = other DNA
agggctaaagaagcagaaagaag

SEQ ID NO: 443 - DNA, 23 bp, synthetic construct
  source 1..23: mol_type = other DNA
gagggcagagaagcagaaaaaca

SEQ ID NO: 444 - DNA, 23 bp, synthetic construct
  source 1..23: mol_type = other DNA
ggggctggagaagcagaaaggga

SEQ ID NO: 445 - DNA, 23 bp, synthetic construct
  source 1..23: mol_type = other DNA
agggctgaagaagcagaaacaga

SEQ ID NO: 446 - DNA, 23 bp, synthetic construct
  source 1..23: mol_type = other DNA
caagctggagaagcagaaaagcc

SEQ ID NO: 447 - DNA, 23 bp, synthetic construct
  source 1..23: mol_type = other DNA
aaggcaggagaagcagaaataac

SEQ ID NO: 448 - DNA, 23 bp, synthetic construct
  source 1..23: mol_type = other DNA
ctggctgaagaagcagaaaagat

SEQ ID NO: 449 - DNA, 23 bp, synthetic construct
  source 1..23: mol_type = other DNA
agggaacaaaaagcagatgggtt

SEQ ID NO: 450 - DNA, 29 bp, synthetic construct
  source 1..29: mol_type = other DNA
gtctccgctttaacccccacctcnnnnnn

SEQ ID NO: 451 - DNA, 29 bp, synthetic construct
  source 1..29: mol_type = other DNA
gtctccgctttaacccccacctccagccg

SEQ ID NO: 452 - DNA, 29 bp, synthetic construct
  source 1..29: mol_type = other DNA
ggccccgcctcagcccccacctccagtcc

SEQ ID NO: 453 - DNA, 29 bp, synthetic construct
  source 1..29: mol_type = other DNA
ctcagccccctggcacccaactctacccc

SEQ ID NO: 454 - DNA, 29 bp, synthetic construct
  source 1..29: mol_type = other DNA
gcccctgctgtagactccctgtataaccc

SEQ ID NO: 455 - DNA, 29 bp, synthetic construct
  source 1..29: mol_type = other DNA
gcctgtgttccaaggccttgctcttgggt

SEQ ID NO: 456 - DNA, 29 bp, synthetic construct
  source 1..29: mol_type = other DNA
gtctcagattgccacctcccctacaattc

SEQ ID NO: 457 - DNA, 29 bp, synthetic construct
  source 1..29: mol_type = other DNA
gtgcaagcctgtaatcccagctactcagg

SEQ ID NO: 458 - DNA, 29 bp, synthetic construct
  source 1..29: mol_type = other DNA
gtatactattttacattcccaccaacagt

SEQ ID NO: 459 - DNA, 29 bp, synthetic construct
  source 1..29: mol_type = other DNA
accctaaccttaaccctaaccctaaccct

SEQ ID NO: 460 - DNA, 29 bp, synthetic construct
  source 1..29: mol_type = other DNA
accctaaccctaaccccaaccccaaaccg

SEQ ID NO: 461 - DNA, 29 bp, synthetic construct
  source 1..29: mol_type = other DNA
accttaaccctaaccctaaccctaaccct

SEQ ID NO: 462 - DNA, 29 bp, synthetic construct
  source 1..29: mol_type = other DNA
gccttcgttggaaacgggatttcttcata

SEQ ID NO: 463 - DNA, 29 bp, synthetic construct
  source 1..29: mol_type = other DNA
accctaaccctaaccctcaccctaaccct

SEQ ID NO: 464 - DNA, 29 bp, synthetic construct
  source 1..29: mol_type = other DNA
gcctcgcccttcctcccttccttccaccc

SEQ ID NO: 465 - DNA, 29 bp, synthetic construct
  source 1..29: mol_type = other DNA
ctccctgttccccactccaccccaataaa

SEQ ID NO: 466 - DNA, 29 bp, synthetic construct
  source 1..29: mol_type = other DNA
gtctccactcacacaaccttcccgagaca

SEQ ID NO: 467 - DNA, 29 bp, synthetic construct
  source 1..29: mol_type = other DNA
gaatccacttttgaaagttcctcaagcct

SEQ ID NO: 468 - DNA, 29 bp, synthetic construct
  source 1..29: mol_type = other DNA
gcccctccatgcagccccttcactggcca

SEQ ID NO: 469 - DNA, 29 bp, synthetic construct
  source 1..29: mol_type = other DNA
ctaacaaccctaaccctaaccctaaccct

SEQ ID NO: 470 - DNA, 29 bp, synthetic construct
  source 1..29: mol_type = other DNA
cactcccccagtgccccccactcctccta

SEQ ID NO: 471 - DNA, 29 bp, synthetic construct
  source 1..29: mol_type = other DNA
ctctgctgcttaacagccctgtggccatg

SEQ ID NO: 472 - DNA, 29 bp, synthetic construct
  source 1..29: mol_type = other DNA
aacacttttttatccatcagatcaattaa

SEQ ID NO: 473 - DNA, 29 bp, synthetic construct
  source 1..29: mol_type = other DNA
gtcatggcaacattcatcacctgcttgct

SEQ ID NO: 474 - DNA, 29 bp, synthetic construct
  source 1..29: mol_type = other DNA
ttgtccttttctttctccaccacaccctg

SEQ ID NO: 475 - DNA, 29 bp, synthetic construct
  source 1..29: mol_type = other DNA
accctagccctaaccctaaccctagccct

SEQ ID NO: 476 - DNA, 29 bp, synthetic construct
  source 1..29: mol_type = other DNA
gttgaggcttgaacatctagttcgacggg

SEQ ID NO: 477 - DNA, 29 bp, synthetic construct
  source 1..29: mol_type = other DNA
atagcagattaaacccaaatgtatcatat

SEQ ID NO: 478 - DNA, 29 bp, synthetic construct
  source 1..29: mol_type = other DNA
gacatcccctaatgccccagagcctgctg

SEQ ID NO: 479 - DNA, 29 bp, synthetic construct
  source 1..29: mol_type = other DNA
ttcccctctttaatctactgtgtggtttc

SEQ ID NO: 480 - DNA, 29 bp, synthetic construct
  source 1..29: mol_type = other DNA
ccctaacccttaacccctaacccctaacc

SEQ ID NO: 481 - DNA, 29 bp, synthetic construct
  source 1..29: mol_type = other DNA
accccaaccctaacccctaacccctaacc

SEQ ID NO: 482 - DNA, 29 bp, synthetic construct
  source 1..29: mol_type = other DNA
accgtaactctaaccctaaccccagccct

SEQ ID NO: 483 - DNA, 29 bp, synthetic construct
  source 1..29: mol_type = other DNA
tccttagtttcttcctcttcctcctgtac

SEQ ID NO: 484 - DNA, 29 bp, synthetic construct
  source 1..29: mol_type = other DNA
gtcaggagttcgacaccagcctgaccaat

SEQ ID NO: 485 - DNA, 29 bp, synthetic construct
  source 1..29: mol_type = other DNA
accctaaccctaaccctcacccttctaac

SEQ ID NO: 486 - DNA, 29 bp, synthetic construct
  source 1..29: mol_type = other DNA
accctcaccctaaccctaaccctcaccct

SEQ ID NO: 487 - DNA, 29 bp, synthetic construct
  source 1..29: mol_type = other DNA
accctagcactaaccctaaccctgatctc

SEQ ID NO: 488 - DNA, 29 bp, synthetic construct
  source 1..29: mol_type = other DNA
ctatcaccaccatcatccaactcaggaaa

SEQ ID NO: 489 - DNA, 29 bp, synthetic construct
  source 1..29: mol_type = other DNA
gttgctgcagtaaatcctaactgtaaact

SEQ ID NO: 490 - DNA, 29 bp, synthetic construct
  source 1..29: mol_type = other DNA
tctacagatttctcccctcactcagttct

SEQ ID NO: 491 - DNA, 29 bp, synthetic construct
  source 1..29: mol_type = other DNA
cagcccgctgttattttcacctgtagtgt

SEQ ID NO: 492 - DNA, 29 bp, synthetic construct
  source 1..29: mol_type = other DNA
ggctcagtcctaaagactgcctccaattc

SEQ ID NO: 493 - DNA, 23 bp, synthetic construct
  source 1..23: mol_type = other DNA
tccgctttaacccccacctcnnn

SEQ ID NO: 494 - DNA, 23 bp, synthetic construct
  source 1..23: mol_type = other DNA
tccgctttaacccccacctccag

SEQ ID NO: 495 - DNA, 23 bp, synthetic construct
  source 1..23: mol_type = other DNA
ctggcttcaacccccaccactgc

SEQ ID NO: 496 - DNA, 23 bp, synthetic construct
  source 1..23: mol_type = other DNA
tctgctttaacccccacctccta

SEQ ID NO: 497 - DNA, 23 bp, synthetic construct
  source 1..23: mol_type = other DNA
cccacctcaacccccacctctga

SEQ ID NO: 498 - DNA, 23 bp, synthetic construct
  source 1..23: mol_type = other DNA
ctccctgtaacccccacctcaaa

SEQ ID NO: 499 - DNA, 23 bp, synthetic construct
  source 1..23: mol_type = other DNA
tgagctttaacccccacctcatt

SEQ ID NO: 500 - DNA, 23 bp, synthetic construct
  source 1..23: mol_type = other DNA
gcagccttaacccccacctcagc

SEQ ID NO: 501 - DNA, 23 bp, synthetic construct
  source 1..23: mol_type = other DNA
tccgccttgacccccacctccac

SEQ ID NO: 502 - DNA, 23 bp, synthetic construct
  source 1..23: mol_type = other DNA
tccacaaaaacccccacctctga

SEQ ID NO: 503 - DNA, 23 bp, synthetic construct
  source 1..23: mol_type = other DNA
cccgcctcaacccccacctctgt

SEQ ID NO: 504 - DNA, 23 bp, synthetic construct
  source 1..23: mol_type = other DNA
gctgcttcaacccccacctccca

SEQ ID NO: 505 - DNA, 23 bp, synthetic construct
  source 1..23: mol_type = other DNA
gtccgctccacccccacctcctg

SEQ ID NO: 506 - DNA, 23 bp, synthetic construct
  source 1..23: mol_type = other DNA
ctcgcttcaacccccacctcttg

SEQ ID NO: 507 - DNA, 23 bp, synthetic construct
  source 1..23: mol_type = other DNA
tccgcttcaccccccacctccag

SEQ ID NO: 508 - DNA, 23 bp, synthetic construct
  source 1..23: mol_type = other DNA
tcagccttaacccccacctcaat

SEQ ID NO: 509 - DNA, 23 bp, synthetic construct
  source 1..23: mol_type = other DNA
tccactccaacccccacctcgct

SEQ ID NO: 510 - DNA, 23 bp, synthetic construct
  source 1..23: mol_type = other DNA
cccactctaacccccactccacc

SEQ ID NO: 511 - DNA, 23 bp, synthetic construct
  source 1..23: mol_type = other DNA
ctggcttcaacccccacctcacc

SEQ ID NO: 512 - DNA, 23 bp, synthetic construct
  source 1..23: mol_type = other DNA
acatccacaacccccacctcagc

SEQ ID NO: 513 - DNA, 23 bp, synthetic construct
  source 1..23: mol_type = other DNA
cggctttcaacccccacctcagc

SEQ ID NO: 514 - DNA, 23 bp, synthetic construct
  source 1..23: mol_type = other DNA
gtggcttcaacccccaccacagt

SEQ ID NO: 515 - DNA, 23 bp, synthetic construct
  source 1..23: mol_type = other DNA
ctcactgcaacctccacttcctg

SEQ ID NO: 516 - DNA, 23 bp, synthetic construct
  source 1..23: mol_type = other DNA
gatgccttaacccccaccccagg

SEQ ID NO: 517 - DNA, 23 bp, synthetic construct
  source 1..23: mol_type = other DNA
tttgccttaacccccacctctac

SEQ ID NO: 518 - DNA, 23 bp, synthetic construct
  source 1..23: mol_type = other DNA
atcactttaacctccacctctgg

SEQ ID NO: 519 - DNA, 23 bp, synthetic construct
  source 1..23: mol_type = other DNA
cccgcatggacccccacctccac

SEQ ID NO: 520 - DNA, 23 bp, synthetic construct
  source 1..23: mol_type = other DNA
gcccccccacccccccccccccg

SEQ ID NO: 521 - DNA, 23 bp, synthetic construct
  source 1..23: mol_type = other DNA
ctaaccctaaccccaaccccaaa

SEQ ID NO: 522 - DNA, 23 bp, synthetic construct
  source 1..23: mol_type = other DNA
ctagctttaacccccatctccac

SEQ ID NO: 523 - DNA, 23 bp, synthetic construct
  source 1..23: mol_type = other DNA
atccctttatcactcacctaaca

SEQ ID NO: 524 - DNA, 23 bp, synthetic construct
  source 1..23: mol_type = other DNA
catacttcaacccccacctcttg

SEQ ID NO: 525 - DNA, 23 bp, synthetic construct
  source 1..23: mol_type = other DNA
gtccactcaactcccacctctac

SEQ ID NO: 526 - DNA, 23 bp, synthetic construct
  source 1..23: mol_type = other DNA
tatcccctaacccccacctccaa

SEQ ID NO: 527 - DNA, 23 bp, synthetic construct
  source 1..23: mol_type = other DNA
ctcgcctcaacccccacctccca

SEQ ID NO: 528 - DNA, 23 bp, synthetic construct
  source 1..23: mol_type = other DNA
tctgcttcagggcccgctgcact

SEQ ID NO: 529 - DNA, 23 bp, synthetic construct
  source 1..23: mol_type = other DNA
tccactttaacccccacaccgga

SEQ ID NO: 530 - DNA, 23 bp, synthetic construct
  source 1..23: mol_type = other DNA
gcagtcctaacccccacctccac

SEQ ID NO: 531 - DNA, 23 bp, synthetic construct
  source 1..23: mol_type = other DNA
ctcctaagaacccccacctccaa

SEQ ID NO: 532 - DNA, 23 bp, synthetic construct
  source 1..23: mol_type = other DNA
gcggctgaaatctccacttgcaa

SEQ ID NO: 533 - DNA, 23 bp, synthetic construct
  source 1..23: mol_type = other DNA
acacgctgagcccccaccaccag

SEQ ID NO: 534 - DNA, 23 bp, synthetic construct
  source 1..23: mol_type = other DNA
aggagtgaaacccccacctctaa

SEQ ID NO: 535 - DNA, 23 bp, synthetic construct
  source 1..23: mol_type = other DNA
cccactggatacctaacctctgt

SEQ ID NO: 536 - DNA, 23 bp, synthetic construct
  source 1..23: mol_type = other DNA
tcggttgatccacccacctgggc

SEQ ID NO: 537 - DNA, 23 bp, synthetic construct
  source 1..23: mol_type = other DNA
tcatccttggctcccatctcccc

SEQ ID NO: 538 - DNA, 23 bp, synthetic construct
  source 1..23: mol_type = other DNA
tctctggtgagccccacctgacc

SEQ ID NO: 539 - DNA, 23 bp, synthetic construct
  source 1..23: mol_type = other DNA
taacccttaacccctaaccccta

SEQ ID NO: 540 - DNA, 23 bp, synthetic construct
  source 1..23: mol_type = other DNA
accaccctaaccctaaccctaac

SEQ ID NO: 541 - DNA, 23 bp, synthetic construct
  source 1..23: mol_type = other DNA
tcagctctgcctcccgctacgtt

SEQ ID NO: 542 - DNA, 29 bp, synthetic construct
  source 1..29: mol_type = other DNA
ccctcatctccaatatggtatggnnnnnn

SEQ ID NO: 543 - DNA, 29 bp, synthetic construct
  source 1..29: mol_type = other DNA
ccctcatctccaatatggtatggcggccc

SEQ ID NO: 544 - DNA, 29 bp, synthetic construct
  source 1..29: mol_type = other DNA
ccagcctgggcaacatagcaagaccctat

SEQ ID NO: 545 - DNA, 28 bp, synthetic construct
  source 1..28: mol_type = other DNA
cactcctcagcaaatgcaaaagaataga

SEQ ID NO: 546 - DNA, 29 bp, synthetic construct
  source 1..29: mol_type = other DNA
cactcagcccaacttagaaatttttaaaa

SEQ ID NO: 547 - DNA, 29 bp, synthetic construct
  source 1..29: mol_type = other DNA
tgccgatctcatattagtattggtttctt

SEQ ID NO: 548 - DNA, 29 bp, synthetic construct
  source 1..29: mol_type = other DNA
gttttgtgataaatatggttaggttaaaa

SEQ ID NO: 549 - DNA, 29 bp, synthetic construct
  source 1..29: mol_type = other DNA
atttcatctcagaagtcttagtgatttga

SEQ ID NO: 550 - DNA, 29 bp, synthetic construct
  source 1..29: mol_type = other DNA
cacacatcacaaaggagtttctgagaatc

SEQ ID NO: 551 - DNA, 29 bp, synthetic construct
  source 1..29: mol_type = other DNA
cccttctttttctttttctttggtttttt

SEQ ID NO: 552 - DNA, 29 bp, synthetic construct
  source 1..29: mol_type = other DNA
ctatctgagccaatgtgggtgtggctggg

SEQ ID NO: 553 - DNA, 29 bp, synthetic construct
  source 1..29: mol_type = other DNA
ccgtgttggccaggatggtctccatctct

SEQ ID NO: 554 - DNA, 29 bp, synthetic construct
  source 1..29: mol_type = other DNA
aactgatcttcagcaaagtaaacaaaaac

SEQ ID NO: 555 - DNA, 29 bp, synthetic construct
  source 1..29: mol_type = other DNA
ccctcagcactttaaatatatcattacca

SEQ ID NO: 556 - DNA, 29 bp, synthetic construct
  source 1..29: mol_type = other DNA
cccttctctataatatgaaggtatctttt

SEQ ID NO: 557 - DNA, 29 bp, synthetic construct
  source 1..29: mol_type = other DNA
aggtgatatctaattgtgttttgtatttg

SEQ ID NO: 558 - DNA, 29 bp, synthetic construct
  source 1..29: mol_type = other DNA
tacacatgtgcaatatacattttatgcat

SEQ ID NO: 559 - DNA, 29 bp, synthetic construct
  source 1..29: mol_type = other DNA
tcctcatgaatgatttggtgccctcccca

SEQ ID NO: 560 - DNA, 29 bp, synthetic construct
  source 1..29: mol_type = other DNA
tccacaccccaagcctgctattgaaagag

SEQ ID NO: 561 - DNA, 29 bp, synthetic construct
  source 1..29: mol_type = other DNA
ccctaacccgggttaatgtgaggtgcaag

SEQ ID NO: 562 - DNA, 29 bp, synthetic construct
  source 1..29: mol_type = other DNA
ctcttagcaccacttttgttgtgtcccag

SEQ ID NO: 563 - DNA, 29 bp, synthetic construct
  source 1..29: mol_type = other DNA
cccccccccccaaaacaaaaaagccaaaa

SEQ ID NO: 564 - DNA, 29 bp, synthetic construct
  source 1..29: mol_type = other DNA
ccctcccccccaacgtagctgggattaca

SEQ ID NO: 565 - DNA, 29 bp, synthetic construct
  source 1..29: mol_type = other DNA
ccatcctctgctgcatttattggggtgga

SEQ ID NO: 566 - DNA, 29 bp, synthetic construct
  source 1..29: mol_type = other DNA
ccagtttttcaaagatgccttgcctccta

SEQ ID NO: 567 - DNA, 29 bp, synthetic construct
  source 1..29: mol_type = other DNA
ttcttctgtctagcatgaaatgaagaaat

SEQ ID NO: 568 - DNA, 29 bp, synthetic construct
  source 1..29: mol_type = other DNA
atatagactctaatataatttgacaaaaa

SEQ ID NO: 569 - DNA, 29 bp, synthetic construct
  source 1..29: mol_type = other DNA
ttctcagaacaaaccaggtaagtggtctt

SEQ ID NO: 570 - DNA, 29 bp, synthetic construct
  source 1..29: mol_type = other DNA
aattcagttcaattactgtatatggcggg

SEQ ID NO: 571 - DNA, 29 bp, synthetic construct
  source 1..29: mol_type = other DNA
cctttaatactgtttttgtatggtgctgc

SEQ ID NO: 572 - DNA, 29 bp, synthetic construct
  source 1..29: mol_type = other DNA
ccatccttccttatggcctatgcattctt

SEQ ID NO: 573 - DNA, 29 bp, synthetic construct
  source 1..29: mol_type = other DNA
ttgtagtttccaatatactgttaagccca

SEQ ID NO: 574 - DNA, 29 bp, synthetic construct
  source 1..29: mol_type = other DNA
cccttgcctccaactaagaagccttacaa

SEQ ID NO: 575 - DNA, 29 bp, synthetic construct
  source 1..29: mol_type = other DNA
gcattctttggaaaagggaatgtcttcac

SEQ ID NO: 576 - DNA, 23 bp, synthetic construct
  source 1..23: mol_type = other DNA
tcatctccaatatggtatggnnn

SEQ ID NO: 577 - DNA, 23 bp, synthetic construct
  source 1..23: mol_type = other DNA
tcatctccaatatggtatggcgg

SEQ ID NO: 578 - DNA, 23 bp, synthetic construct
  source 1..23: mol_type = other DNA
tcatccccaatatggtgggcatc

SEQ ID NO: 579 - DNA, 23 bp, synthetic construct
  source 1..23: mol_type = other DNA
tattctacaatatggtatatgga

SEQ ID NO: 580 - DNA, 23 bp, synthetic construct
  source 1..23: mol_type = other DNA
tcatctccaataatggtaagagt

SEQ ID NO: 581 - DNA, 23 bp, synthetic construct
  source 1..23: mol_type = other DNA
tatcatccaataaggtatatggt

SEQ ID NO: 582 - DNA, 23 bp, synthetic construct
  source 1..23: mol_type = other DNA
tcatcagaaaaatggaaatcaaa

SEQ ID NO: 583 - DNA, 23 bp, synthetic construct
  source 1..23: mol_type = other DNA
ctatctccaatatgttatgggga

SEQ ID NO: 584 - DNA, 23 bp, synthetic construct
  source 1..23: mol_type = other DNA
ttatatacaatagtgttggaagt

SEQ ID NO: 585 - DNA, 23 bp, synthetic construct
  source 1..23: mol_type = other DNA
ccctcattgatatggtttggctg

SEQ ID NO: 586 - DNA, 23 bp, synthetic construct
  source 1..23: mol_type = other DNA
ccatctccaataatggagactgc

SEQ ID NO: 587 - DNA, 23 bp, synthetic construct
  source 1..23: mol_type = other DNA
tcagctctactttcctatgctag

SEQ ID NO: 588 - DNA, 23 bp, synthetic construct
  source 1..23: mol_type = other DNA
gcacctccaatatggtggcagga

SEQ ID NO: 589 - DNA, 23 bp, synthetic construct
  source 1..23: mol_type = other DNA
tcatttccaatatggtaggtgtc

SEQ ID NO: 590 - DNA, 23 bp, synthetic construct
  source 1..23: mol_type = other DNA
ttatctccaatatggtatagagt

SEQ ID NO: 591 - DNA, 23 bp, synthetic construct
  source 1..23: mol_type = other DNA
tcacctccaatattatagagacc

SEQ ID NO: 592 - DNA, 23 bp, synthetic construct
  source 1..23: mol_type = other DNA
ccatctcaaaaaaagaaaagaaa

SEQ ID NO: 593 - DNA, 23 bp, synthetic construct
  source 1..23: mol_type = other DNA
acatatgcactacggcatggaac

SEQ ID NO: 594 - DNA, 23 bp, synthetic construct
  source 1..23: mol_type = other DNA
atatctagcacatggtaaagaca

SEQ ID NO: 595 - DNA, 80 bp, synthetic construct
  source 1..80: mol_type = other DNA
aaaaggtactgttactgtatattcatctgacgttaaacctgaaaatctacgcaatttctttatttctgttttacgtgcaa

SEQ ID NO: 596 - DNA, 78 bp, synthetic construct
  source 1..78: mol_type = other DNA
ttacttggcgcgactaaaaaattgaatgtaggagctgccgctattgttcttcccacagcatttctgttttacgtgcaa

SEQ ID NO: 597 - DNA, 78 bp, synthetic construct
  source 1..78: mol_type = other DNA
ttacttggcgcgactaaaaaattgaatgtaggaactgccgctattgttcttcccacagcatttctgttttacgtgcaa

SEQ ID NO: 598 - DNA, 32 bp, synthetic construct
  source 1..32: mol_type = other DNA
ggcagacttctccacaggagtcaggtgcacca

SEQ ID NO: 599 - DNA, 60 bp, synthetic construct
  source 1..60: mol_type = other DNA
ctgacgttaaacctgaaaatctacgcaatttctttatttctgttttacgtgcaaataatt

SEQ ID NO: 600 - protein, 19 aa, synthetic construct
DVKPENLRNFFISVLRANN

SEQ ID NO: 601 - DNA, 51 bp, synthetic construct
  source 1..51: mol_type = other DNA
aaaaaattattattcgcaattcctttatgtctcagctacgaaactgaaatc

SEQ ID NO: 602 - protein, 17 aa, synthetic construct
KKLLFAIPLCLSYETEI

SEQ ID NO: 603 - protein, 5 aa, Neisseria meningitidis
HQKTH

SEQ ID NO: 604 - protein, 5 aa, synthetic construct
HRRTH

SEQ ID NO: 605 - protein, 5 aa, synthetic construct
HQRTH

SEQ ID NO: 606 - protein, 5 aa, synthetic construct
HQRTR

SEQ ID NO: 607 - protein, 5 aa, synthetic construct
HQKAH

SEQ ID NO: 608 - protein, 5 aa, synthetic construct
RQKAH

SEQ ID NO: 609 - protein, 5 aa, synthetic construct
YRKTL

SEQ ID NO: 610 - protein, 5 aa, synthetic construct
HRKTH

SEQ ID NO: 611 - DNA, 29 bp, synthetic construct
  source 1..29: mol_type = other DNA
tgttcctcactactcagagccagnnnnyn

SEQ ID NO: 612 - DNA, 34 bp, synthetic construct
  source 1..34: mol_type = other DNA
ccaccctcatctccaatatggtatggcggccctt

SEQ ID NO: 613 - DNA, 23 bp, synthetic construct
  source 1..23: mol_type = other DNA
ccaccctcatctccaatatggta

SEQ ID NO: 614 - DNA, 11 bp, synthetic construct
  source 1..11: mol_type = other DNA
tggcggccctt

SEQ ID NO: 615 - DNA, 29 bp, synthetic construct
  source 1..29: mol_type = other DNA
tgttccgccctactcagagccagaggtcc

SEQ ID NO: 616 - protein, 8 aa, synthetic construct
XSGSEGGT

SEQ ID NO: 617 - DNA, 29 bp, synthetic construct
  source 1..29: mol_type = other DNA
tgttccgccctactcagagccagaggccc

SEQ ID NO: 618 - DNA, 29 bp, synthetic construct
  source 1..29: mol_type = other DNA
tgttcctcactactcagagccagtccnnn

SEQ ID NO: 619 - protein, 7 aa, synthetic construct
XSGSEGT

SEQ ID NO: 620 - DNA, 29 bp, synthetic construct
  source 1..29: mol_type = other DNA
tgttcctcgctgctcagagccagtccnnn

SEQ ID NO: 621 - protein, 9 aa, synthetic construct
XSGSERRGT

SEQ ID NO: 622 - DNA, 32 bp, synthetic construct
  source 1..32: mol_type = other DNA
gcaatggccgggactcctgggagcactctccc

(19) United States
(12) Patent Application Publication
(10) Pub. No.: US 2025/0027114 A1
(43) Pub. Date: Jan. 23, 2025

(54) CAS9 VARIANTS HAVING NON-CANONICAL PAM SPECIFICITIES AND USES THEREOF

(71) Applicants: The Broad Institute, Inc., Cambridge, MA (US); President and Fellows of Harvard College, Cambridge, MA (US); Trustees of Boston University, Boston, MA (US)

(72) Inventors: David R. Liu, Cambridge, MA (US); Tony P. Huang, Cambridge, MA (US); Zachary J. Heins, Boston, MA (US); Ahmad S. Khalil, Boston, MA (US)

(73) Assignees: The Broad Institute, Inc., Cambridge, MA (US); President and Fellows of Harvard College, Cambridge, MA (US); Trustees of Boston University, Boston, MA (US)

(21) Appl. No.: 18/906,028

(22) Filed: Oct. 3, 2024

Related U.S. Application Data
(63) Continuation of application No. PCT/US2023/065312, filed on Apr. 4, 2023.
(60) Provisional application No. 63/396,943, filed on Aug. 10, 2022.
(60) Provisional application No. 63/327,354, filed on Apr. 4, 2022.

(51) Int. Cl. C12N 15/90; C12N 9/22; C12N 9/78; C12N 15/11

(52) U.S. Cl. CPC C12N 15/90; C12N 9/22; C12N 9/78; C12N 15/11; C12N 2310/20

(57) ABSTRACT

The present disclosure provides Cas9 variants, and base editors comprising these variants, that recognize non-canonical protospacer adjacent motifs (PAMs) and have less restrictive PAM requirements for editing. The present disclosure provides Cas9 protein variants comprising one or more amino acid substitutions relative to wild-type Nme2Cas9. Fusion proteins comprising the Cas protein variants described herein are also provided by the present disclosure. Further provided herein are methods for editing a target nucleic acid using the Cas variants and fusion proteins provided herein. The present disclosure also provides guide RNAs, complexes, polynucleotides, cells, kits, and pharmaceutical compositions. Further described herein are phage-assisted continuous evolution (PACE) systems, vectors, methods, and devices.

### RELATED APPLICATIONS

This application claims the benefit under 35 U.S.C. § 119(e) to U.S. Provisional Application No. 63/327,354 entitled “CAS9 VARIANTS HAVING NON-CANONICAL PAM SPECIFICITIES AND USES THEREOF”, filed on Apr. 4, 2022, and U.S. Provisional Application No. 63/396,943 entitled “CAS9 VARIANTS HAVING NON-CANONICAL PAM SPECIFICITIES AND USES THEREOF”, filed on Aug. 10, 2022; the contents of each of which are incorporated herein by reference in their entirety.

### GOVERNMENT SUPPORT

This invention was made with government support under Grant Nos. EB027793, EB031172, A1142756, GM118062, HG009490 and GM119228 awarded by the National Institutes of Health; Grant Nos. CCF-202045 and EF-1921677 awarded by the National Science Foundation, and Grant No. N0014-20-1-2825 awarded by the Department of Defense. The government has certain rights in the invention.

### REFERENCE TO AN ELECTRONIC SEQUENCE LISTING

The contents of the electronic sequence listing (B119570161WO00-SEQ-VLJ.xml; Size: 613,123 bytes; and Date of Creation: Apr. 3, 2023) is herein incorporated by reference in its entirety.

### BACKGROUND OF THE INVENTION

CRISPR-Cas systems have successfully been engineered for genome editing and base editing in a wide range of organisms. Naturally occurring and laboratory-created Cas9 variants have provided a suite of Cas9 proteins that engage DNA targets at a variety of protospacer-adjacent motif (PAM) sequences.

One drawback of current genomic and base editing tools is that the PAM requirements of existing Cas9 proteins limits the applicability of precision gene editing methods, as the target modification must occur either at a specific distance or within a certain range of the PAM sequence. PAM recognition by the Cas domain of a base editor precedes a) formation of an R loop in the target nucleic acid molecule, and b) the interaction between the Cas9:guide RNA complex and the target molecule. The availability of a PAM sequence compatible with a Cas domain that retains robust activity in mammalian cells strongly determines the scope of base editing. In this respect, the PAM serves as a gatekeeper for Cas9 binding and recognition. In particular, the PAM requirement can limit the scope of base editing applications because it restricts the recognition of many homologs of Cas9 for various target sites of clinical interest, such as single-nucleotide polymorphisms (SNPs). Conversely, the scope of editing activity should not be so wide as to generate off-target effects.

Accordingly, there is a need for novel engineered Cas9 proteins with broad PAM sequence compatibility that enable efficient base editing and generate few off-target effects.

### SUMMARY OF THE INVENTION

The present disclosure provides Cas9 variants, and base editors comprising these variants, that have less restrictive PAM requirements for editing. These Cas9 variants recognize PAMs that are not recognized by existing Cas9 variants and orthologs. Accordingly, Cas9 variants and base editors having non-canonical PAM specificities are provided. In particular, Cas9 variants and base editors having specificity for PAMs containing a single pyrimidine (cytosine (C) and thymine (T)) are described. These Cas9 variants have non-canonical PAM specificities that enable targeting of most pyrimidine-rich PAM sequences, substantially expanding the capabilities of Cas9 to edit desired therapeutic targets that may be poorly accessed by existing Cas proteins. The disclosed Cas variants may also have expanded capabilities for nucleic acid cleavage, prime editing, and recombination; as well as expanded uses as transcription factors and epigenetic modifiers. Base editors comprising any of the disclosed Cas9 variants are provided.

The present disclosure also provides complexes of disclosed base editors and a guide RNA. The present disclosure further provides polynucleotides and vectors encoding the disclosed Cas9 variants and base editors, and kits and compositions containing these vectors. The present disclosure also provides methods of editing a target nucleic acid sequence with any of the disclosed base editors.

The present disclosure further provides new systems of continuous and non-continuous evolution of proteins, such as non-canonical Cas9 homologs. Accordingly, in some aspects, novel methods, vectors, and cells are provided related to sequence-agnostic Cas evolution selection systems, which are herein referred to as “SAC-PACE”. In some aspects, SAC-PACE evolves proteins (e.g., Cas9) based on degree of PAM recognition and binding, as well as base editing. In some embodiments, SAC-PACE may involve a negative selection aspect. In some embodiments, SAC-PACE may be used in combination with the eVOLVER system (referred to as ePACE) to perform parallel PACE selections, including massively parallel selections. In some embodiments, the eVOLVER system contains millifluidic devices that allow for efficient and cost-effective scale-up of protein evolution. Some aspects of the present disclosure relate to compositions of Nme2Cas protein variants evolved using an ePACE strategy. In some embodiments, the Nme2Cas protein variants are evaluated for their ability to edit genomic sequences using a novel profiling system referred to herein as the base editing dependent PAM profiling assay (BE-PPA).

Accordingly, described herein are improved phage-assisted continuous evolution (PACE) systems, vectors, methods, and devices. In particular, described herein are massively parallel PACE systems that utilize peristaltic systems of millifluidic volume. In particular, provided herein are systems and devices that facilitate scaling of complex fluidic manipulations for which individual control of multiple fluidic routes is desired without the need for multiple external mechanical pumps. These systems and devices may be applied to any fluidic operation where control of complex fluidic routines-such as mixing, pumping, isolating, merging, and storing—is desired at larger scales than microfluidic volumes can provide. These systems contain improvements upon earlier PACE systems, such as those described in US Patent Publication No. 2021/0214713, and Heins et al., J Vis Erp. 2019 May (147), e59652, each of which is incorporated by reference herein. The development of ePACE facilitated parallel, automated, and fully continuous evolution of Nme2Cas9 on multiple PAMs, overcoming many of the design, operation, and infrastructural challenges of traditional PACE.

Described herein is the SAC-PACE evolution system, which increases the likelihood of evolving desired editing properties, such as the evolution of any Cas ortholog towards recognition of novel PAMs. The sequence-agnostic nature of the target site may be applied to evolving novel editing windows or disease-specific contexts. SAC-PACE is a directed system for evolution of any Cas ortholog to evolve altered PAM compatibility based on a combination of elements of a DNA-binding selection with a base editing (BE) selection. Accordingly, SAC-PACE may be used for evolution of Cas variants derived from any bacterial system, such as C. jejuni, N. meningitis, S. aureus, and S. pyogenes. SAC-PACE may be used for the evolution of Cas homologs other than Cas9, such as Cas12, Cas14, Cas14a1, Cpf1, CasX, CasY, C2c1, C2c2, C2c3, SpRY, CjCas9, and Argonaute (Ago) homologs. This disclosure is based in part on the discovery that functional selection enables improved evolution outcomes, in particular for Cas variants with lower starting activity. This selection system is broadly adaptable to the evolution of any Cas ortholog towards novel PAMs, and the sequence-agnostic nature of the target site can be applied to evolving novel editing windows or disease-specific contexts. SAC-PACE differs from existing PACE systems for base editing in that it is dependent on R-loop formation/activation as well as PAM binding, rather than solely PAM binding or solely editing activity. In particular, SAC-PACE requires both novel PAM binding and subsequent activation steps necessary for base editing, increasing the likelihood of evolving desired editing properties This system enables higher stringencies than existing PACE systems for PAM compatibility selection. For instance, the dual-PAM SAC-PACE systems described herein are adapted for very high stringency of selection. SAC-PACE further enables direct multiplexing (i.e., multiple PAM/protospacers), and is resistant to introduction of bystander edits during selection.

The present disclosure describes the use of multiple rounds of phage-assisted non-continuous evolution (PANCE) and eVOLVER-supported phage-assisted continuous evolution (ePACE) of a compact Cas9 ortholog from Neisseria meningitidis—Nme2Cas9—to yield several variants with improved activity (e.g., base editing activity when fused to a deaminase) in host cells. The advantages of Nme2Cas9 are that it is active on a simple dinucleotide PAM, has a smaller size relative to SpCas9, and has shown robust activity in mammalian cells as a nuclease and in a base editor. Wild-type Nme2Cas9 (N. meningitidis isoform 2 Cas9) recognizes the PAM NNNNCC, or N₄CC (where N is any nucleotide) (or more simply, “CC”). The variants of Nme2Cas9 provided herein recognize a wider array of PAMs than N₄CC. In particular, the disclosed variants recognize PAMs of the sequence NYN, where Y is any pyrimidine (i.e., C, T, or U). For instance, the disclosed variants recognize PAMs of the sequence NCN (or “C”) and NTN (or “T”). Thus, the disclosed Cas9 variants recognize single-nucleotide-pyrimidine PAMs.

The disclosed Cas9 variants enable targeting of most pyrimidine-rich PAM sequences, including those poorly accessed by existing Cas proteins. Thus, the disclosed variants substantially expand the targeting capabilities of Cas9-based technologies, such as base editing and epigenetic modification, to areas of the genome with different PAM requirements.

CRISPR-Cas9 has enabled the development of genome-manipulating technologies that have transformed the life sciences and advanced new treatments for genetic disorders into the clinic. Target sites engaged by Cas9 must contain a protospacer adjacent motif (PAM) that is recognized through a protein:DNA interaction prior to single guide RNA (sgRNA) binding. While not prohibitive for some gene editing applications, such as target gene disruption, this PAM requirement limits the applicability of precision gene editing methods, including base editing, prime editing, or site-specific DNA integration. For these technologies, the target modification must occur either at a specific distance or within a certain range of the PAM. Thus, the availability of a PAM sequence compatible with a Cas protein that retains robust activity in mammalian cells strongly determines the application scope of precision gene editing. Indeed, recent ex vivo and in vivo therapeutic base editing to rescue sickle-cell disease and progeria in mice used evolved or engineered Cas9 variants to precisely position the base editor at CACC or NGA PAMs, respectively. See International Patent Publication Nos. WO 2020/051360, published Mar. 12, 2020, and WO 2019/079347, published Apr. 25, 2019, each of which is herein incorporated by reference.

The genomes of other bacterial species or bacteriophages have also been parsed to identify Cas variants with different PAM requirements. These Cas variants vary dramatically in size, PAM compatibility, and enzymatic activity. However, most of these natural homologs are less well characterized, less active in mammalian cells, or have highly restrictive PAM requirements compared to SpCas9, limiting their utility for precision gene editing applications and the ease with which they can be modified. As such, engineering or evolution of non-SpCas9 orthologs has been uncommon, with only a few reported examples.

Novel engineering or evolution methods to address the limitations of reprogramming non-SpCas9 orthologs could provide new precision gene editing capabilities that expand upon and complement the suite of commonly used SpCas9-derived variants. Nme2Cas9 is an attractive Cas ortholog for evolving PAM compatibility. The wild-type enzyme is active on N₄CC PAMs, and thus may serve as a promising starting point to all pyrimidine PAMs previously inaccessible by SpCas9 variants. Nme2Cas9 has also shown robust activity in mammalian cells as both a nuclease and a base editor. It is also a compact Cas ortholog, having a length of 1082 residues.

Accordingly, in one aspect, the present disclosure provides Cas variants comprising an amino acid sequence that is at least 70%, at least 75%, at least 80%, at least 85%, at least 90%, at least 95%, or at least 99% identical to the amino acid sequence of a wild-type Nme2Cas9 protein of SEQ ID NO: 5. In some embodiments, the amino acid sequence of the Cas variant comprises at least 1, at least 2, at least 3, at least 4, at least 5, at least 6, at least 7, at least 8, at least 9, at least 10, at least 11, at least 12, at least 15, at least 20, or at least 25 substitutions at positions selected from the group consisting of amino acid residues 6, 33, 47, 63, 68, 104, 116, 123, 152, 154, 221, 260, 263, 303, 396, 413, 427, 451, 452, 460, 484, 520, 629, 646, 674, 696, 711, 720, 724, 758, 765, 767, 769, 771, 816, 821, 844, 859, 865, 932, 940, 951, 1005, 1028, 1029, 1031, 1033, 1044, 1047, 1049, 1056, 1064, and 1075 of the amino acid sequence provided in SEQ ID NO: 5, or corresponding positions in other Cas homologs (i.e., a protein related to another Cas protein through a common ancestral sequence). In some embodiments, the Cas variants comprise substitutions selected from the group consisting of P6X, E33X, E47X, R63X, V68X, K104X, A116X, T123X, D152X, E154X, E221X, F260X, A263X, A303X, T396X, H413X, A427X, D451X, H452X, E460X, A484X, E520X, S629X, R646X, N674X, F696X, G711X. D720X, A724X, 1758X, V765X, H767X, K769X, H771X, S816X, V821X, D844X, 1859X, W865X, E932X, K940X, M951X, K1005X, D1028X, S1029X, N1031X, R1033X, K1044X, Q1047X, R1049X, V1056X, N1064X, and L1075X, relative to the amino acid sequence provided in SEQ ID NO: 5, wherein X represents any amino acid, or corresponding mutations in other Cas homologs. In certain embodiments, the Cas variants comprise substitutions selected from the group consisting of P6S, E33G, E47K, R63K, V68M, K104T, A116T, T123A, D152A, D152N, D152G, E154K, E221D, F260L, A263T, A303S, T396A, H413N, A427S, D451V, H452R, E460A, E460K, A484T, E520A, S629P, R646S, N674S, F696V, G711R, D720A, A724S, 1758V, V765A, H767Y, K769R, H771R, S8161, V821A, D844A, 1859V, W865L, E932K, K940R, M951R, K1005R, D1028N, S1029A, N1031S, R1033N, R1033G, R1033Y, K1044R, R1049S, R1049C, Q1047R, V1056A, N1064S, and L1075M, relative to the amino acid sequence of SEQ ID NO: 5, or corresponding mutations in other Cas homologs.

In some embodiments, the Cas variants comprise at least 5, at least 6, at least 7, at least 8, at least 9, at least 10, at least 11, at least 12, at least 15, at least 20, at least 25, at least 26, at least 27, at least 28, or more than 28 substitutions at positions selected from the group consisting of amino acid residues 6, 33, 47, 63, 68, 104, 116, 123, 152, 154, 221, 260, 263, 303, 396, 413, 427, 451, 452, 460, 484, 520, 629, 646, 674, 696, 711, 720, 724, 758, 765, 767, 769, 771, 816, 821, 844, 859, 865, 932, 940, 951, 1005, 1028, 1029, 1031, 1033, 1044, 1047, 1049, 1056, 1064, and 1075 of the amino acid sequence provided in SEQ ID NO: 5.

The disclosed Cas variants show preference for novel PAM sequences. In some embodiments, the Cas variant is eNme2-C(SEQ ID NO: 1). In some embodiments, the Cas variant is eNme2-C.NR (SEQ ID NO: 4). The eNme2-C and eNme2-C.NR variants show a preference for targeting NNNNCN PAMs. In some embodiments, the Cas variant is eNme2-T.1 (SEQ ID NO: 2). In some embodiments, the Cas variant is eNme2-T.2 (SEQ ID NO: 3). The eNme2-T.1 and eNme2-T.2 variants show a preference for targeting NNNNTN PAMs. In some embodiments, the Cas variant is eNme2-N1-21. The eNme2-N1-21 variant shows a preference for targeting NNNTTG PAMs. In various embodiments, base editors containing any of the disclosed eNme2Cas9 variants are compatible with more PAMs and/or have higher specificity of binding to more PAMs than base editors containing the SpRY variant.

In another aspect, the present disclosure provides fusion proteins. In some embodiments, the fusion proteins comprise (i) a Cas protein variant provided herein; and (ii) an effector domain. In certain embodiments, the effector domain is a nucleic acid editing domain, such as a deaminase domain (i.e., the fusion protein is a base editor). In some embodiments, the base editors of the disclosure have low off-target editing activity, e.g., as measured by CRISPResso. In some embodiments, the disclosed base editors contain a Cas domain that comprises the eNme2-C variant. Base editors containing the eNme2-C variant generated efficiencies of base editing of about 60% or higher on N₄CC PAMs in human cells, as disclosed in Example 1, which represents a two-fold improvement relative to base editors containing wild-type Nme2Cas9. In some embodiments, the disclosed base editors contain a Cas domain that comprises the eNme2-C.NR variant. In some embodiments, the base editor is an adenine base editor (ABE). In some embodiments, the base editor is a cytosine base editor. In some embodiments, the base editor comprises the structure: NH2-[adenosine deaminase]-[Cas9 protein]-COOH; or NH2-[Cas9 protein]-[adenosine deaminase]-COOH, wherein each “]-[” in the structure indicates the presence of an optional linker sequence.

The disclosed base editors provide options for base editing and maintenance of low indel formation on cytosine-containing PAMs. The disclosed base editors further enable access to additional target sites in the genome where therapeutically relevant point mutations or reversions may be made. The disclosed base editors generate high editing efficiencies in multiple human cell types, such as HUH7, U20S, and HDFa cell lines.

In another aspect, the present disclosure provides guide RNAs (gRNAs) comprising a guide sequence of any one of SEQ ID NOs: 32, 58, 75-76, 101-110, 187-199, 207-296, and 301-311: or a nucleic acid sequence that is at least 70%, at least 75%, at least 80%, at least 85%, at least 90%, at least 95%, or at least 99% identical to the nucleic acid sequence of any one of SEQ ID NOs: 32, 58, 75-76, 101-110, 187-199, 207-296, and 301-311.

In some embodiments, the gRNAs comprise a scaffold or backbone sequence that is 100% (e.g., at least 80%, at least 85%, at least 90%, at least 95%, at least 98%, at least 99%, at least 99.9%) identical to the nucleic acid sequence of SEQ ID NO: 20 (5′-cuucacugauaaaguggagaaccgcuucaccaaaagcugucccuuaggggauuagaacuugagugaaggugggcugcuu gcaucagccuaaugucgagaagugcuuucuucggaaaguaacccucgaaacaaauucauuucaagaaagugaaugaagga augcaac-3′). In some embodiments, the gRNAs provided herein comprise a backbone sequence with one or more substitutions relative to a wild-type Nme2Cas9 gRNA. In some embodiments, the portions of the gRNA other than the backbone sequence do not comprise any substitutions relative to a wild-type Nme2Cas9 gRNA.

In another aspect, the present disclosure provides complexes comprising a fusion protein (e.g., any of the fusion proteins provided herein) and a gRNA (e.g., any of the gRNAs provided herein).

In another aspect, the present disclosure provides methods for modifying (e.g., editing) a target nucleic acid molecule comprising contacting the target nucleic acid molecule with any of the fusion proteins provided herein and a guide RNA, or with any of the complexes provided herein. In some embodiments, the target sequence comprises a genomic sequence associated with a disease or disorder (e.g., a point mutation, such as a T→C point mutation or an A→G point mutation).

In another aspect, the present disclosure provides polynucleotides encoding any of the Cas proteins, fusion proteins, guide RNAs, or components of the complexes provided herein. The present disclosure also provides vectors comprising any of the polynucleotides provided herein.

In another aspect, the present disclosure provides kits comprising any of the Cas proteins, fusion proteins, guide RNAs, complexes, polynucleotides, or vectors provided herein.

In another aspect, the present disclosure provides pharmaceutical compositions comprising any of the Cas proteins, fusion proteins, guide RNAs, complexes, polynucleotides, or vectors provided herein, and a pharmaceutically acceptable excipient.

In another aspect, the present disclosure contemplates the use of any of the Cas proteins, fusion proteins, guide RNAs, complexes, polynucleotides, vectors, and pharmaceutical compositions provided herein in the manufacture of a medicament for the treatment of a disease or disorder. In some embodiments, any of the Cas proteins, fusion proteins, guide RNAs, complexes, polynucleotides, vectors, and pharmaceutical compositions provided herein are for use in medicine.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTIONS OF THE DRAWINGS

FIGS. 1A-IE show the development of a function-dependent Cas9 selection and the ePACE platform for automated parallel evolution. FIG. 1A shows an overview of existing Cas9 PACE requiring only PAM binding upstream of a promoter controlling expression of gIII, compared to the sequence-agnostic Cas PACE selection (SAC-PACE) developed in this disclosure, which requires both PAM binding and subsequent base editing. FIG. 1B shows the selection circuit in SAC-PACE. The selection phage (SP) encodes an adenine base editor in place of gIII. In the host cells, an accessory plasmid (AP) contains a cis intein-split gIII, with a linker (31-121 aa) containing stop codons. Correction of the stop codons through recognition of a novel PAM and subsequent base editing results in excision of the cis-intein, production of functional gIII, and phage propagation. FIG. 1C shows the overnight phage propagation assays to test the selection stringency of SAC-PACE with various AP promoter strengths. FIG. 1D provides an overview of ePACE, enabling parallel lagoon evolution of a Cas9 variant on single PAMs (see also FIGS. 14A-18). ePACE is based on the eVOLVER continuous culture platform, adapted to facilitate the automated operation of parallel PACE selections. FIG. 1E shows the overnight propagation assays of wild-type Nme2-ABE8e on two sets of 32 N₃NYN PAMs. Fold-propagation was measured by qPCR and is reflective of the average of two independent biological replicates. The eight CTTAYNA PAMs are excluded as they introduce an additional stop codon in the AP, preventing Cas-dependent propagation.

FIGS. 2A-2G show the evolution of Nme2Cas9 variants with broadened PAM compatibility. FIG. 2A shows the overview of SAC-PACE modifications increasing selection stringency. (Left) original selection scheme (SEQ ID NO: 611); (middle) split SAC-PACE selection in which the expression of TadA8e is placed on a complementary plasmid (CP) in the host cell, enabling tunable control of active enzyme concentration; (right) dual PAM split SAC-PACE selection in which limited active enzyme concentration is coupled with a requirement to edit an additional protospacer and PAM sequence containing a stop codon. In the evolutions described herein, the protospacer was kept constant for multi-site edits. FIG. 2B provides an overview of the evolution campaigns towards Nme2Cas9 variants with N₄CN, or N₄TN PAM compatibility. FIG. 2C describes a summary heat map showing ABE-PPA activity for representative variants across both evolutionary trajectories. Values plotted are raw observed % A•T-to-G•C conversion for one replicate of each base editor. FIG. 2D shows the mutation overview of the eNme2-C variant, mapped onto the crystal structure of wild-type Nme2Cas9 (PDB: 6JE3), mutated positions are indicated by asterisks. The inset shows the wild-type PAM and PAM-interacting residues (D1028, R1033), with evolved mutations listed. FIG. 2E shows the mutation overview of the eNme2-T.1 and eNme2-T.2 variants, mapped onto the crystal structure of wild-type Nme2Cas9 (PDB: 6JE3), positions mutated in both variants are indicated by daggers (t), while mutations unique to eNme2-T.1 (SEQ ID NO: 2) are indicated by asterisks, and mutations unique to eNme2-T.2 are indicated by diamonds. The insets show the wild-type PAM and PAM-interacting residues (D1028, R1033), along with novel mutations listed. FIG. 2F depicts summary dot-plots which show the progression of mammalian cell adenine base editing activity at eight N₄CN PAM-containing sites for representative variants from the N₄CN evolution trajectory. FIG. 2G depicts summary dot-plots showing the progression of mammalian cell adenine base editing activity at eight N₄TN PAM-containing sites for representative variants from the N₄TN evolution trajectory. In FIG. 2E and FIG. 2G, each point represents the average editing of n=3 independent biological replicates measured at the maximally edited position within each given genomic site. Mean±SEM is shown and reflects the average activity and standard error of the pooled genomic site averages. ns, p>0.05; *, p≤0.05; **, p≤0.01, ***, p≤50.001, ****, p≤0.0001. p-values determined by Sidak's multiple comparisons test following ordinary one-way ANOVA.

FIGS. 3A-3I show the characterization of evolved Nme2Cas9 variants in mammalian cells. FIG. 3A shows an overview of PAM-matched sites used to compare eNme2Cas9 variants to SpRY and SpRY-HF1 (SEQ ID NO: 612). FIG. 3B depicts summary dot plots showing the activity of eNme2-C-ABE8e compared to SpRY-ABE8e and SpRY-HF1-ABE8e at 14 PAM-matched NCN/N₄CN sites in HEK293T cells. Left-most data represent a summary of all 14 sites, and subsequent columns represent a subdivision into specific PAMs. FIG. 3C depicts summary dot plots showing the activity of eNme2-T.1-ABE8e and eNme2-T.2-ABE8e compared to SpRY-ABE8e and SpRY-HF1-ABE8e at eight PAM-matched NTN/N₄TN sites in HEK293T cells. FIG. 3D depicts summary dot plots showing the activity of eNme2-C-BE4 compared to SpRY-BE4 and SpRY-HF1-BE4 at eight PAM-matched NCN/N₄CN sites in HEK293T cells. FIG. 3E depicts summary dot plots showing the activity of eNme2-C nuclease and eNme2-C.NR (SEQ ID NO: 4) nuclease compared to SpRY nuclease and SpRY-HF1 nuclease at eight PAM-matched NCN/N₄CN sites in HEK293T cells. FIG. 3F shows an overview of protospacer-matched sites used to compare the DNA specificity of eNme2Cas9 variants against SpRY and SpRY-HF1 (From left to right, SEQ ID NOs: 613-614). FIG. 3G depicts heat maps showing off-target adenine base editing activity (top) or off-target indel formation (bottom) at computationally-determined off-targets for two sites in HEK293T cells for eNme2-C-ABE8e and eNme2-C.NR (SEQ 1D NO: 4) nuclease compared to SpRY and SpRY-HF1 adenine base editor and nuclease variants. The left-most column represents on-target activity. Values are listed for any sites at which ≥1% editing or indels was observed, and represent the average of n=3 independent biological replicates. FIG. 3H shows a percentage of on-target GUIDE-seq reads identified at four protospacer matched sites for eNme2-C nuclease, eNme2-C.NR (SEQ ID NO: 4) nuclease, SpRY nuclease, and SpRY-HF1 nuclease. Total reads for the given nuclease are listed above each bar. (i) Total putative off-target sites identified by GUIDE-seq for eNme2-C nuclease, eNme2-C.NR (SEQ ID NO: 4) nuclease, SpRY nuclease, and SpRY-HF1 nuclease at four protospacer-matched sites. For (FIGS. 3B-3E), each point represents the average editing of n=3 independent biological replicates measured at the maximally edited position within each given genomic site. Mean±SEM is shown and reflects the average activity and standard error of the pooled genomic site averages.

FIGS. 4A-4F show the generalizability of eNme2-C-ABE8e across different cell types and targets. FIG. 4A depicts summary dot plots showing the activity of eNme2-C-ABE8e compared to SpRY-ABE8e and SpRY-HF1-ABE8e at 15 PAM-matched NCN/N₄CN sites in HUH7 cells. Left-most data represent a summary of all 15 sites, and subsequent columns represent a subdivision into specific PAMs. FIG. 4B depicts summary dot plots showing the activity of eNme2-C-ABE8e compared to SpRY-ABE8e and SpRY-HF1-ABE8e at 18 PAM-matched NCN/N₄CN sites in U2OS cells. Left-most data represent a summary of all 18 sites, and subsequent columns represent a subdivision into specific PAMs. For FIGS. 4A and 4B, each point represents the average editing of n=3 independent biological replicates measured at the maximally edited position within each given genomic site. Mean±SEM is shown and reflects the average activity and standard error of the pooled genomic site averages. FIG. 4C shows eNme2-C-ABE8e compared to SpRY-ABE8e and SpRY-HF1-ABE8e at eight PAM-matched NCN/N₄CN sites in HDFa cells. Bars represent mean±SEM of n=3 independent biological replicates, with individual values shown as dots. FIG. 4D shows the ClinVar identified SNPs that can be targeted with an eNme2-C-ABE8e (right) or eNme2-C-BE4 (left). FIG. 4E shows the installation of a disease-relevant D674G mutation in the RBM20 gene (SEQ ID NO: 622). Tiled guides were used to install the mutation either with eNme2-C-ABE8e or SpRY-ABE8e (see Table 2 for sgRNA sequences). Bars represent mean±SEM of n=3 independent biological replicates, with individual values shown as dots. FIG. 4F shows conversion of the sickle-cell disease-causing HBB E6V mutation to the benign E6A (Makassar hemoglobin) allele using either SpCas-NRCH-ABE8e or eNme2-C-ABE8e (SEQ ID NO: 598). Bars represent mean±SEM of n=3 independent biological replicates, with individual values shown as dots.

FIGS. 5A-5C show the validation of the sequence-agnostic Cas (SAC-PACE) PACE selection. FIG. 5A depicts data from the overnight propagation assay to test the requirements of active intein splicing and stop codons to turn on or off, respectively, the SAC-PACE circuit. Inactive intein was generated by introducing the CIA mutation in the C-intein and the positive control (+ctrl) was a host strain containing pJC175e. FIG. 5B shows data from the overnight propagation assay to test the linker length limitations of SAC-PACE, OT phage did not contain Nme2-ABE8e or TadA8e. FIG. 5C shows data from the overnight propagation assay to test the relative activity of Nme2-ABE8e phage when the target adenines within the stop codons are placed at different locations in the 23 nucleotide Nme2Cas9 protospacer (counting the PAM as positions 24-29). For FIG. 5A-5C, Mean±SEM is shown and are representative of n=2 independent biological replicates. Fold-propagation is calculated as the ratio of titer after overnight propagation over inoculating titer.

FIGS. 6A-6E shows the base editing dependent PAM profiling assay (BE-PPA). FIG. 6A shows the schematic of BE-PPA constructs. A BE-expressing plasmid (BP) containing the base editor to be evaluated was cloned along with a library plasmid (LP) containing a target protospacer and target base (adenine or cytosine for ABE-PPA or CBE-PPA, respectively) flanked by a library of PAMs of interest. FIG. 6B shows the BE-PPA workflow. A cell line containing the BP is first generated, then the LP is electroporated into that cell line before base editor expression is induced. Induced cells are grown for 22-36 hours (with dilution after 24 hours if necessary), before plasmid DNA is harvested and sequenced by high-throughput sequencing. FIG. 6C shows a comparison of the BE-PPA assay against existing mammalian cell base editing PAM profiling. Each point represents 1 of 64 NNN PAMs, normalized to the activity of the highest PAM for BE2 (rAPOBEC1-dSpCas) along the x-axis in BE-PPA or for BE4max along the y-axis for the previously assessed mammalian library. All points reflect the average normalized activity of n=2 independent biological replicates. The line reflects a simple OLS regression, with the R-squared value shown. FIGS. 6D-6E depict heat maps showing ABE-PPA activity of (FIG. 6D) wild-type Nme2-ABE8e and (FIG. 6E) representative clones from ePACE1-3 on the set of 256 N₃NNNN PAMs (PAM positions 1-3 fixed, see Table 1). Values are raw % A•T-to-G•C conversion observed for one replicate of each editor.

FIGS. 7A-7E show the mutation tables and representative activity of ePACE4 evolved Nme2Cas9 variants. FIGS. 7A-7C show the genotypes of individually sequenced plaques following ePACE4, with positions varying from wild-type displayed. Clones evolved on different PAMs are delineated by a bold line. Mutations that had previously appeared in ePACE1 and ePACE2 are outlined with dashed or solid boxes, respectively, while novel mutations are shown without any outline. FIG. 7D is a heat map showing ABE-PPA activity of representative clones from ePACE4 on the 16 combinations of PAM positions 5 and 6 (N₄NN Values are raw % A•T-to-G•C conversion observed for one replicate of each editor and are listed in each cell for the N₄CN PAMs, with values above 70% A•T-to-G•C conversion colored white. FIG. 7E shows the ABE-PPA activity in (FIG. 7D) pooled and segregated by mutation position. Each column depicts the impact of a given position, when mutated, on ABE-PPA activity at each of the four PAM groups (N₅A, N₅C, N₅G, N₅T) (see Example 2). Values are normalized against the highest activity within each set of PAMs. Only positions that were observed to be mutated more than once in (FIGS. 7A-7C) were included in the analysis.

FIGS. 8A-8E show the N₄CN activity, editing window, and preferred spacer length of eNme2-C-ABE8e. FIG. 8A shows adenine base editing activity of eNme2-C-ABE8e at 33 N₃NC PAM-containing sites in HEK293T cells. Mean±SEM is shown and reflects the average activity and standard error of n=3 replicates at the maximally edited position within each genomic site. The site that exhibited <1% base editing activity (line shown) that was excluded in subsequent analyses is italicized. FIG. 8B shows the pooled adenine base editing activity of eNme2-C-ABE8e from FIG. 8A. Left: all 32 sites with base editing >1% for eNme2-C-ABE8e: right: sites pooled by PAM position 6 identity. Each point represents the average editing of n=3 independent biological replicates measured at a given genomic site. Mean±SEM is shown and reflects the average activity and standard error of the pooled genomic site averages. FIG. 8C and FIG. 8D show the editing windows of eNme2-C-ABE8e (FIG. 8C) and Nme2-ABE8e (FIG. 8D) reflective of pooled adenine base editing activity at all 23 protospacer positions (PAM counted as positions 24-29). Each point represents the % A•T-to-G•C conversion observed for an adenine present in one of the 32 protospacers, normalized to the highest editing observed within that protospacer. Italicized positions were not present in any protospacers evaluated. Mean±SEM is shown and reflects the average normalized activity and standard error at all observed adenines at that position. FIG. 8E displays the adenine base editing activity of eNme2-C-ABE8e as a function of protospacer length (between 26-20 nt) at three different genomic sites in HEK293T cells. Each point represents the average of n=3 independent biological replicates observed for a given protospacer length at one genomic site, normalized to the protospacer length with the highest base editing activity for that site. Mean±SEM is shown and reflects the average normalized activity and standard error of the pooled averages at the observed sites. For FIG. 8B, ****, p≤0.0001. p-value determined by unpaired Student's t-test.

FIGS. 9A-9D show the mutation table and representative activity of ePACE5 evolved Nme2Cas9 variants. FIGS. 9A-9C depict the genotypes of individually sequenced plaques following ePACE5, with positions varying from wild-type displayed (from top to bottom of FIG. 9A, SEQ ID NOs are 16, 300, 300, 300, 300, 299, 299, 299, 299, 300, 300, 300, 300, 298, 298, 298, 298, 298, 297, 297, 297, 297, 214, 214, 214, 214, 15, 300, 300, 300, 300, 300, 300, 300, 300, 300, 300, 300, 300, 300, 300, 300, 300, 300, 300, 300, 300, 300, 300, 300, 300). Clones evolved on different PAMs are delineated by a bold line. Mutations that had previously appeared in ePACE1, ePACE2, or ePACE3 are outlined with dashed, solid, or dotted boxes respectively, while novel mutations are shown without any outline. Positions that were unable to be called due to low sequencing quality are denoted by a “-”. FIG. 9D is a heat map showing ABE-PPA activity of representative clones from ePACE5 on the 16 combinations of PAM positions 5 and 6 (N₄NN) Values are raw % A•T-to-G•C conversion observed for one replicate of each editor and are listed in each cell for the N₄TN PAMs, with values above 70% A•T-to-G•C conversion colored white.

FIGS. 10A-10D show the N₄TN activity, editing window, and preferred spacer length of eNme2-T.1-ABE8e and eNme2-T.2-ABE8e. FIG. 10A shows the adenine base editing activity of eNme2-T.1-ABE8e and eNme2-T.2-ABE8e at 16 N₃NTN PAM-containing sites in HEK293T cells. Mean±SEM is shown and reflects the average activity and standard error of n=3 independent biological replicates at the maximally edited position within each genomic site. The six sites that exhibited <1% base editing activity for either variant (line shown) that were excluded in subsequent analyses are italicized. FIG. 10B shows the pooled adenine base editing activity of eNme2-T.1-ABE8e and eNme2-T.2-ABE8e from FIG. 10A. Left: all 10 sites; right: sites pooled by PAM position 6 identity. Each point represents the average of n=3 independent biological replicates measured at the maximally edited position within each given genomic site. Mean±SEM is shown and reflects the average activity and standard error of the pooled genomic site averages. FIG. 10C shows the editing window of eNme2-T.1-ABE8e (top) or eNme2-T.2-ABE8e (bottom) reflective of pooled adenine base editing activity at all 23 protospacer positions (PAM counted as positions 21-26) of the 10 sites shown in FIG. 10A. Each point represents the % A•T-to-G•C conversion observed for an adenine that was present in one of the 10 protospacers, normalized to the highest editing observed within that protospacer. Mean±SEM is shown and reflects the average normalized activity and standard error at all observed adenines at that position. FIG. 10D shows the adenine base editing activity of eNme2-T.1-ABE8e (top) or eNme2-T.2-ABE8e (bottom) as a function of protospacer length (between 26-20 nt) at three different genomic sites in HEK293T cells. Each point represents the average of n=3 independent biological replicates observed for a given protospacer length at one genomic site, normalized to the protospacer length with the highest base editing activity for that site. Mean*SEM is shown and reflects the average normalized activity and standard error of the pooled averages at the observed sites. For FIG. 10B, **, p≤0.01. p-values determined by individual unpaired Student's t-tests comparing Nme2-ABE8e to either eNme2-T.1-ABE8e or eNme2-T.2-ABE8e.

FIGS. 11A-11F show eNme2 variants compared to SpRY and SpRY-HF1 in HEK293T cells. FIG. 11A shows the adenine base editing activity of eNme2-C-ABE8e compared to SpRY-ABE8e and SpRY-HF1-ABE8e at 14 NCN/N₄CN PAM-matched sites in HEK293T cells (pooled data in FIG. 3B). FIG. 11B shows the adenine base editing activity of eNme2-T.1-ABE8e and eNme2-T.2-ABE8e compared to SpRY-ABE8e and SpRY-HF1-ABE8e at eight NTN/N₄TN PAM-matched sites in HEK293T cells (pooled data in FIG. 3C). FIG. 11C shows the cytosine base editing activity of eNme2-C-BE4 compared to SpRY-BE4 and SpRY-HF1-BE4 at eight NCN/N₄CN PAM-matched sites in HEK293T cells (pooled data in FIG. 3D). FIG. 3D shows the nuclease activity of eNme2-C nuclease and eNme2-C.NR (SEQ ID NO: 4) nuclease compared to SpRY nuclease and SpRY-HF1 nuclease at eight NCN/N₄CN PAM-matched sites in HEK293T cells (pooled data in FIG. 3E). For FIGS. 11A-11D, the mean±SEM is shown and reflects the average activity and standard error of n=3 independent biological replicates measured at the maximally edited position (if applicable) within each given genomic site. FIG. 11E shows the pooled adenine base editing activity of eNme2-C-ABE8e compared to eNme2-C.NR-ABE8e or adenine base editors generated from reversion mutations at each of the eight RuvC/HNH domain mutations in eNme2-C at eight genomic sites in HEK293T cells. FIG. 11F shows the pooled nuclease activity of eNme2-C nuclease compared to eNme2-C.NR (SEQ ID NO: 4) nuclease or nuclease-active variants generated from reversion mutations at each of the eight RuvC/HNH domain mutations in eNme2-C at eight genomic sites in HEK293T cells. For FIGS. 11E-11F, each point represents the average of n=3 independent biological replicates measured at the maximally edited position within each given genomic site in HEK293T cells. Mean±SEM is shown and reflects the average activity and standard error of the pooled genomic site averages.

FIGS. 12A-12E show GUIDE-Seq identified off-targets of Nme2 variants compared to SpRY and SpRY-HF1. FIG. 12A show the on-target indel formation of wild-type Nme2 nuclease, eNme2-C nuclease, and eNme2-C.NR (SEQ ID NO: 4) nuclease compared to SpRY nuclease and SpRY-HF1 nuclease at each of the four protospacer-matched sites that were subsequently evaluated in GUIDE-Seq. Each bar represents the observed indel formation of one replicate in U2OS cells. FIGS. 12B-12E show GUIDE-Seq identified off-targets and associated read counts for Nme2 variants (top) or SpRY variants (bottom) at Site 3 (FIG. 12B), Site 4 (FIG. 12C), Site 5 (FIG. 12D), and Site 6 (FIG. 12E). For FIGS. 12B-12E, from top to bottom, SEQ ID NOs are 321-395, 396-449, 450-541, and 542-594, respectively. The on-target protospacer is marked by a black dot for each site. Off-target thresholds were set at 8 mismatches with and NNN PAM for SpRY variants or 11 mismatches with an NNNNNN PAM for Nme2 variants).

FIGS. 13A-13B show eNme2-C-ABE8e activity in other human cell types. FIG. 13A show the adenine base editing activity of eNme2-C-ABE8e compared to SpRY-ABE8e and SpRY-HF1-ABE8e at 15 NCN/N₄CN PAM-matched sites in HUH7 cells (pooled data in FIG. 4A). FIG. 13B shows the adenine base editing activity of eNme2-C-ABE8e compared to SpRY-ABE8e and SpRY-HF1-ABE8e at 18 NCN/N₄CN PAM-matched sites in U20S cells (pooled data in FIG. 4B). For FIGS. 13A-13B, the mean±SEM is shown and reflects the average activity and standard error of n=3 independent biological replicates measured at the maximally edited position within each given genomic site.

FIGS. 14A-14D provide a high level description of ePACE components. FIG. 14A shows the photograph of ePACE, consisting of an eVOLVER continuous culture unit with custom vial caps, fluidics unit with a set of slow (˜1 ml/m, pump heads shown above the label “Media/Efflux Pumps”) and fast (˜1 ml/s, pump heads shown under the label “Media/Efflux Pumps”) pump arrays for vial-to-vial/media pumping and waste pumping respectively, Integrated Peristaltic Pump (IPP) device for chemical inducer pumping (˜0.5 ul/s), and a multi-channel pressure regulator for powering IPP devices and pressurizing inducer bottles. FIG. 14B provides a diagram of fluidics for a single ePACE chemostat/lagoon pair. FIG. 14C is a photograph of custom vials and caps designed for ePACE, labeled for a typical setup. Caps are designed to be used with hypodermic needles, but can also be used with other types of tubing. FIG. 14D is a diagram of volume levels for each input/output (I/O) port on the caps with different length needles. In ePACE, the efflux needle is set to 31 ml and 9 ml for the chemostat and lagoon, respectively (underlined).

FIGS. 15A-15C shows the IPP characterization. FIG. 15A is a diagram of IPP functionality. Three valves in series are sequentially opened and closed to induce a peristaltic effect on the flow line. A single set of control lines can be used to pump many channels in parallel. FIG. 15B shows the valve geometry effects on achievable flow rates. Error bars represent the standard deviation over three measurements on a single channel of a single device. FIG. 15C shows three IPP devices, each with three parallel channels with linked control lines, were run continuously for 168 hours at 10 Hz. Every 24 hours, the devices were briefly stopped and flow rate measurements were taken across the device performance range at 10 Hz, 5 Hz, 1 Hz, and 0.1 Hz. Devices were then restarted at 10 Hz immediately after measurements were taken. Error bars represent the standard deviation of measurements taken over the three channels for a given measurement on a single device.

FIGS. 16A-16D show the eVOLVER pressure regulator characterization. FIG. 16A shows a diagram and photo of an 8-channel PID controlled pressure regulator. FIG. 16B shows the comparison of pressures over 24 hours of PID controlled pressure to a manually set valve, both initial set at 1.5 psi (left) and also depicts a simplified electrical schematic of eVOLVER pressure regulator (right). Each proportional valve is controlled via pulse-width modulation (PWM) using a standard eVOLVER PWM board. A single PWM board can control 16 valves simultaneously, enabling control of eight individual pressure lines. Electrical pressure gauge readouts are connected to a standard eVOLVER analog-to-digital (ADC) converter. Both PWM and ADC boards are connected to a SAMD21 Arduino microcontroller which controls valve open/closeness and reads data from the gauges. The microcontroller receives commands from and sends data to the eVOLVER via serial communication protocol. FIG. 16C depicts a schematic of pressure regulation for ePACE. The IPP devices are powered by 8 psi provided by the pressure regulator and standard lab bench vacuum. Inducer bottles receive 1.5 psi. FIG. 16D shows a comparison of flow rates between media bottles with varying volumes of media while pressurized and un-pressurized.

FIGS. 17A-17B show ePACE validation on two-hybrid Maltose Binding Protein (MBP) selection. FIG. 17A provides a diagram of two-hybrid MBP selection. Upon proper folding of MBP, a T7 RNA Polymerase is recruited to transcribe gill. FIG. 17B shows mutation tables of negative control WT MBP and structurally defective MBP after 120 hours of ePACE. MBP G32+I33S shows converging mutations at residues clustered around the monobody-MBP interaction interface (D32G, A63T, R66L), previously observed in PACE1.

FIGS. 18A-18B show the flow rate schedule and titers for ePACE1. SP containing wild-type, full-length Nme2-ABE8e were first diversified in E. coli host cells containing pJC175e2 and MP62, isolated, then seeded into ePACE1 (eight chemostats, one lagoon each targeting each of the eight N₃YTN PAMs). Flow rate stringency for each PAM is shown in the plots, as are resulting titers (measured by qPCR). If lagoons were reseeded with starting phage, the timepoint is indicated by a circle. The N₃TTA lagoon failed prematurely due to a pump failure in the ePACE setup. LOD=limit of detection of qPCR titering, as set by the titer corresponding to the C_{q} for which the qPCR primers had been observed to amplify.

FIGS. 19A-19B show the mutation table and representative activity of ePACE1 evolved Nme2Cas9 variants. FIG. 19A shows the genotypes of individually sequenced plaques following ePACE1, with positions varying from wild-type displayed. Clones evolved on different PAMs are delineated by a bold line. FIG. 19B shows the adenine base editing activity of a representative ePACE1 clone (E1-2-ABE8e) at eight N₃NCN PAM-containing sites and eight N₄TN PAM-containing sites in HEK293T cells. Mean±SEM are shown and are representative of n=3 independent biological replicates measured at the maximally edited position within each given genomic site.

FIGS. 20A-20B show the flow rate schedule and titers for ePACE2. SP previously isolated from ePACE1 lagoons evolved on N₃TTC and N₃CTC PAMs were pooled and reseeded into ePACE2 (eight chemostats, two lagoons each targeting each of the eight N₃YTN PAMs). Flow rate stringency for each PAM is shown in the plots, as are resulting titers (measured by qPCR). LOD=limit of detection of qPCR titering, as set by the titer corresponding to the C_{q} for which the qPCR primers had been observed to amplify.

FIGS. 21A-21C show the identification of ePACE2 selection cheating. FIG. 21A shows a representative agarose gel of PCR products amplifying the target insert from individual ePACE2 late timepoint SP plaques. The expected insert size for Nme2ABE8e is ˜4.5 kb (left, starting SP), whereas multiple recombinant bands appeared for ePACE2 evolved SP. FIG. 21B depicts the sanger sequencing partially mapping the recombinant bands from FIG. 21A onto the gVI coding sequence, the unaligned sequence to the left maps to the gII-containing AP sequence (from top to bottom. SEQ ID NOs are 595, 596, 597). FIG. 21C shows the nucleotide sequence homology between the coding sequence of gIV (where recombination was seen) and the gill coding sequence present on the AP, aligned nucleotides highlighted in black (from top to bottom, SEQ ID NOs are 599-602).

FIGS. 22A-22D show the mutation table and representative activity of Epace2 evolved Nme2Cas9 variants. FIGS. 22A-22C show the genotypes of individually sequenced plaques following Epace2, with positions varying from wild-type displayed. Clones evolved on different PAMs are delineated by a bold line. Mutations that had previously appeared in Epace1 are outlined with dashed boxes, while novel mutations are indicated with no outline. From top to bottom of FIG. 22B, SEQ ID NOs are 603, 604, 605, 605, 606, 606, 606, 603, 606, 607, 608, 608, 609, 603, 603, 610, 603, 603, 603, 603, 603). FIG. 22D shows the adenine base editing activity of a representative ePACE2 clone (E2-12-ABE8e) at eight NaCN PAM-containing sites and eight N₄TN PAM-containing sites in HEK293T cells. Mean±SEM are shown and are representative of n=3 independent biological replicates measured at the maximally edited position within each given genomic site. Mean±SEM is shown and reflects the average activity and standard error of the pooled genomic site averages.

FIG. 23 shows the validation of the split-SAC-PACE selection with different TadABE8e variants. Overnight propagation assay to test the activity of the split-SAC-PACE selection with different TadA8e variants. Each TadA8e variant was fused to the N-terminal half of an intein (gp41-8N) and placed on a complementary plasmid (CP) in host cells. FL-Nme2ABE8e phage contained full-length, active Nme2ABE8e, and OT phage did not contain Nme2Cas9, intein, or TadA8e. Mean±SEM are shown and are representative of n=2 independent biological replicates. Fold-propagation is calculated as the ratio of phage titer after overnight propagation over inoculating titer.

FIGS. 24A-24B show the flow rate schedule and titers for ePACE3. SP from ePACE1 sequenced SP from ePACE2 were pooled and recloned into the split-SAC-PACE phage architecture (SP404, Table 3), then seeded into ePACE3 (seven chemostats, two lagoons each targeting each of the eight N₃YTN PAMs; N₃TTA was excluded due to a cloning error). Flow rate stringency for each PAM is shown in the plots, as are resulting titers (measured by qPCR). The N₃TTC and N₃TTT lagoons were started late due to slow initial host cell growth. LOD=limit of detection of qPCR titering, as set by the titer corresponding to the C_{q} for which the qPCR primers had been observed to amplify.

FIGS. 25A-25C show the mutation table and representative activity of ePACE3 evolved Nme2Cas9 variants. FIGS. 25A-25B show the genotypes of individually sequenced plaques following ePACE3, with positions varying from wild-type displayed. Clones evolved on different PAMs are delineated by a bold line. Mutations that had previously appeared in ePACE1 are outlined with dashed boxes. Mutations that had previously appeared in ePACE2 are indicated with no outline. Novel mutations are outlined with solid boxes. FIG. 25C show the adenine base editing activity of a representative ePACE3 clone (E3-18-ABE8e) at eight N₄TN PAM-containing sites in HEK293T cells. Mean±SEM are shown and are representative of n=3 independent biological replicates measured at the maximally edited position within each given genomic site. Mean±SEM is shown and reflects the average activity and standard error of the pooled genomic site averages.

FIGS. 26A-26B show the PANCE dilution schedule and titers for N1. SP containing wild-type split-dNme2Cas9 or pooled ePACE1/ePACE2 split-Nme2Cas9 were first diversified in E. coli host cells containing pJC175e2 and MP62, isolated, then seeded into PANCE1 (N1, 6 chemostats of each of six N₃WTD PAMs, where W=A or T and D=A, G, or T; 4 replicates). FIG. 26A shows the passage stringency schedule and resulting titers (measured by qPCR) for replicates 1 and 2 (top) or replicates 3 and 4 (bottom). Passages were done after 16-24 hours for all passages. For some passages, some conditions were passaged uniquely to others or in a different host cell line, and these changes are listed in the Notes column. Empty boxes represent titers that were not measured, or the PAM had not yet been included. All N₃ACD PAMs were unable to support phage propagation, which retroactively was discovered to be attributable to an AP design error (see Example 2). LOD=limit of detection of qPCR titering, as set by the titer corresponding to the C_{q} for which the qPCR primers had been observed to amplify. FIG. 26B states the PANCE conditions used for N1.

FIGS. 27A-27B show flow rate schedule and titers for ePACE4. SP from N1, passage 20, were combined and seeded into corresponding PAMs in ePACE4 (six chemostats, two lagoons each of the three N₃A_D PAMs, where D=A, G, or T, three lagoons each of the three N₃TCD PAMs). N1 replicates 1 and 2 were pooled into “Lagoon 1” lagoons. N1 replicates 3 and 4 were pooled into “Lagoon 2” lagoons, and all N1 replicates were pooled into any “Lagoon 3” lagoons. The N₃ACD PAMs all washed out, which retroactively was discovered to be attributable to an AP design error (see Example 2). Flow rate stringency for each PAM is shown in the plots, as are resulting titers (measured by qPCR). LOD=limit of detection of qPCR titering, as set by the titer corresponding to the C_{q} for which the qPCR primers had been observed to amplify.

FIGS. 28A-28B show the PANCE dilution schedule and titers for N2. SP containing wild-type split-dead Nme2Cas9 (dNme2Cas9), pooled ePACE1/ePACE2 split-Nme2Cas9, or pooled ePACE3 split-Nme2Cas9 were first diversified in E. coli host cells containing pJC175e2 and MP6², isolated, then seeded into PANCE2 (N2, eight chemostats of each of eight N₃YTN PAMs, where Y=C or T, three replicates). FIG. 28A shows the passage stringency schedule and resulting titers (measured by qPCR) for replicates 1-3. Passages were done after 16-24 hours for all passages. For some passages, some conditions were passaged uniquely to others or in a different host cell line, and these changes are listed in the Notes column. Empty boxes represent titers that were not measured or the PAM had not yet been included. LOD=limit of detection of qPCR titering, as set by the titer corresponding to the C_{q} for which the qPCR primers had been observed to amplify. FIG. 28B states the PANCE conditions used for N2.

FIGS. 29A-29B show the flow rate schedule and titers for ePACE5. SP from N2 replicate 3, passage 7, were combined and seeded into corresponding PAMs in ePACE5 (eight chemostats, two lagoons each of the eight N₃YTN PAMs, where Y=C or T). Flow rate stringency for each PAM is shown in the plots, as are resulting titers (measured by qPCR). As most lagoons were unable to support consistent phage propagation, the timepoints used for isolating and sequencing phage are marked by a black box. LOD=limit of detection of qPCR titering, as set by the titer corresponding to the C_{q} for which the qPCR primers had been observed to amplify.

FIGS. 30A-30B show the in silico prediction of off-target sites with ≤3 mismatches for a 20-nt or 23-nt protospacer. FIG. 30A shows the count of genome-wide (GRCh38) sites with 0, 1, 2, or 3 mismatches to a 20-nt (SpCas9) or 23-nt (Nme2Cas9) protospacer identified with CHOPCHOPv3³. Mean±SEM representing identified off-targets at six randomly selected 20-nt or 23-nt protospacers are shown. FIG. 30B is a table listing the number of identified sites with the corresponding number of mismatches to a 20-nt or 23-nt protospacer at six randomly selected genomic sites (see Table 2).

FIG. 31 shows the PAM activity of Cas variants across different selection schemes.

FIG. 32 shows a schematic of the dual positive/negative sequence-agnostic Cas PACE (SAC-PACE) selection circuit. Included are specific illustrations of the selection phage (SP), a complementary plasmid (CP), an accessory plasmid (AP), a negative accessory plasmid (APₙ) and a mutagenesis plasmid (MP).

FIG. 33 shows a graph of validation of an orthogonal in cis intein pair for use in the APₙ. Overnight propagation assay to test the ability of three different intein pairs to splice and enable functional gIII-neg expression from the APₙ in a base editing-dependent or independent manner. Mean±SEM is shown and are representative of n=2 independent biological replicates. Fold-propagation is calculated as the ratio of titer after overnight propagation over inoculating titer.

FIG. 34 shows a graph of validation of the triple PAM, split SAC-PACE APs. Overnight propagation assay to test the ability of previously evolved Nme2Cas9 phage (E5 phage) to propagate on APs containing three copies of the target PAM requiring correction of three stop codons. E5 pooled phage were previously evolved on N₃NTN PAMs using the dual PAM, split SAC-PACE selection. The off-target phage (OT) contains neither a variant of Nme2Cas9 nor an adenosine deaminase. Mean±SEM is shown and are representative of n=2 independent biological replicates. Fold-propagation is calculated as the ratio of titer after overnight propagation over inoculating titer.

FIG. 35A shows a schematic of the layout of the N1 PANCE selection campaigns towards N₃TTN-specific variants of Nme2Cas9. PANCE conditions used to evolve Nme2Cas9 towards N₃TTN-specific activity. Four PAMs were targeted in the positive selection (triple PAM, split base editor), multiplexed with three different negative selection stringencies (no APₙ, pro5 expression of gII-neg, or proC expression of gII-neg). All conditions were run in triplicate.

FIG. 35B shows a graph of overnight propagation of E5 phage on dual positive/negative SAC-PACE selections with varying negative selection stringencies. Overnight propagation of previously evolved Nme2Cas9 (E5 phage) on the 12 different selection stringencies targeted in N1. Mean±SEM is shown and are representative of n=3 independent biological replicates. Fold-propagation is calculated as the ratio of titer after overnight propagation over inoculating titer.

FIG. 36 show graphs of dilution schedule and titers for N1. Briefly, SP containing pooled E5 phage were first diversified in E. coli host cells containing pJC175e105 and MP6105, isolated, then seeded into N1. Dilution schedules for each condition is shown in the plots, as are resulting titers (measured by qPCR). If lagoons needed to be reseeded, the passage is highlighted with a circle.

FIG. 37 shows a mutation table of N1 variants evolved towards N₃TTG specificity. Genotypes of individually sequenced plaques following 13 passages in N1 of the 3 conditions targeting N₃TTG PAMs in the positive selection. For clarity, only mutations in the PID are shown, with positions varying from wild-type displayed. Clones evolved without negative selection are outlined with a solid line; clones evolved with medium stringency negative selection are outlined with a dashed line, and clones evolved with high stringency negative selection are outlined with a dotted line.

FIGS. 38A-38B show heat maps of ABE-PPA activity of N1 evolved Nme2Cas9 variants and a graph of on/off-target PAM activity ratio of Nme2ABE8e variants. FIG. 38A shows heat maps showing ABE-PPA activity of N1-5-ABE8e (left), which was evolved without negative selection, and eNme2-N1-21-ABE8e (“N1-21-ABE8e”), which was evolved with high stringency negative selection, on the set of 256 N₃NNNN PAMs (PAM positions 1-3 fixed). Values are raw % A•T-to-G•C conversion observed for one replicate of each editor. FIG. 38B shows a graph of on/off-target PAM activity ratio of N1-5-ABE8e and N1-21-ABE8e. The ratio is calculated by taking the % A•T-to-G•C conversion observed for the positive selection PAM (N₃TTG) divided by the % A•T-to-G•C conversion observed for the negative selection PAM (N₃CCC).

FIG. 39 shows a graph comparing N15-21-ABE8e to eNme2-T.1-ABE8e at N₃NTN PAM sites in HEK293T cells. Adenine base editing activity of the dual positive/negative SAC-PACE evolved N15-21-ABE8e variant compared to a prior, PAM-promiscuous eNme2-T.1-ABE8e variant at six N₃NTN sites in HEK293T cells. Mean±SEM is shown and reflects the average activity and standard error of n=3 independent biological replicates measured at the maximally edited position within each given genomic site.

FIG. 40A shows eNme2-T.1-ABE8e and eNme2-T.2-ABE8e activity at N₄VN PAM sites. (a) Adenine base editing activity of eNme2-T.1-ABE8e and eNme2-T.2-ABE8e at 22 N₃NVN PAM sites in HEK293T cells. Mean±SEM is shown and reflects the average activity and standard error of n=3 replicates at the maximally edited position within each genomic site.

FIG. 40B shows eNme2-T.1-ABE8e and eNme2-T.2-ABE8e activity at N₄VN PAM sites. Adenine base editing activity in (a) pooled by PAM position 5 (N₄NN) identity, also including pooled N₄TN sites from FIG. 10A-10D.

FIGS. 41A-41H show graphs of high-throughput sequencing validation of GUIDE-seq identified off-target activity. High-throughput sequencing in HEK293T cells at the top off target sites nominated by GUIDE-seq for eNme2-C, eNme2-C.NR (SEQ ID NO: 4). SpRY, or SpRY-HF1 nucleases (Table 3). Off-target indel formation by Nme2Cas9, eNme2-C.NR (SEQ ID NO: 4), SpRY, or SpRY-HF1 nuclease at nominated off target sites for the sgRNAs targeting Site 3 (FIG. 41A), Site 4 (FIG. 41B), Site 5 (FIG. 41C), or Site 6 (FIG. 41D). Off-target adenine base editing by Nme2-ABE8e, eNme2-C-ABE8e, SpRY-ABE8e, or SpRY-HF1-ABE8e at nominated off-target sites for the sgRNAs targeting Site 3 (FIG. 41E), Site 4 (FIG. 41F), Site 5 (FIG. 41G), or Site 6 (FIG. 41H). Mean±SEM is shown and reflects the average activity and standard error of n=3 independent biological replicates measured at the maximally edited position within each given genomic site. On-target activity is shown at the left-most entry for each site.

FIG. 42A and FIG. 42B are graphs of off-target adenine base editing at in silico-predicted off-target sites for SpRY-ABE8e, SpRY-HF1-ABE8e, eNme2-T.1-ABE8e and eNme2-T.2-ABE8e. (FIG. 42A) Off-target adenine base editing by SpRY-ABE8e, SpRY-HF1-ABE8e, eNme2-T.1-ABE8e and eNme2-T.2-ABE8e at 12 computationally determined off-targets of a protospacer-matched sgRNA (Site 7) or (FIG. 42B) 23 computationally determined off-targets of a protospacer matched sgRNA (Site 8). Mean±SEM is shown and reflects the average activity and standard error of the of n=3 independent biological replicates measured at the maximally edited position within each given genomic site. On-target activity is shown at the left-most entry for each site.

FIG. 43 shows a graph of dose-dependent adenine base editing activity in primary human dermal fibroblasts. Dose titration of mRNA encoding Nme2-ABE8e, eNme2-CABE8e, SpRY-ABE8e, or SpRY-HF1-ABE8e electroporated into primary human dermal fibroblasts together with synthetic guide RNA targeting either the GCT-2 or CCG-1 site. Mean±SEM is shown and reflects the average activity of one biological replicate measured for each dose targeting the two different endogenous genomic sites.

FIG. 44A and FIG. 44B show graphs of off-target adenine base editing at in silico-predicted off-target sites for SpCas9-NRCH and eNme2-C sgRNAs targeting the HBB sickle-cell disease mutation. (FIG. 44A) Off-target adenine base editing by eNme2-C-ABE8e at nine computationally nominated off-target sites for the sgRNA targeting the HBB sickle-cell disease mutation. (FIG. 44B) Off-target adenine base editing by SpCas9-NRCH-ABE8e at 11 computationally nominated off-target sites for the sgRNA targeting the HBB sickle-cell disease mutation. Mean*SEM is shown and reflects the average activity and standard error of the of n=3 independent biological replicates measured at the maximally edited position within each given genomic site. On-target activity is shown at the left-most entry for each site.

FIG. 45 is a heat map of ABE-PPA activity of different Nme2A8e variants (wild-type, E1-2AB8e, E2-12A8e, E3-18A8e, eNme2-T.1A8e, eNme2-T2.1A8e).

FIG. 46 is a schematic of a Host E. coli cell used in the negative selection strategy containing the AP, APₙ, MP and CP plasmids and different evolution outcomes. If gill and gIII-neg are expressed, or if gIII-neg only is expressed or no gIII is expressed the phage is not infective. However, if gIII only is expressed the phage is infectious.

FIG. 47 is a schematic of components of an AP and an AP plasmid, showing exemplary nucleotide sequences (from left to right, SEQ ID NOs: 615 and 617) and active PAM site on the APₙ plasmid. Amino acid SEQ ID NO: 616.

FIG. 48 is a schematic of a phage expressing a selection phage (SP) and phage genes (e.g., gill). The phage is shown infecting a Host E. coli cell. The Host E. coli cell contains an AP plasmid and an MP plasmid. From left to right, nucleotide sequences are SEQ ID NOs: 618 and 620; amino acid sequences are SEQ ID NOs: 619 and 621.

FIG. 49 shows a workflow of a high throughput multiplex assay where PANCE (low stringency) on individual PAMs can be performed in parallel. Iteratively pass surviving PAMs are then selected for on PACE (high stringency) using multiplex chemostats.

FIG. 50 shows a workflow of a high throughput multiplex assay where PANCE (low stringency) on individual PAMs can be performed in parallel. Parallelized PACE of multiple individual PAMs are then selected for on PACE (high stringency) using eVOLVER-supported PACE.

FIG. 51A is a graph comparing eNme2-C.NR (SEQ ID NO: 4), eNme2-C, SpRY, SpRY-HF1 nucleases on different PAM sites (e.g., Site 3, Site 4, Site 5, Site 6). The number of GUIDE-seq identified putative off-target sites is shown on the y-axis.

FIG. 51B is a graph showing off-target activity of Nme2-ABE8e, eNme2-C-ABE8e, SpRY-ABE8e, SpRY-HF1-ABE8e, compared to untreated. The % A•T converted to G•C at maximally edited position(s) is shown on the y-axis.

FIG. 52 is a schematic showing the SpCas9 variants and Nme2Cas9 variants and their ability to access different targets.

FIG. 53 are schematics of Cas (e.g., SpCas9, and Nme2Cas9) variants and their accessibility. On the (left panel) is limited accessibility Cas9 variant (e.g., SpCas9) that targets and NGG PAM. Compared to broad genome accessibility of Cas9 variants (e.g., SpCas9 and Nme2Cas9) (middle panel) and optimized, target-specific access Cas variants (right panel).

### DETAILED DESCRIPTION OF CERTAIN EMBODIMENTS

The details of one or more embodiments of the invention are set forth herein. Other features, objects, and advantages of the invention will be apparent from the Detailed Description, Examples, Figures, and Claims. References cited in this application are incorporated herein by reference in their entireties.

Streptococcus pyogenes Cas9 (SpCas9) is a widely-utilized genome-editing tool, but due to its large size, alternative, smaller-sized nucleic acid-programmable DNA-binding proteins are needed for use in genome editing agents, such as base editors. As described herein, the term “genome editing” may refer to conventional CRISPR-Cas9 gene editing that introduces a double-strand break. As described herein, the term “base editing” may refer to genome editing by Cas9 machinery that avoids double-stranded breaks. In some embodiments, any of the disclosed base editors or vectors may comprise a partially inactive Cas9 nickase, such as an Nme2Cas9 nickase (Nme2Cas9n) containing a D16A mutation, fused to an effector domain such as a deaminase. In some embodiments, any of the disclosed base editors or vectors may contain a catalytically inactive Cas9, such as a dead Nme2Cas9 (dNme2Cas9) containing D16A and H588A mutations, fused to an effector domain such as a deaminase. The limitations imposed by PAM restrictions have motivated efforts to engineer or evolve Cas protein variants with broadened or altered PAM compatibility. These approaches have generated variants of the most widely used Cas9 from Streptococcus pyogenes (SpCas9), which offers robust mammalian cell activity and engages sites with NGG PAMs, where N=A, C, G, or T. The wild-type and evolved or engineered variants of SpCas9 described to date can collectively access essentially all purine-containing PAMs and a subset of pyrimidine-containing PAMs.

The present disclosure is based on the directed evolution and engineering of variants of Neisseria meningitidis Cas9 (Nme2Cas9) with improved recognition of non-canonical PAMs in a target nucleic acid molecule (e.g., when used in the context of a base editor). Multiple rounds of eVOLVER-supported phage-assisted continuous evolution (ePACE) and phage-assisted non-continuous evolution (PANCE) of Nme2Cas9 were performed to yield several variants with broader PAM recognition. Furthermore, because Nme2Cas9 is 1082 amino acids long, and therefore small enough to enable design of single-AAV vectors for delivery of various CRISPR-based base editors, the evolved Cas9 variants described herein are useful in various genome editing agents. Thus, the disclosed Cas9 variants are about 275-350 amino acids shorter than other Cas9 proteins, such as SpCas9 (1,082 as vs 1,368 aa), making Nme2Cas9 attractive for delivery applications, such as AAV particle delivery.

Evolved and engineered Cas9 variants have proven critical to therapeutic ex vivo and in vivo precision gene editing. Some genomic loci-especially those with pyrimidine-rich PAM sequences-remain inaccessible by high-activity Cas9 variants. Moreover, engineering broad PAM sequence compatibility can increase off-target activity. Using PANCE and eVOLVER-supported phage-assisted continuous evolution (ePACE), the Nme2Cas9, a compact Cas9 variant, was evolved towards novel, single-nucleotide pyrimidine PAM recognition.

A general selection strategy was developed that required functional editing while allowing the target protospacer and PAM to be fully specified. This selection was applied to evolve four new, high-activity Nme2Cas9 variants. Evolved variants eNme2-C and eNme2-C.NR (SEQ ID NO: 4) enabled efficient base editing and nuclease-mediated indel formation, respectively, at sites containing N₄CN PAMs, where N can be any nucleotide. Variants eNme2-T.1 (SEQ ID NO: 2) and eNme2-T.2 enable adenine base editing at many N₄TN PAM sequences. When compared to SpRY, the only reported Cas protein variant capable of engaging a similar range of pyrimidine PAMs, eNme2-T.1 (SEQ ID NO: 2) and eNme2-T.2 offer alternative access to N₄TN PAM sequences at comparable efficiencies, while eNme2-C and eNme2-C.NR (SEQ ID NO: 4) offer less restrictive PAM requirements, comparable or higher activity in a variety of human cell types, and much lower off-target activity at N₄CN PAM sequences. Together, these evolved Nme2Cas9 variants enable targeting of most pyrimidine-rich PAM sequences, including those poorly accessed by existing Cas proteins, substantially expanding the targeting capabilities of Cas9-based technologies.

The present disclosure also provides systems and methods for SAC-PACE, a directed system for evolution of any Cas ortholog that may be adapted for selection against broad PAM compatibility. The present disclosure also provides systems and methods for ePACE, a platform for automated, massively parallel evolutionary selection of Cas9 proteins that utilizes the millifluidic systems of eVOLVER. The ePACE system was developed based on an eVOLVER continuous culture platform and adapted to facilitate the automated operation of parallel PACE selections. eVOLVER is a multi-objective, inexpensive, do-it-yourself platform of automated culture growth experiments and is constructed using highly modular, open-source wetware, hardware, electronics, and web-based software. As described herein, the sequential actuation of consecutively-arranged pneumatic valves using integrated peristaltic pump “IPP” device(s), and a multi-channel pressure regulator enabled the simultaneous execution of PACE experiments across eight different PAMs (or other selection conditions) in parallel. Moreover, the fabrication method utilizing laser-cut acrylic, silicone elastomer membrane (as opposed to PDMS), and an adhesive bond (as opposed to a thermal or chemical method) brings both the cost of manufacturing the devices and the time down considerably from microfluidics.

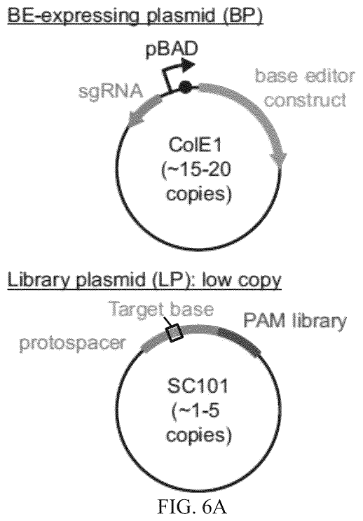

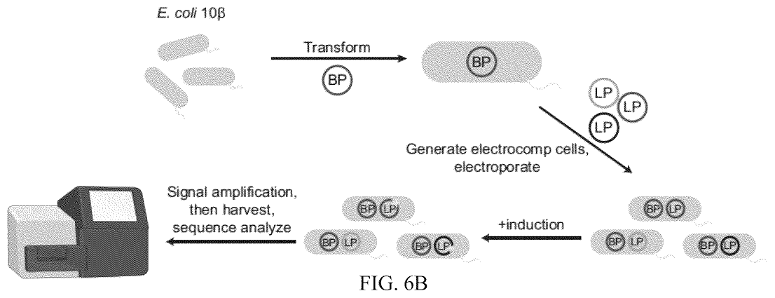

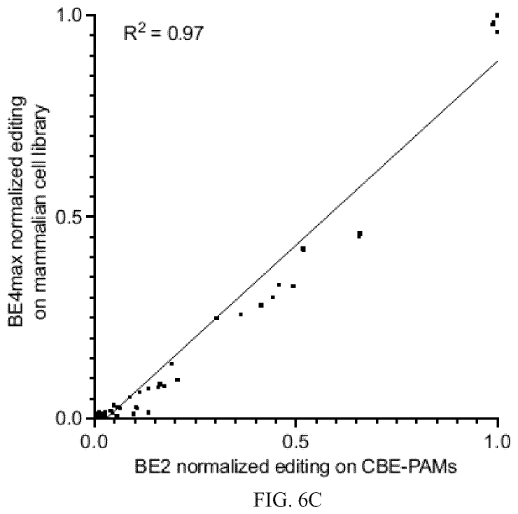

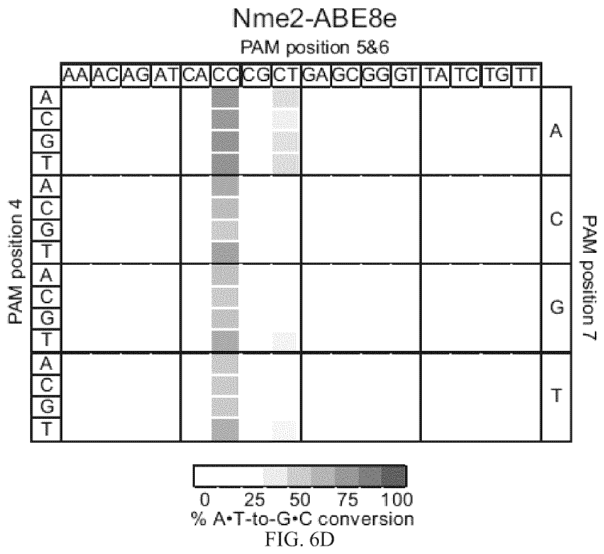

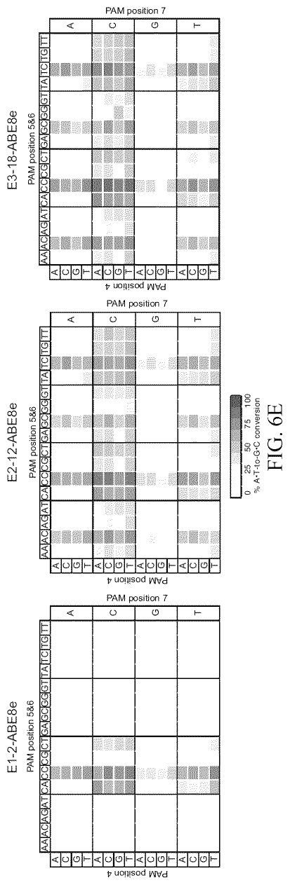

The present disclosure, in some aspects, provides methods for rapid assessment of the PAM specificities of newly evolved Cas9 variants, when these variants are fused to an effector domain in a base editor. As described herein, a “base editing-dependent PAM profiling assay” or “BE-PPA” may describe a high-throughput assay that may be used to thoroughly characterize Cas9 (e.g., Nme2Cas9) variants and guide evolutionary trajectories. In some embodiments, the disclosed BE-PPA assays may involve a protospacer or library of protospacers containing target adenines for adenine base editors (ABE-PPA) or target cytosines for cytosine base editors (CBE-PPA) that is installed upstream of a library of PAM sequences. In some embodiments, the library may be transformed into E. coli (e.g., E. coli 10β) along with a plasmid expressing a base editor of interest. In some embodiments, the BE-expressing plasmid (BP) may comprise a vector (e.g., plasmid) the contains a promoter sequence, a base editor construct and a sgRNA. In some embodiments, the BP may comprise an sgRNA, a promoter, and a base editor construct. In some embodiments, the library plasmid (LP) may contain a protospacer, a target base and a PAM library. For exemplary BP and LP plasmids, see FIG. 6A. In some embodiments, after the BP and LP are both transformed into the into E. coli (e.g., E. coli 10β) cell, induction occurs, followed by signal amplification, harvesting and sequence analysis. In some embodiments, to analyze BE-PPA sequenced files, demultiplexed fastq files may be filtered using the seqkit package/grep function to search for two flank sequences near either end of the amplicon. In some embodiments, for ABE-PPA profiled variants, groups of PAMs may be UMI-tagged, and the specific UMI tag may be used in place of one of the flank sequences. Filtered files may next be binned into individual fastq files per PAM using the same function. In further embodiments, the resulting PAM-specific fastq files may be analyzed using standard CRISPResso2 analysis. Since base editing at each PAM is measured independently of other PAMs, BE-PPA offers greater sensitivity compared to nuclease-based assays. See FIG. 6B for an exemplary BE-PPA workflow. The present disclosure illustrates the design and validation of the BE-PPA profiling method.

Thus, the present disclosure provides Cas9 protein variants comprising one or more amino acid substitutions relative to wild-type Nme2Cas9 (SEQ ID NO: 5). Fusion proteins comprising the Cas protein variants described herein are also provided by the present disclosure. Further provided herein are methods for editing a target nucleic acid using the Cas proteins and fusion proteins provided herein. The present disclosure also provides complexes, polynucleotides, vectors, host cells, kits, and pharmaceutical compositions comprising any of the disclosed fusion proteins, and guide RNAs, complexes, kits and pharmaceutical compositions for base editing methods using any of the disclosed fusion proteins. Some aspects of the present disclosure relate to methods for editing a target nucleic acid molecule comprising contacting the nucleic acid molecule with a complex comprising a fusion protein and a guide RNA (gRNA). In some embodiments, the contacting is performed in vitro or in vivo. In some embodiments, the contacting is performed in vitro. In some embodiments, the contacting is performed in vivo. In some embodiments, the contacting is performed in a subject. In some embodiments, the subject has been diagnosed with a disease or disorder. In some embodiments, the target sequence comprises a genomic sequence associated with a disease or disorder. In some embodiments, the target sequence comprises a point mutation associated with a disease or disorder. In some embodiments, the point mutation comprises a T to C point mutation associated with a disease or disorder. In some embodiments, the point mutation comprises a C to T point mutation associated with a disease or disorder. In some embodiments, the point mutation comprises an A to G point mutation associated with a disease or disorder. In some embodiments, the point mutation comprises an G to A point mutation associated with a disease or disorder. In further embodiments, the step of editing the target nucleic acid results in correction of the point mutation.

### Definitions

As used herein and in the claims, the singular forms “a,” “an,” and “the” include the singular and the plural unless the context clearly indicates otherwise. Thus, for example, a reference to “an agent” includes a single agent and a plurality of such agents.

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (TTRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

rAAV particles may comprise a nucleic acid vector (e.g., a recombinant genome), which may comprise at a minimum: (a) one or more heterologous nucleic acid regions comprising a sequence encoding a protein or polypeptide of interest (e.g., any of the disclosed fusion proteins) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

As used herein, the term “adenosine deaminase” or “adenosine deaminase domain” refers to a protein or enzyme that catalyzes a deamination reaction of an adenosine (or adenine). The terms are used interchangeably. In certain embodiments, the disclosure provides base editors comprising one or more adenosine deaminase domains. For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminase can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

The term “base editor (BE),” as used herein, refers to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, T to G). In some embodiments, the base editor is capable of deaminating a base within a nucleic acid such as a base within a DNA molecule. In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017 and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28:152(5):1173-83 (2013), each of which are incorporated by reference herein).

In some embodiments, a base editor is a macromolecule or macromolecular complex that results primarily (e.g., more than 80%, more than 85%, more than 90%, more than 95%, more than 99%, more than 99.9%, or 100%) in the conversion of a nucleobase in a polynucleic acid sequence into another nucleobase (i.e., a transition or transversion) using a combination of 1) a nucleotide-, nucleoside-, or nucleobase-modifying enzyme and 2) a nucleic acid binding protein that can be programmed to bind to a specific nucleic acid sequence.

In some embodiments, the base editor comprises a DNA binding domain (e.g., a programmable DNA binding domain such as a dCas9 or nCas9) that directs it to a target sequence. In some embodiments, the base editor comprises a nucleobase modifying enzyme fused to a programmable DNA binding domain (e.g., a dCas9 or nCas9). A “nucleobase modifying enzyme” is an enzyme that can modify a nucleobase and convert one nucleobase to another (e.g., a deaminase such as a adenosine deaminase). Base editors that carry out certain types of base conversions (e.g., adenosine (A) to guanine (G), C to G) are contemplated.

In some embodiments, a base editor converts an A to G. In some embodiments, the base editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known natural adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in PCT Application PCT/US2017/045381, filed Aug. 3, 2017, which published as WO 2018/027078, and PCT Application No. PCT/US2019/033848, filed May 23, 2019, which published on Nov. 28, 2019 as WO 2019/226953, U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019: International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Application No. PCT/US2019/033848, filed May 23, 2019, which published as Publication No. WO 2019/226593 on Nov. 28, 2019; International Publication No. WO 2018/0176009, published Sep. 27, 2018, International Publication No. WO 2020/041751, published Feb. 27, 2020; International Publication No. WO 2020/051360, published Mar. 12, 2020; International Patent Publication No. WO 2020/102659, published May 22, 2020; International Publication No. WO 2020/086908, published Apr. 30, 2020: International Publication No. WO 2020/181180, published Sep. 10, 2020; International Publication No. WO 2020/214842, published Oct. 22, 2020; International Publication No. WO 2020/092453, published May 7, 2020; International Publication No. WO2020/236982, published Nov. 26, 2020; International Application No. PCT/US2020/624628, filed Nov. 25, 2020: International Publication No. WO 2021/158921, published Aug. 12, 2021; International Application No. PCT/US2022/073781, filed Jul. 15, 2022; PCT Publication No. WO 2020/236982, published Nov. 26, 2020; and PCT Publication No. WO 2021/108717, published Jun. 3, 2021, the contents of each of which are incorporated herein by reference in their entireties.

As used herein, a “cytidine deaminase” encoded by the CDA gene is an enzyme that catalyzes the removal of an amine group from cytidine (i.e., the base cytosine when attached to a ribose ring) to uridine (C to U) and deoxycytidine to deoxyuridine (C to U). Exemplary cytidine deaminases include members of the apolipoprotein B mRNA editing enzyme, catalytic polypeptide (APOBEC) family. A non-limiting example of a cytidine deaminase from the APOBEC family is APOBEC1. Another example is AID (“activation-induced cytosine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytosine deaminase will cause the insertion of “A” instead of a “G” during cellular repair and/or replication processes. Since the adenine “A” pairs with thymine “T”, the cytosine deaminase in coordination with DNA replication causes the conversion of a C G pairing to a T A pairing in the double-stranded DNA molecule.

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., et al. Science 337:816-821 (2012), the entire contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, Neisseria meningitidis, S. pyogenes and S. thermophilus (e.g., Nme2Cas9, StCas9, or St1Cas9). Exemplary Cas9 orthologs of this disclosure include Nme2Cas9 and variants thereof. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In some embodiments the Cas9 is from N. meningitidis [Nme1Cas9 and Nme2Cas9, Type II-C]. The PAM-interacting (PI) domains of Nme1Cas9 and Nme2Cas9 are highly diverged (52% identity) even though the other portions of the proteins are >98% identical. This divergence leads to distinct PAM specificities: N₄GAYW/N₄GYTT/N₄GTCT for Nme1Cas9, and N₄CC for Nme2Cas9 [PAM sequences are 5′ to 3′ on the non-target strand (NTS)]. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)), and the mutation D16A completely inactivates the nuclease activity of NmeCas9 (Edraki et al. Molecular Cell 73, 714-726). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., Nme2Cas9 of SEQ ID NO: 5). In some embodiments, the Cas9 variant may have 1, 2, 3, 4, 5, 6, 7, 8, 9, 10, 11, 12, 13, 14.15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., Nme2Cas9 of SEQ ID NO: 5). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., Nme2Cas9 of SEQ ID NO: 5). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., Nme2Cas9 of SEQ ID NO: 5).

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of the D16A and H588A mutations in wild-type Nme2Cas9 amino acid sequence, one of D10A or H840A mutations in the wild-type S. pyogenes Cas9 amino acid sequence, or a D10A mutation in the wild-type S. aureus Cas9 amino acid sequence, may be used to form the nCas9.

The term “cDNA” refers to a strand of DNA copied from an RNA template. cDNA is complementary to the RNA template.

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase Ill.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara 1., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine.

The deaminases described herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

The term “off-target editing frequency,” as used herein, refers to the number or proportion of unintended base pairs, e.g., DNA base pairs, that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. The number of off-target DNA edits may be measured by techniques known in the art, including high-throughput screening of sequencing reads, EndoV-Seq, GUIDE-Seq, CIRCLE-Seq, and Cas-OFFinder. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and Illumina-based next-generation genome sequencing (NGS).

The term “on-target editing,” as used herein, refers to the introduction of intended modifications (e.g., deaminations) to nucleotides (e.g., adenine) in a target sequence, such as using the base editors described herein. The term “off-target DNA editing,” as used herein, refers to the introduction of unintended modifications (e.g. deaminations) to nucleotides (e.g. adenine) in a sequence outside the canonical base editor binding window (i.e., from one protospacer position to another, typically 2 to 8 nucleotides long). Off-target DNA editing can result from weak or non-specific binding of the gRNA sequence to the target sequence. As used herein, the term “bystander editing” refers to synonymous off-target point mutations at nucleobases that are near (proximate to) the target base and do not change the outcome of the intended editing method.

As used herein, the terms “purity” and “product purity” of a base editor refer to the mean the percentage of edited sequencing reads (reads in which the target nucleobase has been converted to a different base) in which the intended target conversion occurs (e.g., in which the target A, and only the target A, is converted to a G). See Komor et al., Sci Adv 3 (2017).

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

As used herein, an “effector domain” refers to a molecule (e.g., a protein) that regulates a biological activity and/or is capable of modifying a biological molecule (e.g., a protein, or a nucleic acid such as DNA or RNA). In some embodiments, the effector domain is a protein. In some embodiments, the effector domain is capable of modifying a protein (e.g., a histone on a nucleic acid molecule). In some embodiments, the effector domain is capable of modifying DNA (e.g., genomic DNA). In some embodiments, the effector domain is capable of modifying RNA (e.g., mRNA). In some embodiments, the effector molecule is a nucleic acid editing domain. In some embodiments, the effector molecule is capable of regulating an activity of a nucleic acid (e.g., transcription, and/or translation). Exemplary effector domains include, without limitation, a deaminase, a nuclease, a nickase, a recombinase, a methyltransferase, a methylase, an acetylase, an acetyltransferase, a transcriptional activator, or a transcriptional repressor domain. In some embodiments, the effector domain is a nucleic acid editing domain. Some aspects of the disclosure provide fusion proteins comprising a Cas protein domain and a nucleic acid editing domain. In some embodiments, the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

The term “effective amount,” as used herein, refers to an amount of a biologically active agent that is sufficient to elicit a desired biological response. For example, in some embodiments, an effective amount of a base editor may refer to the amount of the editor that is sufficient to edit a target site nucleotide sequence, e.g., a genome. In some embodiments, an effective amount of a base editor described herein, e.g., of a base editor comprising a nickase Cas9 domain and a guide RNA may refer to the amount of the base editor that is sufficient to induce editing of a target site specifically bound and edited by the base editor. As will be appreciated by the skilled artisan, the effective amount of an agent, e.g., a base editor, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The term “functional equivalent” refers to a second biomolecule that is equivalent in function, but not necessarily equivalent in structure to a first biomolecule. For example, a “Cas9 equivalent” refers to a protein that has the same or substantially the same functions as Cas9, but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally occurring, engineered, circular permutant, mutated, or synthetic version of protein X which bears an equivalent function.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof fused to an adenosine deaminase. Any of the proteins described herein may be produced by any method known in the art. For example, the proteins described herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “guide nucleic acid” or “napDNAbp-programming nucleic acid molecule” or equivalently “guide sequence” refers to one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system. Chemically, guide nucleic acids can be all RNA, all DNA, or a chimeric of RNA and DNA. The guide nucleic acids may also include nucleotide analogs. Guide nucleic acids can be expressed as transcription products or can be synthesized.

As used herein, a “guide RNA”, or “gRNA,” refers to a synthetic fusion of the endogenous bacterial crRNA and tracrRNA that provides both targeting specificity and a scaffold and/or binding ability for Cas9 nuclease to a target DNA. This synthetic fusion does not exist in nature and is also commonly referred to as an sgRNA. However, the term, guide RNA, also embraces equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein. In addition, methods for designing appropriate guide RNA sequences are provided herein.

A guide RNA is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence for the guide RNA. Functionally, guide RNAs associate with Cas9, directing (or programming) the Cas9 protein to a specific sequence in a DNA molecule that includes a sequence complementary to the protospacer sequence for the guide RNA.

As used herein, a “spacer sequence” is the sequence of the guide RNA (˜20 nts in length) which has the same sequence (with the exception of uridine bases in place of thymine bases) as the protospacer of the PAM strand of the target (DNA) sequence, and which is complementary to the target strand (or non-PAM strand) of the target sequence.

As used herein, the “target sequence” refers to the ˜20 nucleotides in the target DNA sequence that have complementarity to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA, and the protospacer is DNA).

As used herein, the terms “guide RNA core,” “guide RNA scaffold sequence” and “backbone sequence” refer to the sequence within the gRNA that is responsible for Cas9 binding, it does not include the 20 bp spacer sequence that is used to guide Cas9 to target DNA.

The term “host cell,” or a “population of host cells” as used herein, refers to a cell or group of cells that can host and replicate a vector encoding a base editor, guide RNA, and/or combination thereof, as described herein. In some embodiments, host cells are mammalian cells, such as human cells. Provided herein are methods of transducing and transfecting a host cell, such as a human cell, e.g., a human cell in a subject, with one or more vectors provided herein, such as one or more viral (e.g., rAAV) vectors provided herein.

It should be appreciated that any of the base editors, guide RNAs, and or combinations thereof, described herein may be introduced into a host cell in any suitable way, either stably or transiently. In some embodiments, a base editor may be transfected into the host cell. In some embodiments, the host cell may be transduced or transfected with a nucleic acid construct that encodes a base editor. For example, a host cell may be transduced (e.g., with a viral particle encoding a base editor) with a nucleic acid that encodes a base editor, or the translated base editor. As an additional example, a host cell may be transfected with a nucleic acid (e.g., a plasmid) that encodes a base editor or the translated base editor. Such transductions or transfections may be stable or transient. In some embodiments, host cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into host cells through electroporation, transient transfection (e.g., lipofection, such as with Lipofectamine 3000®), stable genome integration (e.g., piggybac), viral transduction, or other methods known to those of skill in the art.

Also provided herein are host cells for packaging of viral particles. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. In some embodiments, the host cell is a eukaryotic cell, for example, a yeast cell, an insect cell, or a mammalian cell. The type of host cell, will, of course, depend on the vector employed, and suitable host cell/vector combinations will be readily apparent to those of skill in the art.

The term “linker,” as used herein, refers to a chemical group or a molecule linking two molecules or domains, e.g. dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker, which is 32 amino acids in length. In some embodiments, the linker is a 32-amino acid linker. In other embodiments, the linker is a 30-, 31-, 33- or 34-amino acid linker.

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. Because of their nature, gain-of-function mutations are usually dominant. Many loss-of-function mutations are recessive, such as autosomal recessive.

The term “napDNAbp”, which stands for “nucleic acid programmable DNA binding protein”, refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Cas14a1, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gegoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7); 768-73, which is incorporated herein by reference.

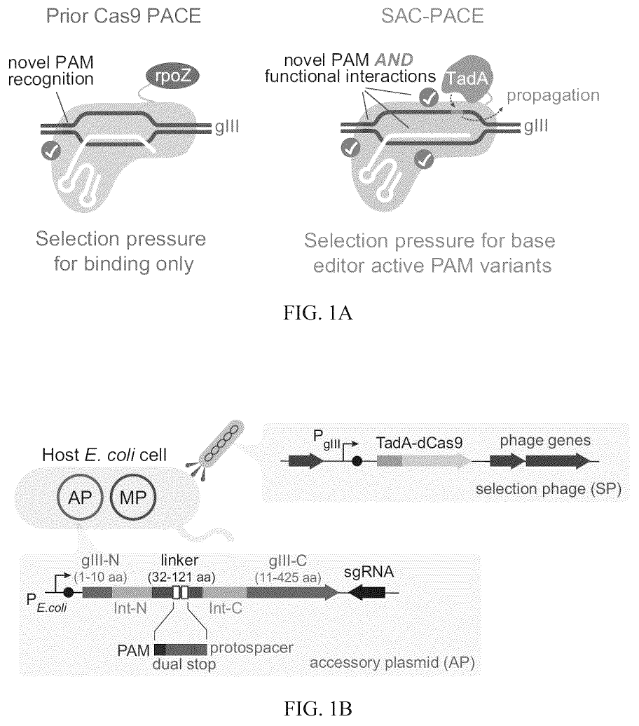

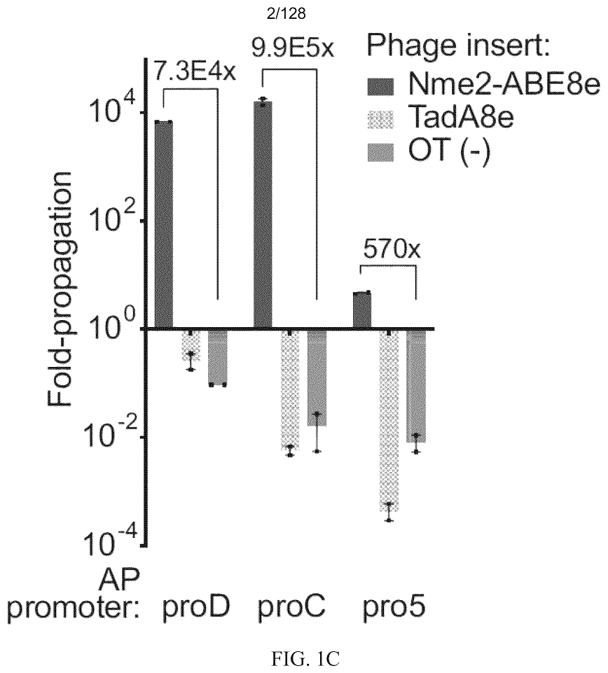

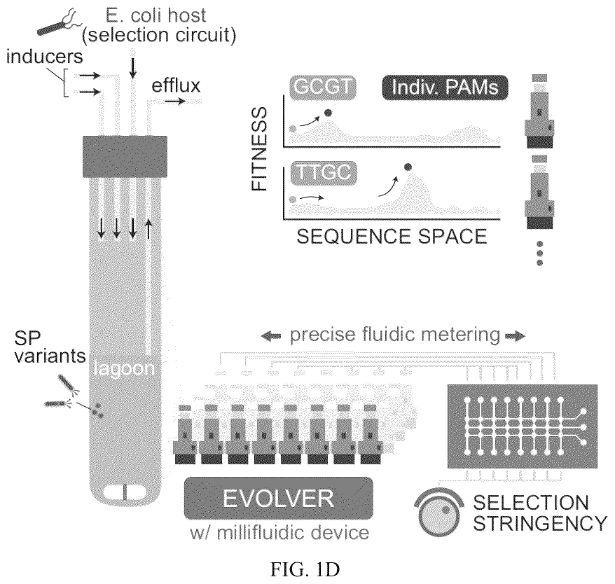

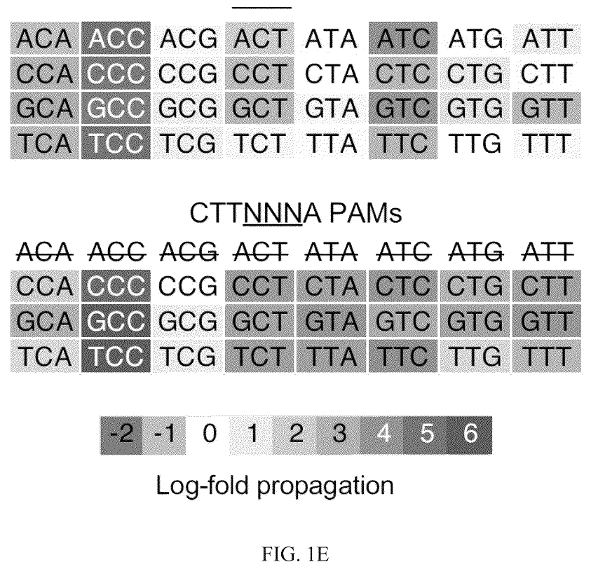

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are incorporated herein by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Nal. Acad. Sci. U.S.A. 98:4658-4663(2001): “CRISPR RNA maturation by trans-encoded small RNA and host factor RNase Ill.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo. J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

The term “nickase” refers to a napDNAbp (e.g., a Cas9) having only a single nuclease activity that cuts only one strand of a target DNA, rather than both strands. Thus, a nickase type napDNAbp does not leave a double-strand break. Exemplary nickases include Nme2Cas9, SpCas9, and SaCas9 nickases. In exemplary embodiments, an Nme2Cas9 variant containing a D16A RuvC-inactivating mutation (the nickase-conferring mutation) is provided. In exemplary embodiments, an Nme2Cas9 variant containing a H588A HNH-inactivating mutation (the nickase-conferring mutation) is provided.

A “nuclear localization signal” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, an NLS is fused to the N-terminus of any of the Cas protein variants provided herein. The term “nucleic acid molecule” as used herein, refers to RNA as well as single and/or double-stranded DNA. Nucleic acid molecules may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, 0(6)-methylguanine, and 2-thiocytidine); chemically modified bases; biologically modified bases (e.g., methylated bases, such as 2′-O-methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

The term “phage-assisted continuous evolution (PACE).” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010: International PCT Application, PCT/US2011/066747, filed Dec. 22, 2011, published as WO 2012/088381 on Jun. 28, 2012; U. S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application. PCT/US2016/027795, filed Apr. 15, 2016, published as WO 2016/168631 on Oct. 20, 2016, the entire contents of each of which are incorporated herein by reference.

The term “promoter” is art-recognized and refers to a nucleic acid molecule with a sequence recognized by the cellular transcription machinery and able to initiate transcription of a downstream gene. A promoter may be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters is inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the disclosure provides vectors with appropriate promoters for driving expression of the nucleic acid sequences encoding the base editors (or one or more individual components thereof). In some embodiments, a promoter may comprise one of the following: a phage shock promoter (psp) sequence, a proD sequence, a proC sequence, or a pro5 sequence, optionally the psp sequence. In some embodiments, the promoter may comprise a phage shock promoter (psp) sequence. In some embodiments, the promoter may comprise a proD sequence. In some embodiments, the promoter may comprise a proC sequence. In some embodiments, the promoter may comprise a pro5 sequence.

As used herein, the term “protospacer” refers to the sequence (e.g., a ˜20 bp sequence) in DNA adjacent to the PAM (protospacer adjacent motif) sequence which shares the same sequence as the spacer sequence of the guide RNA, and which is complementary to the target sequence of the non-PAM strand. The spacer sequence of the guide RNA anneals to the target sequence located on the non-PAM strand. In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the protospacer sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer” (and that the protospacer (DNA) and the spacer (RNA) have the same sequence). Thus, the term “protospacer” as used herein may be used interchangeably with the term “spacer.” The context of the description surrounding the appearance of either “protospacer” or “spacer” will help inform the reader as to whether the term is reference to the gRNA or the DNA sequence. Both usages of these terms are acceptable since the state of the art uses both terms in each of these ways.

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 74, the PAM sequence can be modified by introducing one or more mutations, including (a) D1135V, R1335Q, and T1337R “the VRQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are example are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10(5): 891-899 (which is incorporated herein by reference).

The terms “protein,” “peptide.” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. It should be appreciated that the disclosure provides any of the polypeptide sequences provided herein without an N-terminal methionine (M) residue.

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is a domesticated animal. In some embodiments, the subject is a plant.

The term “target site” refers to a sequence within a nucleic acid molecule that is edited by a base editor (BE) disclosed herein. The term “target site,” in the context of a single strand, also can refer to the “target strand” which anneals or binds to the spacer sequence of the guide RNA. The target site can refer, in certain embodiments, to a segment of double-stranded DNA that includes the protospacer (i.e., the strand of the target site that has the same nucleotide sequence as the spacer sequence of the guide RNA) on the PAM-strand (or non-target strand) and target strand, which is complementary to the protospacer and the spacer alike, and which anneals to the spacer of the guide RNA, thereby targeting or programming a Cas9 base editor to target the target site.

A “transcriptional terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail (signal) appear to be more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

In some embodiments, the transcriptional terminator contains a posttranscriptional response element, a sequence that, when transcribed, creates a tertiary structure enhancing expression. In some embodiments, the posttranscriptional response element is derived from woodchuck hepatitis virus (WHV), i.e., is a WPRE. In some embodiments, the terminator contains the gamma subunit of a WPRE, or a W3, as first reported in Choi, J. H., et al. (2014), Mol. Brain 7: 17, incorporated herein by reference. The WPRE also has alpha and beta subunits. Typically, the posttranscriptional response element is inserted 5′ of the transcriptional terminator. In certain embodiments, the WPRE is a truncated WPRE sequence. In certain embodiments, the WPRE is a full-length WPRE.

Non-limiting examples of transcriptional terminators that may be used in accordance with the present disclosure include transcription terminators (or polyadenylation signals) of the bovine growth hormone gene (bGH), human growth hormone gene (hGH), SV40, CW3, ϕ, or combinations thereof. In exemplary embodiments, the transcriptional terminator is an SV40 polyadenylation signal. In exemplary embodiments, the transcriptional terminator does not contain a posttranscription response element, such as WPRE element. In some embodiments, the termination signal may be a sequence that cannot be transcribed or translated, such as those resulting from a sequence truncation.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transitions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

As used herein, “transversions” refer to the interchange of purine nucleobases for pyrimidine nucleobases, or in the reverse and thus, involve the interchange of nucleobases with dissimilar shape. These changes involve T↔A, T↔G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target DNA molecule. The compositions and methods disclosed herein are also capable of inducing both transitions and transversion in the same target DNA molecule, as well as other nucleotide changes, including deletions and insertions.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. The term also encompasses circular permutants, mutants, truncations, or domains of a reference sequence, and which display the same or substantially the same functional activity or activities as the reference sequence. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property. The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein.

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a fusion protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

The term “vector,” as used herein, refers to a nucleic acid that can be modified to encode a gene of interest and that is able to enter into a host cell and replicate within the host cell, and then transfer a replicated form of the vector into another host cell. Exemplary suitable vectors include viral vectors, such as AAV vectors or bacteriophages and filamentous phage, and conjugative plasmids. Additional suitable vectors will be apparent to those of skill in the art based on the present disclosure.

As used herein the term “wild-type” is a term of the art understood by skilled persons and means the typical form of an organism, strain, gene or characteristic as it occurs in nature as distinguished from mutant or variant forms.

### Cas Variants and Domains

Some aspects of the present disclosure provide nucleic acid-programmable DNA binding proteins (napDNAbps) that exhibit improved activity (e.g., when used for base editing in the context of a fusion protein). In some embodiments, a napDNAbp is a Cas protein (e.g., Nme2Cas9). In some aspects, the present disclosure provides Nme2Cas9 variants that exhibit improved activity (e.g., increased editing efficiency when used, for example, in the context of a base editor fusion protein).

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et aL., Science 337:816-821(2012), the entire contents of which is hereby incorporated by reference.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a napDNAbp that is mutated to with respect to a corresponding wild-type enzyme such that the mutated napDNAbp lacks the ability to cleave one or both strands of a target polynucleotide containing a target sequence. Examples of mutations that render Cas9 a nickase include, without limitation, D16A, D10A, H840A, N854A, and N863A in reference to the canonical SpCas9 sequence, or to equivalent amino acid positions in other Cas9 variants or Cas9 equivalents. In some embodiments, provided herein are Nme2Cas9 nickases, e.g., Nme2Cas9 variants having the RuvC-inactivating D16A mutation. In some embodiments, provided herein are Nme2Cas9 variants that have nickase activity.

The terms “Cas9”, “Cas9 variant” or “Cas9 domain” embrace any Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the base editor (BE) of the disclosure.

The Cas proteins described herein comprise various amino acid substitutions relative to the amino acid sequence of wild-type Nme2Cas9, which is provided below, as SEQ ID NO: 5. The length of this protein is 1082 amino acids.

(SEQ ID NO: 5)
MAAFKPNPINYILGLDIGIASVGWAMVEIDEEENPIRLIDLGVRV
FERAEVPKTGDSLAMARRLARSVRRLTRRRAHRLLRARRLLKREG
VLQAADFDENGLIKSLPNTPWQLRAAALDRKLTPLEWSAVLLHLI
KHRGYLSQRKNEGETADKELGALLKGVANNAHALQTGDFRTPAEL
ALNKFEKESGHIRNQRGDYSHTFSRKDLQAELILLFEKQKEFGNP
HVSGGLKEGIETLLMTQRPALSGDAVQKMLGHCTFEPAEPKAAKN
TYTAERFIWLTKLNNLRILEQGSERPLTDTERATLMDEPYRKSKL
TYAQARKLLGLEDTAFFKGLRYGKDNAEASTLMEMKAYHAISRAL
EKEGLKDKKSPLNLSSELQDEIGTAFSLFKTDEDITGRLKDRVQP
EILEALLKHISFDKFVQISLKALRRIVPLMEQGKRYDEACAEIYG
DHYGKKNTEEKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLVRLNEKGYVE
IDHALPFSRTWDDSFNNKVLVLGSENQNKGNQTPYEYFNGKDNSR
EWQEFKARVETSRFPRSKKQRILLQKFDEDGFKECNLNDTRYVNR
FLCQFVADHILLTGKGKRRVFASNGQITNLLRGFWGLRKVRAEND
RHHALDAVVVACSTVAMQQKITRFVRYKEMNAFDGKTIDKETGKV
LHQKTHFPQPWEFFAQEVMIRVFGKPDGKPEFEEADTPEKLRTLL
AEKLSSRPEAVHEYVTPLFVSRAPNRKMSGAHKDTLRSAKRFVKH
NEKISVKRVWLTEIKLADLENMVNYKNGREIELYEALKARLEAYG
GNAKQAFDPKDNPFYKKGGQLVKAVRVEKTQESGVLLNKKNAYTI
ADNGDMVRVDVFCKVDKKGKNQYFIVPIYAWQVAENILPDIDCKG
YRIDDSYTFCFSLHKYDLIAFQKDEKSKVEFAYYINCDSSNGRFY
LAWHDKGSKEQQFRISTQNLVLIQKYQVNELGKEIRPCRLKKRPP
VR

The present disclosure provides Cas variants comprising an amino acid sequence that is at least 70%, at least 75%, at least 80%, at least 85%, at least 90%, at least 95%, or at least 99% identical to the amino acid sequence of a Cas protein of SEQ ID NO: 5. In some embodiments, the amino acid sequence of the Cas variant comprises at least 1, at least 2, at least 3, at least 4, at least 5, at least 6, at least 7, at least 8, at least 9, at least 10, at least 11, at least 12, at least 15, at least 20, or at least 25 substitutions at positions selected from the group consisting of amino acid residues 6, 33, 47, 63, 68, 104, 116, 123, 152, 154, 221, 260, 263, 303, 396, 413, 427, 451, 452, 460, 484, 520, 629, 646, 674, 696, 711, 720, 724, 758, 765, 767, 769, 771, 816, 821, 844, 859, 865, 932, 940, 951, 1005, 1028, 1029, 1031, 1033, 1044, 1047, 1049, 1056, 1064, and 1075 of the amino acid sequence provided in SEQ ID NO: 5, or corresponding mutations in other Cas homologs. In some embodiments, the amino acid sequence of the Cas variant comprises at least 12, at least 20, at least 26, or at least 28 substitutions at positions selected from the group consisting of amino acid residues 6, 33, 47, 63, 68, 104, 116, 123, 152, 154, 221, 260, 263, 303, 396, 413, 427, 451, 452, 460, 484, 520, 629, 646, 674, 696, 711, 720, 724, 758, 765, 767, 769, 771, 816, 821, 844, 859, 865, 932, 940, 951, 1005, 1028, 1029, 1031, 1033, 1044, 1047, 1049, 1056, 1064, and 1075 of the amino acid sequence provided in SEQ ID NO: 5, or corresponding mutations in other Cas homologs. In some embodiments, the Cas variants comprise substitutions selected from the group consisting of P6X, E33X, E47X, R63X, V68X, K104X, A116X, T123X, D152X, E154X, E221X, F260X, A263X, A303X, T396X, H413X, A427X, D451X, H452X, E460X, A484X, E520X, S629X, R646X, N674X, F696X, G711X, D720X, A724X, 1758X, V765X, H767X, K769X, H771X, S816X, V821X, D844X, 1859X, W865X, E932X, K940X, M951X, K1005X, D1028X, S1029X, N1031X, R1033X, K1044X, Q1047X, R1049X, V1056X, N1064X, and L1075X, relative to the amino acid sequence provided in SEQ ID NO: 5, wherein X represents any amino acid, or corresponding mutations in other Cas homologs. In certain embodiments, the Cas variants comprise substitutions selected from the group consisting of P6S, E33G, E47K, R63K, V68M, K104T, A116T, T123A, D152A, D152N, D152G, E154K, E221D, F260L, A263T, A303S, T396A, H413N, A427S, D451V, H452R, E460A, E460K, A484T, E520A, S629P, R646S, N674S, F696V, G711R, D720A, A724S, 1758V, V765A, H767Y, K769R, H771R, 58161, V821A, D844A, 1859V, W865L, E932K, K940R, M951R, K1005R, D1028N, S1029A, N1031S, R1033N, R10330, R1033Y, K1044R, R1049S, R1049C. Q1047R, V1056A, N1064S. and L1075M, relative to the amino acid sequence of SEQ ID NO: 5, or corresponding mutations in other Cas homologs.

In some embodiments, the Cas variants comprise at least 5, at least 6, at least 7, at least 8, at least 9, at least 10, at least 11, at least 12, at least 15, at least 20, at least 25, at least 26, at least 27, at least 28, or more than 28 substitutions at positions selected from the group consisting of amino acid residues 6, 33, 47, 63, 68, 104, 116, 123, 152, 154, 221, 260, 263, 303, 396, 413, 427, 451, 452, 460, 484, 520, 629, 646, 674, 696, 711, 720, 724, 758, 765, 767, 769, 771, 816, 821, 844, 859, 865, 932, 940, 951, 1005, 1028, 1029, 1031, 1033, 1044, 1047, 1049, 1056, 1064, and 1075 of the amino acid sequence provided in SEQ ID NO: 5.

The provided Cas variants may comprise substitutions at any of the following positions relative to SEQ ID NO: 5: P6, E33, K104, D152, F260, A263, A303, D451. E520, R646, F696, G711.1758, H767, E932. N1031, R1033, K1044, Q1047, and V1056 relative to SEQ ID NO: 5. In some embodiments, the amino acid sequence of the Cas variant comprises any (e.g., at least 1, at least 5, at least 10, at least 12, at least 15, at least 20 or more than 10) of the following substitutions: P6S, E33G, K104T, D152A, F260L, A263T, A303S, D451V, E520A, R646S, F696V, G711R, 1758V, H767Y, E932K, N1031S, R1033G, K1044R, Q1047R, and V1056A. In some embodiments, the amino acid sequence of the Gas variant comprises the 20 following substitutions: P6S, E33G, K104T, D152A, F260L, A263T, A303S, D451V, E520A, R646S, F696V, 0711R. 1758V, H767Y, E932K, N1031S, R1033G, K1044R, Q1047R, and V1056A. In some embodiments, the amino acid sequence of the Cas variant comprises an amino acid sequence having at least 80%, at least 85%, at least 90%, at least 95%, at least 98%, or at least 99% identity to the amino acid sequence of SEQ ID NO: 1. The amino acid sequence of the Cas variant may comprise the amino acid sequence set forth as SEQ ID NO: 1.

In some embodiments, the amino acid sequence of the Cas variant comprises substitutions at any of the following positions: K104, D152, F260, A263, A303, D451, E932, N1031, R1033, K1044, Q1047, and V1056 relative to SEQ ID NO: 5. In some embodiments, the amino acid sequence of the Cas variant may comprise any (e.g., at least 1, at least 5, at least 10, at least 12, or more than 10) of the following substitutions: K104T, D152A, F260L, A263T, A303S, D451V, E932K, N1031S, R1033G, K1044R, Q1047R, and V1056A. In some embodiments, the amino acid sequence of the Cas variant may comprise the following 12 substitutions: K104T, D152A, F260L, A263T, A303S, D451V, E932K, N1031S, R1033G, K1044R, Q1047R, and V1056A. The Cas variant may comprise an amino acid sequence having at least 80%, at least 85%, at least 90%, at least 95%, at least 98%, or at least 99% identity to the amino acid sequence of SEQ ID NO: 4; or may comprise the amino acid sequence set forth as SEQ ID NO: 4.

In some embodiments, the amino acid sequence of the Cas variant comprises substitutions at any of the following positions: E47, V68, T123, D152, E154, T396, H413, A427, H452, E460, A484, S629, N674, D720, V765, H767, H771, V821, D844, 1859, W865, M951, K1005, D1028, S1029, R1033, R1049, and N1064 relative to SEQ ID NO: 5. In some embodiments, the Cas variant comprises any (e.g., at least 1, at least 5, at least 10, at least 12, at least 15, at least 20, at least 25, at least 28, or more than 10) of the following substitutions: E47K, V68M, T123A, D152G, E154K, T396A, H413N, A427S, H452R, E460A, A484T, S629P, N674S, D720A, V765A, H767Y, H771R. V821A, D844A, 1859V, W865L, M951R, K1005R, D1028N, S1029A, R1033Y, R1049S, and N1064S. In some embodiments, the Cas variant comprises the following 28 substitutions: E47K, V68M, T123A, D152G, E154K, T396A, H413N, A427S, H452R, E460A, A484T, S629P, N674S, D720A, V765A, H767Y, H771R, V821A, D844A, 1859V, W865L, M951R. K1005R, D1028N, S1029A, R1033Y, R1049S, and N1064S. The Cas variant may comprise an amino acid sequence having at least 80%, at least 85%, at least 90%, at least 95%, at least 98% or at least 99% identity to the amino acid sequence of SEQ ID NO: 2, or comprise the amino acid sequence set forth as SEQ ID NO: 2.

In some embodiments, the amino acid sequence of the Cas variant comprises substitutions at any of the following positions: E47, R63, V68, A116, T123, D152, E154, E221, T396, H452, E460, N674, D720, A724, K769, S816, D844, E932, K940, M951, K1005, D1028, S1029, R1033, R1049, and L1075 relative to SEQ ID NO: 5. In some embodiments, the amino acid sequence of the Cas variant comprises any (e.g., at least 1, at least 5, at least 10, at least 12, at least 15, at least 20, at least 25, at least 26 or more than 10) of the following substitutions: E47K, R63K, V68M, A116T, T123A, D152N, E154K, E221D, T396A, H452R, E460K, N674S, D720A, A724S, K769R, S816I, D844A, E932K, K940R, M951R, K1005R, D1028N, S1029A, R1033N, R1049C, and L1075M. In some embodiments, the amino acid sequence of the Cas variant comprises the following 26 substitutions: E47K, R63K, V68M, A116T, T123A, D152N, E154K, E221D, T396A, H452R, E460K, N674S, D720A, A724S, K769R, S8161, D844A, E932K, K940R, M951R, K1005R, D1028N, S1029A, R1033N, R1049C, and L1075M. The amino acid sequence of the Cas variant my comprise an amino acid sequence having at least 80%, at least 85%, at least 90%, at least 95%, at least 98% or at least 99% identity to the amino acid sequence of SEQ ID NO: 3; or comprises the amino acid sequence set forth as SEQ ID NO: 3.

In exemplary embodiments, the Cas variant is eNme2-C(SEQ ID NO: 1). In some embodiments, the Cas variant is eNme2-C.NR (SEQ ID NO: 4). In some embodiments, the Cas variant is eNme2-T.1 (SEQ ID NO: 2). In some embodiments, the Cas variant is eNme2-T.2 (SEQ ID NO: 3). In some embodiments, the Cas variant is selected from eNme2E1-2, eNme2E2-12, and eNme2E3-18. The eNme2E1-2, eNme2E2-12, and eNme2E3-18 variants emerged from rounds 1-3 of the ePACE evolution experiments described in the Examples. The eNme2-T.1, eNme2-T.2 (SEQ ID NO: 3), eNme2-C(SEQ ID NO: 1), and eNme2-C.NR (SEQ ID NO: 4) variants emerged from rounds 4 and 5 of these evolutions. In some embodiments, the Cas variant is eNme2-N1-21.

The Cas variants of the disclosure may comprise an amino acid sequence containing at least 80%, 85%, 90%, 92.5%, 95%, 96%, 97%, 98%, or 99% identity to any of SEQ ID NOs: 1-4. The Cas variants of the disclosure may comprise an amino acid sequence containing stretches of at least 50, 100, 150, 200, 250, 300, 400, 500, 600, 700, 750, 800, 850, 900, 950, 1000, 1050, or 1075 consecutive amino acids in common with any of SEQ ID NOs: 1-4.

In some embodiments, the disclosed Cas variants may comprise an amino acid sequence containing 1, 2, 3, 4, 5, 6, 7, 8, 9, 10, 10-15, 15-20, 20-25, 25-30, 30-35, 35-40, 40-45, 45-50, or more than 50 amino acids that differ from the sequences of any of SEQ ID NOs: 1-4.

In some embodiments, the Cas variant is any of SEQ ID NOs: 1-4, provided below. The “e” at the beginning of the Nme2 variants described herein signify an “evolved” Nme2 variant. Amino acid substitutions relative to wild-type Nme2Cas9 are indicated in bolded underline.

eNme2-C:
(SEQ ID NO: 1)
MAAFKSNPINYILGLDIGIASVGWAMVEIDEEGNPIRLIDLGVRV
FERAEVPKTGDSLAMARRLARSVRRLTRRRAHRLLRARRLLKREG
VLQAADFDENGLITSLPNTPWQLRAAALDRKLTPLEWSAVLLHLI
KHRGYLSQRKNEGETAAKELGALLKGVANNAHALQTGDFRTPAEL
ALNKFEKESGHIRNQRGDYSHTFSRKDLQAELILLFEKQKEFGNP
HVSGGLKEGIETLLMTQRPALSGDAVQKMLGHCTLEPTEPKAAKN
TYTAERFIWLTKLNNLRILEQGSERPLTDTERSTLMDEPYRKSKL
TYAQARKLLGLEDTAFFKGLRYGKDNAEASTLMEMKAYHAISRAL
EKEGLKDKKSPLNLSSELQDEIGTAFSLFKTDEDITGRLKDRVQP
EILEALLKHISFDKFVQISLKALRRIVPLMEQGKRYDEACAEIYG
VHYGKKNTEEKIYLPPIPADEIRNPVVLRALSQARKVINGVVRRY
GSPARIHIETAREVGKSFKDRKEIAKRQEENRKDREKAAAKFREY
FPNFVGEPKSKDILKLRLYEQQHGKCLYSGKEINLVRLNEKGYVE
IDHALPFSRTWDDSFNNKVLVLGSENQNKGNQTPYEYENGKDNSR
EWQEFKARVETSRFPSSKKQRILLQKFDEDGFKECNLNDTRYVNR
FLCQFVADHILLTGKGKRRVVASNGQITNLLRGFWRLRKVRAEND
RHHALDAVVVACSTVAMQQKITRFVRYKEMNAFDGKTVDKETGKV
LYQKTHFPQPWEFFAQEVMIRVFGKPDGKPEFEEADTPEKLRTLL
AEKLSSRPEAVHEYVTPLFVSRAPNRKMSGAHKDTLRSAKRFVKH
NEKISVKRVWLTEIKLADLENMVNYKNGREIELYEALKARLEAYG
GNAKQAFDPKDNPFYKKGGQLVKAVRVEKTQKSGVLLNKKNAYTI
ADNGDMVRVDVFCKVDKKGKNQYFIVPIYAWQVAENILPDIDCKG
YRIDDSYTFCFSLHKYDLIAFQKDEKSKVEFAYYINCDSSSGGFY
LAWHDKGSREQRFRISTQNLALIQKYQVNELGKEIRPCRLKKRPP
VR
eNme2-T.1:
(SEQ ID NO: 2)
MAAFKPNPINYILGLDIGIASVGWAMVEIDEEENPIRLIDLGVRV
FKRAEVPKTGDSLAMARRLARSMRRLTRRRAHRLLRARRLLKREG
VLQAADFDENGLIKSLPNTPWQLRAAALDRKLAPLEWSAVLLHLI
KHRGYLSQRKNEGETAGKKLGALLKGVANNAHALQTGDFRTPAEL
ALNKFEKESGHIRNQRGDYSHTFSRKDLQAELILLFEKQKEFGNP
HVSGGLKEGIETLLMTQRPALSGDAVQKMLGHCTFEPAEPKAAKN
TYTAERFIWLTKLNNLRILEQGSERPLTDTERATLMDEPYRKSKL
TYAQARKLLGLEDTAFFKGLRYGKDNAEASTLMEMKAYHAISRAL
EKEGLKDKKSPLNLSSELQDEIGTAFSLFKTDEDIAGRLKDRVQP
EILEALLKNISFDKFVQISLKSLRRIVPLMEQGKRYDEACAEIYG
DRYGKKNTEAKIYLPPIPADEIRNPVVLRALSQTRKVINGVVRRY
GSPARIHIETAREVGKSFKDRKEIEKRQEENRKDREKAAAKFREY
FPNFVGEPKSKDILKLRLYEQQHGKCLYSGKEINLVRLNEKGYVE
IDHALPFSRTWDDSFNNKVLVLGSENQNKGNQTPYEYFNGKDNPR
EWQEFKARVETSRFPRSKKQRILLQKFDEDGFKECNLNDTRYVSR
FLCQFVADHILLTGKGKRRVFASNGQITNLLRGFWGLRKVRAENA
RHHALDAVVVACSTVAMQQKITRFVRYKEMNAFDGKTIDKETGKA
LYQKTRFPQPWEFFAQEVMIRVFGKPDGKPEFEEADTPEKLRTLL
AEKLSSRPEAAHEYVTPLFVSRAPNRKMSGAHKATLRSAKRFVKH
NEKVSVKRVLLTEIKLADLENMVNYKNGREIELYEALKARLEAYG
GNAKQAFDPKDNPFYKKGGQLVKAVRVEKTQESGVLLNKKNAYTI
ADNGDRVRVDVFCKVDKKGKNQYFIVPIYAWQVAENILPDIDCKG
YRIDDSYTFCFSLHRYDLIAFQKDEKSKVEFAYYINCNASNGYFY
LAWHDKGSKEQQFSISTQNLVLIQKYQVSELGKEIRPCRLKKRPP
VR
eNme2-T.2:
(SEQ ID NO: 3)
MAAFKPNPINYILGLDIGIASVGWAMVEIDEEENPIRLIDLGVRV
FKRAEVPKTGDSLAMARKLARSMRRLTRRRAHRLLRARRLLKREG
VLQAADFDENGLIKSLPNTPWQLRATALDRKLAPLEWSAVLLHLI
KHRGYLSQRKNEGETANKKLGALLKGVANNAHALQTGDFRTPAEL
ALNKFEKESGHIRNQRGDYSHTFSRKDLQAELILLFEKQKDFGNP
HVSGGLKEGIETLLMTQRPALSGDAVQKMLGHCTFEPAEPKAAKN
TYTAERFIWLTKLNNLRILEQGSERPLTDTERATLMDEPYRKSKL
TYAQARKLLGLEDTAFFKGLRYGKDNAEASTLMEMKAYHAISRAL
EKEGLKDKKSPLNLSSELQDEIGTAFSLFKTDEDIAGRLKDRVQP
EILEALLKHISFDKFVQISLKALRRIVPLMEQGKRYDEACAEIYG
DRYGKKNTEKKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLVRLNEKGYVE
IDHALPFSRTWDDSFNNKVLVLGSENQNKGNQTPYEYFNGKDNSR
EWQEFKARVETSRFPRSKKQRILLQKFDEDGFKECNLNDTRYVSR
FLCQFVADHILLTGKGKRRVFASNGQITNLLRGFWGLRKVRAENA
RHHSLDAVVVACSTVAMQQKITRFVRYKEMNAFDGKTIDKETGKV
LHQRTHFPQPWEFFAQEVMIRVFGKPDGKPEFEEADTPEKLRTLL
AEKLSIRPEAVHEYVTPLFVSRAPNRKMSGAHKATLRSAKRFVKH
NEKISVKRVWLTEIKLADLENMVNYKNGREIELYEALKARLEAYG
GNAKQAFDPKDNPFYKKGGQLVKAVRVEKTQKSGVLLNKRNAYTI
ADNGDRVRVDVFCKVDKKGKNQYFIVPIYAWQVAENILPDIDCKG
YRIDDSYTFCFSLHRYDLIAFQKDEKSKVEFAYYINCNASNGNFY
LAWHDKGSKEQQFCISTQNLVLIQKYQVNELGKEIRPCRMKKRPP
VR
eNme2-C.NR:
(SEQ ID NO: 4)
MAAFKPNPINYILGLDIGIASVGWAMVEIDEEENPIRLIDLGVRV
FERAEVPKTGDSLAMARRLARSVRRLTRRRAHRLLRARRLLKREG
VLQAADFDENGLITSLPNTPWQLRAAALDRKLTPLEWSAVLLHLI
KHRGYLSQRKNEGETAAKELGALLKGVANNAHALQTGDFRTPAEL
ALNKFEKESGHIRNQRGDYSHTFSRKDLQAELILLFEKQKEFGNP
HVSGGLKEGIETLLMTQRPALSGDAVQKMLGHCTLEPTEPKAAKN
TYTAERFIWLTKLNNLRILEQGSERPLTDTERSTLMDEPYRKSKL
TYAQARKLLGLEDTAFFKGLRYGKDNAEASTLMEMKAYHAISRAL
EKEGLKDKKSPLNLSSELQDEIGTAFSLFKTDEDITGRLKDRVQP
EILEALLKHISFDKFVQISLKALRRIVPLMEQGKRYDEACAEIYG
VHYGKKNTEEKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLVRLNEKGYVE
IDHALPFSRTWDDSFNNKVLVLGSENQNKGNQTPYEYENGKDNSR
EWQEFKARVETSRFPRSKKQRILLQKFDEDGFKECNLNDTRYVNR
FLCQFVADHILLTGKGKRRVFASNGQITNLLRGFWGLRKVRAEND
RHHALDAVVVACSTVAMQQKITRFVRYKEMNAFDGKTIDKETGKV
LHQKTHFPQPWEFFAQEVMIRVFGKPDGKPEFEEADTPEKLRTLL
AEKLSSRPEAVHEYVTPLFVSRAPNRKMSGAHKDTLRSAKRFVKH
NEKISVKRVWLTEIKLADLENMVNYKNGREIELYEALKARLEAYG
GNAKQAFDPKDNPFYKKGGQLVKAVRVEKTQKSGVLLNKKNAYTI
ADNGDMVRVDVFCKVDKKGKNQYFIVPIYAWQVAENILPDIDCKG
YRIDDSYTFCFSLHKYDLIAFQKDEKSKVEFAYYINCDSSSGGFY
LAWHDKGSREQRFRISTQNLALIQKYQVNELGKEIRPCRLKKRPP
VR
eNme2E1-2:
(SEQ ID NO: 11)
MAAFKPNPINYILGLDIGIASVGWAMVEIDEEENPIRLIDLGVRV
FERAEVPKTGDSLAMARRLARSVRRLTRRRAHRLLRARRLLKREG
VLQAADFDENGLIKSLPNTPWQLRAAALDRKLTPLEWSAVLLHLI
KHRGYLSQRKNEGETADKELGALLKGVANNAHALQTGDFRTPAEL
ALNKFEKESGHIRNQRGDYSHTFSRKDLQAELILLFEKQKEFGNP
HVSGGLKEGIETLLMTQRPALSGDAVQKMLGHCTFEPAEPKAAKN
TYTAERFIWLTKLNNLRILEQGSERPLTDTERATLMDEPYRKSKL
TYAQARKLLGLEDTAFFKGLRYGKDNAEASTLMEMKAYHAISRAL
EKEGLKDKKSPLNLSSELQDEIGTAFSLFKTDEDITGRLKDRVQP
EILEALLKHISFDKFVQISLKALRRIVPLMEQGKRCDEACAEIYG
DHYGKKNTEEKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLVRLNERGYVE
IDHALPFSRTWDDSFNNKVLVLGSENQNKGNQTPYEYENGKDNSR
EWQEFKARVETSRFPRSKKQRILLQKFDEDGFKECNLNDTRYVNR
FLCQFVADHILLTGKGKRRVFASNGQITNLLRGFWGLRKVRAEND
RHHALDAVVVACSTVAMQQKITRFVRYKEMNAFDGKTIDKETGKV
LHQKTHFPQPWEFFAQEVMIRVFGKPDGKPEFEEADTPEKLRILL
AEKLSSRPEAVHEYVTPLFVSRAPNRKMSGAHKGTLRSAKRFVKH
NEKISVKRVWLTEIKLADLENMVNYKNGREIELYEALKARLEAYG
GNAKQAFDPKDNPFYKKGGQLVKAVRVEKTQESGVLLNKKNAYTI
ADNGDMVRVDVFCKVDKKGKNQYFIVPIYAWQVAENILPDIDCKG
YRIDDSYTFCFSLHKYDLIAFQKDEKSKVEFAYYSNCDSSNGKFY
LAWHDKGRKEQQFRISTQNLVLIQKYQVNELGKEIRPCRMKKRPP
VR
eNme2E2-12:
(SEQ ID NO: 12)
MAAFKPNPINYILGLDIGIASVGWAMVEIDEEENPIRLIDLGVRV
FKRAEVPKTGDSLAMARRLARSMRRLTRRRAHRLLRARRLLKREG
VLQAADFDENGLIKSLPNTPWQLRAAALDRKLAPLEWSAVLLHLI
KHRGYLSQRKNEGETADKKLGALLKGVANNAHALQTGDFRTPAEL
ALNKFEKESGHIRNQRGDYSHTFSRKDLQAELILLFEKQKEFGNP
HVSGGLKEGIETLLMTQRPALSGDAVQKMLGHCTFEPAEPKAAKN
TYTAERFIWLTKLNNLRILEQGSERPLTDTERATLMDEPYRKSKL
TYAQARKLLGLEDTAFFKGLRYGKDNAEASTLMEMKAYHAISRAL
AKEGLKDKKSPLNLSSELQDEIGTAFSLFKTDEDIAGRLKDRVQP
EILEALLKHISFDKFVQISLKALRRIVPLMEQGKRYDEACAEIYG
DRYGKKNTEEKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLVRLNEKGYVE
IDHALPFSRTWDDSFNNKVLVLGSENQNKGNQTPYEYFNGKDNSR
EWQEFKARVETSRFPRSKKQRILLQKFDEDGFKECNLNDTRYVNR
FLCQFVADHILLTGKGKRRVFASNGQITNLLRGFWGLRKVRAENA
RHHALDAVVVACSTVAMQQKITRFVRYKEMNAFDGKTIDKETGKV
LYRKTLFPQPWEFFAQEVMIRVFGKPDGKPEFEEADTPEKLRTLL
AERLSSRPEAVHEYVTPLFVSRAPNRKMSGAHKATLRSAKRFVKH
NEKISVKRVWLTEIKLADLENMVNYKNGREIELYEALKARLEAYG
GNAKQAFDPKDNPFYKKGGQLVKAVRVEKTQESGVLLNKKNAYTI
ADNGDRVRVDVFCKVDKKGKNQYFIVPIYAWQVAENILPDIDCKG
YRIDDSYTFCFSLHKYDLIAFQKDEKSKVEFAYYINCDSSNGKFY
LAWHDKGSKEQQFRISTQNLVLIQKYQVNELGKEIRPCRLKKRPP
VR
eNme2E3-18:
(SEQ ID NO: 13)
MAAFKPNPINYILGLDIGIASVGWAMVEIDEEENPIRLIDLGVRV
FKRAEVPKTGDSLAMARRLARSMRRLTRRRAHRLLRARRLLKREG
VLQAADFDENGLIKSLPNTPWQLRAAALDRKLAPLEWSAVLLHLI
KHRGYLSQRKNEGETADKKLGALLKGVANNAHALQTGDFRTPAEL
ALNKFEKESGHIRNQRGDYSHTFSRKDLQSELILLFEKQKEFGNP
HVSGGLKEGIETLLMTQRPALSGDAVQKMLGHCTFEPAEPKAAKN
TYTAERFIWLTKLNNLRILEQGSERPLTDTERATLMDEPYRKSKL
TYAQARKLLGLEDTAFFKGLRYGKDNAEASTLMEMKAYHAISRAL
EKEGLKDKKSPLNLSSELQDEIGTAFSLFKTDEDIAGRLKNRVQP
EILEALLKHISFDKFVQISLKALRRIVPLMEQGKRYDEACAEIYG
DCYGKKNTEEKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLVRLNEKGYVE
IDHALPFSRTWDYSFNNKVLVLGSENQNKGNQTPYEYENGKDNSR
EWQEFKARVETSRFPRSKKQRILLQKFDEDGFKECNLNDTRYVNR
FLCQFVADHILLTGKGKRRVFASNGQITNLLRGFWGLRKVRAENA
RHHALDAVVVACSTVAMQQKITRFVRYKETNAFDGKTIDKETGKV
LHQKTHFPQPWEFFAQEVMIRVFGKPDGKPEFEEADTPEKLRTLL
AEKLSSRPEAVHEYVTPLFVSRAPNRKMSGAHKATLRSAKRFVKH
NEKISVKRVWLTEIKLADLENMVNYKNGREIELYEALKVRLEAYG
GNAKQAFDPKDNPFYKKGGQLVKAVRVEKTQESGVLLNKKNAYTI
ADNGDRVRVDVFCKVDKKGKNQYFIVPIYAWQVAENILPDIDCKG
YRIDDSYTFCFSLHKYDLIAFQKDEKSKVEFAYYINCDSSNGKFY
LAWHDKGSKEQQFRISTQNLVLIQKYQVNELGKEIRPCRLKKRPP
VR
eNme2N1-21:
(SEQ ID NO: 14)
MAAFKPNPINYILGLDIGIASVGWAMVEIDEEENPIRLIDLGVRV
FQRAEVPKTGDSLAMARRLARSVRRLTRRRAHRLLRARRLLKREG
VLQAADFDENGLIKSLPHTPWQLRTAALDRKLTPLEWSAVLLHLI
KHRGYLSQRKNEGETADKKLGALLKGVANNAHALQTGDFRTPAEL
ALNKFEKESGHIRNQRGDYSHTFSRKNLQSGLILLFEKQKEFGNP
HVSGGLKEGIETLLMTQRPALSGDAVQKMLGHCTFESAEPKAAKN
TYTAERFIWLTKLNNLRILEQGSERPLTDTERATLMDEPYRKSKL
TYAQARKLLGLEDTAFFKGLRYVKDNAEVSTLMEMKAYHAISRAL
EKEGLKDKKSPLNLSSELQDEIGTAFSLFKTDEDITGRLKDRVQP
EILEALLKHISFDKFVQISLKALRRIVPLMEQGKRYDEACAEIYG
VRYGKKNTEEKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLVRLNEKGYVE
IDHALPFSRTWDDSFNNKVLVLGSENQNKGNQTPYEYFNGKDNSR
EWQEFKARVETSRFPRSKKQRILLQKFDEGGFKECNLNDTRYVNR
FLCQFVADHILLTGKGKRRVFASNGQITNLLRGFWGLRKVRAEND
RHHALDAVVVACSTVAMQQKITRFVRYKEMNAFDGKTIDKEAGKA
LYQKARFPQPWEFFAQEVMIRVFGKPDGKPEFEEADTPEKLRTLL
AERLSSRPEAVHEYVTPLFVSRAPNRKMSGAHKVILRSAKRFVKH
NEKISVKRVWLTEIKLADLENMVNYRNGREIELYEALKARLEAYG
GNAKQAFDPKDNPFYKKGGQLVKAVRVERTQERGVLLNKKNAYTI
ADNGDMVRVDVFCKVGKKGKNQYFIVPIYAWQVAENILPDIDCKG
YRIDDSYTFCFSLHKYDLIAFQKDEKSKVEFAYYSYCHSSSGDFY
LAWHDKGSKEQQFRISTQNLVLIQKYQVNELGKEIRPCRLKKRPL
VR

In some aspects, the Cas proteins (or Cas variants) of the disclosure comprise an amino acid sequence that is at least 70%, at least 75%, at least 80%, at least 85%, at least 90%, at least 95%, at least 98% or at least 99% identical to the amino acid sequence of a Cas protein of SEQ ID NO: 5, wherein the amino acid sequence of the Cas protein comprises at least 1, at least 5, at least 10, at least 12, at least 15, at least 20, or at least 25 substitutions at positions selected from the group consisting of amino acid residues 6, 33, 47, 63, 68, 104, 116, 123, 152, 154, 221, 260, 263, 303, 396, 413, 427, 451, 452, 460, 484, 520, 629, 646, 674, 696, 711, 720, 724, 758, 765, 767, 769, 771, 816, 821, 844, 859, 865, 932, 940, 951, 1005, 1028, 1029, 1031, 1033, 1044, 1047, 1049, 1056, 1064, and 1075 of the amino acid sequence provided in SEQ ID NO: 5. In some embodiments, the Cas protein comprises an amino acid sequence that is not identical to the amino acid sequence of wild-type Nme2Cas9. In some embodiments, the amino acid sequence of the Cas protein comprises at least 1, at least 5, at least 10, at least 12, at least 15, at least 20, or at least 25 substitutions selected from the group consisting of P6X, E33X, E47X, R63X, V68X, K104X, A116X, T123X, D152X, E154X, E221X, F260X, A263X, A303X, T396X, H413X, A427X, D451X, H452X, E460X, A484X, E520X, S629X, R646X, N674X, F696X, G711X. D720X, A724X, 1758X, V765X, H767X, K769X, H771X, S816X, V821X, D844X, 1859X, W865X, E932X, K940X, M951X, K1005X, D1028X, S1029X, N1031X, R1033X, K1044X, Q1047X, R1049X, V1056X, N1064X, and L1075X, relative to the amino acid sequence provided in SEQ ID NO: 5, wherein X represents any amino acid other than the wild type amino acid. In certain embodiments, the amino acid sequence of the Cas protein at least 1, at least 5, at least 10, at least 12, at least 15, at least 20, or at least 25 substitutions selected from the group consisting of P6S, E33G, E47K, R63K, V68M, K104T, A116T, T123A, D152A, D152N, D152G, E154K, E221D, F260L, A263T, A303S, T396A, H413N, A427S, D451V, H452R, E460A, E460K, A484T, E520A, S629P, R646S, N674S, F696V, G711R, D720A, A724S, 1758V, V765A, H767Y, K769R, H771R, S8161, V821A. D844A, 1859V, W865L, E932K, K940R, M951R, K1005R, D1028N, 51029A, N103IS, R1033N, R1033G, R1033Y, K1044R, R1049S, R1049C, Q1047R, V1056A, N1064S, and L1075M, relative to the amino acid sequence of SEQ ID NO: 5.

In some embodiments, the amino acid sequence of the Cas protein comprises a substitution at amino acid position 6 of SEQ ID NO: 5, or a corresponding mutation in another Nme2Cas9 protein. In certain embodiments, the substitution is an P6S substitution.

In some embodiments, the amino acid sequence of the Cas protein comprises a substitution at amino acid position 33 of SEQ ID NO: 5, or a corresponding mutation in another Nme2Cas9 protein. In certain embodiments, the substitution is an E33G substitution.

In some embodiments, the amino acid sequence of the Cas protein comprises a substitution at amino acid position 47 of SEQ ID NO: 5, or a corresponding mutation in another Nme2Cas9 protein. In certain embodiments, the substitution is an E47K substitution.

In some embodiments, the amino acid sequence of the Cas protein comprises a substitution at amino acid position 63 of SEQ ID NO: 5, or a corresponding mutation in another Nme2Cas9 protein. In certain embodiments, the substitution is an R63K substitution.

In some embodiments, the amino acid sequence of the Cas protein comprises a substitution at amino acid position 68 of SEQ ID NO: 5, or a corresponding mutation in another Nme2Cas9 protein. In certain embodiments, the substitution is an V68M substitution.

In some embodiments, the amino acid sequence of the Cas protein comprises a substitution at amino acid position 104 of SEQ ID NO: 5, or a corresponding mutation in another Nme2Cas9 protein. In certain embodiments, the substitution is an K104T substitution.

In some embodiments, the amino acid sequence of the Cas protein comprises a substitution at amino acid position 116 of SEQ ID NO: 5, or a corresponding mutation in another Nme2Cas9 protein. In certain embodiments, the substitution is an A116T substitution.

In some embodiments, the amino acid sequence of the Cas protein comprises a substitution at amino acid position 123 of SEQ ID NO: 5, or a corresponding mutation in another Nme2Cas9 protein. In certain embodiments, the substitution is an T123A substitution.

In some embodiments, the amino acid sequence of the Cas protein comprises a substitution at amino acid position 152 of SEQ ID NO: 5, or a corresponding mutation in another Nme2Cas9 protein. In certain embodiments, the substitution is an D152A substitution.

In some embodiments, the amino acid sequence of the Cas protein comprises a substitution at amino acid position 152 of SEQ ID NO: 5, or a corresponding mutation in another Nme2Cas9 protein. In certain embodiments, the substitution is an D152N substitution.

In some embodiments, the amino acid sequence of the Cas protein comprises a substitution at amino acid position 152 of SEQ ID NO: 5, or a corresponding mutation in another Nme2Cas9 protein. In certain embodiments, the substitution is an D152G substitution.

In some embodiments, the amino acid sequence of the Cas protein comprises a substitution at amino acid position 154 of SEQ ID NO: 5, or a corresponding mutation in another Nme2Cas9 protein. In certain embodiments, the substitution is an E154K substitution.

In some embodiments, the amino acid sequence of the Cas protein comprises a substitution at amino acid position 221 of SEQ ID NO: 5, or a corresponding mutation in another Nme2Cas9 protein. In certain embodiments, the substitution is an E221D substitution.

In some embodiments, the amino acid sequence of the Cas protein comprises a substitution at amino acid position 260 of SEQ ID NO: 5, or a corresponding mutation in another Nme2Cas9 protein. In certain embodiments, the substitution is an F260L substitution.

In some embodiments, the amino acid sequence of the Cas protein comprises a substitution at amino acid position 263 of SEQ ID NO: 5, or a corresponding mutation in another Nme2Cas9 protein. In certain embodiments, the substitution is an A263T substitution.

In some embodiments, the amino acid sequence of the Cas protein comprises a substitution at amino acid position 303 of SEQ ID NO: 5, or a corresponding mutation in another Nme2Cas9 protein. In certain embodiments, the substitution is an A303S substitution.

In some embodiments, the amino acid sequence of the Cas protein comprises a substitution at amino acid position 396 of SEQ ID NO: 5, or a corresponding mutation in another Nme2Cas9 protein. In certain embodiments, the substitution is an T396A substitution.

In some embodiments, the amino acid sequence of the Cas protein comprises a substitution at amino acid position 413 of SEQ ID NO: 5, or a corresponding mutation in another Nme2Cas9 protein. In certain embodiments, the substitution is an H413N substitution.

In some embodiments, the amino acid sequence of the Cas protein comprises a substitution at amino acid position 427 of SEQ ID NO: 5, or a corresponding mutation in another Nme2Cas9 protein. In certain embodiments, the substitution is an A427S substitution.

In some embodiments, the amino acid sequence of the Cas protein comprises a substitution at amino acid position 451 of SEQ ID NO: 5, or a corresponding mutation in another Nme2Cas9 protein. In certain embodiments, the substitution is an D45IV substitution.

In some embodiments, the amino acid sequence of the Cas protein comprises a substitution at amino acid position 452 of SEQ ID NO: 5, or a corresponding mutation in another Nme2Cas9 protein. In certain embodiments, the substitution is an H452R substitution.

In some embodiments, the amino acid sequence of the Cas protein comprises a substitution at amino acid position 460 of SEQ ID NO: 5, or a corresponding mutation in another Nme2Cas9 protein. In certain embodiments, the substitution is an E460A substitution.

In some embodiments, the amino acid sequence of the Cas protein comprises a substitution at amino acid position 460 of SEQ ID NO: 5, or a corresponding mutation in another Nme2Cas9 protein. In certain embodiments, the substitution is an E460K substitution.

In some embodiments, the amino acid sequence of the Cas protein comprises a substitution at amino acid position 484 of SEQ ID NO: 5, or a corresponding mutation in another Nme2Cas9 protein. In certain embodiments, the substitution is an A484T substitution.

In some embodiments, the amino acid sequence of the Cas protein comprises a substitution at amino acid position 520 of SEQ ID NO: 5, or a corresponding mutation in another Nme2Cas9 protein. In certain embodiments, the substitution is an E520A substitution.

In some embodiments, the amino acid sequence of the Cas protein comprises a substitution at amino acid position 629 of SEQ ID NO: 5, or a corresponding mutation in another Nme2Cas9 protein. In certain embodiments, the substitution is an S629P substitution.

In some embodiments, the amino acid sequence of the Cas protein comprises a substitution at amino acid position 646 of SEQ ID NO: 5, or a corresponding mutation in another Nme2Cas9 protein. In certain embodiments, the substitution is an R646S substitution.

In some embodiments, the amino acid sequence of the Cas protein comprises a substitution at amino acid position 674 of SEQ ID NO: 5, or a corresponding mutation in another Nme2Cas9 protein. In certain embodiments, the substitution is an N674S substitution.

In some embodiments, the amino acid sequence of the Cas protein comprises a substitution at amino acid position 696 of SEQ ID NO: 5, or a corresponding mutation in another Nme2Cas9 protein. In certain embodiments, the substitution is an F696V substitution.

In some embodiments, the amino acid sequence of the Cas protein comprises a substitution at amino acid position 711 of SEQ ID NO: 5, or a corresponding mutation in another Nme2Cas9 protein. In certain embodiments, the substitution is an G711R substitution.

In some embodiments, the amino acid sequence of the Cas protein comprises a substitution at amino acid position 720 of SEQ ID NO: 5, or a corresponding mutation in another Nme2Cas9 protein. In certain embodiments, the substitution is an D720A substitution.

In some embodiments, the amino acid sequence of the Cas protein comprises a substitution at amino acid position 724 of SEQ ID NO: 5, or a corresponding mutation in another Nme2Cas9 protein. In certain embodiments, the substitution is an A724S substitution.

In some embodiments, the amino acid sequence of the Cas protein comprises a substitution at amino acid position 758 of SEQ ID NO: 5, or a corresponding mutation in another Nme2Cas9 protein. In certain embodiments, the substitution is an 1758V substitution.

In some embodiments, the amino acid sequence of the Cas protein comprises a substitution at amino acid position 765 of SEQ ID NO: 5, or a corresponding mutation in another Nme2Cas9 protein. In certain embodiments, the substitution is an V765A substitution.

In some embodiments, the amino acid sequence of the Cas protein comprises a substitution at amino acid position 767 of SEQ ID NO: 5, or a corresponding mutation in another Nme2Cas9 protein. In certain embodiments, the substitution is an H767Y substitution.

In some embodiments, the amino acid sequence of the Cas protein comprises a substitution at amino acid position 769 of SEQ ID NO: 5, or a corresponding mutation in another Nme2Cas9 protein. In certain embodiments, the substitution is an K769R substitution.

In some embodiments, the amino acid sequence of the Cas protein comprises a substitution at amino acid position 771 of SEQ ID NO: 5, or a corresponding mutation in another Nme2Cas9 protein. In certain embodiments, the substitution is an H771R substitution.

In some embodiments, the amino acid sequence of the Cas protein comprises a substitution at amino acid position 816 of SEQ ID NO: 5, or a corresponding mutation in another Nme2Cas9 protein. In certain embodiments, the substitution is an S816I substitution.

In some embodiments, the amino acid sequence of the Cas protein comprises a substitution at amino acid position 821 of SEQ ID NO: 5, or a corresponding mutation in another Nme2Cas9 protein. In certain embodiments, the substitution is an V821A substitution.

In some embodiments, the amino acid sequence of the Cas protein comprises a substitution at amino acid position 844 of SEQ ID NO: 5, or a corresponding mutation in another Nme2Cas9 protein. In certain embodiments, the substitution is an D844A substitution.

In some embodiments, the amino acid sequence of the Cas protein comprises a substitution at amino acid position 859 of SEQ ID NO: 5, or a corresponding mutation in another Nme2Cas9 protein. In certain embodiments, the substitution is an 1859V substitution.

In some embodiments, the amino acid sequence of the Cas protein comprises a substitution at amino acid position 865 of SEQ ID NO: 5, or a corresponding mutation in another Nme2Cas9 protein. In certain embodiments, the substitution is an W865L substitution.

In some embodiments, the amino acid sequence of the Cas protein comprises a substitution at amino acid position 932 of SEQ ID NO: 5, or a corresponding mutation in another Nme2Cas9 protein. In certain embodiments, the substitution is an E932K substitution.

In some embodiments, the amino acid sequence of the Cas protein comprises a substitution at amino acid position 940 of SEQ ID NO: 5, or a corresponding mutation in another Nme2Cas9 protein. In certain embodiments, the substitution is an K940R substitution.

In some embodiments, the amino acid sequence of the Cas protein comprises a substitution at amino acid position 951 of SEQ ID NO: 5, or a corresponding mutation in another Nme2Cas9 protein. In certain embodiments, the substitution is an M951R substitution.

In some embodiments, the amino acid sequence of the Cas protein comprises a substitution at amino acid position 1005 of SEQ ID NO: 5, or a corresponding mutation in another Nme2Cas9 protein. In certain embodiments, the substitution is an K1005R substitution.

In some embodiments, the amino acid sequence of the Cas protein comprises a substitution at amino acid position 1028 of SEQ ID NO: 5, or a corresponding mutation in another Nme2Cas9 protein. In certain embodiments, the substitution is an D1028N substitution.

In some embodiments, the amino acid sequence of the Cas protein comprises a substitution at amino acid position 1029 of SEQ ID NO: 5, or a corresponding mutation in another Nme2Cas9 protein. In certain embodiments, the substitution is an S1029A substitution.

In some embodiments, the amino acid sequence of the Cas protein comprises a substitution at amino acid position 1031 of SEQ ID NO: 5, or a corresponding mutation in another Nme2Cas9 protein. In certain embodiments, the substitution is an N1031S substitution.

In some embodiments, the amino acid sequence of the Cas protein comprises a substitution at amino acid position 1033 of SEQ ID NO: 5, or a corresponding mutation in another Nme2Cas9 protein. In certain embodiments, the substitution is an R1033N substitution.

In some embodiments, the amino acid sequence of the Cas protein comprises a substitution at amino acid position 1033 of SEQ ID NO: 5, or a corresponding mutation in another Nme2Cas9 protein. In certain embodiments, the substitution is an R10330 substitution.

In some embodiments, the amino acid sequence of the Cas protein comprises a substitution at amino acid position 1033 of SEQ ID NO: 5, or a corresponding mutation in another Nme2Cas9 protein. In certain embodiments, the substitution is an R1033Y substitution.

In some embodiments, the amino acid sequence of the Cas protein comprises a substitution at amino acid position 1044 of SEQ ID NO: 5, or a corresponding mutation in another Nme2Cas9 protein. In certain embodiments, the substitution is an K1044R substitution.

In some embodiments, the amino acid sequence of the Cas protein comprises a substitution at amino acid position 1049 of SEQ ID NO: 5, or a corresponding mutation in another Nme2Cas9 protein. In certain embodiments, the substitution is an R1049S substitution.

In some embodiments, the amino acid sequence of the Cas protein comprises a substitution at amino acid position 1049 of SEQ ID NO: 5, or a corresponding mutation in another Nme2Cas9 protein. In certain embodiments, the substitution is an R1049C substitution.

In some embodiments, the amino acid sequence of the Cas protein comprises a substitution at amino acid position 1047 of SEQ ID NO: 5, or a corresponding mutation in another Nme2Cas9 protein. In certain embodiments, the substitution is an Q1047R substitution.

In some embodiments, the amino acid sequence of the Cas protein comprises a substitution at amino acid position 1056 of SEQ ID NO: 5, or a corresponding mutation in another Nme2Cas9 protein. In certain embodiments, the substitution is an V1056A substitution.

In some embodiments, the amino acid sequence of the Cas protein comprises a substitution at amino acid position 1064 of SEQ ID NO: 5, or a corresponding mutation in another Nme2Cas9 protein. In certain embodiments, the substitution is an N1064S substitution.

In some embodiments, the amino acid sequence of the Cas protein comprises a substitution at amino acid position 1075 of SEQ ID NO: 5, or a corresponding mutation in another Nme2Cas9 protein. In certain embodiments, the substitution is an L1075M substitution.

It should be appreciated that any of the amino acid mutations described herein, (e.g., E47K) from a first amino acid residue (e.g., E) to a second amino acid residue (e.g., K) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., an A58T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine: asparagine and glutamine: methionine and cysteine; aspartic acid and glutamic acid; and arginine and lysine.

The skilled artisan would recognize that such conservative amino acid substitutions will likely have minor effects on protein structure and are likely to be well tolerated without compromising function. In some embodiments, any of the amino acid mutations provided herein from one amino acid to a threonine may be an amino acid mutation to a serine. In some embodiments, any of the amino acid mutations provided herein from one amino acid to an arginine may be an amino acid mutation to a lysine. In some embodiments, any of the amino acid mutations provided herein from one amino acid to an isoleucine may be an amino acid mutation to an alanine, valine, methionine, or leucine. In some embodiments, any of the amino acid mutations provided herein from one amino acid to a lysine may be an amino acid mutation to an arginine. In some embodiments, any of the amino acid mutations provided herein from one amino acid to an aspartic acid may be an amino acid mutation to a glutamic acid or asparagine. In some embodiments, any of the amino acid mutations provided herein from one amino acid to a valine may be an amino acid mutation to an alanine, isoleucine, methionine, or leucine. In some embodiments, any of the amino acid mutations provided herein from one amino acid to a glycine may be an amino acid mutation to an alanine. It should be appreciated, however, that additional conserved amino acid residues would be recognized by the skilled artisan, and any of the amino acid mutations to other conserved amino acid residues are also within the scope of this disclosure.

In some embodiments, the Cas variants of the disclosure are variants of Nme1Cas9 or Nme3Cas9, which share a high degree of homology with Nme2Cas9. The amino acid sequence of NmeCas9 (or Nme1Cas9) is provided below, as SEQ ID NO: 6. The length of this protein is 1083 amino acids.

(SEQ ID NO: 6)
MAAFKPNSINYILGLDIGIASVGWAMVEIDEEENPIRLIDLGVRV
FERAEVPKTGDSLAMARRLARSVRRLTRRRAHRLLRTRRLLKREG
VLQAANFDENGLIKSLPNTPWQLRAAALDRKLTPLEWSAVLLHLI
KHRGYLSQRKNEGETADKELGALLKGVAGNAHALQTGDFRTPAEL
ALNKFEKESGHIRNQRSDYSHTFSRKDLQAELILLFEKQKEFGNP
HVSGGLKEGIETLLMTQRPALSGDAVQKMLGHCTFEPAEPKAAKN
TYTAERFIWLTKLNNLRILEQGSERPLTDTERATLMDEPYRKSKL
TYAQARKLLGLEDTAFFKGLRYGKDNAEASTLMEMKAYHAISRAL
EKEGLKDKKSPLNLSPELQDEIGTAFSLFKTDEDITGRLKDRIQP
EILEALLKHISFDKFVQISLKALRRIVPLMEQGKRYDEACAEIYG
DHYGKKNTEEKIYLPPIPADEIRNPVVLRALSQARKVINGVVRRY
GSPARIHIETAREVGKSFKDRKEIEKRQEENRKDREKAAAKFREY
FPNFVGEPKSKDILKLRLYEQQHGKCLYSGKEINLGRLNEKGYVE
IDAALPFSRTWDDSFNNKVLVLGSENQNKGNQTPYEYFNGKDNSR
EWQEFKARVETSRFPRSKKQRILLQKFDEDGFKERNLNDTRYVNR
FLCQFVADRMRLTGKGKKRVFASNGQITNLLRGFWGLRKVRAEND
RHHALDAVVVACSTVAMQQKITRFVRYKEMNAFDGKTIDKETGEV
LHQKTHFPQPWEFFAQEVMIRVFGKPDGKPEFEEADTLEKLRTLL
AEKLSSRPEAVHEYVTPLFVSRAPNRKMSGQGHMETVKSAKRLDE
GVSVLRVPLTQLKLKDLEKMVNREREPKLYEALKARLEAHKDDPA
KAFAEPFYKYDKAGNRTQQVKAVRVEQVQKTGVWVRNHNGIADNA
TMVRVDVFEKGDKYYLVPIYSWQVAKGILPDRAVVQGKDEEDWQL
IDDSFNFKFSLHPNDLVEVITKKARMFGYFASCHRGTGNINIRIH
DLDHKIGKNGILEGIGVKTALSFQKYQIDELGKEIRPCRLKKRPP
VR

In some embodiments, the Cas protein of the disclosure is a Cas variant that exhibits increased activity on a target sequence as compared to a wild-type Cas9 protein. In some embodiments the Cas9 protein is an Nme2Ca9 variant compared to wild-type Nme2Cas9 protein. In some embodiments the Nme2Cas9 variant is any one of SEQ ID NOs: 1-4 compared to wild-type Nme2Cas9 protein of SEQ ID NO: 5. In some embodiments, the Cas protein exhibits an activity on a target sequence that is increased by at least 2-fold, at least 3-fold, at least 4-fold, at least 5-fold, at least 6-fold, at least 7-fold, at least 8-fold, at least 9-fold, or at least 10-fold as compared to a wild-type Nme2Cas9 protein as provided by SEQ ID NO: 5.

In some aspects, the present disclosure provides fusion proteins comprising any of the Nme2Cas9 variants provided herein. In some embodiments, the fusion proteins comprise (i) any of the Nme2Cas9 variants provided herein, and (ii) an effector domain. In some embodiments, the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity. In certain embodiments, the effector domain is a nucleic acid editing domain (e.g., a deaminase domain). A fusion protein comprising a Cas protein and a deaminase domain may be referred to herein as a “base editor.” In some embodiments, the deaminase domain is an adenosine deaminase domain (e.g., an E. coli Tad A (ecTadA) deaminase domain) or a cytidine deaminase domain (e.g., an APOBEC family deaminase domain). In some embodiments, a base editor fusion protein comprising any of the Cas variants provided herein exhibits increased base editing activity on a target sequence as compared to a fusion protein comprising a wild-type Nme2Cas9 protein as provided by SEQ ID NO: 5. In certain embodiments, the activity is increased by at least 2-fold, at least 3-fold, at least 4-fold, at least 5-fold, at least 6-fold, at least 7-fold, at least 8-fold, at least 9-fold, or at least 10-fold as compared to a wild-type Nme2Cas9 protein.

In some aspects, the present disclosure provides Cas variants comprising substitutions corresponding to any of the substitutions disclosed herein, or any combination thereof, in another Cas protein homolog. The amino acid substitutions disclosed herein are compatible with a variety of Cas homologs known in the art. The amino acid substitutions disclosed herein are broadly compatible with and may be made at corresponding positions in a variety of napDNAbps that include, but are not limited to, Cas9 proteins, Cas12 and Cas14 proteins. Additional Cas9 and Cas12 variants and homologs include Cas9 (e.g., dCas9 and nCas9), Cpf1, CjCas9, SauriCas9, SpRY, SpRY-HF1, CasX, CasY, C2c1, C2c2, C2c3, GeoCas9, CjCas9, Cas12a, Cas12b, Cas12g, Cas12h, Cas12i, Cas13b, Cas13c, Cas13d, Cas14, Cas14a1, Csn2, xCas9, SpCas9-NG, Argonaute (Ago), Cas9-KKH, SmacCas9, Spy-macCas9, SpCas9-VRQR, SpCas9-NRRH, SpaCas9-NRTH, SpCas9-NRCH, LbCas12a, AsCas12a, CeCas12a, MbCas12a, Cas3, CasO, and circularly permuted Cas9 proteins such as CP1012, CP1028, CP1041, CP1249, and CP1300, and variants and homologs thereof. Exemplary Cas14 homologs include, but are not limited to, Cas14 proteins, including Cas14a1, Cas14a2, Cas14a3, Cas14a4, Cas14a5, Cas14a6. Cas14b1, Cas14b2, Cas14b3, Cas14b4, Cas14b5, Cas14b6, Cas14b7, Cas14b8, Cas14b9, Cas14b10, Cas14b11, Cas14b12, Cas14b13, Cas14b14, Cas14b15, Cas14b16, Cas14c1, Cas14c2, Cas14d1. Cas14d2, Cas14d3. Cas14e1, Cas14e2, Cas14e3, Cas14f1. Cas14f2, Cas14g, Cas14g2, Cas14h1, Cas14h2, Cas14h3, Cas14u1, Cas14u2, Cas14u3, Cas14u4, Cas14u5, Cas14u6, Cas14u7, and Cas14u8. The amino acid substitutions disclosed herein may be made at corresponding positions in any Cas protein or other napDNAbp, homolog thereof, or variant thereof known in the art, and the present disclosure is not limited in this respect.

The following table provides a comparison of the PAM preferences targeted by the presently disclosed Nme2Cas variants to those targeted by other Cas homologs (R=any purine, Y=any pyrimidine, N=any nucleotide).

TABLE 6
PAM preferences of Exemplary Cas homologs

| Cas homolog | PAM preference(s) |
|---|---|
| S. pyogenes Cas9 (SpCas9) | NGG [canonical] |
| WT Nme1Cas9 | N₄GAYW/N₄GYTT/N₄GTCT |
| WT Nme2Cas9 | NNNNCC or NNNNGATT |
| eNme2-C, eNme2-C.NR | NNNNCN |
| eNme2-T.1, eNme2-T.2 | NNNNTN |
| eNme2-N1-21 | NNNTTG |
| SaCas9 | NNGRRT/NGRRT or NNGRRN/NGRRN |
| SaCas9-KKH | NNNRRT or NNGRRT |
| SauriCas9 | NNGG |
| CjCas9 | NNNNACA, NNNNRYAC, or |
| | NNNNACAC |
| StCas9 | NNAGAAW |
| SpRY | NYN |
| SmacCas9 | NAA/TAAA |
| SpCas9-VRQR | NGAN or NGNG |
| SpCas9-EQR | NGAG |
| SpCas9-VRER | NGCG |

### Base Editing and Deaminase Domains

In some embodiments, the fusion proteins described herein are base editor fusion proteins. Base editors may comprise a deaminase domain (e.g., when the Cas proteins provided herein are being used in the context of a base editor). A deaminase domain may be a cytidine deaminase domain or an adenosine deaminase domain. In some embodiments, the fusion protein comprises at least 1 (e.g., at least 1, at least 2, at least 3, at least 4) nuclear localization sequence (NLS). In some embodiments, the fusion protein comprises a first NLS. In some embodiments, the fusions protein comprises a second NLS. In some embodiments, the fusion protein comprises a first and a second NLS. In some embodiments, the NLS are the same NLS amino acid sequence. In some embodiments, the NLS are different NLS amino acid sequences.

In some aspects of the present disclosure, the base editor construct encodes a cytosine base editor or an adenine base editor. In some embodiments, the base editor construct encodes an adenine base editor. In some embodiments, the base editor construct encodes a cytosine base editor. Base editors that convert a cytidine (C) to a thymidine (T) are cytosine base editors (CBEs). CBEs comprise a cytidine deaminase domain that catalyzes the conversion of a C to a T. A “cytidine deaminase” refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T base editor comprises a Nme2Cas9 variant provided herein fused to a cytidine deaminase. In some embodiments, the cytidine deaminase domain is fused to the N-terminus of the Nme2Cas9 variant.

The cytidine deaminase domains of the disclosed cytosine base editors may comprise variants of wild-type cytidine deaminases. These variants may comprise an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type deaminase. In some embodiments, any of the cytidine deaminase domains may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of the wild type enzyme. These differences may comprise nucleotides that have been inserted, deleted, or substituted relative to the amino acid sequence of the wild type enzyme. In some embodiments, the disclosed cytidine deaminase domains contain stretches of about 50, about 75, about 100, about 125, about 150, about 175, about 200, about 300, about 400, about 500, or more than 500 consecutive amino acids in common with the wild type enzyme. In some embodiments, the cytidine deaminase domains comprise truncations at the N-terminus or C-terminus relative to the wild-type enzyme. In some embodiments, the disclosed cytosine base editors may comprise modified cytidine deaminases (e.g., YE 1, R33A, or R33A+K34A).

The cytosine base editors (CBEs) of the disclosure may further comprise one or more nuclear localization signals (NLSs) and/or two or more uracil glycosylase inhibitor (UGI) domains. In some embodiments, the disclosed CBEs comprise two UGI domains (i.e., a first UGI domain and a second UGI domain). Thus, the base editors may comprise the structure: NH2-[first nuclear localization sequence]-[cytidine deaminase domain]-[napDNAbp domain]-[first UGI domain]-[second UGI domain]-[second nuclear localization sequence]-COOH, wherein each instance of “]-[” indicates the presence of an optional linker sequence. Exemplary CBEs may have a structure that comprises the “BE4max” architecture, with an NH₂-[NLS]-[APOBEC1 deaminase]-[Cas9 nickase]-[UGI domain]-[UGI domain]-[NLS]-COOH structure, having optimized nuclear localization signals, the cytidine deaminase is a rat APOBEC1 (rAPOBEC1) deaminase (SEQ ID NO: 51) and wherein the napDNAbp domain comprises an eNme2Cas9 variant. This BE4max structure was reported to have optimized codon usage for expression in human cells, as reported in Koblan et al., Nat Biotechnol. 2018; 36(9):843-846, incorporated herein by reference. Additional exemplary CBEs are disclosed in PCT Publication No. WO 2019/023680, published Jan. 31, 2019, and PCT Publication No. WO 2021/108717, published Jun. 3, 2021, each of which are incorporated herein by reference.

Accordingly, the fusion proteins of the present disclosure may comprise CBEs comprising a napDNAbp domain (e.g., any of the Nme2Cas9 variants provided herein) and a cytidine deaminase domain that enzymatically deaminates a cytosine nucleobase of a C:G nucleobase pair to a uracil. The uracil may be subsequently converted to a thymine (T) by the cell's DNA repair and replication machinery. The mismatched guanine (G) on the opposite strand may subsequently be converted to an adenine (A) by the cell's DNA repair and replication machinery. In this manner, a target C:G nucleobase pair is ultimately converted to a T:A nucleobase pair. Other cytidine deaminase domains besides those provided herein are known in the art, and a person of ordinary skill in the art would recognize which cytidine deaminase domains could be used in the fusion proteins of the present disclosure. The CBE fusion proteins of the present disclosure may comprise modified (or evolved) cytidine deaminase domains, such as deaminase domains that recognize an expanded PAM sequence, have improved efficiency of deaminating 5′-GC targets, and/or make edits in a narrower target window.

Non-limiting examples of suitable cytidine deaminase domains of the disclosed CBEs are provided below, as SEQ ID NOs: 33-56 and 177-186:

Human AID
(SEQ ID NO: 33)
MDSLLMNRRKFLYQFKNVRWAKGRRETYLCYVVKRRDSATSFSLD
FGYLRNKNGCHVELLFLRYISDWDLDPGRCYRVTWFTSWSPCYDC
ARHVADFLRGNPNLSLRIFTARLYFCEDRKAEPEGLRRLHRAGVQ
IAIMTFKDYFYCWNTFVENHERTFKAWEGLHENSVRLSRQLRRIL
LPLYEVDDLRDAFRTLGL
Mouse AID
(SEQ ID NO: 34)
MDSLLMKQKKFLYHFKNVRWAKGRHETYLCYVVKRRDSATSCSLD
FGHLRNKSGCHVELLFLRYISDWDLDPGRCYRVTWFTSWSPCYDC
ARHVAEFLRWNPNLSLRIFTARLYFCEDRKAEPEGLRRLHRAGVQ
IGIMTFKDYFYCWNTFVENRERTFKAWEGLHENSVRLTRQLRRIL
LPLYEVDDLRDAFRMLGF
Dog AID
(SEQ ID NO: 35)
MDSLLMKQRKFLYHFKNVRWAKGRHETYLCYVVKRRDSATSFSLD
FGHLRNKSGCHVELLFLRYISDWDLDPGRCYRVTWFTSWSPCYDC
ARHVADFLRGYPNLSLRIFAARLYFCEDRKAEPEGLRRLHRAGVQ
IAIMTFKDYFYCWNTFVENREKTFKAWEGLHENSVRLSRQLRRIL
LPLYEVDDLRDAFRTLGL
Bovine AID
(SEQ ID NO: 36)
MDSLLKKQRQFLYQFKNVRWAKGRHETYLCYVVKRRDSPTSFSLD
FGHLRNKAGCHVELLFLRYISDWDLDPGRCYRVTWFTSWSPCYDC
ARHVADFLRGYPNLSLRIFTARLYFCDKERKAEPEGLRRLHRAGV
QIAIMTFKDYFYCWNTFVENHERTFKAWEGLHENSVRLSRQLRRI
LLPLYEVDDLRDAFRTLGL
Mouse APOBEC-3
(SEQ ID NO: 37)
MGPFCLGCSHRKCYSPIRNLISQETFKFHFKNLGYAKGRKDTFLC
YEVTRKDCDSPVSLHHGVFKNKDNIHAEICFLYWFHDKVLKVLSP
REEFKITWYMSWSPCFECAEQIVRFLATHHNLSLDIFSSRLYNVQ
DPETQQNLCRLVQEGAQVAAMDLYEFKKCWKKFVDNGGRRFRPWK
RLLTNFRYQDSKLQEILRPCYIPVPSSSSSTLSNICLTKGLPETR
FCVEGRRMDPLSBEEFYSQFYNQRVKHLCYYHRMKPYLCYQLEQF
NGQAPLKGCLLSEKGKQHAEILFLDKIRSMELSQVTITCYLTWSP
CPNCAWQLAAFKRDRPDLILHIYTSRLYFHWKRPFQKGLCSLWQS
GILVDVMDLPQFTDCWTNFVNPKRPFWPWKGLEIISRRTQRRLRR
IKESWGLQDLVNDFGNLQLGPPMS
Rat APOBEC-3
(SEQ ID NO: 38)
MGPFCLGCSHRKCYSPIRNLISQETFKFHFKNLRYAIDRKDTFLC
YEVTRKDCDSPVSLHHGVFKNKDNIHAEICFLYWFHDKVLKVLSP
REEFKITWYMSWSPCFECAEQVLRFLATHHNLSLDIFSSRLYNIR
DPENQQNLCRLVQEGAQVAAMDLYEFKKCWKKFVDNGGRRFRPWK
KLLTNFRYQDSKLQEILRPCYIPVPSSSSSTLSNICLTKGLPETR
FCVERRRVHLLSEEEFYSQFYNQRVKHLCYYHGVKPYLCYQLEQF
NGQAPLKGCLLSEKGKQHAEILFLDKIRSMELSQVIITCYLTWSP
CPNCAWQLAAFKRDRPDLILHIYTSRLYFHWKRPFQKGLCSLWQS
GILVDVMDLPQFTDCWTNFVNPKRPFWPWKGLEIISRRTQRRLHR
IKESWGLQDLVNDFGNLQLGPPMS
Rhesus macaque APOBEC-3G
(SEQ ID NO: 39)
MVEPMDPRTFVSNENNRPILSGLNTVWLCCEVKTKDPSGPPLDAK
IFQGKVYSKAKYHPEMRFLRWFHKWRQLHHDQEYKVTWYVSWSPC
TRCANSVATFLAKDPKVTLTIFVARLYYFWKPDYQQALRILCQKR
GGPHATMKIMNYNEFQDCWNKFVDGRGKPFKPRNNLPKHYTLLQA
TLGELLRHLMDPGTFTSNENNKPWVSGQHETYLCYKVERLHNDTW
VPLNQHRGFLRNQAPNIHGFPKGRHAELCFLDLIPFWKLDGQQYR
VTCFTSWSPCFSCAQEMAKFISNNEHVSLCIFAARIYDDQGRYQE
GLRALHRDGAKIAMMNYSEFEYCWDTFVDRQGRPFQPWDGLDEHS
QALSGRLRAI
Chimpanzee APOBEC-3G
(SEQ ID NO: 40)
MKPHFRNPVERMYQDTFSDNFYNRPILSHRNTVWLCYEVKTKGPS
RPPLDAKIFRGQVYSKLKYHPEMRFFHWFSKWRKLHRDQEYEVTW
YISWSPCTKCTRDVATFLAEDPKVTLTIFVARLYYFWDPDYQEAL
RSLCQKRDGPRATMKIMNYDEFQHCWSKFVYSQRELFEPWNNLPK
YYILLHIMLGEILRHSMDPPTFTSNENNELWVRGRHETYLCYEVE
RLHNDTWVLLNQRRGFLCNQAPHKHGFLEGRHAELCFLDVIPFWK
LDLHQDYRVTCFTSWSPCFSCAQEMAKFISNNKHVSLCIFAARIY
DDQGRCQEGLRTLAKAGAKISIMTYSEFKHCWDTFVDHQGCPFQP
WDGLEEHSQALSGRLRAILQNQGN
Green monkey APOBEC-3G
(SEQ ID NO: 41)
MNPQIRNMVEQMEPDIFVYYFNNRPILSGRNTVWLCYEVKTKDPS
GPPLDANIFQGKLYPEAKDHPEMKFLHWFRKWRQLHRDQEYEVTW
YVSWSPCTRCANSVATFLAEDPKVTLTIFVARLYYFWKPDYQQAL
RILCQERGGPHATMKIMNYNEFQHCWNEFVDGQGKPFKPRKNLPK
HYTLLHATLGELLRHVMDPGTFTSNFNNKPWVSGQRETYLCYKVE
RSHNDTWVLLNQHRGFLRNQAPDRHGFPKGRHAELCFLDLIPFWK
LDDQQYRVTCFTSWSPCFSCAQKMAKFISNNKHVSLCIFAARIYD
DQGRCQEGLRTLHRDGAKIAVMNYSEFEYCWDTFVDRQGRPFQPW
DGLDEHSQALSGRLRAI
Human APOBEC-3G
(SEQ ID NO: 42)
MKPHFRNTVERMYRDTFSYNFYNRPILSRRNTVWLCYEVKTKGPS
RPPLDAKIFRGQVYSELKYHPEMRFFHWFSKWRKLHRDQEYEVTW
YISWSPCTKCTRDMATFLAEDPKVTLTIFVARLYYFWDPDYQEAL
RSLCQKRDGPRATMKIMNYDEFQHCWSKFVYSQRELFEPWNNLPK
YYILLHIMLGEILRHSMDPPTFTFNFNNEPWVRGRHETYLCYEVE
RMHNDTWVLLNQRRGFLCNQAPHKHGFLEGRHAELCFLDVIPFWK
LDLDQDYRVTCFTSWSPCFSCAQEMAKFISKNKHVSLCIFTARIY
DDQGRCQEGLRTLAEAGAKISIMTYSEFKHCWDTFVDHQGCPFQP
WDGLDEHSQDLSGRLRAILQNQEN
Human APOBEC-3F
(SEQ ID NO: 43)
MKPHFRNTVERMYRDTFSYNFYNRPILSRRNTVWLCYEVKTKGPS
RPRLDAKIFRGQVYSQPEHHAEMCFLSWFCGNQLPAYKCFQITWF
VSWTPCPDCVAKLABFLAEHPNVTLTISAARLYYYWERDYRRALC
RLSQAGARVKIMDDEEFAYCWENFVYSEGQPFMPWYKFDDNYAFL
HRTLKEILRNPMEAMYPHIFYFHFKNLRKAYGRNESWLCFTMEVV
KHHSPVSWKRGVFRNQVDPETHCHAERCFLSWFCDDILSPNTNYE
VTWYTSWSPCPECAGEVAEFLARHSNVNLTIFTARLYYFWDTDYQ
EGLRSLSQEGASVEIMGYKDFKYCWENFVYNDDEPFKPWKGLKYN
FLFLDSKLQEILE
Human APOBEC-3B
(SEQ ID NO: 44)
MNPQIRNPMERMYRDTFYDNFENEPILYGRSYTWLCYEVKIKRGR
SNLLWDTGVFRGQVYFKPQYHAEMCFLSWFCGNQLPAYKCFQITW
FVSWTPCPDCVAKLAEFLSEHPNVTLTISAARLYYYWERDYRRAL
CRLSQAGARVTIMDYEEFAYCWENFVYNEGQQFMPWYKFDENYAF
LHRTLKEILRYLMDPDTFTFNFNNDPLVLRRRQTYLCYEVERLDN
GTWVLMDQHMGFLCNEAKNLLCGFYGRHAELRFLDLVPSLQLDPA
QIYRVTWFISWSPCFSWGCAGEVRAFLQENTHVRLRIFAARIYDY
DPLYKEALQMLRDAGAQVSIMTYDEFEYCWDTFVYRQGCPFQPWD
GLEEHSQALSGRLRAILQNQGN
Human APOBEC-3C
(SEQ ID NO: 45)
MNPQIRNPMKAMYPGTFYFQFKNLWEANDRNETWLCFTVEGIKRR
SVVSWKTGVFRNQVDSETHCHAERCFLSWFCDDILSPNTKYQVTW
YTSWSPCPDCAGEVAEFLARHSNVNLTIFTARLYYFQYPCYQEGL
RSLSQEGVAVEIMDYEDFKYCWENFVYNDNEPFKPWKGLKTNFRL
LKRRLRESLQ
Human APOBEC-3A
(SEQ ID NO: 46)
MEASPASGPRHLMDPHIFTSNFNNGIGRHKTYLCYEVERLDNGTS
VKMDQHRGFLHNQAKNLLCGFYGRHAELRFLDLVPSLQLDPAQIY
RVTWFISWSPCFSWGCAGEVRAFLQENTHVRLRIFAARIYDYDPL
YKEALQMLRDAGAQVSIMTYDEFKHCWDTFVDHQGCPFQPWDGLD
EHSQALSGRLRAILQNQGN
Human APOBEC-3H
(SEQ ID NO: 47)
MALLTAETFRLQFNNKRRLRRPYYPRKALLCYQLTPQNGSTPTRG
YFENKKKCHAEICFINEIKSMGLDETQCYQVTCYLTWSPCSSCAW
ELVDFIKAHDHLNLGIFASRLYYHWCKPQQKGLRLLCGSQVPVEV
MGFPKFADCWENFVDHEKPLSFNPYKMLEELDKNSRAIKRRLERI
KIPGVRAQGRYMDILCDAEV
Human APOBEC-3D
(SEQ ID NO: 48)
MNPQIRNPMERMYRDTFYDNFENEPILYGRSYTWLCYEVKIKRGR
SNLLWDTGVFRGPVLPKRQSNHRQEVYFRFENHAEMCFLSWFCGN
RLPANRRFQITWFVSWNPCLPCVVKVTKFLAEHPNVTLTISAARL
YYYRDRDWRWVLLRLHKAGARVKIMDYEDFAYCWENFVCNEGQPF
MPWYKFDDNYASLHRTLKEILRNPMEAMYPHIFYFHFKNLLKACG
RNESWLCFTMEVTKHHSAVFRKRGVFRNQVDPETHCHAERCFLSW
FCDDILSPNTNYEVTWYTSWSPCPECAGEVAEFLARHSNVNLTIF
TARLCYFWDTDYQEGLCSLSQEGASVKIMGYKDFVSCWKNFVYSD
DEPFKPWKGLQTNFRLLKRRLREILQ
Human APOBEC-1
(SEQ ID NO: 49)
MTSEKGPSTGDPTLRRRIEPWEFDVFYDPRELRKEACLLYEIKWG
MSRKIWRSSGKNTTNHVEVNFIKKFTSERDFHPSMSCSITWFLSW
SPCWECSQAIREFLSRHPGVTLVIYVARLFWHMDQQNRQGLRDLV
NSGVTIQIMRASEYYHCWRNFVNYPPGDEAHWPQYPPLWMMLYAL
ELHCIILSLPPCLKISRRWQNHLTFFRLHLQNCHYQTIPPHILLA
TGLIHPSVAWR
Mouse APOBEC-1
(SEQ ID NO: 50)
MSSETGPVAVDPTLRRRIEPHEFEVFFDPRELRKETCLLYEINWG
GRHSVWRHTSQNTSNHVEVNFLEKFTTERYFRPNTRCSITWFLSW
SPCGECSRAITEFLSRHPYVTLFIYIARLYHHTDQRNRQGLRDLI
SSGVTIQIMTEQEYCYCWRNFVNYPPSNEAYWPRYPHLWVKLYVL
ELYCIILGLPPCLKILRRKQPQLTFFTITLQTCHYQRIPPHLLWA
TGLK
Rat APOBEC-1 (rAPOBEC1)
(SEQ ID NO: 51)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
Petromyzon marinus CDA1 (pmCDA1)
(SEQ ID NO: 52)
MTDAEYVRIHEKLDIYTFKKQFFNNKKSVSHRCYVLFELKRRGER
RACFWGYAVNKPQSGTERGIHAEIFSIRKVEEYLRDNPGQFTINW
YSSWSPCADCAEKILEWYNQELRGNGHTLKIWACKLYYEKNARNQ
IGLWNLRDNGVGLNVMVSEHYQCCRKIFIQSSHNQLNENRWLEKT
LKRAEKRRSELSIMIQVKILHTTKSPAV
Evolved pmCDA1 (evoCDA1)
(SEQ ID NO: 53)
MTDABYVRIHEKLDIYTFKKQFSNNKKSVSHRCYVLFELKRRGER
RACFWGYAVNKPQSGTERGIHAEIFSIRKVEEYLRDNPGQFTINW
YSSWSPCADCAEKILEWYNQELRGNGHTLKIWVCKLYYEKNARNQ
IGLWNLRDNGVGLNVMVSEHYQCCRKIFIQSSHNQLNENRWLEKT
LKRAEKRRSELSIMFQVKILHTTKSPAV
Human APOBEC3G D316R_D317R
(SEQ ID NO: 54)
MKPHFRNTVERMYRDTFSYNFYNRPILSRRNTVWLCYEVKTKGPS
RPPLDAKIFRGQVYSELKYHPEMRFFHWFSKWRKLHRDQEYEVTW
YISWSPCTKCTRDMATFLAEDPKVTLTIFVARLYYFWDPDYQEAL
RSLCQKRDGPRATMKIMNYDEFQHCWSKFVYSQRELFEPWNNLPK
YYILLHIMLGEILRHSMDPPTFTENFNNEPWVRGRHETYLCYEVE
RMHNDTWVLLNQRRGFLCNQAPHKHGFLEGRHAELCFLDVIPFWK
LDLDQDYRVTCFTSWSPCFSCAQEMAKFISKNKHVSLCIFTARIY
RRQGRCQEGLRTLAEAGAKISIMTYSEFKHCWDTFVDHQGCPFQP
WDGLDEHSQDLSGRLRAILQNQEN
Human APOBEC3G chain A
(SEQ ID NO: 55)
MDPPTFTFNFNNEPWVRGRHETYLCYEVERMHNDTWVLLNQRRGF
LCNQAPHKHGFLEGRHAELCFLDVIPFWKLDLDQDYRVTCFTSWS
PCFSCAQEMAKFISKNKHVSLCIFTARIYDDQGRCQEGLRTLAEA
GAKISIMTYSEFKHCWDTFVDHQGCPFQPWDGLDEHSQDLSGRLR
AILQ
Human APOBEC3G chain A D120R_D121R
(SEQ ID NO: 56)
MDPPTFTFNFNNEPWVRGRHETYLCYEVERMHNDTWVLLNQRRGF
LCNQAPHKHGFLEGRHAELCFLDVIPFWKLDLDQDYRVTCFTSWS
PCFSCAQEMAKFISKNKHVSLCIFTARIYRRQGRCQEGLRTLAEA
GAKISIMTYSEFKHCWDTFVDHQGCPFQPWDGLDEHSQDLSGRLR
AILQ
evo APOBEC1
(SEQ ID NO: 177)
MSSKTGPVAVDPTLRRRIEPHEFEVFEDPRELRKETCLLYEINWG
GRHSIWRHTSQNTNKHVEVNFIEKFTTERYFCPNTRCSITWFLSW
SPCGECSRAITEFLSRYPNVTLFIYIARLYHLANPRNRQGLRDLI
SSGVTIQIMTEQESGYCWHNFVNYSPSNESHWPRYPHLWVRLYVL
ELYCIILGLPPCLNILRRKQSQLTSFTIALQSCHYQRLPPHILWA
TGLK
YE1
(SEQ ID NO: 178)
MSSETGPVAVDPTLRRRIEPHEFEVFFDPRELRKETCLLYEINWG
GRHSIWRHTSQNTNKHVEVNFIEKFTTERYFCPNTRCSITWFLSY
SPCGECSRAITEFLSRYPHVTLFIYIARLYHHADPENRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
YE2
(SEQ ID NO: 179)
MSSETGPVAVDPTLRRRIEPHEFEVFFDPRELRKETCLLYEINWG
GRHSIWRHTSQNTNKHVEVNFIEKFTTERYFCPNTRCSITWFLSY
SPCGECSRAITEFLSRYPHVTLFIYIARLYHHADPRNRQGLEDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
YEE
(SEQ ID NO: 180)
MSSETGPVAVDPTLRRRIEPHEFEVFFDPRELRKETCLLYEINWG
GRHSIWRHTSQNTNKHVEVNFIEKFTTERYFCPNTRCSITWFLSY
SPCGECSRAITEFLSRYPHVTLFIYIARLYHHADPENRQGLEDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
BE
(SEQ ID NO: 181)
MSSETGPVAVDPTLRRRIEPHEFEVFFDPRELRKETCLLYEINWG
GRHSIWRHTSQNTNKHVEVNFIEKFTTERYFCPNTRCSITWFLSW
SPCGECSRAITEFLSRYPHVTLFIYIARLYHHADPENRQGLEDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
R33A
(SEQ ID NO: 182)
MSSETGPVAVDPTLRRRIEPHEFEVFFDPRELAKETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
R33A + K34A
(SEQ ID NO: 183)
MSSETGPVAVDPTLRRRIEPHEFEVFFDPRELAAETCLLYEINWG
GRHSIWRHTSQNTNKHVEVNFIEKFTTERYFCPNTRCSITWFLSW
SPCGECSRAITEFLSRYPHVTLFIYIARLYHHADPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
AALN
(SEQ ID NO: 184)
MSSETGPVAVDPTLRRRIEPHEFEVFFDPRELAAETCLLYEINWG
GRHSIWRHTSQNTNKHVEVNFIEKFTTERYFCPNTRCSITWFLSW
SPCGECSRAITEFLSRYPHVTLFIYIARLYHLANPRNRQGLRDLI
SSGVTIQIMTEQESGYCWRNFVNYSPSNEAHWPRYPHLWVRLYVL
ELYCIILGLPPCLNILRRKQPQLTFFTIALQSCHYQRLPPHILWA
TGLK
FERNY
(SEQ ID NO: 185)
MFERNYDPRELRKETYLLYEIKWGKSGKLWRHWCQNNRTQHAEVY
FLENIFNARRFNPSTHCSITWYLSWSPCAECSQKIVDFLKEHPNV
NLEIYVARLYYHEDERNRQGLRDLVNSGVTIRIMDLPDYNYCWKT
FVSDQGGDEDYWPGHFAPWIKQYSLKL
evo FERNY
(SEQ ID NO: 186)
MFERNYDPRELRKETYLLYEIKWGKSGKLWRHWCQNNRTQHAEVY
FLENIFNARRFNPSTHCSITWYLSWSPCAECSQKIVDFLKEHPNV
NLEIYVARLYYPENERNRQGLRDLVNSGVTIRIMDLPDYNYCWKT
FVSDQGGDEDYWPGHFAPWIKQYSLKL

In some aspects, adenine base editors are provided. Base editors that convert an adenosine (A) to a guanosine (G) are adenine base editors (ABEs). ABEs comprise an adenosine deaminase domain that catalyzes the conversion of a A to a G. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no wild-type adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine for use in adenosine nucleobase editors have been described, e.g., in PCT Application PCT/US2017/045381, filed Aug. 3, 2017, which published as WO 2018/027078, PCT Application No. PCT/US2019/033848, which published as WO 2019/226953, PCT Application No. PCT/US2019/033848, filed May 23, 2019: PCT Publication No. WO 2020/214842, published Oct. 22, 2020: PCT Publication No. WO 2021/158921, published Aug. 12, 2021: Gaudelli et al., Nat Biotechnol. 2020 July; 38(7):892-900 and PCT Publication No. WO 2021/050571, published Mar. 18, 2021, each of which is incorporated herein by reference.

In some embodiments, the disclosed adenosine deaminases are variants of known adenosine deaminase TadA7.10, which comprises the following mutations as compared to wild-type ecTadA (SEQ 1D NO: 57): W23R, H36L, P48A, R51L, L84F, A106V, D108N, H123Y, S146C, D147Y, R152P, E155V, 1156F, and K157N. In some embodiments, the disclosed adenosine deaminases are variants of a TadA derived from a species other than E. coli, such as Staphylococcus aureus, Salmonella typhi, Shewanella putrefaciens, Haemophilus influenzae, Caulobacter crescentus, or Bacillus subtilis. In various embodiments, the disclosed adenosine deaminases hydrolytically deaminate a targeted adenosine in a nucleic acid of interest to an inosine, which is read as a guanosine by DNA polymerase enzymes.

In some embodiments, any of the disclosed adenine base editors are capable of deaminating adenosine in a nucleic acid sequence (e.g., DNA or RNA). The state-of-the-art ABE is ABE7.10, which is disclosed in International Publication No. WO 2018/027078, published Aug. 2, 2018. A more recently generated ABE is ABE8e, which contains an adenosine deaminase domain containing a single deaminase variant, TadA8e, as described in International Publication No. WO 2021/158921, published Aug. 12, 2021. TadA8e contains nine mutations relative to TadA7.10, the adenosine deaminase of ABE7.10. TadA7.10 is also the deaminase domain of ABEmax, which is a variant of ABE7.10 that has been codon optimized for expression in human cells. More recently, context-specific and context-preferential TadA deaminase variants Tad1, Tad6, and Tad6-SR were disclosed in International Application No. PCT/US2022/073781, filed Jul. 15, 2022, which is incorporated herein by reference.

The adenine base editors of the disclosure may further comprise one or more nuclear localization signals (NLSs). In exemplary embodiments, the disclosed ABEs comprise a bipartite NLS. The adenine base editors may contain a single adenosine deaminase domain or two adenosine deaminase domains, for instance, a wild-type adenosine deaminase and an adenosine deaminase variant. In exemplary embodiments, the disclosed ABEs contain a single adenosine deaminase domain that comprises TadA-8e.

The disclosed adenine base editors may comprise the structure: NH₂-[first nuclear localization sequence]-[adenosine deaminase domain]-[napDNAbp domain]-[second nuclear localization sequence]-COOH, wherein each instance of “]-[” indicates the presence of an optional linker sequence. This structure comprises the “ABE8e” architecture.

Non-limiting examples of evolved adenosine deaminases that accept DNA as substrates are provided below. In some embodiments, an adenosine deaminase comprises any of the following amino acid sequences, or an amino acid sequence at least 70%, at least 75%, at least 80%, at least 85%, at least 90%, at least 95%, at least 99%, or at least 99.9% identical to any of the following amino acid sequences (SEQ ID NOs: 57, 21-26, 111-118, and 122-123):

ecTadA
(SEQ ID NO: 57)
SEVEFSHEYWMRHALTLAKRAWDEREVPVGAVLVHNNRVIGEGWN
RPIGRHDPTAHAEIMALRQGGLVMQNYRLIDATLYVTLEPCVMCA
GAMIHSRIGRVVFGARDAKTGAAGSLMDVLHHPGMNHRVEITEGI
LADECAALLSDFFRMRRQEIKAQKKAQSSTD
Staphylococcus aureus TadA:
(SEQ ID NO: 21)
MGSHMTNDIYFMTLAIEEAKKAAQLGEVPIGAIITKDDEVIARAH
NLRETLQQPTAHAEHIAIERAAKVLGSWRLEGCTLYVTLEPCVMC
AGTIVMSRIPRVVYGADDPKGGCSGSLMNLLQQSNFNHRAIVDKG
VLKEACSTLLTTFFKNLRANKKSTN
Bacillus subtilis TadA:
(SEQ ID NO: 22)
MTQDELYMKEAIKEAKKAEEKGEVPIGAVLVINGEIIARAHNLRE
TEQRSIAHAEMLVIDEACKALGTWRLEGATLYVTLEPCPMCAGAV
VLSRVEKVVFGAFDPKGGCSGTLMNLLQEERFNHQAEVVSGVLEE
ECGGMLSAFFRELRKKKKAARKNLSE
Salmonella typhimurium (S. typhimurium)
TadA:
(SEQ ID NO: 23)
MPPAFITGVTSLSDVELDHEYWMRHALTLAKRAWDEREVPVGAVL
VHNHRVIGEGWNRPIGRHDPTAHAEIMALRQGGLVLQNYRLLDTT
LYVTLEPCVMCAGAMVHSRIGRVVFGARDAKTGAAGSLIDVLHHP
GMNHRVEIIEGVLRDECATLLSDFFRMRRQEIKALKKADRAEGAG
PAV
Shewanella putrefaciens (S. putrefaciens)
TadA:
(SEQ ID NO: 24)
MDEYWMQVAMQMAEKAEAAGEVPVGAVLVKDGQQIATGYNLSISQ
HDPTAHABILCLRSAGKKLENYRLLDATLYITLEPCAMCAGAMVH
SRIARVVYGARDEKTGAAGTVVNLLQHPAFNHQVEVTSGVLAEAC
SAQLSRFFKRRRDEKKALKLAQRAQQGIE
Haemophilus influenzae F3031 (H. influenzae)
TadA:
(SEQ ID NO: 25)
MDAAKVRSEFDEKMMRYALELADKAEALGEIPVGAVLVDDARNII
GEGWNLSIVQSDPTAHAEIIALRNGAKNIQNYRLLNSTLYVTLEP
CTMCAGAILHSRIKRLVFGASDYKTGAIGSRFHFFDDYKMNHTLE
ITSGVLAEECSQKLSTFFQKRREEKKIEKALLKSLSDK
Caulobacter crescentus (C. crescentus)
TadA:
(SEQ ID NO: 26)
MRTDESEDQDHRMMRLALDAARAAAEAGETPVGAVILDPSTGEVI
ATAGNGPIAAHDPTAHAEIAAMRAAAAKLGNYRLTDLTLVVTLEP
CAMCAGAISHARIGRVVFGADDPKGGAVVHGPKFFAQPTCHWRPE
VTGGVLADESADLLRGFFRARRKAKI
Geobacter sulfurreducens (G. sulfurreducens)
TadA:
(SEQ ID NO: 111)
MSSLKKTPIRDDAYWMGKAIREAAKAAARDEVPIGAVIVRDGAVI
GRGHNLREGSNDPSAHAEMIAIRQAARRSANWRLTGATLYVTLEP
CLMCMGAIILARLERVVFGCYDPKGGAAGSLYDLSADPRLNHQVR
LSPGVCQEECGTMLSDFFRDLRRRKKAKATPALFIDERKVPPEP
Streptococcus pyogenes (S. pyogenes) TadA
(SEQ ID NO: 112)
MPYSLEEQTYFMQEALKEAEKSLQKAEIPIGCVIVKDGEIIGRGH
NAREESNQAIMHAEIMAINEANAHEGNWRLLDTTLFVTIEPCVMC
SGAIGLARIPHVIYGASNQKFGGADSLYQILTDERLNHRVQVERG
LLAADCANIMQTFFRQGRERKKIAKHLIKEQSDPFD
TadA 7.10:
(SEQ ID NO: 113)
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGVRNAKTGAAGSLMDVLHYPGMNHRVEITEGI
LADECAALLCYFFRMPRQVFNAQKKAQSSTD
TadA 7.10 (V106W) (E. coli)
(SEQ ID NO: 114)
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGWRNAKTGAAGSLMDVLHYPGMNHRVEITEGI
LADECAALLCYFFRMPRQVFNAQKKAQSSTD
TadA-8e (E. coli)
(SEQ ID NO: 115)
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGVRNSKRGAAGSLMNVLNYPGMNHRVEITEGI
LADECAALLCDFYRMPRQVFNAQKKAQSSIN
TadA-8e(V106W) (E. coli)
(SEQ ID NO: 116)
SEVEFSHEYWMRHALTLAKRARDEREVPVGAVLVLNNRVIGEGWN
RAIGLHDPTAHAEIMALRQGGLVMQNYRLIDATLYVTFEPCVMCA
GAMIHSRIGRVVFGWRNSKRGAAGSLMNVLNYPGMNHRVEITEGI
LADECAALLCDFYRMPRQVENAQKKAQSSIN
Aquifex aeolicus (A. aeolicus) TadA
(SEQ ID NO: 117)
MGKEYFLKVALREAKRAFEKGEVPVGAIIVKEGEIISKAHNSVEE
LKDPTAHAEMLAIKEACRRLNTKYLEGCELYVTLEPCIMCSYALV
LSRIEKVIFSALDKKHGGVVSVENILDEPTLNHRVKWEYYPLEEA
SELLSEFFKKLRNNII
Tad1
(SEQ ID NO: 118)
SEVEFSHEYWMRHALTLAKRARDEGEVPVGAVLVLNNRVIGEGWN
RAIGLYDPTAHAEIMALRQGGLVMQNYRLIDATLYVTFEPCVMCA
GAMIHSRIGRVVFGVRNSKRGAAGSLMNVLNYPGMDHRVEITEGI
LADECAALLCDFYRMPRQVFNAQKKAQSSIN
Tad6
(SEQ ID NO: 122)
SEVEFSHEYWMRHALTLAKRARDEGEVPVGAVLVLNNRVIGEGWN
RAIGLYDPTAHAEIMALRQGGLVMQNYGLIDATLYVTFEPCVMCA
GAMIHSRIGRVVFGVRNSKRGAAGSLMNVLNYPGMDHRVEITEGI
LADECAALLCDFYRMPRQVFNAQKKAQSSIN
Tad6-SR
(SEQ ID NO: 123)
SEVEFSHEYWMRHALTLAKRARDEGEVPVGAVLVLNNRVIGEGWN
RAIGLYDPTAHAEIMALRQGGLVMQNYGLIDATLYSTFEPCVMCA
GAMIHSRIGRVVFGVRNSKRGAAGSLMNVLNYPGMDHRVEITEGI
LADECAALLCDFYRMPRRVFNAQKKAQSSIN

Aspects of the disclosure provide fusion proteins that comprise a nucleic acid programmable DNA binding protein (napDNAbp), such as any of the Nme2Cas9 variants provided herein, and one or two adenosine deaminase domains. Without wishing to be bound by any particular theory, dimerization of adenosine deaminases (e.g., in cis or in trans) may improve the ability (e.g., efficiency) of the fusion protein to modify a nucleic acid base (for example, to deaminate adenine). In some embodiments, any of the fusion proteins may comprise 2, 3, 4, or 5 adenosine deaminase domains. In some embodiments, any of the fusion proteins provided herein comprises two adenosine deaminases. In some embodiments, any of the fusion proteins provided herein contain only two adenosine deaminases. In some embodiments, the two adenosine deaminases are the same. In some embodiments, the adenosine deaminases are any of the adenosine deaminases provided herein. In some embodiments, the two adenosine deaminases are different. Other adenosine deaminase domains besides those provided herein are known in the art, and a person of ordinary skill in the art would recognize which adenosine deaminase domains could be used in the fusion proteins of the present disclosure.

The architecture of disclosed fusion proteins having a first adenosine deaminase, a second adenosine deaminase, and a napDNAbp domain (e.g., any of the Nme2Cas9 variants provided herein) may comprise any one of the following structures, where NLS is a nuclear localization sequence (e.g., any NLS provided herein), NH₂ is the N-terminus of the fusion protein, and COOH is the C-terminus of the fusion protein: NH₂-[first adenosine deaminase]-[second adenosine deaminase]-[napDNAbp]-COOH; NH2-[first adenosine deaminase]-[napDNAbp]-[second adenosine deaminase]-COOH: NH₂-[napDNAbp]-[first adenosine deaminase]-[second adenosine deaminase]-COOH: NH₂-[second adenosine deaminase]-[first adenosine deaminase]-[napDNAbp]-COOH; NH2-[second adenosine deaminase]-[napDNAbp]-[first adenosine deaminase]-COOH; NH2-[napDNAbp]-[second adenosine deaminase]-[first adenosine deaminase]-COOH.

In some embodiments, the base editor comprises the structure: NH2-[cytidine deaminase]-[Cas9 protein]-COOH; or NH2-[Cas9 protein]-[cytidine deaminase]-COOH, wherein each “]-[” in the structure indicates the presence of an optional linker sequence. In some embodiments, the base editor comprises the structure: NH₂-[first NLS]-[cytidine deaminase]-[Cas9 protein]-[second NLS]-COOH; or NH₂-first NLS]-[Cas9 protein]-[cytidine deaminase]-[second NLS]-COOH.

### Exemplary Base Editors

In some aspects, the disclosure provides adenine base editor and cytidine base editors comprising any of the disclosed Cas variants. In some embodiments, adenine base editors and cytosine base editors comprising any of the eNme2-T.1, eNme2-T.2 (SEQ ID NO: 3), eNme2-C(SEQ ID NO: 1), eNme2-C.NR (SEQ ID NO: 4), eNme2E1-2, eNme2E2-12, eNme2E3-18, and eNme2-N1-21. Cas variants pare provided. In exemplary embodiments, adenine base editors comprising eNme2-T.1, eNme2-T.2 (SEQ ID NO: 3), eNme2-C(SEQ ID NO: 1), eNme2-C.NR (SEQ ID NO: 4), and eNme2-N1-21 are provided. In particular embodiments, adenine base editors comprising eNme2-C(SEQ ID NO: 1), such as eNme2-C-ABE8e, are provided. In some embodiments, cytidine base editors comprising eNme2-T.1, eNme2-T.2 (SEQ ID NO: 3), eNme2-C(SEQ ID NO: 1), eNme2-C.NR (SEQ ID NO: 4), and eNme2-N1-21 are provided.

In exemplary embodiments, the provided base editors are any of the following ABEs: eNme2E1-2-ABE8e, eNme2E2-12-ABE8e, eNme2E3-18-ABE8e, Nme2E1-2-ABE8e, eNme2-C-ABE8e, eNme2-T.1-ABE8e, eNme2-T.2-ABE8e, eNme2-C.NR-ABE8e, and eNme2-N1-21-ABE8e. In some embodiments, the provided base editors are any of the following CBEs: eNme2-C-BE4, eNme2-T.1-BE4, eNme2-T.2-BE4, eNme2-C.NR-BE4, and eNme2N1-21-BE4.

### Exemplary Adenine Base Editor Sequences

Exemplary adenine base editors of this disclosure comprise the following base editors. For the purposes of clarity, the adenosine deaminase domain sequences are indicated in Bold, and the napDNAbp (Cas9) domain sequences are in italics and underline.

eNme2-C-ABE8e editor: NLS, linker,
TadA8e, eNme2-C
(SEQ ID NO: 312)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVENAQKKA
QSSINSGGSSGGSSGSETPGTSESATPESSGGSSGGSAAFKSNPI
NYILGLAIGIASVGWAMVEIDEEGNPIRLIDLGVRVFERAEVPKT
GDSLAMARRLARSVRRLTRRRAHRLLRARRLLKREGVLQAADFDE
NGLITSLPNTPWQLRAAALDRKLTPLEWSAVLLHLIKHRGYLSQR
KNEGETAAKELGALLKGVANNAHALQTGDFRTPAELALNKFEKES
GHIRNQRGDYSHTFSRKDLQAELILLFEKQKEFGNPHVSGGLKEG
IETLLMTQRPALSGDAVQKMLGHCTLEPTEPKAAKNTYTAERFIW
LTKLNNLRILEQGSERPLTDTERSTLMDEPYRKSKLTYAQARKLL
GLEDTAFFKGLRYGKDNAEASTLMEMKAYHAISRALEKEGLKDKK
SPLNLSSELQDEIGTAFSLFKTDEDITGRLKDRVQPEILEALLKH
ISEDKEVQISLKALRRIVPLMEQGKRYDEACAEIYGVHYGKKNTE
EKIYLPPIPADEIRNPVVLRALSQARKVINGVVRRYGSPARIHIE
TAREVGKSFKDRKEIAKRQEENRKDREKAAAKFREYFPNFVGEPK
SKDILKLRLYEQQHGKCLYSGKEINLVRLNEKGYVEIDHALPFSR
TWDDSFNNKVLVLGSENQNKGNQTPYEYENGKDNSREWQEFKARV
ETSRFPSSKKQRILLQKFDEDGFKECNLNDTRYVNRFLCQFVADH
ILLTGKGKRRVVASNGQITNLLRGFWRLRKVRAENDRHHALDAVV
VACSTVAMQQKITRFVRYKEMNAFDGKTVDKETGKYLYQKTHEPQ
PWEFFAQEVMIRVFGKPDGKPEFEEADTPEKLRTLLAEKLSSRPE
AVHEYVTPLFVSRAPNRKMSGAHKDTLRSAKRFVKHNEKISVKRV
WLTEIKLADLENMVNYKNGREIELYEALKARLEAYGGNAKQAFDP
KDNPFYKKGGQLVKAVRVEKTQKSGVLLNKKNAYTIADNGDMVRV
DVECKVDKKGKNQYFIVPIYAWQVAENILPDIDCKGYRIDDSYTF
CFSLHKYDLIAFQKDEKSKVEFAYYINCDSSSGGFYLAWHDKGSR
EQRFRISTQNLALIQKYQVNELGKEIRPCRLKKRPPVRSGGSKRT
ADGSEFEPKKKRKV
eNme2-T.1-ABB8e editor: NLS, linker,
TadA8e, eNme2-T.1
(SEQ ID NO: 313)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVENAQKKA
QSSINSGGSSGGSSGSETPGTSESATPESSGGSSGGSAAFKPNPI
NYILGLAIGIASVGWAMVEIDEEENPIRLIDLGVRVFKRAEVPKT
GDSLAMARRLARSMRRLTRRRAHRLLRARRLLKREGVLQAADFDE
NGLIKSLPNTPWQLRAAALDRKLAPLEWSAVLLHLIKHRGYLSQR
KNEGETAGKKLGALLKGVANNAHALQTGDFRTPAELALNKFEKES
GHIRNQRGDYSHTFSRKDLQAELILLFEKQKEFGNPHVSGGLKEG
IETLLMTQRPALSGDAVQKMLGHCTFEPAEPKAAKNTYTAERFIW
LTKLNNLRILEQGSERPLTDTERATLMDEPYRKSKLTYAQARKLL
GLEDTAFFKGLRYGKDNAEASTLMEMKAYHAISRALEKEGLKDKK
SPLNLSSELQDEIGTAFSLFKTDEDIAGRLKDRVQPEILEALLKN
ISEDKEVQISLKSLRRIVPLMEQGKRYDEACAEIYGDRYGKKNTE
AKTYLPPIPADEIRNPVVLRALSQTRKVINGVVRRYGSPARIHIE
TAREVGKSFKDRKEIEKRQEENRKDREKAAAKFREYFPNFVGEPK
SKDILKLRLYEQQHGKCLYSGKEINLVRLNEKGYVEIDHALPFSR
TWDDSENNKVLVLGSENQNKGNQTPYEYENGKDNPREWQEFKARV
ETSRFPRSKKQRILLQKFDEDGFKECNLNDTRYVSRFLCQFVADH
ILLTGKGKRRVFASNGQITNLLRGFWGLRKVRAENARHHALDAVV
VACSTVAMQQKITRFVRYKEMNAFDGKTIDKETGKALYQKTRFPQ
PWEFFAQEVMIRVFGKPDGKPEFEEADTPEKLRTLLAEKLSSRPE
AAHEYVTPLFVSRAPNRKMSGAHKATLRSAKRFVKHNEKVSVKRV
LLTEIKLADLENMVNYKNGREIELYEALKARLEAYGGNAKQAFDP
KDNPFYKKGGQLVKAVRVEKTQESGVLLNKKNAYTIADNGDRVRV
DVFCKVDKKGKNQYFIVPIYAWQVAENILPDIDCKGYRIDDSYTF
CESLHRYDLIAFQKDEKSKVEFAYYINCNASNGYFYLAWHDKGSK
EQQFSISTQNLVLIQKYQVSELGKEIRPCRLKKRPPVRSGGSKRT
ADGSEFEPKKKRKV
eNme2-T.2-ABESe editor: NLS, linker,
TadA8e, eNme2-T.2
(SEQ ID NO: 314)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVENAQKKA
QSSINSGGSSGGSSGSETPGTSESATPESSGGSSGGSAAFKPNPI
NYILGLAIGIASVGWAMVEIDEEENPIRLIDLGVRVFKRAEVPKT
GDSLAMARKLARSMRRLTRRRAHRLLRARRLLKREGVLQAADEDE
NGLIKSLPNTPWQLRATALDRKLAPLEWSAVLLHLIKHRGYLSQR
KNEGETANKKLGALLKGVANNAHALQTGDFRTPAELALNKFEKES
GHIRNQRGDYSHTFSRKDLQAELILLFEKQKDFGNPHVSGGLKEG
IETLLMTQRPALSGDAVQKMLGHCTFEPAEPKAAKNTYTAERFIW
LTKLNNLRILEQGSERPLTDTERATLMDEPYRKSKLTYAQARKLL
GLEDTAFFKGLRYGKDNAEASTLMEMKAYHAISRALEKEGLKDKK
SPLNLSSELQDEIGTAFSLFKTDEDIAGRLKDRVQPEILEALLKH
ISEDKEVQISLKALRRIVPLMEQGKRYDEACAEIYGDRYGKKNTE
KKIYLPPIPADEIRNPVVLRALSQARKVINGVVRRYGSPARIHIE
TAREVGKSFKDRKEIEKRQEENRKDREKAAAKFREYFPNFVGEPK
SKDILKLRLYEQQHGKCLYSGKEINLVRLNEKGYVEIDHALPFSR
TWDDSENNKYLVLGSENQNKGNQTPYEYENGKDNSREWQEFKARV
ETSRFPRSKKQRILLQKFDEDGFKECNLNDTRYVSRFLCQFVADH
ILLIGKGKRRVFASNGQITNLLRGFWGLRKVRAENARHHSLDAVV
VACSTVAMQQKITRFVRYKEMNAFDGKTIDKETGKVLHQRTHFPQ
PWEFFAQEVMIRVFGKPDGKPEFEEADTPEKLRTLLAEKLSIRPE
AVHEYVTPLFVSRAPNRKMSGAHKATLRSAKRFVKHNEKISVKRV
WLTEIKLADLENMVNYKNGREIELYEALKARLEAYGGNAKQAFDP
KDNPFYKKGGQLVKAVRVEKTQKSGVLLNKRNAYTIADNGDRVRV
DVFCKVDKKGKNQYFIVPIYAWQVAENILPDIDCKGYRIDDSYTF
CFSLHRYDLIAFQKDEKSKVEFAYYINCNASNGNFYLAWHDKGSK
EQQFCISTQNLVLIQKYQVNELGKEIRPCRMKKRPPVRSGGSKRT
ADGSEFEPKKKRKV
eNme2-C.NR-ABE8e editor: NLS, linker,
TadA8e, eNme2-C.NR
(SEQ ID NO: 315)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVENAQKKA
QSSINSGGSSGGSSGSETPGTSESATPESSGGSSGGSAAFKPNPI
NYILGLAIGIASVGWAMVEIDEEENPIRLIDLGVRVFERAEVPKT
GDSLAMARRLARSVRRLTRRRAHRLLRARRLLKREGVLQAADFDE
NGLITSLPNTPWQLRAAALDRKLTPLEWSAVLLHLIKHRGYLSQR
KNEGETAAKELGALLKGVANNAHALQTGDFRTPAELALNKFEKES
GHIRNQRGDYSHTFSRKDLQAELILLFEKQKEFGNPHVSGGLKEG
IETLLMTQRPALSGDAVQKMLGHCTLEPTEPKAAKNTYTAERFIW
LTKLNNLRILEQGSERPLTDTERSTLMDEPYRKSKLTYAQARKLL
GLEDTAFFKGLRYGKDNAEASTLMEMKAYHAISRALEKEGLKDKK
SPLNLSSELQDEIGTAFSLEKTDEDITGRLKDRVQPEILEALLKH
ISEDKFVQISLKALRRIVPLMEQGKRYDEACAEIYGVHYGKKNTE
EKIYLPPIPADEIRNPVVLRALSQARKVINGVVRRYGSPARIHIE
TAREVGKSFKDRKEIEKRQEENRKDREKAAAKFREYFPNFVGEPK
SKDILKLRLYEQQHGKCLYSGKEINLVRLNEKGYVEIDHALPFSR
TWDDSFNNKVLVLGSENQNKGNQTPYEYENGKDNSREWQEFKARV
ETSRFPRSKKQRILLQKFDEDGFKECNLNDTRYVNRFLCQFVADH
ILLIGKGKRRVFASNGQITNLLRGFWGLRKVRAENDRHHALDAVV
VACSTVAMQQKITREVRYKEMNAFDGKTIDKETGKVLHQKTHFPQ
PWEFFAQEVMIRVFGKPDGKPEFEEADTPEKLRTLLAEKLSSRPE
AVHEYVTPLFVSRAPNRKMSGAHKDTLRSAKRFVKHNEKISVKRV
WLTEIKLADLENMVNYKNGREIELYEALKARLEAYGGNAKQAFDP
KDNPFYKKGGQLVKAVRVEKTQKSGVLLNKKNAYTIADNGDMVRV
DVFCKVDKKGKNQYFIVPIYAWQVAENILPDIDCKGYRIDDSYTF
CESLHKYDLIAFQKDEKSKVEFAYYINCDSSSGGFYLAWHDKGSR
EQRFRISTQNLALIQKYQVNELGKEIRPCRLKKRPPVRSGGSKRT
ADGSEFEPKKKRKV
eNme2-N1-21-ABE8e editor: NLS, linker,
TadA8e, eNme2-N1-21
(SEQ ID NO: 316)
MKRTADGSEFESPKKKRKVSEVEFSHEYWMRHALTLAKRARDERE
VPVGAVLVLNNRVIGEGWNRAIGLHDPTAHAEIMALRQGGLVMQN
YRLIDATLYVTFEPCVMCAGAMIHSRIGRVVFGVRNSKRGAAGSL
MNVLNYPGMNHRVEITEGILADECAALLCDFYRMPRQVENAQKKA
QSSINSGGSSGGSSGSETPGTSESATPESSGGSSGGSAAFKPNPI
NYILGLDIGIASVGWAMVEIDEEENPIRLIDLGVRVFQRAEVPKT
GDSLAMARRLARSVRRLTRRRAHRLLRARRLLKREGVLQAADFDE
NGLIKSLPHTPWQLRTAALDRKLTPLEWSAVLLHLIKHRGYLSQR
KNEGETADKKLGALLKGVANNAHALQTGDFRTPAELALNKFEKES
GHIRNQRGDYSHTFSRKNLQSGLILLFEKQKEFGNPHVSGGLKEG
IETLLMTQRPALSGDAVQKMLGHCTFESAEPKAAKNTYTAERFIW
LTKLNNLRILEQGSERPLTDTERATLMDEPYRKSKLTYAQARKLL
GLEDTAFFKGLRYVKDNAEVSTLMEMKAYHAISRALEKEGLKDKK
SPLNLSSELQDEIGTAFSLEKTDEDITGRLKDRVQPEILEALLKH
ISEDKEVQISLKALRRIVPLMEQGKRYDEACAEIYGVRYGKKNTE
EKIYLPPIPADEIRNPVVLRALSQARKVINGVVRRYGSPARIHIE
TAREVGKSFKDRKEIEKRQEENRKDREKAAAKFREYFPNFVGEPK
SKDILKLRLYEQQHGKCLYSGKEINLVRLNEKGYVEIDHALPFSR
TWDDSFNNKVLVLGSENQNKGNQTPYEYENGKDNSREWQEFKARV
ETSRFPRSKKQRILLQKFDEGGFKECNLNDTRYVNRFLCQFVADH
ILLTGKGKRRVFASNGQITNLLRGFWGLRKVRAENDRHHALDAVV
VACSTVAMQQKITRFVRYKEMNAFDGKTIDKEAGKALYQKARFPQ
PWEFFAQEVMIRVFGKPDGKPEFEEADTPEKLRTLLAERLSSRPE
AVHEYVTPLFVSRAPNRKMSGAHKVILRSAKRFVKHNEKISVKRV
WLTEIKLADLENMVNYRNGREIELYEALKARLEAYGGNAKQAFDP
KDNPFYKKGGQLVKAVRVERTQERGVLLNKKNAYTIADNGDMVRV
DVFCKVGKKGKNQYFIVPIYAWQVAENILPDIDCKGYRIDDSYTF
CFSLHKYDLIAFQKDEKSKVEFAYYSYCHSSSGDFYLAWHDKGSK
EQQFRISTQNLVLIQKYQVNELGKEIRPCRLKKRPLVRSGGSKRT
ADGSEFEPKKKRKV

### Exemplary Cytosine Base Editor Sequences

Exemplary cytosine base editors of this disclosure comprise the following base editors. For the purposes of clarity, the cytidine deaminase domain sequences are indicated in Bold, and the napDNAbp (Cas9) domain sequences are in italics and underline.

eNme2-C-BE4 editor: NLS, rAPOBEC1, UGI, eNme2-C
(SEQ ID NO: 317)
MKRTADGSEFESPKKKRKVSSETGPVAVDPTLRRRIEPHEFEVFF
DPRELRKETCLLYEINWGGRHSIWRHTSQNTNKHVEVNFIEKFTT
ERYFCPNTRCSITWFLSWSPCGECSRAITEFLSRYPHVTLFIYIA
RLYHHADPRNRQGLRDLISSGVTIQIMTEQESGYCWRNFVNYSPS
NEAHWPRYPHLWVRLYVLELYCHILGLPPCLNILRRKQPQLTFFT
IALQSCHYQRLPPHILWATGLKSGGSSGGSSGSETPGTSESATPE
SSGGSSGGSAAFKSNPINYILGLAIGIASVGWAMVEIDEEGNPIR
LIDLGVRVFERAEVPKTGDSLAMARRLARSVRRLTRRRAHRLLRA
RRLLKREGVLQAADFDENGLITSLPNTPWQLRAAALDRKLTPLEW
SAVLLHLIKHRGYLSQRKNEGETAAKELGALLKGVANNAHALQTG
DFRTPAELALNKFEKESGHIRNQRGDYSHTFSRKDLQAELILLFE
KQKEFGNPHVSGGLKEGIETLLMTQRPALSGDAVQKMLGHCTLEP
TEPKAAKNTYTAERFIWLTKLNNLRILEQGSERPLTDTERSTLMD
EPYRKSKLTYAQARKLLGLEDTAFFKGLRYGKDNAEASTLMEMKA
YHAISRALEKEGLKDKKSPLNLSSELQDEIGTAFSLFKTDEDITG
RLKDRVQPEILEALLKHISFDKFVQISLKALRRIVPLMEQGKRYD
EACAEIYGVHYGKKNTEEKTYLPPIPADEIRNPVVLRALSQARKV
INGVVRRYGSPARIHIETAREVGKSFKDRKEIAKRQEENRKDREK
AAAKFREYFPNFVGEPKSKDILKLRLYEQQHGKCLYSGKEINLVR
LNEKGYVEIDHALPFSRTWDDSFNNKVLVLGSENQNKGNQTPYEY
ENGKDNSREWQEFKARVETSRFPSSKKQRILLQKEDEDGFKECNL
NDTRYVNRFLCQFVADHILLTGKGKRRVVASNGQITNLLRGFWRL
RKVRAENDRHHALDAVVVACSTVAMQQKITRFVRYKEMNAFDGKT
VDKETGKVLYQKTHEPQPWEFFAQEVMIRVFGKPDGKPEFEEADT
PEKLRTLLAEKLSSRPEAVHEYVTPLFVSRAPNRKMSGAHKDTLR
SAKRFVKHNEKISVKRVWLTEIKLADLENMVNYKNGREIELYEAL
KARLEAYGGNAKQAFDPKDNPFYKKGGQLVKAVRVEKTQKSGVLL
NKKNAYTIADNGDMVRVDVFCKVDKKGKNQYFIVPIYAWQVAENI
LPDIDCKGYRIDDSYTFCFSLHKYDLIAFQKDEKSKVEFAYYINC
DSSSGGFYLAWHDKGSREQRFRISTQNLALIQKYQVNELGKEIRP
CRLKKRPPVRSGGSGGSGGSTNLSDIIEKETGKQLVIQESILMLP
EEVEEVIGNKPESDILVHTAYDESTDENVMLLTSDAPEYKPWALV
IQDSNGENKIKMLSGGSGGSGGSTNLSDIIEKETGKQLVIQESIL
MLPEEVEEVIGNKPESDILVHTAYDESTDENVMLLTSDAPEYKPW
ALVIQDSNGENKIKMLSGGSKRTADGSEFEPKKKRKV
eNme2-T.1-BE4 editor; NLS, rAPOBEC1,
UGI, eNme2-T.1
(SEQ ID NO: 318)
MKRTADGSEFESPKKKRKVSSETGPVAVDPTLRRRIEPHEFEVFF
DPRELRKETCLLYEINWGGRHSIWRHTSQNTNKHVEVNFIEKFTT
ERYFCPNTRCSITWFLSWSPCGECSRAITEFLSRYPHVTLFIVIA
RLYHHADPRNRQGLRDLISSGVTIQIMTEQESGYCWRNFVNYSPS
NEAHWPRYPHLWVRLYVLELYCIILGLPPCLNILRRKQPQLTFFT
IALQSCHYQRLPPHILWATGLKSGGSSGGSSGSETPGTSESATPE
SSGGSSGGSAAFKPNPINYILGLAIGIASVGWAMVEIDEEENPIR
LIDLGVRVFKRAEVPKTGDSLAMARRLARSMRRLTRRRAHRLLRA
RRLLKREGVLQAADFDENGLIKSLPNTPWQLRAAALDRKLAPLEW
SAVLLHLIKHRGYLSQRKNEGETAGKKLGALLKGVANNAHALQTG
DFRTPAELALNKFEKESGHIRNQRGDYSHTFSRKDLQAELILLFE
KQKEFGNPHVSGGLKEGIETLLMTQRPALSGDAVQKMLGHCTFEP
AEPKAAKNTYTAERFIWLTKLNNLRILEQGSERPLTDTERATLMD
EPYRKSKLTYAQARKLLGLEDTAFFKGLRYGKDNAEASTLMEMKA
YHAISRALEKEGLKDKKSPLNLSSELQDEIGTAFSLFKTDEDIAG
RLKDRVQPEILEALLKNISFDKFVQISLKSLRRIVPLMEQGKRYD
EACAEIYGDRYGKKNTEAKIYLPPIPADEIRNPVVLRALSQTRKV
INGVVRRYGSPARIHIETAREVGKSFKDRKEIEKRQEENRKDREK
AAAKFREYFPNFVGEPKSKDILKLRLYEQQHGKCLYSGKEINLVR
LNEKGYVEIDHALPFSRTWDDSFNNKVLVLGSENQNKGNQTPYEY
FNGKDNPREWQEFKARVETSRFPRSKKQRILLQKFDEDGFKECNL
NDTRYVSRFLCQFVADHILLTGKGKRRVFASNGQITNLLRGFWGL
RKVRAENARHHALDAVVVACSTVAMQQKITRFVRYKEMNAFDGKT
IDKETGKALYQKTRFPQPWEFFAQEVMIRVFGKPDGKPEFEEADT
PEKLRTLLAEKLSSRPEAAHEYVTPLFVSRAPNRKMSGAHKATLR
SAKRFVKHNEKVSVKRVLLTEIKLADLENMVNYKNGREIELYEAL
KARLEAYGGNAKQAFDPKDNPFYKKGGQLVKAVRVEKTQESGYLL
NKKNAYTIADNGDRVRVDVFCKVDKKGKNQYFIVPIYAWQVAENI
LPDIDCKGYRIDDSYTFCFSLHRYDLIAFQKDEKSKVEFAYYINC
NASNGYFYLAWHDKGSKEQQFSISTQNLVLIQKYQVSELGKEIRP
CRLKKRPPVRSGGSGGSGGSTNLSDIIEKETGKQLVIQESILMLP
EEVEEVIGNKPESDILVHTAYDESTDENVMLLTSDAPEYKPWALV
IQDSNGENKIKMLSGGSGGSGGSTNLSDIIEKETGKQLVIQESIL
MLPEEVEEVIGNKPESDILVHTAYDESTDENVMLLTSDAPEYKPW
ALVIQDSNGENKIKMLSGGSKRTADGSEFEPKKKRKV
eNme2-T.2-BE4 editor: NLS, rAPOBEC1,
UGI, eNme2-T.2
(SEQ ID NO: 319)
MKRTADGSEFESPKKKRKVSSETGPVAVDPTLRRRIEPHEFEVFF
DPRELRKETCLLYEINWGGRHSIWRHTSQNTNKHVEVNFIEKFTT
ERYFCPNTRCSITWFLSWSPCGECSRAITEFLSRYPHVTLFIYIA
RLYHHADPRNRQGLRDLISSGVTIQIMTEQESGYCWRNFVNYSPS
NEAHWPRYPHLWVRLYVLELYCHILGLPPCLNILRRKQPQLTFFT
IALQSCHYQRLPPHILWATGLKSGGSSGGSSGSETPGTSESATPE
SSGGSSGGSAAFKPNPINYILGLAIGIASVGWAMVEIDEEENPIR
LIDLGVRVFKRAEVPKTGDSLAMARKLARSMRRLTRRRAHRLLRA
RRLLKREGVLQAADFDENGLIKSLPNTPWQLRATALDRKLAPLEW
SAVLLHLIKHRGYLSQRKNEGETANKKLGALLKGVANNAHALQTG
DFRTPAELALNKFEKESGHIRNQRGDYSHTFSRKDLQAELILLFE
KQKDFGNPHVSGGLKEGIETLLMTQRPALSGDAVQKMLGHCTFEP
AEPKAAKNTYTAERFIWLTKLNNLRILEQGSERPLTDTERATLMD
EPYRKSKLTYAQARKLLGLEDTAFFKGLRYGKDNAEASTLMEMKA
YHAISRALEKEGLKDKKSPLNLSSELQDEIGTAFSLFKTDEDIAG
RLKDRVQPEILEALLKHISFDKFVQISLKALRRIVPLMEQGKRYD
EACAEIYGDRYGKKNTEKKIYLPPIPADEIRNPVVLRALSQARKV
INGVVRRYGSPARIHIETAREVGKSFKDRKEIEKRQEENRKDREK
AAAKFREYFPNFVGEPKSKDILKLRLYEQQHGKCLYSGKEINLVR
LNEKGYVEIDHALPFSRTWDDSFNNKVLVLGSENQNKGNQTPYEY
ENGKDNSREWQEFKARVETSRFPRSKKQRILLQKFDEDGFKECNL
NDTRYVSRFLCQFVADHILLTGKGKRRVFASNGQITNLLRGFWGL
RKVRAENARHHSLDAVVVACSTVAMQQKITRFVRYKEMNAFDGKT
IDKETGKVLHQRTHFPQPWEFFAQEVMIRVFGKPDGKPEFEEADT
PEKLRTLLAEKLSIRPEAVHEYVTPLFVSRAPNRKMSGAHKATLR
SAKRFVKHNEKISVKRVWLTEIKLADLENMVNYKNGREIELYEAL
KARLEAYGGNAKQAFDPKDNPFYKKGGQLVKAVRVEKTQKSGVLL
NKRNAYTIADNGDRVRVDVFCKVDKKGKNQYFIVPIYAWQVAENI
LPDIDCKGYRIDDSYTFCFSLHRYDLIAFQKDEKSKVEFAYYINC
NASNGNFYLAWHDKGSKEQQFCISTQNLVLIQKYQVNELGKEIRP
CRMKKRPPVRSGGSGGSGGSTNLSDIIEKETGKQLVIDESILMLP
EEVEEVIGNKPESDILVHTAYDESTDENVMLLTSDAPEYKPWALV
IQDSNGENKIKMLSGGSGGSGGSTNLSDIIEKETGKQLVIQESIL
MLPEEVEEVIGNKPESDILVHTAYDESTDENVMLLISDAPEYKPW
ALVIQDSNGENKIKMLSGGSKRTADGSEFEPKKKRKV
eNme2-C.NR-BE4 editor: NLS, rAPOBEC1,
UGI, eNme2-C.NR
(SEQ ID NO: 320)
MKRTADGSEFESPKKKRKVSSETGPVAVDPTLRRRIEPHEFEVFF
DPRELRKETCLLYEINWGGRHSIWRHTSQNTNKHVEVNFIEKFTT
ERYFCPNTRCSITWFLSWSPCGECSRAITEFLSRYPHVTLFIYIA
RLYHHADPRNRQGLRDLISSGVTIQIMTEQESGYCWRNFVNYSPS
NEAHWPRYPHLWVRLYVLELYCIILGLPPCLNILRRKQPQLTFFT
IALQSCHYQRLPPHILWATGLKSGGSSGGSSGSETPGTSESATPE
SSGGSSGGSAAFKPNPINYILGLAIGIASVGWAMVEIDEEENPIR
LIDLGVRVFERAEVPKTGDSLAMARRLARSVRRLTRRRAHRLLRA
RRLLKREGVLQAADFDENGLITSLPNTPWQLRAAALDRKLTPLEW
SAVLLHLIKHRGYLSQRKNEGETAAKELGALLKGVANNAHALQTG
DFRTPAELALNKFEKESGHIRNQRGDYSHTFSRKDLQAELILLFE
KQKEFGNPHVSGGLKEGIETLLMTQRPALSGDAVQKMLGHCTLEP
TEPKAAKNTYTAERFIWLTKLNNLRILEQGSERPLTDTERSTLMD
EPYRKSKLTYAQARKLLGLEDTAFFKGLRYGKDNAEASTLMEMKA
YHAISRALEKEGLKDKKSPLNLSSELQDEIGTAFSLFKTDEDITG
RLKDRVQPEILEALLKHISFDKFVQISLKALRRIVPLMEQGKRYD
EACAEIYGVHYGKKNTEEKIYLPPIPADEIRNPVVLRALSQARKV
INGVVRRYGSPARIHIETAREVGKSFKDRKEIEKRQEENRKDREK
AAAKFREYFPNFVGEPKSKDILKLRLYEQQHGKCLYSGKEINLVR
LNEKGYVEIDHALPFSRTWDDSFNNKVLVLGSENQNKGNQTPYEY
FNGKDNSREWQEFKARVETSRFPRSKKQRILLQKFDEDGFKECNL
NDTRYVNRFLCQFVADHILLTGKGKRRVFASNGQITNLLRGFWGL
RKVRAENDRHHALDAVVVACSTVAMQQKITRFVRYKEMNAFDGKT
IDKETGKVLHQKTHFPQPWEFFAQEVMIRVFGKPDGKPEFEEADT
PEKLRTLLAEKLSSRPEAVHEYVTPLFVSRAPNRKMSGAHKDTLR
SAKRFVKHNEKISVKRVWLTEIKLADLENMVNYKNGREIELYEAL
KARLEAYGGNAKQAFDPKDNPFYKKGGQLVKAVRVEKTQKSGVLL
NKKNAYTIADNGDMVRVDVFCKVDKKGKNQYFIVPIYAWQVAENI
LPDIDCKGYRIDDSYTFCESLHKYDLIAFQKDEKSKVEFAYYINC
DSSSGGFYLAWHDKGSREQRFRISTQNLALIQKYQVNELGKEIRP
CRLKKRPPVRSGGSGGSGGSTNLSDIIEKETGKQLVIQESILMLP
EEVEEVIGNKPESDILVHTAYDESTDENVMLLTSDAPEYKPWALV
IQDSNGENKIKMLSGGSGGSGGSTNLSDIIEKETGKQLVIQESIL
MLPEEVEEVIGNKPESDILVHTAYDESTDENVMLLTSDAPEYKPW
ALVIQDSNGENKIKMLSGGSKRTADGSEFEPKKKRKV

In some embodiments, the base editors comprise an amino acid sequence that is at least 80%, 85%, 90%, 95%. 98%, 99%, or 99.5% identical to the amino acid sequence of any one of SEQ ID NOs: 312-320. In particular embodiments, the adenine base editor of the disclosure comprises any one of the sequences set forth as SEQ ID NOs: 312-320.

In some embodiments, any of the base editors described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of any of SEQ ID NOs: 312-320. These differences may comprise amino acids that have been inserted, deleted, or substituted relative to the reference sequence. In some embodiments, the base editors comprise an amino acid sequence having an amino acid at position 16 (or 15) of the napDNAbp domain of the base editor, that differs from that of any of SEQ ID NOs: 312-320, such as an A16D mutation. The A16D mutation is a reversion of the nickase-conferring D16A mutation.

In some embodiments, the disclosed adenosine deaminase domains contain stretches of about 50, about 75, about 100, about 125, about 150, about 175, about 200, about 300, about 400, about 500, or more than 500 consecutive amino acids in common with either of SEQ ID NOs: 312-320.

### Nuclear Localization Sequences (NLS)

In various embodiments, the Cas proteins described herein may be fused to one or more nuclear localization sequences (NLS), which help promote translocation of a protein into the cell nucleus. In some embodiments, the fusion proteins described herein may comprise one or more NLS. Such sequences are well-known in the art and can include the following examples:

| | | SEQ |
|---|---|---|
| | | ID |
| Description | Sequence | NO: |
| NLS of SV40 | PKKKRKV | 142 |
| large T-Ag | | |
| NLS | MKRTADGSEFES | 143 |
| | PKKKRKV | |
| NLS | MDSLLMNRRKFL | 144 |
| | YQFKNVRWAKGR | |
| | RETYLC | |
| NLS of | AVKRPAATKKA | 145 |
| nucleoplasmin | GQAKKKKLD | |
| NLS of EGL-13 | MSRRRKANPTKLS | 146 |
| | ENAKKLAKEVEN | |
| NLS of c-MYC | PAAKRVKLD | 147 |
| NLS of TUS- | KLKIKRPVK | 148 |
| protein | | |
| NLS of polyoma | VSRKRPRP | 149 |
| large T-Ag | | |
| NLS of | EGAPPAKRAR | 150 |
| Hepatitis D | | |
| virus antigen | | |
| NLS of murine p53 | PPQPKKKPLDGE | 151 |
| NLS of PE1 | SGGSKRTADGS | 152 |
| and PE2 | EFEPKKKRKV | |
| Bipartite sv40 nls | KRTADGSEFESP | 153 |
| | KKKRKV | |

The NLS examples above are non-limiting. The fusion proteins provided herein may comprise any known NLS sequence, including any of those described in Cokol et al., “Finding nuclear localization signals,” EMBO Rep., 2000, 1(5): 411-415; and Freitas et al., “Mechanisms and Signals for the Nuclear Import of Proteins,” Current Genomics, 2009, 10(8): 550-7, each of which are incorporated herein by reference.

In various embodiments, the fusion proteins and constructs encoding the fusion proteins disclosed herein further comprise one or more, preferably at least two, nuclear localization sequences. In certain embodiments, the fusion proteins comprise at least two NLSs. In embodiments with at least two NLSs, the NLSs can be the same NLSs, or they can be different NLSs. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a fusion protein (e.g., inserted between the encoded napDNAbp component (e.g., any of the Nme2Cas9 variants disclosed herein) and a deaminase domain (e.g., an adenosine or cytidine deaminase).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 142), MDSLLMNRRKFLYQFKNVRWAKGRRETYLC (SEQ ID NO: 144), KRTADGSEFESPKKKRKV (SEQ ID NO: 153), or KRTADGSEFEPKKKRKV (SEQ ID NO: 155). In other embodiments, NLS comprises the amino acid sequences NLSKRPAAIKKAGQAKKKK (SEQ ID NO: 27), PAAKRVKLD (SEQ ID NO: 147), RQRRNELKRSF (SEQ ID NO: 29), or NQSSNFGPMKGGNFGGRSSGPYGGGGQYFAKPRNQGGY (SEQ ID NO: 206).

In one aspect of the disclosure, a fusion protein may be modified with one or more nuclear localization sequences (NLS), preferably at least two NLSs. In certain embodiments, the fusion proteins are modified with two or more NLSs. The disclosure contemplates the use of any nuclear localization sequence known in the art at the time of the disclosure, or any nuclear localization sequence that is identified or otherwise made available in the state of the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 142)); (ii) a bipartite motif consisting of two basic domains separated by a variable number of spacer amino acids and exemplified by the Xenopus nucleoplasmin NLS (KRXXXXXXXXXXKKKL (SEQ ID NO: 154)); and (iii) noncanonical sequences such as M9 of the hnRNPA1 protein, the influenza virus nucleoprotein NLS, and the yeast Ga14 protein NLS (Dingwall and Laskey 1991).

Nuclear localization sequences appear at various points in the amino acid sequences of proteins. NLS have been identified at the N-terminus, the C-terminus, and in the central region of proteins. Thus, the disclosure provides fusion proteins that may be modified with one or more NLSs at the C-terminus and/or the N-terminus, as well as at internal regions of the fusion protein. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein to include one or more NLSs. In one aspect, the fusion proteins may be engineered to express a fusion protein that is translationally fused at its N-terminus or its C-terminus (or both) to one or more NLSs, i.e., to form base editor-NLS fusion construct. In other embodiments, a fusion protein-encoding nucleotide sequence may be genetically modified to incorporate a reading frame that encodes one or more NLSs in an internal region of the encoded base editor. In addition, the NLSs may include various amino acid linkers or spacer regions encoded between the fusion protein and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a base editor and one or more NLSs, among other components.

The fusion proteins described herein may also comprise nuclear localization sequences that are linked to the fusion protein through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the fusion protein by any suitable strategy that effectuates forming a bond (e.g., covalent linkage, hydrogen bonding) between the fusion protein and the one or more NLSs.

### UGI Domains and Heterologous Protein Domains

In some aspects, the fusion proteins (e.g., base editors) described herein may comprise one or more uracil glycosylase inhibitor (UGI) domains. In some embodiments, the fusion proteins comprise two UGI domains. The UGI domain refers to a protein that is capable of inhibiting a uracil-DNA glycosylase base-excision repair enzyme.

In some embodiments, a UGI domain comprises a wild-type UGI or a UGI as set forth in SEQ ID NO: 28, or a variant thereof. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 28. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 28. In some embodiments, a UGI comprises an amino acid sequence homologous to the amino acid sequence set forth in SEQ ID NO: 28, or an amino acid sequence homologous to a fragment of the amino acid sequence set forth in SEQ ID NO: 28. In some embodiments, proteins comprising UGI, fragments of UGI, or homologs of UGI are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 28. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 28. In some embodiments, the UGI comprises the following amino acid sequence: >sp|P14739|UNGI_BPPB2 Uracil-DNA glycosylase inhibitor MTNLSDIIEKETGKQLVIQESILMLPEEVEEVIGNKPESDILVHTAYDESTDENVMLLTS DAPEYKPWALVIQDSNGENKIKML (SEQ ID NO: 28). In some embodiments, each of the UGI domains in any of the disclosed base editors (e.g., each of two UGI domains) comprises the sequence of SEQ ID NO: 28. In some embodiments, any of the UGI domains in any of the disclosed base editors (e.g., each of two UGI domains) comprise an amino acid sequence that differs by 1, 2, 3, 4, or 5 amino acids relative to SEQ ID NO: 28.

The fusion proteins (base editors) described herein also may include one or more additional elements. In certain embodiments, an additional element may comprise an effector of base repair, such as an inhibitor of base repair.

In some embodiments, the base editors described herein may comprise one or more heterologous protein domains (e.g., about, or more than about, 1, 2, 3, 4, 5, 6, 7, 8, 9, 10, or more domains in addition to the base editor components). A base editor may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags.

Examples of protein domains that may be fused to a base editor or component thereof (e.g., the napDNAbp domain, the cytidine deaminase domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP). HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in U.S. Patent Publication No. 2011/0059502, published Mar. 10, 2011, and incorporated herein by reference in its entirety.

The reporter gene sequences that may be used with the base editors, methods and systems disclosed herein include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), HSV thymidine kinase, rpoB, may be introduced into a cell to encode a gene into which a mutation may be introduced that will confer resistance to a particular medium in a growth selection assay for the described system.

Other exemplary features that may be present are tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags. Softags (e.g., Softag 1, Softag 3), strep-tags, biotin ligase tags, F1AsH tags, V5 tags, and SBP-tags. Additional suitable sequences will be apparent to those of skill in the art. In some embodiments, the fusion protein may comprise one or more His tags.

### Linkers

The fusion proteins described herein may include one or more linkers. As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a deaminase (e.g., a cytidine deaminase or an adenosine deaminase). In some embodiments, a linker joins a Nme2Cas9 variant provided herein and a deaminase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide, or amino acid-based. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid. 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker comprises the amino acid sequence (GGGGS)ₙ (SEQ ID NO: 156), (G)ₙ (SEQ ID NO: 157), (EAAAK)ₙ (SEQ ID NO: 158), (GGS)ₙ (SEQ ID NO: 159), (SGGS)ₙ (SEQ ID NO: 160), (XP)ₙ (SEQ ID NO: 161), or any combination thereof, wherein n is independently an integer between 1 and 30, and wherein X is any amino acid. In some embodiments, the linker comprises the amino acid sequence (GGS)ₙ, wherein n is 1, 3 (SEQ ID NO: 169), or 7 (SEQ ID NO: 17). In some embodiments, the linker comprises the amino acid sequence SGSETPGTSESATPES (SEQ ID NO: 162). In some embodiments, the linker comprises the amino acid sequence SGSETPGTSESA (SEQ ID NO: 163). In some embodiments, the linker comprises the amino acid sequence SGSETPGTSESATPEGGSGGS (SEQ ID NO: 164). In some embodiments, the linker comprises the amino acid sequence SGGSSGGSSGSETPGTSESATPESSGGSSGGS (SEQ ID NO: 165). In some embodiments, the linker comprises the amino acid sequence SGGSGGSGGS (SEQ ID NO: 166). In some embodiments, the linker comprises the amino acid sequence SGGS (SEQ ID NO: 413). In other embodiments, the linker comprises the amino acid sequence SGGSSGGSSGSETPGTSESATPESAGSYPYDVPDYAGSAAPAAKKKKLDGSGSGGSSG GS (SEQ ID NO: 167). In some embodiments, the linker comprises the amino acid sequence

GGS,
(SEQ ID NO: 168)
GGSGGS,
(SEQ ID NO: 169)
GGSGGSGGS,
(SEQ ID NO: 170)
SGGSSGGSSGSETPGTSESATPESSGGSSGGSS,
(SEQ ID NO: 162)
SGSETPGTSESATPES,
or
(SEQ ID NO: 171)
SGGSSGGSSGSETPGTSESATPESAGSYPY
DVPDYAGSAAPAAKKKKLDGSGSGGSSGG
S

In certain embodiments, linkers may be used to link any of the peptides or peptide domains or moieties of the invention (e.g., a napDNAbp linked or fused to a deaminase domain). Any of the domains of the fusion proteins described herein may also be connected to one another through any of the presently described linkers.

### Guide Sequences (e.g., Guide RNAs)

The present disclosure further provides guide RNAs (gRNA) for use in accordance with the disclosed methods of editing. The disclosure provides guide RNAs that are designed to recognize target sequences. Such gRNAs may be designed to have guide sequences (or “spacers”) having complementarity to a protospacer within the target sequence.

Guide RNAs are also provided for use with one or more of the disclosed base editors, e.g., in the disclosed methods of editing a nucleic acid molecule. Such gRNAs may be designed to have guide sequences having complementarity to a protospacer within a target sequence to be edited, and to have backbone sequences that interact specifically with the napDNAbp domains of any of the disclosed base editors, such as eNme2Cas9 variant and eNme2Cas9 nickase domains of the disclosed base editors.

In various embodiments, the base editors may be complexed, bound, or otherwise associated with (e.g., via any type of covalent or non-covalent bond) one or more guide sequences. The guide sequence becomes associated or bound to the base editor and directs its localization to a specific target sequence having complementarity to the guide sequence or a portion thereof. The particular design embodiments of a guide sequence will depend upon the nucleotide sequence of a genomic target sequence (i.e., the desired site to be edited) and the type of napDNAbp (e.g., type of Cas9 protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of the napDNAbp (e.g., a Cas9 or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a gRNA comprises, a nucleic acid sequence comprising a spacer sequence and a scaffold sequence, wherein the spacer sequence comprises a nucleic acid sequence that is at least 80%, at least 85%, at least 90%, at least 95%, at least 98% or at least 99% identical to the nucleic acid sequence of any one of the spacers in Table 2. In some embodiments, the spacer sequence comprises a nucleic acid sequence that differs by about 1-10 (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-8, 3-6, 3-4, 4-10, 4-9, 4-6, 5-10, 5-8, 5-6, 6-10, 6-8, 7-10, 7-8, 8-10, or 9-10) nucleotides relative to of any one of the spacers in Table 2. In some embodiments, the spacer sequence comprises a nucleic acid sequence that differs by about 1, 2, 3, 4, 5, 6, 7, 8, 9, or 10 nucleotides relative to of any one of the spacers in Table 2. In some embodiments, a gRNA comprises, a nucleic acid sequence comprising a spacer sequence and a scaffold sequence, wherein the spacer sequence comprises a nucleic acid sequence of any one of the spacers in Table 2. In some embodiments, the scaffold sequence comprises the nucleic acid sequence of SEQ ID NO: 100. In some embodiments, the nucleic acid sequence comprises a spacer sequence and a scaffold sequence is at least 70%, at least 75%, at least 80%, at least 85%, at least 90%, at least 95%, at least 98% or at least 99% identical to the nucleic acid sequence of any one of SEQ ID NOs: 200-205. In some embodiments, the guide RNA comprises a nucleic acid sequence of any one of SEQ ID NOs: 200-205, or a nucleic acid sequence that is at least at least 70%, at least 75%, at least 80%, at least 85%, at least 90%, at least 95%, at least 98% or at least 99% identical to the nucleic acid sequence of any one of SEQ ID NOs:200-205. In some embodiments, the nucleic acid sequence comprises a spacer sequence and a scaffold sequence is identical to the nucleic acid sequence of any one of SEQ ID NOs: 200-205. In some embodiments, the nucleic acid sequence comprises a spacer sequence and a scaffold sequence is identical to the nucleic acid sequence of SEQ ID NO: 200. In some embodiments, the nucleic acid sequence comprises a spacer sequence and a scaffold sequence is identical to the nucleic acid sequence of SEQ ID NO: 201. In some embodiments, the nucleic acid sequence comprises a spacer sequence and a scaffold sequence is identical to the nucleic acid sequence of SEQ ID NO: 202. In some embodiments, the nucleic acid sequence comprises a spacer sequence and a scaffold sequence is identical to the nucleic acid sequence of SEQ ID NO: 203. In some embodiments, the nucleic acid sequence comprises a spacer sequence and a scaffold sequence is identical to the nucleic acid sequence of SEQ ID NO: 204. In some embodiments, the nucleic acid sequence comprises a spacer sequence and a scaffold sequence is identical to the nucleic acid sequence of SEQ ID NO: 205.

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence. Similarly, cleavage of a target polynucleotide sequence may be evaluated in situ by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and PA Carr & GM Church, 2009, Nature Biotechnology 27(12): 1151-62). Additional algorithms may be found in Chuai, G. et al., DeepCRISPR: optimized CRISPR guide RNA design by deep learning. Genome Biol. 19:80 (2018), and U.S. Application Ser. No. 61/836,080 and U.S. Pat. No. 8,871,445, issued Oct. 28, 2014, the entireties of each of which are incorporated herein by reference.

The guide sequence of the gRNA is linked to a tracr mate (also known as a “backbone”) sequence which in turn hybridizes to a tracr sequence. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides.

Non-limiting examples of single (DNA) polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1)
(SEQ ID NO: 333)
NNNNNNNNgtttttgtactctcaagatttaGAAAtaaatcttgc
agaagctacaaagataaggcttcatgccgaaatcaacaccctgtc
attttatggcagggtgttttcgttatttaaTTTTTT;
(2)
(SEQ ID NO: 334)
NNNNNNNNNNNNNNNNNNgtttttgtactctcaGAAAtgcagaag
ctacaaagataaggcttcatgccgaaatcaacaccctgtcatttt
atggcagggtgttttcgttatttaaTTTTTT;
(3)
(SEQ ID NO: 335)
NNNNNNNNNNNNNNNNNNNNgtttttgtactctcaGAAAtgcaga
agctacaaagataaggcttcatgccgaaatcaacaccctgtcatt
ttatggcagggtgtTTTTT;
(4)
(SEQ ID NO: 336)
NNNNNNNNNNNNNNNNNNNNgttttagagctaGAAAtagcaagtt
aaaataaggctagtccgttatcaacttgaaaaagtggcaccgagt
cggtgcTTTTTT;
(5)
(SEQ ID NO: 337)
NNNNNNNNNNNNNNNNNNNgttttagagctaGAAATAGcaagtta
aaataaggctagtccgttatcaacttgaaaaagtgTTTTTTT;
and
(6)
(SEQ ID NO: 338)
NNNNNNNNNNNNNNNNNNNNgttttagagctagAAATAGcaagtt
aaaataaggctagtccgttatcaTTTTTTTT.

In some embodiments, sequences (1) to (3) are used in combination with Cas9 from S. thermophiles CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, the guide RNAs for use in accordance with the disclosed methods of editing comprise synthetic single guide RNAs (sgRNAs) containing modified ribonucleotides. In some embodiments, the guide RNAs contain modifications such as 2′-O-methylated nucleotides and phosphorothioate linkages. In some embodiments, the guide RNAs contain 2′-O-methyl modifications in the first three and last three nucleotides, and phosphorothioate bonds between the first three and last three nucleotides. Exemplary modified synthetic sgRNAs are disclosed in Hendel A. et al., Nat. Biotechnol. 33, 985-989 (2015), incorporated herein by reference. Additional exemplary guide RNAs are described in Edraki et al., Molecular Cell 73, 714-726, incorporated herein by reference.

In some embodiments, the guide RNAs for use in accordance with the disclosed methods of editing comprise a backbone structure that is recognized by an N. meningitis Cas9 protein or domain, such as an Nme2Cas9 domain. The backbone structure (or scaffold) recognized by an Nme2Cas9 protein may comprise the sequence provided below: 5′-[guide sequence]-gttgtagctccctttctcatttcggaaacgaaatgagaaccgttgctacaataaggccgtctgaaaagatgtgccgcaacgctctgcccc ttaaagcttctgctttaaggggcatcgttta-3′ (SEQ ID NO: 100). This scaffold sequence is recognized by the NmeCas9. Nme1Cas9, Nme2Cas9, and Nme3Cas9 proteins.

In other embodiments, the guide RNAs for use in accordance with the disclosed methods of editing comprise a backbone structure that is recognized by an S. pyogenes Cas9 protein or domain, such as an SpCas9 domain of the disclosed base editors. The backbone structure recognized by an SpCas9 protein may comprise the sequence 5′-[guide sequence]-guuuuagagcuagaaauagcaaguuaaaauaaggcuaguccguuaucaacuugaaaaaguggcaccgagucggugcuuu uu-3′ (SEQ ID NO: 339), wherein the guide sequence comprises a sequence that is complementary to the protospacer of the target sequence. See U.S. Publication No. 2015/0166981, published Jun. 18, 2015, the disclosure of which is incorporated by reference herein. The guide sequence is typically 20 nucleotides long.

In other embodiments, the guide RNAs for use in accordance with the disclosed methods of editing comprise a backbone structure that is recognized by an S. aureus Cas9 protein. The backbone structure recognized by an SaCas9 protein may comprise the sequence

(SEQ ID NO: 78)
5′-[guide sequence]-guuuuaguacucuguaaugaaaauuac
agaaucuacuaaaacaaggcaaaaugcguguuuaucucgucaacugu
ggcgagauuuuuuu-3′

The sequences of suitable guide RNAs for targeting the disclosed BEs to specific genomic target sites will be apparent to those of skill in the art based on the present disclosure. Such suitable guide RNA sequences typically comprise guide sequences that are complementary to a nucleic sequence within 50 nucleotides upstream or downstream of the target nucleobase pair to be edited. Some exemplary guide RNA sequences suitable for targeting any of the provided BEs to specific target sequences are provided herein. Additional guide sequences are well known in the art and may be used with the base editors described herein. Additional exemplary guide sequences are disclosed in, for example, Jinek M., et al., Science 337:816-821(2012); Mali P, Esvelt K M & Church GM (2013) Cas9 as a versatile tool for engineering biology. Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are incorporated herein by reference.

### Methods for Generating Cas Variants and Base Editors

The invention further relates in various aspects to methods of making the disclosed improved base editors by various modes of manipulation that include, but are not limited to, codon optimization to achieve greater expression levels in a cell, and the use of nuclear localization sequences (NLSs), preferably at least two NLSs, e.g., two bipartite NLSs, to increase the localization of the expressed base editors into a cell nucleus.

### Preparation of Base Editors for Increased Expression in Cells

The base editors contemplated herein can include modifications that result in increased expression, for example, through codon optimization.

In some embodiments, the base editors (or a component thereof) is codon optimized for expression in particular cells, such as eukaryotic cells. The eukaryotic cells may be those of or derived from a particular organism, such as a mammal, including, but not limited to, human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et aL. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen: Jacobus, Pa), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

The above description is meant to be non-limiting with regard to making base editors having increased expression, and thereby increase editing efficiencies.

### Directed Evolution Methods (e.g., PACE or PANCE)

Various embodiments of the disclosure relate to providing directed evolution methods and systems (e.g., appropriate vectors, cells, phage, flow vessels, etc.) for engineering of the base editors or base editor domains of the present disclosure. The disclosure provides vector systems for the disclosed directed evolution methods to engineer any of the disclosed base editors or base editor fomains (e.g., the adenosine deaminase domains of any of the disclosed base editors).

The directed evolution vector systems and methods provided herein allow for a gene of interest (e.g., a base editor- or adenosine deaminase-encoding gene) in a viral vector to be evolved over multiple generations of viral life cycles in a flow of host cells to acquire a desired function or activity.

Reference for disclosures of phage-assisted evolution experimental methods is made to International Publication No. WO 2018/027078: International Publication No. WO 2019/079347 published Apr. 25, 2019; International Publication No. WO 2019/226593, published Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2020/214842, published Oct. 22, 2020, and International Patent Application No. PCT/US2020/033873, filed May 20, 2020, International Publication No. WO 2020/236982, published Nov. 26, 2020, and International Publication No. WO 2021/158921, the contents of each of which are incorporated herein by reference in their entireties.

Some embodiments of this disclosure provide methods of phage-assisted continuous evolution (PACE) comprising (a) contacting a population of bacterial host cells with a population of bacteriophages that comprise a gene of interest to be evolved and that are deficient in a gene required for the generation of infectious phage, wherein (1) the phage allows for expression of the gene of interest in the host cells; (2) the host cells are suitable host cells for phage infection, replication, and packaging; and (3) the host cells comprise an expression construct encoding the gene required for the generation of infectious phage, wherein expression of the gene is dependent on a function of a gene product of the gene of interest. In some embodiments, the method further comprises (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that have not been infected by the phage. In some embodiments, the method further comprises (c) isolating a mutated phage replication product encoding an evolved protein from the population of host cells.

In PACE, the gene under selection is encoded on the M13 bacteriophage genome. Its activity is linked to M13 propagation by controlling expression of gene Ill so that only active variants produce infectious progeny phage. Phage are continuously propagated and mutagenized, but mutations accumulate only in the phage genome, not the host or its selection circuit, because fresh host cells are continually flowed into (and out of) the growth vessel, effectively resetting the selection background. While previous PACE methods utilize DNA-binding selection, the continuous evolution methods described in the present disclosure are based on a functional selection for Cas9-based genome editing agents with altered PAM compatibilities, by combining elements of a DNA-binding selection with a base editing (BE) selection, such that both novel PAM recognition and subsequent BE within the protospacer are required to pass the selection.

### Development of a PACE/PANCE Evolution Circuit

Some aspects of the present disclosure relate to using a vector system for phage-assisted continuous evolution (PACE) in directed evolution of Nme2Cas9 protein. This method is widely applicable to other Cas9 proteins. In some embodiments, the PACE method comprises (a) a vector containing a nucleic acid that encodes a fusion protein; (b) a vector containing a nucleic acid that encodes a bacteriophage (phage) gene essential for phage propagation and a nucleic acid sequence encoding an in cis split intein positioned within the coding sequence of the gene; and (c) a mutagenesis plasmid. In some embodiments, the fusion protein comprises a Cas9 protein. In further embodiments, the fusion protein comprises a Nme2Cas9 protein.

In some embodiments, vector system further comprises (d) a vector containing a nucleic acid that encodes a second bacteriophage (phage) gene that prevents phage propagation and a nucleic acid sequence encoding a second in cis split intein positioned within the coding sequence of the gene. In some embodiments, the nucleic acid sequence encoding the second in cis split intein is inserted between amino acid positions 18 and 19 of the coding sequence of the second phage gene. In some embodiments, the second bacteriophage (phage) gene that prevents phage propagation is gene III (gIII)-neg. In some embodiments the vector system is in a cell. In some embodiments, the method of continuous evolution are performed in an automated continuous culture platform. In some embodiments, the automated continuous culture platform comprises a pressure regulator.

PACE enables the rapid continuous evolution of biomolecules through many generations of mutation, selection, and replication per day (FIG. 1A). During PACE, host E. coli cells continuously dilute a population of bacteriophage (selection phage, SP) containing the gene of interest (i.e., a gene encoding a variant of Nme2Cas9 protein). The gene of interest replaces gene III on the SP, which is required for progeny phage infectivity. SP containing desired gene variants trigger host-cell gene III expression from an accessory plasmid (AP). Host-cell DNA plasmids encode a genetic circuit that links the desired activity of the protein encoded in the SP to the expression of gene III on the AP. Thus, SP variants containing desired gene variants can propagate, while phage encoding inactive variants do not generate infectious progeny and are rapidly diluted out of the culture vessel (or lagoon). An arabinose-inducible mutagenesis plasmid (MP) controls the phage mutation rate.

A key to new PACE selections is linking gene III expression to the activity of interest. A low stringency selection was designed in which base editing activates T7 RNA polymerase, which transcribes gIII. A single editing event can lead to high output amplification immediately upon transcription of the edited DNA. Reference is made to International Patent Publication WO 2019/023680, published Jan. 31, 2019: Badran, A. H. & Liu, D. R. In vivo continuous directed evolution. Curr Opin. Chem. Biol. 24, 1-10 (2015); Dickinson, B. C., Packer, M. S., Badran, A. H. & Liu, D. R. A system for the continuous directed evolution of proteases rapidly reveals drug-resistance mutations. Nat. Commun. 5, 5352 (2014); Hubbard, B. P. et al. Continuous directed evolution of DNA-binding proteins to improve TALEN specificity. Nat. Methods 12, 939-942 (2015); Wang, T., Badran, A. H., Huang, T. P. & Liu, D. R. Continuous directed evolution of proteins with improved soluble expression. Nat. Chem. Biol. 14, 972-980 (2018), and Thuronyi, B. W. et al. Continuous evolution of base editors with expanded target compatibility and improved activity. Nat. Biotechnol., 1070-1079 (2019), each of which is incorporated herein by reference.

The disclosure provides vector systems for performing directed evolution of Cas proteins. In some embodiments, the vector systems comprise an expression construct that comprises a nucleic acid encoding a portion of a split intein (e.g., the N-terminal portion or the C-terminal portion of a split intein) operably linked to a nucleic acid encoding a gene required for the production of infectious phage particles, such as gIII protein (pIII protein), or a portion (e.g., fragment) thereof. In some embodiments, the gene essential for phage propagation is gene III (gIII). In some embodiments, the gIII protein comprises an in cis split intein pair connected by a polynucleotide insert sequence, at least 1 protospacer sequence, and at least 1 PAM sequence. In some embodiments, the vector comprises 2 protospacers. In some embodiments, the 2 protospacers are each flanked by a PAM sequence and comprising alternate sequence identity at PAM nucleic acid positions 1-3 and 7.

In some embodiments, the in cis intein pair is inserted between nucleotide positions 30 and 31 (28 and 29, 29 and 30, 30 and 31, 31 and 32, 32 and 33, 33 and 34, 34 and 35) of the coding sequence of gIII protein. In some embodiments, the in cis intein pair is inserted between nucleotide positions 30 and 31 of the coding sequence of gII protein. In some embodiments, the in cis intein pair is inserted between nucleotide positions 54 and 55 (50 and 51, 51 and 52, 52 and 53, 53 and 54, 54 and 55, 55 and 56, 56 and 57) of the coding sequence of gIII protein. In some embodiments, the in cis intein pair is inserted between nucleotide positions 54 and 55 of the coding sequence of gIII protein.

In some embodiments, a split-intein comprises a Nostoc punctiforme (Npu) trans-splicing DnaE intein N-terminal portion (Int-N) or an intein C-terminal portion (Int-C). In some embodiment, the nucleic acid sequence encoding an in cis split intein comprises a nucleic acid sequence encoding an Int-N, connected by a polynucleotide insert sequence to a nucleic acid sequence encoding an Int-C. In some embodiments there is 1, 2, 3, 4, 5 in cis split intein. In some embodiments there is 1 in cis split intein. In some embodiments there is 2 in cis split intein. In some embodiments, there is a first in cis split intein. In some embodiments, there is a second in cis split intein. In some embodiments, there is a first and a second in cis split intein. In some embodiments, the polynucleotide insert sequence comprises an amino acid sequence that is between 25-150 (e.g., 25-150, 25-125, 25-121, 25-100, 20-75, 25-50, 25-32, 32-150, 32-125, 32-121, 32-100, 32-75, 32-50, 50-150, 50-125, 50-121, 50-100, 50-75, 75-150, 75-125, 75-121, 75-100, 100-150, 100-125, 100-121, 121-150, 121-125, or 125-150) amino acids in length. In some embodiments, the polynucleotide insert sequence comprises an amino acid sequence that is between 32-121 amino acids in length. In some embodiments, the polynucleotide insert sequence comprises an amino acid sequence that is about 25, 32, 50, 75, 100, 121, 125, 150 amino acids in length. In some embodiments, the polynucleotide insert sequence is about 32 amino acids in length. In some embodiments, the polynucleotide insert sequence is 32 amino acids in length. In some embodiments, the polynucleotide insert sequence is about 121 amino acids in length. In some embodiments, the polynucleotide insert sequence comprises a nucleotide sequence comprising at least 1, at least 2, at least 3 or at least 4 stop codons. In some embodiments, the polynucleotide insert sequence comprises a nucleotide sequence comprising at least 1 stop codon. In some embodiments, the polynucleotide insert sequence comprises a nucleotide sequence comprising at least 2 stop codons. In some embodiments, the polynucleotide insert sequence comprises a nucleotide sequence comprising at least 3 stop codons. In some embodiments, the polynucleotide insert sequence comprises a nucleotide sequence comprising at least 4 stop codons.

In some embodiments, the polynucleotide insert sequence comprises at least 1 protospacer and at least 1 PAM sequence. In some embodiments, the protospacer comprises a nucleotide sequence comprising at least 1 disease-relevant site. In some embodiments, the disease-relevant site is a mammalian CFTR locus.

In some embodiments, the protospacer comprises a nucleotide sequence comprising at least 1, at least 2, at least 3 or at least 4 stop codons. In some embodiments, the protospacer comprises a nucleotide sequence comprising at least 1 stop codon. In some embodiments, the protospacer comprises a nucleotide sequence comprising at least 2 stop codons. In some embodiments, the protospacer comprises a nucleotide sequence comprising at least 3 stop codons. In some embodiments, the protospacer comprises a nucleotide sequence comprising at least 4 stop codons. In some embodiments, the stop codons comprise an R1162X mutation in the mammalian CFTR locus, wherein X is any amino acid other than R.

In some embodiments, the nucleic acid sequence encoding the second in cis split intein comprises a second nucleic acid sequence encoding an intein N-terminal (Int-N), connected by a second polynucleotide insert sequence to a nucleic acid sequence encoding an intein C-terminal (Int-C). In some embodiments, the polynucleotide insert sequence comprises an amino acid sequence that is between 25-150 (e.g., 25-150, 25-125, 25-121, 25-100, 20-75, 25-50, 25-32, 32-150, 32-125, 32-121, 32-100, 32-75, 32-50, 50-150, 50-125, 50-121, 50-100, 50-75, 75-150, 75-125, 75-121, 75-100, 100-150, 100-125, 100-121, 121-150, 121-125, or 125-150) amino acids in length. In some embodiments, the polynucleotide insert sequence comprises an amino acid sequence that is between 32-121 amino acids in length. In some embodiments, the polynucleotide insert sequence comprises an amino acid sequence that is about 25, 32, 50, 75, 100, 121, 125, 150 amino acids in length. In some embodiments, the polynucleotide insert sequence is about 32 amino acids in length. In some embodiments, the polynucleotide insert sequence is about 121 amino acids in length. In some embodiments, the polynucleotide insert sequence comprises a nucleotide sequence comprising at least 1, at least 2, at least 3 or at least 4 stop codons. In some embodiments, the polynucleotide insert sequence comprises a nucleotide sequence comprising at least 1 stop codon. In some embodiments, the polynucleotide insert sequence comprises a nucleotide sequence comprising at least 2 stop codons. In some embodiments, the polynucleotide insert sequence comprises a nucleotide sequence comprising at least 3 stop codons. In some embodiments, the polynucleotide insert sequence comprises a nucleotide sequence comprising at least 4 stop codons. In some embodiments, the nucleic acid that encodes a second bacteriophage (phage) gene that prevents phage propagation and a nucleic acid sequence encoding a second in cis split intein positioned within the coding sequence of the gene comprise at least 1 protospacer and at least 1 PAM sequence.

In some embodiments, the nucleic acid sequence encoding Int-N and Int-C are from N. punctiforme (Npu). In some embodiments, a split-intein is encoded by the nucleic acid sequence set forth in the exemplary sequences of SEQ ID NO: 35 (NpuN) or SEQ ID NO: 36 (NpuC).

NpuN
(SEQ ID NO: 18)
AAACAAAGCACTATTGCACTGTGTCTCAGCTACGAAACCGAAATC
TTGACCGTCGAATATGGTCTGCTGCCAATCGGCAAGATTGTTGAA
AAACGTATTGAATGTACGGTCTACTCAGTGGATAACAACGGCAAT
ATCTACACCCAGCCGGTGGCCCAGTGGCATGACCGTGGTGAACAG
GAAGTGTTCGAATATTGTCTGGAAGACGGATCTTTAATCCGTGCC
ACAAAGGATCACAAATTTATGACTGTAGATGGTCAGATGCTCCCA
ATCGACGAAATTTTTGAACGCGAATTAGACCTGATGCGCGTGGAT
AATCTCCCGAAT
NpuC
(SEQ ID NO: 19)
ATGATCAAAATTGCCACGCGTAAATATTTAGGCAAACAGAATGTT
TATGATATCGGTGTCGAGCGCGATCATAATTTCGCGCTGAAAAAC
GGCTTTATCGCCAGCAATTGTTTTAATGCACTCTTACCGTTACTG
TTTACCCCTGTGACTAAAGCC

In some embodiments, the portion of the split intein is the C-terminal portion of a split intein (e.g., the C-terminal portion of an Npu (Nostoc punctiforme) split intein). In some embodiments, the split intein C-terminal portion is positioned upstream of (e.g., 5′ relative to) the nucleic acid encoding the gene required for the production of infectious phage particles, or portion thereof. In some embodiments, the portion of the split intein is the N-terminal portion of a split intein (e.g., the N-terminal portion of an Npu split intein). In some embodiments, the split intein N-terminal portion is positioned downstream of (e.g., 3′ relative to) the nucleic acid encoding the gene required for the production of infectious phage particles, or portion thereof. In some embodiments, the nucleic acid sequence encoding the in cis split intein is inserted between amino acid positions 10 and 11 (7 and 8, 8 and 9, 9 and 10, 10 and 11, 11 and 12, 12 and 13, 13 and 14, 14 and 15, 15 and 16, 16 and 17, 17 and 18, 18 and 19, 19 and 20) of the coding sequence of the phage gene. In some embodiments, the nucleic acid sequence encoding the in cis split intein is inserted between amino acid positions 10 and 11 of the coding sequence of the phage gene. In some embodiments, the nucleic acid sequence encoding the in cis split intein is inserted between amino acid positions 18 and 19 of the coding sequence of the phage gene. In some embodiments, any of the disclosed vector system expression constructs further comprises a sequence encoding luxAB.

In some embodiments, the accessory plasmid contains a ribosome binding site (RBS), e.g., an RBS that operably controls translation of the gIII-encoding sequence. In some embodiments, the third accessory plasmid contains an RBS. In some embodiments, the RBS is weak (e.g., sd8 or r4). In some embodiments, the RBS is strong (e.g., SD8).

The split intein may be an Npu split intein. Accordingly, in some embodiments, the N-terminal and C-terminal portions of the split intein are npuC and npuN, respectively. In some embodiments, the intein is a gp41 intein, such as a gp41-8 intein. Reference is made to Carvajal-Vallejos et al. Journal of Biological Chemistry 287(34): 28686-28696 (2012), and Pinto, Thornton & Wang, Nat. Comm. (2020) 11:1529, each of which are incorporated herein by reference.

In certain embodiments, the disclosed vector systems further comprise a plurality of accessory plasmids, each comprising a unique ribosome binding site or a unique promoter. As many as five, six, seven, eight, nine, or ten variants of the third accessory plasmid may be developed with different promoters and ribosome binding sites (RBS) to tune the negative stringency of the PACE evolution, e.g., for use in a single PACE system. In certain embodiments, the vector systems further comprise a mutagenesis plasmid (“MP”). In some embodiments, the MP comprises an arabinose-inducible promoter. Mutagenesis plasmids are described, for example by International Patent Application, PCT/US2016/027795, filed Apr. 16, 2016, published as WO2016/168631 on Oct. 20, 2016, the entire contents of which are incorporated herein by reference.

In some aspects of the method the phage gene comprises a coding sequence with altered codon usage in a N-terminal region. In some embodiments, the altered codon usage comprises the N-terminal region between amino acid positions 1-18. In some embodiments, the N-terminal region comprises a sub-region of altered nucleotide homology relative to gene IV (gVI) in the phage genome. In some embodiments the in cis split intein comprises 2 protospacers, each flanked by a PAM sequence and comprising alternate sequence identity at PAM nucleic acid positions 1-3 and 7.

In some embodiments, the selection phage comprises a fusion protein comprising a TadA8e domain and a dNme2Cas9 domain connected by a polynucleotide insert and an in trans intein. In some embodiments, the in trans intein is gp41-8.

In some embodiments, a vector system is provided as part of a kit, which is useful, in some embodiments, for performing PACE to produce adenosine deaminase protein variants. For example, in some embodiments, a kit comprises a first container housing the selection phagemid of the vector system, a second container housing the first accessory plasmid of the vector system, and a third container housing the second accessory plasmid of the vector system. In some embodiments, a kit further comprises a mutagenesis plasmid. The term “mutagenesis plasmid,” as used herein, refers to a plasmid comprising a gene encoding a gene product that acts as a mutagen. In some embodiments, the gene encodes a DNA polymerase lacking a proofreading capability. Mutagenesis plasmids for PACE are generally known in the art, and are described, for example in International PCT Application No. PCT/US2016/027795, filed Sep. 16, 2016, published as WO 2016/168631; and International Publication No. WO 2021/011579, published Jan. 21, 2021, the entire contents of which are incorporated herein by reference. In some embodiments, the kit further comprises a set of written or electronic instructions for performing PACE.

In some embodiments of the directed evolution methods and systems provided herein, the viral vector or the selection phage is a filamentous phage, for example, an M13 phage, such as an M13 selection phage as described in more detail in Publication No. WO 2016/168631. In some such embodiments, the gene required for the production of infectious viral particles is the M13 gene III (gIII).

In some embodiments, the incubating of the host cells is for a time sufficient for at least 10, at least 20, at least 30, at least 40, at least 50, at least 100, at least 200, at least 300, at least 400, at least. 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, a viral vector/host cell combination is chosen in which the life cycle of the viral vector is significantly shorter than the average time between cell divisions of the host cell. Average cell division times and viral vector life cycle times are well known in the art for many cell types and vectors, allowing those of skill in the art to ascertain such host cell/vector combinations. In certain embodiments, host cells are being removed from the population of host cells contacted with the viral vector at a rate that results in the average time of a host cell remaining in the host cell population before being removed to be shorter than the average time between cell divisions of the host cells, but to be longer than the average life cycle of the viral vector employed. The result of this is that the host cells, on average, do not have sufficient time to proliferate during their time in the host cell population while the viral vectors do have sufficient time to infect a host cell, replicate in the host cell, and generate new viral particles during the time a host cell remains in the cell population. This assures that the only replicating nucleic acid in the host cell population is the viral vector, and that the host cell genome, the accessory plasmid, or any other nucleic acid constructs cannot acquire mutations allowing for escape from the selective pressure imposed.

For example, in some embodiments, the average time a host cell remains in the host cell population is about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes.

In some embodiments, the average time a host cell remains in the host cell population depends on how fast the host cells divide and how long infection (or conjugation) requires. In general, the flow rate should be faster than the average time required for cell division, but slow enough to allow viral (or conjugative) propagation. The former will vary, for example, with the media type, and can be delayed by adding cell division inhibitor antibiotics (FtsZ inhibitors in E. coli, etc.). Since the limiting step in continuous evolution is production of the protein required for gene transfer from cell to cell, the flow rate at which the vector washes out will depend on the current activity of the gene(s) of interest. In some embodiments, titratable production of the protein required for the generation of infectious particles, as described herein, can mitigate this problem. In some embodiments, an indicator of phage infection allows computer-controlled optimization of the flow rate for the current activity level in real-time.

In some embodiments, the fresh host cells comprise the accessory plasmid required for selection of viral vectors, for example, the accessory plasmid comprising the gene required for the generation of infectious phage particles that is lacking from the phages being evolved. In some embodiments, the host cells are generated by contacting an uninfected host cell with the relevant vectors, for example, the accessory plasmid and, optionally, a mutagenesis plasmid, and growing an amount of host cells sufficient for the replenishment of the host cell population in a continuous evolution experiment. Methods for the introduction of plasmids and other gene constructs into host cells are well known to those of skill in the art and the invention is not limited in this respect. For bacterial host cells, such methods include, but are not limited to, electroporation and heat-shock of competent cells.

In some embodiments, the accessory plasmid comprises a selection marker, for example, an antibiotic resistance marker, and the fresh host cells are grown in the presence of the respective antibiotic to ensure the presence of the plasmid in the host cells. Where multiple plasmids are present, different markers are typically used. Such selection markers and their use in cell culture are known to those of skill in the art, and the invention is not limited in this respect.

In particular embodiments, a first accessory plasmid comprises gene 111, and a second accessory plasmid comprises a T7 RNAP gene deactivated by a G to T mutation, which results in an early stop codon. A third accessory plasmid may comprise a nucleotide encoding a dCas9 fused at the N terminus to the C-terminal half of a fast-splicing intein. An exemplary phage plasmid may comprise a nucleotide encoding an adenosine deaminase fused at the C terminus to the N-terminal half of the fast-splicing intein. The full-length base editor is reconstituted from the two intein components.

In some embodiments, the selection marker is a spectinomycin antibiotic resistance marker. In other embodiments, the selection marker is a chloramphenicol or carbenicillin resistance marker. Cells may be transformed with a selection plasmid containing an inactivated spectinomycin resistance gene with a mutation at an active site that requires A:T to C:G editing to correct. Cells that fail to install the correct transversion mutation in the spectinomycin resistance gene will die, while cells that make the correction will survive. E. coli cells expressing an sgRNA targeting the active site mutation in the spectinomycin resistance gene and a nucleotide modification domain-dCas9 base editor are plated onto 2xYT agar with 256 μg/mL of spectinomycin. Surviving colonies (measured through CFUs) were sequenced to find consensus mutations in the base editors expressed in the evolved survivors. A similar selection assay was used to evolve adenosine deaminase activity in DNA during adenine base editor development, as described in Gaudelli, N. M. et al., Programmable base editing of A•T to G•C in genomic DNA without DNA cleavage. Nature 551, 464-471 (2017), incorporated herein in its entirety by reference.

In some embodiments, the host cell population in a continuous evolution experiment is replenished with fresh host cells growing in a parallel, continuous culture. In some embodiments, the cell density of the host cells in the host cell population contacted with the viral vector and the density of the fresh host cell population is substantially the same.

Typically, the cells being removed from the cell population contacted with the viral vector comprise cells that are infected with the viral vector and uninfected cells. In some embodiments, cells are being removed from the cell populations continuously, for example, by effecting a continuous outflow of the cells from the population. In other embodiments, cells are removed semi-continuously or intermittently from the population. In some embodiments, the replenishment of fresh cells will match the mode of removal of cells from the cell population, for example, if cells are continuously removed, fresh cells will be continuously introduced. However, in some embodiments, the modes of replenishment and removal may be mismatched, for example, a cell population may be continuously replenished with fresh cells, and cells may be removed semi-continuously or in batches.

In some embodiments, the rate of fresh host cell replenishment and/or the rate of host cell removal is adjusted based on quantifying the host cells in the cell population. For example, in some embodiments, the turbidity of culture media comprising the host cell population is monitored and, if the turbidity falls below a threshold level, the ratio of host cell inflow to host cell outflow is adjusted to effect an increase in the number of host cells in the population, as manifested by increased cell culture turbidity. In other embodiments, if the turbidity rises above a threshold level, the ratio of host cell inflow to host cell outflow is adjusted to effect a decrease in the number of host cells in the population, as manifested by decreased cell culture turbidity. Maintaining the density of host cells in the host cell population within a specific density range ensures that enough host cells are available as hosts for the evolving viral vector population, and avoids the depletion of nutrients at the cost of viral packaging and the accumulation of cell-originated toxins from overcrowding the culture.

In some embodiments, the cell density in the host cell population and/or the fresh host cell density in the inflow is about 10² cells/ml to about 10¹² cells/ml. In some embodiments, the host cell density is about 10² cells/ml, about 10³ cells/ml, about 10⁴ cells/ml, about 10⁵ cells/ml, about 5·10W cells/ml, about 10⁶ cells/ml, about 5·10⁶ cells/ml, about 10⁷ cells/ml, about 5·10⁷ cells/ml, about 10⁸ cells/ml, about 5·10⁸ cells/ml, about 10⁹ cells/ml, about 5·10⁹ cells/ml, about 10¹⁰ cells/ml, or about 5·10¹⁰ cells/ml. In some embodiments, the host cell density is more than about 10¹⁰ cells/ml.

In some embodiments, the host cell population is contacted with a mutagen. In some embodiments, the cell population contacted with the viral vector (e.g., the phage), is continuously exposed to the mutagen at a concentration that allows for an increased mutation rate of the gene of interest, but is not significantly toxic for the host cells during their exposure to the mutagen while in the host cell population. In other embodiments, the host cell population is contacted with the mutagen intermittently, creating phases of increased mutagenesis, and accordingly, of increased viral vector diversification. For example, in some embodiments, the host cells are exposed to a concentration of mutagen sufficient to generate an increased rate of mutagenesis in the gene of interest for about 10%, about 20%, about 50%, or about 75% of the time.

In some embodiments, the host cells comprise a mutagenesis expression construct, for example, in the case of bacterial host cells, a mutagenesis plasmid. In some embodiments, the mutagenesis plasmid comprises a gene expression cassette encoding a mutagenesis-promoting gene product, for example, a proofreading-impaired DNA polymerase. In other embodiments, the mutagenesis plasmid, including a gene involved in the SOS stress response, (e.g., UmuC, UmuD′, and/or RecA). In some embodiments, the mutagenesis-promoting gene is under the control of an inducible promoter. Suitable inducible promoters are well known to those of skill in the art and include, for example, arabinose-inducible promoters, tetracycline or doxycyclin-inducible promoters, and tamoxifen-inducible promoters. In some embodiments, the host cell population is contacted with an inducer of the inducible promoter in an amount sufficient to effect an increased rate of mutagenesis. For example, in some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation.

In some embodiments, diversifying the viral vector population is achieved by providing a flow of host cells that does not select for gain-of-function mutations in the gene of interest for replication, mutagenesis, and propagation of the population of viral vectors. In some embodiments, the host cells are host cells that express all genes required for the generation of infectious viral particles, for example, bacterial cells that express a complete helper phage, and, thus, do not impose selective pressure on the gene of interest. In other embodiments, the host cells comprise an accessory plasmid comprising a conditional promoter with a baseline activity sufficient to support viral vector propagation even in the absence of significant gain-of-function mutations of the gene of interest. This can be achieved by using a “leaky” conditional promoter, by using a high-copy number accessory plasmid, thus amplifying baseline leakiness, and/or by using a conditional promoter on which the initial version of the gene of interest effects a low level of activity while a desired gain-of-function mutation effects a significantly higher activity.

Detailed methods of procedures for directing continuous evolution of base editors in a population of host cells using phage particles are disclosed in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016: International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, published as WO 2019/241649 on Dec. 19, 2019, International Patent Publication WO 2019/023680, published Jan. 31, 2019, International PCT Application, PCT/US2016/027795, filed Apr. 15, 2016, published as WO 2016/168631 on Oct. 20, 2016, International Publication No. WO 2019/040935, published on Feb. 28, 2019. International Publication No. WO 2020/041751, published on Feb. 27, 2020, and International Publication No. WO 2021/011579, published Jan. 21, 2021, each of which are incorporated herein by reference.

Methods and strategies to design conditional promoters suitable for carrying out the selection strategies described herein are well known to those of skill in the art. For an overview over exemplary suitable selection strategies and methods for designing conditional promoters driving the expression of a gene required for cell-cell gene transfer, e.g., gene III (gIII), see Vidal and Legrain, Yeast n-hybrid review, Nucleic Acid Res. 27, 919 (1999), incorporated herein in its entirety.

The disclosure provides vectors for the continuous evolution processes. In some embodiments, phage vectors for phage-assisted continuous evolution are provided. In some embodiments, a selection phage is provided that comprises a phage genome deficient in at least one gene required for the generation of infectious phage particles and a gene of interest to be evolved. Reference is made to International Patent Publication No. WO 2019/023680, published Jan. 31, 2019, and No. WO 2021/011579, published Jan. 21, 2021, each of which is incorporated herein by reference.

For example, in some embodiments, the selection phage comprises an M13 phage genome deficient in a gene required for the generation of infectious M13 phage particles, for example, a full-length gill. In some embodiments, the selection phage comprises a phage genome providing all other phage functions required for the phage life cycle except the gene required for generation of infectious phage particles. In some such embodiments, an M13 selection phage is provided that comprises gI, gII, gIV, gV, gVI, gVII, gVIII, gIX, and gX genes, but not a full-length gIII gene. In some embodiments, the selection phage comprises a 3′-fragment of gIII, but no full-length gIII. The 3′-end of gIII comprises a promoter and retaining this promoter activity is beneficial, in some embodiments, for an increased expression of gVI, which is immediately downstream of the gIII 3′-promoter, or a more balanced (wild-type phage-like) ratio of expression levels of the phage genes in the host cell, which, in turn, can lead to more efficient phage production. In some embodiments, the 3′-fragment of gill gene comprises the 3′-gIII promoter sequence. In some embodiments, the 3′-fragment of gill comprises the last 180 bp, the last 150 bp, the last 125 bp, the last 100 bp, the last 50 bp, or the last 25 bp of gIII. In some embodiments, the 3′-fragment of gIII comprises the last 180 bp of gill.

M13 selection phage is provided that comprises a gene of interest in the phage genome, for example, inserted downstream of the gVIII 3′-terminator and upstream of the gill-3′-promoter. In some embodiments, an M13 selection phage is provided that comprises a multiple cloning site for cloning a gene of interest into the phage genome, for example, a multiple cloning site (MCS) inserted downstream of the gVIII 3′-terminator and upstream of the gIII-3′-promoter.

Some embodiments of this disclosure provide a vector system for continuous evolution procedures, comprising of a viral vector, for example, a selection phage, and a matching accessory plasmid. In some embodiments, a vector system for phage-based continuous directed evolution is provided that comprises (a) a selection phage comprising a gene of interest to be evolved, wherein the phage genome is deficient in a gene required to generate infectious phage; and (b) an accessory plasmid comprising the gene required to generate infectious phage particle under the control of a conditional promoter, wherein the conditional promoter is activated by a function of a gene product encoded by the gene of interest.

In some embodiments, the selection phage is an M13 phage as described herein. For example, in some embodiments, the selection phage comprises an M13 genome including all genes required for the generation of phage particles, for example, gI, gII, gIV, gV, gVI, gVII, gVIII, gIX, and gX gene, but not a full-length gIII gene. In some embodiments, the selection phage genome comprises an F1 or an M13 origin of replication. In some embodiments, the selection phage genome comprises a 3′-fragment of gill gene. In some embodiments, the selection phage comprises a multiple cloning site upstream of the gill 3′-promoter and downstream of the gVIII 3′-terminator.

In an exemplary PACE methodology, host cells each containing a mutagenesis plasmid are diluted into 5 mL Davis Rich Medium (DRM) with appropriate antibiotics and grown to an A₆₀₀ of 0.4-0.8. Cells are then used to inoculate a chemostat (60 mL), which may be maintained under continuous dilution with fresh DRM at 1-1.5 volumes per hour to keep cell density roughly constant. Lagoons are initially filled with DRM, then continuously diluted with chemostat culture for at least 2 hours before seeding with phage. A stock solution of arabinose (1 M) may be pumped directly into lagoons (10 mM final) as previously described³⁹ for 1 hour before the addition of selection phage (SP). For the first 12 hours after phage inoculation, anhydrotetracycline is present in the stock solution (3.3 μg/mL). Lagoons may be seeded at a starting titer of ˜10⁷ pfu per mL. Dilution rate may be adjusted by modulating lagoon volume (5-20 mL) and/or culture inflow rate (10-20 mUh). Lagoons may be sampled every 24 hours by removal of culture (500 μL) by syringe. Samples are centrifuged at 13,500 g for 2 minutes and the supernatant removed and stored at 4° C. Titers are evaluated by plaquing. The presence of T7 RNAP or gene III recombinant phage is monitored by plaquing on S2060 cells containing pT7-AP and no plasmid. Phage genotypes may be assessed from single plaques by diagnostic PCR. Reference is made to Miller, S. et al. Nat. Biotechnol. (2020) and Packer, M., Rees, H. & Liu, D. Nat Commun 8, 956 (2017), each of which is incorporated by reference herein in its entirety.

Some embodiments of this disclosure provide a method of non-continuous evolution of a gene of interest. In certain embodiments, the method of non-continuous evolution is PANCE. In other embodiments, the method of non-continuous evolution is an antibiotic or plate-based selection method. PANCE uses the same genetic circuit as PACE to activate phage propagation, but instead of continuously diluting a vessel, phage are manually passaged by infecting fresh host-cell culture with an aliquot from the proceeding passage. PANCE is less stringent than PACE because there is little risk of losing a weakly active phage variant during selection, and because the effective rate of phage dilution is much lower.

In some embodiments, a method of continuous evolution comprises: (a) introducing a selection phage encoding a nucleic acid that encodes a fusion protein into a flow of a population of host cells through a lagoon, wherein the population of host cells comprise a phage gene essential for phage propagation, wherein the phage gene comprises a coding sequence comprising at least 1 stop codon and an in cis split intein, wherein the phage gene essential for phage propagation is expressed in response to contacting the population of host cells with the selection phage encoding a nucleic acid that encodes the fusion protein and the at least 1 stop codon is corrected, and wherein the flow rate of the population of host cells through the lagoon permits replication of the phage with the at least 1 stop codon corrected, but not of the host cells, in the lagoon; (b) replicating and mutating the selection phage within the flow of host cells; and (c) isolating a selection phage comprising a mutated gene to be evolved from the flow of cells. In some embodiments steps a.-c. are performed in an automated continuous culture platform.

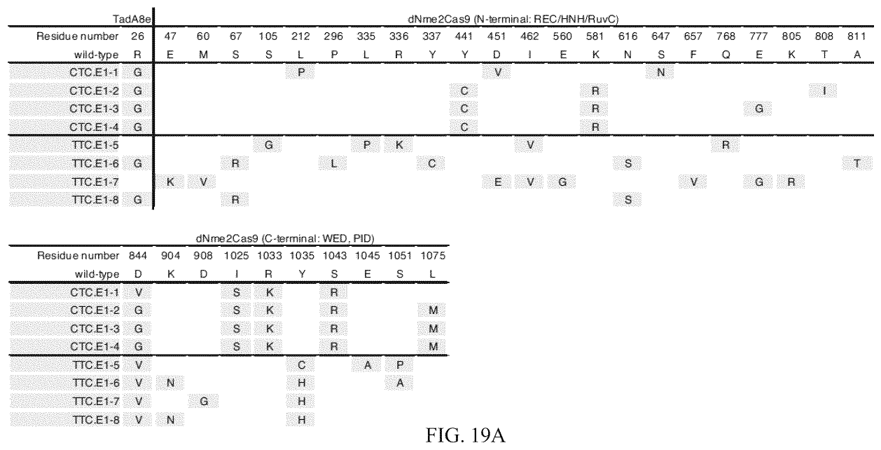

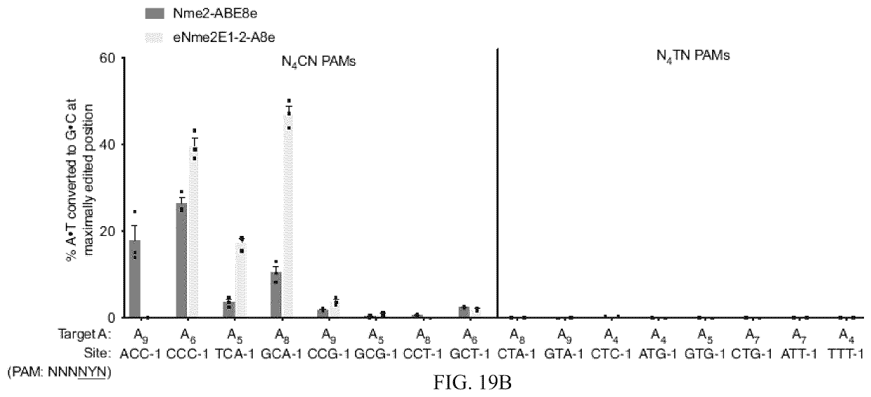

An exemplary PANCE methodology comprises first growing the host strain containing a mutagenesis plasmid of E. coli on 2xYT agar containing 0.5% glucose (w/v) along with appropriate concentrations of antibiotics until optical density reaches A₆₀₀=0.5-0.6 in a large volume. The cells are re-transformed with the mutagenesis plasmid regularly to ensure the plasmid has not been inactivated. An aliquot of a desired concentration, often 2 mL, is then transferred to a smaller flask, supplemented with 40 mM inducing agent arabinose (Ara) for the mutagenesis plasmid, and infected with the selection phage (SP). To increase the titer level, a drift plasmid may also be provided that enables phage to propagate without passing the selection. Expression is under the control of an inducible promoter and can be turned on with 0-40 ng/mL of anhydrotetracycline. Treated cultures may be split into the desired number of either 2 mL cultures in single culture tubes or 500 μL cultures in a 96-well plate and infected with selection phage (see FIG. 19). These cultures may be incubated at 37° C. for 8-12 h to facilitate phage growth, which is confirmed by determination of the phage titer, and then harvested. Following phage growth, an aliquot of infected cells is used to transfect a subsequent flask containing host E. coli. Supernatant containing evolved phage may isolated and stored at 4° C. This process may be continued until the desired phenotype is evolved for as many transfers as required, while increasing the stringency in stepwise fashion by decreasing the incubation time or titer of phage with which the bacteria is infected. In an exemplary PANCE protocol as provided herein, the process is iterated in 25 culture passages. Reference is made to Suzuki T. et al., Crystal structures reveal an elusive functional domain of pyrrolysyl-tRNA synthetase, Nat Chem Biol. 13(12): 1261-1266 (2017); and Miller, S., Wang, T. & Liu, D. Phage-assisted continuous and non-continuous evolution. Nat. Protocols 15, 4101-4127 (2020), each of which is incorporated herein in its entirety. In some embodiments, PANCE with intermittent “genetic drift”—by way of inclusion of a mutagenic genetic drift plasmid mutagenic drift plasmid—may be used. An exemplary drift plasmid may contain an anhydrotetracycline (aTc)-inducible gene.

In some embodiments, negative selection is applied during a non-continuous evolution method as described herein, by penalizing undesired activities. In some embodiments, this is achieved by causing the undesired activity to interfere with pill production. For example, expression of an antisense RNA complementary to the gill RBS and/or start codon is one way of applying negative selection, while expressing a protease (e.g., TEV) and engineering the protease recognition sites into pill is another.

Other non-continuous selection schemes for gene products having a desired activity are well known to those of skill in the art or will be apparent from the present disclosure. In certain embodiments, following the successful directed evolution of one or more components of the described base editor (e.g., a Cas9 domain or a deaminase domain), methods of making the base editors comprise recombinant protein expression methodologies known to one of ordinary skill in the art.

### Negative Selection

As described herein, aspects of the present disclosure are directed to methods and compositions concerning dual positive/negative PACE selection. As previously described, PACE is a tool useful for generating mutant Cas9 proteins with increased PAM compatibility. In further embodiments, a negative selection method may be used to increase on-target activity at desired PAM. The dual negative selection SAC-PACE circuit of the disclosure contains the vector system of SAC-PACE and an additional vector (negative accessory plasmid, “APₙ”) containing a nucleic acid that encodes a second bacteriophage gene that prevents phage propagation and a nucleic acid sequence encoding a in cis split intein positioned within the coding sequence of the gene. Previous dual selection circuits for evolving adenosine deaminase domains to be context-preferential are described in International Application No. PCT/US2022/073781, filed Jul. 15, 2022.

In some embodiments, the SAC-PACE systems of the disclosure incorporate negative selection plasmids based on sequences encoding an M13 phage gene III-negative (gIII-neg) peptide. M13 phage gene III encodes an essential coat protein that enables successful phage propagation. M13 phage gene III-negative (referred to herein as the “second bacteriophage (phage) gene”) also encodes a coat protein, but incorporation of the gene III-negative protein renders the phage incapable of infecting subsequent bacterial hosts. A negative selection plasmid can carry components that apply a negative selection pressure on editing at undesired PAMs. Undesired PAMs may include purine-rich PAMs.

In some embodiments, the nucleic acid sequence encoding a in cis split intein (referred to herein as the “second in cis split intein”) is positioned within the coding sequence of the second phage gene. In some embodiments, the nucleic acid sequence encoding the second in cis split intein contains at least 1 protospacer and at least 1 PAM sequence. In some embodiments, the nucleic acid sequence encoding the second in cis split intein is inserted between amino acid positions 18 and 19 of the coding sequence of the second phage gene. In some embodiments, the second in cis split intein contains a nucleic acid sequence encoding an intein N-terminal (Int-N), connected by a second polynucleotide insert sequence to a nucleic acid sequence encoding an intein C-terminal (Int-C). In some embodiments, the polynucleotide insert sequence comprises an amino acid sequence that is between 20-140 (e.g., 20-140, 20-120, 20-80, 20-60, 20-40, 40-140, 40-120, 40-100, 40-80, 40-60, 60-140, 60-120, 60-100, 60-80, 80-140, 80-120, 80-100, 100-140, 100-120, or 120-140) amino acids in length. In some embodiments, the polynucleotide insert sequence comprises an amino acid sequence that is between about 20, 40, 60, 80, 100, 120, or 140 amino acids in length. In some embodiments, the polynucleotide insert sequence comprises an amino acid sequence that is between about 32-121 amino acids in length. In some embodiments, the polynucleotide insert sequence contains a nucleotide sequence comprising at least 1, at least 2, at least 3 or at least 4 stop codons.

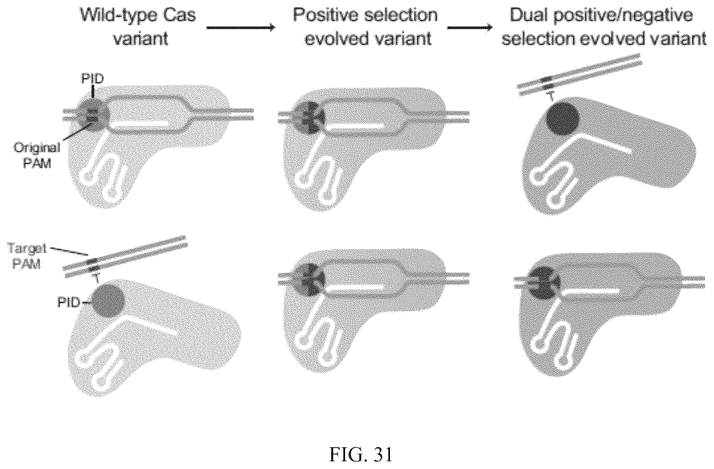

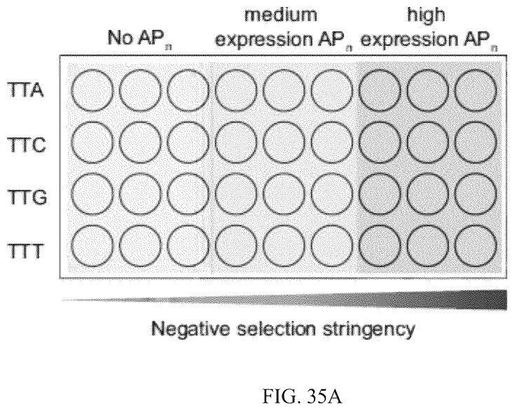

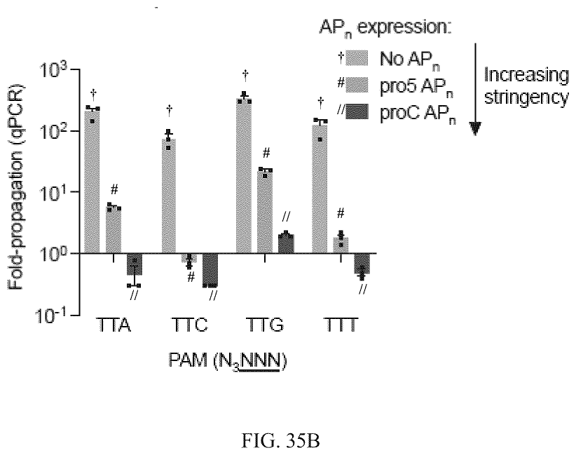

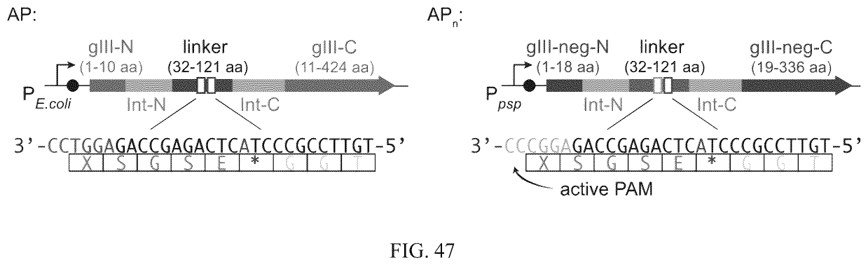

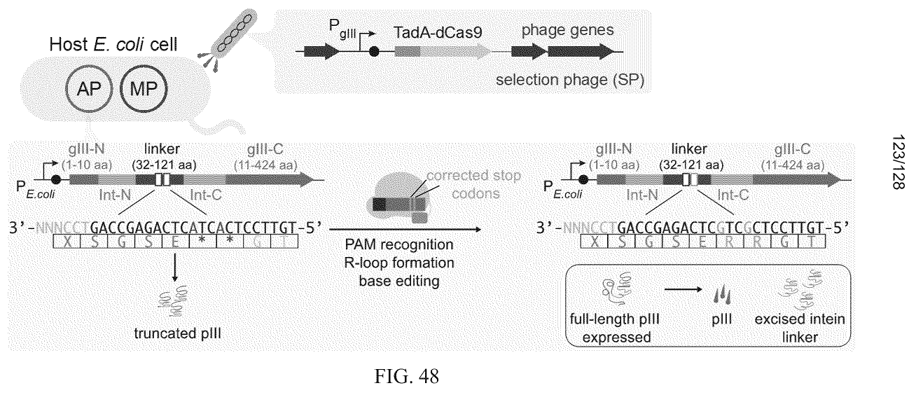

An exemplary negative selection system in SAC-PACE is shown in FIGS. 31-32, 45-50 and 52-32). Undesired PAMs readily encoded into the linker of accessory plasmid APₙ. Multiplexed negative selection is possible through multiple copies of the coding sequence. Only Cas variants capable of recognizing PAMs present on the positive accessory plasmid (AP), but not the negative accessory plasmid (APₙ) confer survival on the phages. As an example, FIG. 35A-35B shows PANCE experiments on several N₃TTN PAMs with a counterselection on N₃CCC (wild-type) PAMs.

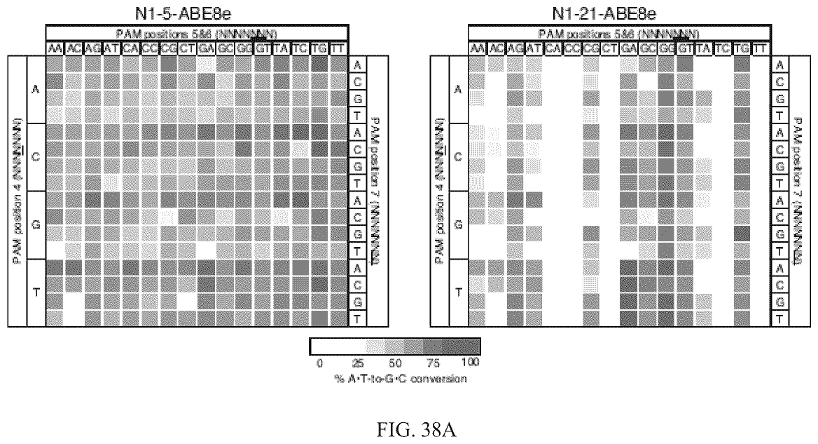

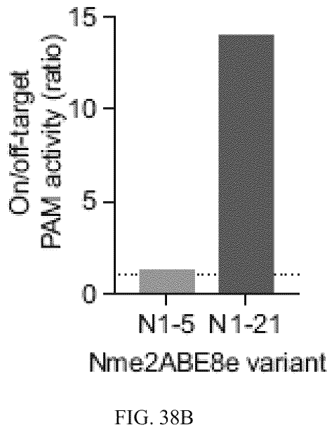

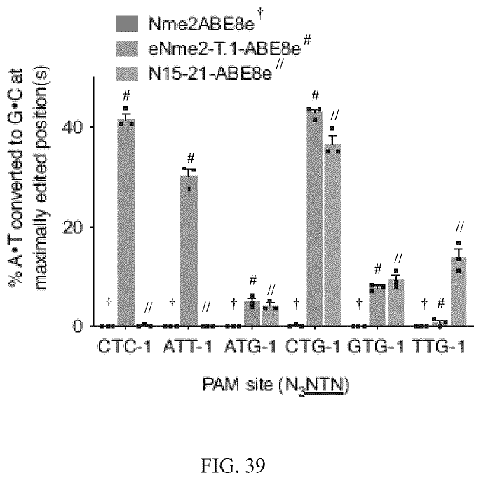

As shown in FIGS. 38A-39, the negatively selected evolved Nme2Cas9 variant, eNme2-N1-21 loses off-target activity while retaining strong target PAM activity, and in particular, 15:1 vs. 1:1 on/off-target activity.

EVOLVER and ePACE

The present disclosure provides methods, systems, and devices for high-throughput continuous directed evolution. In particular, the present disclosure provides eVOLVER-supported phage-assisted continuous evolution (ePACE) and sequence-agnostic Cas phage-assisted continuous evolution (SAC-PACE).

eVOLVER is a multi-objective, do-it-yourself platform that gives users complete freedom to define the parameters of automated culture growth experiments (e.g. temperature, culture density, media composition, etc.), and inexpensively scale them to an arbitrary size. The system is constructed using highly modular, open-source wetware, hardware, electronics and web-based software that can be rapidly reconfigured for virtually any type of automated growth experiment. eVOLVER can continuously control and monitor up to hundreds of individual cultures, collecting, assessing, and storing experimental data in real time, for experiments of arbitrary timescale. The system permits facile programming of algorithmic culture “routines”, whereby live feedbacks between the growing culture and the system couple the status of a culture (e.g. high optical density (OD)) to its automated manipulation (e.g., dilution with fresh media). By combining this programmability with arbitrary throughput scaling, the system can be used for fine resolution exploration of fitness landscapes, or determination of phenotypic distribution along multidimensional environmental selection gradients.

In some embodiments, the automated continuous culture platform comprises any of an eVOLVER unit, an Integrated Peristaltic Pump (IPP) device, media/efflux pumps, an inducer, a pressure regulator, and a solenoid bank. In further embodiments, the automated continuous culture platform comprises an eVOLVER unit, an Integrated Peristaltic Pump (IPP) device, media/efflux pumps, an inducer, a pressure regulator, and a solenoid bank.

In some embodiments, the disclosed continuous culture systems are eVOLVER systems. In some embodiments, these systems comprise programmable Smart Sleeves that house all sensors and actuators needed to control individual cultures of the parallelized culture (lagoon) system. In exemplary embodiments, the system contains sixteen (16) smart sleeves, such that selective pressure may be applied to 16 lagoons simultaneously, in a PACE experiment. In some embodiments, the disclosed systems contain an eVOLVER unit or device. In some embodiments, the disclosed systems contain a peristaltic pump array for flowing media and waste in and out of the parallelized cultures. Accordingly, in various embodiments, the disclosed continuous culture systems interface with millifluidic multiplexing devices or modules, such as high-throughput millifluidic modules that draw on principles of large-scale integration. In some embodiments, the millifluidic, peristaltic pump module of any of the disclosed systems is an Integrated Peristaltic Pumps “IPP” module or device.

In some embodiments, the disclosed continuous culture systems configuration comprises at least one stress ramp function that is overlaid on top of at least one culture fitness function, wherein the relationship between the at least one stress ramp function and the at least one fitness function responds to increased culture fitness with increased application of stress in real-time. As used herein, a “culture fitness function” refers to an output that is indicative of microbial growth or health. In some embodiments, the culture fitness function consists of one microbial fitness measurement. In other embodiments, the culture function comprises more than one fitness measurement. As used herein, the term “stress ramp function” refers to an input that applies stress on microbial growth or health. In some embodiments, the stress ramp function consists of one microbial stress. In other embodiments, the stress ramp function comprises more than one microbial stress. Examples of microbial stresses are provided below. Reference is made to US Patent Publication No. 2021/0214713; Wong et al. Nature Biotechnology, June 2018, 36(7):614-623; and Heins et al., J Vs Erp. 2019 May (147), e59652, each of which is incorporated by reference herein.

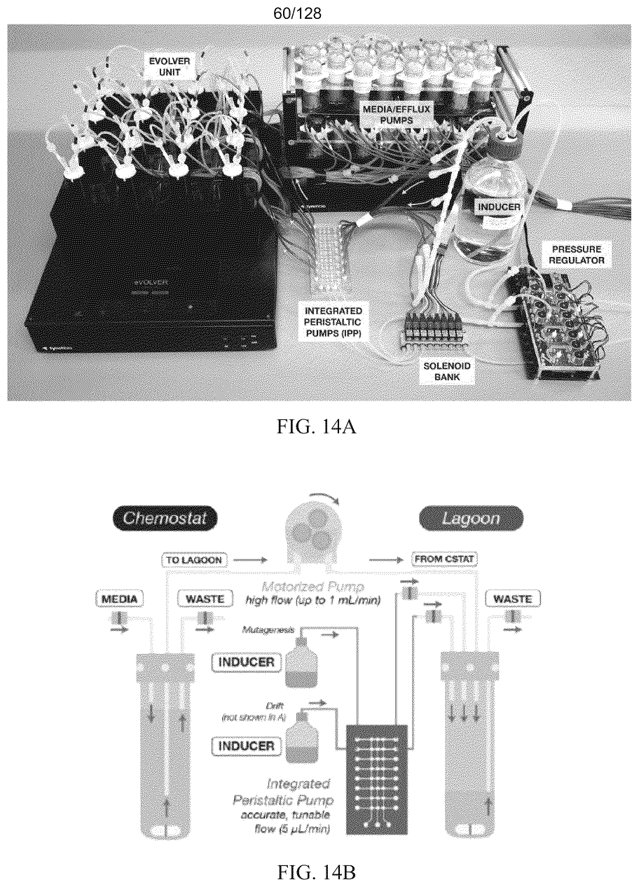

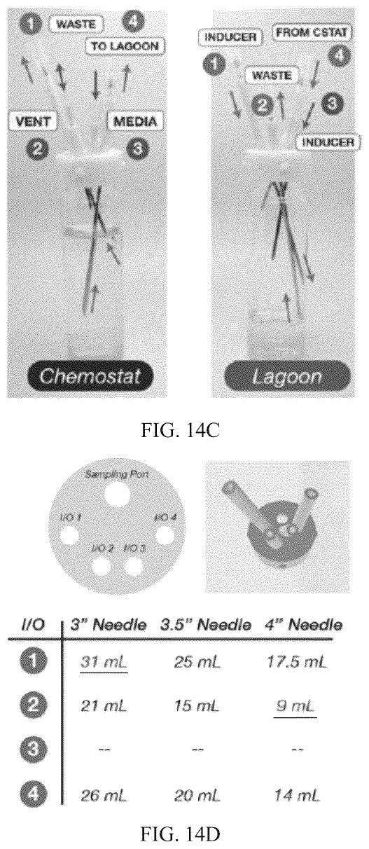

The ePACE system disclosed herein was developed based on an eVOLVER continuous culture platform, adapted to facilitate the automated operation of parallel PACE selections. As described herein, the term “ePACE” may be used to describe a system which may includes an eVOLVER continuous culture unit, IPP device, and a multi-channel pressure regulator. In some embodiments, the terms “eVOLVER unit”, “eVOLVER device” and eVOLVER continuous culture unit” may be used interchangeably. Furthermore, the do-it-yourself and open-source nature of eVOLVER allow it to be rapidly adapted and reconfigured for novel actuation elements, making it amenable to the customization necessary to run PACE (see FIGS. 14A-16D). As disclosed herein, integrating PACE and eVOLVER enabled the simultaneous execution of PACE experiments across eight different PAMs (or other selection conditions) in parallel (“parallelized PACE”). Since each evolutionary round of PACE currently requires 1-2 weeks to perform, this 8-fold increase in throughput represents a 2-4 month reduction in experimental time compared to traditional single-lagoon PACE at a 10-fold reduction in cost. A non-limiting example of an ePACE system is provided in FIG. 14A. Use of ePACE enabled large-scale parallel PACE of Nme2Cas9 towards specific PAMs, as disclosed herein. eVOLVER enabled individual programmatic control of continuous culture conditions, allowing the platform to simultaneously operate PACE chemostat cell reservoirs and lagoons on a standard lab benchtop and enabled large-scale parallelization of miniature PACE reactors.

As described herein, in an ePACE system, in some embodiments, phage continuously propagate in a fixed-volume vessel (“lagoon”) that is constantly diluted with a constant inflow of new host E. coli cells from a population maintained in a chemostat (see FIG. 14B).

A diagram of an exemplary single chemostat/lagoon pair is provided in FIG. 14B. Briefly, the fluidic movement may be as follows, fluid (e.g., media) flows into the chemostat reservoir and fluid output is either to a motorized pump or waste. In some embodiments, the motorized pump may be a high flow pump (about up to 1 mL/min). Next, the flow of fluid through the motorized pump is supplied to the lagoon reservoir. The lagoon reservoir receives additional fluid flow from the IPP device and outputs fluid to waste. In some embodiments, the IPP device may flow at a rate of 5 μl/min. At least 1 (e.g., 1 or 2) inducers may supply flow to the IPP device.

A diagram of fluidics and vials and caps for a typical ePACE setup are provided in FIG. 14C. In some embodiments, the vial caps may be provided for a fluidics unit with a set of slow (e.g., about 1 ml/m) and fast (e.g., about 1 ml/s) pump arrays for vial-to-vial/media pumping and waste pumping respectively. In some embodiments, caps may be used in combination with hypodermic needles, however other types of tubing may also be used.

A diagram of volume levels for each input/output (I/O) port on the caps with different length needles is provided in FIG. 14D. In some embodiments, an efflux needle (or pump) may be set to 5-35 (e.g., 5-35, 5-30, 5-25, 5-20, 5-15, 5-10, 10-35, 10-30, 10-25, 10-20, 10-15, 15-35, 15-30, 15-25, 15-20, 20-35, 20-30, 20-25, 25-35, 25-30, or 30-35) mL. In some embodiments the efflux needle may be set to 5, 9, 10, 14, 15, 17.5, 20, 21, 25, 26, 30, 31 or 35 mL. In some embodiments, the efflux needle for the chemostat reservoir may be set to 31 ml. In some embodiments, the efflux needle for the lagoon may be set to 9 ml.

As described herein, the term “Integrated Peristaltic Pump (IPP) device” may refer generally to a device for chemical inducer pumping that may facilitate and automate the liquid handling needs of PACE in eVOLVER. In some embodiments, the IPP device may be a millifluidic IPP device inspired by integrated microfluidics. In some embodiments, the distinction between millifluidics and microfluidics may be determined by the cross-sectional sizes of the channels, classifying them as millifluidic (larger than 1 mm), sub-millifluidic (0.5-1.0 mm), large microfluidic (100-500 μm), or microfluidic (smaller than 100 μm). See Beauchamp et al. Analytical and bioanalytical chemistry vol. 409, 18 (2017): 4311-4319, herein incorporated by reference. In some embodiments, pumping may occur at a flow rate of about 0.5 uL/s. IPPs may be inexpensively fabricated using laser cutting to achieve accurate, tunable small volume flow rates (<0.1 to 40 μL/s).

In some embodiments, the integrated peristaltic pumps (IPPs) control a flow rate. In some embodiments, the flow rate is in the range of about 0.1 to 40 (e.g., 0.1 to 40, 0.1 to 20, 0.1 to 10, 0.1 to 5, 0.1 to 1, 0.1 to 0.5, 0.5 to 40, 0.5 to 20, 0.5 to 10, 0.5 to 5, 0.5 to 1, 1 to 40, 1 to 20, 1 to 10, 1 to 5, 5 to 40, 5 to 20, 5 to 10, 10 to 20, 10 to 40, 20-40) μL/s. In some embodiments, the flow rate is in the range of less than 0.1 to 40 pus. In some embodiments, the flow rate is in the range of about 0.1 μL/s, 0.5 μL/s, 1 μL/s, 5 μL/s, 10 pus, 20 μL/s, 40 μL/s. In some embodiments, the flow rate is in the range of about 0.1 pus. In some embodiments, the flow rate is in the range of about 0.5 μL/s. In some embodiments, the flow rate is in the range of about 1 μL/s. In some embodiments, the flow rate is in the range of about 5 μL/s. In some embodiments, the flow rate is in the range of about 10 μL/s. In some embodiments, the flow rate is in the range of about 20 μL/s. In some embodiments, the flow rate is in the range of about 40 μL/s.

IPPs enable accurate and tunable metering of liquids through the sequential actuation of consecutively arranged pneumatic valves. The general architecture of IPPs may be found in US Publication No. 2010/0175767, published Jul. 15, 2010, the contents of which are incorporated by reference herein. Briefly, a main flow channel (preferably having a fluid or gas) may be crossed over by perpendicular flow channels, which are sequentially arranged and pressurized such that a membrane separating the flow channels may be depressed into the path of main flow channel, shutting off the passage of flow of fluid or gas. As such, the perpendicular “flow channel” may also be referred to as a “control line,” which actuates a single valve in the main flow channel. A plurality of such addressable valves may be joined or networked together in various arrangements to produce pumps, capable of peristaltic pumping, and other fluidic logic applications. In some embodiments, the IPP device comprises a sequential actuation of consecutively-arranged pneumatic valves. In further embodiments, the sequential actuation of consecutively-arranged pneumatic valves occurs in a “100, 010, 001” pattern, where “0” indicates “valve open,” and “1” indicates “valve closed”.

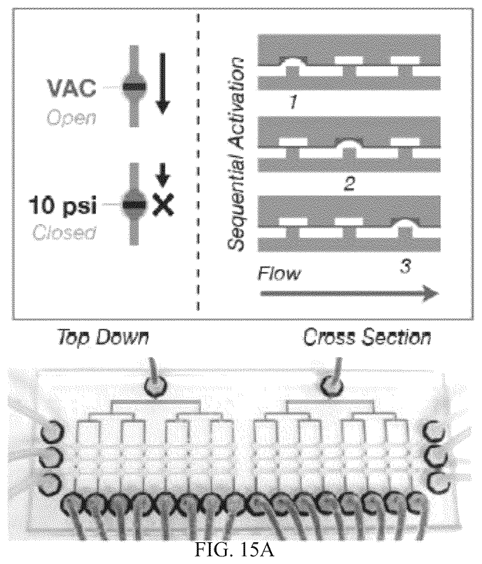

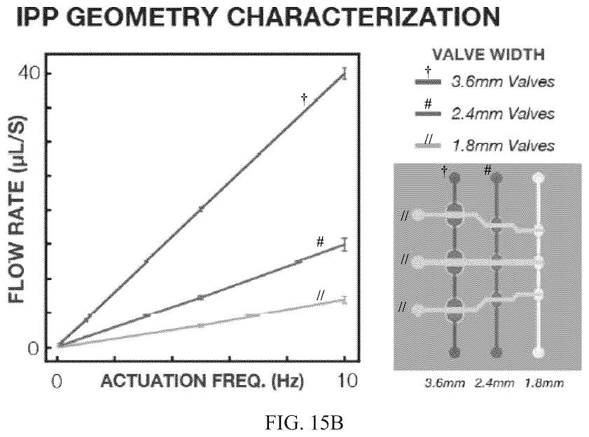

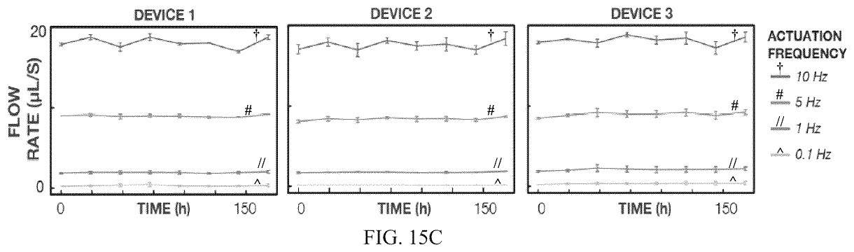

A system for peristaltic pumping is provided, as follows. A main flow channel has a plurality of generally parallel flow channels (i.e., control lines) passing thereover. By pressurizing a control line in the sequence, flow through the main flow channel is shut off under the membrane at the intersection of the control line and the main flow channel. Each of control line is separately addressable. Therefore, peristalsis may be actuated by the pattern of actuating one or more control lines in a successive pattern. For example, a successive “100, 010, 001” pattern, where “0” indicates “valve open” and “1” indicates “valve closed” (FIG. 15A). This peristaltic pattern is also known as a 120° pattern (referring to the phase angle of actuation between three valves). Other peristaltic patterns are equally possible, including 60° and 90° patterns.

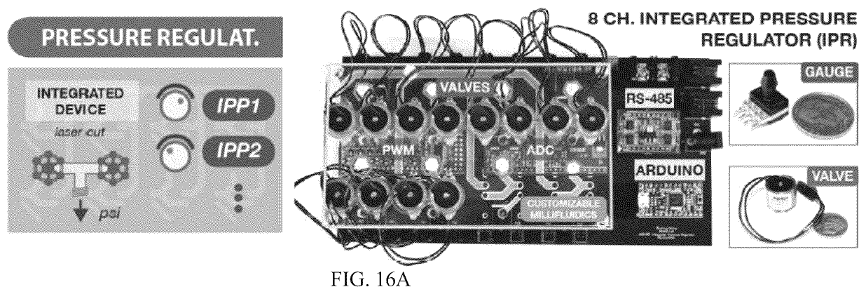

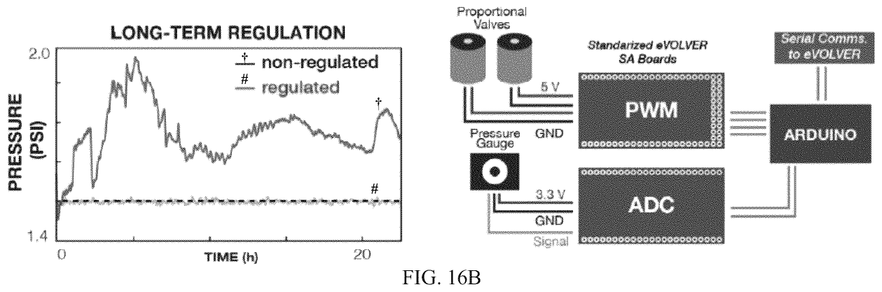

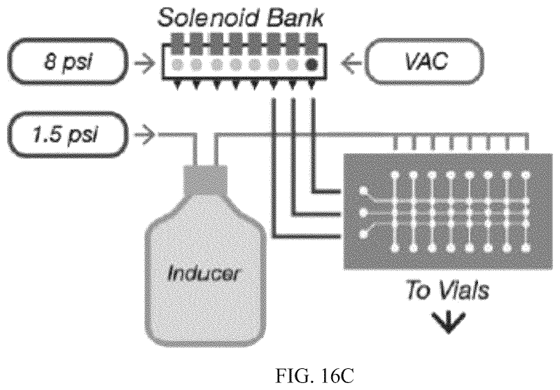

Several IPP valve sizes and cycle frequencies were characterized to generate calibration curves of achievable flow rates and verified robustness of these pumps over ˜6 million actuations over 7 days, well over the typical load necessary for PACE (see FIGS. 15A-16D). In some embodiments, IPP valve width may be 1-4 (e.g., 1-4, 1-3, 1-2, 2-4, 2-3, 3-4) mm. In some embodiments valve width may be about 1, 1.8, 2, 2.4, 3, 3.6, or 4 mm. In some embodiments valve width may be about 1.8, 2.4 or 3.6 mm.

In some embodiments, multiple IPP devices (e.g., at least one, at least two, at least three) may be used in the ePACE system. In some embodiments, IPP devices may be linked by control lines.

In some embodiments, IPP devices may be run continuously for about 100-200 (e.g., 100-200, 100-180, 100-160, 100-140, 100-120, 120-200, 120-180, 120-160, 120-140, 140-200, 140-180, 140-160, 160-200, 160-180, or 180-200) hours. In some embodiments, IPP devices may be run continuously for about 100, 120, 140, 160, 168, 180, or 200 hours. In some embodiments, IPP devices may be run continuously for about 168 hours.

In some embodiments, IPP devices may be run continuously at about 0.1-100 (e.g., 0.1-100, 0.1-50, 0.1-10, 0.1-5, 0.1-1, 1-100, 1-50, 1-10, 1-5, 5-100, 5-50, 5-10, 10-100, 10-50, or 50-100) Hz. In some embodiments, IPP devices may be run continuously at about 0.1, 1, 5, 10, 50 or 100 Hz. In some embodiments, IPP devices may be run continuously at about 10 Hz. In some embodiments, IPP devices may be run continuously for about 168 hours at about 5, 10, 15, or 20 Hz.

As described herein, the term “multi-channel pressure regulator” or “pressure regulator” may be used interchangeably to describe a device used in the present disclosure for powering IPP devices and pressurizing inducer bottles. In some embodiments, the pressure regulator comprises a modular architecture. In some embodiments, the modular architecture occurs via millifluidic interface with the valves. In some embodiments, the pressure regulator comprises multiple pressure regulators that can be chained together to regulate an arbitrary number of pressure channels. In some embodiments the pressure regulator comprises: (a) a set of two proportional valves that can limit air flow from a high-pressure source and a vent at atmospheric pressure; (b) an electronic pressure gauge on the output of the set of two proportional valves; wherein, proportional-integral-derivative (PID) control over the valves set the output pressure to any desired level between the input and atmospheric pressure. An exemplary pressure regulator is provided in FIG. 14A. In some embodiments, the pressure regulator may be an 8-channel proportional integral derivative (PID)-controlled pressure regulator (see FIG. 16A). In some embodiments, the pressure regulator has up to 16 (e.g., up to 20, up to 16, up to 14, up to 10, up to 8, up to 6, up to 4) proportional valves that can be used for pressure regulation up to 8 (up to 10, up to 8, up to 6 up to 4) channels. In some embodiments, the pressure regulator has up to 16 proportional valves that can be used for pressure regulation up to 8 channels. Compared to a manually set valve, the PID controlled pressure regulator can maintain pressure at a set value over a period of time. For example, the set value may be 1.5 psi (see FIG. 16B). An exemplary schematic of an eVOLVER pressure regulator is also shown in FIG. 16B. The eVOLVER pressure regulator may have proportional valves, each controlled via “pulse-width modulation” (PWM) using a standard eVOLVER PWM board. A single PWM board can control 16 valves simultaneously, enabling control of eight individual pressure lines. Multiple PWM board devices may be chained together to regulate arbitrary numbers of pressure channels. Electrical pressure gauge readouts are connected to a standard eVOLVER analog-to-digital (ADC) converter. Both PWM and ADC boards are connected to a SAMD21 Arduino microcontroller which controls valve open/closeness and reads data from the gauges. The microcontroller receives commands from and sends data to the eVOLVER via serial communication protocol.

As shown in FIG. 16C, a schematic of pressure regulation for ePACE, the IPP devices are powered by 8 psi provided by the pressure regulator and standard lab bench vacuum. Inducer bottles receive 1.5 psi. Pressurized media bottles can achieve higher flow rates than un-pressurized media bottles at different volumes of media (e.g., 100 mL, or 1000 mL) (See FIG. 16D).

The eVOLVER systems and methods disclosed herein are more customizable and massively parallel than existing PACE systems and methods. In exemplary ePACE methods of the disclosure, experiments may be conducted in 8 lagoons, 16 lagoons, 32 lagoons, or more than 32 lagoons simultaneously. These systems utilize customized millifluidic integrated peristaltic pumps (IPPs) that are inexpensively manufactured using laser cutting to achieve accurate, tunable small volume flow rates (<0.1 to 40 μL/s).

The fabrication method of eVOLVER utilizes laser-cut acrylic as the fluidic and control layers, with an adhesive used to bond materials. This differs from the thermal and chemical bonding methods of existing systems. Silicone is used in the elastomer membrane. This method brings both the cost of manufacturing the devices and the time it takes down considerably from microfluidics systems.

In some aspects, the disclosure relates to a continuous culture system that is configured for the testing of mutational stability of engineered circuit variants (e.g., assaying how long it takes for a circuit to inactivate or lose at least some portion of its function). A major focus of synthetic biology has been on engineering synthetic regulatory circuits to enable user-defined control of cellular function. Circuits engineered in E. coli, yeast, and other microorganisms often impose a fitness burden on their host cells and may be lost or mutated over time. Little work has gone into engineering circuits that are either robust to mutation or minimize host-cell burden. By the same token, efforts to engineer strains that can accommodate circuits without mutating them have not been undertaken.

In some aspects, the disclosure relates to a continuous culture system that is configured to assay circuit stability by growing at least one microbial cell comprising at least one circuit (or circuit library) and then assessing mutations that accrue to either the at least one circuit or the genome of the host microbial cell. In some embodiments, the at least one microbial cell comprising at least one circuit (or circuit library) is grown under stress. By selecting for circuit variants or strain backgrounds that render the circuit resistant to inactivation or loss of function, engineering rules for circuit stability can be determined.

In some embodiments, any of the disclosed continuous culture systems or platforms contain any of an eVOLVER unit, an Integrated Peristaltic Pump (IPP) device, media/efflux pumps, an inducer, a pressure regulator, and a solenoid bank (e.g., 3-way solenoid valves). In some embodiments, the disclosed continuous culture platform contains more than one, more than two, more than three, more than four, five, or six of these components. In some embodiments, the disclosed platforms contain each of an eVOLVER unit, an Integrated Peristaltic Pump (IPP) device, media/efflux pumps, an inducer, a pressure regulator, and a solenoid bank. In various embodiments, any of the disclosed platforms contain a fluidic layer and a control layer. In some embodiments, the automated continuous culture platform is comprised of a fluidic layer and a control layer. These two layers may be fabricated using a laser-cutting method or using an acrylic material. In some embodiments, the fluidic layer and the control layer are fabricated using a laser-cutting method. In some embodiments, the IPP device is manufactured using laser-cutting method. In some embodiments, the fluidic layer and the control layer are fabricated using an acrylic material. Any of the disclosed automated continuous culture platforms may be bonded using an adhesive material.

In some aspects, the disclosure relates to a method of testing the mutational stability of a microbial cell that comprises an engineered circuit. In some embodiments, the method relates to culturing at least one fluidic microbial culture in a continuous culture system and determining the time required for an engineered circuit to inactivate after subjecting a microbial cell to a dynamic environment, wherein the at least one microbial culture is exposed to a stress ramp function which is overlaid on top of a culture fitness function, and increasing the amount of stress applied to the at least one microbial culture in response to the increased fitness of the at least one microbial culture. As used herein, the term “inactivate” refers to a decrease in the output of an engineered circuit by at least about 20%, 25%, 30%, 40%, 50%, 60%, 70%, 75%, 80%, 90%, 95%. 99% or more than 99% relative to the output prior to application of the stress.

In some embodiments, microbial fitness is calculated in real-time. In some embodiments, the method evolves both the circuit and the microbial host cell. Engineered circuits, such as engineered gene circuits for expressing one or more outputs (such as proteins) in response to one or more signals, are known in the art. In some aspects, the disclosure relates to a method of testing the stability of at least one multi-species microbial community. In some embodiments, the method relates to culturing at least one fluidic multi-species microbial culture in a continuous culture system and determining the fitness of each species independently after subjecting a microbial cell to a dynamic environment, wherein the at least one microbial culture is exposed to a stress ramp function which is overlaid on top of a culture fitness function, and increasing the amount of stress applied to the at least one microbial culture in response to the increased fitness of the at least one microbial culture. In some embodiments, microbial fitness is calculated in real-time.

In some aspects, the disclosure relates to a method of constructing a multi-species community. In some embodiments, the method relates to culturing a multi-species microbial community in a continuous culture system, wherein the multi-species community comprises microbial strains that comprise engineered circuits that facilitate cell-cell interactions. A multi-species microbial community can include two or more microbial strains (of which none, some or all may include engineered circuits), such as 2, 3, 4, 5, 6, 7, 8, 9, 10, or more microbial strains. In some embodiments, the multispecies microbial community is subjected to a dynamic environment, wherein the microbial community is exposed to a stress ramp function which is overlaid on top of a culture fitness function, and increasing the amount of stress applied to the microbial community in response to outputs from the engineered circuits. In some embodiments, the output is calculated in real-time.

In some embodiments, the variants generated through an ePACE campaign, as disclosed herein, may be validated. The ability of a variant to bind a novel PAM may be validated using a BE-PPA profiling assay. BE-PPA profiling assay methods for adenine base editors and cytosine base editors are provided herein.

In some embodiments of the disclosed profiling assay methods, methods are provided that comprise transforming cells with a base editing (BE)-expressing plasmid (BP) and a library plasmid (LP), and further subjecting these cells to (a) an induction, (b) signal amplification, (c) harvesting, and (d) sequence analysis. In some embodiments, the BP comprises a guide RNA, such as a sgRNA, a promoter, and/or a base editor construct. The base editor construct may encode an adenine base editor, or it may encode a cytosine base editor. In some embodiments, the base editor construct may encode a base editor that is not an ABE or a CBE. In some embodiments, the promoter is a pBAD. The library plasmid (LP) may comprise a protospacer, a target base and/or a PAM library. In some embodiments, the sequence analysis of the disclosed methods comprises a CRISPResso2 analysis. In some embodiments, the sequence analysis step (d) of the disclosed PPA methods comprises a CRISPResso2 analysis.

### Vectors

Several aspects of the making and using the base editors of the disclosure relate to vector systems comprising one or more vectors encoding the base editors. Vectors may be designed to clone and/or express the base editors of the disclosure. Vectors may also be designed to transfect the base editors of the disclosure into one or more cells, e.g., a target diseased eukaryotic cell for treatment with the base editor systems and methods disclosed herein.

Vectors may be designed for expression of base editor transcripts (e.g. nucleic acid transcripts, proteins, or enzymes) in prokaryotic or eukaryotic cells. For example, base editor transcripts may be expressed in bacterial cells such as Escherichia coli, insect cells (using baculovirus expression vectors), yeast cells, plant cells, or mammalian cells. Suitable host cells are discussed further in Goeddel, Gene Expression Technology: Methods In Enzymology 185, Academic Press. San Diego, Calif. (1990). Alternatively, expression vectors encoding one or more base editors described herein may be transcribed and translated in vitro, for example using T7 promoter regulatory sequences and T7 polymerase. Exemplary vectors of this disclosure include the eNme2-C-ABE8e vector (Addgene No. 185667), eNme2-C-BE4 vector (Addgene No. 183679), eNme2-T.1-ABE8e vector (Addgene No. 185668), eNme2-T.2-ABE8e vector (Addgene No. 185669), and eNme2-C-NR-ABE8e vector.

Exemplary vectors used in the Examples of this disclosure are provided in Table 2, and include the pTPH418b, pTPH405, pTPH405c, and pTPH412 vectors (SEQ ID NOs: 7-10, respectively). The vectors of this disclosure may comprise a nucleic acid sequence that is at least 80%, 85%, 90%, 92.5%, 95%, 96%, 97%, 98%, or 99% identical to any of SEQ ID NOs: 7-10.

pTPH418b
(SEQ ID NO: 7)
TGAGATCCTTTTTTTCTGCGCGTAATCTGCTGCTTGCAAACAAAA
AAACCACCGCTACCAGCGGTGGTTTGTTTGCCGGATCAAGAGCTA
CCACCTCTTTTTCCGAAGGTAACTGGCTTCAGCAGAGCGCAGATA
CCAAATACTGTCCTTCTAGTGTAGCCGTAGTTAGGCCACCACTTC
AAGAACTCTGTAGCACCGCCTACATACCTCGCTCTGCTAATCCTG
TTACCAGTGGCTGCTGCCAGTGGCGATAAGTCGTGTCTTACCGGG
TTGGACTCAAGACGATAGTTACCGGATAAGGCGCAGCGGTCGGGC
TGAACGGGGGGTTCGTGCACACAGCCCAGCTTGGAGCGAACGACC
TACACCGAACTGAGATACCTACAGCGTGAGCTATGAGAAAGCGCC
ACGCTTCCCGAAGGGAGAAAGGCGGACAGGTATCCGGTAAGCGGC
AGGGTCGGAACAGGAGAGCGCACGAGGGAGCTTCCAGGGGGAAAC
GCCTGGTATCTTTATAGTCCTGTCGGGTTTCGCCACCTCTGACTT
GAGCGTCGATTTTTGTGATGCTCGTCAGGGGGGCGGAGCCTATGG
AAAAACGCCAGCAACGCGGCCTTTTTACGGTTCCTGGCCTTTTGC
TGGCCTTTTGCTCACATGTTCTTTCCTGCGTTATCCCCTGATTCT
GTGGATAACCGTATTACCGCCTTTGAGTGAGCTGATACCGCTCGC
CGCAGCCGAACGACCGAGCGCAGCGAGTCAGTGAGCGAGGAAGCG
GAAGAGCGCCTGATGCGGTATTTTCTCCTTACGCATCTGTGCGGT
ATTTCACACCGCATATGGTGCACTCTCAGTACAATCTGCTCTGAT
GCCGCATAGTTAAGCCAGTATACACTCCGCTATCGCTACGTGACT
GGGTCATGGCTGCGCCCCGACACCCGCCAACACCCGCTGACGCGC
CCTGACGGGCTTGTCTGCTCCCGGCATCCGCTTACAGACAAGCTG
TGACCGTCTCCGGGAGCTGCATGTGTCAGAGGTTTTCACCGTCAT
CACCGAAACGCGCGAGGCAGCTGCGGTAAAGCTCATCAGCGTGGT
CGTGAAGCGATTCACAGATGTCTGCCTGTTCATCCGCGTCCAGCT
CGTTGAGTTTCTCCAGAAGCGTTAATGTCTGGCTTCTGATAAAGC
GGGCCATGTTAAGGGCGGTTTTTTCCTGTTTGGTCACTGATGCCT
CCGTGTAAGGGGGATTTCTGTTCATGGGGGTAATGATACCGATGA
AACGAGAGAGGATGCTCACGATACGGGTTACTGATGATGAACAAG
AGGACATCCGGTAAGAGGACATCCGGTCAAATAAAACGAAAGGCT
CAGTCGAAAGACTGGGCCTTTCGTTTTGCTGAGGAGACTTAGGGA
CTCTCACAGCTAACACCACGTCGTCCCTATCTGCTGCCCTAGGTC
TATGAGTGGTTGCTGGATAACTTTACGGGCATGCATAAGGCTCGT
ATAATATATTCAGGGAGACCACAACGGTTTCCCTCTACAAATAAT
TTTGTTTAACTTTTACTAGAGTGGGACCCTACCTGCAGGTGCAGT
AAAGGAAAAAAAAAATGAAAAAATTATTATTCGCAATTCCTTTAT
GTCTCAGCTACGAAACTGAAATCTTGACCGTCGAATATGGTCTGC
TGCCAATCGGCAAGATTGTTGAAAAACGTATTGAATGTACGGTCT
ACTCAGTGGATAACAACGGCAATATCTACACCCAGCCGGTGGCCC
AGTGGCATGACCGTGGTGAACAGGAAGTGTTCGAATATTGTCTGG
AAGACGGATCTTTAATCCGTGCCACAAAGGATCACAAATTTATGA
CTGTAGATGGTCAGATGCTCCCAATCGACGAAATTTTTGAACGCG
AATTAGACCTGATGCGCGTGGATAATCTCCCGAATTCCGGAGGAT
CTAGCGGGGNRNCCTCTGGCTCTGAGTAGTGAGGAACAAGCGAGA
GTGCAACACCTGAAAGCAGCGGGGGCAGCAGCGGGGGGTCATCTG
GAGGAAGTTCGGGAGGAAGTTCTGGCAGCGAGACACCAGGAACAA
GCGAGTCAGCAACACCAGAGAGCAGTGGCGGCTCGAGCGGTGGTA
GCTCTGGTGGGTCAAAAAGAACCGCCGACGGCAGCGAATTCGAGC
CCAAGAAGAAGAGGAAAGTGGGATCCTACCCATACGATGTTCCAG
ATTACGCTTATCCCTACGACGTGCCTGATTATGCATACCCATATG
ATGTCCCCGACTATGCCGGGGGGTCAAGCGGGGGGTCAATCAAAA
TTGCCACGCGTAAATATTTAGGCAAACAGAATGTTTATGATATCG
GTGTCGAGCGCGATCATAATTTCGCGCTGAAAAACGGATTTATAG
CTAGCAATTGTTTCAACGTTGTTCCTTTCTATTCTCACTCCGCTG
AAACTGTTGAAAGTTGTTTAGCAAAACCCCATACAGAAAATTCAT
TTACTAACGTCTGGAAAGACGACAAAACTTTAGATCGTTACGCTA
ACTATGAGGGCTGTCTGTGGAATGCTACAGGCGTTGTAGTTTGTA
CTGGTGACGAAACTCAGTGTTACGGTACATGGGTTCCTATTGGGC
TTGCTATCCCTGAAAATGAGGGTGGTGGCTCTGAGGGTGGCGGTT
CTGAGGGTGGCGGTTCTGAGGGTGGCGGTACTAAACCTCCTGAGT
ACGGTGATACACCTATTCCGGGCTATACTTATATCAACCCTCTCG
ACGGCACTTATCCGCCTGGTACTGAGCAAAACCCCGCTAATCCTA
ATCCTTCTCTTGAGGAGTCTCAGCCTCTTAATACTTTCATGTTTC
AGAATAATAGGTTCCGAAATAGGCAGGGGGCATTAACTGTTTATA
CGGGCACTGTTACTCAAGGCACTGACCCCGTTAAAACTTATTACC
AGTACACTGCTGTATCATCAAAAGCCATGTATGACGCTTACTGGA
ACGGTAAATTCAGAGACTGCGCTTTCCATTCTGGCTTTAATGAGG
ATCCATTCGTTTGTGAATATCAAGGCCAATCGTCTGACCTGCCTC
AACCTCCTGTCAATGCTGGCGGCGGCTCTGGTGGTGGTTCTGGTG
GCGGCTCTGAGGGTGGTGGCTCTGAGGGTGGCGGTTCTGAGGGTG
GCGGCTCTGAGGGAGGCGGTTCCGGTGGTGGCTCTGGTTCCGGTG
ATTTTGATTATGAAAAGATGGCAAACGCTAATAAGGGGGCTATGA
CCGAAAATGCCGATGAAAACGCGCTACAGTCTGACGCTAAAGGCA
AACTTGATTCTGTCGCTACTGATTACGGTGCTGCTATCGATGGTT
TCATTGGTGACGTTTCCGGCCTTGCTAATGGTAATGGTGCTACTG
GTGATTTTGCTGGCTCTAATTCCCAAATGGCTCAAGTCGGTGACG
GTGATAATTCACCTTTAATGAATAATTTCCGTCAATATTTACCTT
CCCTCCCTCAATCGGTTGAATGTCGCCCTTTTGTCTTTGGCGCTG
GTAAACCTTACGAGTTCAGTATCGACTGCGATAAGATCAACCTGT
TCCGCGGTGTCTTTGCGTTTCTTTTATATGTTGCCACCTTTATGT
ATGTATTTTCTACGTTTGCTAACATACTGCGTAATAAGGAGTCTT
AATGAAATTTGGAAACTTTTTGCTTACATACCAACCTCCCCAATT
TTCCCAAACAGAGGTAATGAAACGTTTGGTTAAATTAGGTCGCAT
CTCTGAGGAGTGTGGTTTTGATACCGTATGGTTACTGGAGCATCA
TTTCACGGAGTTTGGTTTGCTTGGTAACCCTTATGTCGCTGCTGC
ATATTTACTTGGCGCGACTAAAAAATTGAATGTAGGAACTGCCGC
TATTGTTCTTCCCACAGCCCATCCAGTACGCCAACTTGAAGATGT
GAATTTATTGGATCAAATGTCAAAAGGACGATTTCGGTTTGGTAT
TTGCCGAGGGCTTTACAACAAGGACTTTCGCGTATTCGGCACAGA
TATGAATAACAGTCGCGCCTTAGCGGAATGCTGGTACGGGCTGAT
AAAGAATGGCATGACAGAGGGATATATGGAAGCTGATAATGAACA
TATCAAGTTCCATAAGGTAAAAGTAAACCCCGCGGCGTATAGCAG
AGGTGGCGCACCGGTTTATGTGGTGGCTGAATCAGCTTCGACGAC
TGAGTGGGCTGCTCAATTTGGCCTACCGATGATATTAAGTTGGAT
TATAAATACTAACGAAAAGAAAGCACAACTTGAGCTTTATAATGA
AGTGGCTCAAGAATATGGGCACGATATTCATAATATCGACCATTG
CTTATCATATATAACATCTGTAGATCATGACTCAATTAAAGCGAA
AGAGATTTGCCGGAAATTTCTGGGGCATTGGTATGATTCTTATGT
GAATGCTACGACTATTTTTGATGATTCAGACCAAACAAGAGGTTA
TGATTTCAATAAAGGGCAGTGGCGTGACTTTGTATTAAAAGGACA
TAAAGATACTAATCGCCGTATTGATTACAGTTACGAAATCAATCC
CGTGGGAACGCCGCAGGAATGTATTGACATAATTCAAAAAGACAT
TGATGCTACAGGAATATCAAATATTTGTTGTGGATTTGAAGCTAA
TGGAACAGTAGACGAAATTATTGCTTCCATGAAGCTCTTCCAGTC
TGATGTCATGCCATTTCTTAAAGAAAAACAACGTTCGCTATTATA
TTATGGCGGTGGCGGTAGCGGCGGTGGCGGTAGCGGCGGTGGCGG
TAGCGGCGGTGGCGGTAGCAAATTTGGATTGTTCTTCCTTAACTT
CATCAATTCAACAACTGTTCAAGAACAGAGTATAGTTCGCATGCA
GGAAATAACGGAGTATGTTGATAAGTTGAATTTTGAACAGATTTT
AGTGTATGAAAATCATTTTTCAGATAATGGTGTTGTCGGCGCTCC
TCTGACTGTTTCTGGTTTTCTGCTCGGTTTAACAGAGAAAATTAA
AATTGGTTCATTAAATCACATCATTACAACTCATCATCCTGTCCG
CATAGCGGAGGAAGCTTGCTTATTGGATCAGTTAAGTGAAGGGAG
ATTTATTTTAGGGTTTAGTGATTGCGAAAAAAAAGATGAAATGCA
TTTTTTTAATCGCCCGGTTGAATATCAACAGCAACTATTTGAAGA
GTGTTATGAAATCATTAACGATGCTTTAACAACAGGCTATTGTAA
TCCAGATAACGATTTTTATAGCTTCCCTAAAATATCTGTAAATCC
CCATGCTTATACGCCAGGCGGACCTCGGAAATATGTAACAGCAAC
CAGTCATCATATTGTTGAGTGGGCGGCCAAAAAAGGTATTCCTCT
CATCTTTAAGTGGGATGATTCTAATGATGTTAGATATGAATATGC
TGAAAGATATAAAGCCGTTGCGGATAAATATGACGTTGACCTATC
AGAGATAGACCATCAGTTAATGATATTAGTTAACTATAACGAAGA
TAGTAATAAAGCTAAACAAGAGACGCGTGCATTTATTAGTGATTA
TGTTCTTGAAATGCACCCTAATGAAAATTTCGAAAATAAACTTGA
AGAAATAATTGCAGAAAACGCTGTCGGAAATTATACGGAGTGTAT
AACTGCGGCTAAGTTGGCAATTGAAAAGTGTGGTGCGAAAAGTGT
ATTGCTGTCCTTTGAACCAATGAATGATTTGATGAGCCAAAAAAA
TGTAATCAATATTGTTGATGATAATATTAAGAAGTACCACACGGA
ATATACCTAAACGGAGCCAATGTACGCAAAAAACCCCGCTTCAGC
GGGGTTTTTTCGCCAAAAAAAACCCCGCCCTGTCAGGGGCGGGGT
TTTTTTTTAGGGCCCTACTGACTGTAAAAAAATAAACGATGCCCC
TTAAAGCAGAAGCTTTAAGGGGCAGAGCGTTGCGGCACATCTTTT
CAGACGGCCTTATTGTAGCAACGGTTCTCATTTCGTTTCCGAAAT
GAGAAAGGGAGCTACAACCTGGCTCTGAGTAGTGAGGAACACCAC
ACAACATACGAGCCGGAAGCATAAAGTGTAAAGCCTGGGGTGCCT
AATGAGTGAGCTAACTCACATTAATTGCGTTGCGCTCACTGCCCG
CTTTCCAGTCGGGAAACCTGTCGTGCCAGCTGCATTAATGAATCG
GCCAACGCGCGGGGAGAGGCGGTTTGCGTATTGGGCGCACTTAAT
TAACGGCACTGCGAAAAAACCCCGCCGAAGCGGGGTTTTTTGCGA
AATATAATGACCCTCTTGATAACCCAAGAGGGCATTTTTTAATGC
CCATGGCGTTTACCAATGCTTAATCAGTGAGGCACCTATCTCAGC
GATCTGTCTATTTCGTTCATCCATAGTTGCCTGACTCCCCGTCGT
GTAGATAACTACGATACGGGAGGGCTTACCATCTGGCCCCAGTGC
TGCAATGATACCGCGAGACCCACGCTCACCGGCTCCAGATTTATC
AGCAATAAACCAGCCAGCCGGAAGGGCCGAGCGCAGAAGTGGTCC
TGCAACTTTATCCGCCTCCATCCAGTCTATTAATTGTTGCCGGGA
AGCTAGAGTAAGTAGTTCGCCAGTTAATAGTTTGCGCAACGTTGT
TGCCATTGCTACAGGCATCGTGGTGTCACGCTCGTCGTTTGGTAT
GGCTTCATTCAGCTCCGGTTCCCAACGATCAAGGCGAGTTACATG
ATCCCCCATGTTGTGCAAAAAAGCGGTTAGCTCCTTCGGTCCTCC
GATCGTTGTCAGAAGTAAGTTGGCCGCAGTGTTATCACTCATGGT
TATGGCAGCACTGCATAATTCTCTTACTGTCATGCCATCCGTAAG
ATGCTTTTCTGTGACTGGTGAGTACTCAACCAAGTCATTCTGAGA
ATAGTGTATGCGGCGACCGAGTTGCTCTTGCCCGGCGTCAATACG
GGATAATACCGCGCCACATAGCAGAACTTTAAAAGTGCTCATCAT
TGGAAAACGTTCTTCGGGGCGAAAACTCTCAAGGATCTTACCGCT
GTTGAGATCCAGTTCGATGTAACCCACTCGTGCACCCAACTGATC
TTCAGCATCTTTTACTTTCACCAGCGTTTCTGGGTGAGCAAAAAC
AGGAAGGCAAAATGCCGCAAAAAAGGGAATAAGGGCGACACGGAA
ATGTTGAATACTCATACTCTTCCTTTTTCAATATTATTGAAGCAT
TTATCAGGGTTATTGTCTCATGAGCGGATACATATTTGAATGTAT
TTAGAAAAATAAACAAATAGGGGTTCCGCGCACATTTCCCCGAAA
AGTGCCACCTGACGTCTAAGAAACCATTATTATCATGACATTAAC
CTATAAAAATAGGCGTATCACGAGGCCCTTAGGCCAAATAGGCCG
T
pTPH405
(SEQ ID NO: 8)
TGAGATCCTTTTTTTCTGCGCGTAATCTGCTGCTTGCAAACAAAA
AAACCACCGCTACCAGCGGTGGTTTGTTTGCCGGATCAAGAGCTA
CCACCTCTTTTTCCGAAGGTAACTGGCTTCAGCAGAGCGCAGATA
CCAAATACTGTCCTTCTAGTGTAGCCGTAGTTAGGCCACCACTTC
AAGAACTCTGTAGCACCGCCTACATACCTCGCTCTGCTAATCCTG
TTACCAGTGGCTGCTGCCAGTGGCGATAAGTCGTGTCTTACCGGG
TTGGACTCAAGACGATAGTTACCGGATAAGGCGCAGCGGTCGGGC
TGAACGGGGGGTTCGTGCACACAGCCCAGCTTGGAGCGAACGACC
TACACCGAACTGAGATACCTACAGCGTGAGCTATGAGAAAGCGCC
ACGCTTCCCGAAGGGAGAAAGGCGGACAGGTATCCGGTAAGCGGC
AGGGTCGGAACAGGAGAGCGCACGAGGGAGCTTCCAGGGGGAAAC
GCCTGGTATCTTTATAGTCCTGTCGGGTTTCGCCACCTCTGACTT
GAGCGTCGATTTTTGTGATGCTCGTCAGGGGGGCGGAGCCTATGG
AAAAACGCCAGCAACGCGGCCTTTTTACGGTTCCTGGCCTTTTGC
TGGCCTTTTGCTCACATGTTCTTTCCTGCGTTATCCCCTGATTCT
GTGGATAACCGTATTACCGCCTTTGAGTGAGCTGATACCGCTCGC
CGCAGCCGAACGACCGAGCGCAGCGAGTCAGTGAGCGAGGAAGCG
GAAGAGCGCCTGATGCGGTATTTTCTCCTTACGCATCTGTGCGGT
ATTTCACACCGCATATGGTGCACTCTCAGTACAATCTGCTCTGAT
GCCCCATAGTTAAGCCAGTATACACTCCGCTATCGCTACGTGACT
GGGTCATGGCTGCGCCCCGACACCCGCCAACACCCGCTGACGCGC
CCTGACGGGCTTGTCTGCTCCCGGCATCCGCTTACAGACAAGCTG
TGACCGTCTCCGGGAGCTGCATGTGTCAGAGGTTTTCACCGTCAT
CACCGAAACGCGCGAGGCAGCTGCGGTAAAGCTCATCAGCGTGGT
CGTGAAGCGATTCACAGATGTCTGCCTGTTCATCCGCGTCCAGCT
CGTTGAGTTTCTCCAGAAGCGTTAATGTCTGGCTTCTGATAAAGC
GGGCCATGTTAAGGGCGGTTTTTTCCTGTTTGGTCACTGATGCCT
CCGTGTAAGGGGGATTTCTGTTCATGGGGGTAATGATACCGATGA
AACGAGAGAGGATGCTCACGATACGGGTTACTGATGATGAACAAG
AGGACATCCGGTAAGAGGACATCCGGTCAAATAAAACGAAAGGCT
CAGTCGAAAGACTGGGCCTTTCGTTTTGCTGAGGAGACTTAGGGA
CTCTCACAGCTAACACCACGTCGTCCCTATCTGCTGCCCTAGGTC
TATGAGTGGTTGCTGGATAACTTTACGGGCATGCATAAGGCTCGT
ATAATATATTCAGGGAGACCACAACGGTTTCCCTCTACAAATAAT
TTTGTTTAACTTTTACTAGAGTGGGACCCTACCTGCAGGTGCAGT
AAAGGAAAAAAAAAATGAAAAAATTATTATTTGCGATACCGCTGT
GCTTAAGCTACGAAACTGAAATCTTGACCGTCGAATATGGTCTGC
TGCCAATCGGCAAGATTGTTGAAAAACGTATTGAATGTACGGTCT
ACTCAGTGGATAACAACGGCAATATCTACACCCAGCCGGTGGCCC
AGTGGCATGACCGTGGTGAACAGGAAGTGTTCGAATATTGTCTGG
AAGACGGATCTTTAATCCGTGCCACAAAGGATCACAAATTTATGA
CTGTAGATGGTCAGATGCTCCCAATCGACGAAATTTTTGAACGCG
AATTAGACCTGATGCGCGTGGATAATCTCCCGAATTCCGGAGGAT
CTAGCGGGGCAACCTCTGGCTCTGAGTAGTGAGGAACAAGCGAGA
GTGCAACACCTGAAAGCAGCGGGGGCAGCAGCGGGGGGTCAATCA
AAATTGCCACGCGTAAATATTTAGGCAAACAGAATGTTTATGATA
TCGGTGTCGAGCGCGATCATAATTTCGCGCTGAAAAACGGATTTA
TAGCTAGCAATTGTTTCAACGTTGTTCCTTTCTATTCTCACTCCG
CTGAAACTGTTGAAAGTTGTTTAGCAAAACCCCATACAGAAAATT
CATTTACTAACGTCTGGAAAGACGACAAAACTTTAGATCGTTACG
CTAACTATGAGGGCTGTCTGTGGAATGCTACAGGCGTTGTAGTTT
GTACTGGTGACGAAACTCAGTGTTACGGTACATGGGTTCCTATTG
GGCTTGCTATCCCTGAAAATGAGGGTGGTGGCTCTGAGGGTGGCG
GTTCTGAGGGTGGCGGTTCTGAGGGTGGCGGTACTAAACCTCCTG
AGTACGGTGATACACCTATTCCGGGCTATACTTATATCAACCCTC
TCGACGGCACTTATCCGCCTGGTACTGAGCAAAACCCCGCTAATC
CTAATCCTTCTCTTGAGGAGTCTCAGCCTCTTAATACTTTCATGT
TTCAGAATAATAGGTTCCGAAATAGGCAGGGGGCATTAACTGTTT
ATACGGGCACTGTTACTCAAGGCACTGACCCCGTTAAAACTTATT
ACCAGTACACTCCTGTATCATCAAAAGCCATGTATGACGCTTACT
GGAACGGTAAATTCAGAGACTGCGCTTTCCATTCTGGCTTTAATG
AGGATCCATTCGTTTGTGAATATCAAGGCCAATCGTCTGACCTGC
CTCAACCTCCTGTCAATGCTGGCGGCGGCTCTGGTGGTGGTTCTG
GTGGCGGCTCTGAGGGTGGTGGCTCTGAGGGTGGCGGTTCTGAGG
GTGGCGGCTCTGAGGGAGGCGGTTCCGGTGGTGGCTCTGGTTCCG
GTGATTTTGATTATGAAAAGATGGCAAACGCTAATAAGGGGGCTA
TGACCGAAAATGCCGATGAAAACGCGCTACAGTCTGACGCTAAAG
GCAAACTTGATTCTGTCGCTACTGATTACGGTGCTGCTATCGATG
GTTTCATTGGTGACGTTTCCGGCCTTGCTAATGGTAATGGTGCTA
CTGGTGATTTTGCTGGCTCTAATTCCCAAATGGCTCAAGTCGGTG
ACGGTGATAATTCACCTTTAATGAATAATTTCCGTCAATATTTAC
CTTCCCTCCCTCAATCGGTTGAATGTCGCCCTTTTGTCTTTGGCG
CTGGTAAACCTTACGAGTTCAGTATCGACTGCGATAAGATCAACC
TGTTCCGCGGTGTCTTTGCGTTTCTTTTATATGTTGCCACCTTTA
TGTATGTATTTTCTACGTTTGCTAACATACTGCGTAATAAGGAGT
CTTAATGAAATTTGGAAACTTTTTGCTTACATACCAACCTCCCCA
ATTTTCCCAAACAGAGGTAATGAAACGTTTGGTTAAATTAGGTCG
CATCTCTGAGGAGTGTGGTTTTGATACCGTATGGTTACTGGAGCA
TCATTTCACGGAGTTTGGTTTGCTTGGTAACCCTTATGTCGCTGC
TGCATATTTACTTGGCGCGACTAAAAAATTGAATGTAGGAACTGC
CGCTATTGTTCTTCCCACAGCCCATCCAGTACGCCAACTTGAAGA
TGTGAATTTATTGGATCAAATGTCAAAAGGACGATTTCGGTTTGG
TATTTGCCGAGGGCTTTACAACAAGGACTTTCGCGTATTCGGCAC
AGATATGAATAACAGTCGCGCCTTAGCGGAATGCTGGTACGGGCT
GATAAAGAATGGCATGACAGAGGGATATATGGAAGCTGATAATGA
ACATATCAAGTTCCATAAGGTAAAAGTAAACCCCGCGGCGTATAG
CAGAGGTGGCGCACCGGTTTATGTGGTGGCTGAATCAGCTTCGAC
GACTGAGTGGGCTGCTCAATTTGGCCTACCGATGATATTAAGTTG
GATTATAAATACTAACGAAAAGAAAGCACAACTTGAGCTTTATAA
TGAAGTGGCTCAAGAATATGGGCACGATATTCATAATATCGACCA
TTGCTTATCATATATAACATCTGTAGATCATGACTCAATTAAAGC
GAAAGAGATTTGCCGGAAATTTCTGGGGCATTGGTATGATTCTTA
TGTGAATGCTACGACTATTTTTGATGATTCAGACCAAACAAGAGG
TTATGATTTCAATAAAGGGCAGTGGCGTGACTTTGTATTAAAAGG
ACATAAAGATACTAATCGCCGTATTGATTACAGTTACGAAATCAA
TCCCGTGGGAACGCCGCAGGAATGTATTGACATAATTCAAAAAGA
CATTGATGCTACAGGAATATCAAATATTTGTTGTGGATTTGAAGC
TAATGGAACAGTAGACGAAATTATTGCTTCCATGAAGCTCTTCCA
GTCTGATGTCATGCCATTTCTTAAAGAAAAACAACGTTCGCTATT
ATATTATGGCGGTGGCGGTAGCGGCGGTGGCGGTAGCGGCGGTGG
CGGTAGCGGCGGTGGCGGTAGCAAATTTGGATTGTTCTTCCTTAA
CTTCATCAATTCAACAACTGTTCAAGAACAGAGTATAGTTCGCAT
GCAGGAAATAACGGAGTATGTTGATAAGTTGAATTTTGAACAGAT
TTTAGTGTATGAAAATCATTTTTCAGATAATGGTGTTGTCGGCGC
TCCTCTGACTGTTTCTGGTTTTCTGCTCGGTTTAACAGAGAAAAT
TAAAATTGGTTCATTAAATCACATCATTACAACTCATCATCCTGT
CCGCATAGCGGAGGAAGCTTGCTTATTGGATCAGTTAAGTGAAGG
GAGATTTATTTTAGGGTTTAGTGATTGCGAAAAAAAAGATGAAAT
GCATTTTTTTAATCGCCCGGTTGAATATCAACAGCAACTATTTGA
AGAGTGTTATGAAATCATTAACGATGCTTTAACAACAGGCTATTG
TAATCCAGATAACGATTTTTATAGCTTCCCTAAAATATCTGTAAA
TCCCCATGCTTATACGCCAGGCGGACCTCGGAAATATGTAACAGC
AACCAGTCATCATATTGTTGAGTGGGCGGCCAAAAAAGGTATTCC
TCTCATCTTTAAGTGGGATGATTCTAATGATGTTAGATATGAATA
TGCTGAAAGATATAAAGCCGTTGCGGATAAATATGACGTTGACCT
ATCAGAGATAGACCATCAGTTAATGATATTAGTTAACTATAACGA
AGATAGTAATAAAGCTAAACAAGAGACGCGTGCATTTATTAGTGA
TTATGTTCTTGAAATGCACCCTAATGAAAATTTCGAAAAAAACTT
GAAGAAATAATTGCAGAAAACGCTGTCGGAAATTATACGGAGTGT
ATAACTGCGGCTAAGTTGGCAATTGAAAAGTGTGGTGCGAAAAGT
GTATTGCTGTCCTTTGAACCAATGAATGATTTGATGAGCCAAAAA
AATGTAATCAATATTGTTGATGATAATATTAAGAAGTACCACACG
GAATATACCTAAACGGAGCCAATGTACGCAAAAAACCCCGCTTCA
GCGGGGTTTTTTCGCCAAAAAAAACCCCGCCCTGTCAGGGGCGGG
GTTTTTTTTTAGGGCCCTACTGACTGTAAAAAAATAAACGATGCC
CCTTAAAGCAGAAGCTTTAAGGGGCAGAGCGTTGCGGCACATCTT
TTCAGACGGCCTTATTGTAGCAACGGTTCTCATTTCGTTTCCGAA
ATGAGAAAGGGAGCTACAACCTGGCTCTGAGTAGTGAGGAACACC
ACACAACATACGAGCCGGAAGCATAAAGTGTAAAGCCTGGGGTGC
CTAATGAGTGAGCTAACTCACATTAATTGCGTTGCGCTCACTGCC
CGCTTTCCAGTCGGGAAACCTGTCGTGCCAGCTGCATTAATGAAT
CGGCCAACGCGCGGGGAGAGGCGGTTTGCGTATTGGGCGCACTTA
ATTAACGGCACTGCGAAAAAACCCCGCCGAAGCGGGGTTTTTTGC
GAAATATAATGACCCTCTTGATAACCCAAGAGGGCATTTTTTAAT
GCCCATGGCGTTTACCAATGCTTAATCAGTGAGGCACCTATCTCA
GCGATCTGTCTATTTCGTTCATCCATAGTTGCCTGACTCCCCGTC
GTGTAGATAACTACGATACGGGAGGGCTTACCATCTGGCCCCAGT
GCTGCAATGATACCGCGAGACCCACGCTCACCGGCTCCAGATTTA
TCAGCAATAAACCAGCCAGCCGGAAGGGCCGAGCGCAGAAGTGGT
CCTGCAACTTTATCCGCCTCCATCCAGTCTATTAATTGTTGCCGG
GAAGCTAGAGTAAGTAGTTCGCCAGTTAATAGTTTGCGCAACGTT
GTTGCCATTGCTACAGGCATCGTGGTGTCACGCTCGTCGTTTGGT
ATGGCTTCATTCAGCTCCGGTTCCCAACGATCAAGGCGAGTTACA
TGATCCCCCATGTTGTGCAAAAAAGCGGTTAGCTCCTTCGGTCCT
CCGATCGTTGTCAGAAGTAAGTTGGCCGCAGTGTTATCACTCATG
GTTATGGCAGCACTGCATAATTCTCTTACTGTCATGCCATCCGTA
AGATGCTTTTCTGTGACTGGTGAGTACTCAACCAAGTCATTCTGA
GAATAGTGTATGCGGCGACCGAGTTGCTCTTGCCCGGCGTCAATA
CGGGATAATACCGCGCCACATAGCAGAACTTTAAAAGTGCTCATC
ATTGGAAAACGTTCTTCGGGGCGAAAACTCTCAAGGATCTTACCG
CTGTTGAGATCCAGTTCGATGTAACCCACTCGTGCACCCAACTGA
TCTTCAGCATCTTTTACTTTCACCAGCGTTTCTGGGTGAGCAAAA
ACAGGAAGGCAAAATGCCGCAAAAAAGGGAATAAGGGCGACACGG
AAATGTTGAATACTCATACTCTTCCTTTTTCAATATTATTGAAGC
ATTTATCAGGGTTATTGTCTCATGAGCGGATACATATTTGAATGT
ATTTAGAAAAATAAACAAATAGGGGTTCCGCGCACATTTCCCCGA
AAAGTGCCACCTGACGTCTAAGAAACCATTATTATCATGACATTA
ACCTATAAAAATAGGCGTATCACGAGGCCCTTAGGCCAAATAGGC
CGT
pTPH405c
(SEQ ID NO: 9)
TCAGATCCTTCCGTATTTAGCCAGTATGTTCTCTAGTGTGGTTCG
TTGTTTTTGCGTGAGCCATGAGAACGAACCATTGAGATCATGCTT
ACTTTGCATGTCACTCAAAAATTTTGCCTCAAAACTGGTGAGCTG
AATTTTTGCAGTTAAAGCATCGTGTAGTGTTTTTCTTAGTCCGTT
ACGTAGGTAGGAATCTGATGTAATGGTTGTTGGTATTTTGTCACC
ATTCATTTTTATCTGGTTGTTCTCAAGTTCGGTTACGAGATCCAT
TTGTCTATCTAGTTCAACTTGGAAAATCAACGTATCAGTCGGGCG
GCCTCGCTTATCAACCACCAATTTCATATTGCTGTAAGTGTTTAA
ATCTTTACTTATTGGTTTCAAAACCCATTGGTTAAGCCTTTTAAA
CTCATGGTAGTTATTTTCAAGCATTAACATGAACTTAAATTCATC
AAGGCTAATCTCTATATTTGCCTTGTGAGTTTTCTTTTGTGTTAG
TTCTTTTAATAACCACTCATAAATCCTCATAGAGTATTTGTTTTC
AAAAGACTTAACATGTTCCAGATTATATTTTATGAATTTTTTTAA
CTGGAAAAGATAAGGCAATATCTCTTCACTAAAAACTAATTCTAA
TTTTTCGCTTGAGAACTTGGCATAGTTTGTCCACTGGAAAATCTC
AAAGCCTTTAACCAAAGGATTCCTGATTTCCACAGTTCTCGTCAT
CAGCTCTCTGGTTGCTTTAGCTAATACACCATAAGCATTTTCCCT
ACTGATGTTCATCATCTGAGCGTATTGGTTATAAGTGAACGATAC
CGTCCGTTCTTTCCTTGTAGGGTTTTCAATCGTGGGGTTGAGTAG
TGCCACACAGCATAAAATTAGCTTGGTTTCATGCTCCGTTAAGTC
ATAGCGACTAATCGCTAGTTCATTTGCTTTGAAAACAACTAATTC
AGACATACATCTCAATTGGTCTAGGTGATTTTAATCACTATACCA
ATTGAGATGGGCTAGTCAATGATAATTACTAGTCCTTTTCCTTTG
AGTTGTGGGTATCTGTAAATTCTGCTAGACCTTTGCTGGAAAACT
TGTAAATTCTGCTAGACCCTCTGTAAATTCCGCTAGACCTTTGTG
TGTTTTTTTTGTTTATATTCAAGTGGTTATAATTTATAGAATAAA
GAAAGAATAAAAAAAGATAAAAAGAATAGATCCCAGCCCTGTGTA
TAACTCACTACTTTAGTCAGTTCCGCAGTATTACAAAAGGATGTC
GCAAACGCTGTTTGCTCCTCTACAAAACAGACCTTAAAACCCTAA
AGGCTTAAGTAGCACCCTCGCAAGCTCGGGCAAATCGCTGAATAT
TCCTTTTGTCTCCGACCATCAGGCACCTGAGTCGCTGTCTTTTTC
GTGACATTCAGTTCGCTGCGCTCACGGCTCTGGCAGTGAATGGGG
GTAAATGGCACTACAGGCGCCTTTTATGGATTCATGCAAGGAAAC
TACCCATAATACAAGAAAAGCCCGTCACGGGCTTCTCAGGGCGTT
TTATGGCGGGTCTGCTATGTGGTGCTATCTGACTTTTTGCTGTTC
AGCAGTTCCTGCCCTCTGATTTTCCAGTCTGACCACTTCGGATTA
TCCCGTGACAGGTCATTCAGACTGGCTAATGCACCCAGTAAGGCA
GCGGTATCATCAACAGGCTTACCCGTCTTACTGTCAAGAGGACAT
CCGGTCAAATAAAACGAAAGGCTCAGTCGAAAGACTGGGCCTTTC
GTTTTGCTGAGGAGACTTAGGGACCCTGATGAAATTCGCCACTTG
TTAGTGTAATTCGCTAACTCATCCTGGCATGTTGCTGTTGATTCT
TCAATCAGATCTTTATAAATCAAAAAGATAAAAAATTGGCACGCA
AATTGTATTAACAGTTCAGCAGGACAATCCTGAACGCAGAAATCA
AGAGGACAACATTACCTGCAGGTGCAGTAAAGGAAAAAAAAAATG
TCTGAGGTGGAGTTTTCCCACGAGTACTGGATGAGACATGCCCTG
ACCCTGGCCAAGAGGGCACGGGATGAGAGGGAGGTGCCTGTGGGA
GCCGTGCTGGTGCTGAACAATAGAGTGATCGGCGAGGGCTGGAAC
AGAGCCATCGGCCTGCACGACCCAACAGCCCATGCCGAAATTATG
GCCCTGAGACAGGGCGGCCTGGTCATGCAGAACTACAGACTGATT
GACGCCACCCTGTACGTGACATTCGAGCCTTGCGTGATGTGCGCC
GGCGCCATGATCCACTCTAGGATCGGCCGCGTGGTGTTTGGCGTG
AGAAACTCAAAAAGAGGCGCCGCAGGCTCCCTGATGAACGTGCTG
AACTACCCCGGCATGAATCACCGCGTCGAAATTACCGAGGGAATC
CTGGCAGATGAATGTGCCGCCCTGCTGTGCGATTTCTATCGGATG
CCTAGACAGGTGTTCAATGCTCAGAAGAAGGCCCAGAGCTCCATC
AACAAGGAATCTGGCTCTGTATCTTCTGAACAGTTGGCGCAATTC
CGCTCTCTGGACAGCCAGCTGAACCGCTGCCTGAGCCTGGATACC
ATGGTGGTGACCAACGGCAAAGCGATTGAAATTCGCGATGTGAAA
GTGGGCGATTGGCTGGAAAGCGAATGCGGCCCGGTGCAGGTGACC
GAAGTGCTGCCGATTATTAAACAGCCGGTGTTTGAAATTGTGCTG
AAAAGCGGCAAAAAAATTCGCGTGAGCGCGAACCATAAATTTCCG
ACCAAAGATGGCCTGAAAACCATTAACAGCGGCCTGAAAGTGGGC
GATTTTCTGCGCAGCCGCGCGAAATAACTTAATTAACGGCACTCC
TCAGCAAATATAATGACCCTCTTGATAACCCAAGAGGGCATTTTT
TAATGCCCATGGCGTTTAGAAAAACTCATCGAGCATCAAATGAAA
CTGCAATTTATTCATATCAGGATTATCAATACCATATTTTTGAAA
AAGCCGTTTCTGTAATGAAGGAGAAAACTCACCGAGGCAGTTCCA
TAGGATGGCAAGATCCTGGTATCGGTCTGCGATTCCGACTCGTCC
AACATCAATACAACCTATTAATTTCCCCTCGTCAAAAATAAGGTT
ATCAAGTGAGAAATCACCATGAGTGACGACTGAATCCGGTGAGAA
TGGCAAAAGCTTATGCATTTCTTTCCAGACTTGTTCAACAGGCCA
GCCATTACGCTCGTCATCAAAATCACTCGCATCAACCAAACCGTT
ATTCATTCGTGATTGCGCCTGAGCGAGACGAAATACGCGATCGCT
GTTAAAAGGACAATTACAAACAGGAATCGAATGCAACCGGCGCAG
GAACACTGCCAGCGCATCAACAATATTTTCACCTGAATCAGGATA
TTCTTCTAATACCTGGAATGCTGTTTTCCCGGGGATCGCAGTGGT
GAGTAACCATGCATCATCAGGAGTACGGATAAAATGCTTGATGGT
CGGAAGAGGCATAAATTCCGTCAGCCAGTTTAGTCTGACCATCTC
ATCTGTAACATCATTGGCAACGCTACCTTTGCCATGTTTCAGAAA
CAACTCTGGCGCATCGGGCTTCCCATACAATCGATAGATTGTCGC
ACCTGATTGCCCGACATTATCGCGAGCCCATTTATACCCATATAA
ATCAGCATCCATGTTGGAATTTAATCGCGGCCTCGAGCAAGACGT
TTCCCGTTGAATATGGCTCATAACACCCCTTGTATTACTGTTTAT
GTAAGCAGACAGTTTTATTGTTCATGATGATATATTTTTATCTTG
TGCAATGTAACATCAGAGATTTTGAAGGCCAAATAGGCCGT
pTPH412
(SEQ ID NO: 10)
TGAGATCCTTTTTTTCTGCGCGTAATCTGCTGCTTGCAAACAAAA
AAACCACCGCTACCAGCGGTGGTTTGTTTGCCGGATCAAGAGCTA
CCACCTCTTTTTCCGAAGGTAACTGGCTTCAGCAGAGCGCAGATA
CCAAATACTGTCCTTCTAGTGTAGCCGTAGTTAGGCCACCACTTC
AAGAACTCTGTAGCACCGCCTACATACCTCGCTCTGCTAATCCTG
TTACCAGTGGCTGCTGCCAGTGGCGATAAGTCGTGTCTTACCGGG
TTGGACTCAAGACGATAGTTACCGGATAAGGCGCAGCGGTCGGGC
TGAACGGGGGGTTCGTGCACACAGCCCAGCTTGGAGCGAACGACC
TACACCGAACTGAGATACCTACAGCGTGAGCTATGAGAAAGCGCC
ACGCTTCCCGAAGGGAGAAAGGCGGACAGGTATCCGGTAAGCGGC
AGGGTCGGAACAGGAGAGCGCACGAGGGAGCTTCCAGGGGGAAAC
GCCTGGTATCTTTATAGTCCTGTCGGGTTTCGCCACCTCTGACTT
GAGCGTCGATTTTTGTGATGCTCGTCAGGGGGGCGGAGCCTATGG
AAAAACGCCAGCAACGCGGCCTTTTTACGGTTCCTGGCCTTTTGC
TGGCCTTTTGCTCACATGTTCTTTCCTGCGTTATCCCCTGATTCT
GTGGATAACCGTATTACCGCCTTTGAGTGAGCTGATACCGCTCGC
CGCAGCCGAACGACCGAGCGCAGCGAGTCAGTGAGCGAGGAAGCG
GAAGAGCGCCTGATGCGGTATTTTCTCCTTACGCATCTGTGCGGT
ATTTCACACCGCATATGGTGCACTCTCAGTACAATCTGCTCTGAT
GCCGCATAGTTAAGCCAGTATACACTCCGCTATCGCTACGTGACT
GGGTCATGGCTGCGCCCCGACACCCGCCAACACCCGCTGACGCGC
CCTGACGGGCTTGTCTGCTCCCGGCATCCGCTTACAGACAAGCTG
TGACCGTCTCCGGGAGCTGCATGTGTCAGAGGTTTTCACCGTCAT
CACCGAAACGCGCGAGGCAGCTGCGGTAAAGCTCATCAGCGTGGT
CGTGAAGCGATTCACAGATGTCTGCCTGTTCATCCGCGTCCAGCT
CGTTGAGTTTCTCCAGAAGCGTTAATGTCTGGCTTCTGATAAAGC
GGGCCATGTTAAGGGCGGTTTTTTCCTGTTTGGTCACTGATGCCT
CCGTGTAAGGGGGATTTCTGTTCATGGGGGTAATGATACCGATGA
AACGAGAGAGGATGCTCACGATACGGGTTACTGATGATGAACAAG
AGGACATCCGGTAAGAGGACATCCGGTCAAATAAAACGAAAGGCT
CAGTCGAAAGACTGGGCCTTTCGTTTTATGGAAAGAAGAGAAACC
GGGACTGGTTCGTTTTGCTGAGGAGACTTAGGGACTCTCACAGCT
AACACCACGTCGTCCCTATCTGCTGCCCTAGGTCTATGAGTGGTT
GCTGGATAACTTTACGGGCATGCATAAGGCTCGTATAATATATTC
AGGGAGACCACAACGGTTTCCCTCTACAAATAATTTTGTTTAACT
TTTACTAGAGTGGGACCCTACCTGCAGGTGCAGTAAAGGAAAAAA
AAAATGAAAAAATTATTATTTGCGATACCGCTGTGCTTAAGCTAC
GAAACTGAAATCTTGACCGTCGAATATGGTCTGCTGCCAATCGGC
AAGATTGTTGAAAAACGTATTGAATGTACGGTCTACTCAGTGGAT
AACAACGGCAATATCTACACCCAGCCGGTGGCCCAGTGGCATGAC
CGTGGTGAACAGGAAGTGTTCGAATATTGTCTGGAAGACGGATCT
TTAATCCGTGCCACAAAGGATCACAAATTTATGACTGTAGATGGT
CAGATGCTCCCAATCGACGAAATTTTTGAACGCGAATTAGACCTG
ATGCGCGTGGATAATCTCCCGAATTCCGGAGGATCTAGCCCAACC
TCTGGCTCTGAGTAGGGCGGAACAGGATCTAGCGGGGGTTCAAGC
GAGAGTGCAACACCTGAAAGCAGCGGGACAAAAGCTGGCTCTGAG
TAGGGCGGAACAAGCGGTGGCTCAATCAAAATTGCCACGCGTAAA
TATTTAGGCAAACAGAATGTTTATGATATCGGTGTCGAGCGCGAT
CATAATTTCGCGCTGAAAAACGGATTTATAGCTAGCAATTGTTTC
AACGTTGTTCCTTTCTATTCTCACTCCGCCGAAACTGTTGAAAGT
TGTTTAGCAAAACCCCATACAGAAAATTCATTTACTAACGTCTGG
AAAGACGACAAAACTTTAGATCGTTACGCTAACTATGAGGGCTGT
CTGTGGAATGCTACAGGCGTTGTAGTTTGTACTGGTGACGAAACT
CAGTGTTACGGTACATGGGTTCCTATTGGGCTTGCTATCCCTGAA
AATGAGGGTGGTGGCTCTGAGGGTGGCGGTTCTGAGGGTGGCGGT
TCTGAGGGTGGCGGTACTAAACCTCCTGAGTACGGTGATACACCT
ATTCCGGGCTATACTTATATCAACCCTCTCGACGGCACTTATCCG
CCTGGTACTGAGCAAAACCCCGCTAATCCTAATCCTTCTCTTGAG
GAGTCTCAGCCTCTTAATACTTTCATGTTTCAGAATAATAGGTTC
CGAAATAGGCAGGGGGCATTAACTGTTTATACGGGCACTGTTACT
CAAGGCACTGACCCCGTTAAAACTTATTACCAGTACACTCCTGTA
TCATCAAAAGCCATGTATGACGCTTACTGGAACGGTAAATTCAGA
GACTGCGCTTTCCATTCTGGCTTTAATGAGGATCCATTCGTTTGT
GAATATCAAGGCCAATCGTCTGACCTGCCTCAACCTCCTGTCAAT
GCTGGCGGCGGCTCTGGTGGTGGTTCTGGTGGCGGCTCTGAGGGT
GGTGGCTCTGAGGGTGGCGGTTCTGAGGGTGGCGGCTCTGAGGGA
GGCGGTTCCGGTGGTGGCTCTGGTTCCGGTGATTTTGATTATGAA
AAGATGGCAAACGCTAATAAGGGGGCTATGACCGAAAATGCCGAT
GAAAACGCGCTACAGTCTGACGCTAAAGGCAAACTTGATTCTGTC
GCTACTGATTACGGTGCTGCTATCGATGGTTTCATTGGTGACGTT
TCCGGCCTTGCTAATGGTAATGGTGCTACTGGTGATTTTGCTGGC
TCTAATTCCCAAATGGCTCAAGTCGGTGACGGTGATAATTCACCT
TTAATGAATAATTTCCGTCAATATTTACCTTCCCTCCCTCAATCG
GTTGAATGTCGCCCTTTTGTCTTTGGCGCTGGTAAACCTTACGAG
TTCAGTATCGACTGCGATAAGATCAACCTGTTCCGCGGTGTCTTT
GCGTTTCTTTTATATGTTGCCACCTTTATGTATGTATTTTCTACG
TTTGCTAACATACTGCGTAATAAGGAGTCTTAATGAAATTTGGAA
ACTTTTTGCTTACATACCAACCTCCCCAATTTTCCCAAACAGAGG
TAATGAAACGTTTGGTTAAATTAGGTCGCATCTCTGAGGAGTGTG
GTTTTGATACCGTATGGTTACTGGAGCATCATTTCACGGAGTTTG
GTTTGCTTGGTAACCCTTATGTCGCTGCTGCATATTTACTTGGCG
CGACTAAAAAATTGAATGTAGGAACTGCCGCTATTGTTCTTCCCA
CAGCCCATCCAGTACGCCAACTTGAAGATGTGAATTTATTGGATC
AAATGTCAAAAGGACGATTTCGGTTTGGTATTTGCCGAGGGCTTT
ACAACAAGGACTTTCGCGTATTCGGCACAGATATGAATAACAGTC
GCGCCTTAGCGGAATGCTGGTACGGGCTGATAAAGAATGGCATGA
CAGAGGGATATATGGAAGCTGATAATGAACATATCAAGTTCCATA
AGGTAAAAGTAAACCCCGCGGCGTATAGCAGAGGTGGCGCACCGG
TTTATGTGGTGGCTGAATCAGCTTCGACGACTGAGTGGGCTGCTC
AATTTGGCCTACCGATGATATTAAGTTGGATTATAAATACTAACG
AAAAGAAAGCACAACTTGAGCTTTATAATGAAGTGGCTCAAGAAT
ATGGGCACGATATTCATAATATCGACCATTGCTTATCATATATAA
CATCTGTAGATCATGACTCAATTAAAGCGAAAGAGATTTGCCGGA
AATTTCTGGGGCATTGGTATGATTCTTATGTGAATGCTACGACTA
TTTTTGATGATTCAGACCAAACAAGAGGTTATGATTTCAATAAAG
GGCAGTGGCGTGACTTTGTATTAAAAGGACATAAAGATACTAATC
GCCGTATTGATTACAGTTACGAAATCAATCCCGTGGGAACGCCGC
AGGAATGTATTGACATAATTCAAAAAGACATTGATGCTACAGGAA
TATCAAATATTTGTTGTGGATTTGAAGCTAATGGAACAGTAGACG
AAATTATTGCTTCCATGAAGCTCTTCCAGTCTGATGTCATGCCAT
TTCTTAAAGAAAAACAACGTTCGCTATTATATTATGGCGGTGGCG
GTAGCGGCGGTGGCGGTAGCGGCGGTGGCGGTAGCGGCGGTGGCG
GTAGCAAATTTGGATTGTTCTTCCTTAACTTCATCAATTCAACAA
CTGTTCAAGAACAGAGTATAGTTCGCATGCAGGAAATAACGGAGT
ATGTTGATAAGTTGAATTTTGAACAGATTTTAGTGTATGAAAATC
ATTTTTCAGATAATGGTGTTGTCGGCGCTCCTCTGACTGTTTCTG
GTTTTCTGCTCGGTTTAACAGAGAAAATTAAAATTGGTTCATTAA
ATCACATCATTACAACTCATCATCCTGTCCGCATAGCGGAGGAAG
CTTGCTTATTGGATCAGTTAAGTGAAGGGAGATTTATTTTAGGGT
TTAGTGATTGCGAAAAAAAAGATGAAATGCATTTTTTTAATCGCC
CGGTTGAATATCAACAGCAACTATTTGAAGAGTGTTATGAAATCA
TTAACGATGCTTTAACAACAGGCTATTGTAATCCAGATAACGATT
TTTATAGCTTCCCTAAAATATCTGTAAATCCCCATGCTTATACGC
CAGGCGGACCTCGGAAATATGTAACAGCAACCAGTCATCATATTG
TTGAGTGGGCGGCCAAAAAAGGTATTCCTCTCATCTTTAAGTGGG
ATGATTCTAATGATGTTAGATATGAATATGCTGAAAGATATAAAG
CCGTTGCGGATAAATATGACGTTGACCTATCAGAGATAGACCATC
AGTTAATGATATTAGTTAACTATAACGAAGATAGTAATAAAGCTA
AACAAGAGACGCGTGCATTTATTAGTGATTATGTTCTTGAAATGC
ACCCTAATGAAAATTTCGAAAATAAACTTGAAGAAATAATTGCAG
AAAACGCTGTCGGAAATTATACGGAGTGTATAACTGCGGCTAAGT
TGGCAATTGAAAAGTGTGGTGCGAAAAGTGTATTGCTGTCCTTTG
AACCAATGAATGATTTGATGAGCCAAAAAAATGTAATCAATATTG
TTGATGATAATATTAAGAAGTACCACACGGAATATACCTAAACGG
AGCCAATGTACGCAAAAAACCCCGCTTCAGCGGGGTTTTTTCGCC
AAAAAAAACCCCGCCCTGTCAGGGGCGGGGTTTTTTTTTAGGGCC
CTACTGACTGTAAAAAAATAAACGATGCCCCTTAAAGCAGAAGCT
TTAAGGGGCAGAGCGTTGCGGCACATCTTTTCAGACGGCCTTATT
GTAGCAACGGTTCTCATTTCGTTTCCGAAATGAGAAAGGGAGCTA
CAACCTGGCTCTGAGTAGGGCGGAACACCACACAACATACGAGCC
GGAAGCATAAAGTGTAAAGCCTGGGGTGCCTAATGAGTGAGCTAA
CTCACATTAATTGCGTTGCGCTCACTGCCCGCTTTCCAGTCGGGA
AACCTGTCGTGCCAGCTGCATTAATGAATCGGCCAACGCGCGGGG
AGAGGCGGTTTGCGTATTGGGCGCACTTAATTAACGGCACTGCGA
AAAAACCCCGCCGAAGCGGGGTTTTTTGCGAAATATAATGACCCT
CTTGATAACCCAAGAGGGCATTTTTTAATGCCCATGGCGTTTACC
AATGCTTAATCAGTGAGGCACCTATCTCAGCGATCTGTCTATTTC
GTTCATCCATAGTTGCCTGACTCCCCGTCGTGTAGATAACTACGA
TACGGGAGGGCTTACCATCTGGCCCCAGTGCTGCAATGATACCGC
GAGACCCACGCTCACCGGCTCCAGATTTATCAGCAATAAACCAGC
CAGCCGGAAGGGCCGAGCGCAGAAGTGGTCCTGCAACTTTATCCG
CCTCCATCCAGTCTATTAATTGTTGCCGGGAAGCTAGAGTAAGTA
GTTCGCCAGTTAATAGTTTGCGCAACGTTGTTGCCATTGCTACAG
GCATCGTGGTGTCACGCTCGTCGTTTGGTATGGCTTCATTCAGCT
CCGGTTCCCAACGATCAAGGCGAGTTACATGATCCCCCATGTTGT
GCAAAAAAGCGGTTAGCTCCTTCGGTCCTCCGATCGTTGTCAGAA
GTAAGTTGGCCGCAGTGTTATCACTCATGGTTATGGCAGCACTGC
ATAATTCTCTTACTGTCATGCCATCCGTAAGATGCTTTTCTGTGA
CTGGTGAGTACTCAACCAAGTCATTCTGAGAATAGTGTATGCGGC
GACCGAGTTGCTCTTGCCCGGCGTCAATACGGGATAATACCGCGC
CACATAGCAGAACTTTAAAAGTGCTCATCATTGGAAAACGTTCTT
CGGGGCGAAAACTCTCAAGGATCTTACCGCTGTTGAGATCCAGTT
CGATGTAACCCACTCGTGCACCCAACTGATCTTCAGCATCTTTTA
CTTTCACCAGCGTTTCTGGGTGAGCAAAAACAGGAAGGCAAAATG
CCGCAAAAAAGGGAATAAGGGCGACACGGAAATGTTGAATACTCA
TACTCTTCCTTTTTCAATATTATTGAAGCATTTATCAGGGTTATT
GTCTCATGAGCGGATACATATTTGAATGTATTTAGAAAAATAAAC
AAATAGGGGTTCCGCGCACATTTCCCCGAAAAGTGCCACCTGACG
TCTAAGAAACCATTATTATCATGACATTAACCTATAAAAATAGGC
GTATCACGAGGCCCTTAGGCCAAATAGGCCGT

The sequences of these exemplary vectors are provided below, as SEQ ID NOs: 7-10. In some embodiments, vectors are provided that comprise a nucleic acid sequence that is at least 80%, 85%, 90%, 92.5%, 95%, 97%, 98%, or 99% identical to any one of SEQ ID NOs: 7-10. In some embodiments, any of these vectors comprise any of the sequences set forth as SEQ ID NOs: 7-10.

Vectors may be introduced and propagated in a prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-base editors. In some embodiments, the promoter is a pBAD promoter.

Fusion expression vectors also may be used to express the base editors of the disclosure. Such vectors generally add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the base editor. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.), and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods In Enzymology 185, Academic Press, San Diego, Calif. (1990) 60-89). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific: Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter. U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

In some embodiments, any of the disclosed vectors may comprise a minimal minute virus of mice (MVM) intron. In some embodiments, the MVM is positioned 5′ of the promoter and 3′ of the sequence encoding the sequence of interest, e.g., a sequence encoding any of the disclosed base editors.

In some embodiments, a vector of the present disclosure comprises a nucleic acid coding sequence that encodes a gIII protein comprising an in cis split intein pair connected by a polynucleotide insert sequence, at least 1 protospacer sequence, and at least 1 PAM sequence. In some embodiments, the in cis intein pair is inserted between nucleotide positions 30 and 31 of the coding sequence of gill protein. In other embodiments, the in cis intein pair is inserted between nucleotide positions 54 and 55 of the coding sequence of gill protein. In some embodiments, the in cis intein comprises Int-N and Int-C of an intein from N. punciforme (Npu). In some embodiments, the polynucleotide insert sequence is between 32-121 amino acids in length. In some embodiments, the polynucleotide insert sequence is 32 amino acids in length. In some embodiments, the polynucleotide insert sequence comprises at least 1 or at least 2 stop codons.

In some embodiments, the nucleic acid sequence has at least 80%, at least 85%, at least 90%, at least 95%, at least 98%, or at least 99% identity to the nucleic acid sequence of SEQ ID NO: 8. In some embodiments, the nucleic acid sequence comprises the nucleic acid sequence of SEQ ID NO: 8.

In some embodiments, the N-terminal region of the coding sequence comprises altered codon usage. In some embodiments, the N-terminal region comprises a sub-region of altered nucleotide homology. In some embodiments, the N-terminal region comprises a sub-region of altered nucleotide homology relative to gene IV (gVI) in the phage genome. In some embodiments, the nucleic acid sequence has at least 80%, at least 85%, at least 90%, at least 95%, at least 98%, or at least 99% identity to the nucleic acid sequence of SEQ ID NO: 9. In some embodiments, the nucleic acid sequence comprises the nucleic acid sequence of SEQ ID NO: 9. In some embodiments, there are 2 protospacers, each flanked by a PAM sequence and comprising alternate sequence identity at PAM nucleic acid positions 1-3 and 7.

In some embodiments, the nucleic acid sequence has at least 80%, at least 85%, at least 90%, at least 95%, at least 98%, or at least 99% identity to the nucleic acid sequence of SEQ ID NO: 7. In some embodiments, the nucleic acid sequence comprises the nucleic acid sequence of SEQ ID NO: 7.

In some aspects of the present disclosure, the nucleic acid sequence that encodes a fusion protein comprising a TadA8e domain and a dNme2Cas9 domain connected by a polynucleotide insert sequence and an in trans intein. In some embodiments, the in trans intein is gp41-8.

In some embodiments, the nucleic acid sequence has at least 80%, at least 85%, at least 90%, at least 95%, at least 98%, or at least 99% identity to the nucleic acid sequence of SEQ ID NO: 10. In some embodiments, the nucleic acid sequence comprises the nucleic acid sequence of SEQ ID NO: 10.

### Methods of Treatment

The present disclosure provides methods for the treatment of a subject diagnosed with a disease associated with or caused by a point mutation that can be corrected by a fusion protein provided herein (e.g., a base editor fusion protein comprising any of the Nme2Cas9 variants described herein, and a deaminase). For example, in some embodiments, a method is provided that comprises administering to a subject having such a disease, e.g., a disease such as cancer associated with a point mutation, an effective amount of a base editor, and a gRNA that forms a complex with the base editor, that corrects the point mutation or introduces a deactivating mutation into a disease-associated gene. In some embodiments, a method is provided that comprises administering to a subject having such a disease, e.g., a cancer associated with a point mutation, an effective amount of a base editor-gRNA complex that corrects the point mutation or introduces a deactivating mutation into a disease-associated gene. Further provided herein are methods comprising administering to a subject one or more vectors that contains a nucleotide sequence that expresses the base editor and gRNA that forms a complex with the base editor.

In some embodiments, the disease is a proliferative disease. In some embodiments, the disease is a genetic disease. In some embodiments, the disease is a neoplastic disease. In some embodiments, the disease is a metabolic disease. Other diseases that can be treated by correcting a point mutation or introducing a deactivating mutation into a disease-associated gene will be known to those of skill in the art, and the disclosure is not limited in this respect.

The present disclosure provides methods for the treatment of additional diseases or disorders, e.g., diseases or disorders that are associated or caused by a point mutation that can be corrected by base editing. Some such diseases are described herein, and additional suitable diseases that can be treated with the strategies and fusion proteins (e.g., base editors) provided herein will be apparent to those of skill in the art based on the present disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation, sickle cell disease, progeria, cystic fibrosis, and ornithine transcarbamylase (OTC) deficiency. In some embodiments, the disclosed compositions and methods may be suitable for editing a clinically relevant point mutation in sickle cell disease, such as HBBs, the Makassar allele.

Exemplary methods for the treatment of diseases, disorders or conditions using one or more cytidine or adenine base editors by correcting a point mutation or introducing a deactivating mutation into a disease-associated gene are disclosed in International Publication Nos. WO 2021/222318, published Nov. 4, 2021: WO 2021/183693, published Sep. 16, 2021; WO 2021/158999, published Aug. 12, 2021; WO 2020/051360, published Mar. 12, 2020; and WO 2019/079347, published Apr. 25, 2019, each of which is herein incorporated by reference.

In some aspects, the present disclosure provides uses of any one of the fusion proteins (e.g., base editors) described herein, and a guide RNA targeting this base editor to a target C:G base pair in a nucleic acid molecule, in the manufacture of a kit for base editing, wherein the base editing comprises contacting the nucleic acid molecule with the base editor and guide RNA under conditions suitable for the substitution of the cytosine (C) of the C:G nucleobase pair with a thymine (T). In some embodiments of these uses, the nucleic acid molecule is a double-stranded DNA molecule. In some embodiments, the step of contacting induces separation of the double-stranded DNA at a target region. In some embodiments, the step of contacting further comprises nicking one strand of the double-stranded DNA, wherein the one strand comprises an unmutated strand that comprises the G of the target C:G nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the fusion proteins described herein as a medicament. The present disclosure also provides uses of any one of the complexes of fusion proteins and guide RNAs described herein as a medicament.

### Pharmaceutical Compositions

Other aspects of the present disclosure relate to pharmaceutical compositions comprising any of the fusion proteins, guide RNAs, complexes, systems, polynucleotides, vectors, and/or cells described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used herein, the term “pharmaceutically-acceptable carrier” (or “pharmaceutically acceptable excipient”) means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate: (4) powdered tragacanth; (5) malt; (6) gelatin; (7) lubricating agents, such as magnesium stearate, sodium lauryl sulfate and talc: (8) excipients, such as cocoa butter and suppository waxes; (9) oils, such as peanut oil, cottonseed oil, safflower oil, sesame oil, olive oil, corn oil and soybean oil: (10) glycols, such as propylene glycol: (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide, (15) alginic acid: (16) pyrogen-free water: (17) isotonic saline; (18) Ringer's solution: (19) ethyl alcohol; (20) pH buffered solutions; (21) polyesters, polycarbonates and/or polyanhydrides: (22) bulking agents, such as polypeptides and amino acids; (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. Terms such as “excipient”, “carrier”, “pharmaceutically acceptable carrier,” “pharmaceutically acceptable excipient,” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533: Seflon, 1989, CRC Crit. Ref Biomed Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984), Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurmsurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther: 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

The disclosure also provides methods for delivering a base editor described herein (e.g., in the form of an evolved base editor as described herein, or a vector or construct encoding same) into a cell. Such methods may involve transducing (e.g., via transfection) cells with a plurality of complexes each comprising a base editor and a gRNA molecule. In some embodiments, the gRNA is bound to the napDNAbp domain (e.g., nCas9 domain) of the base editor. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence. In certain embodiments, the methods involve the transfection of nucleic acid constructs (e.g., plasmids and mRNA constructs) that each (or together) encode the components of a complex of base editor and gRNA molecule. In certain embodiments, any of the disclosed base editors and a gRNA are administered as a protein:RNA complex, such as a ribonucleoprotein complex. In some embodiments, any of the disclosed base editors are administered as an mRNA construct, along with the gRNA molecule. In particular embodiments, administration to cells is achieved by electroporation or lipofection.

In certain embodiments of the disclosed methods, a nucleic acid construct (e.g., an mRNA construct) that encodes the base editor is transfected into the cell separately from the construct that encodes the gRNA molecule. In certain embodiments, these components are encoded on a single construct and transfected together. In other embodiments, the methods disclosed herein involve the introduction into cells of a complex comprising a base editor and gRNA molecule that has been expressed and cloned outside of these cells.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

In another aspect, the disclosure discloses a pharmaceutical composition comprising any one of the presently disclosed vectors. In certain embodiments, the pharmaceutical composition further comprises a pharmaceutically acceptable excipient. In certain embodiments, the pharmaceutical composition further comprises a lipid and/or polymer. In certain embodiments, the lipid and/or polymer is cationic. The preparation of such lipid particles is well known. See, e.g., U.S. Pat. Nos. 4,880,635; 4,906,477; 4,911,928; 4,917,951; 4,920,016; 4,921,757; and 9,737,604, each of which is incorporated herein by reference. In some embodiments, the pharmaceutical composition comprises a Cas protein, a fusion protein, a guide RNA, a complex, a polynucleotide, a vector, an rAAV particle, or a cell, and a pharmaceutically acceptable excipient. In some embodiments, the pharmaceutical composition comprises a Cas protein and a pharmaceutically acceptable excipient. In some embodiments, the pharmaceutical composition comprises a Cas protein, a fusion protein, and a pharmaceutically acceptable excipient. In some embodiments, the pharmaceutical composition comprises a Cas protein, a fusion protein, a guide RNA, and a pharmaceutically acceptable excipient. In some embodiments, the pharmaceutical composition comprises a Cas protein, a fusion protein, a guide RNA, a complex, and a pharmaceutically acceptable excipient. In some embodiments, the pharmaceutical composition comprises a Cas protein, a fusion protein, a guide RNA, a complex, a polynucleotide, and a pharmaceutically acceptable excipient. In some embodiments, the pharmaceutical composition comprises a Cas protein, a fusion protein, a guide RNA, a complex, a polynucleotide, a vector, and a pharmaceutically acceptable excipient. In some embodiments, the pharmaceutical composition comprises a Cas protein, a fusion protein, a guide RNA, a complex, a polynucleotide, a vector, an rAAV particle, and a pharmaceutically acceptable excipient. In some embodiments, the pharmaceutical composition comprises a Cas protein, a fusion protein, a guide RNA, a complex, a polynucleotide, a vector, an rAAV particle, or a cell, and a pharmaceutically acceptable excipient. In some embodiments, the pharmaceutical composition may be used in medicine. In some embodiments, the pharmaceutical composition may be used in the manufacture of a medicament for the treatment of a disease or disorder. In some embodiments, the disease or disorder is sickle cell disease (SCD). In some embodiments, the sickle cell disease (SCD) is caused by a mutation in a gene locus. In some embodiments, the gene locus is a mutation of the mammalian β-globin (HBB) gene locus at amino acid position 6, relative to the wild-type mammalian β-globin (HBB) gene. In some embodiments, the mutation of the mammalian β-globin (HBB) gene locus at amino acid position 6, is a glutamate to valine mutation.

Exemplary methods of delivery of nucleic acids include lipofection, nucleofection, electoporation (e.g., MaxCyte electroporation), stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g. in vitro or ex vivo administration) or target tissues (e.g. in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther: 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of base editors markedly increases the DNA specificity of base editing. RNP delivery of base editors leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368: WO 93/24641; Kotin, Human Gene Therapy 5:793-801 (1994): Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003/0087817, published May 8, 2003, International Patent Application No. WO 2016/205764, published Dec. 22, 2016, International Patent Application No. WO 2018/071868, published Apr. 19, 2018, U.S. Patent Publication No. 2018/0127780, published May 10, 2018, and International Publication No. WO2020/236982, published Nov. 26, 2020, the disclosures of each of which are incorporated herein by reference.

In some aspects of the present disclosure, relate to a polynucleotide or polynucleotides encoding a Cas protein, a fusion protein, a guide RNA, or a complex. In some embodiments, the polynucleotide comprises a (i) first segment encoding the fusion protein and (ii) a second segment encoding the guide RNA. In some embodiment, the first segment encodes the guide RNA and the second segment encodes the fusion protein. In some embodiments, the polynucleotide is in a vector. In some embodiments, the vector is an adeno-associated viral (AAV) vector. In some embodiments, the orientation of the second segment is reversed relative to the first segment. In some embodiments, the orientation of the first segment is reversed relative to the second segment.

Recombinant Adeno-Associated Viral (rAAV) Vectors

Aspects of the presently disclosed delivery methods relate to using recombinant adeno-associated virus vectors for the delivery of any of the disclosed nucleic acid molecules. The rAAV particles of the present disclosure comprise a rAAV vector (i.e., a recombinant genome of the rAAV) encapsidated in the viral capsid proteins. See U.S. Patent Publication No. 2018/0127780, published May 10, 2018, and PCT Publication No. WO 2020/236982, published Nov. 26, 2020, the disclosures of each of which are incorporated herein by reference.

In some embodiments, the AAV nucleic acid vector is single-stranded. In some embodiments, the AAV nucleic acid vector is self-complementary. In various embodiments, the rAAV vectors of the disclosure do not contain any inteins.

In some embodiments, viral sequences that facilitate integration comprise Inverted Terminal Repeat (ITR) sequences. In some embodiments, nucleic acid molecule is flanked on each side by an ITR sequence. In some embodiments, the nucleic acid vector further comprises a region encoding an AAV Rep protein as described herein, either contained within the region flanked by ITRs or outside the region. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV8 or AAV9. In some embodiments, in methods of packaging any of the disclosed rAAV particles, a nucleic acid plasmid, such as a helper plasmid, that comprises a region encoding a Rep protein and/or a Cap (capsid) protein is provided.

In various embodiments, any of the disclosed base editor (or fusion protein) constructs may be engineered for delivery in one or more AAV vectors. Any of the disclosed AAV vectors may comprise 5′ and 3′ inverted terminal repeats (ITRs) that flank the polynucleotide (or construct) encoding any of the disclosed base editors. In some embodiments, any of the base editor constructs may be engineered for delivery in a single rAAV vector. In some embodiments, any of the disclosed base editor constructs has a length of 4.9 kilobases or less, and as such may be packaged into a single AAV vector, while being flanked by ITRs. In some embodiments, any of the disclosed base editor constructs has a length of between about 4.65 kb, about 4.70 kb, about 4.725 kb, about 4.75 kb, about 4.80 kb, about 4.825 kb, about 4.85 kb, or about 4.90 kb between the 5′ and 3′ ITRs. In some embodiments, any of the disclosed base editor constructs has a length of between 4.7 kb and 4.9 kb, such as about 4.8 kb.

In some embodiments, any of the disclosed base editor constructs or rAAV vectors containing a polynucleotide encoding a base editor comprises a first segment encoding the base editor, and further comprises a second nucleic acid segment encoding a guide RNA, such as a single-guide RNA. In some embodiments, the orientation of this gRNA-encoding (second) nucleic acid segment is reversed relative to the orientation of the segment encoding the base editor. In some embodiments, the first nucleic acid segment is operably controlled by a first promoter, and the second nucleic acid segment is operably controlled by a second promoter (e.g., a U6 promoter). In several embodiments, the first promoter is different from the second promoter. The disclosure provides single AAV vectors comprising any of the above-contemplated base editor constructs.

The disclosure provides recombinant AAV particles comprising any of the disclosed AAV vectors. These rAAV particles may comprise an AAV vector and a capsid protein. The capsid protein may be of any serotype.

Accordingly, an rAAV particle as related to any of the disclosed uses, methods, and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor that is carried by the rAAV into a cell) that is to be delivered to a cell. An rAAV may be chimeric.

As used herein, the serotype of an rAAV particle refers to the serotype of the capsid protein of the recombinant virus. In some embodiments, the rAAV particles disclosed herein comprise an rAAV2, rAAV3, rAAV3B, rAAV4, rAAV5, rAAV6, rAAV8, rAAV9, rAAV10, rPHP.B, rPHP.eB, or rAAV9 particle, or a variant thereof. In particular embodiments, the disclosed rAAV particles are rAAV8 or rAAV9 particles.

Non-limiting examples of serotype derivatives and pseudotypes include rAAV2/1, rAAV2/5, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol. Ther: 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

ITR sequences and plasmids containing ITR sequences are known in the art and commercially available (see, e.g., products and services available from Vector Biolabs, Philadelphia, PA: Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Nal Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference.

In some embodiments, the rAAV vector of the present disclosure comprises one or more regulatory elements to control the expression of the heterologous nucleic acid region (e.g., promoters, transcriptional terminators, and/or other regulatory elements). In some embodiments, the first and/or second nucleotide sequence is operably linked to one or more (e.g., 1, 2, 3, 4, 5, or more) transcriptional terminators. Non-limiting examples of transcriptional terminators that may be used in accordance with the present disclosure include transcription terminators (or polyadenylation signals) of the bovine growth hormone gene (bGH), human growth hormone gene (hGH), SV40, CW3, ϕ, or combinations thereof. In exemplary embodiments, the transcriptional terminator is an SV40 polyadenylation signal. In exemplary embodiments, the transcriptional terminator does not contain a posttranscription response element, such as WPRE element.

In some aspects, provided herein are methods of making (or manufacturing, or packaging) any of the disclosed rAAV particles. rAAV particles may be manufactured according to any method known in the art. Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US 2007-0015238 and US 2012-0322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

In some embodiments, the base editors may be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their trans-splicing inside the cell and the concomitant restoration of the complete, functioning ABE. These split intein-based methods may overcome several barriers to in vivo delivery. For example, the DNA encoding some base editors is larger than the recombinant AAV (rAAV) packaging limit, and so requires different solutions. One such solution is formulating the editor fused to split intein pairs that are packaged into two separate rAAV particles that, when co-delivered to a cell, reconstitute the functional editor protein.

In some embodiments, the base editor may be divided into two halves at a split site. These two halves may be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their trans-splicing inside the cell and the concomitant restoration of the complete, functioning ABE.

Accordingly, in various embodiments, the base editors may be engineered as two half proteins (i.e., an ABE N-terminal half and a ABE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell. In some embodiments, the split intein may be a Nostoc punctiforme (Npu) trans-splicing DnaE intein, i.e., an Npu split intein. Accordingly, in some embodiments, the N-terminal and C-terminal portions of the split intein are NpuC and NpuN, respectively.

Other solutions entail encoding the editor, and further encoding a guide RNA, in a single AAV vector for packaging in a single rA AV particle. Accordingly, in some embodiments, any of the disclosed base editors may be encoded in a single AAV vector, without the use of any split points or inteins. Several other special considerations to account for the unique features of base editing are described, including the optimization of second-site nicking targets and properly packaging base editors into virus vectors, including lentiviruses and rAAV.

Accordingly, the disclosure provides rAAV vectors and rAAV vector particles that comprise expression constructs that encode any of the disclosed base editors. In exemplary embodiments, any of the disclosed base editors are delivered to one or more cells in a single rAAV particle.

In some aspects, the disclosure provides compositions containing a plurality of any of the disclosed rAAV particles. In some aspects, the disclosure provides host cells containing a plurality of any of the disclosed rAAV particles. In some embodiments, the host cells are mammalian cells, such as human cells. In other embodiments, the host cells are yeast cells, plant cells, or bacterial cells.

Methods of delivery to a target cell or target tissue of any of the disclosed rAAV particles and compositions and host cells comprising rAAV particles are known in the art. In some embodiments, any of the disclosed rAAV particles, host cells, or compositions are delivered to a subject, such as a mammalian subject. In some embodiments, the rAAV particles are delivered to a human subject.

In some embodiments, the disclosed rAAV particles and compositions are administered to a subject in a single injection, such as a single systemic injection. In some embodiments, the disclosed rAAV particles and compositions are administered to a subject in multiple injections. rAAV particles are known to transduce target tissues within days, but are typically allowed three to four weeks to complete transduction, genome integration, and clearance, from the cell. Accordingly, in some aspects, any of the disclosed rAAV particles or compositions are administered to a subject for a period of three weeks. in some aspects, any of the disclosed rAAV particles or compositions are administered to a subject for a period of between three and four weeks.

In some embodiments, any of the disclosed rAAV particles or compositions is administered to a subject or a target tissue in a therapeutically effective amount of about 10¹⁵, about 10¹⁴, about 10¹³, about 10¹², about 10¹¹, or less than about 10¹¹ vector genomes (vg) per kg weight of the subject. In some embodiments, the rAAV particles are administered in an amount of between 10¹⁵ and 10¹⁴, between 10¹⁴ and 101, between 101 and 101, between 10¹² and 10¹, or between 10¹² and 10¹¹ vgs per kg. In some embodiments, the rAAV particles are administered in an amount of between 10¹⁴ and 10¹¹ vgs per kg. In some embodiments, any of the disclosed rAAV particles or compositions is administered to a target tissue of a subject in a lower dose than is convention for dual AAV particle delivery, such as that described in PCT Publication No. WO 2020/236982, published Nov. 26, 2020 and Levy, J. M., et al. Nat Biomed Eng 4, 97-110 (2020).

In some embodiments, the disclosed rAAV particles provide for transduction of the target tissue to achieve expression and translation of the payload or transgene, e.g., a base editor in accordance with the present disclosure, for a sufficient duration to install desired mutations in the genome of a target cell. In some embodiments, the desired mutation is an A to G mutation. In some embodiments, the desired mutation is a C to T mutation. In some embodiments, the disclosed rAAV particles provide for sufficient expression and translation of the base editor transgene for a sufficient duration to install desired (on-target) mutations in the genome with a tolerable degree of off-target effects, such as bystander edits. In some embodiments, the disclosed rAAV particles provide for sufficient expression and translation of the base editor transgene for a sufficient duration to install desired mutations in the genome without appreciable off-target editing. In some embodiments, the disclosed rAAV particles provide for sufficient expression and translation of the base editor transgene for a sufficient duration to install desired mutations in the genome without appreciable bystander editing.

Suitable routes of administrating the disclosed compositions of rAAV particles include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, systemic, intravascular, intraosseus, periocular, intratumoral, intracerebral, parenteral, and intracerebroventricular administration. In some embodiments, the route of administration is systemic (intravenous). In some embodiments, the pharmaceutical composition described herein is administered locally to a diseased site.

Additional methods for the delivery of nucleic acids to cells are known to those skilled in the art. See, for example, US Pub. No. 2003/0087817, incorporated herein by reference.

It should be appreciated that any base editor, e.g., any of the base editors provided herein, may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a virus encoding a base editor), or transfected (e.g., with a plasmid encoding a base editor) with a nucleic acid that encodes a base editor, or the translated base editor. Such transduction may be a stable or transient transduction. In some embodiments, cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into cells through electroporation, transient (e.g., lipofection) and stable genome integration (e.g., piggybac) and viral transduction or other methods known to those of skill in the art.

### Kits and Cells

Some aspects of this disclosure provide kits comprising a nucleic acid construct comprising a nucleotide sequence encoding an adenosine deaminase capable of deaminating an adenosine in a deoxyribonucleic acid (DNA) molecule. In some embodiments, the nucleotide sequence encodes any of the adenosine deaminases provided herein. In some embodiments, the nucleotide sequence comprises a heterologous promoter that drives expression of the adenosine deaminase. The nucleotide sequence may further comprise a heterologous promoter that drives expression of the gRNA, or a heterologous promoter that drives expression of the base editor and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising a nucleic acid construct, comprising (a) a nucleotide sequence encoding a napDNAbp (e.g., a Cas9 domain) fused to an adenosine deaminase; or a base editor comprising a napDNAbp (e.g., Cas9 domain) and an adenosine deaminase as provided herein; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

In some embodiments, the kit comprises: (a) a nucleic acid sequence encoding the fusion protein: (b) a nucleic acid sequence encoding a gRNA; and (c) one or more heterologous promoters that drive the expression of the sequence of (a) and/or the sequence of (b).

Some embodiments of this disclosure provide cells comprising any of the compositions, base editors or complexes provided herein. In some embodiments, the cells comprise nucleotide constructs that encodes any of the base editors provided herein. In some embodiments, the cells comprise any of the nucleotides or vectors provided herein. In some embodiments, the cell is a stem cell. In some embodiments, the cell is a human stem cell, such as a human stem and progenitor cell (HSPC). In some embodiments, the cell is a mobilized (e.g., plerixafor-mobilized) peripheral blood HSPC.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. In some embodiments, the cell has been removed from a subject and contacted ex vivo with any of the disclosed base editors, complexes, vectors, or polynucleotides.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bc1-1, BC-3, IC21, DLD2, Raw264.7, NRK, NRK-52E, MRC5, MEF, Hep G2, HeLa B, HeLa T4, COS. COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-KI, CHO-K2, CHO-T, CHO Dhfr−/−. COR-L23, COR-L23/CPR, COR-L23/5010, COR-L23/R23, COS-7, COV-434, CMLT1, CMT, CT26, D17, DH82, DU145. DuCaP, EL4, EM2, EM3, EMT6/AR1, EMT6/AR10.0, FM3, H1299, H69, HB54, HB55, HCA2, HEK-293, HeLa, Hepalc 1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6. MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some aspects, the present disclosure provides uses of any one of the base editors described herein and a guide RNA targeting this base editor to a target A:T base pair in a nucleic acid molecule in the manufacture of a kit for nucleic acid editing, wherein the nucleic acid editing comprises contacting the nucleic acid molecule with the base editor and guide RNA under conditions suitable for the substitution of the adenine (A) of the A:T nucleobase pair with an guanine (G). In some embodiments of these uses, the nucleic acid molecule is a double-stranded DNA molecule. In some embodiments, the step of contacting of induces separation of the double-stranded DNA at a target region. In some embodiments, the step of contacting thereby comprises nicking one strand of the double-stranded DNA, wherein the one strand comprises an unmutated strand that comprises the T of the target A:T nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the base editors described herein as a medicament. The present disclosure also provides uses of any one of the complexes of base editors and guide RNAs described herein as a medicament.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### EXAMPLES

### Example 1

### Introduction

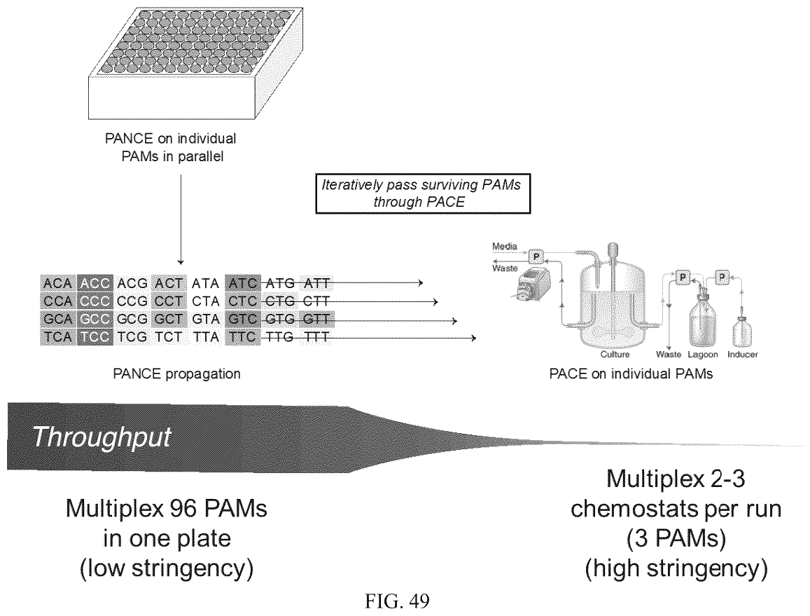

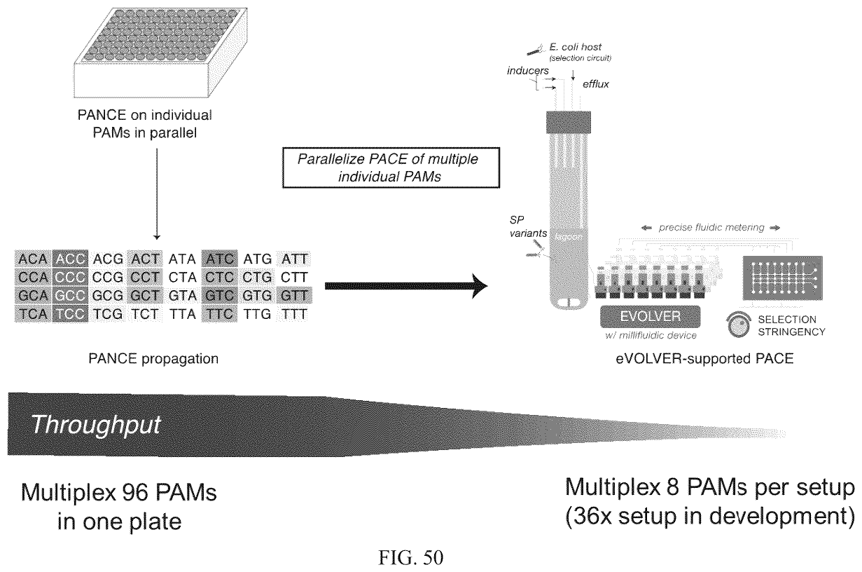

The directed evolution of Nme2Cas9²⁰ is reported, expanding its PAM scope from the N₄CC requirement of the wild-type protein to include most N₄YN sequences, where Y=C or T. To enable the evolution of this non-SpCas9 ortholog, three technologies were developed and integrated. First, a new, generalizable selection strategy requiring both PAM recognition and functional editing activity was established. Selections were then carried out in parallel across single PAM sequences using phage-assisted non-continuous evolution (PANCE)³ (FIGS. 49-50) and a novel, high-throughput eVOLVER-enabled²² phage-assisted continuous evolution (ePACE) platform. Lastly, a high-throughput base editing-dependent PAM profiling assay (BE-PPA) was developed to rapidly and thoroughly characterize evolving Nme2Cas9 variants and to guide evolutionary trajectories. With these developments, four Nme2Cas9 variants evolved that enabled robust precision genome editing at PAMs with a single specified pyrimidine nucleotide: eNme2-C(SEQ ID NO: 1), eNme2-C.NR (SEQ ID NO: 4), eNme2-T.1 (SEQ ID NO: 2), and eNme2-T.2 (SEQ ID NO: 3). The evolved Nme2 variants exhibited comparable (eNme2-T.1 (SEQ ID NO: 2) and eNme2-T.2 (SEQ ID NO: 3)) or more robust (eNme2-C(SEQ ID NO: 1)) base editing and lower off-target editing than SpRY, the only other engineered variant capable of accessing similar PAMs for a subset of target sites¹⁴. Together, these new variants offered broad PAM accessibility that was complementary to the suite of PAMs previously targetable by SpCas9-derived variants. Moreover, the selection strategy developed in this disclosure is highly scalable and general. Because of the lack of target site requirements, this selection could in principle be applied to evolve functional activities in any Cas ortholog or to optimize editing at a specific PAM or target site.

### Results

In some embodiments the continuous evolution system, PACE²³, in which the propagation of M13 bacteriophage was coupled to the desired activity of a protein of interest (POI), was used to evolve Nme2Cas9 variants with expanded pyrimidine-rich PAM scope. Previously, the PAM scope of SpCas9 variants was broadened using a one-hybrid, DNA-binding PACE circuit^{10,11}. In those efforts, SpCas9 variants encoded on selection phage (SP) capable of simply binding the target PAM(s) successfully produced gene III (gIII), a gene essential for phage propagation. The resulting SpCas9 variants could access most NR PAM sequences (where R=A or G), but efforts to apply the DNA-binding selection to evolve pyrimidine PAM recognition were less successful^{10,11}.

While this binding selection could be adapted to evolve Nme2Cas9, fundamental differences between the activities of SpCas9 and Nme2Cas9 could impede efforts to evolve the PAM scope of the latter. Nme2Cas9, and more broadly Type II-C Cas variants, may have slower nuclease kinetics relative to SpCas9¹⁶. This weaker nuclease activity is attributed to slower Cas9 helicase activity, as artificially introduced bulges mimicking partially unwound DNA in the PAM proximal region increase the cleavage rate of Type II-C Cas variants but not of SpCas9¹⁶. This theory is supported by observations that miniaturized SpCas9 variants with partially deleted domains have reduced DNA binding affinity that can also be rescued by the introduction of PAM-proximal bulges in target DNA²⁴. Because a primary motivation for broadening PAM compatibility is to improve the applicability of precision gene editing technologies that require DNA unwinding⁸, it is critical that a selection preserves or improves R-loop formation, maintenance, and nuclease activation. Notably, these Cas properties are dependent on domains outside of the PAM-interacting domain (PID), which has been the focus of rational engineering approaches^{12,14,17,18}. Together, this analysis suggests that while DNA-binding selections or PID engineering can yield robust SpCas9 variants with altered PAM compatibilities, the same type of binding-only selection applied to the evolution of Nme2Cas9 or similar Cas orthologs may not yield both desired PAM recognition and efficient downstream activity (FIG. 1A). Therefore, a new, functional selection in PACE for evolving PAM compatibility was needed.

### Development of a General Functional Selection for Evolving PAM Compatibility in PACE

To develop a functional selection for Cas9-based genome editing agents with altered PAM compatibilities, elements of a DNA-binding selection^{10,11} were integrated with base editing (BE) selection^{25,26}, such that both novel PAM recognition and subsequent BE within the protospacer are required to pass the selection. Although BE selections to evolve high-activity adenine and cytidine deaminases^{25,26} were previously developed, these selections placed targeted nucleotides within the coding sequence of T7 RNA polymerase (T7 RNAP). This selection strategy is not broadly applicable to evolve altered PAM compatibility since changing the target PAM and protospacer likely requires changing the coding sequence of T7 RNAP. Furthermore, evolved variants with high activity that edit over large activity windows may inadvertently alter the activity of T7 RNAP through bystander editing.

To address these limitations, a new selection strategy was designed in which the target protospacer and PAM could be fully specified without impacting the coding sequence of the gene responsible for selection survival (FIG. 1B). To achieve this level programmability, the splicing capabilities of inteins, protein elements that insert and remove themselves from other proteins in cis, were used which left only a small (˜3- to 10-aa) extein scar^{27,28}. In some embodiments, it was described that trans split-inteins could function effectively as cis splicing elements when the N- and C-inteins were fused together with a linker containing a programmed PAM and protospacer. The split-intein pair from N. punciforme (Npu)²⁹ was used, since it showed that gIII split after residue 10 (Leucine) with the Npu intein supports robust phage propagation after trans splicing³⁰.

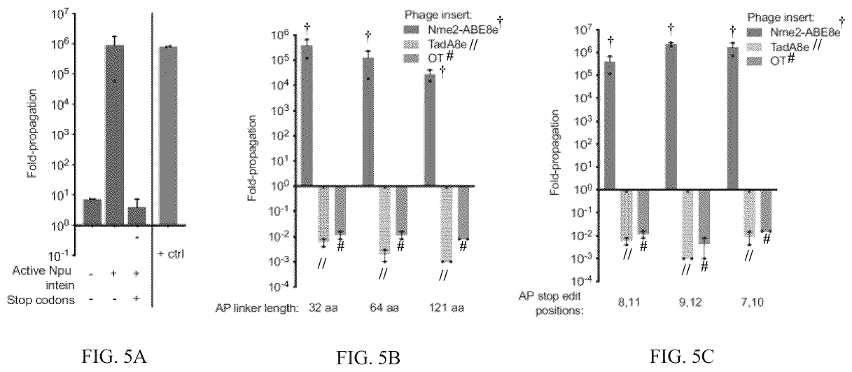

In some embodiments, it was determined that the reconfigured cis-splicing Npu intein supported phage propagation. An accessory plasmid (AP) was constructed with the N- and C-terminal halves of the Npu intein fused together with a flexible 32 amino acid (aa) linker and inserted into the coding sequence of gill after Leucine 10 under the control of the phage shock promoter (psp)³¹ (FIG. 1B). When infected with DgIII-phage, host cells containing the AP supported robust phage propagation in a splicing-dependent manner similar to cells containing psp-driven wild-type gill. Importantly, installation of stop codons within the linker sequence reduced phage propagation by >10⁵-fold relative to the unmutated construct (FIG. 5A), indicating that this selection, termed sequence-agnostic Cas PACE (SAC-PACE), should enable robust selection of variants capable of correcting targeted stop codons.

Next, it was assessed whether adenine base editing could support phage propagation in SAC-PACE. Indeed, on host cells harboring an AP containing gIII with two stop codons flanked by a cognate Nme2Cas9 N₄CC PAM, phage encoding dead Nme2Cas9 fused to the adenosine deaminase TadA8e²⁵ (Nme2-ABE8e) enriched 10²- to 10⁶-fold after overnight propagation, depending on the expression level of the gill-construct (FIG. 1C). In contrast, phage containing only TadA8e or a non-targeting gene de-enriched in these host cells below the limit of detection at any tested expression level, indicating a large base-editing dependent dynamic range for this selection.

To test the generality of the selection circuit, a series of APs containing linkers between 32 and 121 as or with stop codons placed at different positions within the protospacer were generated (FIGS. 5B and 5C). Although propagation decreased with increasing linker length, the maximum tested linker length of 121 aa still supported strong overnight propagation sufficient to support phage survival during PACE (>10⁴-fold)³. This linker length can encode up to 10 simultaneous protospacer/PAM combinations (23 to 30 nt in length) with at least 7 nt between targets, a spacing shown to be compatible for multiple Cas protein binding events¹². Together, these results suggested that the SAC-PACE selection is a highly flexible system that could be used to evolve the PAM scope of Cas variants.

A High-Throughput Platform for Phage-Assisted Continuous Evolution (ePACE)

Previous efforts to evolve SpCas9 on specific PAM sequences (NAG, NAC, NAT, etc.) yielded variants with both higher activity and specificity compared to variants evolved on a broad set of pooled PAMs¹¹. Evolving on specific PAM sequences using traditional PACE methodology, however, is limited by throughput, since PACE is inherently challenging to parallelize due to cost, space, and design complexity, requiring temperature-controlled rooms and fluid-handling equipment³³. This constraint limits the number of conditions that can be explored in a PACE campaign, a drawback given the difficulty of predicting the set of conditions that will evolve molecules with desired properties.

To address this throughput challenge and enable large-scale parallel PACE of Nme2Cas9 towards specific PAMs, ePACE was developed (FIGS. 1D and 14A-16D). The ePACE system combined the continuous mutagenesis and selection of PACE with the highly scalable, customizable, and automated eVOLVER continuous culture platform, which has already proven effective for directed evolution³⁴. Three key design features of eVOLVER made it an ideal choice for facilitating parallel PACE selections. First, eVOLVER enabled individual programmatic control of continuous culture conditions, allowing the platform to simultaneously operate PACE chemostat cell reservoirs and lagoons on a standard lab benchtop. Second, eVOLVER could scale in a cost-effective manner to arbitrary throughput, enabling large-scale parallelization of miniature PACE reactors. Lastly, the do-it-yourself and open-source nature of eVOLVER allow it to be rapidly adapted and reconfigured for novel actuation elements, making it amenable to the customization necessary to run PACE (FIGS. 14A-16D). Integrating PACE and eVOLVER enabled the simultaneous execution of PACE experiments across eight different PAMs in parallel. Given that PACE experiments typically required 1-2 weeks each, this 8-fold increase in throughput represents a 2- to 4-month reduction in experimental time compared to traditional single-lagoon PACE at a 10-fold reduction in cost.

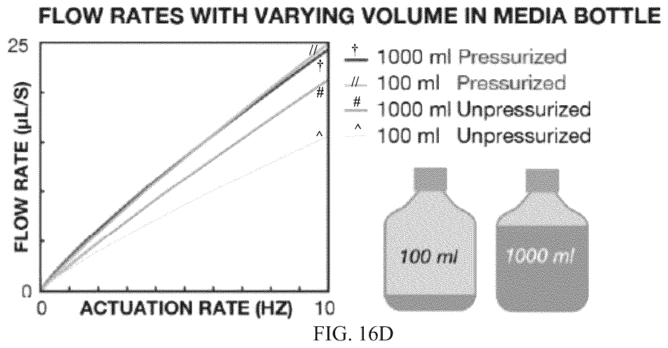

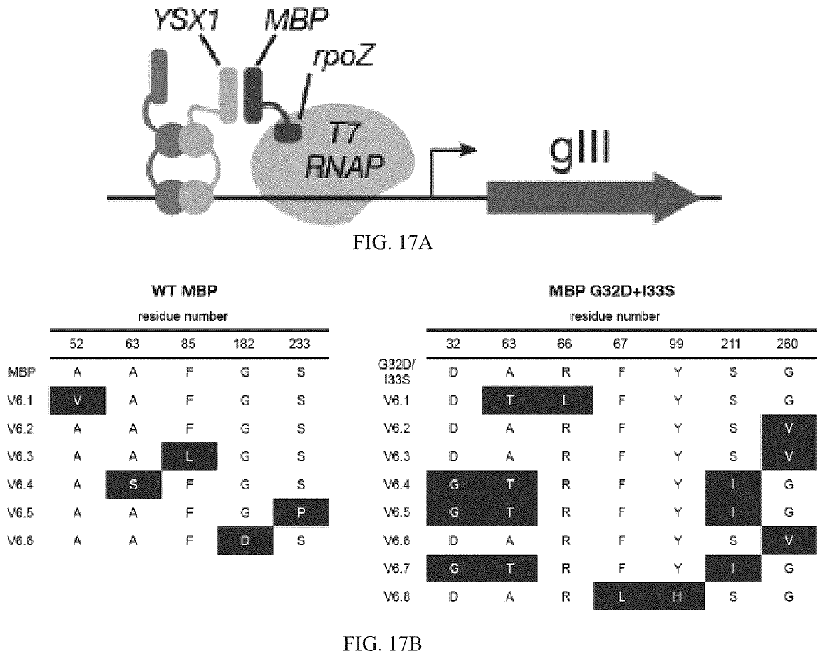

To facilitate and automate the liquid handling needs of PACE in eVOLVER, a customized “millifluidic” integrated peristaltic pumps (IPPs), inspired by integrated microfluidics³⁵, was developed that can be inexpensively manufactured using laser cutting to achieve accurate, tunable small volume flow rates (<0.1 to 40 μL/s) (FIGS. 15A-16D, see also Example 2). To test the evolutionary capabilities of ePACE, a folding-defective (G32D/133S) maltose-binding protein (MBP) variant validated in traditional PACE³⁰ was evolved. Previously, this folding defective MBP was evolved using a two-hybrid selection scheme to optimize both soluble expression of the MBP variant and binding to an anti-MBP monobody³⁰. This evolution was replicated using ePACE, yielding evolved MBP variants with mutations at residues clustered around the monobody-MBP interaction interface (D32G, A63T, R66L) that were previously observed in PACE (FIGS. 17A-17B)³⁰. These results demonstrated that eVOLVER equipped with IPP devices could successfully support and automate PACE, validating the ePACE platform for high-throughput continuous directed evolution.

### Development of a High-Throughput Base Editing-Dependent PAM Profiling Method

Next, a method to rapidly profile the PAM scope of Nme2Cas9 variants that emerge during evolution was developed. Assessing PAM compatibility by testing individual sites in mammalian cells is throughput-limited. Although many library-based PAM-profiling methods have been described, these methods rely on nuclease activity (PAM depletion¹², PAMDA^{14,18}, TXTL PAM profiling³⁶, CHAMP³⁷, etc.) or Cas protein binding activity (PAM-SCANR³⁸, CHAMP³⁷, etc.), which may not fully reflect PAM compatibility in precision gene editing applications such as base editing. A mammalian cell base editing profiling assay^{11,39} was previously developed; however, this method was both slower and costlier than cell-free^{36,37} or E. coli-based^{12,14,18,38} methods, making it better suited for the characterization of late-stage variants.

To address the need to rapidly assess the PAM specificities of newly evolved Cas9 variants in base editor form, a base editing-dependent PAM profiling assay (BE-PPA) was developed. In BE-PPA, a protospacer or library of protospacers containing target adenines (ABE-PPA) or cytosines (CBE-PPA) is installed upstream of a library of PAM sequences. (FIGS. 6A-6B). This library is transformed into E. coli along with a plasmid expressing a base editor of interest. The PAM profile observed for BE2 (rAPOBEC1-dSpCas9-UGI) using CBE-PPA closely matched (R²=0.97) the PAM profile observed for the related CBE. BE4, in mammalian HEK293T cells¹¹ (FIG. 6C, Table 1), validating BE-PPA as a rapid base editor PAM profiling method.

TABLE 1
CHOPCHOPv3 identified off-target sites

| | | | SEQ | | |
|---|---|---|---|---|---|
| | | | ID | | |
| | | | NO | PAM | Mismatches |
| | | Protospacer sequence | (top: | (top: | (PAM |
| | | (5′-3′; | Nme2Cas9, | Nme2Cas9; | proximal\hat{ }) |
| Site/Off- | | top: Nme2Cas9, | bottom: | bottom; | (top: |
| target | Gene | bottom: SpRY) | SpRY) | SpRY) | Nme2Cas9; |
| Site 1 | PDCDI | GCAGATCCCACAGGCGCGCTGGC | 32 | CAGTCG | bottom: |
| | | GATCCCACAGGCGCCCTGGC | 58 | CAG | SpRY) |
| Site1- | Intergenic | GCAttTCCCACAGGCGCCCTGGC | 59 | GATGCC | 2(0) |
| OT1 | | WTCCCACAGGCGCCCTGGC | 60 | GAT | 2(0) |
| Site 1- | PLCH2 | TgAGgTCCCACAGGCcCCCTGGC | 63 | GCAGCC | 4(1) |
| OT2 | | GgTCCCACAGGCcCCCTGGC | 64 | GCA | 2(1) |
| Site 1- | Intergenic | TggGcTCCCACAGGCcCCCTGGC | 65 | CTCCCC | 5(1) |
| OT3 | | GcTCCCACAGGCCCCCTGGC | 66 | CTC | 2(1) |
| Site 1- | TCF20 | AgAGATaCCACAGGCaCCCTGGC | 67 | ATGATA | 4(1) |
| OT4 | | GATaCCACAGGCaCCCTGGC | 68 | ATG | 2(1) |
| Site 1- | CXXC5 | GCtGcTCCCAgAGGCGCCCTGGC | 69 | TCTGCA | 3(0) |
| OT5 | | GcTCCCAgAGGCGCCCTGGC | 70 | TCT | 2(0) |
| Site 1- | Intergenic | agAGATCaCcCAGGCGCCCTGGC | 71 | TCATGC | 4(0) |
| OT6* | | GATCaCcCAGGCGCCCTGGC | 72 | TCA | 2(0) |
| Site 1- | SAPCD2P | tggGAcCCCACAtGCGCCCTGGC | 73 | CGGGAC | 5(0) |
| OT7* | 4 | GAcCCCACAtGCGCCCTGGC | 74 | CGG | 2(0) |
| Site 2 | FANCF | GCTGCAGAAGGGATTCCATGAGG | 75 | TGCGCG | — |
| | | GCAGAAGGGATTCCATGAGG | 76 | TGC | |
| Site 2- | Cllorf80 | tgTGaAGAAGGGtTTCCATGAGG | 77 | AGATAC | 4(0) |
| OT1 | | GaAGAAGGGtTTCCATGAGG | 79 | AGA | 2(0) |
| Site 2- | ANO3 | GaTtCtGAAGGGATTCCATGAGG | 80 | TCTAAA | 3(0) |
| OT2 | | tCtGAAGGGATTCCATGAGG | 81 | TCT | 2(0) |
| Site 2- | MEAK7 | caTGgAGAAGGGATCCCATGAGG | 82 | ACAAGG | 4(1) |
| OT3 | | GgAGAAGGGATCCCATGAGG | 83 | ACA | 2(1) |
| Site 2- | Intergenic | GgTaCAGAAGGGeTTCCATGAGG | 84 | CTGGGT | 3(0) |
| OT4 | | aCAGAAGGGcTTCCATGAGG | 85 | CTG | 2(0) |
| Site 2- | SLC19AI | GgaGCAGAAGGCATTtCATGAGG | 86 | GGTCAA | 4(1) |
| OT5 | | GCAGAAGGCATTtCATGAGG | 87 | GGT | 2(1) |
| Site 2- | LOC1053 | cagGCAGAAaGGAaTCCATGAGG | 88 | ACTCCC | 5(1) |
| OT6 | 74492 | GCAGAAaGGAaTCCATGAGG | 89 | ACT | 2(1) |
| Site 2- | MAP9 | aagGCAGAtGGGATTCCtTGAGG | 90 | TCTGGA | 5(1) |
| OT7 | | GCAGAtGGGATTCCtTGAGG | 91 | TCT | 2(1) |
| Site 2- | Intergenic | aagGCAGAAGGaATTCCATGAGt | 92 | TAAAGT | 5(1) |
| OT8 | | GCAGAAGGaATTCCATGAGt | 93 | TAA | 2(1) |
| Site 2- | RIPOR2 | ctTGCtGAAGGGATTCaATGAGG | 94 | TGCTAC | 4(1) |
| OT9 | | GCtGAAGGGATTCaATGAGG | 95 | TGC | 2(1) |
| Site 2- | Intergenic | GagGCAGgAGGGATTCCCTGAGG | 96 | AAGTGA | 4(1) |
| OT10 | | GCAGgAGGGATTCCCTGAGG | 97 | AAG | 2(1) |
| Site 2- | Intergenic | agaGCgGAAGGGATgCCATGAGG | 98 | GATCCG | 5(1) |
| OT11 | | GCgGAAGGGATgCCATGAGG | 99 | GAT | 2(1) |
| Site 2- | Intergenic | aaaGgAGAAGGaATTCCATGAGG | 119 | ATACAA | 5(0) |
| OT12* | | GgAGAAGGaATTCCATGAGG | 120 | ATA | 2(0) |

*site did not sequence well (mixed bases) with HTS primers that were tried. These sites were subsequently excluded from downstream analysis.
\hat{ }PAM proximal defined as positions within 10 bases of the PAM

### Strategy for Evolving the PAM Scope of Nme2Cas9

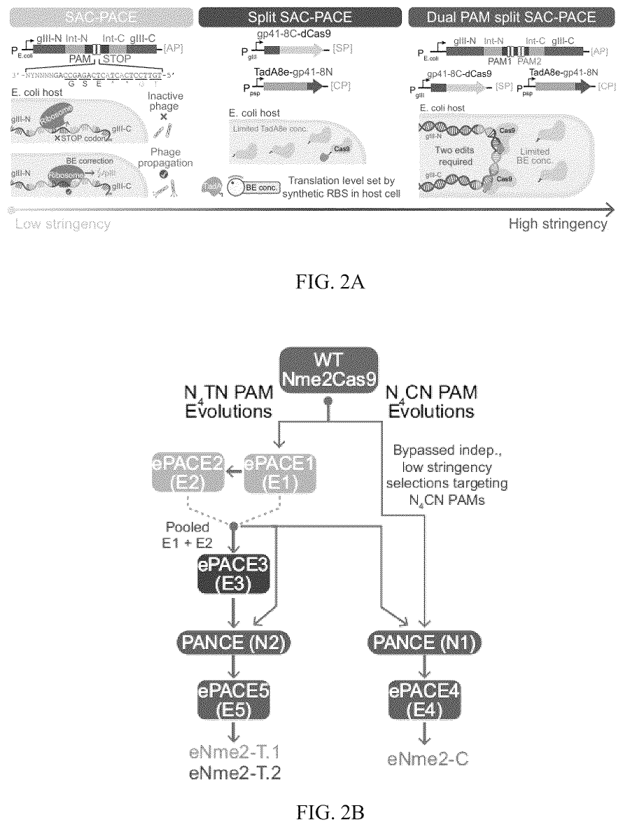

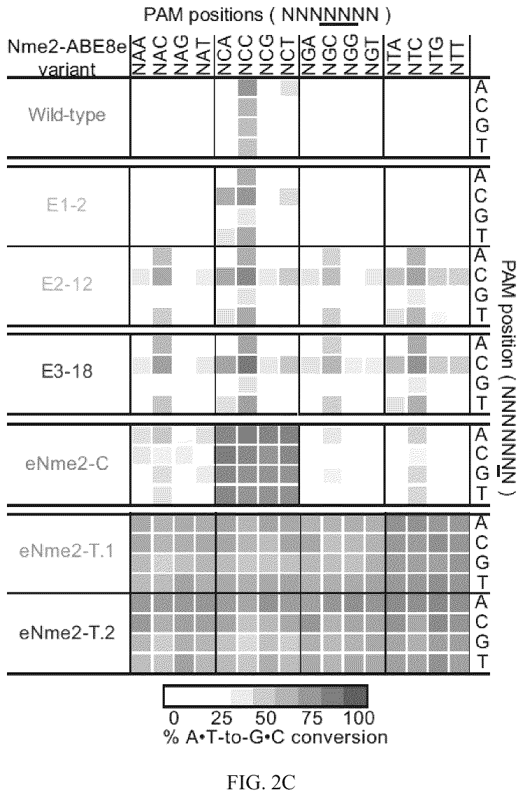

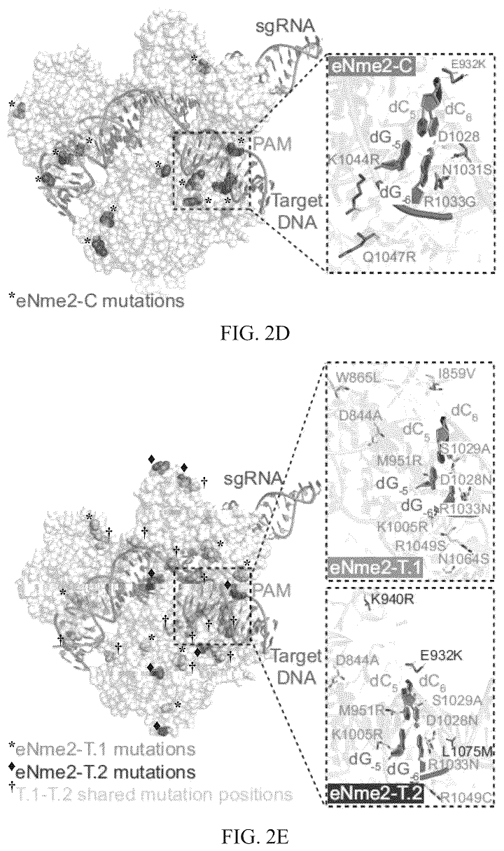

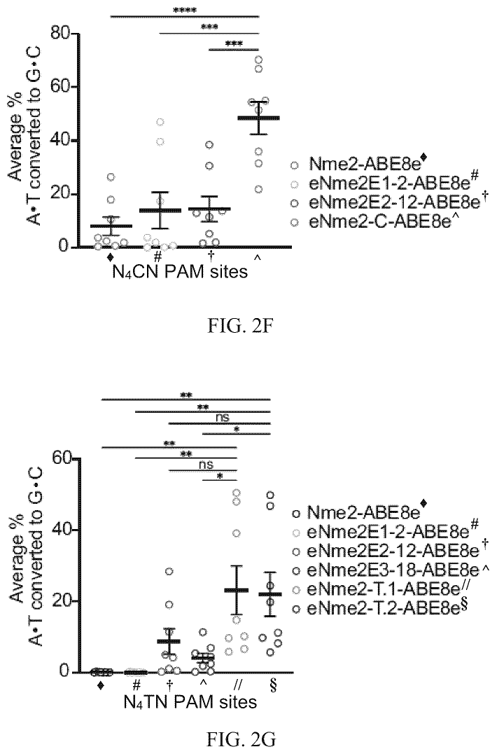

Having validated the SAC-PACE selection, the ePACE system for high-throughput continuous evolution, and the BE-PPA method for profiling PAM compatibility of base editors, the next step was identifying a desirable target PAMs for evolving Nme2Cas9. In overnight propagation assays, phage containing Nme2-ABE8e exhibited modest to strong propagation (N₃NCG<N₃NCA<N₃NCT<N₃NCC) on the set of 16 N₃NCN PAMs, and strong propagation on N₃NTC PAMs if the base immediately downstream of the canonical six base pair PAM was a C (PAM position 7, NNNNNNN, counting the canonical PAM as positions 1-6), likely due to PAM slippage (FIG. 1E)⁴⁰. This initial activity suggested an overall evolution campaign along two trajectories (FIG. 2B): a more difficult trajectory towards activity on N₄TN PAMs that could require several selection stringencies, and a simpler trajectory towards N₄CN-active variants. If successful, these variants could together enable targeting of PAM sequences largely complementary to the PAM scope of existing, high-activity SpCas9 variants.

### Low Stringency Evolution of Nme2Cas9 Towards N₄TN PAM Sequences

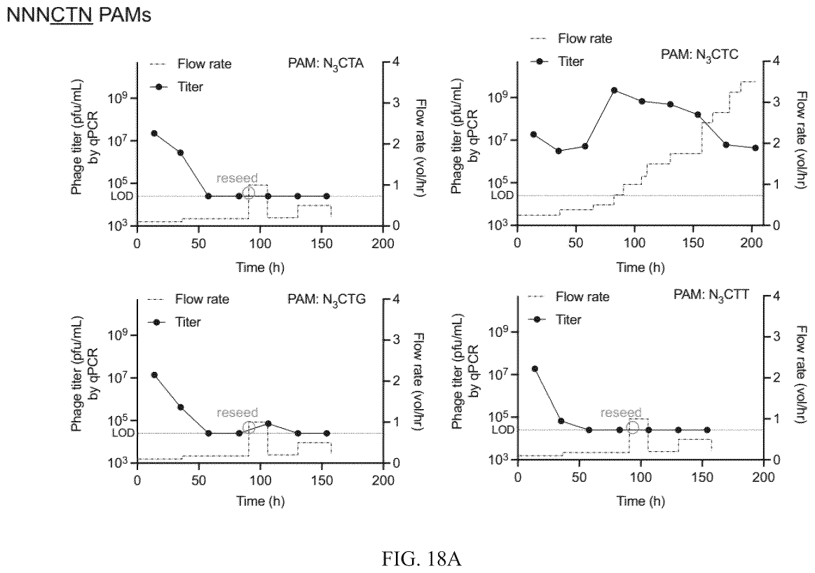

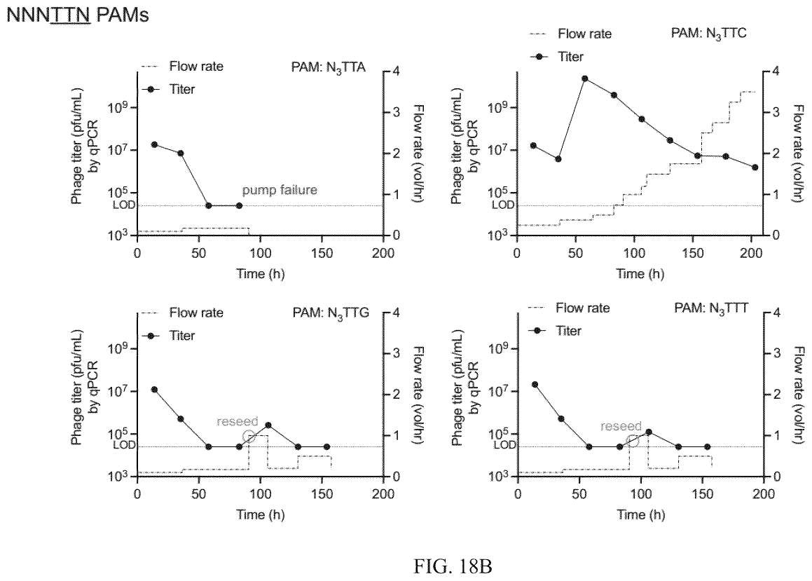

The evolution platform was used to perform parallel SAC-PACE selections to evolve Nme2Cas9 variants towards specific N₄TN PAM sequences (FIG. 2A-2G). The initial activity of wild-type Nme2Cas9 was used on some N₄TTC PAMs (FIG. 1D) as an evolutionary stepping-stone to access other N₄TN PAMs. Using the original (low stringency) SAC-PACE selection featuring one protospacer, two stop codons, and one target PAM (FIG. 2A, left panel), a wild-type Nme2-ABE8e was evolved on host cells containing APs with each of the eight possible N₃YTN APs and the mutagenesis plasmid (MP6)⁴ (ePACE1, FIG. 2B). As expected, all APs aside from those containing a N₃TTC or N₃CTC PAM washed out rapidly. However, those two PAM-containing lagoons persisted at up to 2 volumes/hour and yielded Nme2Cas9 variants with PAM-dependent mutational convergence (FIGS. 18A-19A). Consensus mutations occurred both inside (11025S, R1033K, S1043R for CTC PAM variants, Y1035C/H for TTC PAM variants) and outside of the PID (Y441C, K581R, D844V/G for CTC PAM variants; I462V, N616S, D844V for TTC PAM variants), suggesting potential PAM-specific and PAM-independent improvements to Nme2Cas9. Indeed, early evolved variants (e.g. E1-2-ABE8e) supported base editing activity on non-canonical PAMs and improved activity on wild-type N₄CC PAMs in human cells (FIG. 19B). Expanded PAM activity appeared strongest on N₄CN PAMs and was minimal on N₄TN PAMs.

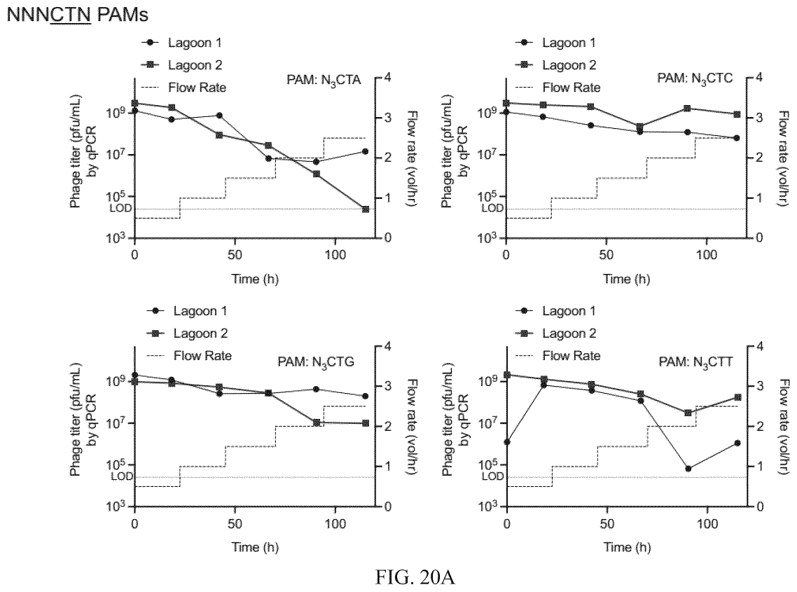

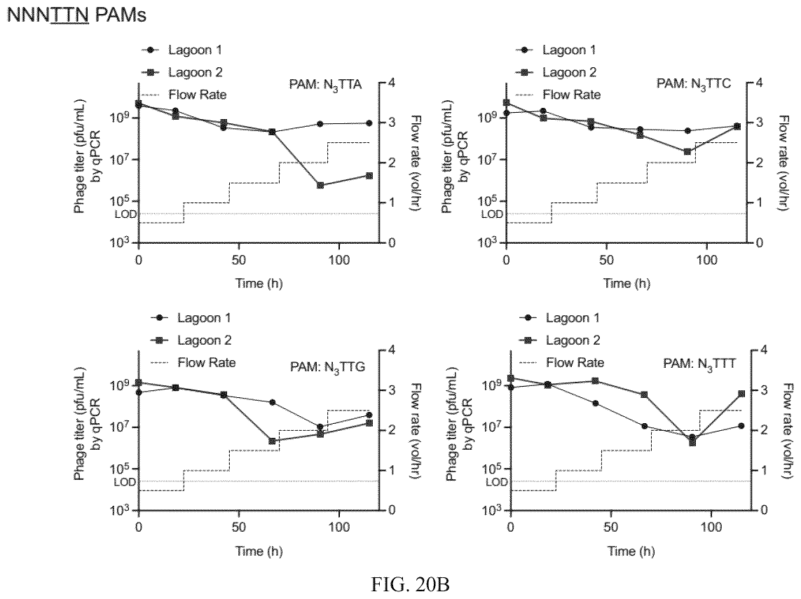

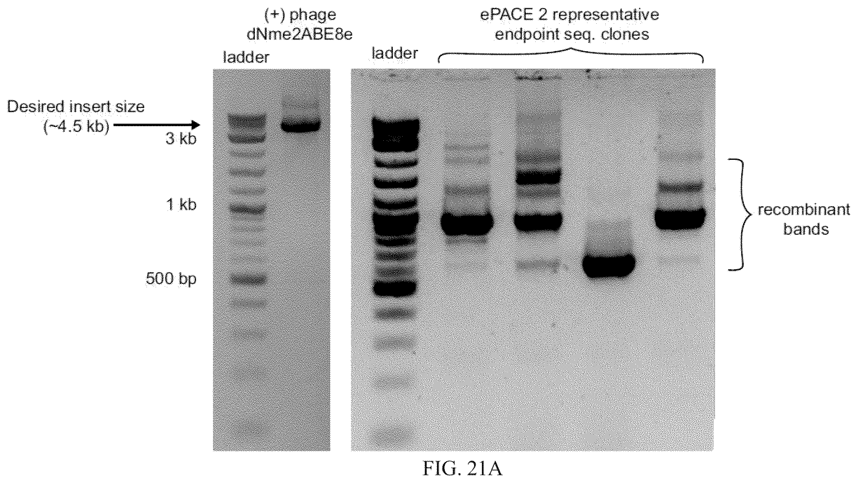

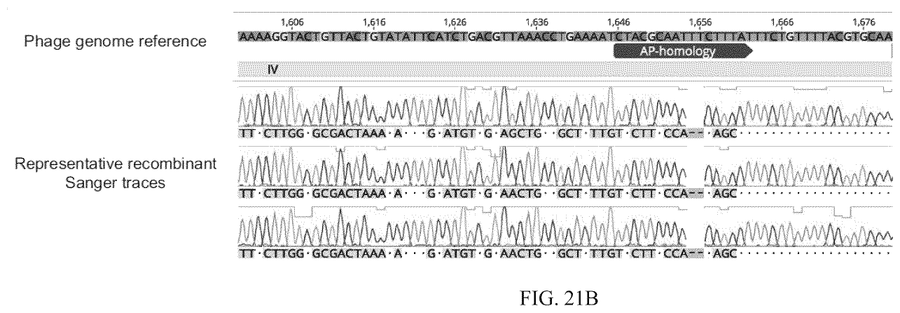

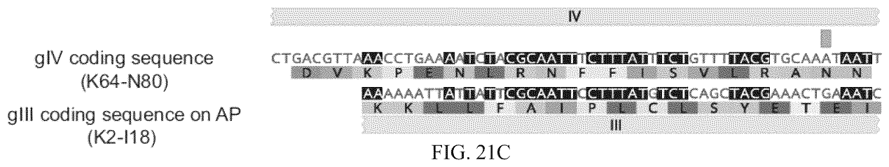

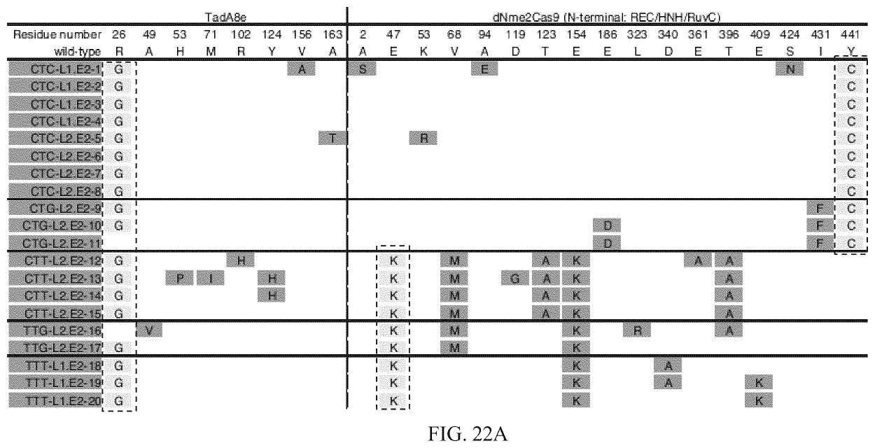

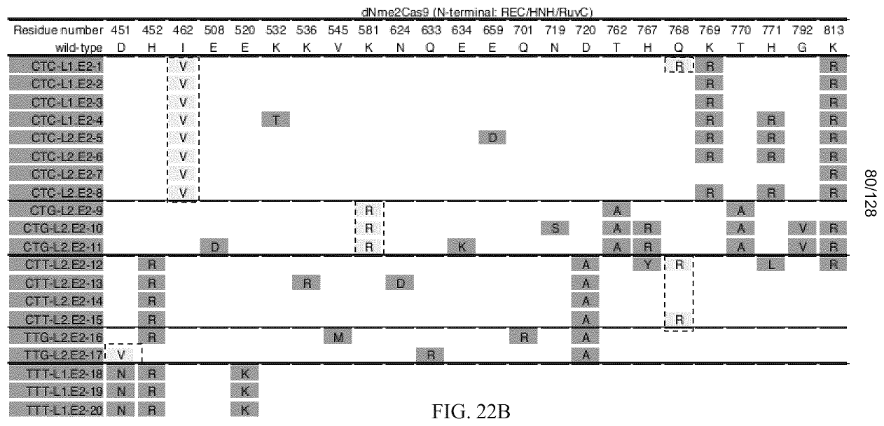

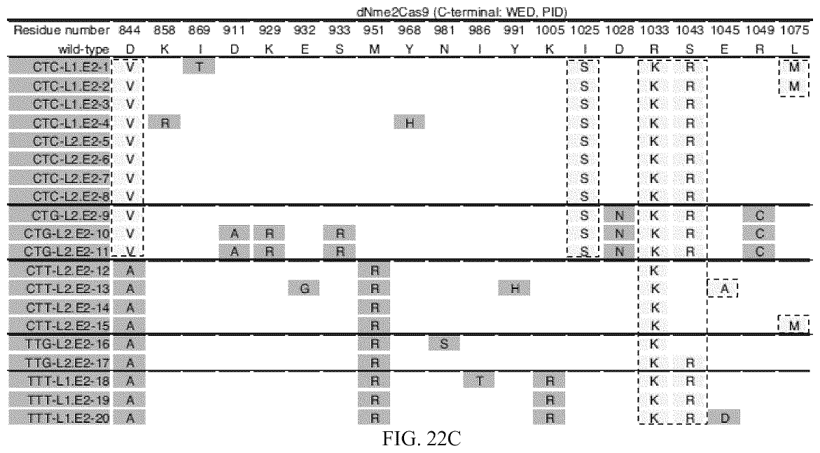

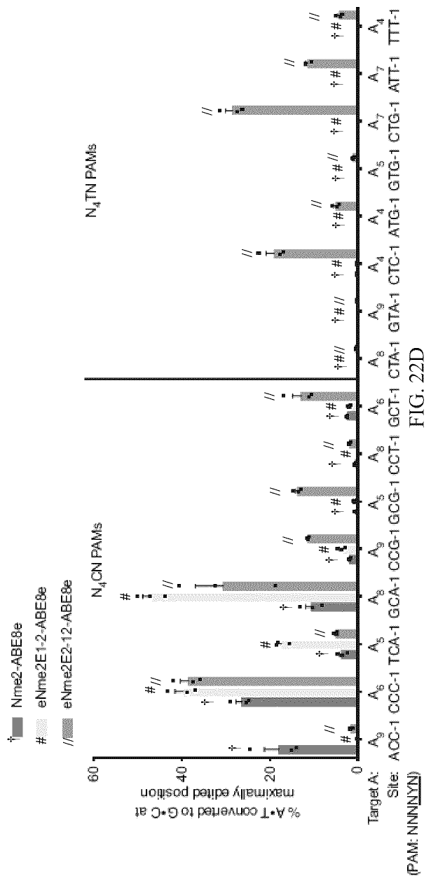

All PAM lagoons were reseeded with pooled phage from the two surviving PAMs (ePACE2) (FIG. 2B). All lagoons exhibited strong propagation at up to 2.5 volumes/hour (FIGS. 20A-20B), but surviving phage appeared to lose the Nme2-ABE8e cassette, indicating recombination to bypass the selection (FIGS. 21A-21C, Example 2). The clones that did not show recombination were sequenced and novel mutations were found that, again, appeared to cluster by PAM/lagoon both in and outside of the PID (FIG. 22A). In mammalian cells, while expanded PAM compatibility did extend to some N₄TN PAMs, activity appeared to be site-dependent while moderate activity on N₄CN PAMs was retained (FIG. 22D). These ePACE1 and ePACE2 outcomes suggested that the low stringency SAC-PACE selection may be insufficient to generate highly active Nme2Cas9 PAM variants.

The ABE-PPA was used to profile the PAM compatibility of wild-type Nme2-ABE8e and a representative ABE variant from both ePACE1 (E1-2-ABE8e) and ePACE2 (E2-12-ABE8e) that had exhibited improved mammalian cell base editing activity on N₄YN PAMs (FIGS. 2C, 6D, and 6E, Table 1). While both evolved variants exhibited improved activity on N₄CD (where D=A, G, or T) PAMs over Nme2-ABE8e (17%, 23%, and 32% average A•T-to-G•C conversion for Nme2-ABE8e, E1-2-ABE8e, and E2-12-ABE8e, respectively), only the more evolved variant, E2-12-ABE8e, exhibited improved N₄TN PAM activity (2%, 2%, and 39% average A•T-to-G•C conversion for Nme2-ABE8e, E1-2-ABE8e, and E2-12-ABE8e, respectively). This result suggests a model in which broadened activity on N₄CN PAMs precedes activity on N₄TN PAMs.

Further examination of the ABE-PPA data indicated that broadened PAM activity of early evolved Nme2Cas9 variants was primarily driven by an acquired C preference at the undesired PAM position 7, a position not recognized by the wild-type enzyme⁴². While E1-2-ABE8e and E2-12-ABE8e progressively improved base editing activity compared to wild-type Nme2-ABE8e on N₄YNC PAM sites (18%, 29%, and 58% average A•T-to-G•C conversion for Nme2-ABE8e, E1-2-ABE8e, and E2-12-ABE8e, respectively), base editing activity was improved to a lesser extent at N₄YND PAM sites (14%, 14%, and 33% average A•T-to-G•C conversion for Nme2WT ABE8e, E1-2-ABE8e, and E2-12-ABE8e, respectively). This discrepancy suggested the need for higher selection stringency to restrict the survival of Cas variants that acquire expanded PAM recognition at undesired positions.

### Increasing SAC-PACE Selection Stringency to Evolve High-Activity Nme2Cas9 Variants

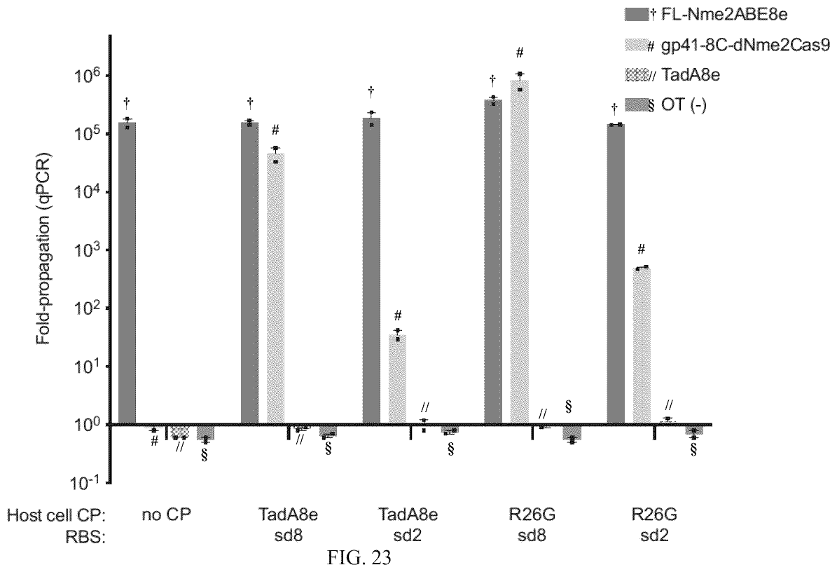

In previous efforts evolving SpCas9, restricting the amount of active enzyme and requiring additional PAM recognition via a multi-PAM system increased selection stringency and enabled evolution of higher activity variants¹¹. Therefore, similar strategies were implemented in SAC-PACE to evolve high-activity Nme2Cas9 variants while preventing selectivity at undesired PAM positions (FIG. 2A). To limit the amount of active base editor, a split-intein strategy was used with the base editor split at the linker between TadA8e and dNme2Cas9 (the double mutant Nme2Cas9 containing D16A and H588A mutations), which could tolerate the insertion of an extein scar (split SAC-PACE) (FIG. 2A, middle panel). The fast-splicing gp41-8 intein pair^{43,44} was selected as the Npu intein pair was already in use in the AP. In overnight propagation assays, only host cells containing a psp-driven TadA8e-gp41-8N construct on a complementary plasmid (CP) enabled survival of SP expressing gp41-8C-dNme2Cas9 (FIG. 23, see also Example 2). Since the expression level of the TadA8e construct could be controlled on the CP, this result validated the ability of the split SAC-PACE selection to limit base editor concentrations while continuing to select for evolving Cas9-containing SP.

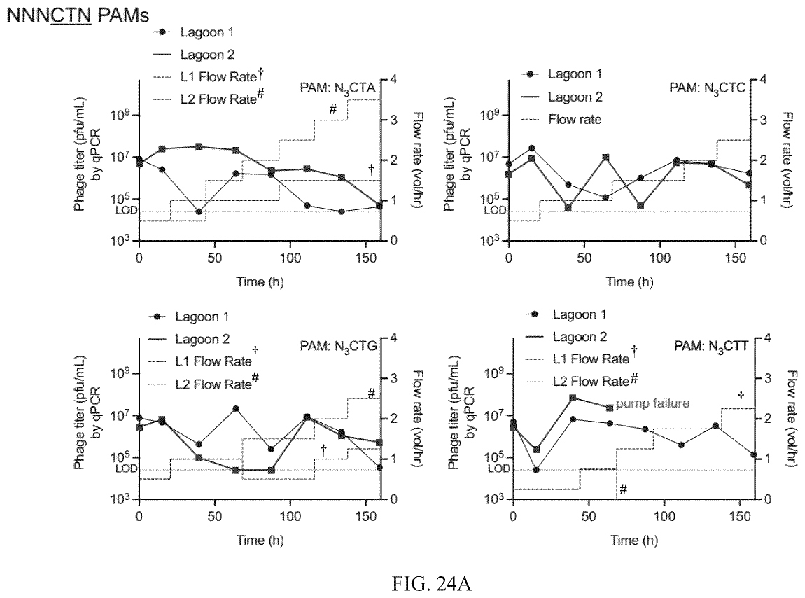

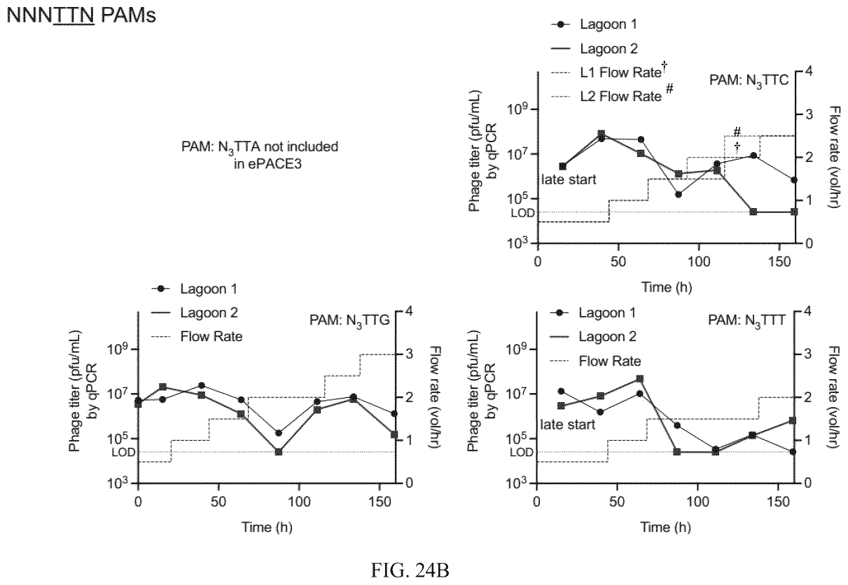

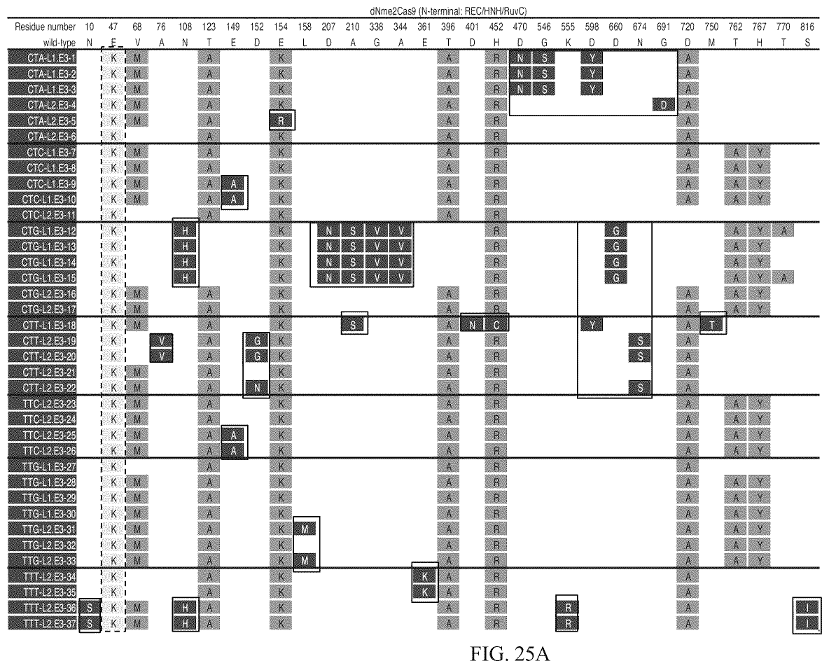

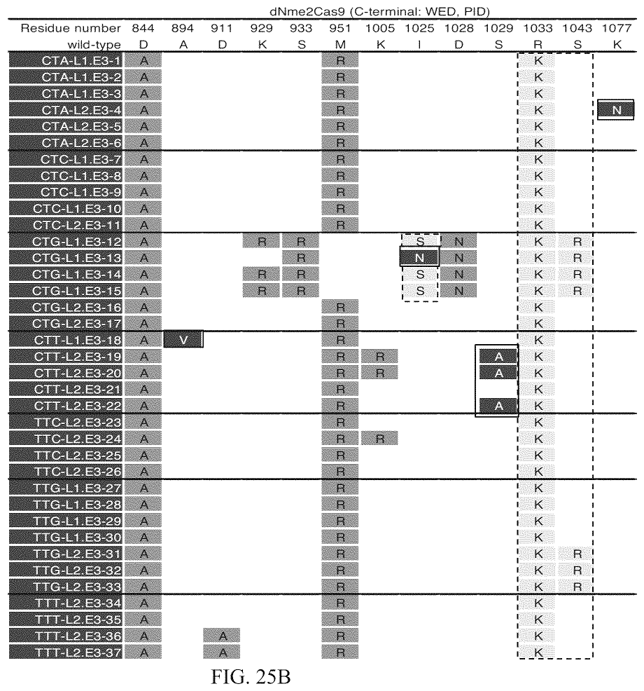

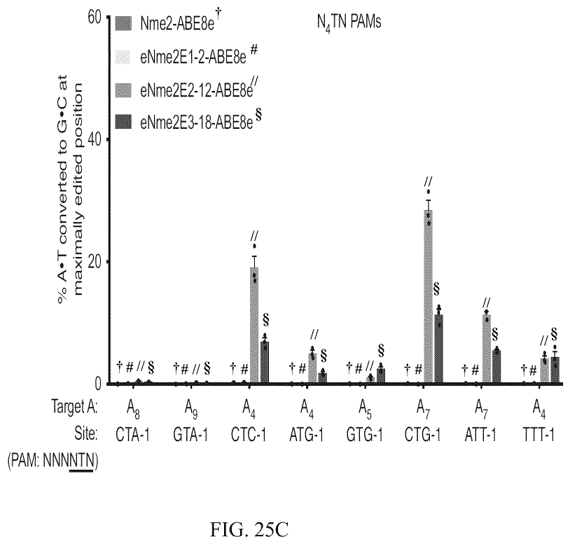

Using the intermediate-stringency split SAC-PACE selection, Nme2Cas9 variants that had emerged from low-stringency selections were further evolved. Endpoint phage from ePACE1 and ePACE2 were pooled, cloned into the split SP architecture, and then the SP was seeded into the split SAC-PACE selection (ePACE3) (FIG. 2B). All targeted PAMs exhibited moderate phage persistence (>10⁵ titers) within at least one lagoon at or above 2 vol/hour (FIGS. 24A-24B). Sequenced clones from lagoons other than the one targeting an N₃CT PAM showed very strong mutational convergence across lagoons and PAMs, suggesting that the resulting Nme2Cas9 variants likely were not acquiring PAM specificity at the positions defined in the evolutions (PAM positions 4 and 6) (FIGS. 25A-25B). ABE-PPE profiling of a representative variant from ePACE3 (E3-18-ABE8e) that had exhibited activity on N₄TN PAM sites in mammalian cells (FIG. 25C) showed comparable activity (31% and 39% average A•T-to-G•C conversion on N₄CD and N₄TN PAM sites, respectively) to the earlier evolved E2-12-ABE8e variant. However, this broadened PAM compatibility was again accompanied by a PAM position 7 C preference (61% vs. 33% average A•T-to-G•C conversion on N₄YNC and N₄YND PAM sites, respectively) (FIG. 6E), indicating that restricting enzyme concentration alone is insufficient to evolve higher activity variants with desired PAM preferences.

Thus, another layer of stringency control was added to increase the likelihood of evolving higher activity variants. A multiplexed-PAM selection requiring correction of a stop codon in two protospacers flanked by PAM sequences with alternate sequence identity at PAM positions 1-3 and 7 (NNNNNNN) was implemented, thereby forcing evolving Nme2Cas9 variants to recognize multiple nucleotides at undesired PAM positions. This selection was coupled with split SAC-PACE to produce a third (high stringency) scheme that was termed dual-PAM split SAC-PACE (FIG. 2B, right panel). With these developments, high-stringency evolution along both trajectories (N₄CN and N₄TN PAM sequences).

### High Stringency Evolution of Nme2Cas9 Towards N₄CN PAM Sequences

The outcomes of ePACE1 and ePACE2 revealed that improved activity on N₄TN PAMs was accompanied by broadened activity on N₄CN PAMs. It was determined that the mutational diversity from these evolutions could provide useful starting points for the evolution of N₄CN PAM compatibility. A trajectory was thus pursued with both wild-type Nme2Cas9 and pooled ePACE1 and ePACE2 (E1+E2) phage, subjecting these starting points to high stringency evolutions in parallel via dual PAM split SAC-PACE (FIG. 2B).

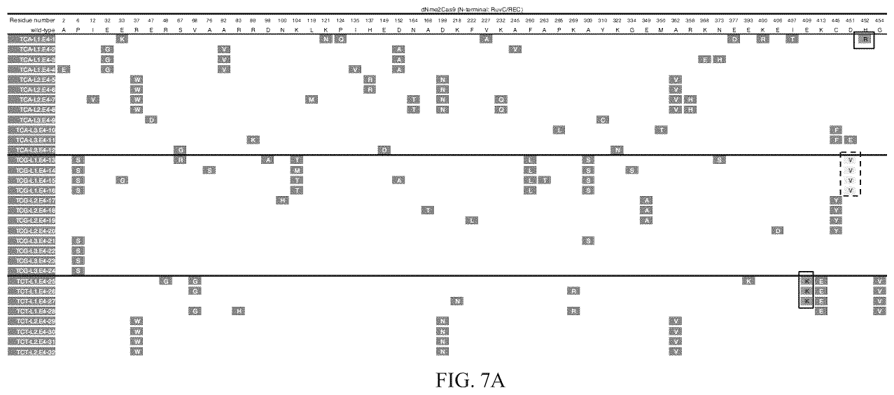

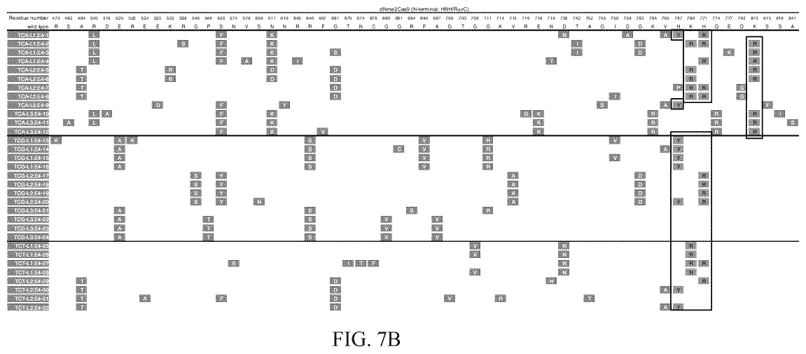

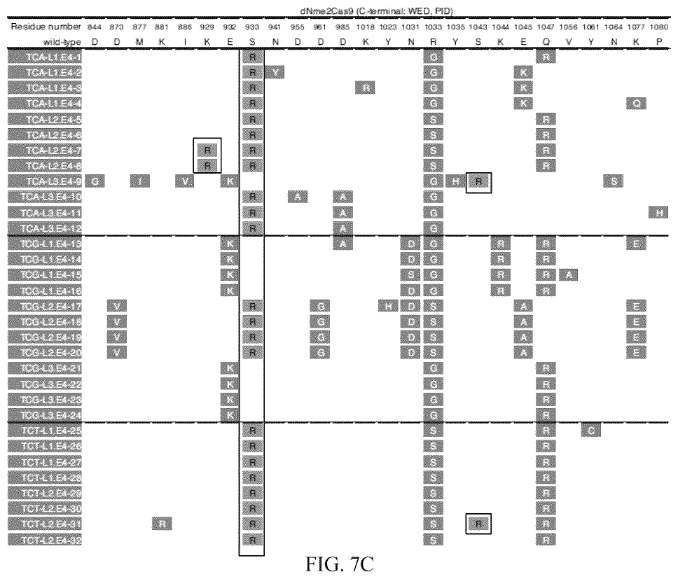

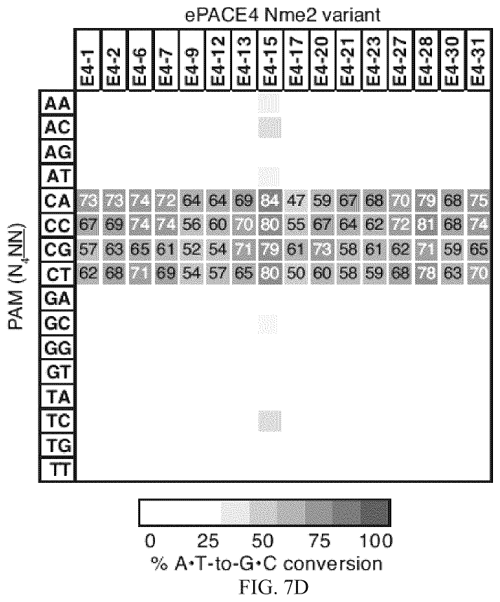

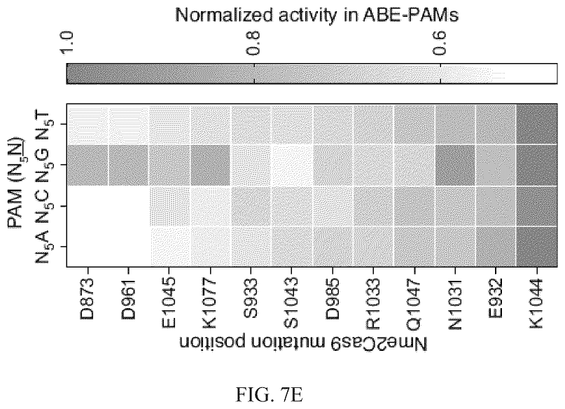

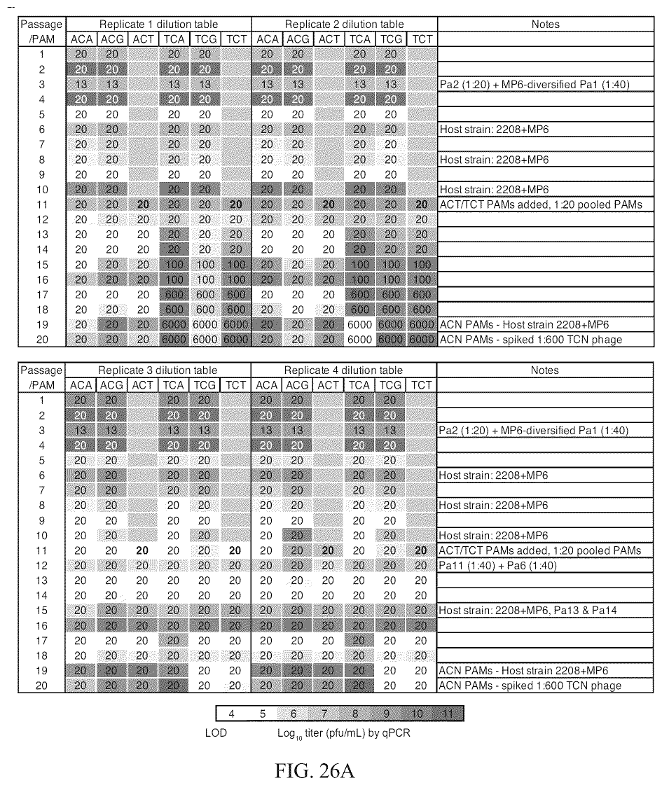

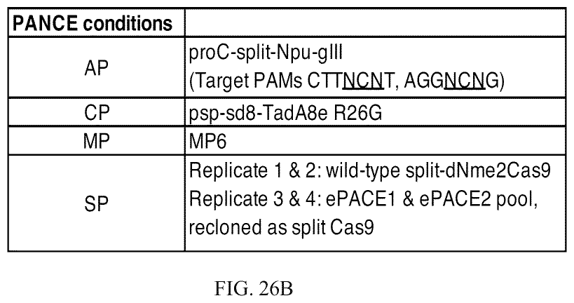

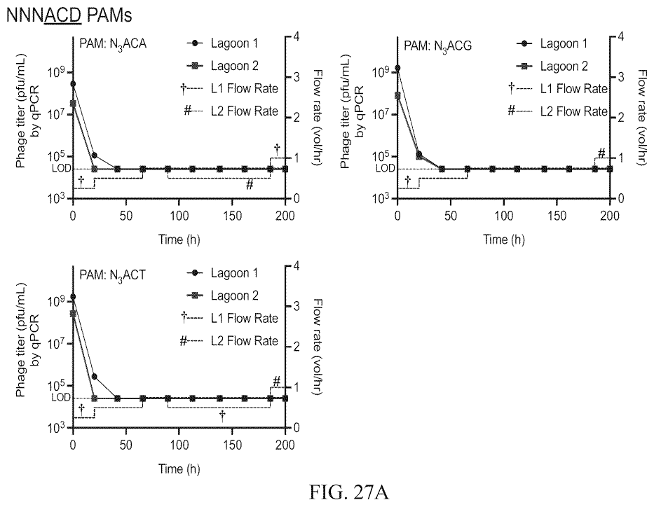

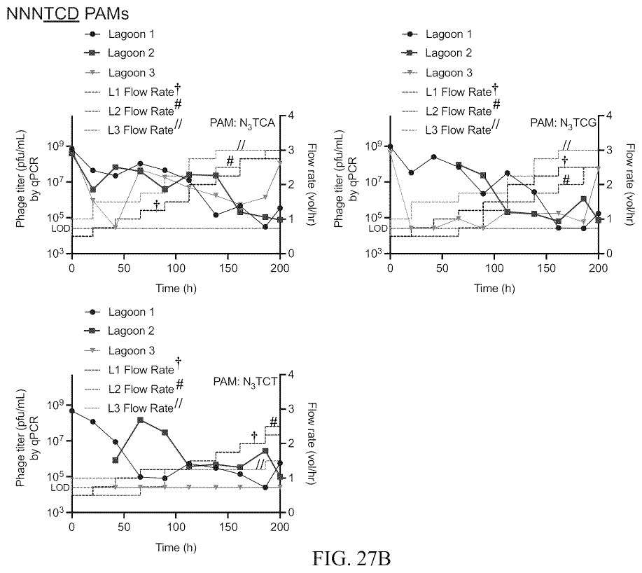

SP containing either wild-type or E1+E2 phage propagated insufficiently for PACE on N₄CN-containing APs requiring dual edits. Therefore, evolution was started with PANCE, a non-continuous version of PACE in which phage are discretely passaged following an incubation period (typically overnight)₃. Using PANCE (N1), either wild-type gp41-8C-dNme2Cas9 or pooled E1+E2 endpoint phage on the set of six N₃WCD (where W=A or T) PAMs was evolved (FIGS. 2B and 26A-26B). Following 20 passages in PANCE, only some of the lagoons targeting N₃TCD PAMs appeared to consistently propagate. Phage from these lagoons were then seeded into ePACE (ePACE4) (FIGS. 2B and 27A-27B). Interestingly, few mutations from E1+E2 were retained in ePACE4, both within and outside the PID, suggesting evolution of a distinct mode of PAM recognition among ePACE4 clones (FIGS. 7A-7C).

Sixteen ePACE4 clones assayed using ABE-PPA exhibited strong and general ABE activity, averaging 66% editing across all N₄CN PAMs (FIG. 7D, Table 1). The E4-15 variant in particular, which was denoted as eNme2-C(SEQ ID NO: 1)(Nme2Cas9 P6S, E33G, K104T, D152A, F260L, A263T, A303S, D451V, E520A, R646S, F696V, G711R, 1758V, H767Y, E932K, N103IS, R1033G, K1044R, Q1047R, V1056A), achieved ≥80% A•T-to-G•C editing at all N₄CN PAM sites as an ABE8e, corresponding to a 4.8-fold average improvement in activity on N₄CT PAM sites over Nme2-ABE8e, and a 1.3-fold average improvement in activity even on N₄CC PAM sites natively recognized by wild-type Nme2Cas9 (FIGS. 2C-2D). Notably, activity improvements of ePACE4 variants on specific N₄CN PAMs appeared to be largely agnostic of the specific PAM offered during evolution, with most variants preferring N₄CA>N₄CC>N₄CT>N₄CG (FIGS. 7D-7E, Supplementary Note in Example 2). Importantly, ePACE4 variants (e.g. eNme2-C(SEQ ID NO: 1), FIG. 2C) no longer exhibited the preference for a C at PAM position 7 exhibited in earlier evolved variants. Collectively, these findings established that by requiring multiple PAM engagements, the dual PAM split SAC-PACE selection could successfully generate high-activity Cas9 variants with broadened PAM scope.

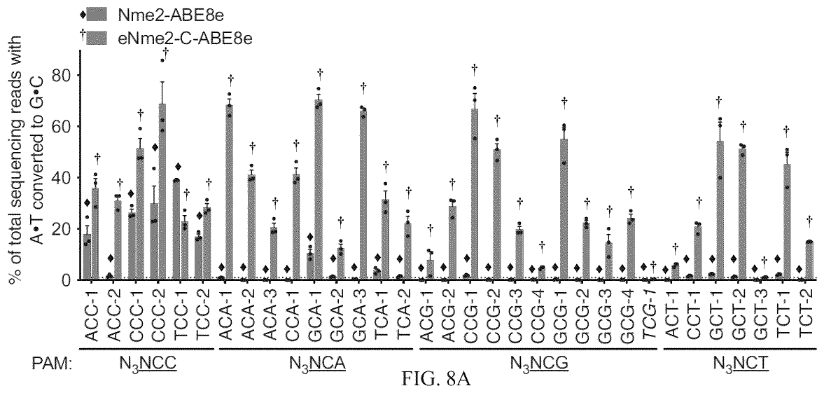

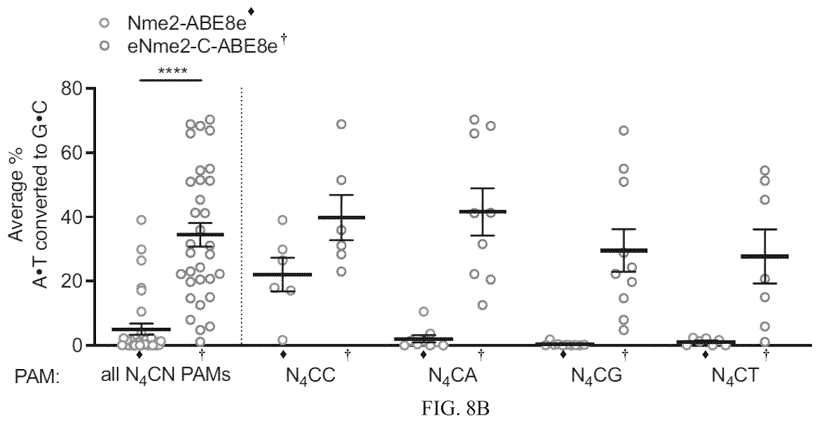

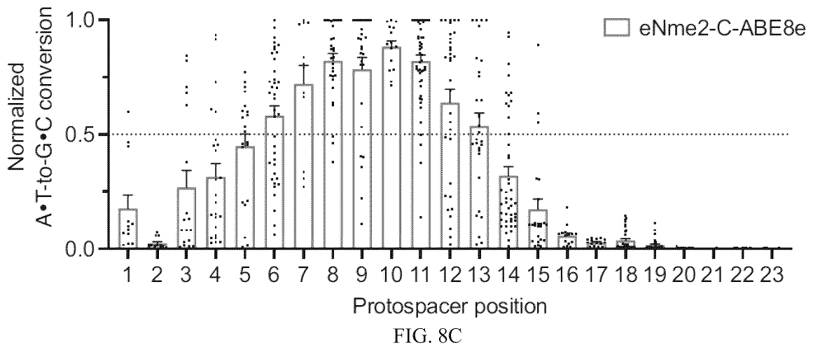

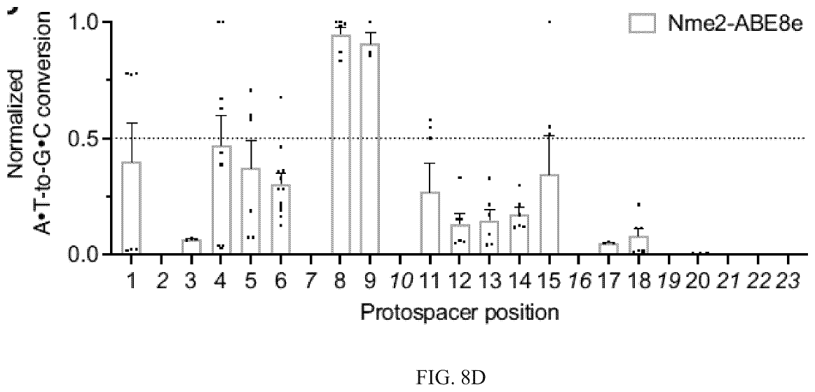

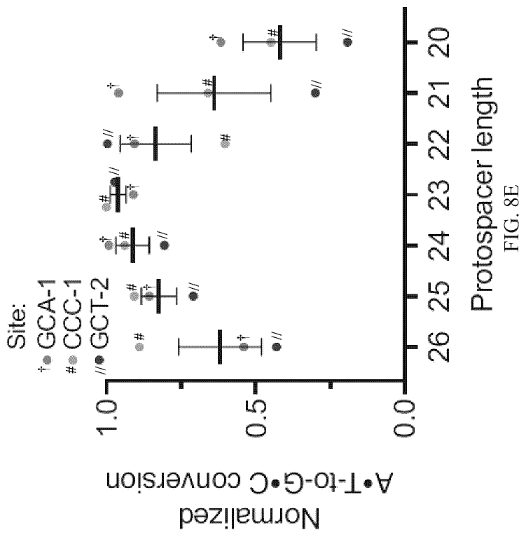

Encouraged by the PAM profile of ePACE4 variants, it was assessed whether the activity observed in bacterial cells successfully translated to mammalian cells. In HEK293T cells a robust ABE activity for eNme2-C-ABE8e was observed across all eight endogenous human genomic N₄CN sites previously tested. Notably, eNme2-C-ABE8e showed 2.0-fold higher average editing efficiency on N₄CC PAM sites and 15-fold higher editing efficiency on N₄CD PAM sites than Nme2-ABE8e, and 2.3 to 3.3-fold improved editing at all sites compared to earlier evolved variants eNme2-E1-2-ABE8e and eNme2-E2-12-ABE8e, respectively (FIG. 2E). To further test the N₄CN PAM generality of eNme2-C-ABE8e, the activity was evaluated at an additional 25 genomic sites flanked by N₄CN PAMs (for a total of 33 endogenous genomic sites tested) and an average of 34% A•T-to-G•C conversion was observed at the tested sites exhibiting base editing above 1% (32 of 33 sites), a 1.8- and 30-fold average improvement at N₄CC and N₄CD PAM sites, respectively, over Nme2-ABE8e (FIGS. 8A-8B). The editing window of eNme2-C-ABE8e is approximately between protospacer positions 9 and 16 (counting the PAM as positions 24-29) and was similar to the editing window of eNme2-ABE8e (FIG. 8C). Like Nme2Cas9, eNme2-C(SEQ ID NO: 1)retains a protospacer preference centered around 23 base pairs in length (FIGS. 8C-8E). As such, the editing window of eNme2-C-ABE8e was shown to be about 8 base pairs (bp). Together, the ABE-PPA data and the mammalian cell data suggest that eNme2-C-ABE8e is a robust adenine base editor that provides general access to N₄CN PAMs.

### High Stringency Evolution of Nme2Cas9 Towards N₄TN PAM Sequences

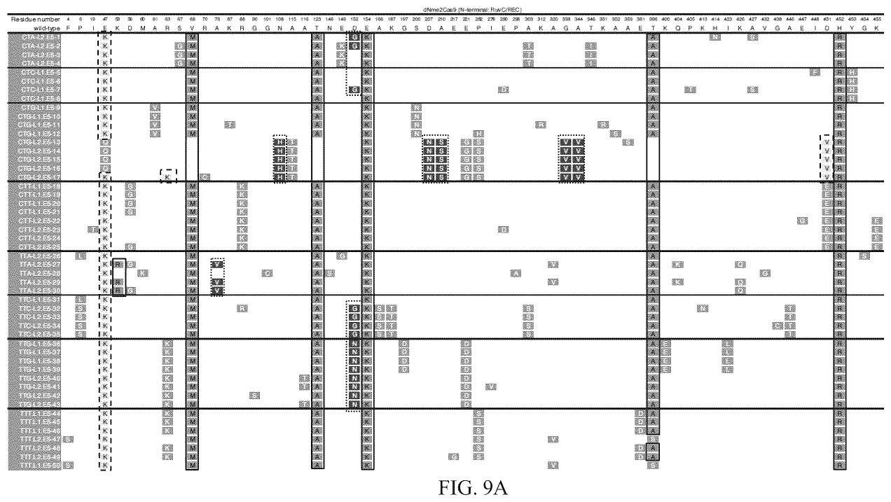

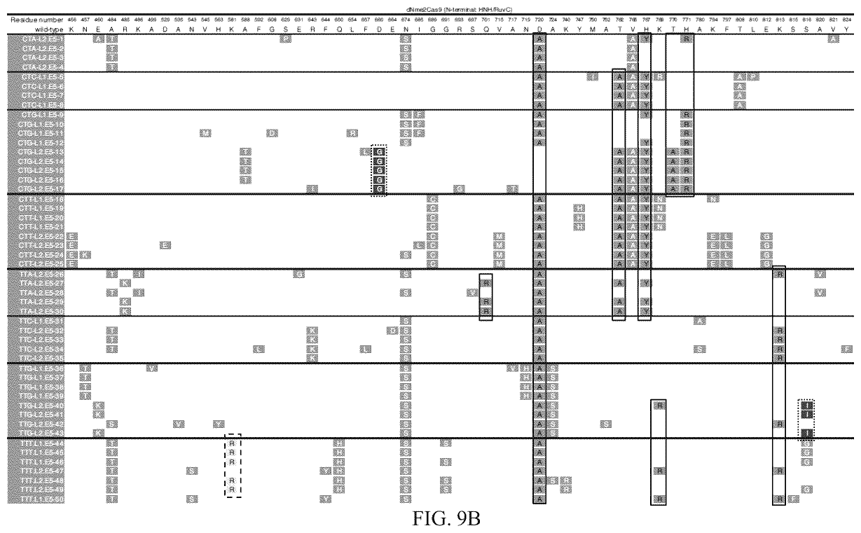

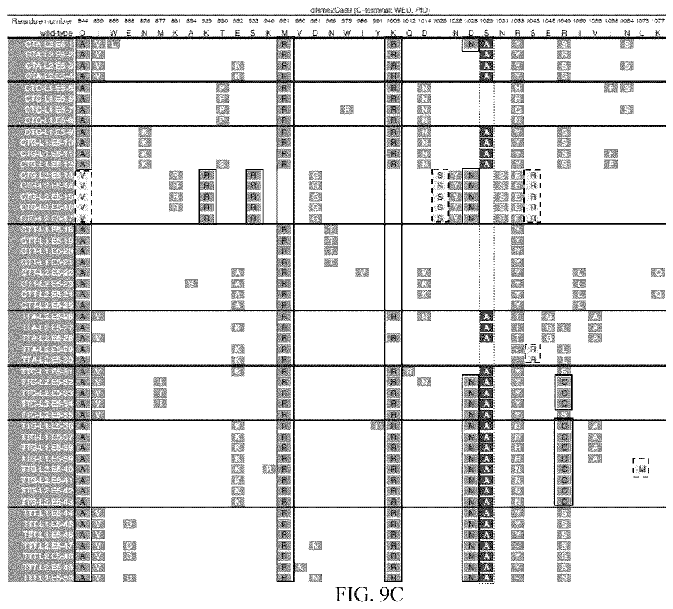

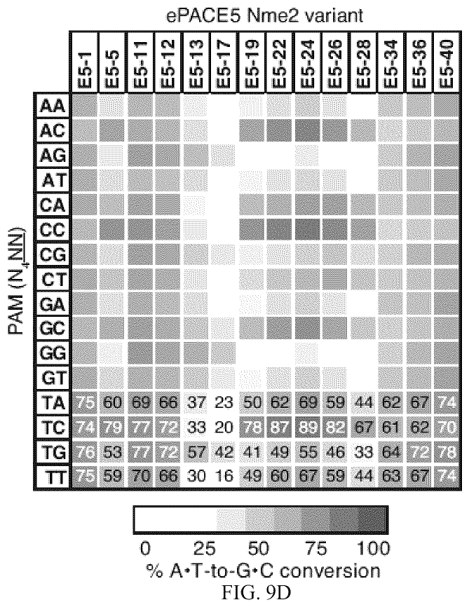

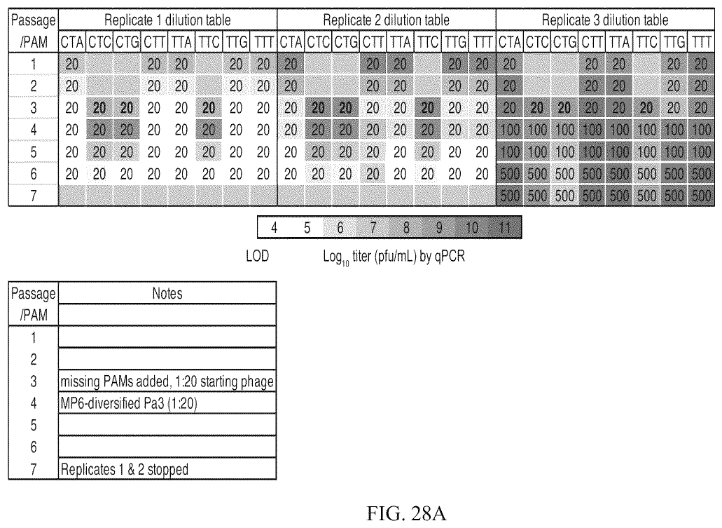

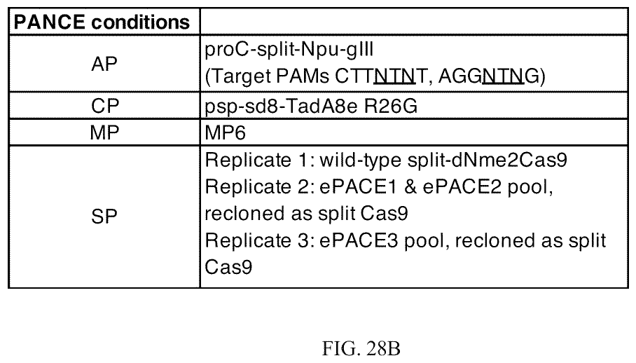

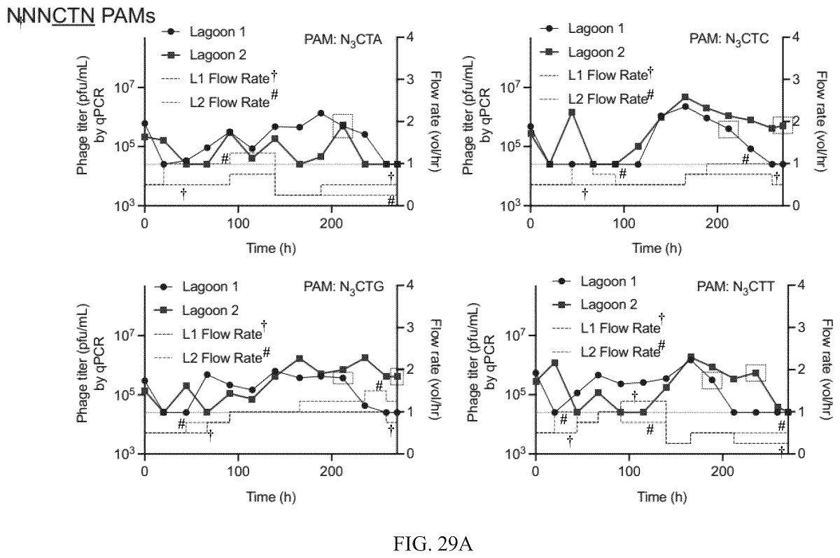

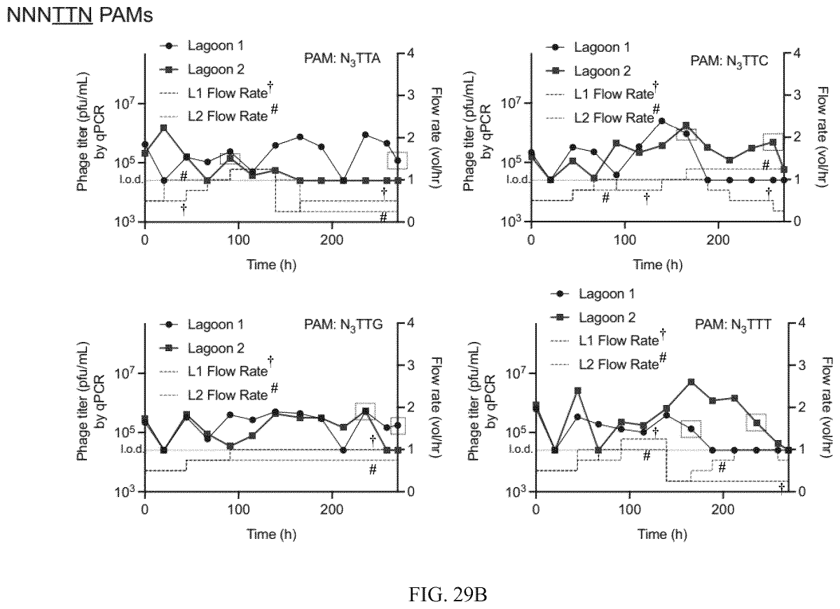

Following the success of the N₄CN trajectory using a high-stringency selection, the N₄TN trajectory was assessed using a similar approach. Starting with PANCE (N2), three different pools of MP6-diversified phage were evolved on each of the eight N₃YTN PAMs (FIGS. 2B and 28A-28B). Across eight PANCE passages, only lagoons seeded with ePACE3 endpoint phage propagated. These phage pools were subsequently seeded into ePACE (ePACE5). Under continuous evolution, these phage pools struggled to propagate, with phage washing out of many lagoons and only persisting with low titers (˜10⁵ pfu/mL) at low flow rates (<1.5 vol/hour) among surviving lagoons (FIGS. 29A-29B). Phage clones were sequenced from each lagoon at a timepoint during which titers exceeded 10⁵ pfu/mL. Most sequenced clones retained many of the strongly converged mutations from ePACE3, particularly in the non-PID region. However, in the PID, intra-lagoon convergence was observed at residue 1033 (which mediates the wild-type interaction with the PAM position 6 cytosine and previously converged to lysine in ePACE3) and residue 1049 (positioned proximal to the PAM) for lagoons evolved on the same PAM, but divergence across PAMs (R1033Y/E/N/H/T; R1049S/L/C), suggesting novel PAM-specific interactions at positions 4 or 6 made possible by the higher stringency selection (FIGS. 9A-9C).

Using ABE-PPA, it was observed that ePACE5 variants exhibited broad PAM compatibility (FIG. 9D, Table 1), in contrast to ePACE4 variants which exhibited strong N₄CN-specific activity. While N₄TN activity was the most enriched, substantial adenine base editing activity was observed at all other PAMs, which could increase downstream Cas-dependent off-target editing. Two clones, E5-1, which were denoted eNme2-T.1 (SEQ ID NO: 2) (Nme2Cas9 E47K, V68M, T123A, D152G, E154K, T396A, H413N, A427S, H452R, E460A, A484T, S629P, N674S, D720A, V765A, H767Y, H771R, V821 A, D844A, I859V, W865L, M951R, K1005R, D1028N, S1029A, R1033Y, R1049S, N1064S), and E5-40, which were denoted eNme2-T.2 (SEQ ID NO: 3) (Nme2Cas9 E47K, R63K, V68M, A116T, T123A, D152N, E154K, E221D, T396A, H452R, E460K, N674S, D720A, A724S, K769R, S816I, D844A, E932K, K940R, M951R, K1005R, D1028N, S1029A, R1033N, R1049C, L1075M), showed >70% average A•T-to-G•C editing across all N₄TN PAMs as ABE8e variants (FIGS. 2F and 9D). As with the ePACE4 variants, many ePACE5 variants no longer exhibited a preference at PAM position 7 (e.g. eNme2-T.1, eNme2-T.2, FIG. 2C), further highlighting the benefit provided by the multiplexed-PAM selection scheme.

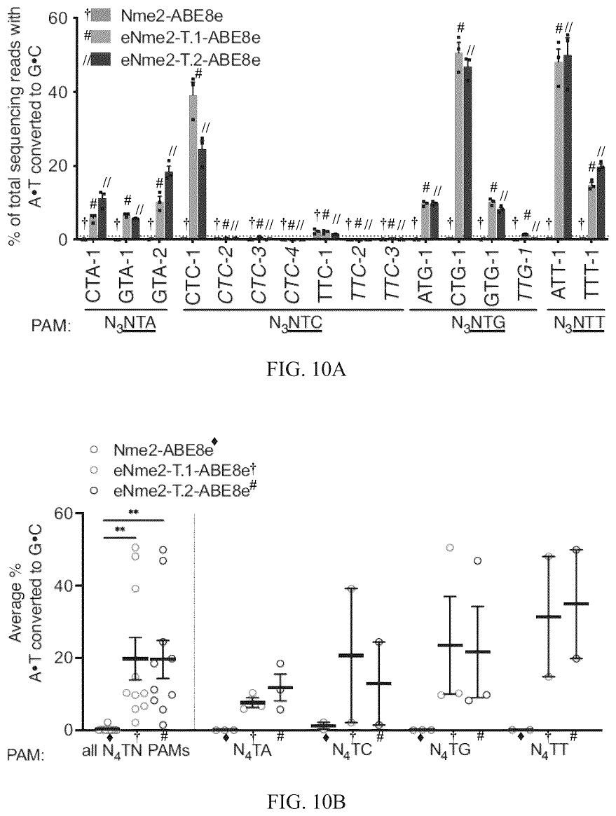

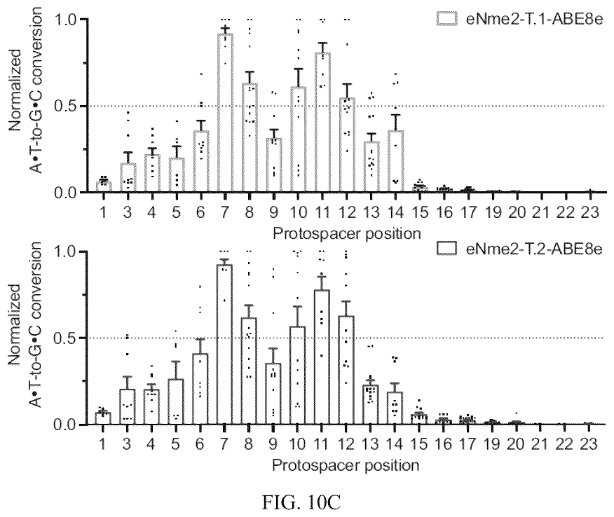

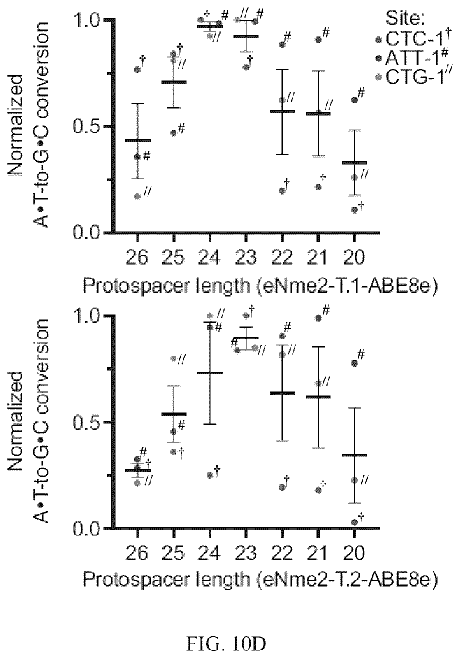

The eNme2-T.1 and eNme2-T.2 (SEQ ID NO: 3) variants were tested in HEK293T cells at the eight endogenous human genomic N₄TN sites previously tested. At these eight sites, eNme2-T.1-ABE8e and eNme2-T.2-ABE8e averaged 23% and 22% A•T-to-G•C editing, respectively, representing a 278- and 264-fold improvement in activity over wild-type Nme2-ABE8e (FIGS. 20 and 10A-10B). After including eight additional genomic N₄TN sites, eNme2-T.1-ABE8e and eNme2-T.2-ABE8e exhibited base editing efficiencies above 1% at 69% or 63% of the 16 total sites, respectively. Within the sites showing >1% base editing, efficiencies ranged from 1.4-51% for eNme2-T.1-ABE8e and from 1.4-50% for eNme2-T.2-ABE8e. Both variants appeared to have a slightly 5′ shifted base editing window compared to eNme2-C-ABE8e, between positions 7 and 12 of the protospacer (counting the PAM as positions 24-29), but showed similar protospacer length preferences of 23 base pairs (FIGS. 10C-10D).

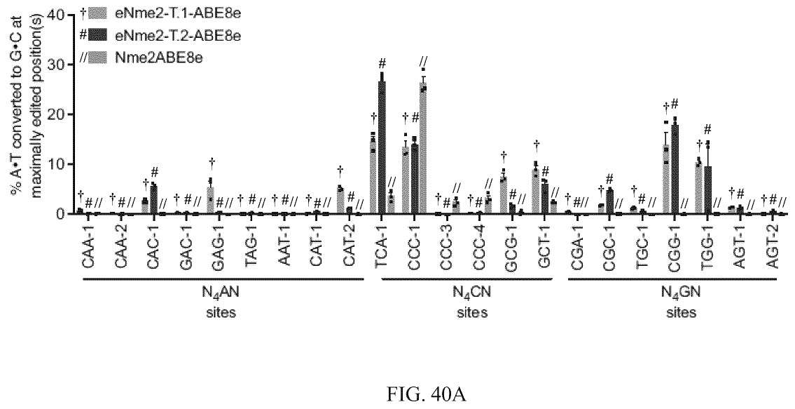

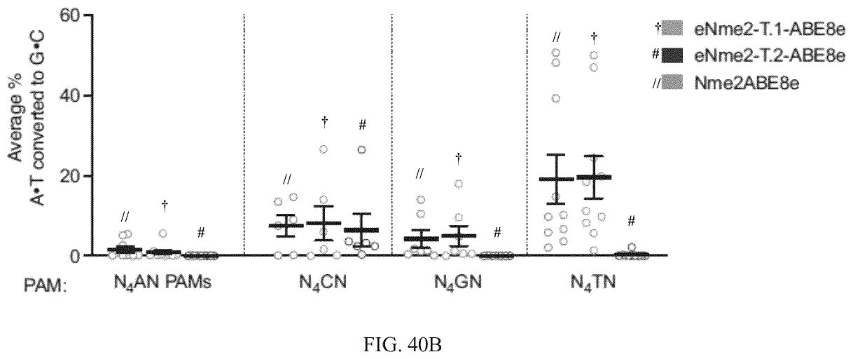

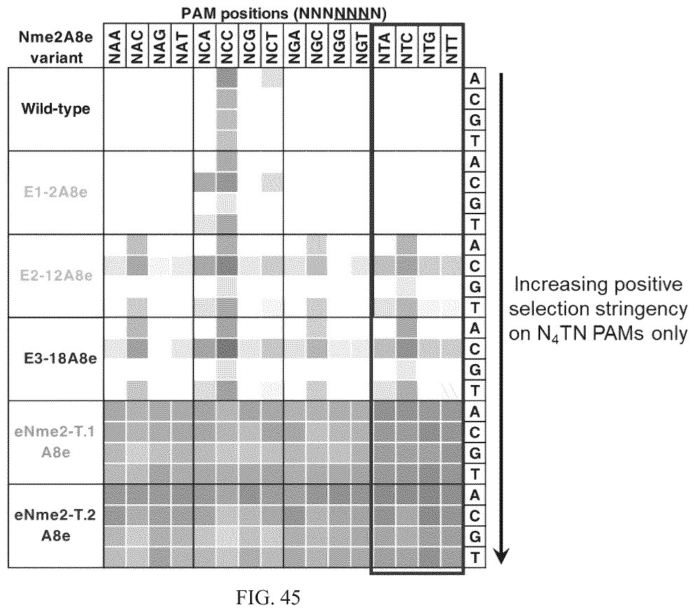

While the N₄TN activity of eNme2-T.1 and eNme2-T.2 (SEQ ID NO: 3) were promising, ABE-PPE data (FIG. 2C and FIG. 45) suggested that these two variants may also have activity on other PAM sites. To further characterize the PAM compatibility of these two variants in mammalian cells, eNme2-T.1-ABE8e and eNme2-T.2 (SEQ ID NO: 3) were evaluated at 22 genomic sites flanked by N₄VN PAMs (where V=A, C, or G). Consistent with their evolutionary histories and with ABE-PPE showing strongest enrichment for N₄TN, activity on N₄VN PAM sites was generally lower than on N₄TN PAM sites and varied considerably from site to site (FIG. 40A-40B). These mammalian cell editing data suggest that while eNme2-T.1-ABE8e and eNme2-T.1-ABE8e are capable of accessing N₄TN PAMs, and some other PAMs, editing efficiencies especially for the latter remain site-dependent. Together, these evolved variants from both trajectories (eNme2-C(SEQ ID NO: 1), eNme2-T.1 (SEQ ID NO: 2), and eNme2-T.2 (SEQ ID NO: 3)) offer access to a large suite of pyrimidine-rich PAMs largely inaccessible to SpCas9-derived variants while also representing the first reported evolution of a non-S. pyogenes Cas protein towards single-nucleotide PAM recognition.

Comparison of eNme2 and SpRY Base Editors and Nucleases

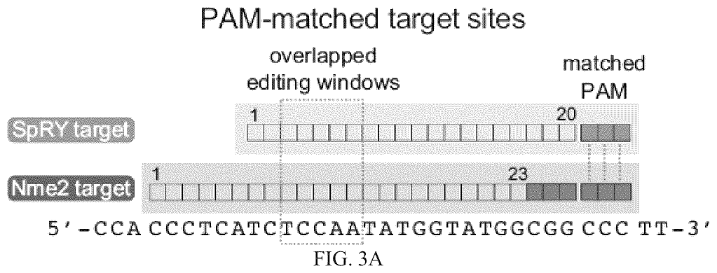

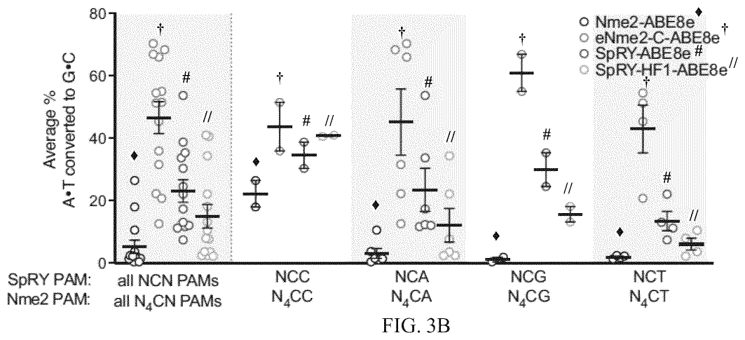

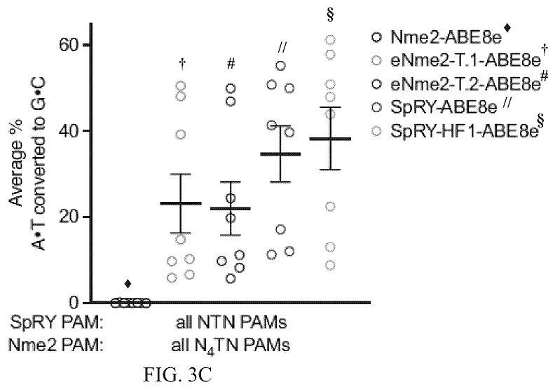

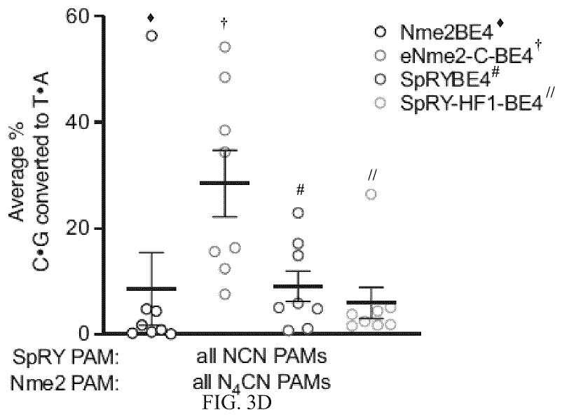

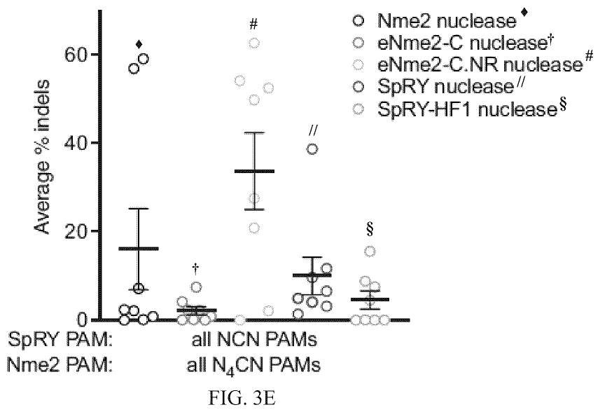

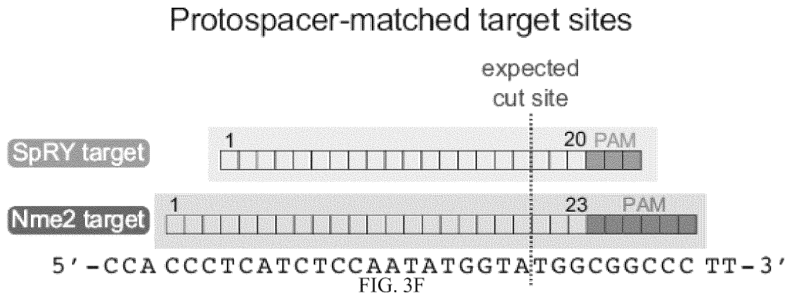

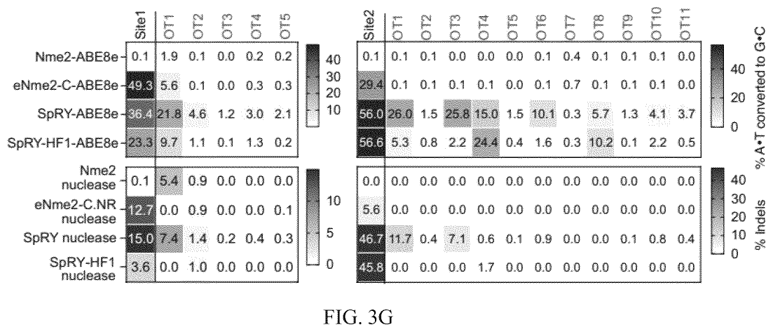

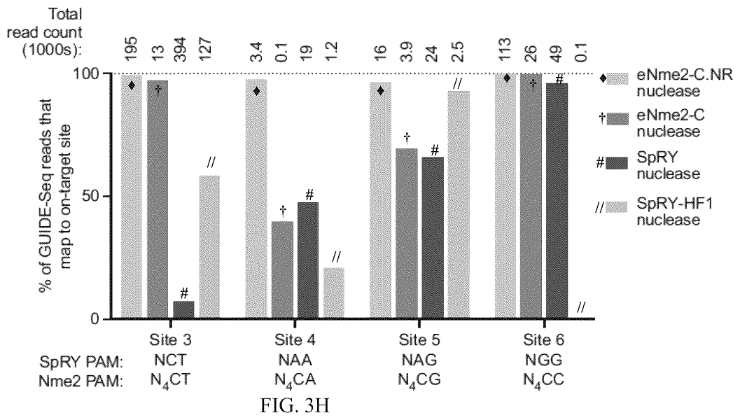

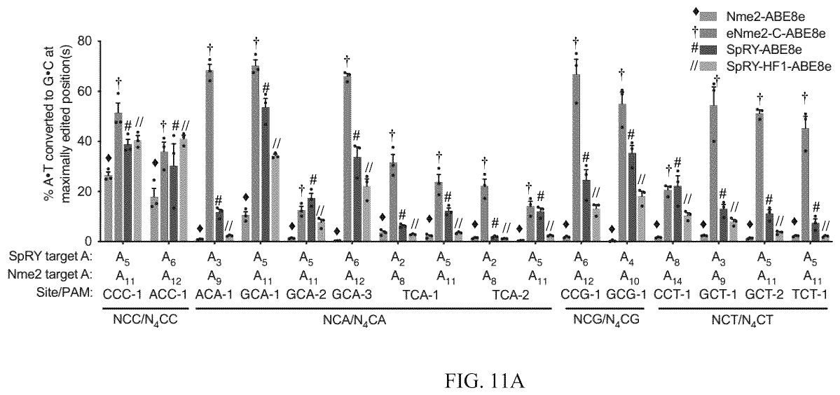

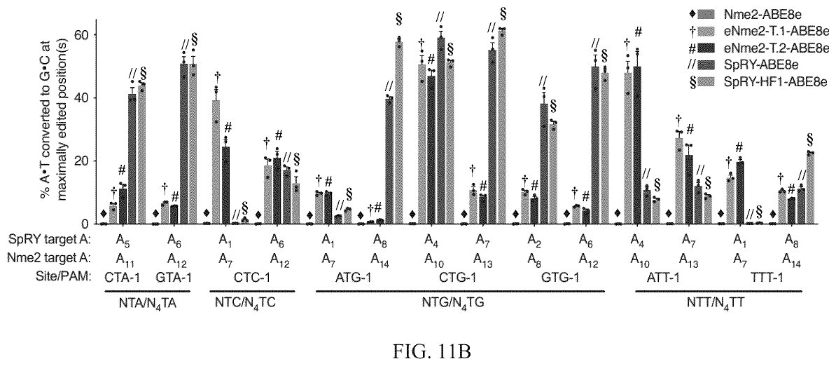

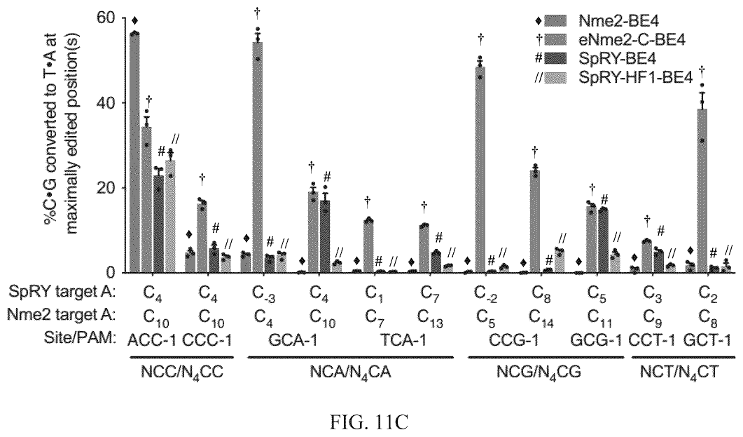

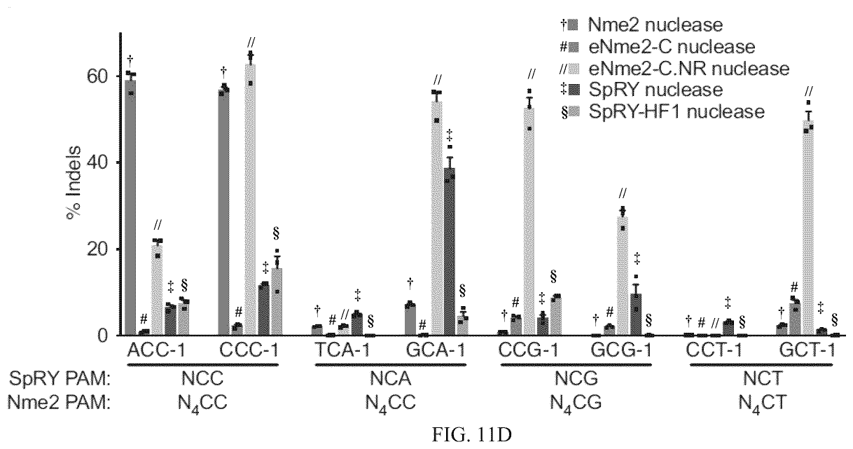

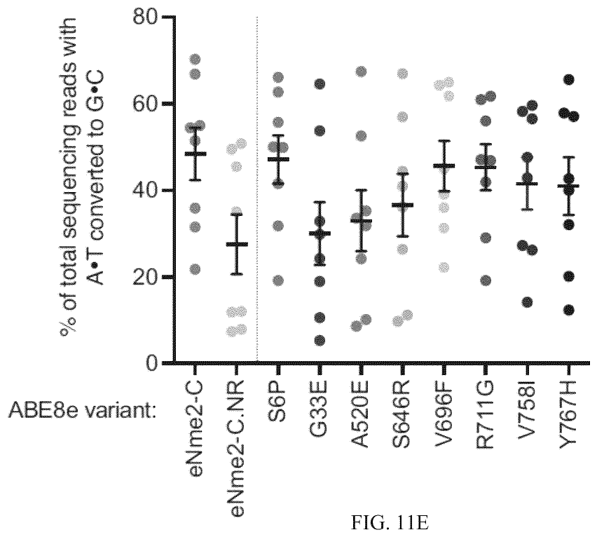

Next, the editing performance of evolved eNme2 variants was compared with that of alternative Cas variants. No natural Cas variants capable of targeting single pyrimidine PAMs have been reported⁸. Among engineered Cas variants, only SpRY has shown activity on some NCN and NTN PAMs¹⁴. PAM-matched genomic sites were selected to directly compare the base editing activities of SpRY and eNme2 variants (FIG. 3A). At 14 matched C-containing PAM sites in HEK293T cells, eNme2-C-ABE8e showed a marked improvement in adenine base editing over SpRY, averaging 47% vs. 23% A•T-to-G•C editing. This difference is more pronounced (47% vs. 15% A•T-to-G•C editing) when compared to the ABE8e version of high-fidelity SpRY, SpRY-HF1-ABE8e (FIGS. 3B and 11A). In contrast, at eight matched T-containing PAM sites in HEK293T cells, eNme2-T.1-ABE8e and eNme2-T.2-ABE8e are less active than either SpRY-ABE8e or SpRY-HF1-ABE8e (23% and 22% for eNme2-T.1-ABE8e and eNme2-T.2-ABE8e versus 35% and 38% for SpRY-ABE8e or SpRY-HF1-ABE8e, respectively) (FIGS. 3C and 11B). These data indicated that eNme2-C(SEQ ID NO: 1)offers a best-in-class option for modifying C-containing PAM sites, while eNme2-T.1 and eNme2-T.2 (SEQ ID NO: 3) provide new options for targeting some T-containing PAMs together with the existing SpRY variants.

Next, it was determined if the improvements to Nme2Cas9 were generalizable to other Cas9-dependent editing modalities. At six PAM-matched target sites in HEK293T cells, eNme2-C-BE4 exhibited an average of 28% C•G-to-T•A editing, a 3.2- and 4.8-fold improvement over SpRY-BE4 and SpRY—HF1-BE4, respectively (FIGS. 3D and 11C). Although less efficient than eNme2-C-ABE8e, eNme2-C-BE4 is capable of C•G-to-T•A editing at levels comparable to (within 2-fold of) those reported for SpCas9 or SpCas9-derived CBE variants at their canonical purine-containing PAMs^{11,13,14,45,46}.

Surprisingly, when the RuvC-inactivating mutation D16A²⁰ was reverted, eNme2-C (SEQ ID NO: 1) nuclease was inefficient at generating indels in mammalian cell culture, averaging only 2.1% indels at eight N₄CN PAM sites (FIGS. 3E and 11D). It was considered that this was possibly due to the large number of mutations in the RuvC and HNH domains of eNme2-C(SEQ 1D NO: 1), some of which could be nuclease-inactivating. Indeed, when all mutations in the nuclease and associated linker domains were reverted, the resulting variant, eNme2-C.NR (SEQ ID NO: 4) (eNme2-C S6P, G33E, A520E, S646R, V696F, R711G, V7581, Y767H) had restored nuclease activity while retaining novel N₄CN PAM activity (average 34% indels across the same eight sites). However, reversion of these mutations had a negative impact on ABE activity, with eNme2-C.NR-ABE8e exhibiting 1.8-fold reduced A•T-to-G•C conversion compared to eNme2-C-ABE8e (FIG. 11E). These results suggested that mutations in the RuvC/HNH domains were important for robust base editing of the eNme2-C(SEQ ID NO: 1)variant, but the same mutations, if present, were detrimental to the subsequent activation or catalytic activity of eNme2-C.NR (SEQ ID NO: 4) nuclease (FIGS. 11E-11F, Example 2).

Having established two distinct sub-variants of eNme2-C(SEQ ID NO: 1)for either base editing or DNA cleavage, the eNme2-C.NR (SEQ ID NO: 4) nuclease was compared to the SpRY and SpRY-HF1 nucleases. Surprisingly, both SpRY and SpRY-HF1 nucleases were relatively inefficient at the NCN PAM-matched sites tested, being significantly outperformed by eNme2-C.NR (SEQ ID NO: 4) nuclease (3.4- and 7.3-fold more efficient editing by eNme2-C.NR (SEQ ID NO: 4) nuclease, respectively) (FIGS. 3E and 11D). Given this data, perhaps some mutations in SpRY, like with eNme2-C(SEQ ID NO: 1), could asymmetrically affect base editing versus nuclease activities (for instance sufficient R-loop formation for base editing but slow conformational shift for nuclease activation^{47,48}). This would also potentially explain why the activity observed for SpRY-ABE8e appears to be much more generalizable at NYN PAMs than what would be expected given the limited NYN PAM scope initially described for SpRY nuclease¹⁴. Together, these data highlight eNme2-C(SEQ ID NO: 1)base editors and eNme2-C.NR (SEQ ID NO: 4) nucleases as highly effective variants for genome editing, offering promising alternatives to SpRY and SpRY-HF1 in applications requiring access to C-containing PAMs.

Off-Target Analysis Reveals High Genome-Wide Specificity of eNme2-C Variants

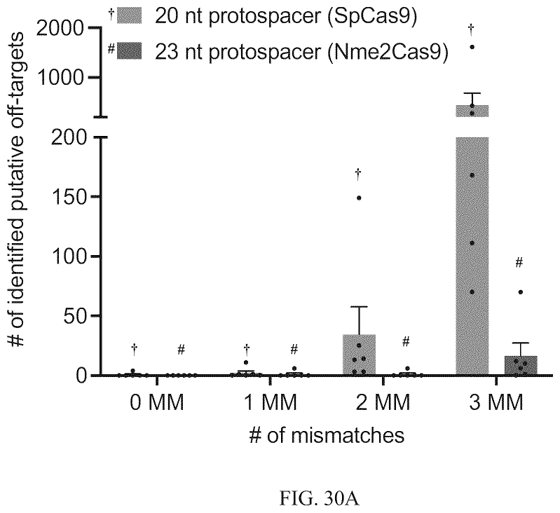

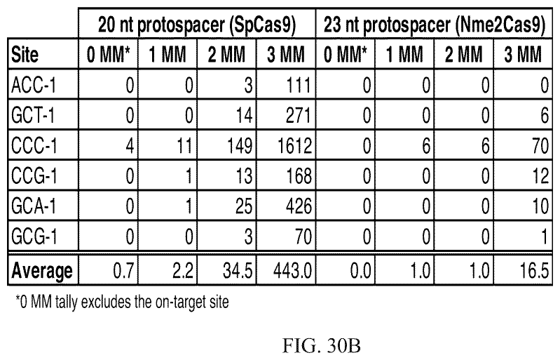

PAM-broadened Cas variants have been shown to increase off-target activity due to the increased number of sequences recognized as a PAM^{11,13,14}. While this off-target activity can be compensated for by introducing high-fidelity mutations that increase protospacer-target binding fidelity^{14,49}, these mutations can sometimes result in a reduction in overall Cas activity (FIGS. 3B, 3C, and 3E comparing SpRY to SpRY-HF1 variants). Nme2Cas9 has been shown to be highly accurate, exhibiting very few, if any, off-targets compared to SpCas9 at protospacer-matched sites²⁰. This higher specificity could potentially due to the longer protospacer requirement of Nme2Cas9 (22-23 nt²⁰ versus 20 nt), which naturally increased the total possible sequence space and decreased the occurrence of perfectly or near-perfectly matched off-target sites (FIG. 30A-30B). It was considered that the C-PAM specific eNme2-C (SEQ ID NO: 1) may also be more specific than PAM-broadened SpCas9 variants.

To evaluate off-target activity, two protospacer-matched sites (Site 1 and Site 2) were selected with validated nuclease and ABE activities for eNme2-C/eNme2-C.NR and SpRY variants (FIG. 3F). Using CHOPCHOPv3⁵⁰, in silico prediction was used to identify the set of potential off-target sites with ≤2 mismatches and no more than one PAM proximal (within 10 bp of the PAM) mismatch to at least one of the two protospacers (23 nt for Nme2Cas9, 20 nt for SpRY). The off-target nuclease and ABE8e activities at all identified off-target sites (seven for Site 1, twelve for Site 2) were evaluated using targeted amplicon sequencing.

For the Site 1 protospacer, live of the seven predicted sites sequenced well, and eNme2-C-ABE8e showed off-target base editing >1% at one of these five sequenced off-target sites, while eNme2-C.NR (SEQ ID NO: 4) did not generate any off-target indels >1% (FIG. 3G). In contrast, SpRY-ABE8e and SpRY-HF1-ABE8e exhibited off-target base editing >1% at all five or four of five sites, respectively, despite having lower on-target efficiency than eNme2-C-ABE8e. As nucleases, SpRY and SpRY-HF1 showed higher fidelity, with only two of five or one of five off-target site(s) exhibiting indels >1%, respectively. Similar trends were observed for the Site 2 protospacer. No off-target base editing or indel formation >1% was observed at any of the twelve sequenced off-target sites for eNme2-C-ABE8e or eNme2-C.NR (SEQ ID NO: 4), whereas off-target base editing and indel formation >1% was observed at many sites for SpRY and SpRY-HF1. These data suggested that eNme2-C-ABE8e and eNme2-C.NR (SEQ ID NO: 4) retain the high natural specificity of Nme2Cas9 and offer greater specificity than their SpRY and SpRY-HF1 counterparts, particularly for precision applications such as base editing.

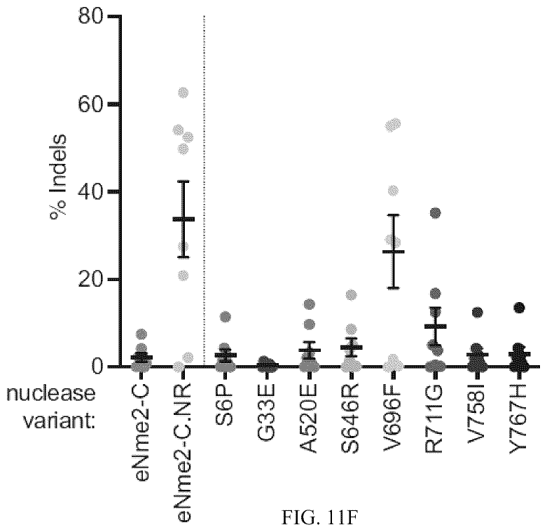

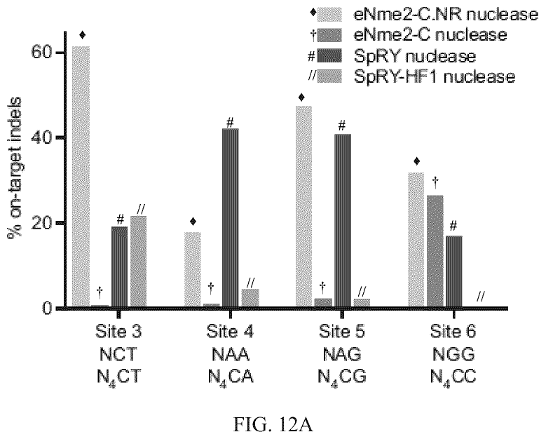

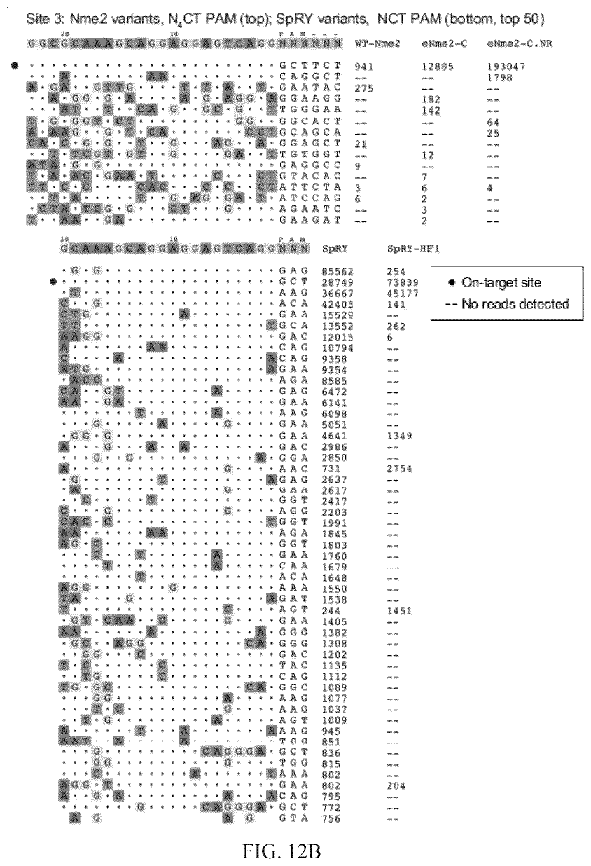

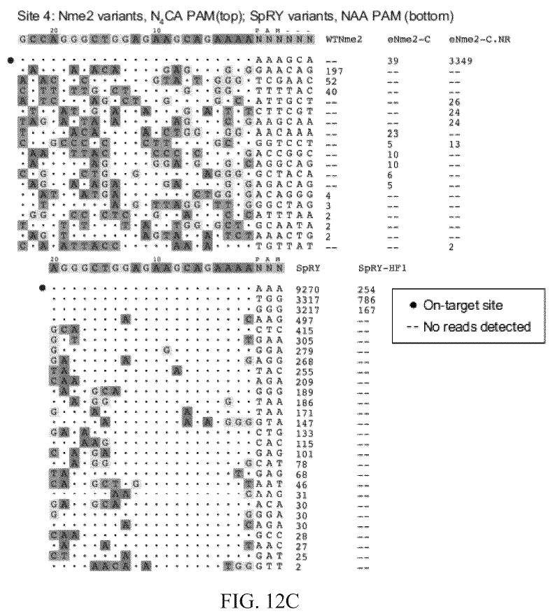

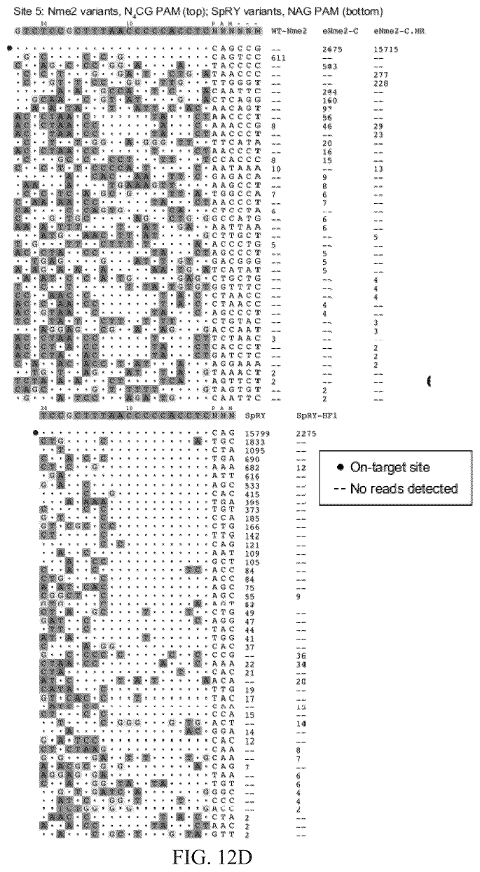

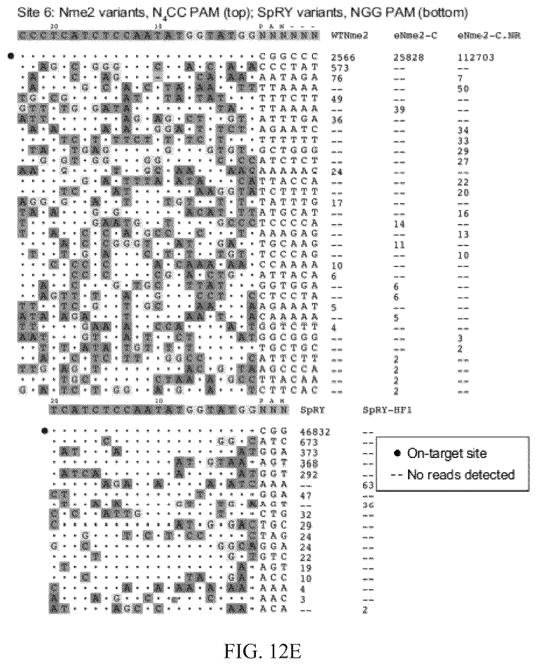

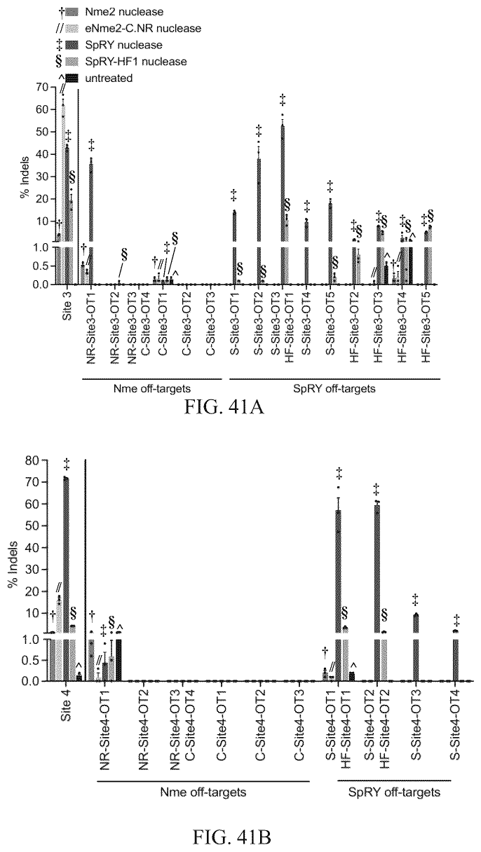

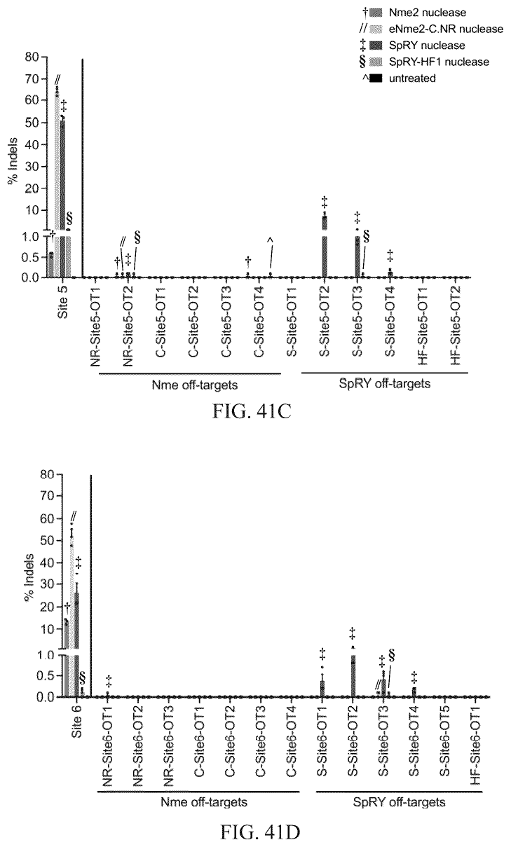

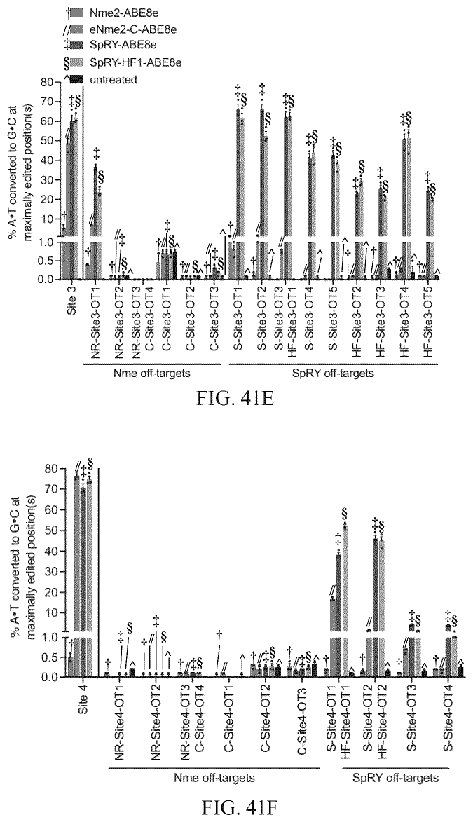

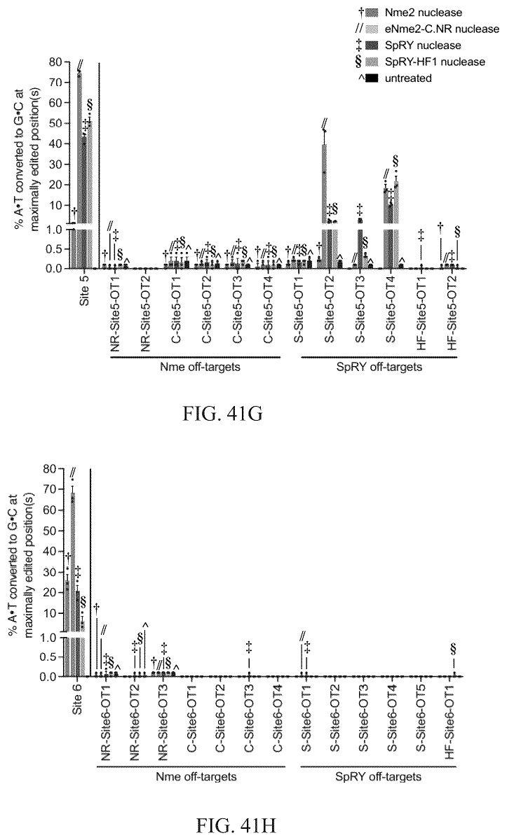

To perform a more unbiased, genome-wide survey of potential off-targets, GUIDE-seq⁵¹ was used to evaluate double-strand breaks generated by eNme2-C.NR (SEQ ID NO: 4) compared to SpRY variants at four protospacer-matched sites. Targeted sequencing of the on-target sites in treated U2OS cells showed robust indel formation at all four sites for both SpRY nuclease and eNme2-C.NR (SEQ ID NO: 4) (30% and 40% indels for SpRY nuclease and eNme2-C.NR nuclease, respectively). Despite 3 of the 4 sites containing NRN-PAMs, SpRY-HF1 nuclease generated >10% indels at the fourth site containing an NCN PAM. The nuclease-active version of eNme2-C(SEQ ID NO: 1) was also included, although indel formation was inefficient (<10%) at all but one site (FIG. 12A). Across all four sites, eNme2-C.NR (SEQ ID NO: 4) exhibited high specificity, averaging 52-to-1 on-to-off-target reads, compared to SpRY which averaged a 1.2-to-1 on-to-off-target ratio (FIGS. 3H and 12B-12E). These specificity values corresponded to a range of 7 to 22 putative off-target sites for eNme2-C.NR (SEQ ID NO: 4) versus 14 to 591 putative off-target sites for SpRY. At the site on which it was active, eNme2-C(SEQ ID NO: 1) similarly exhibited minimal off-target activity. In contrast, while SpRY-HF1 exhibited higher specificity than SpRY at the site on which it was active (Site 3), it still induced substantial off-target editing compared to eNme2-C.NR (SEQ ID NO: 4) (FIG. 3I). The top GUIDE-Seq-nominated loci for eNme2-C(SEQ ID NO: 1), eNme2-C.NR (SEQ ID NO: 4), SpRY, and SpRY-HF1 were sequenced. In agreement with the GUIDE-Seq data, both SpRY and SpRY-HF1 exhibited off-target nuclease and adenine base editing at more sites than either eNme2-C.NR (SEQ ID NO: 4) nuclease or eNme2-C-ABE8e (FIG. 41A-41H).

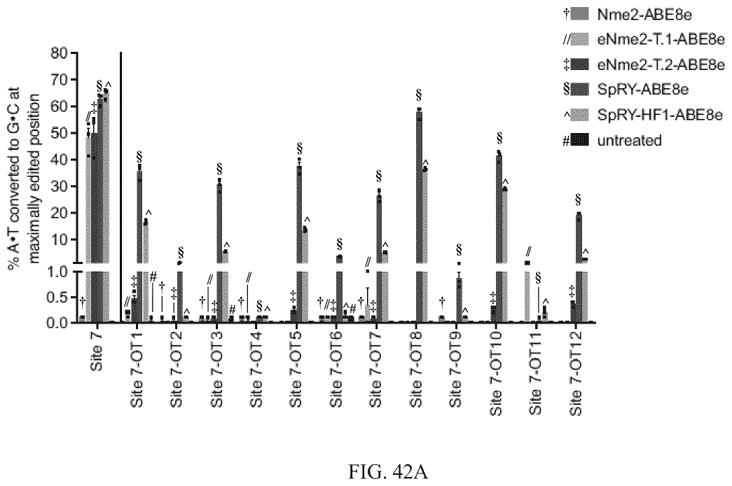

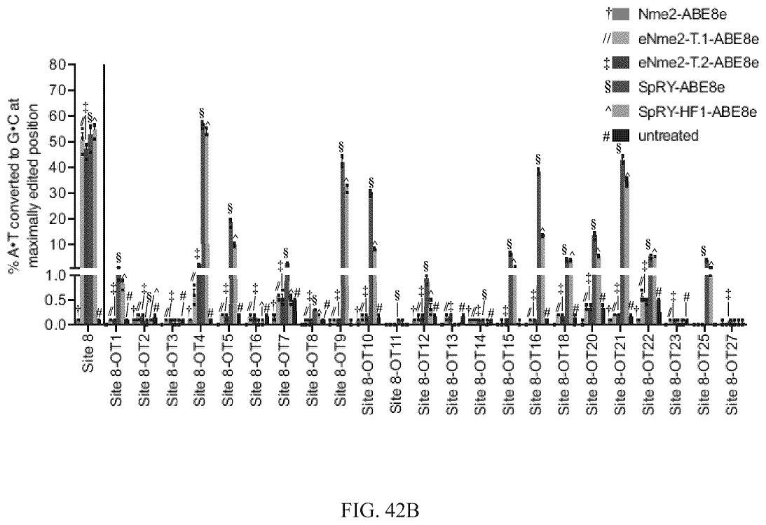

Similarly, sequencing was performed at in silico-nominated off-target sites for protospacer-matched sites comparing eNme2-T.1-ABE8e and eNme2-T.2-ABE8e to SpRY-ABE8e and SpRY-HF1-ABE8e. As with eNme-C, both eNme2-T variants exhibited off-target base editing at fewer sites than either SpRY or SpRY-HF1 (FIGS. 42A and 42B). Together, these results indicate that evolved Nme2Cas9 variants may offer improved specificity compared to SpRY variants.

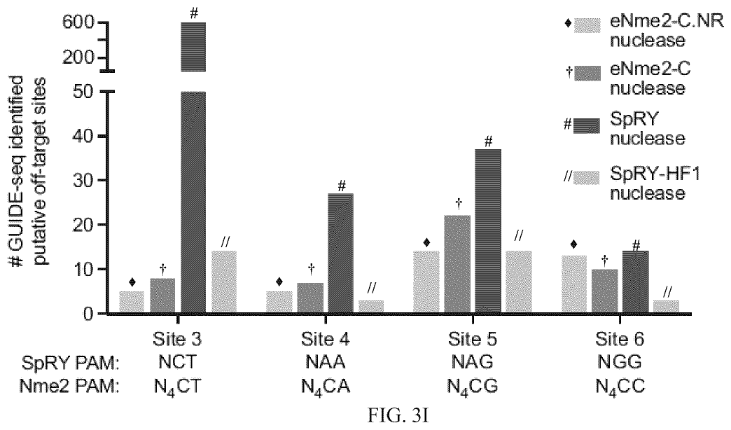

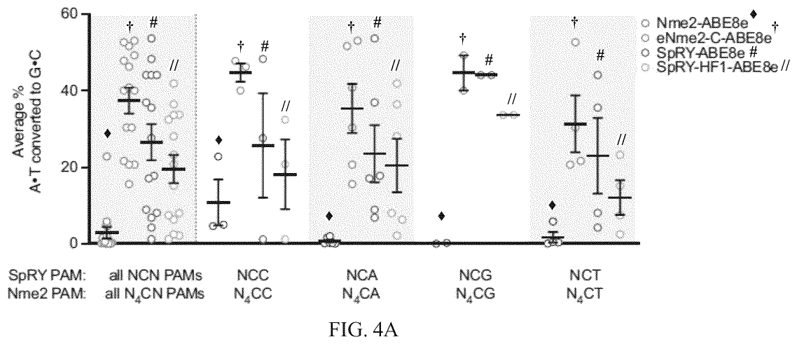

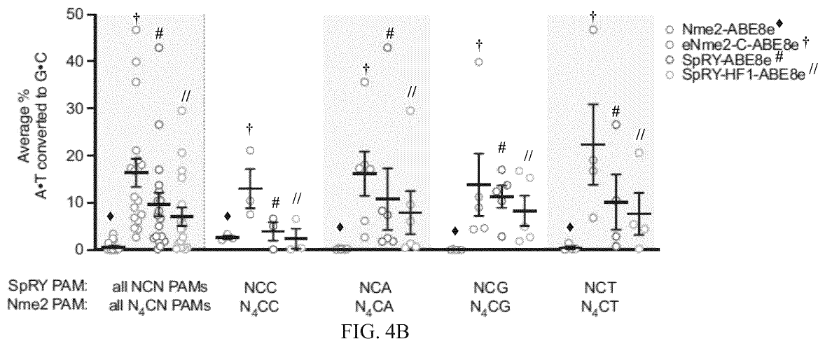

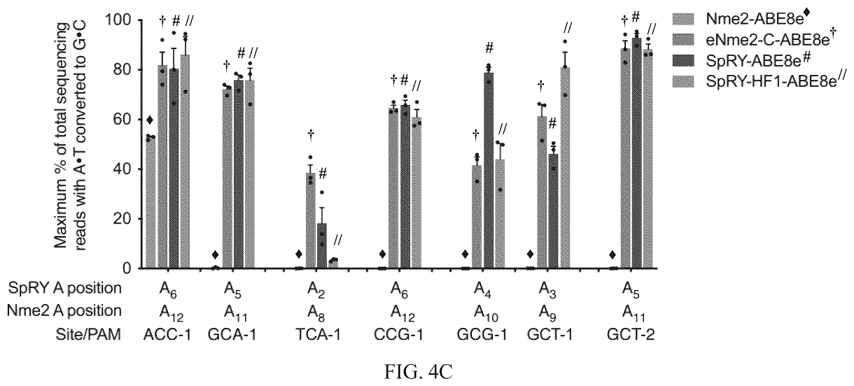

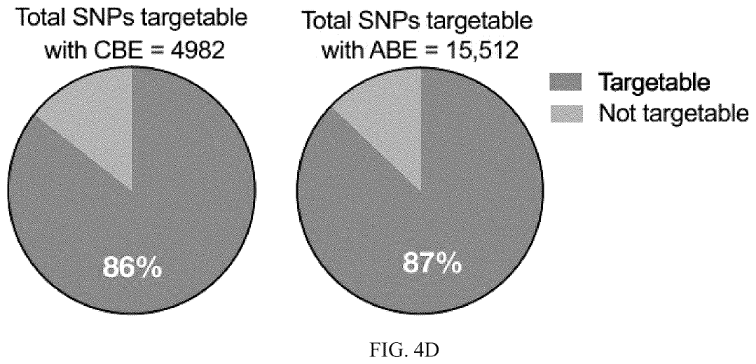

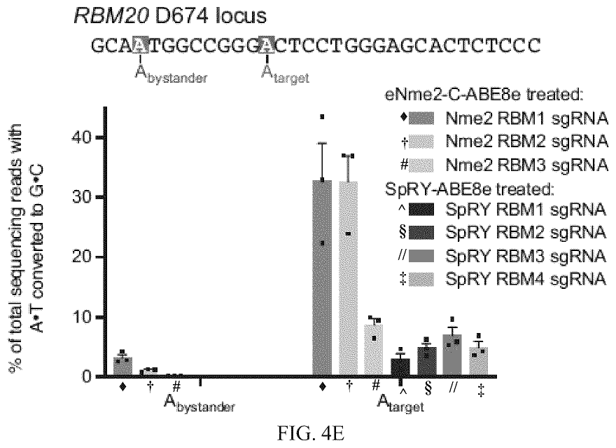

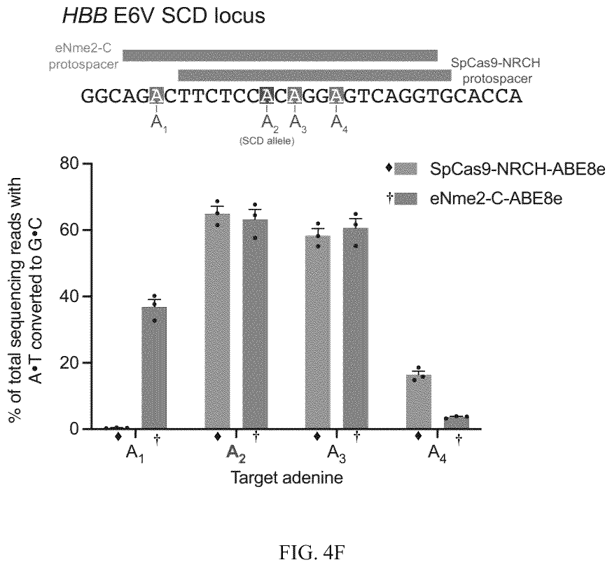

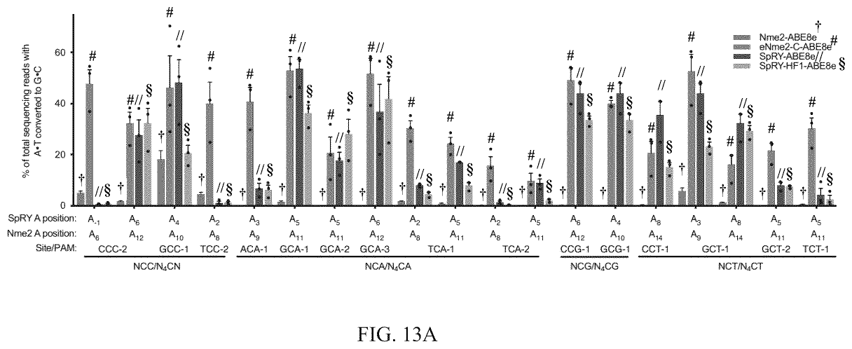

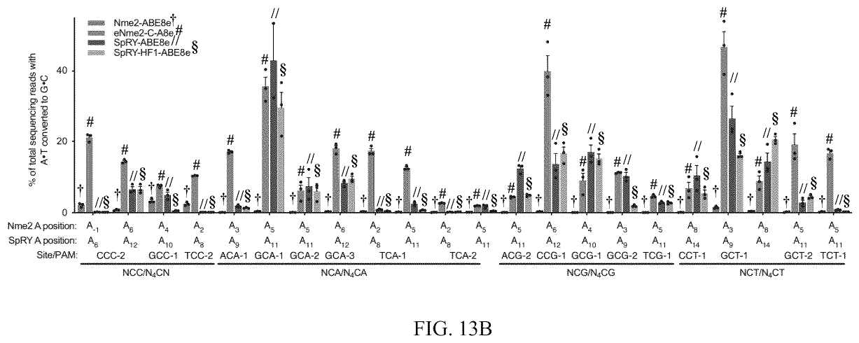

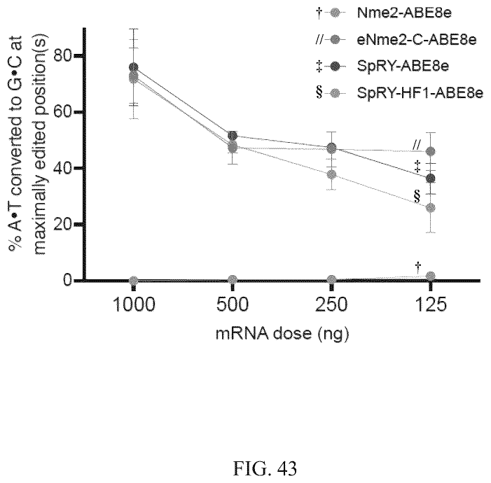

eNme2-C(SEQ ID NO: 1) is active in multiple mammalian cell types and enables access to both existing and new target SNPs Having validated the high-efficacy and specificity of eNme2-C(SEQ ID NO: 1) at target sites containing N₄CN PAMs, generalizability was demonstrated in multiple cell types. In an immortalized hepatocyte cell line, HUH7, eNme2-C-ABE8e retained its broad base editing activity across sites containing N₄CN PAMs, accessing all 15 sites tested with an average of 37% A•T-to-G•C base editing (FIGS. 4A and 13A). Similarly, at 18 sites in U2OS cells, adenine base editing activity was seen at all sites, albeit at lower average efficiency (averaging 16% A•T-to-G•C editing) (FIGS. 4B and 13B). In both cell types, eNme2-C-ABE8e outperformed SpRY-ABE8e and SpRY-HF1-ABE8e, although the extent varied. Finally, primary human dermal fibroblasts were nucleofected with eNme2-C-ABE8e mRNA, achieving 64% A•T-to-G•C base editing across seven endogenous sites (FIG. 4C). Notably, eNme2-C-ABE8e, SpRY-ABE8e, and SpRY-HF1-ABE8e performed equally well in this cell line with nucleofection, potentially due to the high efficacy of mRNA nucleofection (FIG. 43)⁹¹¹. Together, these data demonstrated that eNme2-C(SEQ ID NO: 1) was a broadly applicable Cas protein enabling precision genome editing in multiple biologically relevant cell types.

Because of its N₄CN PAM activity, eNme2-C(SEQ ID NO: 1) was complementary to single-G recognizing SpCas9 variants SpCas9-NG¹³ and SpG¹⁴, which were estimated to enable potential cleavage every ˜2.2 bp in the human coding sequence¹³. As a cytosine or adenine base editor, eNme2-C(SEQ ID NO: 1) enabled access to 86% and 87% of pathogenic transition SNPs, respectively, recognized in the ClinVar database (FIG. 4D)^{52,53}. Although SpRY base editors should access similar PAMs due to its near-PAMless nature, it was determined that differences in editing windows and specific PAM compatibilities would enable eNme2-C(SEQ ID NO: 1) base editors to not only serve as higher-fidelity alternatives to SpRY base editors, but also facilitate access to new targets.

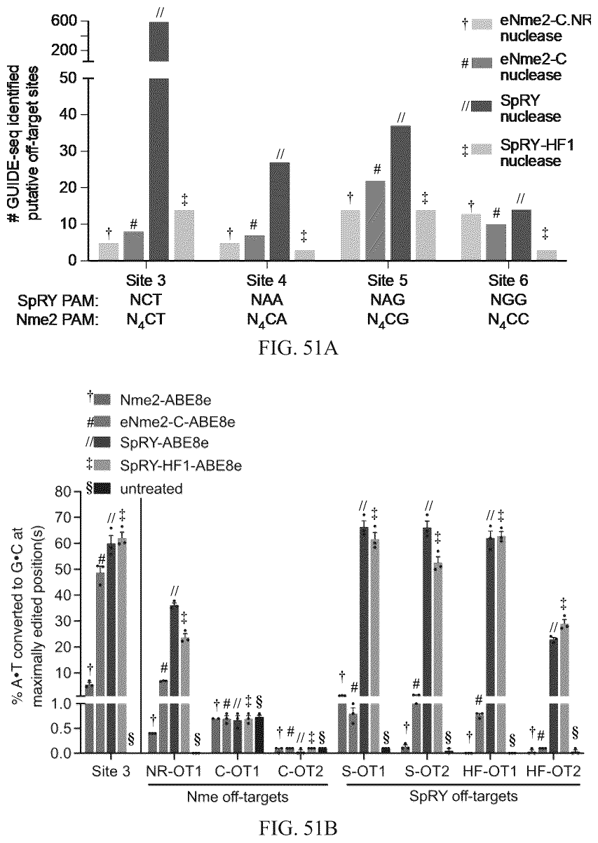

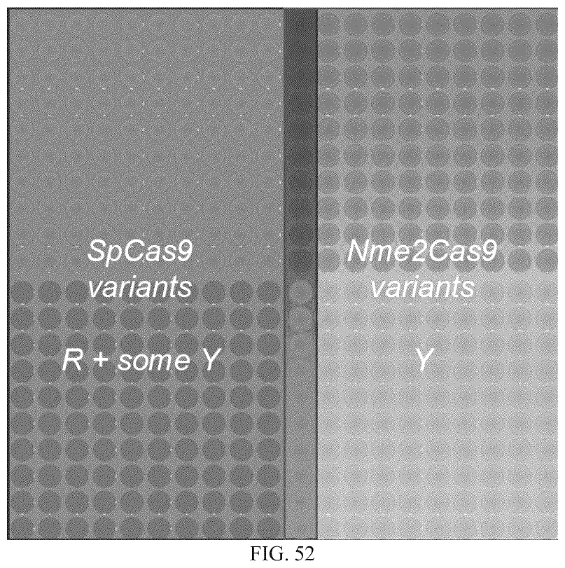

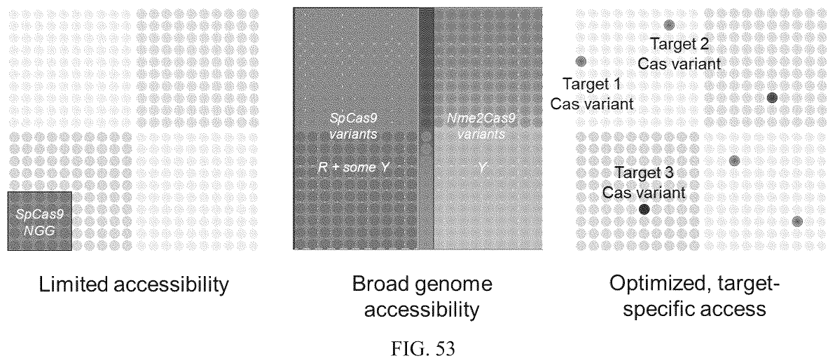

RBM20 is a gene encoding a trans-activating splicing factor, and mutations in the gene have been observed in 2-3% of familial dilated cardiomyopathy cases⁵⁴. While many mutations have been identified in the coding sequence of RBM20, the individual effect of these mutations have not been well characterized, potentially due to the difficulty of installing some of these mutations in isolation. eNme2-C-ABE8e was used to install the D674G mutation, an A•T-to-G•C transition in which the target base was upstream of a stretch of pyrimidine bases inaccessible to most characterized Cas variants. All three eNme2-C-ABE8e guides tested enabled editing of the target adenine, with the optimal guide reaching 33% A•T-to-G•C base editing. In contrast, none of the four SpRY guides placing the target adenine in the optimal editing window of SpRY (positions 4-7)⁹ were able to achieve >10% A•T-to-G•C conversion (FIG. 4E). This data demonstrated that eNme2-C(SEQ ID NO: 1) enables the study and potential correction of previously inaccessible pathogenic SNPs (FIG. 51A-53).

Finally, it was examined whether eNme2-C-ABE8e could edit previously targeted therapeutically relevant loci with reduced off-target editing frequencies. Adenine base editing of the sickle-cell allele (HBB^{S}) results in the benign hemoglobin Makassar allele and can rescue sickle cell disease in animals^{1,55}. While a previously evolved SpCas9 variant, SpCas9-NRCH, can efficiently install the Makassar allele in both cell culture and mouse models^{1,11}, off-target base editing was observed at several Cas-dependent off-target sites.

It was tested if the highly specific nature of eNme2-C(SEQ ID NO: 1) may yield a more favorable off-target editing profile at this locus. In a HEK293T cell line containing the SCD E6V mutation⁵⁶, an eNme2-C-ABE8e sgRNA resulted in comparable editing efficiency of the target adenine compared to optimized SpCas9-NRCH-ABE8e and sgRNA (63% versus 65% A•T-to-G•C conversion, respectively) (FIG. 4P). Due to the slightly shifted editing window of eNme2-C-ABE8e, it was observed that a higherediting at an upstream bystander adenine, and lower editing at a downstream bystander adenine. With respect to off-target activity, it was observed that much higher specificity for eNme2-C-ABE8e compared to SpCas9-NRCH-ABE8e when targeting this site. Using the same in silico criteria described above, nine predicted off-target sites for eNme2-C(SEQ ID NO: 1), and 11 predicted off-target

| Target site | | | SEQ | | Mismatches | |
|---|---|---|---|---|---|---|
| or off- | | Protospacer sequence | ID | | (PAM | Previously |
| target site | Gene | (5′ to 3′) | NO | PAM | proximal\hat{ }) | validated? |
| HBB-eNme2-C | HBB | AGACTTCTCCACAGGAGTCAGGT | 121 | GCACCA | — | |
| HBB-Nme-OT1 | HBB | AGACTTCTCCtCAGGAGTCAGaT | 124 | GCACCA | 2(1) | Overlaps HBB- |
| | | | | | | NRCH-OT2 |
| HBB-Nme-OT2 | Intergenic | ttACTTCTCCACAGcAGTCAGGT | 125 | TTTGAT | 3(1) | |
| HBB-Nme-OT3 | CNTN5 | AaACTTCTCCACtGGAGTCAGGg | 126 | CAGGAG | 3(1) | |
| HBB-Nme-OT4 | Intergenic | gagCTTCTtCACAaGAGTCAGGT | 127 | AACCTA | 5(1) | |
| HBB-Nme-OT5 | CGNL1 | ccctTTCTCCACAGGAGTCAGGa | 128 | GAGACA | 5(1) | Overlaps HBB- |
| | | | | | | NRCH-OT3 |
| HBB-Nme-OT6 | NUP50 | tcACTTCTCCAGAGGTGTCAGGT | 129 | AACTAA | 3(1) | |
| HBB-Nme-OT7 | Intergenic | ctgCTTCTCCAcAaGAGTCAGGT | 130 | ATCACT | 5(1) | |
| HBB-Nme-OT8 | Intergenic | AagtTTCTCCAGAGGAGTCAGGT | 131 | TAGGAG | 3(0) | Overlaps HBB- |
| | | | | | | NRCH-OT10 |
| HBB-Nme-OT9 | Intergenic | cttCTTCTCCAtAGGAGTCAGaT | 132 | GTGATG | 5(1) | Overlaps HBB- |
| | | | | | | NRCH-OT11 |
| HBB-SpCas9- | HBB | TTCTCCACAGGAGTCAGGTG | 133 | CAC | — | |
| NACH | | | | | | |
| HBB-NRCH-OT1 | TET1 | TaaTCCACAGGAGTCAGGTG | 136 | CAC | 2(0) | Y⁴ |
| HBB-NRCH-OT2 | HBB | TTCTCCTCAGGAGTCAGaTG | 137 | AGA | 2(1) | Y⁴ |
| HBB-NRCH-OT3 | CGNL1 | TTCTCCACAGGAGTCAGGaG | 188 | ATA | 1(1) | |
| HBB-NRCH-OT4 | PCSK6 | TTCTCCAgAGGAGTCAGGgG | 189 | GGT | 2(1) | Y⁴ |
| HBB-NRCH-OT5 | CFDP1 | cTCcCCACAGGAGTCAGGTG | 140 | CCT | 2(0) | Y⁴ |
| HBB-NRCH-OT6 | ETFB | TTCTCCtCAGGAGTCAGGaG | 141 | GGC | 2(1) | Y⁴ |
| HBB-NRCH-OT7 | Intergenic | TTCTCCcCAGGAGCCAGGTG | 172 | GCC | 2(1) | Y⁴ |
| HBB-NRCH-OT8 | LOC1053 | TTCTgCACAGGAGTCAtGTG | 173 | AAG | 2(1) | Y⁴ |
| | 77396 | | | | | |
| HBB-NRCH-OT9 | LOC1053 | TTCTCCcCgGGAGTCAGGTG | 174 | CAC | 2(0) | Y⁴ |
| | 74898 | | | | | |
| HBB-NRCH-OT10 | Intergenic | TTCTCCAgAGGAGTCAGGTt | 175 | AGG | 2(1) | |
| HBB-NRCH-OT11 | Intergenic | TTCTCCATAGGAGTCAGaTG | 176 | TGA | 2(1) | Y⁴ |

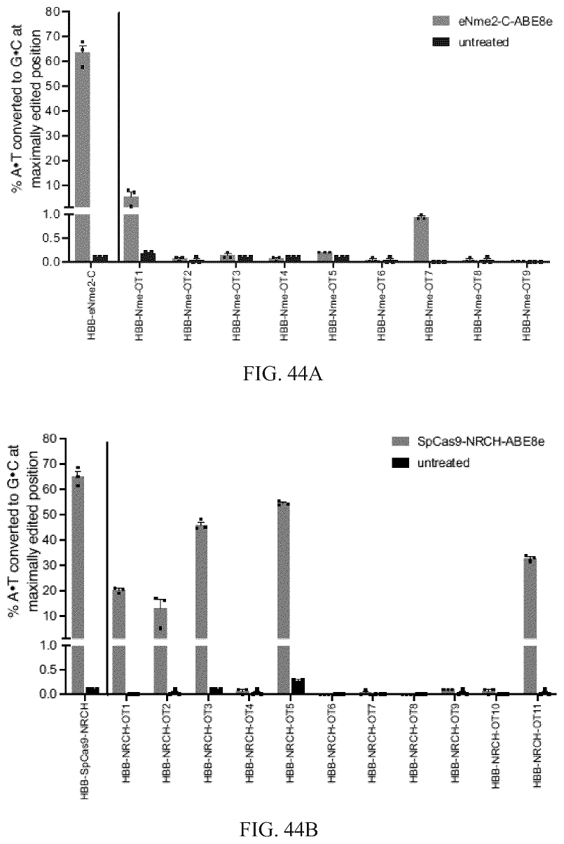

sites for SpCas9-NRCH (Table 4) were selected. Across the nine predicted off-target sites for eNme2-C-ABE8e, off-target base editing was
observed at only two of the nine predicted sites, and neither exceeded 10% A•T-to-G•C conversion. In contrast, off-target editing with SpCas9-NRCH-ABE8e was observed at five of 11 predicted sites and averaged 33% A•T-to-G•C conversion across those five sites (FIG. 44A-44B). Together, these data further support that eNme2-C(SEQ ID NO: 1) not only expands the targeting scope of base editors but may also offer a more site-specific alternative to existing Cas9 variants.
   Table 4: CHOPCHOPv3-Identified Off-Target Sites of sgRNAs Targeting the HBB Sickle-Cell Disease Mutation

### Example 2

### Development of a Phage-Assisted Continuous Negative Selection for Cas Proteins

Phage assisted continuous evolution (PACE) is a valuable tool for tailoring the activity of desired proteins of interest (POIs), because of its rapid rate of diversification and selection relative to stepwise or library-based protein evolution methods. In the context of large proteins with multiple complex activities like Cas enzymes, PACE is particularly powerful due to its ability to quickly and agnostically explore sequence space throughout the POI. This broad sequence exploration is in stark contrast to library-based rational engineering methods, which typically focus on small, promising regions of a given POT due to library-size and cost constraints. Given the prior success of PACE in generating highly mutated variants of SpCas9 and Nme2Cas9 with increased PAM compatibility, it was considered that a dual positive/negative PACE selection could be used to not broaden but shift PAM compatibility. Illustrations of exemplary selection schemes and PAM promiscuity is shown in FIG. 31. Briefly, among selection schemes that only require evolving Cas variants to acquire novel PAM compatibility, a common outcome is PAM promiscuity, in which activity on new PAMs is observed while activity on wild-type or original PAMs is retained. This promiscuity can be reduced via introduction of a counterselection on the undesired, original PAM(s) while continuing to select for increased activity on novel, target PAMs. This type of dual positive/negative selection thereby enables the evolution of more PAM specific variants that may have more desirable onto off-target activity profiles due to limited PAM promiscuity (FIG. 31). By doing so, increased on-target activity at desired PAMs could be decoupled from the increased off-target activity that typically accompanies PAM-broadened variants.

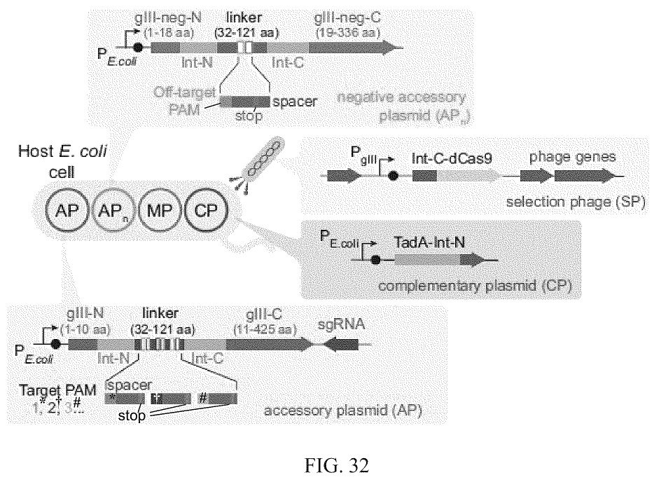

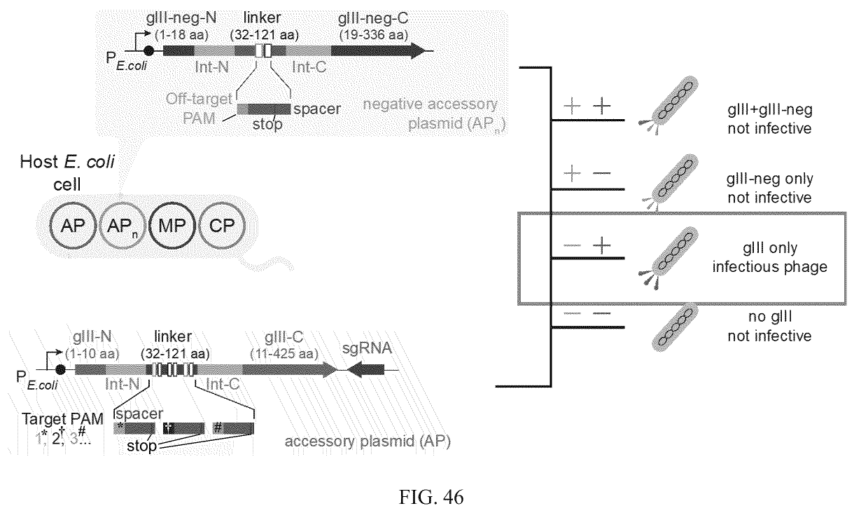

To develop a generalizable dual positive/negative selection for Cas protein PAM variants, a negative selection within the prior SAC-PACE framework was introduced (FIG. 46-48). In SAC-PACE, the essential gene necessary for M13 bacteriophage propagation, gIII, was placed on an accessory plasmid (AP) and split by an in cis intein in which the N- and C-terminal intein halves were fused together with an arbitrary linker (31-121 aa) comprised of one or more target protospacer/PAM combinations that contain at least one stop codon each. This linker may be reprogrammed with any sequence context and contains one or more stop codons which prevent the expression of gIII. On the selection phage (SP), a Cas protein was expressed fused to either an adenosine deaminase or an intein that may undergo in trans splicing with an adenosine deaminase-intein fusion expressed from a complementary plasmid (CP) in host cells. Successful Cas engagement with a programmed protospacer/PAM containing the stop codons within the linker results in subsequent base editing that enabled phage propagation. To enable negative selection, a new negative accessory plasmid (APₙ), which has similar construction as the AP, except with gIII-neg, a dominant negative form of gIII that prevents phage propagation was introduced, instead of gIII (FIG. 32). The construct expressing gIII-neg was split by a fused intein pair orthogonal to the one used in the AP, and the linker contained a stop codon flanked by an arbitrary, undesired PAM. Like the original SAC-PACE selection, the dual positive/negative SAC-PACE selection required functional Cas activity, including R-loop formation and maintenance which enabled subsequent base editing, while retaining high sequence generalizability such that any desired Cas protein may be evolved with this approach.

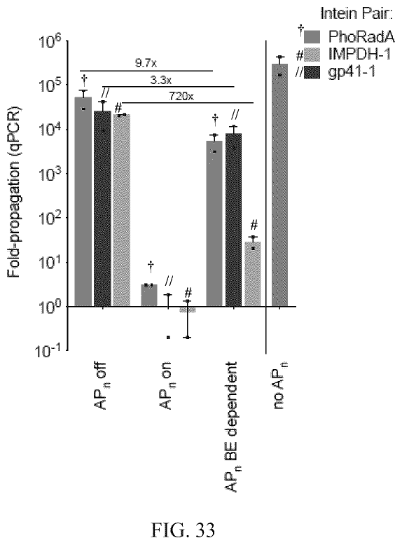

To validate the APₙ, a new orthogonal intein pair was identified that was not already in use in the complementary plasmid (CP, gp41-8 intein) used for expressing the adenosine deaminase TadA8e, or the AP (Npu intein). Next, selection of three split intein pairs previously shown to be orthogonal to both the Npu and gp41-8 intein pairs. Two test APₐs for each intein pair were constructed to test the maximum theoretical dynamic range of phage propagation for each pair. One APₙ (positive control) contains gill-neg with the intein pair fused in cis by an arbitrary linker inserted after Ser18 and conditionally expressed by a phage shock promoter (psp). The other APₙ, (negative control) contains the same construction except with one stop codon inserted within the arbitrary linker. These APₙs were tested in host cells containing split SAC-PACE components: an AP containing one PAM/protospacer combination with two stop codons, and a CP expressing a TadA8e R26G variant previously validated for split SAC-PACE. Notably, all three intein pairs exhibited a robust on-to-off dynamic range (>10-fold) with a minimal reduction in on-target propagation, suggesting that the APₙ architecture is compatible for activating gIII-neg expression without drastically affecting the positive selection (FIG. 33).

Next, to confirm that APₙ activation may be made dependent on undesired PAM activity. The same protospacer used in the AP was placed within the APₙ linker, flanked by a NNNNCC PAM that should be readily accessible to both wild-type Nme2Cas9 and evolved eNme2-C(SEQ ID NO: 1). Next, infected host cells containing split SAC-PACE components and this AP, with phage containing eNme2-C(SEQ ID NO: 1) in an overnight propagation assay. Overnight propagation assay was performed to test the ability of three different intein pairs to splice and enable functional gIII-neg expression from the APₙ in a base editing-dependent or independent manner. All host cells were infected with wild-type, Nme2Cas9-containing phage. The APₙ off condition was an APₙ with a stop codon flanked by a PAM that cannot be targeted by wild-type Nme2Cas9. The APₙ on condition was an APₙ without a Stop codon in the linker. The APₙ BE dependent condition was an APₙ with a stop codon flanked by a PAM that can be targeted by wild-type Nme2Cas9. The no APₙ condition does not contain an APₙ. Surprisingly, only the IMPDH-1 intein pair retained a high base editing dependent dynamic range (722-fold difference between off-and-on states) (FIG. 33). Both the PhoRadA and gp41-1 intein pairs had a <10-fold off-to-on ratio, perhaps suggesting that these inteins are less effective at generating competently spliced gIII-neg at lower expressed concentrations. Moving forward, the IMPDH-1 intein pair was used in APₙ constructions.

### Preliminary Evolution Campaigns Towards N₄TTN PAM-Specific Nme2Cas9 Variants

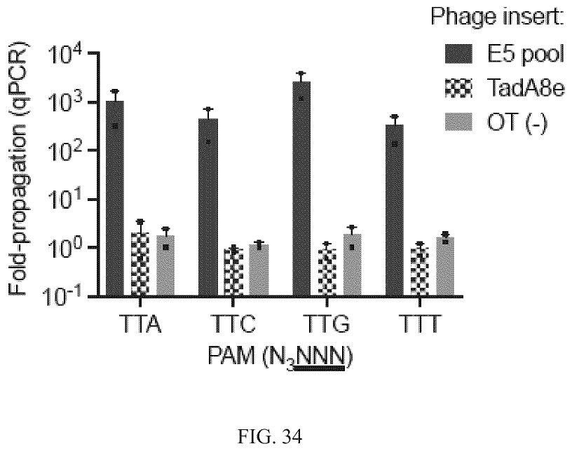

Having validated a BE-dependent dual positive/negative SAC-PACE selection, next it was desired to select desirable PAMs to target during evolution. In the previous campaigns to evolve Nme2Cas9, one of the primary limitations of the positive-only SAC-PACE selection was the acquisition of broad, promiscuous PAM activity that translated to inconsistent activity in mammalian cells. This outcome was particularly evident in the N₄TN-PAM trajectory that required more drastic changes in PAM recognition for Nme2Cas9, which canonically interacts with N₄CC PAMs. It was considered that more PAM-specific variants would not only enable greater specificity, but also higher on-target activity as the variants are no longer capable of being sequestered at off-target PAM sites. Four evolution campaigns were designed towards each of the four N₃TTN PAMs, with an initial APₙ penalizing activity on an N₃CCC PAM, a PAM accessible to wild-type Nme2Cas9 and still targeted in bacteria by variants previously evolved on N₄TN PAMs. For the positive selection, the selection stringency was further increased by including a third PAM (novel nucleobase identity at PAM positions 1-3 and 7) in addition to the two previously present in the dual PAM, split SAC-PACE strategy. Notably, Nme2Cas9 variants previously evolved on the dual PAM, split SAC-PACE selection (E5 phage) were still capable of propagating on these new triple PAM, split SAC-PACE APs at levels sufficient for PACE (FIG. 34).

To explore multiple PAMs and negative selection stringencies in parallel, to begin the evolution campaign towards PAM-specific Nme2Cas9 variants using phage assisted non-continuous evolution (PANCE). Although PANCE is generally lower stringency than continuous evolution, the ability to multiplex in 96-well plates enables a high degree of control over evolution conditions. This control was particularly powerful for negative selection, as the rate at which positive or negative stringency should be increased was difficult to predict in advance. There were multiplexed 3 negative selection stringencies with the 4 target PAMs (positive selection: triple PAM, split SAC-PACE) for a total of 12 initial evolution conditions (N1, FIG. 35A). Promisingly, propagation of phage containing PAM-promiscuous variants of Nme2Cas9 (E5 phage) in overnight assays appeared to be directly correlated to the expression level of the APₙ, with higher promoter strengths on AP, resulting in lower propagation (FIG. 35B).

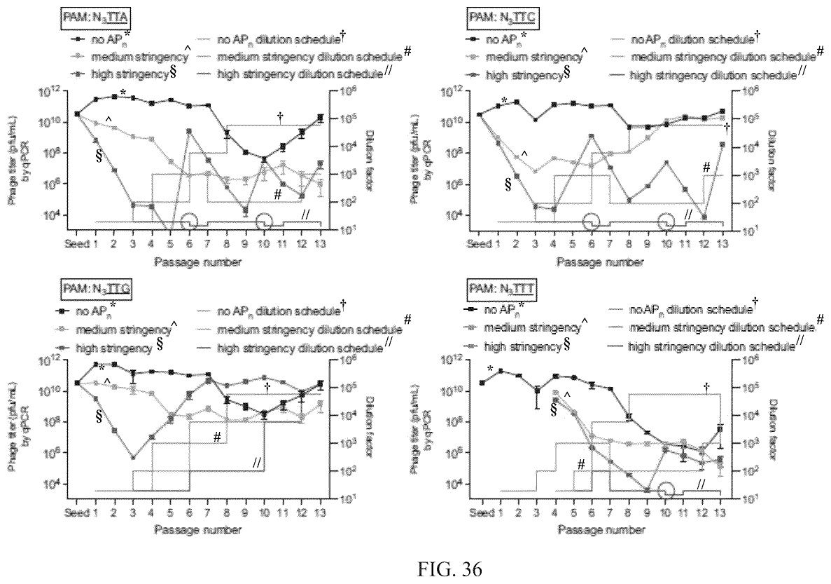

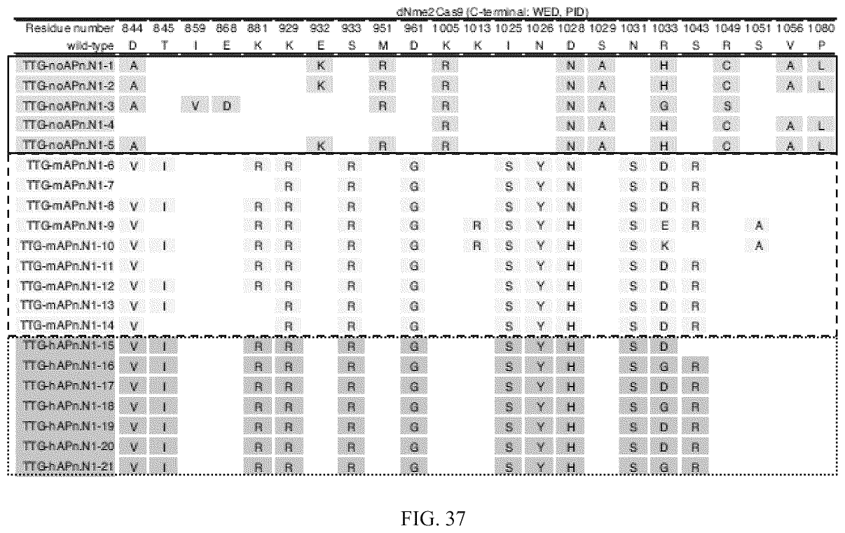

After 13 initial rounds of PANCE, while all positive-only selections exhibited robust phage propagation, most of the high stringency negative selection trajectories were unable to sustain phage propagation except for conditions evolved on N₃TTG PAM-containing APs (FIG. 36). This result would suggest that across the mutations present in the PAM-permissive variants of Nme2Cas9, few enable strong differentiation between N₃CCC and N₃TTN PAMs, unless if the PAM position 6 base is a G. Sequencing was performed on phage from each of the three negative selection stringencies targeting an N₃TTG PAM and found strong inter-lagoon convergence for phage evolved on the same negative selection stringency. Importantly, it was observed observed a drastic shift in the identity of converged mutations within the PAM-interacting domain (PID) that corresponded to the presence of negative selection (FIG. 37).To examine whether these Nme2Cas9 variants subjected to negative selection were indeed exhibiting altered PAM compatibilities, the ABE-PPA was used to profile a representative variant evolved with high stringency negative selection (N1-21-ABE8e) or without negative selection (N1-5-ABE8e) (FIG. 38A). At the targeted N₃TTG PAM, N1-5-ABE8e averaged 75% A•T-to-G•C conversion while N1-21-ABE8e average 67%, indicating comparable but slightly lower activity on the on-target PAM when negative selection is included. In contrast, however, at the negatively selected off-target N₃CCC PAM, the N1-5-ABE8e variant retains 57% A•T-to-G•C conversion, corresponding to a 1.3-to-1 on-to-off target activity ratio, while the N1-21-ABE8e variant virtually eliminates activity at this PAM, averaging 4.8% A•T-to-G•C conversion, or a 14-to-1 on-to-off target activity ratio (FIG. 38B). More broadly, the N1-21-ABE8e variant appears to strongly disfavor sequences with a cytosine at PAM position 5 or 6, so long as the position 6 base is not a guanine. In contrast, the N1-5-ABE8e variant retains the broad PAM promiscuity observed for previous Nme2Cas9 variants evolved towards an N₄TN PAM. These results would suggest that the dual positive/negative SAC-PACE selection can generate more PAM-tailored variants of Nme2Cas9.

Notably, an unintended outcome of this initial dual positive/negative selection was the emergence of an insufficiently limited PAM scope. While the N1-21-ABE8e variant did exhibit reduced PAM promiscuity relative to the N1-5-ABE8e variant, especially on N₄CN and N₄NC PAMs, the former variant exhibited improved activity at G-containing PAMs. At all N₄GN PAMs, N1-21-ABE8e averaged 64% A•T-to-G•C conversion compared to 60% for N1-5-ABE8e. This result would suggest that in the absence of explicit counterselection against all undesired PAM compatibilities, the dual positive/negative selection may not necessarily yield the desired PAM scope. In the case of this N₃TTG trajectory, counterselection against an N₃CCC PAM alone preferentially yielded variants that no longer had activity on N₄CN PAMs, but acquired or retained strong activity on N₄GN PAMs, a group of PAMs that may not be desired. Further optimization of the selection stringency via introduction of a multiple-PAM counterselection could be beneficial for evolving more specific variants.

Next, the N1-21-ABE8e variant was compared to the previously evolved, PAM-promiscuous eNme2-T.1-ABE8e variant in mammalian cells at six genomic sites containing N₃NTN PAMs (FIG. 39). Indeed, the improved specificity of the N1-21-ABE8e variant for N₃NTG PAMs observed in ABE-PPA appears to translate to the initial panel of endogenous genomic sites tested. At the four tested sites containing N₃NTG PAMs, N1-21-ABE8e exhibited comparable or slightly improved adenine base editing activity to eNme2-T.1-ABE8e (16.0% vs. 14.2% A-T-to-G-C conversion). In contrast, at the two tested sites containing N₃NTW PAMs (where W=A or T), N1-21-ABE8e is not active, exhibiting 0.1% A•T-to-G•C conversion compared to the robust activity of eNme2-T.1-ABE8e at these same sites (36.0% A•T-to-G•C conversion). Together, this initial mammalian cell data coupled with the observed PAM profile from ABE-PPA indicates that the dual positive/negative SAC-PACE selection is capable of generating more PAM-specific variants of Cas proteins.

### DISCUSSION

The first evolution of a non-S. pyogenes Cas protein, to acquire single-nucleotide PAM recognition, was demonstrated by integrating a novel functional Cas enzyme selection (SAC-PACE) with high-throughput phage-assisted evolution platforms (PANCE & ePACE) and a high-throughput PAM profiling method (BE-PPA) to guide the evolutionary campaign. Two highly efficient, highly specific Nme2Cas9 variants capable of targeting N₄CN PAM sequences across different gene editing modalities and two variants capable of adenine base editing at many N₄TN PAM sequences were developed, affording unparalleled access to pyrimidine-PAM sequences. Together, these variants complement the suite of commonly used SpCas variants and enable the study and potential correction of previously inaccessible or poorly accessible loci, while retaining the compact size and high genome-wide specificity of Nme2Cas9 that could be beneficial to downstream clinical applications.

In contrast to prior Cas9 evolutions which selected for novel PAM binding^{10,11}, SAC-PACE requires both novel PAM binding and subsequent activation steps necessary for base editing, increasing the likelihood of evolving desired editing properties. In addition to developing this new selection, it was determined that improvements analogous to those made to evolve high-activity SpCas9 variants could be easily incorporated into SAC-PACE, including limiting the concentration of active base editor through a split-intein system and requiring multiple editing events through the inclusion of additional base editing sites. Notably, the evolution campaign that resulted in eNme2-C(SEQ ID NO: 1) generated substantially improved activity on N₄CC PAMs, the PAMs recognized by the wild-type protein, along with numerous mutations outside of the PID that appeared to contribute to this improved activity. This outcome supports the hypothesis that a functional selection enables improved evolution outcomes, in particular for Cas variants with lower starting activity¹⁶. Importantly, these selections should be broadly adaptable to the evolution of any Cas ortholog towards novel PAMs, and the sequence-agnostic nature of the target site can be applied to evolving novel editing windows or disease-specific contexts. The development of ePACE facilitated parallel, automated, and fully continuous evolution of Nme2Cas9 on multiple PAMs, overcoming many of the design, operation, and infrastuctural challenges of traditional PACE and adding to a growing set of automated directed evolution systems^{33,34}. Notably, precise fluidic control was achieved using customizable, millifluidic IPP devices that could be readily and inexpensively manufactured in the lab to automate the fluidic handling needs of PACE, further reducing the need for intervention and enhancing scalability. ePACE can be further customized by modifying the millifluidics and eVOLVER smart sleeves to accommodate fewer chemostats feeding additional lagoons, thereby increasing the potential throughput of ePACE on a single eVOLVER base unit. This would be especially useful for PACE selections in which the same AP can be used while the SP or media conditions are varied across lagoons. Additionally, given the highly reconfigurable nature of eVOLVER, it would be relatively simple to modify the smart sleeves to allow for smaller volumes (˜1 mL) for PACE experiments that rely on expensive media additives to save on costs. Taken together, these technical developments to systematize PACE in a low-cost format coupled with eVOLVER's flexibility for enabling new experimental dimensions will lower the barrier to entry for labs interested in applying PACE.

The selection stringencies were modulated during ePACE experiments based on discrete qPCR phage titer estimations. However, a future prospect for ePACE is to develop and run “algorithmic selection routines” that autonomously adjust selective pressures for individual PACE cultures based on real-time monitoring and feedback from the evolving population. Indeed, it is possible to estimate phage titers in PACE through coupling a luminescence readout to gIII transcription⁵⁵. Additional incorporation of automated feedback based on luminescence in ePACE would further improve the ability to traverse evolutionary landscapes by lowering the lag time between titer readouts and stringency modulation, minimizing the need for researcher interaction and decision-making during experiments.

While ePACE lagoons were provided with the opportunity to evolve activity on specific PAM variants (e.g. four separate lagoons for each N₄CN PAM), variants emerged that were broadly active on the PAM position 5 base that was targeted (C or T). This outcome was expected for selection schemes that select for novel activity but do not counter-select against undesired activities. Nevertheless, predicting which target PAM would yield eNme2-C (SEQ ID NO: 1), eNme2-T.1 (SEQ ID NO: 2), or eNme2-T.2 (SEQ ID NO: 3) a priori likely would have been difficult, as starting activity of wild-type Nme2Cas9 on any N₄CN or N₄TN was comparably low. This challenge highlights the strength of the ePACE platform, which enabled exploration of all trajectories in parallel, greatly enhancing the rate at which high activity variants (five ePACE versus 20 to 40 traditional PACE experiments) could be discovered. Subsequent incorporation of a counter-selection⁵⁵ against undesired PAMs in an ePACE-enabled parallel manner may result in highly PAM- or protospacer-specific Cas variants that further advance tailor-made genome modifying technologies.

### Methods

### General Methods

Antibiotics (Gold Biotechnology) were used at the following working concentrations: carbenicillin—50 μg/mL, chloramphenicol—25 μg/mL, kanamycin—50 μg/mL, tetracycline—10 μg/mL, streptomycin—50 μg/mL. Nuclease-free water (Qiagen) was used for PCR reactions and cloning. All PCR reactions were carried out using Phusion U Hot Start polymerase

(Thermo Fisher Scientific) unless otherwise noted. All plasmids and SP described in this disclosure were cloned by USER assembly unless otherwise noted. Primers and gene fragments used for cloning were ordered from Integrated DNA Technologies (IDT) or Eton Biosciences, as necessary. For cloning purposes, Mach1 (Thermo Fisher Scientific) cells were used, and subsequent plasmid purification was done with plasmid preparation kits (Qiagen or Promega). Illustra TempliPhi DNA Amplification Kits (Cytiva) were used to amplify cloned plasmids prior to Sanger sequencing. For all phage related experiments (phage cloning, phage propagation, PACE and PANCE experiments) were done in parent E. coli strain S2060. A list of protospacer sequences used in this Example is provided in Table 2.

### Overnight Phage Propagation Assay

Chemicompetent S2060 cells were transformed with the AP(s) and CP(s) of interest as previously described. Single colonies were subsequently picked and grown overnight in DRM media with maintenance antibiotics at 37° C. with shaking, then back-diluted 200-1000 fold into fresh DRM media the next day and grown. Upon reaching OD₆₀₀ 0.4-0.6, host cells are transferred into 500 μL aliquots and infected with 10 μL of desired SP (final titer 1×
10⁵ pfu/mL). Cells were then incubated for another 16-20 hours at 37° C. with shaking, then centrifuged at 3,600 g for 10 minutes. The supernatant containing phage was stored until use.

### Plaque Assay

S2060 cells transformed with pJC175e (S22083) were used for plaque assays unless otherwise stated. To prepare a cell stock, an overnight culture of S2208s was diluted 50-fold into fresh 2xYT media with Carbenicillin (50 ug/mL) and grown at 37° C. to an OD₆₀₀˜0.6-0.8. SP were serially diluted (4 dilutions—1:10 first dilution from concentrated phage stocks, then 1:100 remaining 3 dilutions) in DRM. 10 μL of each dilution is added to 150 μL of cells, followed by addition of 850 μL of liquid (55° C.) top agar (2xYT media+0.4% agar) supplemented with 2% Bluo-gal (1:50, final concentration 0.04%, Gold Biotechnology). These mixtures are then pipetted onto one quadrant of a quartered Petri dish containing 2 mL of solidified bottom agar (2xYT media+1.5% agar, no antibiotics). Plates are allowed to briefly solidify before being incubated at 37° C. overnight without inversion.

qPCR Estimation of Phage Titer

When noted, phage titers were estimated by qPCR rather than plaque assay. SP pools (50 μL) were first heated at 80° C. for 30 minutes to destroy polyphage. Polyphage genomes were then degraded by adding 5 μL of heated SP to 45 μL of 1× DNase I buffer containing 1 μL DNase I (New England Biolabs) and incubated at 37° C. for 20 minutes followed by 95° C. for 20 minutes. 1.5 μL of each prepared phage DNA stock is then added to a 25 μL qPCR reaction, prepared as follows: 10.5 μL H₂O, 12.5 μL 2× Q5@ Mastermix (New England Biolabs), 0.25 μL Sybr Green (Thermo Fisher Scientific), 0.125 μL each primer (qPCR-Fw: 5′-CACCGTTCATCTGTCCTCTIT (SEQ ID NO: 30) and qPCR-Rv: 5′-CGACCTGCTCCATGTTACTTAG (SEQ ID NO: 31)). qPCR was then run with the following cycling conditions: 98° C. for 2 minutes, 45 cycles of: [98° C. for 10 seconds, 60° C. for 20 seconds, and 72° C. for 15 seconds]. Titers were calculated using a titration curve of an SP standard of known titer (by plaque assay). A limit of detection was set based on when primers amplified (without SP) or at the lowest titer prior to loss of linearity for the SP standard.

### Phage-Assisted Noncontinuous Evolution

Chemically competent S2060s were transformed with the AP(s) and CP(s) of interest along with a mutagenesis plasmid (MP6⁴¹), and plated on 2xYT agar containing maintenance antibiotics and 100 mM glucose. Three colonies were subsequently picked into DRM with maintenance antibiotics and grown at 37° C. with shaking to an OD₆₀₀˜0.4-0.6. Host cells were then transferred into a 96-well plate in 500 μL aliquots, 10 mM arabinose was added to induce mutagenesis, and SP dilutions from prior passages (or starting phage stocks) were added according to the dilution schedules described in FIGS. 26A-26B and FIGS. 28A-28B for N1 and N2, respectively. Cells were grown for 12-16 hours at 37° C. with shaking, and subsequent SP were isolated in the supernatant following centrifugation at 3,600 g for 10 minutes. To increase and diversify phage titers when necessary, SP were passaged in 52208s containing MP6; during such passages, cells were only infected for 6-8 hours. Starting phage stocks for PANCE1 (N1) and PANCE2 (N2) were all diversified using this method prior to infection into the first PANCE passage. All SP titers were estimated by qPCR as described above.

eVOLVER-Supported Phage-Assisted Continuous Evolution
General ePACE Methods

eVOLVER and PACE were run as previously described^{3,22} with the following modifications. Millifluidic devices controlling inducer flow into lagoons were sterilized before connecting to the vials by filling lines and devices with 10% bleach letting sit for 30 minutes. Bleach was subsequently flushed out with autoclaved di water, then lines purged with air and connected to the vials and inducer bottles. Chemostats were inoculated to OD₆₀₀ 0.05 and run at 30 ml total volume at 1 vol/hour. Cell OD was allowed to reach steady state before flow was initiated into the lagoons. The volume of lagoons was set to 10 mL via continuous pumping of waste with a high flow rate (45 ml/minutes) peristaltic pump (SQ2349291, FynchBio) from a 4″ hypodermic needle (Air-Tite™ N224) set in Port 2 of the custom ePACE vial cap

(FIGS. 14A-14D). Cells were set to pump in through Port 4 using a slow flow rate (1 ml/minute) peristaltic pump (SQ2112453, FynchBio) from a 3″ hypodermic needle (Air-Tite N163), and arabinose was pumped in through Port 1 using an IPP device. Before lagoon infection with phage, cells from the chemostats were flowed through the vessel at 1 vol/hour with 250 mM arabinose flowing at 0.08 vol/hour for at least 1 hour. Upon infection, cell flow rates were changed to the desired rate and arabinose flow rate set to 0.04 vol/hour. Sampling and decisions on flow rate modifications were done as previously described³. Phage titer was quantified via qPCR method described above.

### Millifluidic Fabrication

All IPP and pressure regulator millifluidic devices were constructed as previously described²². Briefly, fluidic designs were drawn out in EAGLE (Autodesk) and patterned onto ¼″ and ⅛″ acrylic using a 40W C02 laser cutter (Epilog Mini 24). The surface of the acrylic was then plasma treated for 1 minute with atmospheric gases at the maximum setting (Harrick Plasma, 30W Expanded Plasma Cleaner) to promote adhesion. These layers were then bonded together using an optically clear laminating adhesive sheet (3M, 8146-3) with a silicone membrane (0.01″, Rogers Corporation, BISCO HT-6240) between them that enables valve actuation.

### IPP Calibrations

To calibrate IPP devices, sealed bottles containing 1 L of water were attached to the input and pressurized to 1.5 psi. IPPs were controlled via 3-way solenoid valves (S10MM-31-12-3, Pneumadyne) connected to the custom eVOLVER pressure regulator system supplying 8 psi (FIGS. 16A-1616E). Pumps were run at 4 different actuation frequencies long enough for at least 100 μl of water to flow, and then measured via pipette. A function of the form y=kx^{a} was then fit to the resulting data and used to calculate the actuation frequency needed for a desired flow rate during experiments.

ePACE1

Host cells transformed with pTPH405 APs (each of the eight N₃YTN PAMs) and MP6 were maintained in a chemostat as described above. Lagoons (8 total, 1 replicate of each PAM) were maintained as described above prior to infection with phage containing full-length wild-type Nme2-ABE8e in the SP391c architecture. Flow rate schedules and titers are found in FIGS. 18A-18B.

ePACE2

Host cells transformed with pTPH405 APs (each of the eight N₃YTN PAMs) and MP6 were maintained in a chemostat as described above. Lagoons (16 total, 2 replicates of each PAM) were maintained as described above prior to infection with pooled surviving phage from ePACE1 lagoons evolved on N₃CTC and N₃TTC PAMs. Flow rate schedules and titers are found in FIGS. 20A-20B.

ePACE3

Host cells transformed with pTPH405c (recoded gIII N-terminus) APs (each of the eight N₃YTN PAMs except N₃TTA PAM), pTPH412 TadA8e R26G-expressing CP, and MP6 were maintained in a chemostat as described above. Lagoons (14 total, 2 replicates of each PAM) were maintained as described above prior to infection with pooled surviving phage from ePACE1 and ePACE2 recoded into the split-phage SP404 architecture. Flow rate schedules and titers are found in FIGS. 24A-24B.

ePACE4

Host cells transformed with pTPH418b (recoded gill N-terminus, dual PAM) APs (each of the six N₃WCD PAMs), pTPH412 TadA8e R260-expressing CP, and MP6 were maintained in a chemostat as described above. Lagoons (16 total) were maintained as described above prior to infection with either pooled N1 replicate 1 & 2 passage 20 phage (6 lagoons), pooled N1 replicate 3 & 4 passage 20 phage (6 lagoons), or pooled N1 replicates 1-4 passage 20 phage (3 lagoons—N₃TCD PAMs). All lagoons were seeded with phage from corresponding N1 PAM lagoons. Flow rate schedules and titers are found in FIGS. 27A-27B.

ePACE5

Host cells transformed with pTPH418b (recoded gIII N-terminus, dual PAM) APs (each of the eight N₃YTN PAMs), pTPH412 TadA8e R26G-expressing CP, and MP6 were maintained in a chemostat as described above. Lagoons (16 total, 2 replicates of each PAM) were maintained as described above prior to infection with pooled N2 replicate 3 passage 7 phage from corresponding PAM lagoons. Flow rate schedules and titers are found in FIGS. 29A-29B.

### Base Editing-Dependent PAM Profiling

### Cloning of BE-PPA Libraries

Cloning of the library plasmids (pTPH342 for CBE-PPA, pTPH424 for ABE-PPA.) was done via one-piece USER assembly of purified PCR product amplified using a primer pool containing all desired PAM sequences (IDT). Purified PCR product was aliquoted into two 0.2 pmol USER reactions (˜500 ng of a 4.2 kb fragment each), purified following USER digestion with PB buffer (Qiagen) and subsequent PE buffer washes (4×, Qiagen), and eluted into 15 μL H₂O. The entire amount was then transformed into electrocompetent 10B cells (New England Biolabs), enough to yield at minimum 14× coverage⁵⁶ of the expected library size. Electroporation was done in 25 μL aliquots using bacterial program X_13 in the 96-well Shuttle Device component of a 4D-Nucleofector system (Lonza). Transformed cells were immediately transferred to 1.5 mL (per 100 μL cells) of prewarmed SOC media. A serial dilution of the transformed cells (8 dilutions, 5-fold each, starting with undiluted cells) was immediately taken and plated on maintenance antibiotics, which was used to calculate effective library size. The remaining cells are allowed to recover at 37° C. with shaking for 1 hour prior to plating on 2xYT agar containing maintenance antibiotic. The following day, colonies were scraped and DNA was isolated using a Plasmid Plus Midi Kit (Qiagen).

### Base Editing-Dependent PAM Profiling Assay

Chemicompetent 10B cells (New England Biolabs) were transformed with the base editor variants of interest. Three colonies of each base editor variant are seeded into 10 mL fresh DRM with maintenance antibiotic and grown at 37° C. with shaking to an OD₆₀₀˜0.4-0.6. Upon reaching the desired cell density, cells were spun down at 5,000×g for 10 minutes, washed 3× with ice-cold 10% (v/v) glycerol, then resuspended in a final volume of 100 μL 10% glycerol. 1 ug of library plasmid (pTPH342 or pTPH424) was added to these 100 μL aliquots, then transformed in 25 μL aliquots using bacterial program X_5 in the 96-well Shuttle Device component of a 4D-Nucleofector system. Transformed cells were immediately transferred to 1.5 mL (per 100 μL cells) of prewarmed SOC media. A serial dilution of the transformed cells (8 dilutions, 5-fold each, starting with undiluted cells) was immediately taken and plated on maintenance antibiotics, which was used to calculate effective library size. The remaining cells are allowed to recover at 37° C. with shaking for 15 minutes, then diluted into 40 mL of prewarmed DRM containing maintenance antibiotics and 10 mM arabinose. Induced cells are then grown at 37° C. with shaking for 22 hours (ABE-PPA), or for 32 hours with a 1:40 back-dilution at 16 hours (CBE-PPA) before being harvested by centrifugation at 3,600×g for 10 minutes. DNA is isolated from harvested cells using a Plasmid Plus Midi Kit (Qiagen).

### High-Throughput DNA Sequencing

Library samples were prepared for high-throughput amplicon sequencing in two PCR steps. The first PCR (PCR1) was performed using forward primer BE-PPA-Fw and reverse primer BE-PPA-Rv at a 150 μL scale and 1 μg of template DNA. Cycling conditions were as follows: 98° C. for 2 minutes, then 14 cycles of [98° C. for 15 seconds, 60° C. for 15 seconds, 72° C. for 20 seconds], and a final extension at 72° C. for 2 minutes. 14 cycles for PCR1 was observed to be within the linear amplification range for the libraries used in this disclosure but may change for alternate library constructions. Following PCR1, PCR reactions were purified using the QIAquick® PCR Purification Kit (Qiagen) and eluted in 16 μL nuclease-free H₂O. The second PCR (PCR2) was performed using forward and reverse Illumina barcoding primers at a 75 μL scale and half (8 μL) of the PCR1 purified product. Cycling conditions were as follows: 98° C. for 2 minutes, then 8 cycles of [98° C. for 15 seconds, 60° C. for 15 seconds, 72° C. for 20 seconds], and a final extension at 72° C. for 2 minutes. 8 cycles for PCR2 was observed to be within the linear amplification range for the libraries used in this disclosure but may change for alternate library constructions. PCR2 products were pooled, purified by electrophoresis with a 1% agarose gel using a QIAquick® Gel Extraction Kit (Qiagen), and eluted in nuclease-free H₂O. DNA concentration was quantified with the KAPA Library Quantification Kit-Illumina® (KAPA Biosystems) and sequenced on an Illumina MiSeq® instrument (paired-end read—R1: 210 cycles, R2: 0 cycles) according to the manufacturer's protocols.

### Analysis of BE-PPA HTS Data

Sequencing reads were demultiplexed using the Miseq® Reporter (Illumina). Demultiplexed files were subsequently analyzed for base editing activity using a custom workflow combining the SeqKit⁵⁷ and CRISPResso2⁵⁸ packages. See Example 2 for additional details. Post-CRISPResso² analyzed nucleotide frequencies are listed in Table 1.

### Cell Culture

HEK293T cells (ATCC CRL-3216), SCD allele containing HEK293T cells⁵⁶ and HUH7 cells were cultured in Dulbecco's modified Eagle's medium plus GlutaMax™ (DMEM, Thermo Fisher Scientific) supplemented with 10% (v/v) fetal bovine serum (FBS, Thermo Fisher Scientific). U20S cells were cultured in McCoy's 5A Medium (Thermo Fisher Scientific) supplemented with 10% (v/v) FBS. Normal adult human primary dermal fibroblasts (HDFa. ATCC PCS-201-012) were cultured in DMEM plus GlutaMax™ supplemented with 20% (v/v) FBS. All cell types were cultured at 37° C. with 5% CO₂. Cell lines were authenticated by their suppliers and tested negative for mycoplasma.

### HEK293T, HUH7, and U2OS Cell Line Transfection Protocols and Genomic DNA Isolation

HEK293T cells were seeded at a density of 2×10⁴ cells per well on 96-well plates (Corning) 16-20 hours prior to transfection. Transfection conditions were as follows for HEK293T cells: 0.5 μL Lipofectamine 2000 (Thermo Fisher Scientific), 250 ng of Cas effector plasmid (nuclease/base editor), and 83 ng of guide RNA plasmid were combined and diluted with Opti-MEM reduced serum media (Thermo Fisher Scientific) to a total volume of 10 μL and transfected according to the manufacturer's protocol. Cells were transfected at approximately 60-80% confluency. HUH7 cells and U20S cells were seeded at a density of 2.5×10⁴ cells per well on 96-well plates 16-20 hours prior to transfection. Transfection conditions were as follows: 0.33 μL Lipofectamine 2000, 112.5 ng of Cas effector plasmid, and 37.5 ng of guide RNA plasmid were combined and diluted with Opti-MEM media to a total volume of 10 μL and transfected according to the manufacturer's protocol. Cells were transfected at approximately 80-100% confluency. Following transfection, all cell types were cultured for 3 days, after which the media was removed, the cells washed with 1×PBS solution, and genomic DNA harvested via cell lysis with 30 μL lysis buffer added per well (10 mM Tris-HCL, pH 8.0, 0.05% SDS, 20 μg/mL Proteinase K (New England Biolabs)). The cell lysis mixture was allowed to incubate for 1-2 hours at 37° C. before being transferred to 96-well PCR plates and enzyme inactivated for 30 minutes at 80° C. The resulting genomic DNA mixture was stored at −20° C. until further use.

Base Editor mRNA In Vitro Transcription

All base editor mRNA was generated from PCR product amplified from a template plasmid containing an expression vector for the base editor of interest cloned as described previously⁵⁹. PCR product was amplified using forward primer IVT-F and reverse primer IVT-R, purified using the QIAquick® PCR Purification Kit (Qiagen), and eluted in 15 μL nuclease-free H₂O. In vitro transcription was done using the HiScribe™ T7 High-Yield RNA Synthesis Kit (New England Biolabs) according to the manufacturer's protocols but with full substitution of N1-methyl-pseudouridine (TriLink Biotechnologies) for uridine and cotranscriptional capping with CleanCap® AG (TriLink Biotechnologies). mRNA isolation was performed using lithium chloride precipitation. Purified mRNA was stored at −20° C. until further use.

### Human Primary Fibroblast Nucleofection and Genomic DNA Extraction

One day prior to nucleofection, 80-90% confluent HDFa cells were passaged at a 1:2 dilution ratio into fresh media. Nucleofection was performed by pooling 1×10⁵ HDFa cells per condition and spun down at 300×g for 10 minutes, washed with 1×PBS, spun again, then resuspended in P2 primary cell solution (10 μL per condition, Lonza). Concurrently, DNA mixtures were prepared by combining 50 pmol of chemically-synthesized guide RNA⁹ (IDT or Synthego) with 1 μg of in vitro transcribed base editor mRNA and P2 primary cell solution into a total volume of 12 μL. For dose titration experiments, the amount of guide RNA was kept fixed, while the total amount of base editor mRNA was varied (125 ng, 250 ng, or 500 ng). Each 10 μL aliquot of HDFa cells is combined with DNA mixture to a total volume of 22 μL, and nucleofected with program DS-150 on 96-well Shuttle Device component of a 4D-Nucleofector system. Following nucleofection, cells were allowed to rest for 10 min before addition of 100 μL prewarmed media per well. 80 μL of each condition was subsequently taken and plated on a 48-well poly-D-lysine plate (Corning). Cells were cultured for 5 days post-nucleofection, with media replacement after the first day. Following removal of media and a wash with 1×PBS buffer, genomic DNA was isolated by addition of 100 μL lysis buffer following the same protocol as described for other cell lines. Genomic DNA was stored at −20° C. until further use.

### High-Throughput Sequencing of Genomic DNA

High-throughput sequencing of genomic DNA from all cell lines was performed as previously described⁹. The sequence identity of the target amplicons are listed in Table 2. DNA concentrations were quantified with a Qubit™ dsDNA High Sensitivity Assay Kit (Thermo Fisher Scientific) or with a NanoDrop™ One Spectrophotometer (Thermo Fisher Scientific) prior to sequencing on an Illumina MiSeq®instrument (paired-end read—R1: 250-280 cycles, R2: 0 cycles) according to the manufacturer's protocols.

### High-Throughput Sequencing Data Analysis

Individual sequencing runs were demultiplexed using the MiSeq® Reporter (Illumina). Subsequent demultiplexed sequencing reads were analyzed using CRISPResso2⁵⁸ as described previously⁹. All editing values are representative of n=3 independent biological replicates, with mean±SEM shown.

### In Silico Prediction of Off-Target Sites

Off-target site prediction in silico was performed using CHOPCHOPv3⁵⁰ and the “Paste Target” functionality with the following parameters: the Site 1 and Site 2 20-nt SpRY protospacers and corresponding 3-nt PAMs were used as search queries; under search options, the Cas9 PAM was set to custom “NNN”, and mismatches within the protospacer was set to 2; self-complementarity parameters were removed; all other parameters were left as default. All resulting off-targets were then further screened manually, and sites with more than one mismatch within the PAM proximal region (£10 bp from the PAM) were removed. Note that as the 23-nt Nme2Cas9 protospacer includes the 20-nt SpRY protospacer, any off-target for the Nme2Cas9 protospacer must also be an off-target for the SpRY protospacer.

### GUIDE-Seq

### U2OS Nucleofection for GUIDE-Seq

One day prior to nucleofection, 80-90% confluent U20S cells were passaged at a 1:2 dilution ratio into fresh media. Nucleofection was performed by pooling 3×10⁵ U2OS cells per condition and spun down at 300×g for 10 minutes, washed with 1×PBS, spun again, then resuspended in SE solution (10 μL per condition, Lonza). Concurrently, DNA mixtures were prepared by combining 750 ng of Cas9 plasmid, 250 ng of guide RNA plasmid, 5 pmol of the GUIDE-seq dsODN⁵¹, and SE solution into a total volume of 12 μL. Each 10 μL aliquot of U20S cells is combined with DNA mixture to a total volume of 22 μL, and nucleofected with program DN-100 on the 96-well Shuttle Device component of a 4D-Nucleofector system. Following nucleofection, cells were allowed to rest for 10 minutes before addition of 100 μL prewarmed media per well. Each condition was then split into two 50 μL aliquots and plated on 24-well plates (Corning). Cells were cultured for 5 days post-nucleofection, with media replacement after the first day. Following removal of media and a wash with 1×PBS buffer, genomic DNA was isolated using the DNAdvance™ Genomic DNA Isolation Kit (Agencourt), following the manufacturer's protocols. Genomic DNA was stored at −20° C. until further use.

### Genomic DNA Preparation and High-Throughput Sequencing for GUIDE-Seq

Genomic DNA was prepared for GUIDE-Seq as previously described⁵¹, with the following modifications. Genomic DNA shearing, end repair, dA-tailing, and adaptor ligation were done in a one-pot mixture using the NEBNext® Ultra II FS DNA Library Prep Kit for Illumina (New England Biolabs), following the manufacturer's protocol for input DNA>100 ng (without size selection) and a desired fragment size distribution between 300-700 bp. During the adaptor ligation step, the manufacturer-suggested NEBNext® Adaptor for Illumina was replaced with the custom GUIDE-Seq Y-adapter⁵¹. DNA purification was done with AMPure XP beads (Beckman Coulter). The subsequent PCR1, PCR2, library quantification, library normalization, and high-throughput sequencing (paired-end Nextera sequencing—R1: 150, I1: 8, 12: 8, R2: 150) steps were done using the primers and protocols from the previously described protocol⁵¹.

### GUIDE-Seq Analysis

Sequencing reads were demultiplexed using the MiSeq® Reporter (Illumina), then processed individually using the GUIDE-Seq analysis software, updated for Python 3 support (github.com/tsailabSJ/guideseq). SpRY variants were analyzed using a mismatch threshold of 8 and an NNN PAM. Nme2Cas9 variants were analyzed using a mismatch threshold of 11 and an NNNNNN PAM. Visualization plots were generated using a custom version of the original script, which has been uploaded to the GitHub repository (github.com/khalillab/guideseq).

### REFERENCES

1. Newby, G. A. et al. Base editing of haematopoietic stem cells rescues sickle cell disease in mice. Nature 595, 295-302, doi:10.1038/s41586-021-03609-w (2021).
2. Koblan, L. W. et al. In vivo base editing rescues Hutchinson-Gilford progeria syndrome in mice. Nature 589, 608-614, doi:10.1038/s41586-020-03086-7 (2021).
3. Miller, S. M., Wang, T. & Liu, D. R. Phage-assisted continuous and non-continuous evolution. Nature protocols 15, 4101-4127, doi:10.1038/s41596-020-00410-3 (2020).
4. Edraki, A. et al. A Compact, High-Accuracy Cas9 with a Dinucleotide PAM for In Vivo Genome Editing. Molecular Cell 73, 714-726.e714. doi:https://doi.org/10.1016/j.molcel.2018.12.003 (2019).
5. Walton, R. T., Christie, K. A., Whittaker, M. N. & Kleinstiver, B. P. Unconstrained genome targeting with near-PAMless engineered CRISPR-Cas9 variants. Science 368, 290, doi:10.1126/science.aba8853 (2020).
6. Jinek, M. et al. A Programmable Dual-RNA-Guided DNA Endonuclease in Adaptive Bacterial immunity. Science 337, 816-821, doi:10.1126/science.1225829 (2012).
7. Cong, L. et al. Multiplex genome engineering using CRISPR/Cas systems. Science (New York, N.Y.) 339, 819-823, doi:10.1126/science.1231143 (2013).
8. Anzalone, A. V., Koblan, L. W., Liu, D. R. Genome Editing with CRISPR-Cas Nucleases, Base Editors. Transposases, and Prime Editors. Nature Biotechnology, submitted (2020).
9. Huang, T. P., Newby, G. A. & Liu, D. R. Precision genome editing using cytosine and adenine base editors in mammalian cells. Nature Protocols 16, 1089-1128, doi:10.1038/s41596-020-00450-9 (2021).
10. Hu, J. H. et al. Evolved Cas9 variants with broad PAM compatibility and high DNA specificity. Nature 556, 57-63, doi:10.1038/nature26155 (2018).
11. Miller, S. M. et al. Continuous evolution of SpCas9 variants compatible with non-G PAMs. Nature Biotechnology, doi:10.1038/s41587-020-0412-8 (2020).
12. Kleinstiver, B. P. et al. Engineered CRISPR-Cas9 nucleases with altered PAM specificities. Nature 523, 481-485, doi:10.1038/nature14592 (2015).
13. Nishimasu, H. et al. Engineered CRISPR-Cas9 nuclease with expanded targeting space. Science 361, 1259-1262, doi:10.1126/science.aas9129 (2018).
14. Walton, R. T., Christie, K. A., Whittaker, M. N. & Kleinstiver, B. P. Unconstrained genome targeting with near-PAMless engineered CRISPR-Cas9 variants. Science 368, 290-296, doi:10.1126/science.aba8853 (2020).
15. Fedorova, I. et al. PpCas9 from Pasteurella pneumotropica—a compact Type II-C Cas9 ortholog active in human cells. Nucleic Acids Research 48, 12297-12309, doi:10.1093/nar/gkaa998 (2020).
16. Mir, A., Edraki, A., Lee, J. & Sontheimer, E. J. Type II-C CRISPR-Cas9 Biology, Mechanism, and Application. ACS Chem Biol 13, 357-365, doi:10.1021/acschembio.7b00855 (2018).
17. Kleinstiver, B. P. et al. Broadening the targeting range of Staphylococcus aureus CRISPR-Cas9 by modifying PAM recognition. Nature Biotechnology 33, 1293-1298, doi:10.1038/nbt.3404 (2015).
18. Kleinstiver, B. P. et al. Engineered CRISPR-Cas12a variants with increased activities and improved targeting ranges for gene, epigenetic and base editing. Nature Biotechnology 37, 276-282, doi:10.1038/s41587-018-0011-0 (2019).
19. Xu, X. et al. Engineered miniature CRISPR-Cas system for mammalian genome regulation and editing. Mol Cell 81, 4333-4345.e4334, doi:10.1016/j.molcel.2021.08.008 (2021).
20. Edraki, A. et al. A Compact, High-Accuracy Cas9 with a Dinucleotide PAM for In Vivo Genome Editing. Mol Cell 73, 714-726.e714, doi:10.1016/j.molcel.2018.12.003 (2019).
21. Liu, Z. et al. Efficient and high-fidelity base editor with expanded PAM compatibility for cytidine dinucleotide. Science China Life Sciences 64, 1355-1367, doi:10.1007/s11427-020-1775-2 (2021).
22. Wong, B. G., Mancuso, C. P., Kiriakov, S., Bashor, C. J. & Khalil, A. S. Precise, automated control of conditions for high-throughput growth of yeast and bacteria with eVOLVER. Nat Biotechnol 36, 614-623, doi:10.1038/nbt.4151 (2018).
23. Esvelt, K. M., Carlson, J. C. & Liu, D. R. A system for the continuous directed evolution of biomolecules. Nature 472, 499-503, doi:10.1038/nature09929 (2011).
24. Shams, A. et al. Comprehensive deletion landscape of CRISPR-Cas9 identifies minimal RNA-guided DNA-binding modules. Nature Communications 12, 5664, doi:10.1038/s41467-021-25992-8 (2021).
25. Richter, M. F., Zhao, K. T., Eton, E., Lapinaite, A., Newby, G. A., Thuronyi, B. W., Wilson, C., Zeng, J., Bauer, D. E., Doudna, J. A, Liu, D. R. Continuous evolution of an adenine base editor with enhanced Cas domain compatibility and activity. Nature Biotechnology, in press (2020).
26. Thuronyi, B. W. et al. Continuous evolution of base editors with expanded target compatibility and improved activity. Nature Biotechnology 37, 1070-1079, doi:10.1038/s41587-019-0193-0 (2019).
27. Shah, N. H. & Muir, T. W. Inteins: Nature's Gift to Protein Chemists. Chem Sci 5, 446-461, doi:10.1039/C3SC52951G (2014).
28. Gogarten, J. P., Senejani, A. G., Zhaxybayeva, O., Olendzenski, L. & Hilario, E. Inteins: structure, function, and evolution. Annu Rev Microbiol 56, 263-287, doi:10.1146/annurev.micro.56.012302.160741 (2002).
29. Zettler, J., Schutz, V. & Mootz, H. D. The naturally split Npu DnaE intein exhibits an extraordinarily high rate in the protein trans-splicing reaction. FEBS Lett 583, 909-914, doi:10.1016/j.febslet.2009.02.003 (2009).
30. Wang, T., Badran, A. H., Huang, T. P. & Liu, D. R. Continuous directed evolution of proteins with improved soluble expression. Nat Chem Biol 14, 972-980, doi:10.1038/s41589-018-0121-5 (2018).
31. Brissette, J. L., Weiner, L., Ripmaster, T. L. & Model, P. Characterization and sequence of the Escherichia coli stress-induced psp operon. J Mol Biol 220, 35-48, doi:10.1016/0022-2836(91)90379-k (1991).
32. Chen, F. et al. Targeted activation of diverse CRISPR-Cas systems for mammalian genome editing via proximal CRISPR targeting. Nat Commun 8, 14958, doi:10.1038/ncomms14958 (2017).
33. DeBenedictis, E. A. et al. Systematic molecular evolution enables robust biomolecule discovery. Nature Methods 19, 55-64, doi:10.1038/s41592-021-01348-4 (2022).
34. Zhong, Z. et al. Automated Continuous Evolution of Proteins in Vivo. ACS Synthetic Biology 9, 1270-1276, doi:10.1021/acssynbio.0c00135 (2020).
35. Grover, W. H., Skelley, A. M., Liu, C. N., Lagally, E. T. & Mathies, R. A. Monolithic membrane valves and diaphragm pumps for practical large-scale integration into glass microfluidic devices. Sensors and Actuators B: Chemical 89, 315-323, doi:https://doi.org/10.1016/S0925-4005(02)00468-9 (2003).
36. Marshall, R. et al. Rapid and Scalable Characterization of CRISPR Technologies Using an E. coli Cell-Free Transcription-Translation System. Mol Cell 69, 146-157.e143, doi:10.1016/j.molcel.2017.12.007 (2018).
37. Jung, C. et al. Massively Parallel Biophysical Analysis of CRISPR-Cas Complexes on Next Generation Sequencing Chips. Cell 170, 35-47.e13, doi:10.1016/j.cell.2017.05.044 (2017).
38. Leenay, R. T. et al. Identifying and Visualizing Functional PAM Diversity across CRISPR-Cas Systems. Mol Cell 62, 137-147, doi:10.1016/j.molcel.2016.02.031 (2016).
39. Arbab, M. et al. Determinants of Base Editing Outcomes from Target Library Analysis and Machine Learning. Cell 182, 463-480.e430, doi:10.1016/j.cell.2020.05.037 (2020).
40. Zhang, Y., Rajan, R., Seifert, H. S., Mondrag6n, A. & Sontheimer, E. J. DNase H Activity of Neisseria meningitidis Cas9. Mol Cell 60, 242-255, doi:10.1016/j.molcel.2015.09.020 (2015).
41. Badran, A. H. & Liu, D. R. Development of potent in vivo mutagenesis plasmids with broad mutational spectra. Nature Communications 6, 8425, doi:10.1038/ncomms9425 (2015).
42. Sun, W. et al. Structures of Neisseria meningitidis Cas9 Complexes in Catalytically Poised and Anti-CRISPR-Inhibited States. Mol Cell 76, 938-952.e935, doi:10.1016/j.molcel.2019.09.025 (2019).
43. Carvajal-Vallejos, P., Pallissé, R., Mootz, H. D. & Schmidt, S. R. Unprecedented rates and efficiencies revealed for new natural split inteins from metagenomic sources. J Biol Chem 287, 28686-28696, doi:10.1074/jbc.M112.372680 (2012).
44. Pinto, F., Thornton, E. L. & Wang, B. An expanded library of orthogonal split inteins enables modular multi-peptide assemblies. Nature Communications 11, 1529, doi:10.1038/s41467-020-15272-2 (2020).
45. Komor, A. C., Kim, Y. B., Packer, M. S., Zuris, J. A. & Liu, D. R. Programmable editing of a target base in genomic DNA without double-stranded DNA cleavage. Nature 533, 420-424, doi:10.1038/nature17946 (2016).
46. Kim, Y. B. et al. Increasing the genome-targeting scope and precision of base editing with engineered Cas9-cytidine deaminase fusions. Nature Biotechnology 35, 371-376, doi:10.1038/nbt.3803 (2017).
47. Gong, S., Yu, H. H., Johnson, K. A. & Taylor, D. W. DNA Unwinding Is the Primary Determinant of CRISPR-Cas9 Activity. Cell Reports 22, 359-371, doi:https://doi.org/10.1016/j.celrep.2017.12.041 (2018).
48. Ivanov, I. E. et al. Cas9 interrogates DNA in discrete steps modulated by mismatches and supercoiling. Proceedings of the National Academy of Sciences 117, 5853-5860, doi:10.1073/pnas.1913445117 (2020).
49. Kleinstiver, B. P. et al. High-fidelity CRISPR-Cas9 nucleases with no detectable genome-wide off-target effects. Nature 529, 490-495, doi:10.1038/nature16526 (2016).
50. Labun, K. et al. CHOPCHOP v3: expanding the CRISPR web toolbox beyond genome editing. Nucleic Acids Research 47, W171-W174, doi:10.1093/nar/gkz365 (2019).
51. Tsai, S. Q. et al. GUIDE-seq enables genome-wide profiling of off-target cleavage by CRISPR-Cas nucleases. Nature Biotechnology 33, 187-197, doi:10.1038/nbt.3117 (2015).
52. Landrum, M. J. et al. ClinVar: improvements to accessing data. Nucleic Acids Res, doi:10.1093/nar/gkz972 (2019).
53. Landrum, M. J. et al. ClinVar: public archive of relationships among sequence variation and human phenotype. Nucleic Acids Res 42, D980-D985, doi:10.1093/nar/gktl 113 (2013).
54. Lennermann, D., Backs, J. & van den Hoogenhof, M. M. G. New Insights in RBM20 Cardiomyopathy. Curr Heart Fail Rep 17, 234-246, doi:10.1007/si 1897-020-00475-x (2020).
55. Carlson, J. C., Badran, A. H., Guggiana-Nilo, D. A. & Liu, D. R. Negative selection and stringency modulation in phage-assisted continuous evolution. Nat Chem Biol 10, 216-222, doi:10.1038/nchembio.1453 (2014).
56. Bosley, A. D. & Ostermeier, M. Mathematical expressions useful in the construction, description and evaluation of protein libraries. Biomol Eng 22, 57-61, doi:10.1016/j.bioeng.2004.11.002 (2005).
57. Shen, W., Le, S., Li, Y. & Hu, F. SeqKit: A Cross-Platform and Ultrafast Toolkit for FASTA/Q File Manipulation. PLOS ONE 11, e0163962, doi:10.1371/journal.pone.0163962 (2016).
58. Clement, K. et al. CRISPResso2 provides accurate and rapid genome editing sequence analysis. Nature Biotechnology 37, 224-226, doi:10.1038/s41587-019-0032-3 (2019).
59. Gaudelli, N. M. et al. Directed evolution of adenine base editors with increased activity and therapeutic application. Nature Biotechnology 38, 892-900, doi:10.1038/s41587-020-0491-6 (2020).

### Example 3

### Supplementary Note 1. ePACE Pressure Regulation

As IPP devices are sensitive to changes in pressure at valves and in connected media bottles, an 8-channel pressure regulator that can be used to regulate these pressures through the eVOLVER framework was developed. The device consists of sets of two proportional valves that can limit air flow from a high-pressure source and a vent at atmospheric pressure. By connecting an electronic pressure gauge to the output of this valve configuration, it is possible to implement proportional-integral-derivative (PID) control over the valves in order to set the output pressure to any desired level between the input and atmospheric pressure. The functionality of this device was validated by regulating pressure at 1.5 psi over 24 hours, and the performance of the device was compared with that of a fixed, manually set regulator (PARKER-WATTS R25-02A) connected to the benchtop air supply (FIGS. 16A-16D). The average pressure with PID control was 1.498 psi with an RMS error of 0.0086 psi, while the fixed regulator had an average pressure of 1.706 psi with an RMS error of 0.2220 psi. Large pressure deviations (>0.5 psi) that can affect the performance of the devices were observed with the fixed regulator, but were successfully eliminated with an automated pressure regulator scheme. The effects of pressure changes were characterized at various locations in the system in order to optimize performance of the IPP devices for the course of a PACE experiment (FIGS. 16A-16D).

### Supplementary Note 2. ePACE2 Recombination and Cheating

During ePACE2, evolving Nme2Cas9 variants on the SP appeared to propagate well in all lagoons on targeted PAMs (each of the eight N3YTN PAMs). Phage were sampled from some lagoons, and the insert was amplified via PCR. Following agarose gel electrophoresis, the SP pools appeared to lose the expected Nme2Cas9 insert, as the resulting bands no longer corresponded to the correct insert size (FIGS. 21A-21C). Sanger sequencing of the incorrectly sized band revealed a region of nucleotide homology between the N-terminus of the gIII construct on the AP and gVI in the phage genome (FIGS. 21B-21C). This site of homology was likely acting as a recombination site enabling some phage to incorporate the gII-C half into the SP genome. As gill-N is constitutively expressed in the original SAC-PACE selection, this enables phage to propagate in a selection genomic siteree manner. For subsequent evolutions, the codon usage of the N-terminus of gill within the AP was altered such that the nucleotide homology was no longer present (pTPH412, Table 5). Following this alteration, recombination was no longer observed.

### Supplementary Note 3. Validation of the Split Base Editor SAC-PACE Selection

To enable control over the expression of active enzyme in the SAC-PACE selection, Nme2ABE8e was split at the linker sequence between TadABE8e and Nme2Cas9. The TadABE8e-half was linked to the N-terminal half of the gp41-8 intein (gp41-8N), and this entire construct (TadABE8e-gp41-8N) was placed on a complementary plasmid (CP) under the control of a psp-promoter and a user-defined ribosome binding sequence. The C-terminal half of the base editor (dNme2Cas9) was linked to the C-terminal half of the gp41-8 intein (gp41-8C), and this construct (gp41-8C-dNme2Cas9) was recloned into the SP architecture (SP404, Table 5). The split SAC-PACE selection was then validated by overnight propagation using new split-SP and host cells containing both AP and CP.

While testing the split SAC-PACE selection, it was important to select a TadA variant with the highest Cas-dependent activity to limit bottlenecking the selection at the deamination step. In addition to TadABE8e, the TadABE8e-R26G point mutant that had converged in prior evolutions was tested (FIGS. 19A, 22A-22C). TadABE8e-R26G enabled 10- to 20 genomic siteold stronger propagation compared to wild-type TadABE8e in a Cas-dependent manner, with no propagation in host-cells lacking Nme2Cas9. Moving forward, the TadABE8e-R26G in split base editor SAC-PACE selections (split SAC-PACE) was utilized.

### Supplementary Note 4. PAM-Specific Activity of ePACE4 Evolved Variants Observed in ABE-PPA

Activity improvements of ePACE4 variants on specific PAMs appeared to be agnostic of the PAM targeted during evolution, with most variants preferring N4CA>N4CC>N4CT>N4CG. The exceptions were variants evolved on the N3ICQ PAM, which exhibited N4CG activity comparable to or better than activity on the other three N₄CD PAMs. This result would suggest that binding of the position 6 G is distinct from binding to the other three nucleobases. In line with this hypothesis, the mutation profiles in the PID are relatively conserved between variants evolved on the N3ICA and N3IC APs (S933R, R1033S/G, Q1047R): however, additional mutations outside of the three seen in those variants converged in the N33T trajectory (D873V, E932K, D961G, N1031D/S, K1044R, E1045A, K1077E) (FIG. 7E). However, the difference in activity between the variants was nuanced, as the overall trend reflects general improvements to activity on all N₄CN PAMs (FIG. 7D).

### Supplementary Note 5. Reversion Analysis of eNme2-C RuvC/HNH Domain Mutations

Simple reversion of the RuvC-inactivating mutation D16A in eNme2-C(SEQ ID NO: 1) did not yield a robust nuclease Cas9. Upon reversion of the eight mutations in the RuvC/HNH domains and their associated linker regions to their wild-type residues, the resulting variant eNme2-C.NR (SEQ ID NO: 4) had robust nuclease activity across N₄CN PAM sites. However, reversion of these mutations had a detrimental effect on base editing activity, as the ABE8e version of eNme2-C.NR (SEQ ID NO: 4) had a 1.8 genomic siteold reduction in adenine base editing activity relative to eNme2-C-ABE8e (FIG. 11E). These results would suggest that some or all the mutations in the RuvC/HNH domains are important for Nme2Cas9 activities necessary for base editing, but detrimental to the subsequent activation or catalytic activity of Nme2Cas9 nuclease.

To further explore this idea and to potentially find an optimal dual base editor/nuclease variant, a set of eight single-point reversion variants of mutations in the RuvC/HNH domains of eNme2-C(SEQ ID NO: 1) were generated and evaluated as nucleases and ABEs (FIGS. 11E-I1F). Only two of the eight single-point reversion variants, eNme2-C V696F and eNme2-C R7110, showed significant rescue of nuclease activity (12.5- and 4.4 genomic siteold improvement over eNme2-C, respectively). Conversely, most of the reversions reduced ABE efficiency relative to eNme2-C-ABE8e. Notably, none of the eight variants outperformed eNme2-C(SEQ ID NO: 1) as an ABE or eNme2-C.NR (SEQ ID NO: 4) as a nuclease, highlighting the importance of the amino acid identities at these RuvC/HNH positions in differentiating between base editor and nuclease activities of evolved Nme2Cas9.

### Supplementary Note 6. Analysis and Limitations of BE-PPA for Evaluating Nme2Cas9 PAM Compatibility

All Nme2Cas9 variants (wild-type and evolved) were profiled using ABE-PPA on a single protospacer (“ABE-PPA”, see Table 2) flanked by 512 unique PAMs (pTPH424, see Supplementary Tables 1 and 5). The 512 unique PAMs are only a subset of the theoretical PAM space potentially encompassed by Nme2Cas9 (a six base pair region encompasses 4,096 targetable sequences). The library was designed to observe PAM compatibilities primarily at PAM positions 4-7 (NNNNNNN, 256 combinations), and it was hypothesized that the positions that would most likely alter their nucleotide preference during evolution are the positions canonically recognized by the wild-type enzyme (PAM positions 5 and 6, ±1 base). Two groups of sequences at PAM positions 1-3 (ACGNNNN or ATNNNN), giving the total 512 PAM sequences, although these positions were pooled during analysis. The library size was limited to 512 members for throughput-purposes, as this number allows for profiling of up to 8 variants on an Illumina MiSeq kit (15 m reads, 1.9 m reads per variant, ˜4,000 reads per PAM assuming equal distribution). By limiting the analysis to these positions, it is possible that the PAM compatibility observed is biased by the identity of the bases chosen for positions 1-3, the selected protospacer, or the target adenine position. A larger library may be useful for more comprehensive PAM profiling of final variants. Nevertheless, the subset library provided a rapid, high-throughput method for quickly filtering evolved variants with desired PAM compatibilities and high efficiencies.

TABLE 5
Plasmid and Selection Phage (SP)

| Name | Usage (resistance) | Origin | ORF1 (prom [RBS] genes) | ORF2 (pram [RBS] genes) |
|---|---|---|---|---|
| pTPH353e | AP validation (carb^{R}) | SC101 | P [symbol] [SD8 [symbol] ] gIII(1-18)-NpuN-32aa | |
| | | | linker-NpuC-gIII(18-425), luxAB | |
| pTPH353e-d | AP validation (carb^{R}) | SC101 | P [symbol] [SD8 [symbol] ] gIII(1-18)-NpuN-32aa | |
| | | | linker-NpuC(C1A)-gIII(18-425), luxAB | |
| pTPH353e-d stops | AP validation (carb^{R}) | SC101 | P [symbol] [SD8 [symbol] ] gIII(1-18)-NpuN-32aa linker | |
| | | | (double stop codon)-NpuC-gIII(18-425), | |
| | | | luxAB | |
| pTPH401 | AP validation (carb^{R}) | ColE1 | P_{ProC}⁷ [sd8 [symbol] ] gIII(1-18)-NpuN-64aa | P [symbol] Evo-1 sgRNA |
| | | | linker(Evo-1 protospacer, NNNNCC | |
| | | | PAM)-NpuC-gIII(18-425), luxAB | |
| pTPH400 | AP validation (carb^{R}) | ColE1 | P_{ProC}⁷ [sd8 [symbol] ] gIII(1-18)-NpuN-121aa | P [symbol] Evo-1 sgRNA |
| | | | linker(Evo-1 protospacer, NNNNCC | |
| | | | PAM)-NpuC-gIII(18-425), luxAB | |
| pTPH397 | AP validation (carb^{R}) | ColE1 | P_{ProC}⁷ [sd8 [symbol] ] gIII(1-18)-NpuN-32aa | P [symbol] Evo-1 sgRNA |
| | | | linker(Evo-1 protospacer, NNNNCC | |
| | | | PAM)-NpuC-gIII(18-425), luxAB | |
| pTPH397b | AP validation (carb^{R}) | ColE1 | P_{ProC}⁷ [sd8⁸] gIII(1-18)-NpuN-32aa | P [symbol] Evo-2 sgRNA |
| | | | linker(Evo-2 protospacer, NNNNCC | |
| | | | PAM)-NpuC-gIII(18-425), luxAB | |
| pTPH397c | AP validation (carb^{R}) | ColE1 | P_{ProC}⁷ [sd8 [symbol] ] gIII(1-18)-NpuN-32aa | P [symbol] Evo-3 sgRNA |
| | | | linker(Evo-3 protospacer, NNNNCC | |
| | | | PAM)-NpuC-gIII(18-425), luxAB | |
| pTPH40 [symbol] b | AP: ePACE1-2 (carb^{R}) | ColE1 | P_{ProC}⁷ [sd8 [symbol] ] gIII(1-18)-NpuN-32aa | P [symbol] Evo-2 sgRNA |
| | | | linker(Evo-2 protospacer, varied | |
| | | | PAM [symbol] )-NpuC-gIII(18-425), luxAB | |
| pTPH405c | AP: ePACE3 (carb^{R}) | ColE1 | P_{ProC}⁷ [sd8 [symbol] ] gIII(1-18 recoded)-NpuN- | P [symbol] Evo-2 sgRNA |
| | | | 32aa linker(Evo-2 protospacer, varied | |
| | | | PAMa)-NpuC-gIII(18-425), luxAB | |
| pTPH412 WT sd8 | CP validation (kan^{R}) | SC101 | P [symbol] [sd8 [symbol] ] TadABESe-gp41-8N | |
| pTPH412 WT sd2 | CP validation (kan^{R}) | SC101 | P [symbol] [sd2 [symbol] ] TadABESe-gp41-8N | |
| pTPH412 R26G sd8 | CP: ePACE3-5 (kan^{R}) | SC101 | P [symbol] [sd8 [symbol] ] TadABESe(R26G)-gp41-8N | |
| pTPH412 R26G sd2 | CP validation (kan^{R}) | SC101 | P [symbol] [sd2 [symbol] ] TadABESe(R26G)-gp41-8N | |
| pTPH418b | AP: ePACE4-5 (carb^{R}) | ColE1 | P_{ProC}⁷ [sd8 [symbol] ] gIII(1-18 recoded)-NpuN- | P [symbol] Evo-4 sgRNA |
| | | | 45aa linker(2 x Evo-4 protospacer, | |
| | | | varied PAMs)-NpuC-gIII(18-425), luxAB | |
| pTPH343c | CBE-PPA ( [symbol] pac^{R}) | ColE1 | P [symbol] [SD8 [symbol] ] rAPOBEC1-dSpC [symbol] 9-UGI | P [symbol] CBE-PPA sgRNA |
| pTPH342 | CBE-PPA library (carb^{R}) | SC101 | CBE-PPA protospacer | P [symbol] [S [symbol] ] sfGFP |
| | | | | (inactive) |
| pTPH413 | ABE-PPA (cm [symbol] ) | p15A | P [symbol] [sd4u [symbol] ] TadABE8e-dNme2Cas9 | P [symbol] ABE-PPA sgRNA |
| | | | variant | |
| pTPH424 | ABE-PPA library (carb^{R}) | SC101 | ABE-PPA protospacer | P [symbol] [SD [symbol] ] sfGFP |
| | | | | (inactive) |
| N [symbol] -IVT | IVT template (carb^{R}) | pUC | P [symbol] Nm [symbol] 2ABE8e variant | |
| SpRY-IVT | IVT template (carb^{R}) | pUC | P [symbol] SpRVABE8e or SpRY-HF1- | |
| | | | ABE8e variant | |
| Nme sgRNA | Mammalian guide expression (carb^{R}) | pUC | P [symbol] Nme2Cas9 sgRNA | |
| SpRY sgRNA | Mammalian guide expression (carb^{R}) | pUC | P [symbol] SpRY/SpRY-HF1 sgRNA | |
| Nme2ABE8e | Mammalian expression of adenine | pUC | P_{CMV} Nme2ABE8e variant | |
| variants | base editor (carb^{R}) | | | |
| SpRYABE8e | Mammalian expression of adenine | pUC | P_{CMV} SpRY/SpRY-HF1-ABE8e variant | |
| variants | base editor (carb^{R}) | | | |
| Nme2ABE4 variants | Mammalian expression of cytosine | pUC | P_{CMV} Nme2BE4 variant | |
| | base editor (carb^{R}) | | | |
| SpRYBE4 variants | Mammalian expression of adenine | pUC | P_{CMV} SpRY/SpRY-HF1-BE4 variant | |
| | base editor (carb^{R}) | | | |
| Nme2 nuclease | Mammalian expression of nuclease | pUC | P_{CMV} Nme2Cas9 nuclease variant | |
| variants | base editor (carb^{R}) | | | |
| SpRY nuclease | Mammalian expression of nuclease | pUC | P_{CMV} SpRY/SpRY-HF1 nuclease variant | |
| variants | base editor (carb^{R}) | | | |
| SP391c | ePACE1-2, ΔgIII SP, backbone | M13 [symbol] | P [symbol] [SD4 [symbol] ] TadABE8e-dNme2Cas9 | |
| | recorded [symbol] | | | |
| SP40A | ePACE4-5, ΔgIII SP, backbone | M13 [symbol] | P [symbol] [SD4 [symbol] ] gp41-C-dNme2Cas9 variant | |
| | recorded [symbol] | | | |

[symbol]

indicates data missing or illegible when filed

To analyze BE-PPA sequenced files, the demultiplexed fastq files were filtered using the seqkit package/grep function¹¹ to search for two flank sequences near either end of the amplicon. For ABE-PPA profiled variants, groups of PAMs were UMI-tagged, and the specific UMI tag was used in place of one of the flank sequences. Filtered files were then binned into individual fastq files per PAM using the same function. The resulting PAM-specific fastq files were analyzed using standard CRISPResso2¹² analysis.

### Supplementary Note 7. Design Error for the N₄CN Trajectory Dual PAM Split SAC-PACE APs

When designing the dual PAM split SAC-PACE APs (pTPH418b, see Table 2), the identity of PAM positions 1-3 were set as CTT and AGG for the two target PAMs, both of which fall on the non-coding strand. The TT nucleotides of the CTT-containing PAM occupy codon positions two and three for an arbitrary codon within the AP linker. Notably, when the target PAM is designed to be 3′-CTTACN-5′, the 3′-TTA-5′ nucleotides introduce an additional stop codon in the PAM (5′-TAA-3′ on coding strand), preventing proper correction of the AP. As such, none of the dual PAM split SAC-PACE APs containing an A at PAM position 4 were able to support phage propagation, as observed.

Exemplary guide RNA-encoding nucleic acid sequences comprising a Nme2Cas9 scaffold sequence and spacer sequence are provided below. These sequences comprise SEQ ID NO: 100 fused to one of SEQ ID NOs: 101-106 (indicated in italics). In some embodiments, the guide RNA-encoding sequences of the disclosure comprise any one of the spacer sequences shown below in Table 2, fused to SEQ ID NO: 100.

(SEQ ID NO: 200)
GTTCCTCACTACTCAGAGCCAGAGTTGTAGCTCCCTTTCTCATT
TCGGAAACGAAATGAGAACCGTTGCTACAATAAGGCCGTCTGAA
AAGATGTGCCGCAACGCTCTGCCCCTTAAAGCTTCTGCTTTAAG
GGGCATCGTTTA
(SEQ ID NO: 201)
TGTTCCTCACTACTCAGAGCCAGGTTGTAGCTCCCTTTCTCATT
TCGGAAACGAAATGAGAACCGTTGCTACAATAAGGCCGTCTGAA
AAGATGTGCCGCAACGCTCTGCCCCTTAAAGCTTCTGCTTTAAG
GGGCATCGTTTA
(SEQ ID NO: 202)
TTCCTCACTACTCAGAGCCAGAGGTTGTAGCTCCCTTTCTCATT
TCGGAAACGAAATGAGAACCGTTGCTACAATAAGGCCGTCTGAA
AAGATGTGCCGCAACGCTCTGCCCCTTAAAGCTTCTGCTTTAAG
GGGCATCGTTTA
(SEQ ID NO: 203)
TGTTCCGCCCTACTCAGAGCCAGGTTGTAGCTCCCTTTCTCATT
TCGGAAACGAAATGAGAACCGTTGCTACAATAAGGCCGTCTGAA
AAGATGTGCCGCAACGCTCTGCCCCTTAAAGCTTCTGCTTTAAG
GGGCATCGTTTA
(SEQ ID NO: 204)
GGCCCAGACTGAGCACGTGAGTTGTAGCTCCCTTTCTCATTTCG
GAAACGAAATGAGAACCGTTGCTACAATAAGGCCGTCTGAAAAG
ATGTGCCGCAACGCTCTGCCCCTTAAAGCTTCTGCTTTAAGGGG
CATCGTTTA
(SEQ ID NO: 205)
ATGGCCCAGACTGAGCACGTGAGGTTGTAGCTCCCTTTCTCATT
TCGGAAACGAAATGAGAACCGTTGCTACAATAAGGCCGTCTGAA
AAGATGTGCCGCAACGCTCTGCCCCTTAAAGCTTCTGCTTTAAG
GGGCATCGTTTA

TABLE 2
list of target sites and spacer sequences

| | | | | SEQ | | PAM | |
|---|---|---|---|---|---|---|---|
| Site | Cas9 | Guide | Spacer Sequence | ID | Spacer | (3 or | Locus/ |
| Name | variant | ID | | NO: | Length | 6 nt) | gene |
| Evo-1 | Nme2Cas9 | — | GTTCCTCACTACTCAGA | 101 | 23 | varies | — |
| | | | GCCAGA | | | | |
| Evo-2 | Nme2Cas9 | — | TGTTCCTCACTACTCAGA | 102 | 23 | varies | — |
| | | | GCCAG | | | | |
| Evo-3 | Nme2Cas9 | — | TTCCTCACTACTCAGAG | 103 | 23 | varies | — |
| | | | CCAGAG | | | | |
| Evo-4 | Nme2Cas9 | — | TGTTCCGCCCTACTCAG | 104 | 23 | varies | — |
| | | | AGCCAG | | | | |
| CBE- | SpCas9 | — | GGCCCAGACTGAGCAC | 105 | 20 | varies | — |
| PPA | | | GTGA | | | | |
| ABE- | Nme2Cas9 | — | ATGGCCCAGACTGAGCA | 106 | 22 | varies | — |
| PPA | | | CGTGA | | | | |
| ACC-1 | Nme2Cas9 | Nme1 | GCGCAAAGCTGCATCCA | 187 | 24 | AGGACC | LINC01588 |
| | | | CCCCCCG | | | | |
| ACC-2 | Nme2Cas9 | Nme2 | GACCAGCCCCTCGAAGG | 188 | 24 | AGGACC | LINC01588 |
| | | | CAAGGCC | | | | |
| CCC-1 | Nme2Cas9 | Nme3 | GCAAGAGCACAAGAGG | 189 | 23 | AGACCC | GAPDH |
| | | | AAGAGAG | | | | |
| CCC-2/ | Nme2Cas9 | Nme4 | GCCCTCATCTCCAATAT | 190 | 24 | CGGCCC | SEC618 |
| Site 6 | | | GGTATGG | | | | |
| TCC-1 | Nme2Cas9 | Nme5 | TATGTTCCAGCTTCCTG | 191 | 24 | AGGTCC | LSP1 |
| | | | GGTCTGC | | | | |
| TCC-2 | Nme2Cas9 | Nme6 | GAGGCAAGAGGGGGGC | 192 | 24 | GGGTCC | FANCF |
| | | | TTTGGGCG | | | | |
| ACA-1 | Nme2Cas9 | Nme7 | GATTGCGTACTTCGAAA | 107 | 23 | GCGACA | MECP2 |
| | | | AGGTAG | | | | |
| ACA-2 | Nme2Cas9 | Nme8 | GTTAGCAGACCCAGATA | 108 | 22 | GAGACA | FANCF |
| | | | GACAG | | | | |
| ACA-3 | Nme2Cas9 | Nme9 | GCCCTGCCCACACCCCC | 109 | 22 | ACCACA | LSP1 |
| | | | ACTCC | | | | |
| CCA-1 | Nme2Cas9 | Nme10 | GACCCTAGCCTAATGCC | 110 | 23 | ACCCCA | LSP1 |
| | | | CCCTGC | | | | |
| GCA-1 | Nme2Cas9 | Nme11 | GGTCCAAAGCAGGATG | 193 | 23 | GGGGCA | Intergenic |
| | | | ACAGGCA | | | | |
| GCA-2 | Nme2Cas9 | Nme12 | GTTCGAGGACCATGAG | 194 | 24 | CGCGCA | SHANK3 |
| | | | ATAGAAGG | | | | |
| GCA-3/ | Nme2Cas9 | Nme13 | GCCAGGGCTGGAGAAG | 195 | 23 | AAAGCA | LINC01509 |
| Site 4 | | | CAGAAAA | | | | |
| TCA-1 | Nme2Cas9 | Nme14 | GGCTACAGCAACAGGG | 196 | 22 | ACCTCA | GAPDH |
| | | | TGGTGG | | | | |
| TCA-2 | Nme2Cas9 | Nme15 | GAGCACAAGAGGAAGA | 197 | 22 | CCCTCA | GAPDH |
| | | | GAGAGA | | | | |
| ACG-1 | Nme2Cas9 | Nme16 | GTCCCCAGCCCAAACCG | 198 | 24 | GCGACG | LINC01588 |
| | | | CCGCGGC | | | | |
| ACG-2 | Nme2Cas9 | Nme17 | GATATGGTCCAGTAGAT | 199 | 23 | GTTACG | CUL3 |
| | | | ATTGAA | | | | |
| CCG-1/ | Nme2 Cas9 | Nme18 | GTCTCCGCTTTAACCCCC | 207 | 23 | CAGCCG | Intergenic |
| Site 5 | | | ACCTC | | | | |
| CCG-2 | Nme2Cas9 | Nme19 | GACCCAGGGGTAGAAA | 208 | 24 | GTCCCG | EMX1 |
| | | | TGGAGAGG | | | | |
| CCG-3 | Nme2Cas9 | Nme20 | GAATGGGGCCATGCCG | 209 | 22 | GCGCCG | FANCF |
| | | | ACCAAA | | | | |
| CCG-4 | Nme2Cas9 | Nme21 | GCTACCAGCCCCCACTG | 210 | 24 | GAGCCG | EMX1 |
| | | | GCCCTCA | | | | |
| GCG-1 | Nme2Cas9 | Nme22 | GGTGTGCAGACGGCAG | 211 | 23 | GGGGCG | VEGFA |
| | | | TCACTAG | | | | |
| GCG-2 | Nme2Cas9 | Nme23 | GCCGACCAAAGCGCCG | 212 | 22 | GTGGCG | FANCF |
| | | | ATGGAT | | | | |
| GCG-3 | Nme2Cas9 | Nme24 | GCGGCCGGATATCAGCT | 213 | 23 | CCCGCG | LINC01588 |
| | | | GCCACG | | | | |
| GCG-4 | Nme2Cas9 | Nme25 | GCCGACCAAAGCGCCG | 212 | 22 | GTGGCG | FANCF |
| | | | ATGGAT | | | | |
| TCG-1 | Nme2Cas9 | Nme26 | GCCAAGAAGTCGCCCAT | 215 | 23 | AGCTCG | SHANK3 |
| | | | CGCAGC | | | | |
| ACT-1 | Nme2Cas9 | Nme27 | GGGTGGGGTCAGACGT | 216 | 23 | CAGACT | Intergenic |
| | | | CCAAAAC | | | | |
| CCT-1 | Nme2Cas9 | Nme28 | GCTCATCTCCAATATGG | 217 | 23 | GGCCCT | SEC61B |
| | | | TATGGC | | | | |
| GCT-1 | Nme2Cas9 | Nme29 | GCACAACCAGTGGAGG | 218 | 23 | GCGGCT | FANCF |
| | | | CAAGAGG | | | | |
| GCT-2 | Nme2Cas9 | Nme30 | GAAATGGCACACACATC | 219 | 23 | GCAGCT | MECP2 |
| | | | CCTCGT | | | | |
| GCT-3 | Nme2Cas9 | Nme31 | GCTGCGGAACCGCTTCT | 220 | 23 | ACCGCT | EMX1 |
| | | | TCGGCC | | | | |
| TCT-1/ | Nme2Cas9 | Nme32 | GGCGCAAAGCAGGAGG | 221 | 23 | GCTTCT | SEC618 |
| Site 3 | | | AGTCAGG | | | | |
| TCT-2 | Nme2Cas9 | Nme33 | GGAGGATGAAACAATG | 222 | 22 | CGCTCT | MECP2 |
| | | | TCTTTG | | | | |
| CTA-1 | Nme2Cas9 | Nme34 | GAGGGGAGAAGAAAGA | 223 | 24 | GGGCTA | RUNX1 |
| | | | GAGATGTA | | | | |
| GTA-1 | Nme2Cas9 | Nme35 | GCAGAGCAAATACCAG | 224 | 23 | AGAGTA | GRIN2B |
| | | | AGATAAG | | | | |
| GTA-2 | Nme2Cas9 | Nme36 | GTGTGGTTCCAGAACCG | 225 | 23 | AAAGTA | EMX1 |
| | | | GAGGAC | | | | |
| CTC-1 | Nme2Cas9 | Nme37 | GTTGGCTACAGCAACAG | 226 | 24 | GACCTC | GAPDH |
| | | | GGTGGTG | | | | |
| CTC-2 | Nme2Cas9 | Nme38 | GTGGTGGACCTCATGGC | 227 | 23 | GGCCTC | LSP1 |
| | | | CCACAT | | | | |
| CTC-3 | Nme2Cas9 | Nme39 | GGAACTGGACCCCGCCC | 228 | 23 | GCCCTC | SEC61B |
| | | | AAAGCC | | | | |
| CTC-4 | Nme2 Cas9 | Nme40 | GATATGGGCTATTTAAA | 229 | 23 | TATCTC | CUL3 |
| | | | AATACG | | | | |
| TTC-1 | Nme2Cas9 | Nme41 | GGGGTCCAGTTCCGGG | 230 | 23 | AACTTC | FANCF |
| | | | ATTAGCG | | | | |
| TTC-2 | Nme2Cas9 | Nme42 | GTCAGAAAAAGGGCCC | 231 | 24 | CTTTTC | GAPDH |
| | | | TGACAACT | | | | |
| TTC-3 | Nme2Cas9 | Nme43 | GCCTGCACCCCATGOCT | 232 | 24 | AGCTTC | LSP1 |
| | | | ATGTTCC | | | | |
| ATG-1 | Nme2Cas9 | Nme44 | GTCCAGCAGAGGGGAG | 233 | 24 | GAGATG | RUNX1 |
| | | | AAGAAAGA | | | | |
| CTG-1 | Nme2Cas9 | Nme45 | ATATTGGAGATGAGGG | 234 | 23 | GCGCTG | SEC618 |
| | | | TGGCAAG | | | | |
| GTG-1 | Nme2Cas9 | Nme46 | GCCCAGATAGACAGGA | 235 | 24 | TGGGTG | FANCF |
| | | | GACAGCGC | | | | |
| TTG-1 | Nme2Cas9 | Nme47 | GATCATGGAAGGGCCG | 236 | 23 | ATATTG | SEC61B |
| | | | CCATACC | | | | |
| ATT-1 | Nme2Cas9 | Nme48 | GTGGGCTGTAACAGGA | 237 | 23 | GAGATT | GRIN2B |
| | | | GGGCCAG | | | | |
| TTT-1 | Nme2Cas9 | Nme49 | GGAAAAAGGCCCCAGA | 238 | 23 | TGGTTT | Intergenic |
| | | | AGGAGTC | | | | |
| CCC-1 | Nme2Cas9 | Nme3- | GCAGCAAGAGCACAAG | 239 | 26 | AGACCC | GAPDH |
| | | 26 | AGGAAGAGAG | | | | |
| CCC-1 | Nme2Cas9 | Nme3- | GAGCAAGAGCACAAGA | 240 | 25 | AGACCC | GAPDH |
| | | 25 | GGAAGAGAG | | | | |
| CCC-1 | Nme2Cas9 | Nme3- | GGCAAGAGCACAAGAG | 241 | 24 | AGACCC | GAPDH |
| | | 24 | GAAGAGAG | | | | |
| CCC-1 | Nme2Cas9 | Nme3- | GAAGAGCACAAGAGGA | 242 | 22 | AGACCC | GAPDH |
| | | 22 | AGAGAG | | | | |
| CCC-1 | Nme2Cas9 | Nme3- | GAGAGCACAAGAGGAA | 243 | 21 | AGACCC | GAPDH |
| | | 21 | GAGAG | | | | |
| CCC-1 | Nme2Cas9 | Nme3- | GGAGCACAAGAGGAAG | 244 | 20 | AGACCC | GAPDH |
| | | 20 | AGAG | | | | |
| GCT-2 | Nme2Cas9 | Nme30 | GTGGAAATGGCACACA | 245 | 26 | GCAGCT | MECP2 |
| | | -26 | CATCCCTOGT | | | | |
| GCT-2 | Nme2Cas9 | Nme30 | GGGAAATGGCACACAC | 246 | 25 | GCAGCT | MECP2 |
| | | -25 | ATCCCTCGT | | | | |
| GCT-2 | Nme2Cas9 | Nme30 | GGAAATGGCACACACAT | 247 | 24 | GCAGCT | MECP2 |
| | | -24 | CCCTCGT | | | | |
| GCT-2 | Nme2Cas9 | Nme30 | GAATGGCACACACATCC | 248 | 22 | GCAGCT | MECP2 |
| | | -22 | CTCGT | | | | |
| GCT-2 | Nme2Cas9 | Nme30 | GATGGCACACACATCCC | 249 | 21 | GCAGCT | MECP2 |
| | | -23 | TCGT | | | | |
| GCT-2 | Nme2Cas9 | Nme30 | GTGGCACACACATCCCT | 250 | 20 | GCAGCT | MECP2 |
| | | -20 | CGT | | | | |
| GCA-1 | Nme2Cas9 | Nme11 | GAGGGTCCAAAGCAGG | 251 | 26 | GGGGCA | Intergenic |
| | | -26 | ATGACAGGCA | | | | |
| GCA-1 | Nme2Cas9 | Nme11 | GGGGTCCAAAGCAGGA | 252 | 25 | GGGGCA | Intergenic |
| | | -25 | TGACAGGCA | | | | |
| GCA-1 | Nme2Cas9 | Nme11 | GGGTCCAAAGCAGGAT | 253 | 24 | GGGGCA | Intergenic |
| | | -24 | GACAGGCA | | | | |
| GCA-1 | Nme2Cas9 | Nme11 | GTCCAAAGCAGGATGA | 254 | 22 | GGGGCA | Intergenic |
| | | -22 | CAGGCA | | | | |
| GCA-1 | Nme2Cas9 | Nme11 | GCCAAAGCAGGATGAC | 255 | 21 | GGGGCA | Intergenic |
| | | -21 | AGGCA | | | | |
| GCA-1 | Nme2Cas9 | Nme11 | GCAAAGCAGGATGACA | 256 | 20 | GGGGCA | Intergenic |
| | | -20 | GGCA | | | | |
| CTC-1 | Nme2Cas9 | Nme37 | GATTTGGCTACAGCAAC | 257 | 26 | GACCTC | GAPDH |
| | | -26 | AGGGTGGTG | | | | |
| CTC-1 | Nme2Cas9 | Nme37 | GTTTGGCTACAGCAACA | 258 | 25 | GACCTC | GAPDH |
| | | -25 | GGGTGGTG | | | | |
| CTC-1 | Nme2Cas9 | Nme37 | GTGGCTACAGCAACAG | 259 | 23 | GACCTC | GAPDH |
| | | -23 | GGTGGTG | | | | |
| CTC-1 | Nme2Cas9 | Nme37 | GGGCTACAGCAACAGG | 260 | 22 | GACCTC | GAPDH |
| | | -22 | GTGGTG | | | | |
| CTC-1 | Nme2Cas9 | Nme37 | GGCTACAGCAACAGGG | 261 | 21 | GACCTC | GAPDH |
| | | -21 | TGGTG | | | | |
| CTC-1 | Nme2Cas9 | Nme37 | GCTACAGCAACAGGGT | 262 | 20 | GACCTC | GAPDH |
| | | -20 | GGTG | | | | |
| CTG-1 | Nme2Cas9 | Nme45 | GCCATATTGGAGATGAG | 263 | 26 | GCGCTG | SEC618 |
| | | -26 | GGTGGCAAG | | | | |
| CTG-1 | Nme2Cas9 | Nme45 | GCATATTGGAGATGAG | 264 | 25 | GCGCTG | SEC618 |
| | | -25 | GGTGGCAAG | | | | |
| CTG-1 | Nme2Cas9 | Nme45 | GATATTGGAGATGAGG | 265 | 24 | GCGCTG | SEC618 |
| | | -24 | GTGGCAAG | | | | |
| CTG-1 | Nme2Cas9 | Nme45 | GATTGGAGATGAGGGT | 266 | 22 | GCGCTG | SEC61B |
| | | -22 | GGCAAG | | | | |
| CTG-1 | Nme2Cas9 | Nme45 | GTTGGAGATGAGGGTG | 267 | 21 | GOGCTG | SEC61B |
| | | -21 | GCAAG | | | | |
| CTG-1 | Nme2Cas9 | Nme45 | GTGGAGATGAGGGTGG | 268 | 20 | GCGCTG | SEC618 |
| | | -20 | CAAG | | | | |
| ATT-1 | Nme2Cas9 | Nme48 | GGGGTGGGCTGTAACA | 269 | 26 | GAGATT | GRIN2B |
| | | -26 | GGAGGGCCAG | | | | |
| ATT-1 | Nme2Cas9 | Nme48 | GGGTGGGCTGTAACAG | 270 | 25 | GAGATT | GRIN28 |
| | | -25 | GAGGGCCAG | | | | |
| ATT-1 | Nme2Cas9 | Nme48 | GGTGGGCTGTAACAGG | 271 | 24 | GAGATT | GRIN28 |
| | | -24 | AGGGCCAG | | | | |
| ATT-1 | Nme2Cas9 | Nme48 | GGGGCTGTAACAGGAG | 272 | 22 | GAGATT | GRIN2B |
| | | -22 | GGCCAG | | | | |
| ATT-1 | Nme2Cas9 | Nme48 | GGGCTGTAACAGGAGG | 273 | 21 | GAGATT | GRIN2B |
| | | -21 | GCCAG | | | | |
| ATT-1 | Nme2Cas9 | Nme48 | GGCTGTAACAGGAGGG | 274 | 20 | GAGATT | GRIN2B |
| | | -20 | CCAG | | | | |
| ACC-1 | SpRY/SpRY- | SpRY1 | GCTGCATCCACCCCCCG | 275 | 20 | ACC | EMX1 |
| | HF1 | | AGG | | | | |
| CCC-1 | SpRY/SpRY- | SpRY2 | GCACAAGAGGAAGAGA | 276 | 20 | CCC | EMX1 |
| | HF1 | | GAGA | | | | |
| ACA-1 | SpRY/SpRY- | SpRY3 | GTACTTCGAAAAGGTAG | 277 | 20 | ACA | MECP2 |
| | HF1 | | GCG | | | | |
| GCA-1 | SpRY/SpRY- | SpRY4 | GAAGCAGGATGACAGG | 278 | 21 | GCA | Intergenic |
| | HF1 | | CAGGG | | | | |
| GCA-2 | SpRY/SpRY- | SpRY5 | GACCATGAGATAGAAG | 279 | 20 | GCA | SHANK3 |
| | HF1 | | GCGC | | | | |
| GCA-3 | SpRY/SpRY- | SpRY6 | GCTGGAGAAGCAGAAA | 280 | 20 | GCA | LINC01509 |
| | HF1 | | AAAA | | | | |
| TCA-1 | SpRY/SpRY- | SpRY7 | GCAGCAACAGGGTGGT | 281 | 21 | TCA | GAPDH |
| | HF1 | | GGACC | | | | |
| TCA-2 | SpRY/SpRY- | SpRY8 | GCAAGAGGAAGAGAGA | 282 | 21 | TCA | GAPDH |
| | HF1 | | GACCC | | | | |
| CCG-1 | SpRY/SpRY- | SpRY9 | GCTTTAACCCCCACCTCC | 283 | 20 | CCG | Intergenic |
| | HF1 | | AG | | | | |
| GCG-1 | SpRY/SpRY- | SpRY1 | GCAGACGGCAGTCACTA | 284 | 21 | GCG | VEGFA |
| | HF1 | 0 | GGGG | | | | |
| CCT-1 | SpRY/SpRY- | SpRY1 | GCTCCAATATGGTATGG | 285 | 21 | CCT | SEC61B |
| | HF1 | 1 | CGGC | | | | |
| GCT-1 | SpRY/SpRY- | SpRY1 | GCCAGTGGAGGCAAGA | 286 | 21 | GCT | FANCF |
| | HF1 | 2 | GGGCG | | | | |
| GCT-2 | SpRY/SpRY- | SpRY1 | GCACACACATCCCTCGT | 287 | 20 | GCT | MECP2 |
| | HF1 | 3 | GCA | | | | |
| TCT-1 | SpRY/SpRY- | SpRY1 | GAAGCAGGAGGAGTCA | 288 | 21 | TCT | SEC61B |
| | HF1 | 4 | GGGCT | | | | |
| CTA-1 | SpRY/SpRY- | SpRY1 | GAAGAAAGAGAGATGT | 289 | 20 | CTA | RUNX1 |
| | HF1 | 5 | AGGG | | | | |
| GTA-1 | SpRY/SpRY- | SpRY1 | GCAAATACCAGAGATAA | 290 | 21 | GTA | GRIN2B |
| | HF1 | 6 | GAGA | | | | |
| CTC-1 | SpRY/SpRY- | SpRY1 | GACAGCAACAGGGTGG | 291 | 21 | CTC | GAPDH |
| | HF1 | 7 | TGGAC | | | | |
| ATG-1 | SpRY/SpRY- | SpRY1 | GAGAGGGGAGAAGAAA | 292 | 21 | ATG | RUNX1 |
| | HF1 | 8 | GAGAG | | | | |
| CTG-1 | SpRY/SpRY- | SpRY1 | GAGATGAGGGTGGCAA | 293 | 20 | CTG | SEC61B |
| | HF1 | 9 | GGCG | | | | |
| GTG-1 | SpRY/SpRY- | SpRY2 | GTAGACAGGAGACAGC | 294 | 21 | GTG | FANCF |
| | HF1 | 0 | GCTGG | | | | |
| ATT-1 | SpRY/SpRY- | SpRY2 | GTGTAACAGGAGGGCC | 295 | 21 | ATT | GRIN2B |
| | HF1 | 1 | AGGAG | | | | |
| TTT-1 | SpRY/SpRY- | SpRY2 | GAGGCCCCAGAAGGAG | 296 | 21 | TTT | Intergenic |
| | HF1 | 2 | TCTGG | | | | |
| Site 1 | Nme2Cas9 | Nme50 | GCAGATCCCACAGGCGC | 32 | 23 | CAGTCG | PDCD1 |
| | | | CCTGGC | | | | |
| Site 1 | SpRY/SpRY- | SpRY2 | GATCCCACAGGCGCCCT | 58 | 20 | CAG | PDCD1 |
| | HF1 | 3 | GGC | | | | |
| Site 2 | Nme2Cas9 | Nme51 | GCTGCAGAAGGGATTCC | 75 | 23 | TGCGCG | FANCF |
| | | | ATGAGG | | | | |
| Site 2 | SpRY/SpRY- | SpRY2 | GCAGAAGGGATTCCAT | 76 | 20 | TGC | FANCF |
| | HF1 | 4 | GAGG | | | | |
| Site 3 | SpRY/SpRY- | SpRY2 | GCAAAGCAGGAGGAGT | 301 | 20 | GCT | SEC61B |
| | HF1 | 5 | CAGG | | | | |
| Site 4 | SpRY/SpRY- | SpRY2 | GAGGGCTGGAGAAGCA | 302 | 21 | AAA | LINC01509 |
| | HF1 | 6 | GAAAA | | | | |
| Site 5 | SpRY/SpRY- | SpRY2 | GTCCGCTTTAACCCCCA | 303 | 21 | CAG | Intergenic |
| | HF1 | 7 | CCTC | | | | |
| Site 6 | SpRY/SpRY- | SpRY2 | GTCATCTCCAATATGGT | 304 | 21 | CGG | SEC618 |
| | HF1 | 8 | ATGG | | | | |
| Nme2- | Nme2Cas9 | Nme51 | GCAATGGCCGGGACTCC | 305 | 23 | CACTCT | RBM20 |
| RBM1 | | | TGGGAG | | | | |
| Nme2- | Nme2Cas9 | Nme52 | GAATGGCCGGGACTCCT | 306 | 24 | CTCTCC | RBM20 |
| RBM2 | | | GGGAGCA | | | | |
| Nme2- | Nme2Cas9 | Nme53 | GATGGCCGGGACTCCTG | 307 | 24 | TCTCCC | RBM20 |
| RBM3 | | | GGAGCAC | | | | |
| SpRY- | SpRY/SpRY- | SpRY2 | GCCGGGACTCCTGGGA | 308 | 20 | TCT | RBM20 |
| RBM1 | HF1 | 9 | GCAC | | | | |
| SpRY- | SpRY/SpRY- | SpRY3 | GCCGGGACTCCTGGGA | 309 | 21 | CTC | RBM20 |
| RBM2 | HF1 | 0 | GCACT | | | | |
| SpRY- | SpRY/SpRY- | SpRY3 | GCGGGACTCCTGGGAG | 310 | 21 | TCC | RBM20 |
| RBM3 | HF1 | 1 | CACTC | | | | |
| SpRY- | SpRY/SpRY- | SpRY3 | GGGACTCCTGGGAGCA | 311 | 20 | CCC | RBM20 |
| RBM4 | HF1 | 2 | CTCT | | | | |

TABLE 3
Plasmids and selection phage (SP) used in Example 1

| | | | | ORF2 (prom |
|---|---|---|---|---|
| Name | Usage (resistance) | Origin | ORF1 (prom [RBS] genes) | [RBS] genes) |
| pTPH353e | AP validation (carbR) | SC101 | Pₚₛₚ⁵ [SD8⁶] gIII(1-18)-NpuN-32aa | |
| | | | linker-NpuC-gIII(18-425), luxAB | |
| pTPH353e-d | AP validation (carbR) | SC101 | Pₚₛₚ⁵ [SD8⁶] gIII(1-18)-NpuN-32aa | |
| | | | linker-NpuC(C1A)-gIII(18-425), luxAB | |
| pTPH353e-d stops | AP validation (carbR) | SC101 | Pₚₛₚ⁵ [SD8⁶] gIII(1-18)-NpuN-32aa linker | |
| | | | (double stop codon)-NpuC-gIII(18-425), | |
| | | | luxAB | |
| pTPH401 | AP validation (carbR) | ColE1 | P_{ProC}⁷ [sd8⁶] gIII(1-18)-NpuN-64aa | Plac Evo-1 |
| | | | linker(Evo-1 protospacer, NNNNCC | sgRNA |
| | | | PAM)-NpuC-gIII(18-425), luxAB | |
| pTPH400 | AP validation (carbR) | ColE1 | P_{ProC}⁷ [sd8⁶] gIII(1-18)-NpuN-121aa | Plac Evo-1 |
| | | | linker(Evo-1 protospacer, NNNNCC | sgRNA |
| | | | PAM)-NpuC-gIII(18-425), luxAB | |
| pTPH397 | AP validation (carbR) | ColE1 | P_{ProC}⁷ [sd8⁶] gIII(1-18)-NpuN-32aa | Plac Evo-1 |
| | | | linker(Evo-1 protospacer, NNNNCC | sgRNA |
| | | | PAM)-NpuC-gIII(18-425), luxAB | |
| pTPH397b | AP validation (carb^{R}) | ColE1 | P_{ProC}⁷ [sd8⁶] gIII(1-18)-NpuN-32aa | Plac Evo-2 |
| | | | linker(Evo-2 protospacer, NNNNCC | sgRNA |
| | | | PAM)-NpuC-gIII(18-425), luxAB | |
| pTPH397c | AP validation (carb^{R}) | ColE1 | P_{ProC}⁷ [sd8⁶] gIII(1-18)-NpuN-32aa | Plac Evo-3 |
| | | | inker(Evo-3 protospacer, NNNNCC | sgRNA |
| | | | PAM)-NpuC-gIII(18-425), luxAB | |
| pTPH405b | AP: ePACE1-2 (carb^{R}) | ColE1 | P_{ProC}⁷ [sd8⁶] gIII(1-18)-NpuN-32aa | Plac Evo-2 |
| | | | linker(Evo-2 protospacer, varied | sgRNA |
| | | | PAMs)-NpuC-gIII(18-425), luxAB | |
| pTPH405c | AP: ePACE3 (carb^{R}) | ColE1 | P_{ProC}⁷ [sd8⁶] gIII(1-18 recoded)-NpuN- | Plac Evo-2 |
| | | | 32aa linker(Evo-2 protospacer, varied | sgRNA |
| | | | PAMs)-NpuC-gIII(18-425), luxAB | |
| pTPH412 WT sd8 | CP validation (kan^{R}) | SC101 | Pₚₛₚ⁵ [sd8⁶] TadABE8e-gp41-8N | |
| pTPH412 WT sd2 | CP validation (kan^{R}) | SC101 | Pₚₛₚ⁵ [sd2⁶] TadABE8e-gp41-8N | |
| pTPH412 R26G sd8 | CP: ePACE3-5 (kan^{R}) | SC101 | Pₚₛₚ⁵ [sd8⁶] TadABE8e(R26G)-gp41-8N | |
| pTPH412 R26G sd2 | CP validation (kan^{R}) | SC101 | Pₚₛₚ⁵ [sd2⁶] TadABE8e(R26G)-gp41-8N | |
| pTPH418b | AP: ePACE4-5 (carb^{R}) | ColE1 | P_{ProC}⁷ [sd8⁶] gIII(1-18 recoded)-NpuN- | Plac Evo-4 |
| | | | 45aa linker(2 x Evo-4 protospacer, | sgRNA |
| | | | varied PAMs)-NpuC-gIII(18-425), luxAB | |
| pTPH343c | CBE-PPA (spec^{R}) | ColE1 | P_{BAD} [SD8⁶] rAPOBEC1-dSpCas9-UGI | Plac CBE-PPA |
| | | | | sgRNA |
| pTPH342 | CBE-PPA library (carb^{R}) | SC101 | CBE-PPA protospacer | PPro1 [SD8] |
| | | | | sfGFP |
| | | | | (inactive) |
| pTPH413 | ABE-PPA (cm^{R}) | p15A | P_{BAD} [sd4u⁶] TadABE8e-dNme2Cas9 | Plac ABE-PPA |
| | | | variant | sgRNA |
| pTPH424 | ABE-PPA library (carb^{R}) | SC101 | ABE-PPA protospacer | PPro1 [SD8] |
| | | | | sfGFP |
| | | | | (inactive) |
| Nme-IVT | IVT template (carb^{R}) | pUC | P_{T7(mutated)}⁸ Nme2ABE8e variant | |
| SpRY-IVT | IVT template (carb^{R}) | pUC | P_{T7(mutated)}⁸ SpRYABE8e or SpRY-HF1- | |
| | | | ABE8e variant | |
| Nme sgRNA | Mammalian guide expression | pUC | P_{hU6} Nme2Cas9 sgRNA | |
| | (carb^{R}) | | | |
| SpRY sgRNA | Mammalian guide expression | pUC | P_{hU6} SpRY/SpRY-HF1 sgRNA | |
| | (carb^{R}) | | | |
| Nme2 ABE8e variants | Mammalian expression of | pUC | P_{CMV} Nme2ABE8e variant | |
| | adenine base editor (carb^{R}) | | | |
| SpRYABE8e | Mammalian expression of | pUC | P_{CMV} SpRY/SpRY-HF1-ABE8e variant | |
| variants | adenine | | | |
| | base editor (carb^{R}) | | | |
| Nme2BE4 variants | Mammalian expression of | pUC | P_{CMV} Nme2BE4 variant | |
| | cytosine base editor (carb^{R}) | | | |
| SpRYBE4 variants | Mammalian expression of | pUC | P_{CMV} SpRY/SpRY-HF1-BE4 variant | |
| | adenine base editor (carb^{R}) | | | |
| Nme2 nuclease | Mammalian expression of | pUC | P_{CMV} Nme2Cas9 nuclease variant | |
| variants | nuclease (carb^{R}) | | | |
| SpRY nuclease | Mammalian expression of | pUC | P_{CMV} SpRY/SpRY-HF1 nuclease variant | |
| variants | nuclease | | | |
| | (carb^{R}) | | | |
| SP391c | ePACE1-2, □gIII SP, | M13 f1 | P_{gIII} [SD4⁴] TadABE8e-dNme2Cas9 | |
| | backbone recoded⁸ | | variant | |
| SP404 | ePACE3-5, □gIII SP, | M13 f1 | P_{gIII} [SD4⁴] gp41-C-dNme2Cas9 variant | |
| | backbone recoded⁷ | | | |

### REFERENCES

1. Wang, T., Badran, A. H., Huang, T. P. & Liu, D. R. Continuous directed evolution of proteins with improved soluble expression. Nat Chem Biol 14, 972-980, doi:10.1038/s41589-018-0121-5 (2018).
2. Miller, S. M., Wang, T. & Liu, D. R. Phage-assisted continuous and non-continuous evolution. Nature protocols 15, 4101-4127, doi:10.1038/s41596-020-00410-3 (2020).
3. Labun, K. et al. CHOPCHOP v3: expanding the CRISPR web toolbox beyond genome editing. Nucleic Acids Res 47, W171-W174, doi:10.1093/nar/gkz365 (2019).
4. Newby, G. A. et al. Base editing of haematopoietic stem cells rescues sickle cell disease in mice. Nature 595, 295-302, doi:10.1038/s41586-021-03609-w (2021).
5. Brissette, J. L., Weiner, L., Ripmaster, T. L. & Model. P. Characterization and sequence of the Escherichia coli stress-induced psp operon. J Mol Biol 220, 35-48, doi:10.1016/0022-2836(91)90379-k (1991).
6. Ringquist, S. et al. Translation initiation in Escherichia coli: sequences within the ribosome-binding site. Mol Microbiol 6, 1219-1229. doi:10.1111/j.1365-2958.1992.tb01561.x (1992).
7. Davis, J. H., Rubin, A. J. & Sauer, R. T. Design, construction and characterization of a set of insulated bacterial promoters. Nucleic Acids Res 39, 1131-1141, doi:10.1093/nar/gkg810 (2011).
8. Gaudelli, N. M. et al. Directed evolution of adenine base editors with increased activity and therapeutic application. Nature Biotechnology 38, 892-900, doi:10.1038/s41587-020-0491-6 (2020).
9. Richter, M. F., Zhao, K. T., Eton, E., Lapinaite, A., Newby, G. A., Thuronyi, B. W., Wilson, C., Zeng, J., Bauer, D. E., Doudna, J. A, Liu, D. R. Continuous evolution of an adenine base editor with enhanced Cas domain compatibility and activity. Nature Biotechnology, in press (2020).
10. Huang, T. P., Newby, 0. A. & Liu, D. R. Precision genome editing using cytosine and adenine base editors in mammalian cells. Nature Protocols 16, 1089-1128, doi:10.1038/s41596-020-00450-9 (2021).
11. Shen, W., Le, S., Li, Y. & Hu, F. SeqKit: A Cross-Platform and Ultrafast Toolkit for FASTA/Q File Manipulation. PLOS ONE 11, e0163962, doi:10.1371/journal.pone.0163962 (2016).
12. Clement, K. et al. CRISPResso2 provides accurate and rapid genome editing sequence analysis. Nature Biotechnology 37, 224-226, doi:10.1038/s41587-019-0032-3 (2019).

What is claimed is:

1. A Cas protein comprising an amino acid sequence that is at least 70%, at least 75%, at least 80%, at least 85%, at least 90%, at least 95%, at least 98% or at least 99% identical to the amino acid sequence of a Cas protein of SEQ ID NO: 5, wherein the amino acid sequence of the Cas protein comprises at least 1, at least 5, at least 10, at least 12, at least 15, at least 20, or at least 25 substitutions at amino acid positions selected from the group consisting of 6, 33, 47, 63, 68, 104, 116, 123, 152, 154, 221, 260, 263, 303, 396, 413, 427, 451, 452, 460, 484, 520, 629, 646, 674, 696, 711, 720, 724, 758, 765, 767, 769, 771, 816, 821, 844, 859, 865, 932, 940, 951, 1005, 1028, 1029, 1031, 1033, 1044, 1047, 1049, 1056, 1064, and 1075 of the amino acid sequence provided in SEQ ID NO: 5.

2. The Cas protein of claim 1, wherein the amino acid sequence of the Cas protein comprises at least 1, at least 5, at least 10, at least 12, at least 15, at least 20, or at least 25 substitutions selected from the group consisting of P6X, E33X, E47X, R63X, V68X, K104X, A116X, T123X, D152X, E154X, E221X, F260X, A263X, A303X, T396X, H413X, A427X, D451X, H452X, E460X, A484X, E520X, S629X, R646X, N674X, F696X, G711X, D720X, A724X, 1758X, V765X, H767X, K769X, H771X, S816X, V821X, D844X, 1859X, W865X, E932X, K940X, M951X, K1005X, D1028X, S1029X, N1031X, R1033X, K1044X, Q1047X, R1049X, V1056X, N1064X, and L1075X, relative to the amino acid sequence provided in SEQ ID NO: 5, wherein X represents any amino acid.

3. The Cas protein of claim 1 or 2, wherein the amino acid sequence of the Cas protein comprises at least 1, at least 5, at least 10, at least 12 at least 15, at least 20, or at least 25 substitutions selected from the group consisting of P6S, E33G, E47K, R63K, V68M, K104T, A116T, T123A, D152A, D152N, D112G, E154K, E221D, F260L, A263T, A303S, T396A, H413N, A427S, D451V, H452R, E460A, E460K, A484T, E520A, S629P, R646S, N674S, F696V, G711R, D720A, A724S, 1758V, V765A, H767Y, K769R, H771R, S8161, V821A, D844A, 1859V, W865L, E932K, K940R, M951R, K1005R, D1028N, S1029A, N1031S, R1033N, R1033G, R1033Y, K1044R, R1049S, R1049C, Q1047R, V1056A, N1064S, and L1075M, relative to the amino acid sequence of SEQ ID NO: 5.

4. The Cas protein of any one of claims 1-3, wherein the amino acid sequence of the Cas protein comprises substitutions at any of the following positions:
P6, E33, K104, D152, F260, A263, A303, D451, E520, R646, F696, G711, 1758, H767, E932, N1031, R1033, K1044, Q1047, and V1056, relative to the amino acid sequence of SEQ ID NO: 5.

5. The Cas protein of claim 4, wherein the amino acid sequence of the Cas protein comprises any of the following substitutions:
P6S, E33G, K104T, D152A, F260L, A263T, A303S, D451V, E520A, R646S, F696V, G711R, 1758V, H767Y, E932K, N1031S, R1033G, K1044R, Q1047R, and V1056A, relative to the amino acid sequence of SEQ ID NO: 5.

6. The Cas protein of any one of claims 1-5, wherein the amino acid sequence of the Cas protein comprises an amino acid sequence having at least 80%, at least 85%, at least 90%, at least 95%, at least 98%, or at least 99% identity to the amino acid sequence of SEQ ID NO: 1.

7. The Cas protein of any one of claims 1-6, wherein the amino acid sequence of the Cas protein comprises an amino acid sequence of SEQ ID NO: 1.

8. The Cas protein of any one of claims 1-3, wherein the amino acid sequence of the Gas protein comprises substitutions at any of the following positions:
K104, D152, F260, A263, A303, D451, E932, N1031, R1033, K1044, Q1047, and V1056, relative to the amino acid sequence of SEQ ID NO: 5.

9. The Cas protein of claim 8, wherein the amino acid sequence of the Cas protein comprises any of the following substitutions:
K104T, D152A, F260L, A263T, A303S, D451V, E932K, N1031S, R1033G, K1044R, Q1047R, and V1056A, relative to the amino acid sequence of SEQ ID NO: 5.

10. The Cas protein of any one of claims 1-9, wherein the amino acid sequence of the Cas protein comprises an amino acid sequence having at least 80%, at least 85%, at least 90%, at least 95%, at least 98%, or at least 99% identity to the amino acid sequence of SEQ ID NO: 4.

11. The Cas protein of any one of claims 1-10, wherein the amino acid sequence of the Cas protein comprises an amino acid sequence of SEQ ID NO: 4.

12. The Cas protein of any one of claims 1-3, wherein the amino acid sequence of the Gas protein comprises substitutions at any of the following positions:
E47, V68, T123, D152, E154, T396, H413, A427, H452, E460, A484, 5629, N674, D720, V765, H767, H771, V821, D844, 1859, W865, M951, K1005, D1028, S1029, R1033, R1049, and N1064, relative to the amino acid sequence of SEQ ID NO: 5.

13. The Cas protein of claim 12, wherein the amino acid sequence of the Cas protein comprises any of the following substitutions:
E47K, V68M, T123A, D152G, E154K, T396A, H413N, A427S, H452R, E460A, A484T, S629P, N674S, D720A, V765A, H767Y, H771R, V821A, D844A, 1859V, W865L, M951R, K1005R, D1028N, S1029A, R1033Y, R1049S, and N1064S, relative to the amino acid sequence of SEQ ID NO: 5.

14. The Cas protein of any one of claims 1-13, wherein the amino acid sequence of the Cas protein comprises an amino acid sequence having at least 80%, at least 85%, at least 90%, at least 95%, at least 98% or at least 99% identity to the amino acid sequence of SEQ ID NO: 2.

15. The Cas protein of any one of claims 1-14, wherein the amino acid sequence of the Cas protein comprises an amino acid sequence of SEQ ID NO: 2.

16. The Cas protein of any one of claims 1-3, wherein the amino acid sequence of the Cas protein comprises substitutions at any of the following positions:
E47, R63, V68, A116, T123, D152, E154, E221, T396, H452, E460, N674, D720, A724, K769, S816, D844, E932, K940, M951, K1005, D1028, S1029, R1033, R1049, and L1075, relative to the amino acid sequence of SEQ ID NO: 5.

17. The Cas protein of claim 16, wherein the amino acid sequence of the Cas protein comprises any of the following substitutions:
E47K, R63K, V68M, A116T, T123A, D152N, E154K, E221D, T396A, H452R, E460K, N674S, D720A, A724S, K769R, S8161, D844A, E932K, K940R, M951R, K1005R, D1028N, S1029A, R1033N, R1049C, and L1075M, relative to the amino acid sequence of SEQ ID NO: 5.

18. The Cas protein of any one of claims 1-17, wherein the amino acid sequence of the Cas protein comprises an amino acid sequence having at least 80%, at least 85%, at least 90%, at least 95%, at least 98% or at least 99% identity to the amino acid sequence of SEQ ID NO: 3.

19. The Cas protein of any one of claims 1-18, wherein the amino acid sequence of the Cas protein comprises an amino acid sequence of SEQ ID NO: 3.

20. The Cas protein of any one of claims 1-19, wherein the Cas protein exhibits increased activity on a target sequence as compared to a wild-type Nme2Cas9 protein as provided by SEQ ID NO: 5.

21. The Cas protein of any one of claims 1-20, wherein the Cas protein exhibits an activity on a target sequence that is increased by at least 2-fold, at least 3-fold, at least 4-fold, at least 5-fold, at least 6-fold, at least 7-fold, at least 8-fold, at least 9-fold, or at least 10-fold as compared to a wild-type Nme2Cas9 protein as provided by SEQ ID NO: 5.

22. A fusion protein comprising:
the Cas protein of any one of claims 1-21; and
an effector domain.

23. The fusion protein of claim 22, wherein the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

24. The fusion protein of claim 22 or 23, wherein the effector domain is a nucleic acid editing domain.

25. The fusion protein of claim 24, wherein the nucleic acid editing domain comprises a deaminase domain.

26. The fusion protein of claim 25, wherein the deaminase domain is an adenosine deaminase domain.

27. The fusion protein of claim 26, wherein the adenosine deaminase domain is an E. coli TadA (ecTadA) deaminase domain.

28. The fusion protein of any one of claims 22-27, wherein the fusion protein is an adenine base editor (ABE).

29. The fusion protein of any one of claims 22-28, wherein the base editor comprises the structure: NH₂-[adenosine deaminase]-[Cas9 protein]-COOH; or NH₂-[Cas9 protein]-[adenosine deaminase]-COOH, wherein each “]-[” in the structure indicates the presence of an optional linker sequence.

30. The fusion protein of claim 25, wherein the deaminase domain is a cytidine deaminase domain.

31. The fusion protein of claim 30, wherein the cytidine deaminase domain is an apolipoprotein B mRNA-editing complex (APOBEC) family deaminase domain.

32. The fusion protein of any one of claims 22-25, 30, and 31, wherein the fusion protein is a cytosine base editor (CBE).

33. The fusion protein of any one of claims 22-28, wherein the base editor comprises the structure: NH₂-[cytidine deaminase]-[Cas9 protein]-COOH; or NH₂-[Cas9 protein]-[cytidine deaminase]-COOH, wherein each “]-[” in the structure indicates the presence of an optional linker sequence.

34. The fusion protein of claim 29 or 33 further comprising one or more linkers between the Cas9 protein and the adenosine deaminase.

35. The fusion protein of claim 34, wherein the one or more linkers comprises an amino acid sequence selected from SGGSSGGSSGSETPGTSESATPESSGGSSGGS (SEQ ID NO: 412), GGG, SGGS (SEQ ID NO: 413), GGGS (SEQ ID NO: 430), SGGGS (SEQ ID NO: 431), and SGSETPGTSESATPES (SEQ ID NO: 422).

36. The fusion protein of any one of claims 22-35, wherein the linker is SEQ ID NO: 412.

37. The fusion protein of any one of claims 22-35 further comprising one or more nuclear localization sequences (NLS).

38. The fusion protein of claim 37, wherein the one or more nuclear localization sequences comprises an amino acid sequence selected from PKKKRKV (SEQ ID NO: 142), MDSLLMNRRKFLYQFKNVRWAKGRRETYLC (SEQ ID NO: 144), KRTADGSEFESPKKKRKV (SEQ ID NO: 153), and KRTADGSEFEPKKKRKV (SEQ ID NO: 155).

39. The fusion protein of any one of claims 22-38, wherein the base editor comprises the structure: NH2-[first NLS]-[cytidine deaminase]-[Cas9 protein]-[second NLS]-COOH; or NH2-[first NLS]-[Cas9 protein]-[cytidine deaminase]-[second NLS]-COOH.

40. The fusion protein of any one of claims 22-39, wherein the fusion protein comprises a first NLS and a second NLS.

41. The fusion protein of claim 39 or 40, wherein the first NLS is SEQ ID NO: 142.

42. The fusion protein of claim 39 or 40, wherein the second NLS is SEQ ID NO: 155.

43. A guide RNA (gRNA) comprising, a nucleic acid sequence comprising a spacer sequence and a scaffold sequence, wherein the spacer sequence comprises a nucleic acid sequence that is at least 80%, at least 85%, at least 90%, at least 95%, at least 98% or at least 99% identical to the nucleic acid sequence of any one of the spacers in Table 2.

44. The gRNA of claim 43, wherein the spacer sequence comprises a nucleic acid sequence that differs by 1, 2, 3, 4, 5, 6, 7, 8, 9, or 10 nucleotides relative to of any one of the spacers in Table 2.

45. The gRNA of claim 43 or 44, wherein the spacer sequence comprises a nucleic acid sequence that comprises the nucleic acid sequence of any one of the spacers in Table 2.

46. The gRNA of claim 43-45, wherein the scaffold sequence comprises a nucleic acid sequence that comprises the nucleic acid sequence of SEQ ID NO: 100.

47. The gRNA of claim 43, wherein the nucleic acid sequence comprising a spacer sequence and a scaffold sequence is at least 70%, at least 75%, at least 80%, at least 85%, at least 90%, at least 95%, at least 98% or at least 99% identical to the nucleic acid sequence of any one of SEQ ID NOs: 200-205.

48. The gRNA of claim 43-47, wherein the nucleic acid sequence comprising a spacer sequence and a scaffold sequence comprises the nucleic acid sequence of any one of SEQ ID NOs: 200-205.

49. A complex comprising a fusion protein of any one of claims 22-42 and a guide RNA.

50. A complex comprising a fusion protein and the guide RNA of any one of claims A38-A44.

51. A complex comprising a fusion protein of any one of claims 22-42 and a guide RNA of any one of claims 43-48.

52. A method for editing a target nucleic acid molecule comprising contacting the target nucleic acid molecule with the fusion protein of any one of claims 22-42 and the guide RNA of any one of claims 43-48.

53. The method of claim 52, wherein the guide RNA comprises a nucleic acid sequence of any one of SEQ ID NOs: 200-205, or a nucleic acid sequence that is at least at least 70%, at least 75%, at least 80%, at least 85%, at least 90%, at least 95%, at least 98% or at least 99% identical to the nucleic acid sequence of any one of SEQ ID NOs: 200-205.

54. A method for editing a target nucleic acid molecule comprising contacting the target nucleic acid molecule with the complex of any one of claims 49-51.

55. The method of any one of claims 52-54, wherein the contacting is performed in vitro.

56. The method of any one of claims 52-54, wherein the contacting is performed in vivo.

57. The method of claim 56, wherein the contacting is performed in a subject.

58. The method of claim 57, wherein the subject has been diagnosed with a disease or disorder.

59. The method of any one of claims 52-58, wherein the target sequence comprises a genomic sequence associated with a disease or disorder.

60. The method of claim 59, wherein the target sequence comprises a point mutation associated with a disease or disorder.

61. The method of claim 60, wherein the point mutation comprises a T to C point mutation associated with a disease or disorder.

62. The method of any one of claims 60 or 61, wherein the point mutation comprises a C to T point mutation associated with a disease or disorder.

63. The method of claim 60, wherein the point mutation comprises an A to G point mutation associated with a disease or disorder.

64. The method of any one of claims 60 or 63, wherein the point mutation comprises an G to A point mutation associated with a disease or disorder.

65. The method of any one of claims 52-64, wherein the step of editing the target nucleic acid results in correction of the point mutation.

66. A polynucleotide encoding the Cas protein of any one of claims 1-21, the fusion protein of any one of claims 22-42, the guide RNA of any one of claims 43-48, or a complex of any one of claims 49-51.

67. A polynucleotide comprising a (i) first segment encoding the fusion protein of any one of claims 22-42 and (ii) a second segment encoding the guide RNA of any one of claims 43-48.

68. A vector comprising the polynucleotide of claim 66 or 67.

69. The vector of claim 68, wherein the vector is an adeno-associated viral (AAV) vector.

70. An AAV vector comprising the polynucleotide of claim 67, wherein the orientation of the second segment is reversed relative to the first segment.

71. A recombinant adeno-associated viral (rAAV) particle comprising the AAV vector of claim 69 or 70.

72. A cell comprising a Cas protein of any one of claims A1-A21, a fusion protein of any one of claims 22-42, a guide RNA of any one of claims 43-48, a complex of any one of claims 49-51, a polynucleotide of claim 66 or 67, a vector of any one of claims 68-70, or an rAAV particle of claim 71.

73. A kit comprising a nucleic acid construct, comprising
(a) a nucleic acid sequence encoding the fusion protein of any one of claims 22-42;
(b) a nucleic acid sequence encoding a gRNA; and
(c) one or more heterologous promoters that drive the expression of the sequence of (a) and/or the sequence of (b).

74. A pharmaceutical composition comprising a Cas protein of any one of claims 1-21, a fusion protein of any one of claims 22-42, a guide RNA of any one of claims 43-48, a complex of any one of claims 49-51, a polynucleotide of claim 66 or 67, a vector of any one of claims 68-70, an rAAV particle of claim 71, or a cell of claim 72, and a pharmaceutically acceptable excipient.

75. A Cas protein of any one of claims 1-21, a fusion protein of any one of claims 22-42, a guide RNA of any one of claims 43-48, a complex of any one of claims 49-51, a polynucleotide of claim 66 or 67, a vector of any one of claims 68-70, an rAAV particle of claim 71, a cell of claim 72, or a pharmaceutical composition of claim 74 for use in medicine.

76. Use of a Cas protein of any one of claims 1-21, a fusion protein of any one of claims 22-42, a guide RNA of any one of claims 43-48, a complex of any one of claims 49-51, a polynucleotide of claim or 67, a vector of any one of claims 68-70, an rAAV particle of claim 71, a cell of claim 72, or a pharmaceutical composition of claim 72 in the manufacture of a medicament for the treatment of a disease or disorder.

77. The use of as Cas protein of claim 76, wherein the disease or disorder is sickle cell disease (SCD).

78. The use of as Cas protein of claim 77, wherein the sickle cell disease (SCD) is caused by a mutation in a gene locus.

79. The use of as Cas protein of claim 78, wherein the gene locus is a mutation of the mammalian β-globin (HBB) gene locus at amino acid position 6, relative to the wild-type mammalian β-globin (HBB) gene.

80. The use of as Cas protein of claim 79, wherein the mutation of the mammalian βglobin (HBB) gene locus at amino acid position 6, is a glutamate to valine mutation.

81. A vector system for phage-assisted continuous evolution comprising:
a. a vector containing a nucleic acid that encodes a fusion protein;
b. a vector containing a nucleic acid that encodes a bacteriophage (phage) gene essential for phage propagation and a nucleic acid sequence encoding an in cis split intein positioned within the coding sequence of the gene; and
c. a mutagenesis plasmid.

82. The vector system of claim 81, wherein the nucleic acid sequence encoding the in cis split intein is inserted between amino acid positions 10 and 11 of the coding sequence of the phage gene.

83. The vector system of any one of claims 81-82, wherein the nucleic acid sequence encoding the in cis split intein is inserted between amino acid positions 18 and 19 of the coding sequence of the phage gene.

84. The vector system of any one of claims 81-83, wherein the gene essential for phage propagation is gene III (gIII).

85. The vector system of any one of claims 81-84, wherein the nucleic acid sequence encoding an in cis split intein comprises a nucleic acid sequence encoding an intein N-terminal (Int-N), connected by a polynucleotide insert sequence to a nucleic acid sequence encoding an intein C-terminal (Int-C).

86. The vector system of claim 85, wherein the polynucleotide insert sequence comprises an amino acid sequence that is between 32-121 amino acids in length.

87. The vector system of claim 85 or 86, wherein the polynucleotide insert sequence comprises and amino acid sequence that is 32 amino acids in length.

88. The vector system of any one of claims 85-87, wherein the polynucleotide insert sequence comprises a nucleotide sequence comprising at least 1, at least 2, at least 3 or at least 4 stop codons.

89. The vector system of any one of claims 86-88, wherein the nucleic acid sequence encoding Int-N and Int-C are from N. punctiforme (Npu).

90. The vector system of any one of claims 81-89, wherein the vector containing a nucleic acid that encodes a gene essential for phage propagation and an intein comprise at least 1 protospacer and at least 1 PAM sequence.

91. The vector system of any one of claims 81-90, wherein the polynucleotide insert sequence comprises at least 1 protospacer and at least 1 PAM sequence.

92. The vector system of any one of claims 81-91, wherein the protospacer comprises a nucleotide sequence comprising at least 1 disease-relevant site.

93. The vector system of any one of claims 81-92, wherein the protospacer comprises a nucleotide sequence comprising at least 1, at least 2, at least 3 or at least 4 stop codons.

94. The vector system of any one of claims 81-93, wherein the disease-relevant site is a mammalian CFTR locus.

95. The vector system of any one of claims 81-94, wherein the at least 1, at least 2, at least 3 or at least 4 stop codons comprise an R1162X mutation in the mammalian CFTR locus, wherein X is any amino acid other than R.

96. The vector system of any one of claims 81-95, wherein the fusion protein comprises a Cas9 protein.

97. The vector system of any one of claims 81-96, wherein the fusion protein comprises a Nme2Cas9 protein.

98. The vector system of any one of claims 81-97, wherein the vector system further comprises
(d) a vector containing a nucleic acid that encodes a second bacteriophage (phage) gene that prevents phage propagation and a nucleic acid sequence encoding a second in cis split intein positioned within the coding sequence of the gene.

99. The vector system of claim 98, wherein the nucleic acid sequence encoding the second in cis split intein is inserted between amino acid positions 18 and 19 of the coding sequence of the second phage gene.

100. The vector system of any one of claims 97-99, wherein the second bacteriophage (phage) gene that prevents phage propagation is gene III (gIII)-neg.

101. The vector system of any one of claims 97-100, wherein the nucleic acid sequence encoding the second in cis split intein comprises a second nucleic acid sequence encoding an intein N-terminal (Int-N), connected by a second polynucleotide insert sequence to a nucleic acid sequence encoding an intein C-terminal (Int-C).

102. The vector system of claim 101, wherein the polynucleotide insert sequence comprises an amino acid sequence that is between 32-121 amino acids in length.

103. The vector system of claim 101 or 102, wherein the polynucleotide insert sequence comprises a nucleotide sequence comprising at least 1, at least 2, at least 3, or at least 4 stop codons.

104. The vector system of any one of claims 100-103, wherein the vector comprises a nucleic acid that encodes a second bacteriophage (phage) gene that prevents phage propagation and a nucleic acid sequence encoding a second in cis split intein positioned within the coding sequence of the gene comprise at least 1 protospacer and at least 1 PAM sequence.

105. A cell comprising the vector system of any one of claims 81-104.

106. A method of continuous evolution comprising:
a. introducing a selection phage encoding a nucleic acid that encodes a fusion protein into a flow of a population of host cells through a lagoon,
wherein the population of host cells comprise a phage gene essential for phage propagation, wherein the phage gene comprises a coding sequence comprising at least 1 stop codon and an in cis split intein,
wherein the phage gene essential for phage propagation is expressed in response to contacting the population of host cells with the selection phage encoding a nucleic acid that encodes the fusion protein and the at least 1 stop codon is corrected, and
wherein the flow rate of the population of host cells through the lagoon permits replication of the phage with the at least 1 stop codon corrected, but not of the host cells, in the lagoon;
b. replicating and mutating the selection phage within the flow of host cells; and
c. isolating a selection phage comprising a mutated gene to be evolved from the flow of cells.

107. The method of continuous evolution of claim 106, wherein steps a.-c. are performed in an automated continuous culture platform.

108. The method of continuous evolution of claim 107, wherein the automated continuous culture platform comprises any of an eVOLVER unit, an Integrated Peristaltic Pump (IPP) device, media/efflux pumps, an inducer, a pressure regulator, and a solenoid bank.

109. The method of continuous evolution of claim 107, wherein the automated continuous culture platform comprises an eVOLVER unit, an Integrated Peristaltic Pump (IPP) device, media/efflux pumps, an inducer, a pressure regulator, and a solenoid bank.

110. The method of continuous evolution of any one of claims 107-109, wherein the automated continuous culture platform is comprised of a fluidic layer and a control layer.

111. The method of continuous evolution of any one of claims 107-110, wherein the automated continuous culture platform is bonded using an adhesive material.

112. The method of continuous evolution of any one of claims 110-111, wherein the fluidic layer and the control layer are fabricated using a laser-cutting method.

113. The method of continuous evolution of any one of claims 110-112, wherein the fluidic layer and the control layer are fabricated using an acrylic material.

114. The method of continuous evolution of claim 107, wherein the automated continuous culture platform comprises a series of integrated peristaltic pumps (IPPs) that control a flow rate.

115. The method of continuous evolution of claim 114, wherein the flow rate is in the range of less than 0.1 to 40 μL/s.

116. The method of continuous evolution of any one of claims 107-115, wherein the IPP device is manufactured using laser cutting.

117. The method of continuous evolution of any one of claims 107-116, wherein the IPP device comprises a sequential actuation of consecutively-arranged pneumatic valves.

118. The method of continuous evolution of any one of claims 107-117, wherein the sequential actuation of consecutively-arranged pneumatic valves occurs in a “100, 010, 001” pattern, where “0” indicates “valve open,” and “1” indicates “valve closed”.

119. The method of continuous evolution of any one of claims 107-116, wherein the automated continuous culture platform comprises a pressure regulator.

120. The method of continuous evolution of any one of claims 107-119, wherein the pressure regulator comprises a modular architecture via a millifluidic interface with the valves.

121. The method of continuous evolution of any one of claims 107-120, wherein the pressure regulator has up to 16 proportional valves that can be used for pressure regulation up to 8 channels.

122. The method of continuous evolution of any one of claims 107-121, wherein the pressure regulator comprises multiple pressure regulators that can be chained together to regulate an arbitrary number of pressure channels.

123. The method of continuous evolution of claim 119, wherein the pressure regulator comprises:
(a) a set of two proportional valves that can limit air flow from a high-pressure source and a vent at atmospheric pressure;
(b) an electronic pressure gauge on the output of the set of two proportional valves;
wherein, proportional-integral-derivative (PID) control over the valves set the output pressure to any desired level between the input and atmospheric pressure.

124. The method of any one of claims 106-123, wherein, the phage gene is a gene encoding gill protein.

125. The method of any one of claims 106-124, wherein the in cis split intein comprises a polynucleotide insert sequence, at least 1 protospacer sequence, and at least 1 PAM sequence.

126. The method of any one of claims 106-125, wherein the in cis split intein is inserted between amino acid positions 10 and 11 of the coding sequence of gill protein.

127. The method of any one of claims 106-126, wherein the in cis split intein is inserted between amino acid positions 18 and 19 of the coding sequence of gII protein.

128. The method of any one of claims 106-127, wherein the in cis split intein comprises Int-N and Int-C of an intein from N. punciforme (Npu).

129. The method of any one of claims 125-128, wherein the polynucleotide insert sequence is between 32-121 amino acids in length.

130. The method of any one of claims 125-129, wherein the polynucleotide insert sequence is 32 amino acids in length.

131. The method of any one of claims 125-130, wherein the polynucleotide insert sequence comprises at least 1 or at least 2 stop codons.

132. The method of any one of claims 106-131, wherein the phage gene comprises a coding sequence with altered codon usage in a N-terminal region.

133. The method of claim 132, wherein the N-terminal region is between amino acid positions 1-18.

134. The method of any one of claims 132 or 133, wherein the N-terminal region comprises a sub-region of altered nucleotide homology.

135. The method of any one of claims 132-134, wherein the in cis split intein comprises 2 protospacers, each flanked by a PAM sequence and comprising alternate sequence identity at PAM nucleic acid positions 1-3 and 7.

136. The method of any one of claims 132-135, wherein the selection phage comprises a fusion protein comprising a TadA8e domain and a dNme2Cas9 domain connected by a polynucleotide insert and an in trans intein.

137. The method of claim 136, wherein the in trans intein is gp41-8.

138. A vector comprising a nucleic acid coding sequence that encodes a gIII protein comprising an in cis split intein pair connected by a polynucleotide insert sequence, at least 1 protospacer sequence, and at least 1 PAM sequence.

139. The vector of claim 138, wherein the in cis intein pair is inserted between nucleotide positions 30 and 31 of the coding sequence of gIII protein.

140. The vector of claim 138 or 139, wherein the in cis intein pair is inserted between nucleotide positions 54 and 55 of the coding sequence of gIII protein.

141. The vector of any one of claims 138-140, wherein the in cis intein comprises Int-N and Int-C of an intein from N. punciforme (Npu).

142. The vector of any one of claims 138-141, wherein the polynucleotide insert sequence is between 32-121 amino acids in length.

143. The vector of any one of claims 138-142, wherein the polynucleotide insert sequence is 32 amino acids in length.

144. The vector of any one of claims 138-143, wherein the polynucleotide insert sequence comprises at least 1 or at least 2 stop codons.

145. The vector of claim 138, wherein the nucleic acid sequence has at least 80%, at least 85%, at least 90%, at least 95%, at least 98%, or at least 99% identity to the nucleic acid sequence of SEQ ID NO: 8.

146. The vector of claim 138, wherein the nucleic acid sequence comprises the nucleic acid sequence of SEQ ID NO: 8.

147. The vector of any one of claims 138-146, wherein an N-terminal region of the coding sequence comprises altered codon usage.

148. The vector of any one of claims 138-147, wherein the N-terminal region comprises a sub-region of altered nucleotide homology.

149. The vector of claim 138, wherein the nucleic acid sequence has at least 80%, at least 85%, at least 90%, at least 95%, at least 98%, or at least 99% identity to the nucleic acid sequence of SEQ ID NO: 9.

150. The vector of claim 138, wherein the nucleic acid sequence comprises the nucleic acid sequence of SEQ ID NO: 9.

151. The vector of any one of claims 138-150, wherein there are 2 protospacers, each flanked by a PAM sequence and comprising alternate sequence identity at PAM nucleic acid positions 1-3 and 7.

152. The vector of claim 138, wherein the nucleic acid sequence has at least 80%, at least 85%, at least 90%, at least 95%, at least 98%, or at least 99% identity to the nucleic acid sequence of SEQ ID NO: 7.

153. The vector of claim 138, wherein the nucleic acid sequence comprises the nucleic acid sequence of SEQ ID NO: 7.

154. One or more vectors comprising a nucleic acid sequence that encodes a fusion protein comprising a TadA8e domain and a dNme2Cas9 domain connected by a polynucleotide insert sequence and an in trans intein.

155. The one or more vectors of claim 154, wherein the in trans intein is gp41-8.

156. The one or more vectors of claim 138, wherein the nucleic acid sequence has at least 80%, at least 85%, at least 90%, at least 95%, at least 98%, or at least 99% identity to the nucleic acid sequence of SEQ ID NO: 10.

157. The one or more vectors of any one of claim 138, wherein the nucleic acid sequence comprises the nucleic acid sequence of SEQ ID NO: 10.

158. The vector of any one of claims 138-157, wherein the vector is used as an accessory in a phage-assisted continuous evolution (PACE) selection method of continuous evolution.

159. The vector of any one of claims 138-158, or the vector system of any one of claims 81-97, wherein the vector comprises a promoter sequence.

160. The vector of any one of claims 138-159, or the vector system of any one of claims 81-97, wherein the promoter sequence comprises one of the following: a phage shock promoter (psp) sequence, a proD sequence, a proC sequence, or a pro5 sequence, optionally the psp sequence.

161. A method comprising transforming cells with a base editing (BE)-expressing plasmid (BP) and a library plasmid (LP), and further subjecting these cells to (a) an induction, (b) signal amplification, (c) harvesting, and (d) sequence analysis.

162. The method of claim 161, wherein the BE-expressing plasmid (BP) comprises an sgRNA, a promoter, and a base editor construct.

163. The method of claim 162, wherein the base editor construct encodes an adenine base editor.

164. The method of claim 162, wherein the base editor construct encodes a cytosine base editor.

165. The method of claim 162, wherein the promoter is a pBAD.

166. The method of claim 161, wherein the library plasmid (LP) comprises a protospacer, a target base and a PAM library.

167. The method of claim 161, wherein the sequence analysis comprises a CRISPResso2 analysis.
